(19) United States
(12) Patent Application Publication
(10) Pub. No.: US 2025/0129034 A1
(43) Pub. Date: Apr. 24, 2025

(54) ORGANIC COMPOUND AND ORGANIC LIGHT EMITTING DIODE COMPRISING SAME

(71) Applicant: MATERIAL SCIENCE CO.,LTD., Seoul (KR)

(72) Inventors: Tae Wan LEE, Seoul (KR); Jie SONG, Seoul (KR); Hani JEON, Seoul (KR); Tae-ho KWAK, Seoul (KR)

(21) Appl. No.: 18/906,085

(22) Filed: Oct. 3, 2024

Related U.S. Application Data
(63) Continuation of application No. PCT/KR2023/004631, filed on Apr. 5, 2023.

(30) Foreign Application Priority Data
Apr. 5, 2022 (KR) 10-2022-0042440
Apr. 5, 2023 (KR) 10-2023-0044903

(51) Int. Cl. C07D 307/91; C07B 59/00; C07D 405/12; C07D 407/12; C07D 409/12; C07F 7/08; H10K 50/15; H10K 85/40; H10K 85/60

(52) U.S. Cl. CPC C07D 307/91; C07B 59/004; C07D 405/12; C07D 407/12; C07D 409/12; C07F 7/0812; H10K 50/15; H10K 85/40; H10K 85/615; H10K 85/633; H10K 85/6572; H10K 85/6574; H10K 85/6576; C07B 2200/05

(57) ABSTRACT

An organic compound represented by chemical formula 1 of the present invention can implement excellent hole transfer characteristics. In addition, at least one of a hole transport layer and a hole transport auxiliary layer of an organic light emitting diode of the present invention comprises the organic compound represented by chemical formula 1 of the present invention, and thus can improve operation voltage, efficiency, and lifetime characteristics of the organic light emitting diode and, even when combined with a light emitting layer of any color, can implement excellent color coordinates targeted by the light emitting layer.

### CROSS-REFERENCE TO RELATED APPLICATION

The present application is a Continuation of PCT application number PCT/KR2023/004631, filed on Apr. 5, 2023, which is based upon and claims the benefit of priorities to Korean Patent Application Nos. 10-2022-0042440, filed on Apr. 5, 2022, and 10-2023-0044903, filed on Apr. 5, 2023, in the Korean Intellectual Property Office. All of the aforementioned applications are hereby incorporated by reference in their entireties.

### TECHNICAL FIELD

present invention relates to an organic compound, and more specifically, to an organic compound with hole transport characteristics, and an organic light emitting diode including the same.

### BACKGROUND

Interest in display devices is increasing according to the application to various fields. As one of the display devices, a technology of an organic light emitting display devices including an organic light emitting diode (OLED) is developing rapidly.

The OLED is a device for, when charges are injected into a light emitting layer formed between an anode and a cathode, emitting energies of excitons as light after forming electrons and holes in pair to form excitons. Compared to conventional display technologies, the OLED can implement a low voltage, consume relatively less power, have excellent colors, can be applied on a flexible substrate to be used variously, and can allow a display device to be freely adjusted in size.

The OLED can have an excellent viewing angle, contrast ratio, and the like compared to liquid crystal displays (LCDs) and do not require a backlight, making it lightweight and ultra-thin. The OLED is formed by arranging a plurality of organic layers, such as a hole injection layer, a hole transport layer, a hole transport auxiliary layer, an electron blocking layer, a light emitting layer, and an electron transport layer, between the cathode (electrode injection layer) and the anode (hole injection layer).

In the structure of the OLED, when a voltage is applied between two electrodes, electrons and holes are injected from the cathode and the anode, respectively, and excitons generated from the light emitting layer fall to a ground state to emit light.

Organic materials used in the OLED may be largely classified into light emitting materials and charge transport materials. The light emitting material is an important factor in determining the light emitting efficiency of the OLED, and the light emitting material should have high quantum efficiency, excellent mobility of electrons and holes, and exist uniformly and stably in the light emitting layer. The light emitting material is classified into light emitting materials such as blue, red, and green depending on colored light, and hosts and dopants are used to increase color purity and increase luminous efficiency through energy transfer as color materials.

In the case of fluorescent materials, only singlets of about 25% of the excitons formed in the light emitting layer are used to generate light, and triplets of 75% are mostly lost as heat, while phosphorescent materials has a luminous mechanism which converts both the singlets and the triplets into light.

The present invention is directed to providing an organic compound with hole transport characteristics.

In addition, the present invention is directed to providing an organic light emitting diode capable of improving operation voltage, efficiency, and lifetime characteristics by applying the organic compound to any one or more of a hole transport layer and a hole transport auxiliary layer.

In addition, the present invention is directed to providing an organic light emitting diode capable of implementing excellent color coordinates targeted by an any light emitting layer even when the hole transport layer and/or hole transport auxiliary layer including the organic compound are combined with a light emitting layer of any color.

Objects of the present invention are not limited to the above-described objects, and other objects and advantages of the present invention which are not mentioned can be understood by the following description and more clearly understood by embodiments of the present invention. In addition, it can be easily seen that the objects and advantages of the present invention can be achieved by means and combinations thereof which are described in the claims.

### SUMMARY

To achieve the objects, according to one aspect of the present invention, there may be provided an organic compound with a novel structure represented by Chemical Formula 1 below, and a definition of the Chemical Formula 1 below is the same as one described in the specification and the claims.

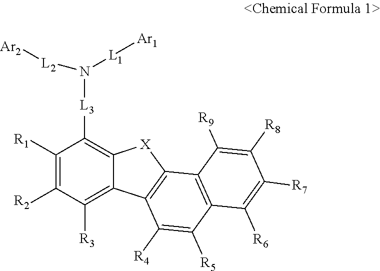

According to another aspect of the present invention, there may be provided an organic light emitting diode including a first electrode, a second electrode facing the first electrode, and an organic layer disposed between the first electrode and the second electrode, wherein the organic layer may include one or more of a hole transport layer and a hole transport auxiliary layer, and the one or more of the hole transport layer and the hole transport auxiliary layer include an organic compound represented by Chemical Formula 1.

### Advantageous Effects

The organic compound represented by chemical formula 1 of the present invention can implement excellent hole transfer characteristics.

In addition, the one or more of the hole transport layer and the hole transport auxiliary layer of the organic light emitting diode of the present invention can improve operation voltage, efficiency, and lifetime characteristics of the organic light emitting diode by including the organic compound represented by Chemical Formula 1 of the present invention.

In addition, the organic light emitting diode of the present invention can implement excellent color coordinates targeted by the light emitting layer even when the hole transport layer and/or hole transport auxiliary layer including the organic compound represented by Chemical Formula 1 of the present invention are combined with the light emitting layer of any color.

Effects of the present invention are not limited to the above-described effects, and other effects that are not mentioned will be able to be clearly understood by those skilled in the art from the overall description of the specification.

### MODE FOR INVENTION

The above-described objects, features, and advantages will be described below in detail with reference to the following embodiments, and thus those skilled in the art to which the present invention pertains will be able to easily carry out the technical spirit of the present invention. In describing the present invention, when it is determined that a detailed description of the known technology related to the present invention may unnecessarily obscure the gist of the present invention, a detailed description thereof will be omitted.

In the specification, when terms “including,” “having,” “consisting of,” “arranging,” “providing,” and the like are used, other portions can be added unless “-only” is used. When a component is expressed in the singular, it includes a case in which the component is provided as a plurality of components unless specifically stated otherwise.

In construing a component in the specification, the component is construed as including the margin of error even when there is no separate explicit description.

In the specification, the arrangement of an arbitrary component on an “upper portion (or a lower portion)” of a component or “above (or under)” the component may not only mean that the arbitrary component is disposed in contact with an upper surface (or a lower surface) of the component, but also mean that other components may be interposed between the component and the arbitrary component disposed above (or under) the component.

The term “halo” or “halogen” used herein includes fluorine, chlorine, bromine, and iodine.

The term “alkyl group” used herein indicates both linear alkyl radicals and branched alkyl radicals. Unless otherwise specified, the alkyl group contains 1 to 20 carbon atoms and includes methyl, ethyl, propyl, isopropyl, butyl, isobutyl, tert-butyl, and the like, and additionally, the alkyl group may be substituted arbitrarily.

The term “cycloalkyl group” indicates cyclic alkyl radicals. Unless otherwise specified, the cycloalkyl group contains 3 to 20 carbon atoms and includes cyclopropyl, cyclopentyl, cyclohexyl, and the like, and additionally, the cycloalkyl group may be substituted arbitrarily.

The term “alkenyl group” indicates both linear alkene radicals and branched alkene radicals. Unless otherwise specified, the alkenyl group contains 2 to 20 carbon atoms, and additionally, the alkenyl group may be substituted arbitrarily.

The term “alkynyl group” used herein indicates both linear alkyne radicals and branched alkyne radicals. Unless otherwise specified, the alkynyl group contains 2 to 20 carbon atoms. Additionally, the alkynyl group may be substituted arbitrarily.

The terms “aralkyl group” or “arylalkyl group” used herein are used interchangeably and indicates an alkyl group having an aromatic group as a substituent, and additionally, the aralkyl group (arylalkyl group) may be substituted arbitrarily.

Unless otherwise specified, the term “carbon ring” used herein may be used as the term including both “cycloalkyl group”, which is an alicyclic ring group, and/or “aryl group (aromatic group)”, which is an aromatic ring group.

The term “aryl group” or “aromatic group” used herein is used with the same meaning, and the aryl group includes both single ring groups and polycyclic ring groups. The polycyclic ring may include “condensed ring,” which are two or more rings in which two carbons are common to two adjacent rings. Unless otherwise specified, the aryl group contains 6 to 60 carbon atoms, and additionally, the aryl group may be substituted arbitrarily.

The term “heterocyclic group” used herein indicates that one or more of the carbon atoms constituting the aryl group, the cycloalkyl group, and the aralkyl group (arylalkyl group) are substituted with a heteroatom such as oxygen (O), nitrogen (N), or sulfur (S), and additionally, the heterocycle may be substituted arbitrarily.

The terms “heteroalkyl group” and “heteroalkenyl group” used herein indicate that one or more of the carbon atoms constituting the same are substituted with heteroatoms such as oxygen (O), nitrogen (N), and sulfur (S), and additionally, the heteroalkyl group and the heteroalkenyl group may be substituted arbitrarily.

The term “substituted” used herein indicates that a substituent other than hydrogen (H) is bonded to the corresponding carbon.

Unless otherwise specified herein, a position to be substituted is not limited as long as it is a position where a hydrogen atom is substituted, that is, a position where a substituent may be substituted, and when two or more substituents are present, the substituents may be the same as or different from each other.

The objects and substituents defined in the specification may be the same as or different from each other unless otherwise specified.

### DETAILED DESCRIPTION

Hereinafter, a structure of an organic compound according to the present invention and an organic light emitting diode including the same will be described in detail.

According to one embodiment of the present invention, the organic light emitting diode of the present invention may include a first electrode, a second electrode facing the first electrode, and an organic layer disposed between the first electrode and the second electrode. The organic layer may include a hole injection layer, a hole transport layer, a light emitting layer, an electron transport layer, and an electron injection layer and optionally further include a hole transport auxiliary layer. A surface of a cathode may include a capping layer. Additionally, a seal cap containing a moisture absorbent may be bonded on the capping layer using a UV curable adhesive to form a protecting film (encapsulation layer or protecting layer) to protect the organic light emitting diode from oxygen or moisture in the atmosphere.

According to one embodiment of the present invention, the organic light emitting diode can be used in any one of a flat panel display device, a flexible display device, a monochromatic or white flat panel lighting device, a monochromatic or white flexible lighting device, a vehicle display device, and a display device for virtual or augmented reality, but is not limited thereto.

The first electrode may be an anode and may be made of ITO, IZO, tin-oxide, or zinc-oxide, which are conductive materials with a relatively high work function value, but is not limited thereto.

The second electrode may be a cathode and may include Al, Mg, Ca, Ag, or an alloy or combination thereof, which is a conductive material with a relatively low work function value, but is not limited thereto.

A hole injection layer (HIL) may be formed on the first electrode. A material for the hole injection layer may include HATCN, copper phthalocyanine (CuPc), 4,4′,4″-tris(3-methylphenylamino)triphenylamine (m-MTDATA), 4,4′,4″-tris(3-methylphenylamino)phenoxybenzene (m-MTDAPB), 4,4′,4″-tri(N-carbazolyl)triphenylamine (TCTA), 4,4′,4″-tris(N-(2-naphthyl)-N-phenylamino)-triphenylamine(2-TNATA), and the like, but is not limited thereto.

A hole transport layer (HTL) is disposed adjacent to a light emitting layer between the first electrode and the light emitting layer, and optionally, a hole transport auxiliary layer may be disposed on the hole transport layer, and the hole transport auxiliary layer may be preferably disposed. The hole transport layer and the hole transport auxiliary layer of the present invention should be made of a material with excellent hole transport characteristics, and in particular, a material for forming the hole transport auxiliary layer can minimize a difference in HOMO energy levels between the hole transport layer and the light emitting layer together with the excellent hole transport characteristics, and thus reduce the accumulation of holes at an interface between the hole transport auxiliary layer and the light emitting layer by adjusting the injection characteristics of holes, thereby reducing quenching in which excitons are annihilated by polarons at the interface. Therefore, it is possible to reduce a degradation phenomenon of the device, thereby stabilizing the device and increasing efficiency and lifetime of the device.

To satisfy the above characteristics, in the present invention, an organic compound represented by Chemical Formula 1 of the present invention was derived as a material for a hole transport layer and/or a hole transport auxiliary layer. The present invention was completed by experimentally confirming the excellent effects in which it was possible to increase the luminous efficiency and lifetime of the organic light emitting diode, reduce an operation voltage, and clearly implement a light emitting layer of any color when the organic compound represented by Chemical Formula 1 of the present invention was applied to the hole transport layer and/or the hole transport auxiliary layer.

One or more of the hole transport layer and the hole transport auxiliary layer of the organic light emitting diode of the present invention may each be made of an organic compound represented by Chemical Formula 1 below.

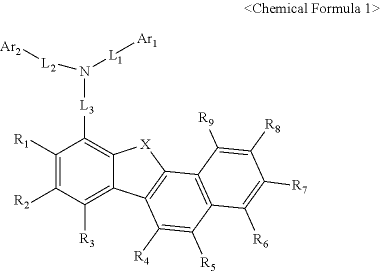

In Chemical Formula 1,

Ar₁ may be any one of Chemical Formulas 2, 3, 4, and 5 below,

X may be O or S.

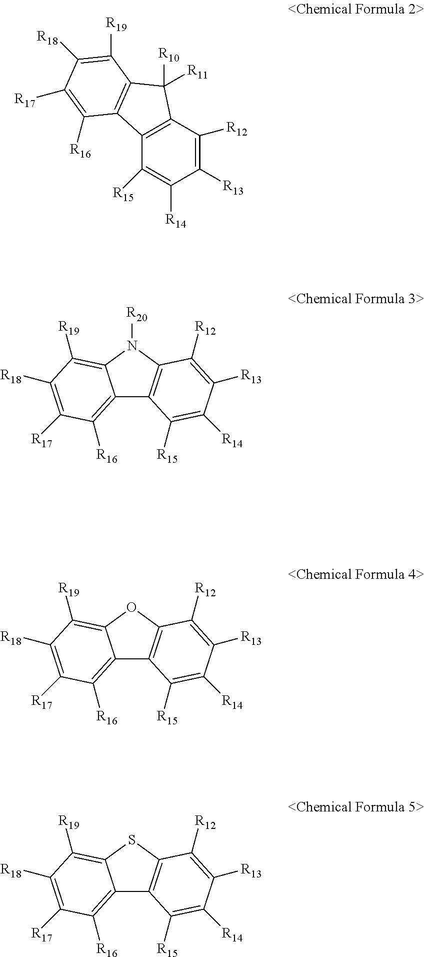

Any one of R₁₅ to R₁₉ of the above Chemical Formulas 2 to 5 may be bonded to L₁ in Chemical Formula 1.

L₁ to L₃ may be the same as or different from each other and be each independently one selected from the group consisting of a single bond, a substituted or unsubstituted aryl group having 6 to 30 carbon atoms, a substituted or unsubstituted aralkyl group having 7 to 30 carbon atoms, a substituted or unsubstituted heteroaryl group having 5 to 60 carbon atoms, and a substituted or unsubstituted heteroarylalkyl group having 6 to 60 carbon atoms.

R₁ to R₂₀ and Ar₂ may be the same as or different from each other and be each independently one selected from the group consisting of hydrogen, deuterium, a cyano group, a nitro group, a halogen, a hydroxy group, a substituted or unsubstituted alkyl group having 1 to 30 carbon atoms, a substituted or unsubstituted alkenyl group having 2 to 30 carbon atoms, a substituted or unsubstituted alkynyl group having 2 to 24 carbon atoms, a substituted or unsubstituted heteroalkyl group having 2 to 30 carbon atoms, a substituted or unsubstituted aryl group having 6 to 30 carbon atoms, a substituted or unsubstituted heteroaryl group having 2 to 30 carbon atoms, a substituted or unsubstituted heteroarylalkyl group having 3 to 30 carbon atoms, a substituted or unsubstituted aralkyl group having 6 to 30 carbon atoms, a substituted or unsubstituted cycloalkyl group having 3 to 20 carbon atoms, a substituted or unsubstituted heterocycloalkyl group having 3 to 20 carbon atoms, a substituted or unsubstituted alkoxy group having 1 to 30 carbon atoms, a substituted or unsubstituted alkylsilyl group having 1 to 30 carbon atoms, a substituted or unsubstituted arylsilyl group having 6 to 30 carbon atoms, and a substituted or unsubstituted aryloxy group having 6 to 30 carbon atoms.

Two adjacent groups among R₁ to R₂₀ may be bonded to form a substituted or unsubstituted carbon ring structure.

Optionally, when L₁ to L₃, R₁ to R₂₀ and Ar₂ are substituted, substituent groups may be one or more selected from the group consisting of deuterium, a cyano group, a nitro group, halogen, a hydroxy group, an alkyl group having 1 to 30 carbon atoms, an alkenyl group having 2 to 30 carbon atoms, an alkynyl group having 2 to 24 carbon atoms, a heteroalkyl group having 2 to 30 carbon atoms, an aralkyl group having 6 to 30 carbon atoms, a cycloalkyl group having 3 to 20 carbon atoms, a heterocycloalkyl group having 3 to 20 carbon atoms, an aryl group having 6 to 30 carbon atoms, a heteroaryl group having 2 to 30 carbon atoms, a heteroarylalkyl group having 3 to 30 carbon atoms, an alkoxy group having 1 to 30 carbon atoms, an alkylsilyl group having 1 to 30 carbon atoms, an arylsilyl group having 6 to 30 carbon atoms, and an aryloxy group having 6 to 30 carbon atoms, and when a plurality of substituents are present, the substituents may be the same as or different from each other.

According to one embodiment of the present invention, Chemical Formula 1 is one in which Ar₁ is selected as Chemical Formula 2 and can be represented by Chemical Formula 1-1 below.

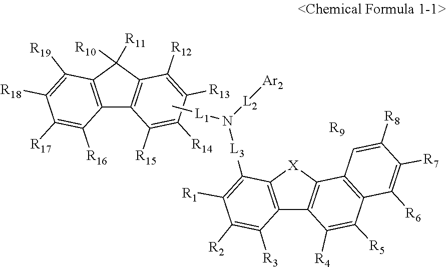

According to one embodiment of the present invention, Chemical Formula 1 may be one represented by any one of Chemical Formulas 6 to 12 below. Chemical Formulas 6 to 9 below are one in which a bonding position of Chemical Formula 2, which is Ari, different in Chemical Formula 1-1, Chemical Formula 10 below is one in which Ar₁ is selected as Chemical Formula 3, Chemical Formula 11 below is one in which Ar₁ is selected as Chemical Formula 4, and Chemical Formula 12 below is one in which Ar₁ is selected as Chemical Formula 5.

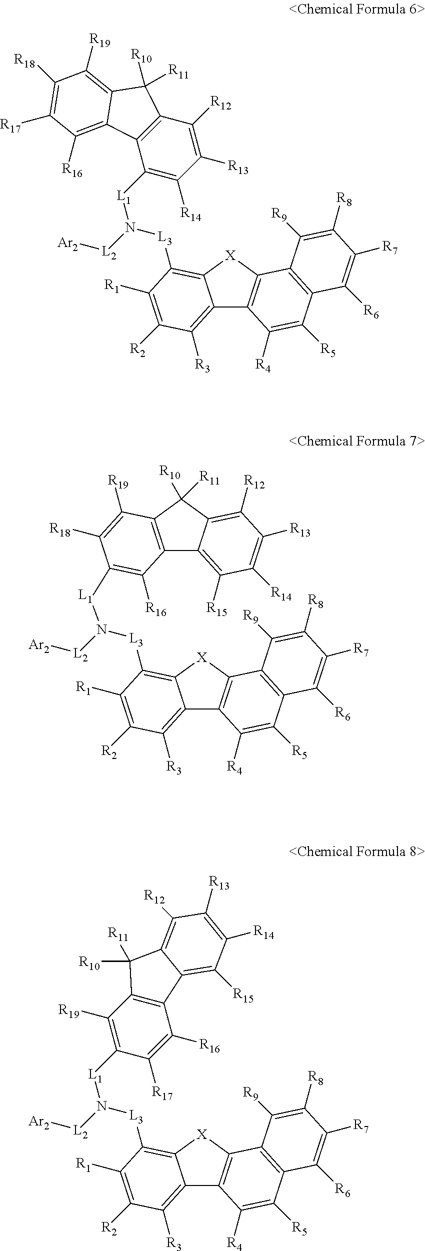

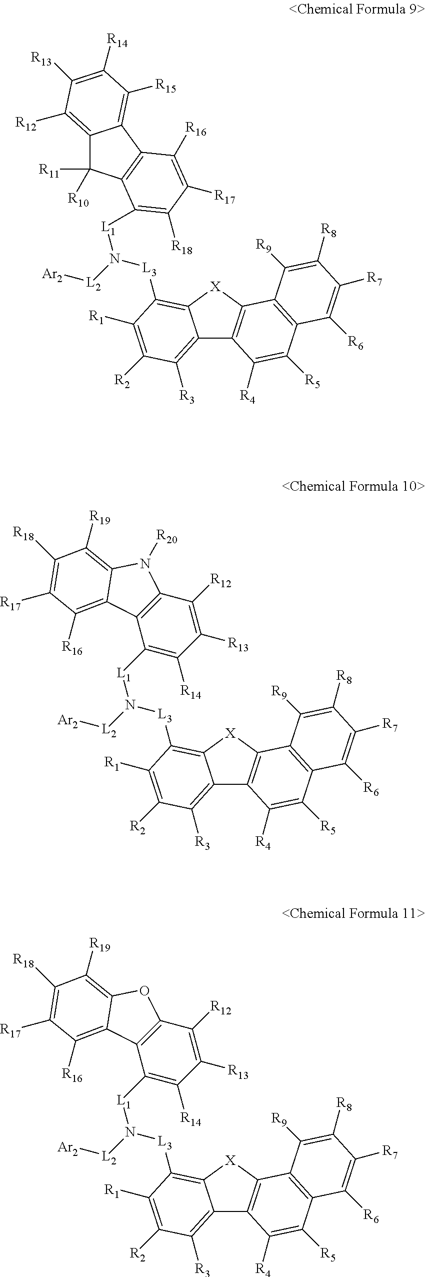

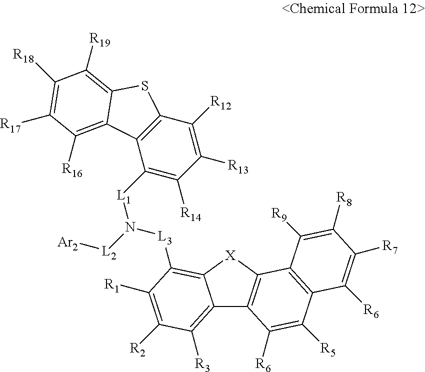

According to one embodiment of the present invention, Ar₂ may be one selected from the group consisting of a substituted or unsubstituted aryl group having 6 to 30 carbon atoms, a substituted or unsubstituted heteroaryl group having 2 to 30 carbon atoms, a substituted or unsubstituted heteroarylalkyl group having 3 to 30 carbon atoms, a substituted or unsubstituted alkyl group having 1 to 30 carbon atoms, and a substituted or unsubstituted cycloalkyl group having 3 to 20 carbon atoms.

According to one embodiment of the present invention, Ar₂ may be one selected from the group consisting of a substituted or unsubstituted phenyl group, a substituted or unsubstituted biphenyl group, a substituted or unsubstituted terphenyl group, a substituted or unsubstituted naphthyl group, a substituted or unsubstituted phenanthrenyl group, a substituted or unsubstituted fluorenyl group, a substituted or unsubstituted spirofluorenyl group, a substituted or unsubstituted dibenzofuranyl group, a substituted or unsubstituted dibenzothiophenyl group, a substituted or unsubstituted carbazolyl group, a substituted or unsubstituted cyclohexyl group, a substituted or unsubstituted napthobenzofuranyl group, and a substituted or unsubstituted napthobenzothiophenyl group.

According to one embodiment of the present invention, L₁ to L₃ may be the same as or different from each other and be each independently one of a single bond or a substituted or unsubstituted phenylene group.

According to one embodiment of the present invention, a substituted or unsubstituted carbon ring structure formed by bonding two adjacent groups among R₁ to R₂₀ may have a monocyclic or polycyclic aryl group structure.

According to one embodiment of the present invention, the compound represented by Chemical Formula 1 may be one selected from the group consisting of Products P1 to P1091 below, but is not limited thereto as long as it is included in the definition of Chemical Formula 1.

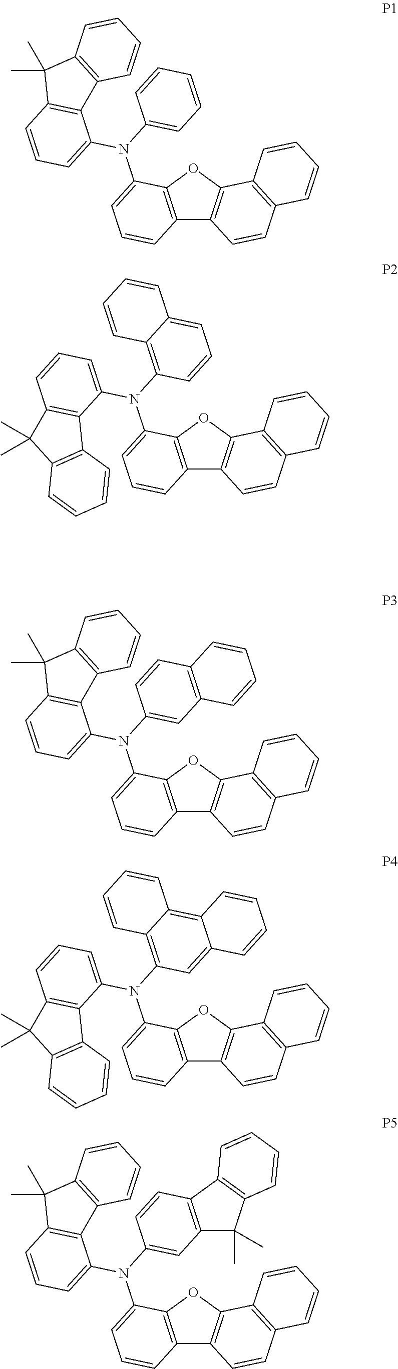

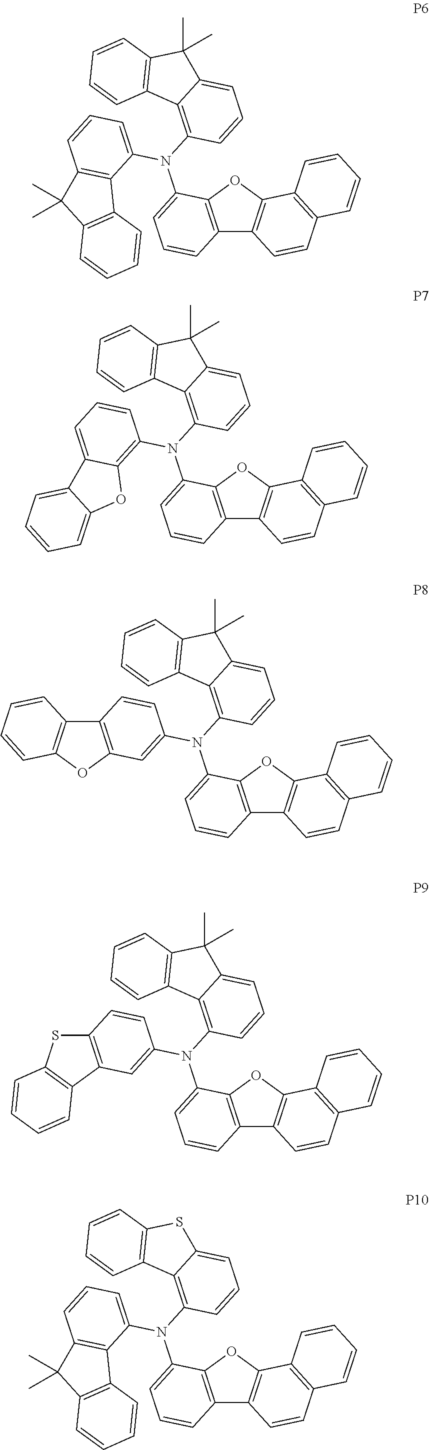 [SMILES: CC1(C)c2ccccc2-c2c(N(c3cccc4c3-c3ccccc3C4(C)C)c3cccc4c3oc3c5ccccc5ccc43)cccc21 ; CC1(C)c2ccccc2-c2c(N(c3cccc4c3oc3ccccc34)c3cccc4c3oc3c5ccccc5ccc43)cccc21 ; CC1(C)c2ccccc2-c2c(N(c3ccc4c(c3)oc3ccccc34)c3cccc4c3oc3c5ccccc5ccc43)cccc21 ; CC1(C)c2ccccc2-c2c(N(c3ccc4sc5ccccc5c4c3)c3cccc4c3oc3c5ccccc5ccc43)cccc21 ; CC1(C)c2ccccc2-c2c(N(c3cccc4c3oc3c5ccccc5ccc43)c3cccc4sc5ccccc5c34)cccc21]

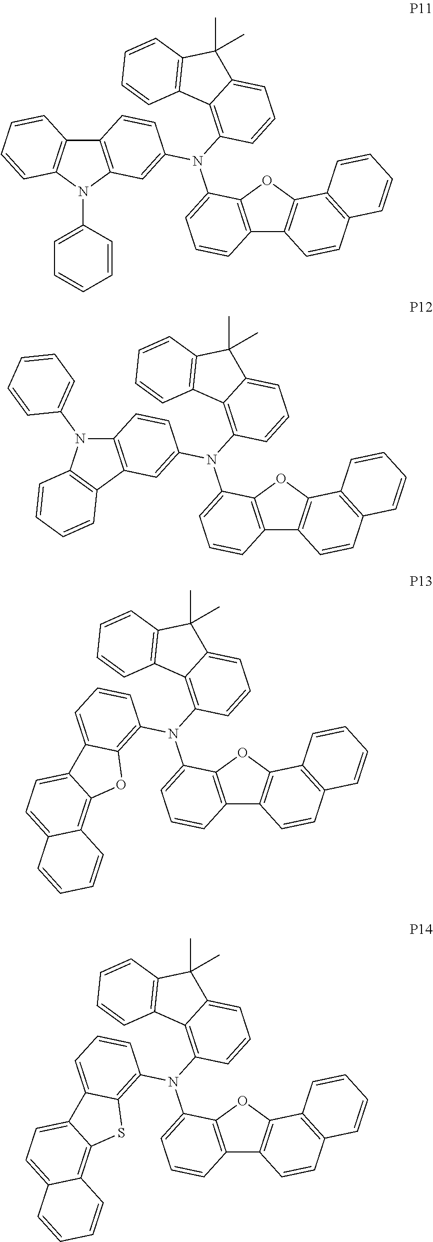

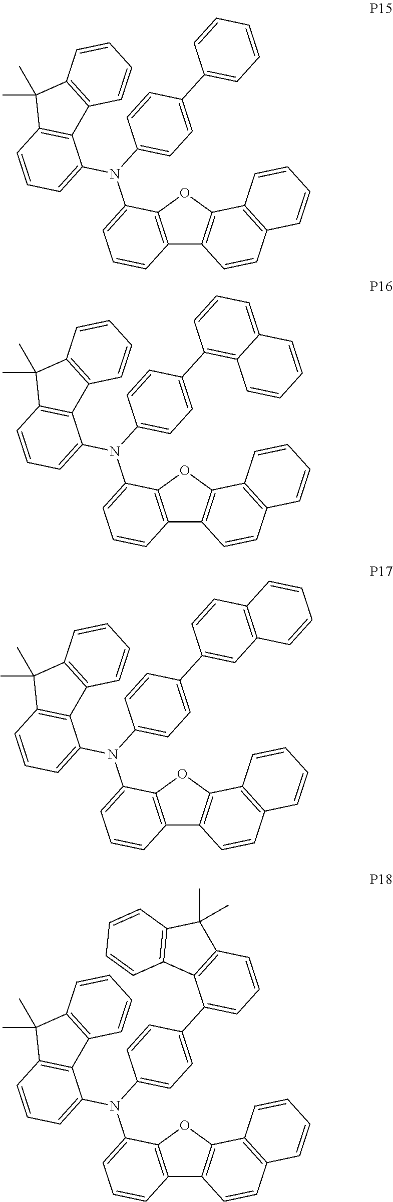

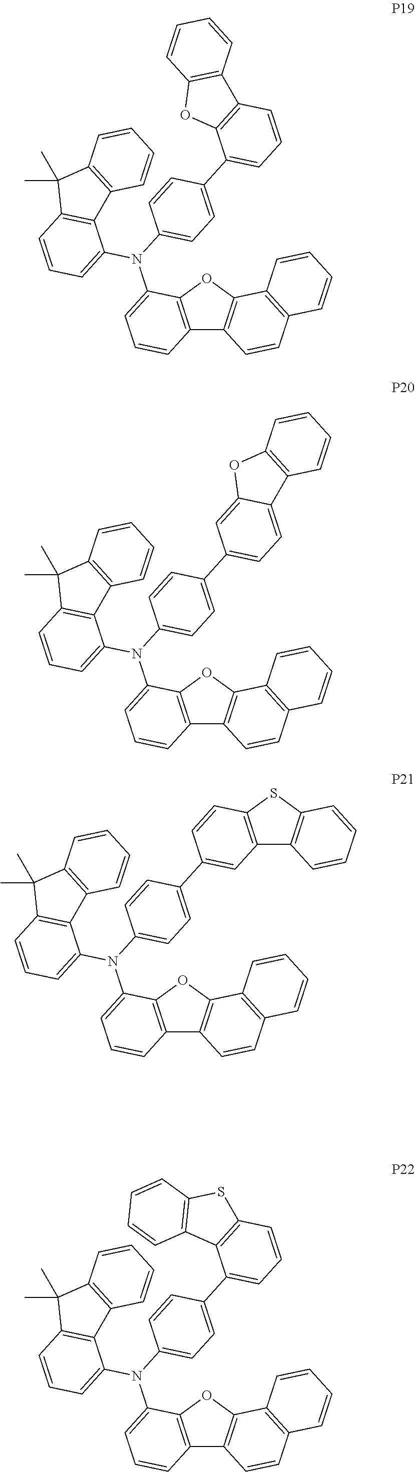 [SMILES: CC1(C)c2ccccc2-c2c(N(c3ccc(-c4cccc5c4oc4ccccc45)cc3)c3cccc4c3oc3c5ccccc5ccc43)cccc21 ; CC1(C)c2ccccc2-c2c(N(c3ccc(-c4ccc5c(c4)oc4ccccc45)cc3)c3cccc4c3oc3c5ccccc5ccc43)cccc21 ; CC1(C)c2ccccc2-c2c(N(c3ccc(-c4ccc5sc6ccccc6c5c4)cc3)c3cccc4c3oc3c5ccccc5ccc43)cccc21 ; CC1(C)c2ccccc2-c2c(N(c3ccc(-c4cccc5sc6ccccc6c45)cc3)c3cccc4c3oc3c5ccccc5ccc43)cccc21]

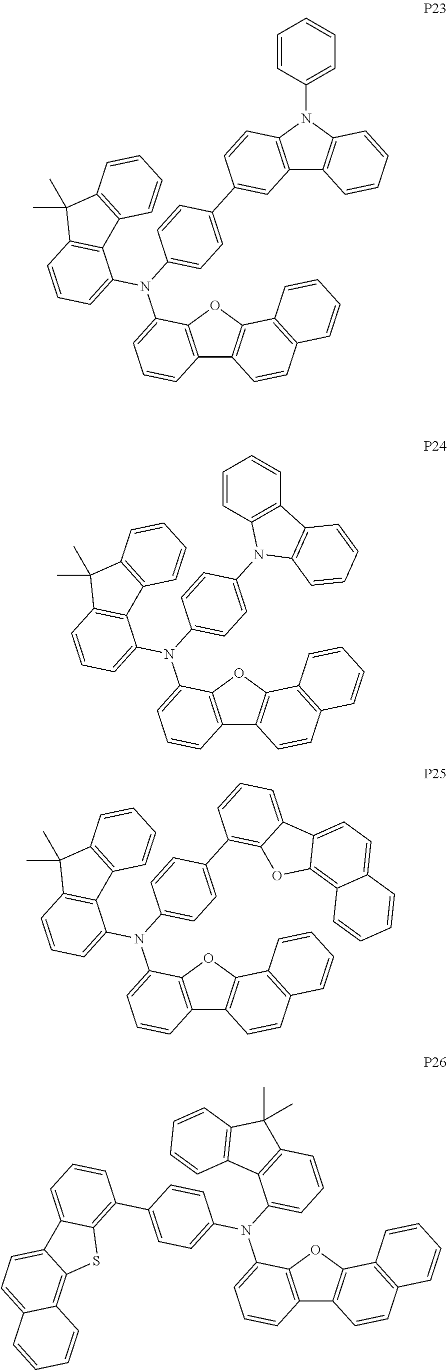

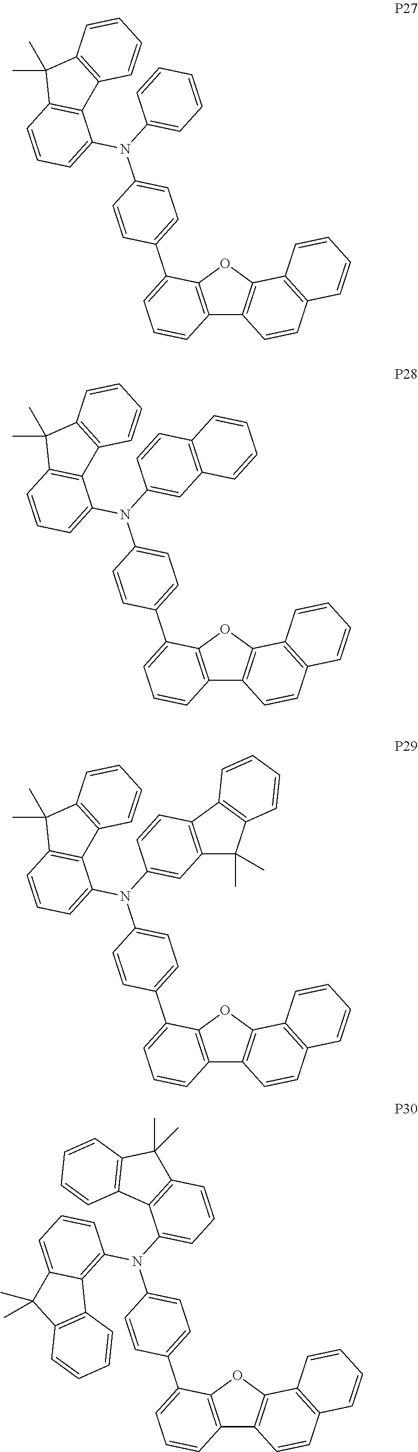

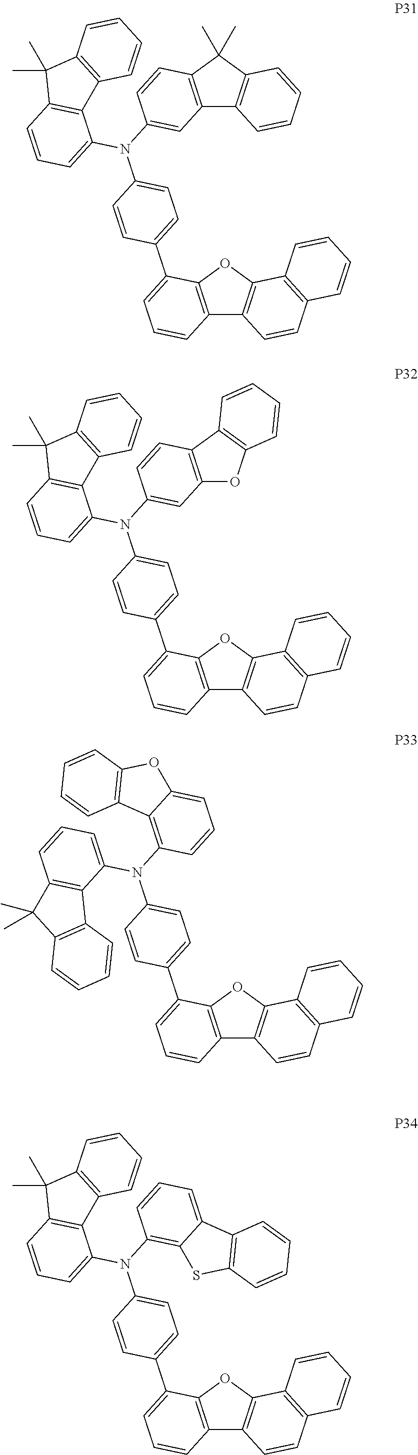

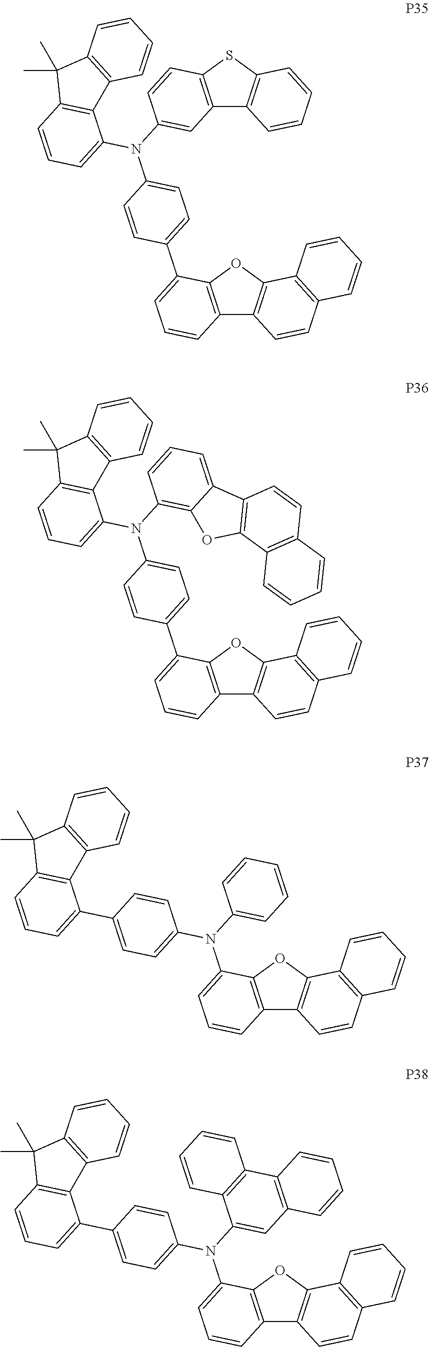 [SMILES: CC1(C)c2ccccc2-c2c(N(c3ccc(-c4cccc5c4oc4c6ccccc6ccc54)cc3)c3ccc4sc5ccccc5c4c3)cccc21 ; CC1(C)c2ccccc2-c2c(N(c3ccc(-c4cccc5c4oc4c6ccccc6ccc54)cc3)c3cccc4c3oc3c5ccccc5ccc43)cccc21 ; CC1(C)c2ccccc2-c2c(-c3ccc(N(c4ccccc4)c4cccc5c4oc4c6ccccc6ccc54)cc3)cccc21 ; CC1(C)c2ccccc2-c2c(-c3ccc(N(c4cc5ccccc5c5ccccc45)c4cccc5c4oc4c6ccccc6ccc54)cc3)cccc21]

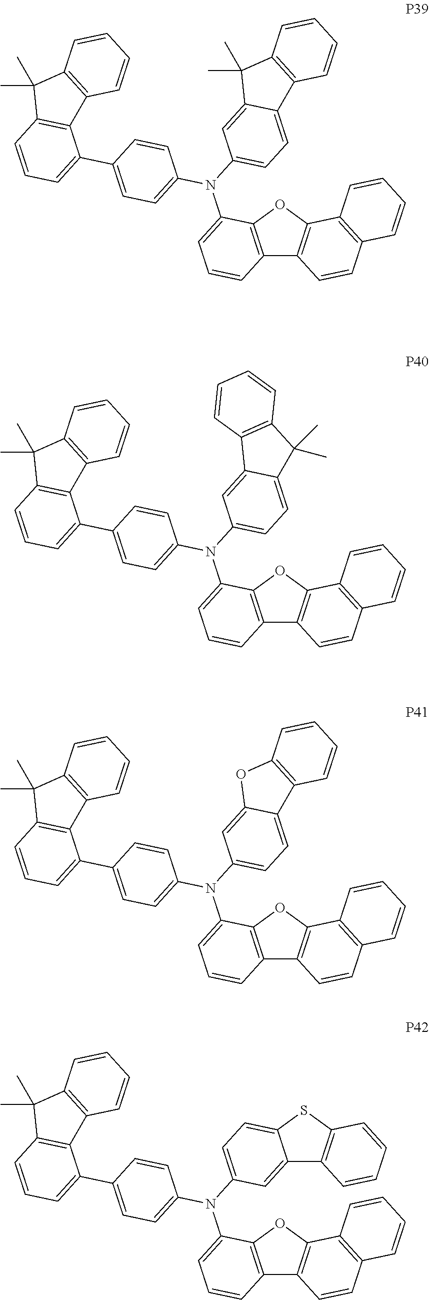 [SMILES: CC1(C)c2ccccc2-c2ccc(N(c3ccc(-c4cccc5c4-c4ccccc4C5(C)C)cc3)c3cccc4c3oc3c5ccccc5ccc43)cc21 ; CC1(C)c2ccccc2-c2cc(N(c3ccc(-c4cccc5c4-c4ccccc4C5(C)C)cc3)c3cccc4c3oc3c5ccccc5ccc43)ccc21 ; CC1(C)c2ccccc2-c2c(-c3ccc(N(c4ccc5c(c4)oc4ccccc45)c4cccc5c4oc4c6ccccc6ccc54)cc3)cccc21 ; CC1(C)c2ccccc2-c2c(-c3ccc(N(c4ccc5sc6ccccc6c5c4)c4cccc5c4oc4c6ccccc6ccc54)cc3)cccc21]

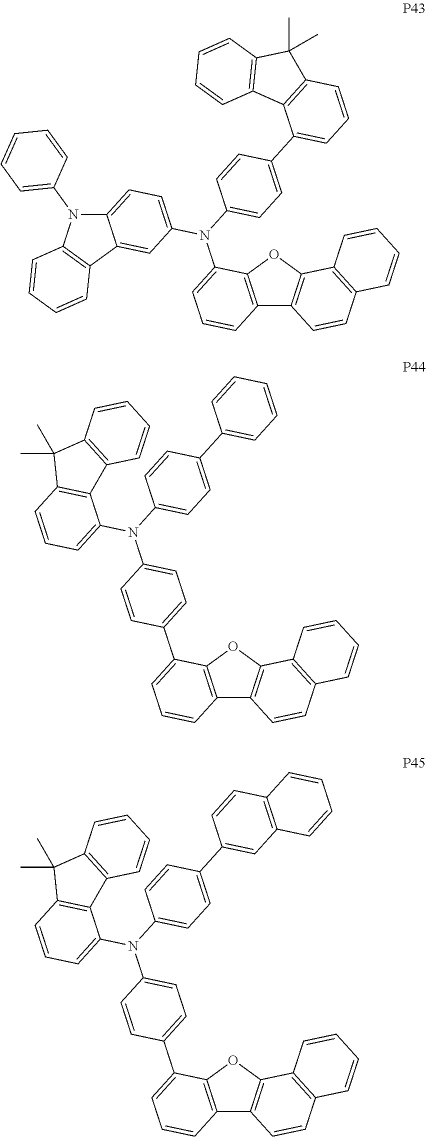

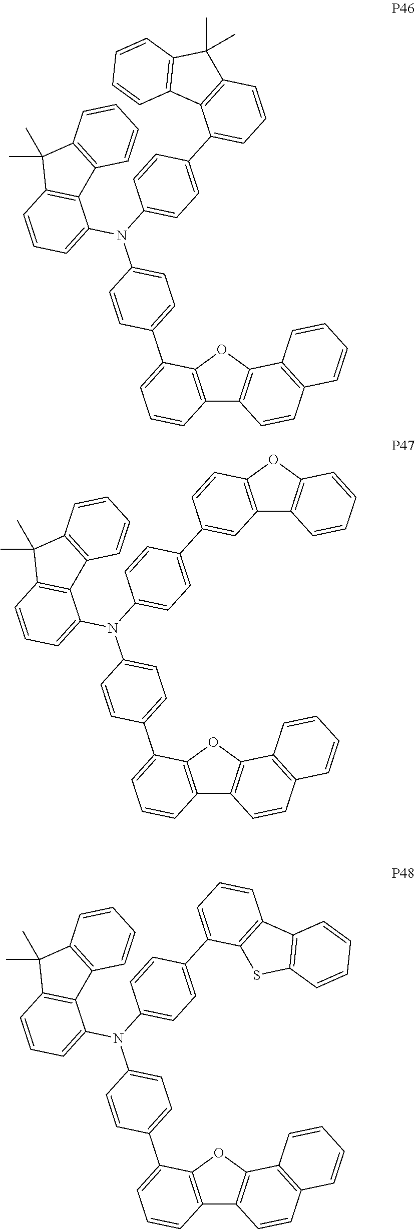

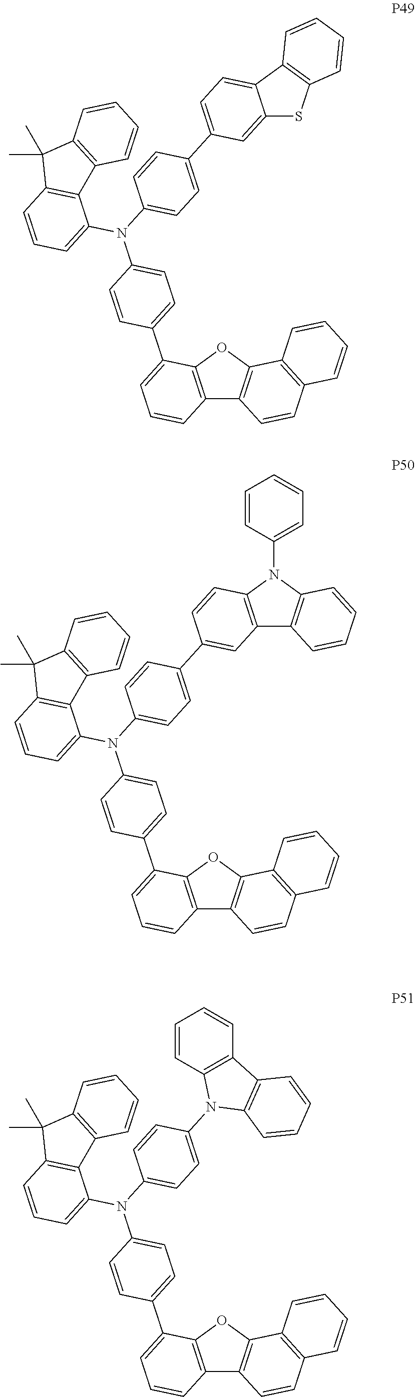 [SMILES: CC1(C)c2ccccc2-c2c(N(c3ccc(-c4ccc5c(c4)sc4ccccc45)cc3)c3ccc(-c4cccc5c4oc4c6ccccc6ccc54)cc3)cccc21 ; CC1(C)c2ccccc2-c2c(N(c3ccc(-c4ccc5c(c4)c4ccccc4n5-c4ccccc4)cc3)c3ccc(-c4cccc5c4oc4c6ccccc6ccc54)cc3)cccc21 ; CC1(C)c2ccccc2-c2c(N(c3ccc(-c4cccc5c4oc4c6ccccc6ccc54)cc3)c3ccc(-n4c5ccccc5c5ccccc54)cc3)cccc21]

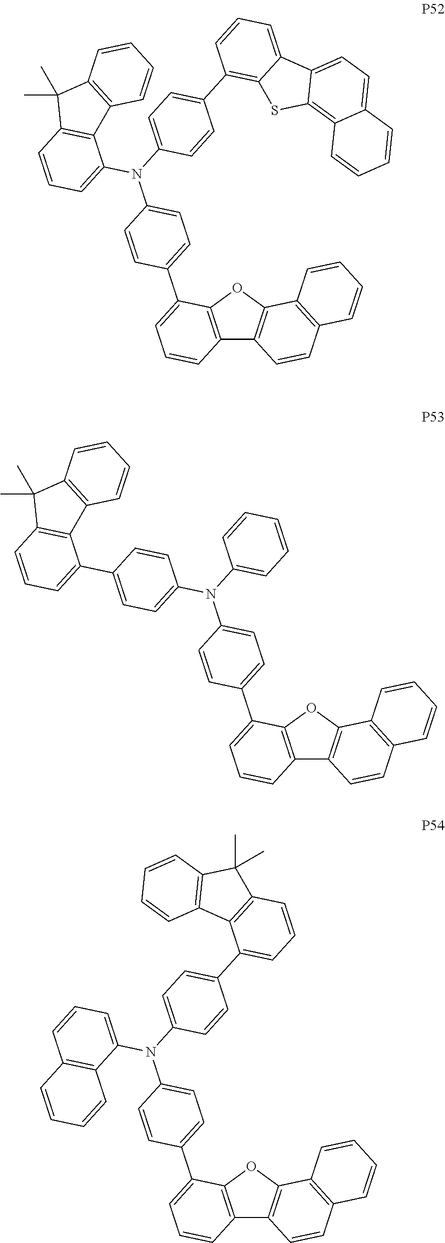 [SMILES: CC1(C)c2ccccc2-c2c(N(c3ccc(-c4cccc5c4oc4c6ccccc6ccc54)cc3)c3ccc(-c4cccc5c4sc4c6ccccc6ccc54)cc3)cccc21 ; CC1(C)c2ccccc2-c2c(-c3ccc(N(c4ccccc4)c4ccc(-c5cccc6c5oc5c7ccccc7ccc65)cc4)cc3)cccc21 ; CC1(C)c2ccccc2-c2c(-c3ccc(N(c4ccc(-c5cccc6c5oc5c7ccccc7ccc65)cc4)c4cccc5ccccc45)cc3)cccc21]

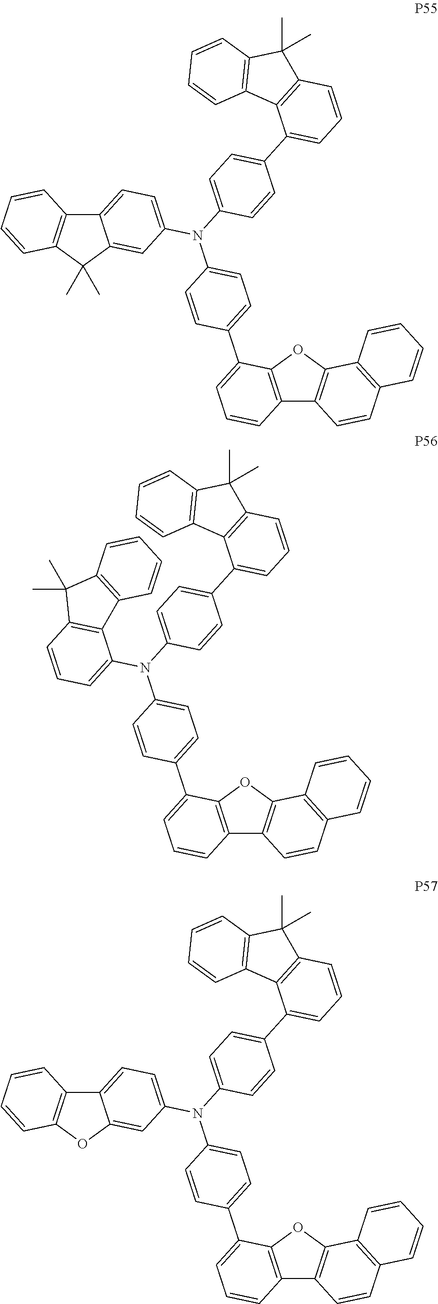

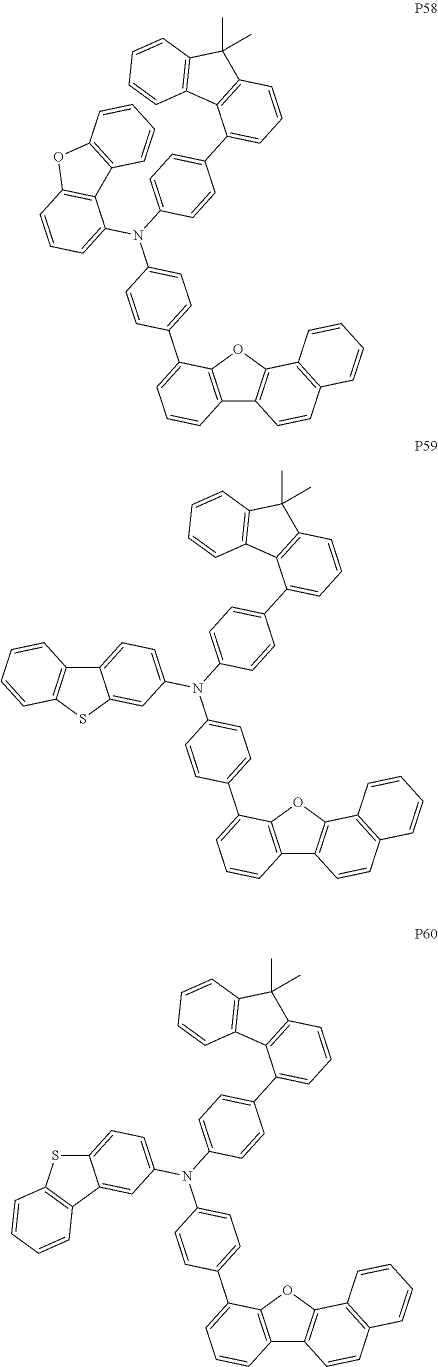

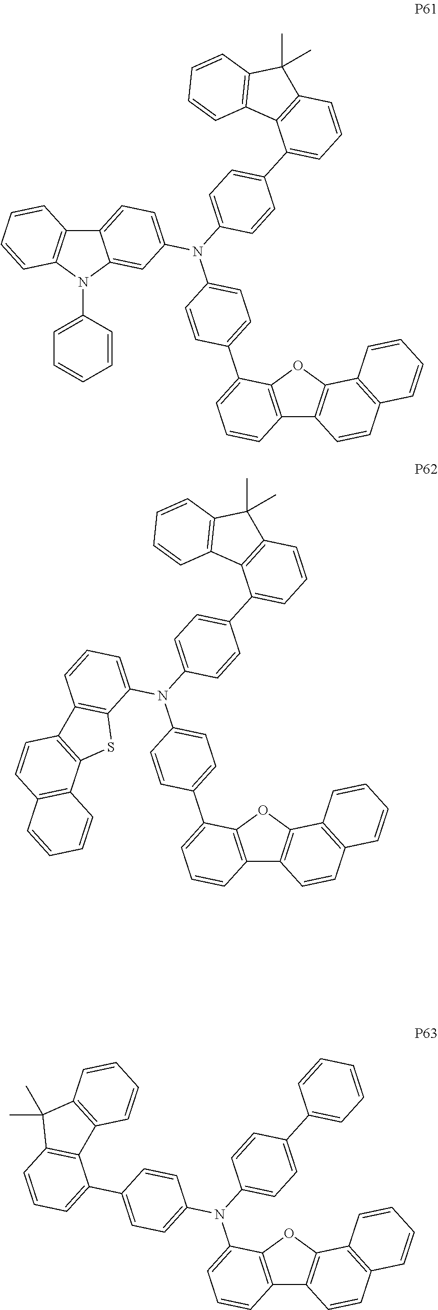

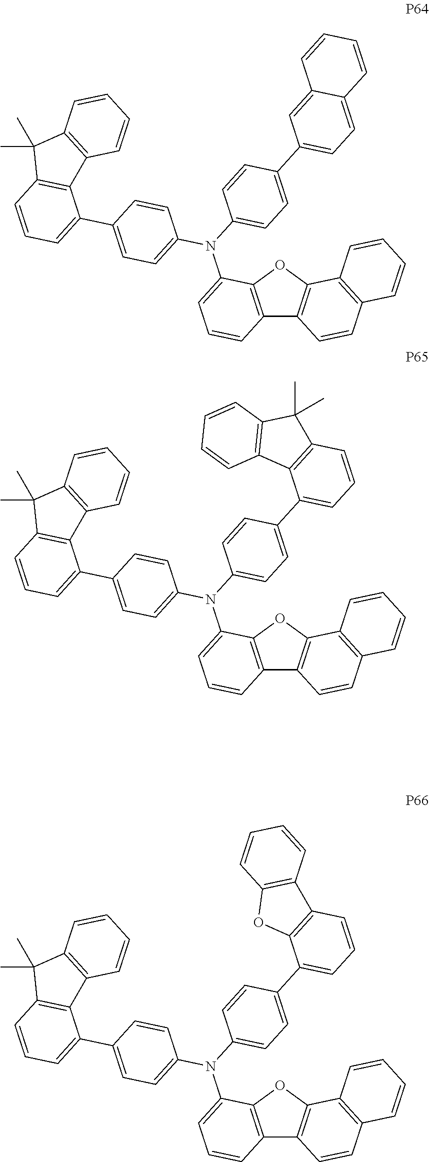 [SMILES: CC1(C)c2ccccc2-c2c(-c3ccc(N(c4ccc(-c5ccc6ccccc6c5)cc4)c4cccc5c4oc4c6ccccc6ccc54)cc3)cccc21 ; CC1(C)c2ccccc2-c2c(-c3ccc(N(c4ccc(-c5cccc6c5-c5ccccc5C6(C)C)cc4)c4cccc5c4oc4c6ccccc6ccc54)cc3)cccc21 ; CC1(C)c2ccccc2-c2c(-c3ccc(N(c4ccc(-c5cccc6c5oc5ccccc56)cc4)c4cccc5c4oc4c6ccccc6ccc54)cc3)cccc21]

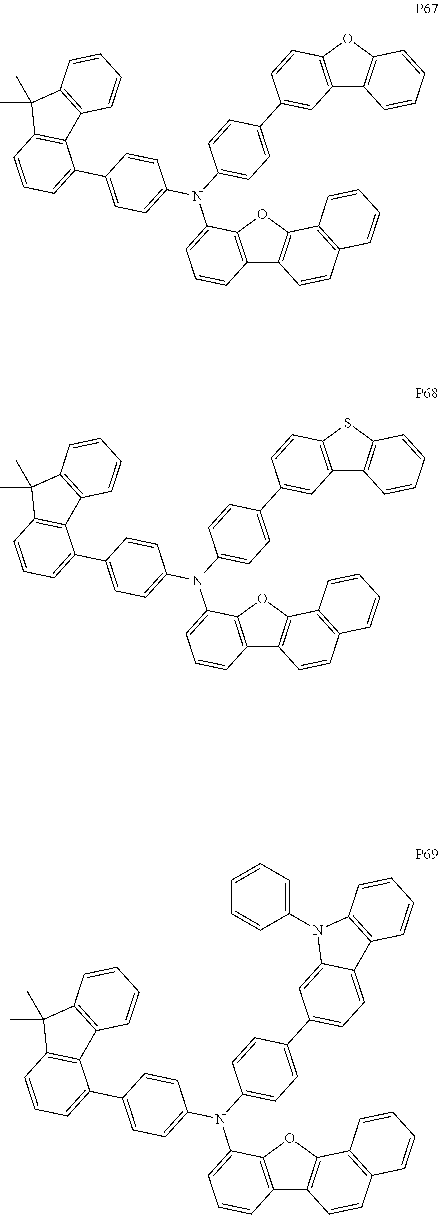 [SMILES: CC1(C)c2ccccc2-c2c(-c3ccc(N(c4ccc(-c5ccc6oc7ccccc7c6c5)cc4)c4cccc5c4oc4c6ccccc6ccc54)cc3)cccc21 ; CC1(C)c2ccccc2-c2c(-c3ccc(N(c4ccc(-c5ccc6sc7ccccc7c6c5)cc4)c4cccc5c4oc4c6ccccc6ccc54)cc3)cccc21 ; CC1(C)c2ccccc2-c2c(-c3ccc(N(c4ccc(-c5ccc6c7ccccc7n(-c7ccccc7)c6c5)cc4)c4cccc5c4oc4c6ccccc6ccc54)cc3)cccc21]

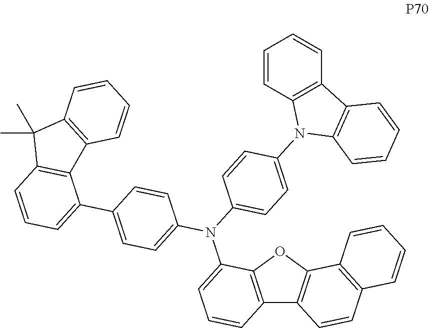 [SMILES: CC1(C)c2ccccc2-c2c(-c3ccc(N(c4ccc(-n5c6ccccc6c6ccccc65)cc4)c4cccc5c4oc4c6ccccc6ccc54)cc3)cccc21]

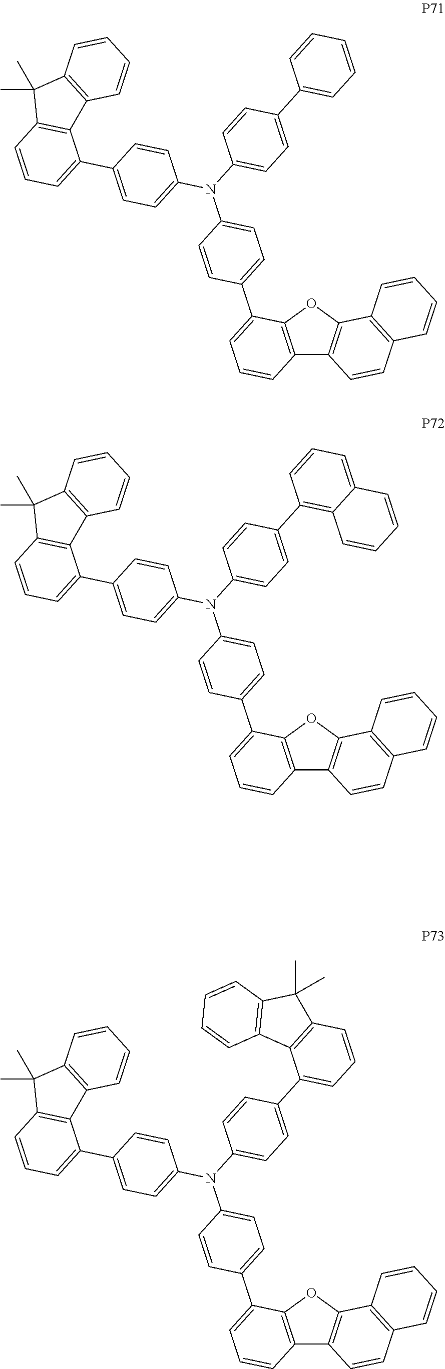 [SMILES: CC1(C)c2ccccc2-c2c(-c3ccc(N(c4ccc(-c5ccccc5)cc4)c4ccc(-c5cccc6c5oc5c7ccccc7ccc65)cc4)cc3)cccc21 ; CC1(C)c2ccccc2-c2c(-c3ccc(N(c4ccc(-c5cccc6ccccc56)cc4)c4ccc(-c5cccc6c5oc5c7ccccc7ccc65)cc4)cc3)cccc21 ; CC1(C)c2ccccc2-c2c(-c3ccc(N(c4ccc(-c5cccc6c5-c5ccccc5C6(C)C)cc4)c4ccc(-c5cccc6c5oc5c7ccccc7ccc65)cc4)cc3)cccc21]

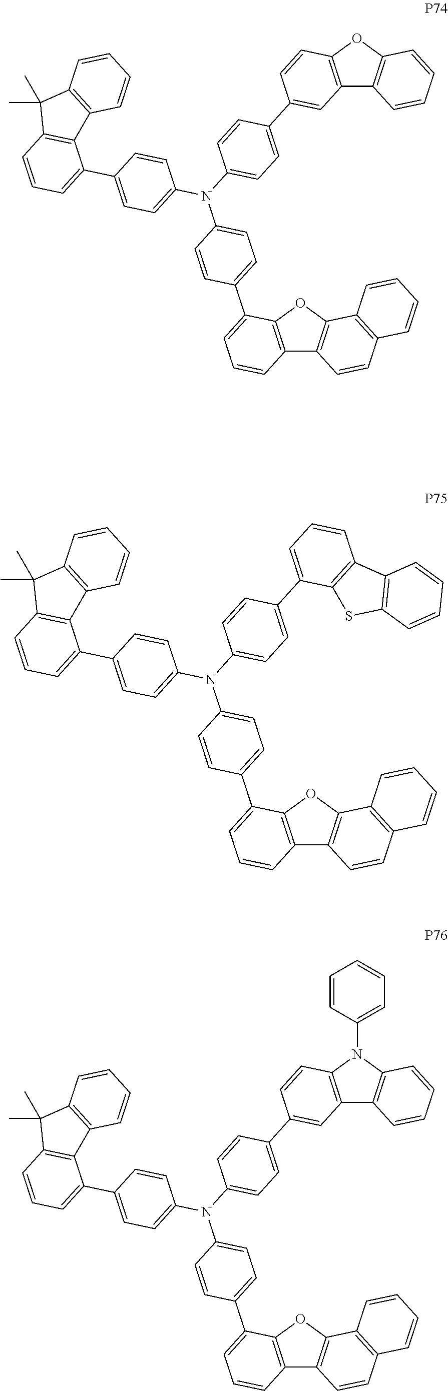 [SMILES: CC1(C)c2ccccc2-c2c(-c3ccc(N(c4ccc(-c5ccc6oc7ccccc7c6c5)cc4)c4ccc(-c5cccc6c5oc5c7ccccc7ccc65)cc4)cc3)cccc21 ; CC1(C)c2ccccc2-c2c(-c3ccc(N(c4ccc(-c5cccc6c5oc5c7ccccc7ccc65)cc4)c4ccc(-c5cccc6c5sc5ccccc56)cc4)cc3)cccc21 ; CC1(C)c2ccccc2-c2c(-c3ccc(N(c4ccc(-c5ccc6c(c5)c5ccccc5n6-c5ccccc5)cc4)c4ccc(-c5cccc6c5oc5c7ccccc7ccc65)cc4)cc3)cccc21]

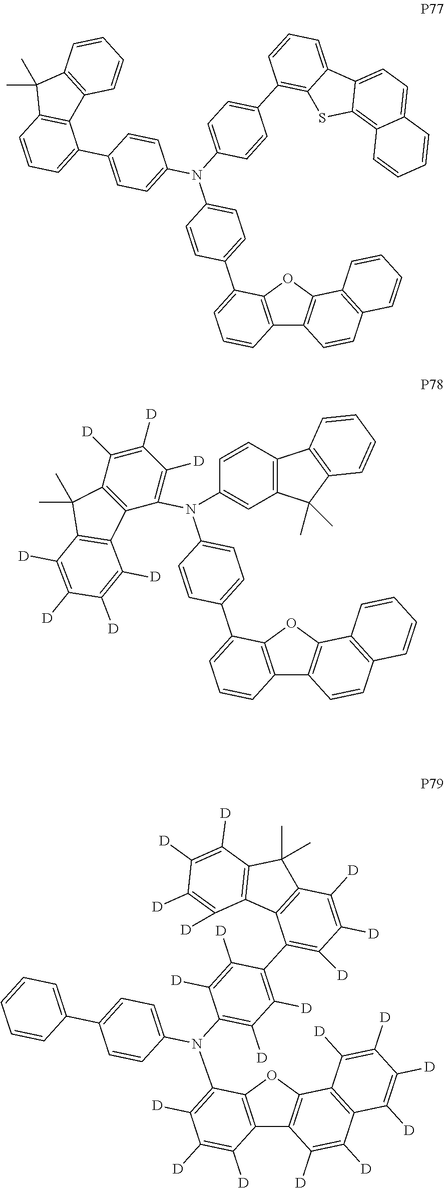 [SMILES: CC1(C)c2ccccc2-c2c(-c3ccc(N(c4ccc(-c5cccc6c5oc5c7ccccc7ccc65)cc4)c4ccc(-c5cccc6c5sc5c7ccccc7ccc65)cc4)cc3)cccc21 ; [2H]c1c([2H])c([2H])c2c(c1[2H])-c1c(N(c3ccc(-c4cccc5c4oc4c6ccccc6ccc54)cc3)c3ccc4c(c3)C(C)(C)c3ccccc3-4)c([2H])c([2H])c([2H])c1C2(C)C ; [2H]c1c([2H])c([2H])c2c(c1[2H])-c1c(-c3c([2H])c([2H])c(N(c4ccc(-c5ccccc5)cc4)c4c([2H])c([2H])c([2H])c5c4oc4c6c([2H])c([2H])c([2H])c([2H])c6c([2H])c([2H])c45)c([2H])c3[2H])c([2H])c([2H])c([2H])c1C2(C)C]

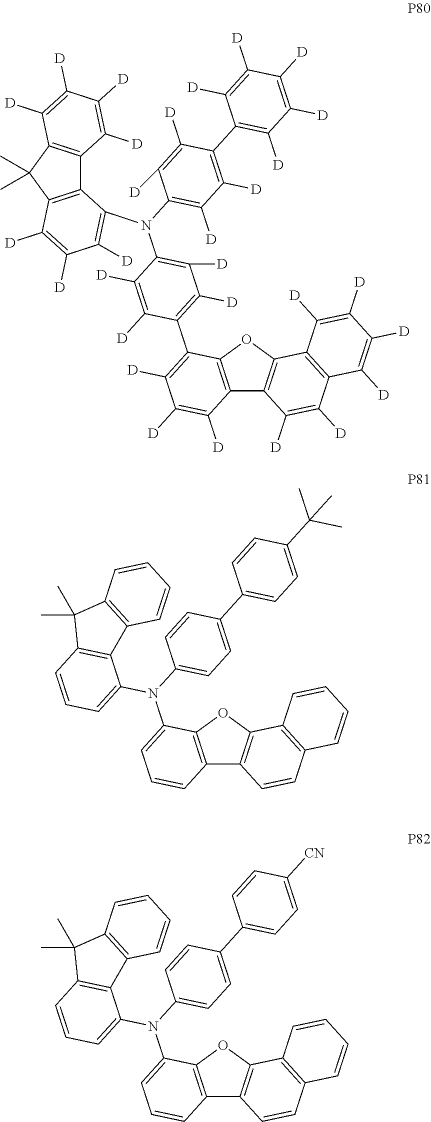 [SMILES: [2H]c1c([2H])c([2H])c(-c2c([2H])c([2H])c(N(c3c([2H])c([2H])c(-c4c([2H])c([2H])c([2H])c5c4oc4c6c([2H])c([2H])c([2H])c([2H])c6c([2H])c([2H])c45)c([2H])c3[2H])c3c([2H])c([2H])c([2H])c4c3-c3c([2H])c([2H])c([2H])c([2H])c3C4(C)C)c([2H])c2[2H])c([2H])c1[2H] ; CC(C)(C)c1ccc(-c2ccc(N(c3cccc4c3-c3ccccc3C4(C)C)c3cccc4c3oc3c5ccccc5ccc43)cc2)cc1 ; CC1(C)c2ccccc2-c2c(N(c3ccc(-c4ccc(C#N)cc4)cc3)c3cccc4c3oc3c5ccccc5ccc43)cccc21]

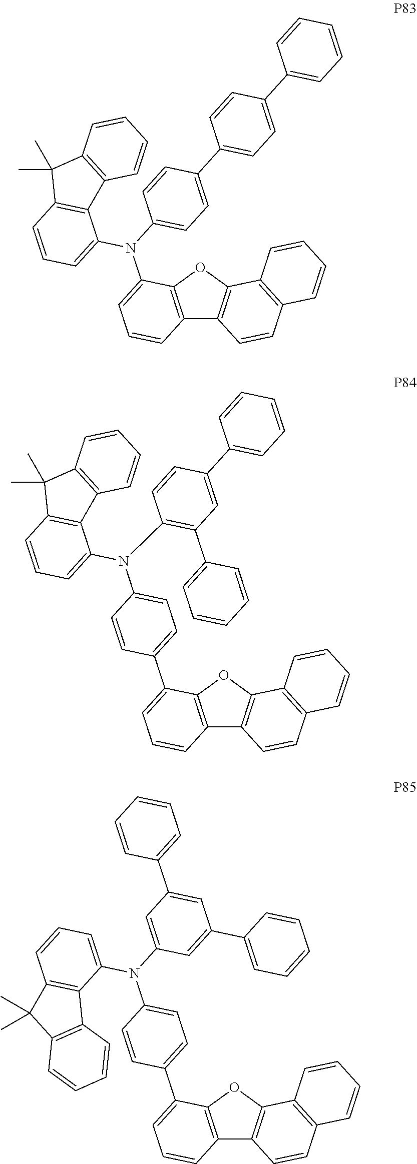 [SMILES: CC1(C)c2ccccc2-c2c(N(c3ccc(-c4ccc(-c5ccccc5)cc4)cc3)c3cccc4c3oc3c5ccccc5ccc43)cccc21 ; CC1(C)c2ccccc2-c2c(N(c3ccc(-c4cccc5c4oc4c6ccccc6ccc54)cc3)c3ccc(-c4ccccc4)cc3-c3ccccc3)cccc21 ; CC1(C)c2ccccc2-c2c(N(c3ccc(-c4cccc5c4oc4c6ccccc6ccc54)cc3)c3cc(-c4ccccc4)cc(-c4ccccc4)c3)cccc21]

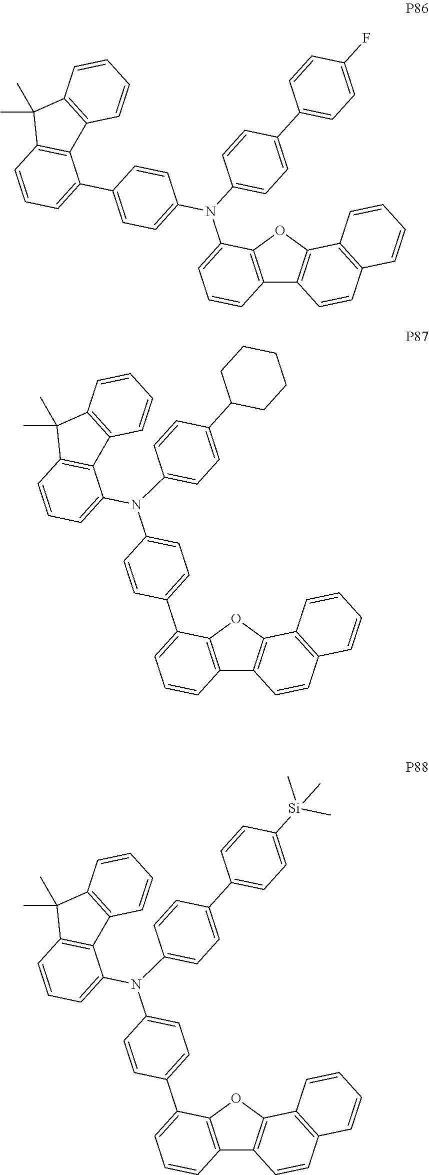

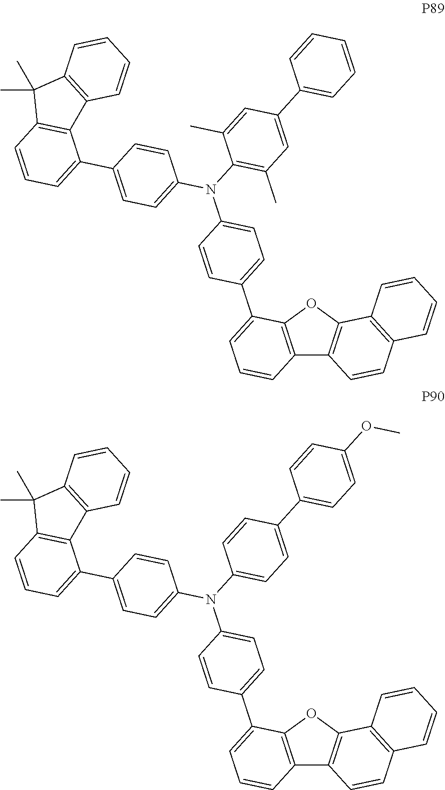 [SMILES: Cc1cc(-c2ccccc2)cc(C)c1N(c1ccc(-c2cccc3c2-c2ccccc2C3(C)C)cc1)c1ccc(-c2cccc3c2oc2c4ccccc4ccc32)cc1 ; COc1ccc(-c2ccc(N(c3ccc(-c4cccc5c4-c4ccccc4C5(C)C)cc3)c3ccc(-c4cccc5c4oc4c6ccccc6ccc54)cc3)cc2)cc1]

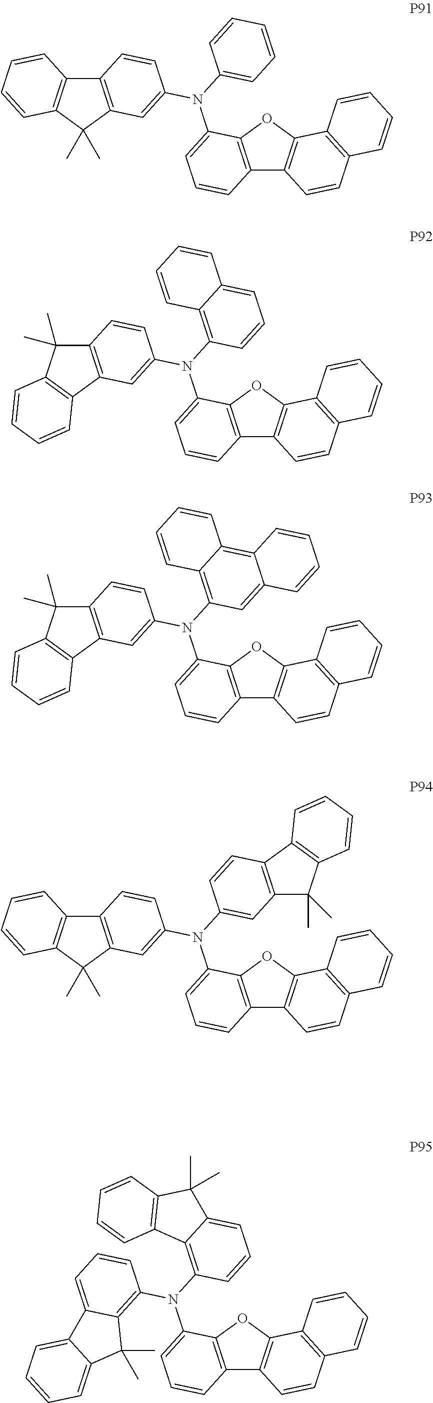 [SMILES: CC1(C)c2ccccc2-c2ccc(N(c3ccccc3)c3cccc4c3oc3c5ccccc5ccc43)cc21 ; CC1(C)c2ccccc2-c2cc(N(c3cccc4ccccc34)c3cccc4c3oc3c5ccccc5ccc43)ccc21 ; CC1(C)c2ccccc2-c2cc(N(c3cc4ccccc4c4ccccc34)c3cccc4c3oc3c5ccccc5ccc43)ccc21 ; CC1(C)c2ccccc2-c2ccc(N(c3ccc4c(c3)C(C)(C)c3ccccc3-4)c3cccc4c3oc3c5ccccc5ccc43)cc21 ; CC1(C)c2ccccc2-c2c(N(c3cccc4c3C(C)(C)c3ccccc3-4)c3cccc4c3oc3c5ccccc5ccc43)cccc21]

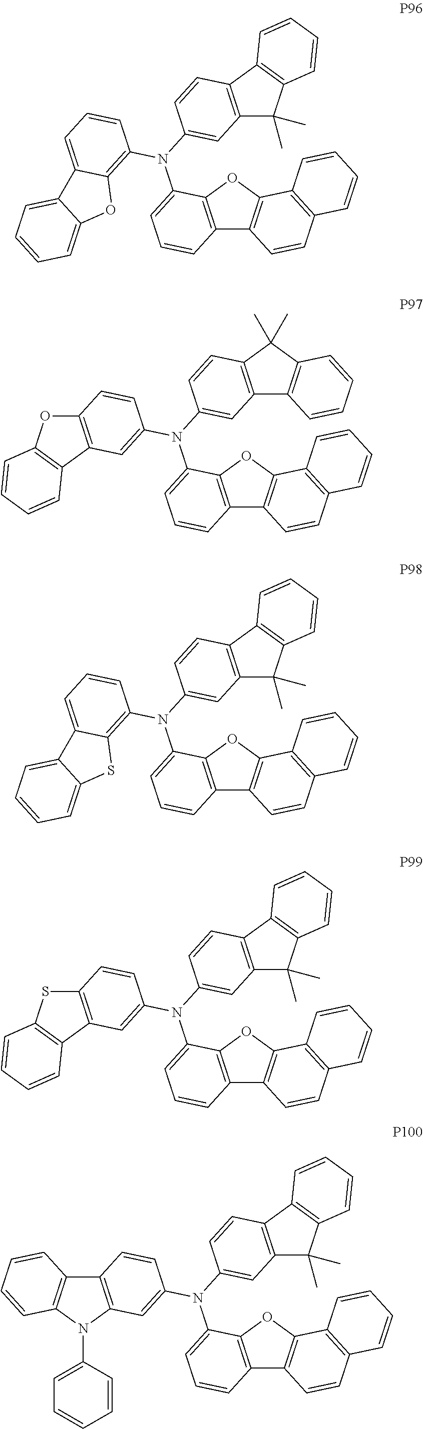 [SMILES: CC1(C)c2ccccc2-c2ccc(N(c3cccc4c3oc3ccccc34)c3cccc4c3oc3c5ccccc5ccc43)cc21 ; CC1(C)c2ccccc2-c2cc(N(c3ccc4oc5ccccc5c4c3)c3cccc4c3oc3c5ccccc5ccc43)ccc21 ; CC1(C)c2ccccc2-c2ccc(N(c3cccc4c3oc3c5ccccc5ccc43)c3cccc4c3sc3ccccc34)cc21 ; CC1(C)c2ccccc2-c2ccc(N(c3ccc4sc5ccccc5c4c3)c3cccc4c3oc3c5ccccc5ccc43)cc21 ; CC1(C)c2ccccc2-c2ccc(N(c3ccc4c5ccccc5n(-c5ccccc5)c4c3)c3cccc4c3oc3c5ccccc5ccc43)cc21]

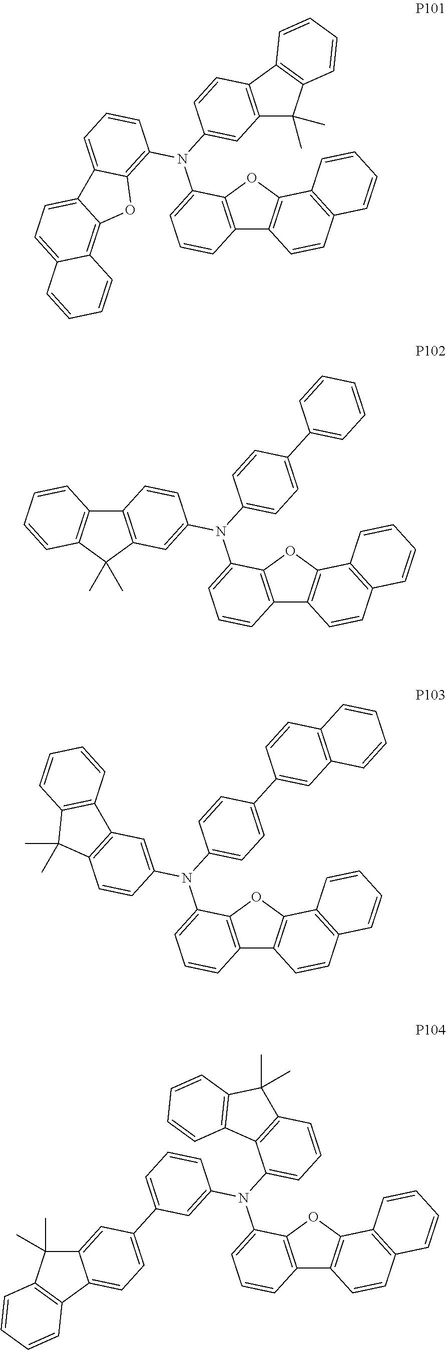

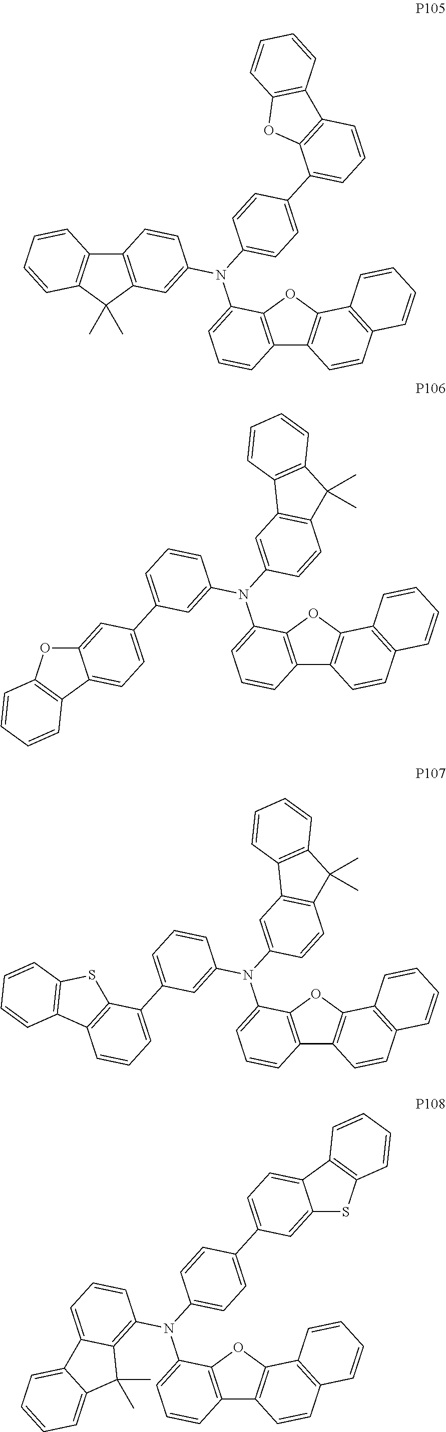

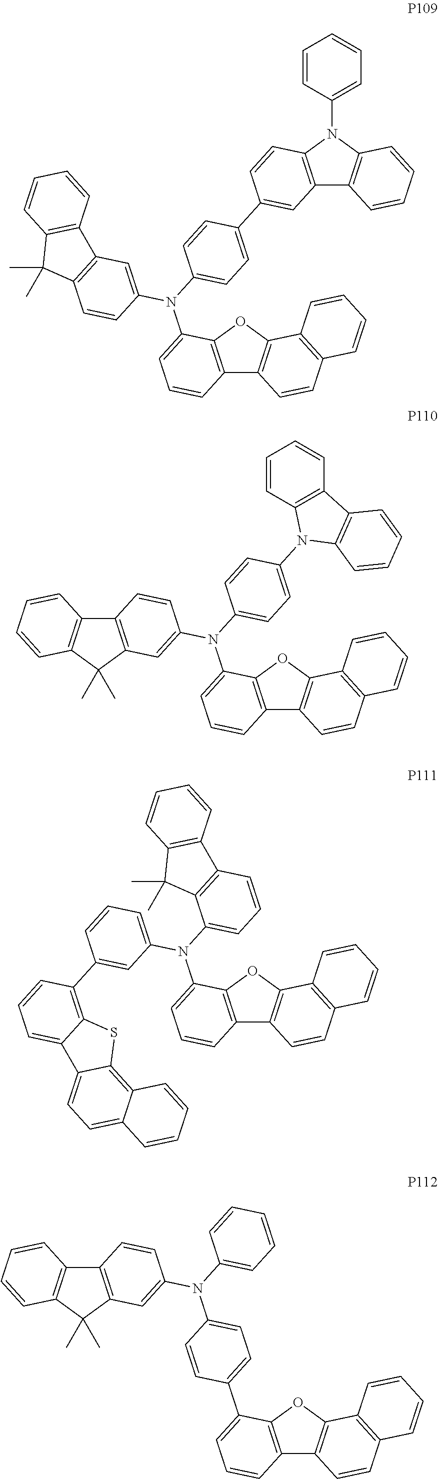

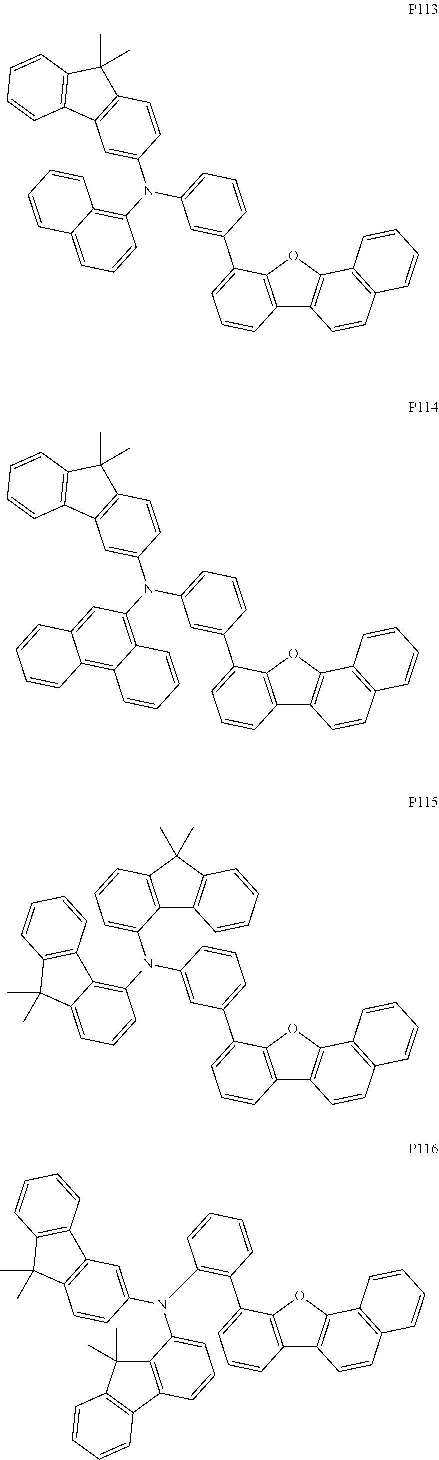 [SMILES: CC1(C)c2ccccc2-c2cc(N(c3cccc(-c4cccc5c4oc4c6ccccc6ccc54)c3)c3cccc4ccccc34)ccc21 ; CC1(C)c2ccccc2-c2cc(N(c3cccc(-c4cccc5c4oc4c6ccccc6ccc54)c3)c3cc4ccccc4c4ccccc34)ccc21 ; CC1(C)c2ccccc2-c2c(N(c3cccc(-c4cccc5c4oc4c6ccccc6ccc54)c3)c3cccc4c3-c3ccccc3C4(C)C)cccc21 ; CC1(C)c2ccccc2-c2cc(N(c3ccccc3-c3cccc4c3oc3c5ccccc5ccc43)c3cccc4c3C(C)(C)c3ccccc3-4)ccc21]

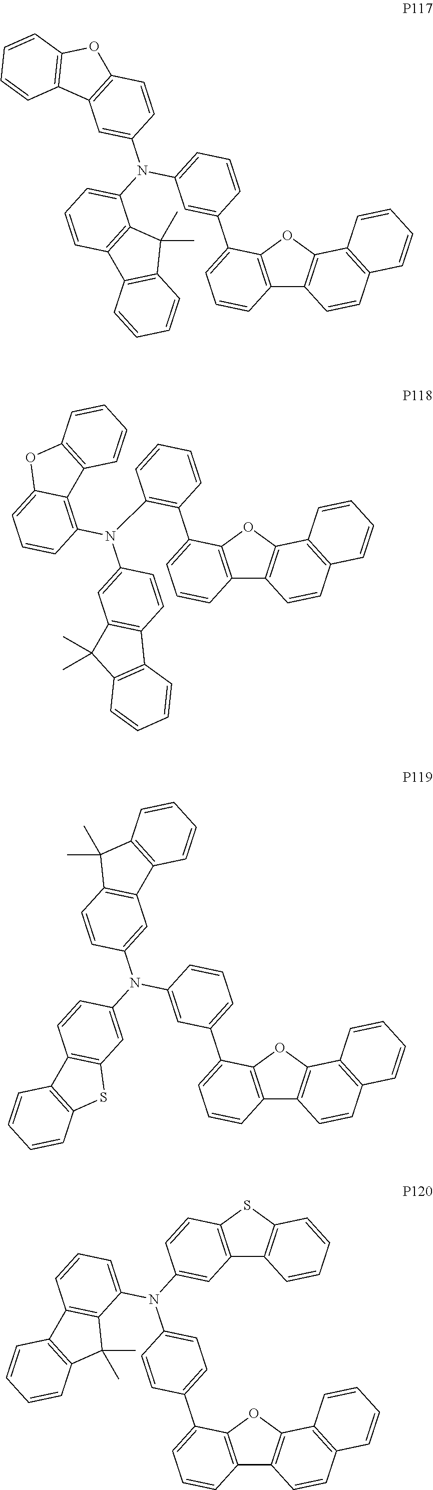 [SMILES: CC1(C)c2ccccc2-c2cccc(N(c3cccc(-c4cccc5c4oc4c6ccccc6ccc54)c3)c3ccc4oc5ccccc5c4c3)c21 ; CC1(C)c2ccccc2-c2ccc(N(c3ccccc3-c3cccc4c3oc3c5ccccc5ccc43)c3cccc4oc5ccccc5c34)cc21 ; CC1(C)c2ccccc2-c2cc(N(c3cccc(-c4cccc5c4oc4c6ccccc6ccc54)c3)c3ccc4c(c3)sc3ccccc34)ccc21 ; CC1(C)c2ccccc2-c2cccc(N(c3ccc(-c4cccc5c4oc4c6ccccc6ccc54)cc3)c3ccc4sc5ccccc5c4c3)c21]

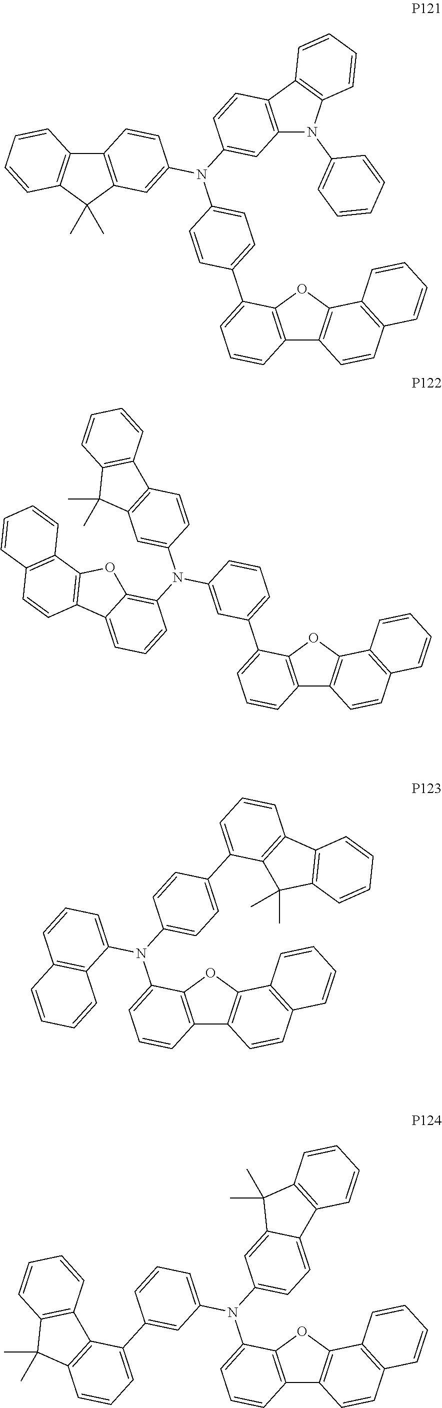

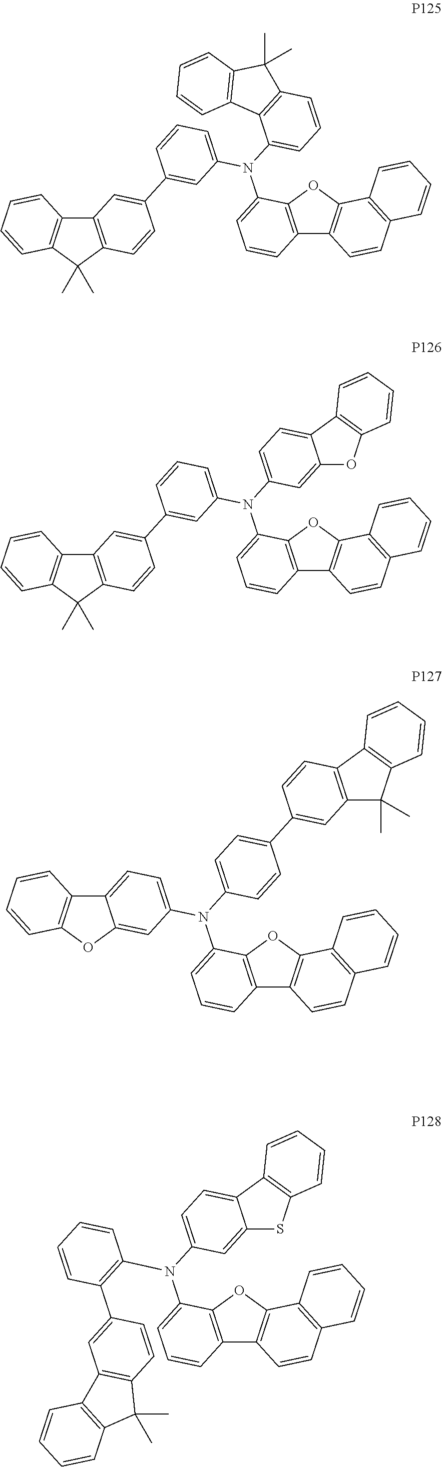 [SMILES: CC1(C)c2ccccc2-c2cc(-c3cccc(N(c4cccc5c4-c4ccccc4C5(C)C)c4cccc5c4oc4c6ccccc6ccc54)c3)ccc21 ; CC1(C)c2ccccc2-c2cc(-c3cccc(N(c4ccc5c(c4)oc4ccccc45)c4cccc5c4oc4c6ccccc6ccc54)c3)ccc21 ; CC1(C)c2ccccc2-c2ccc(-c3ccc(N(c4ccc5c(c4)oc4ccccc45)c4cccc5c4oc4c6ccccc6ccc54)cc3)cc21 ; CC1(C)c2ccccc2-c2cc(-c3ccccc3N(c3ccc4c(c3)sc3ccccc34)c3cccc4c3oc3c5ccccc5ccc43)ccc21]

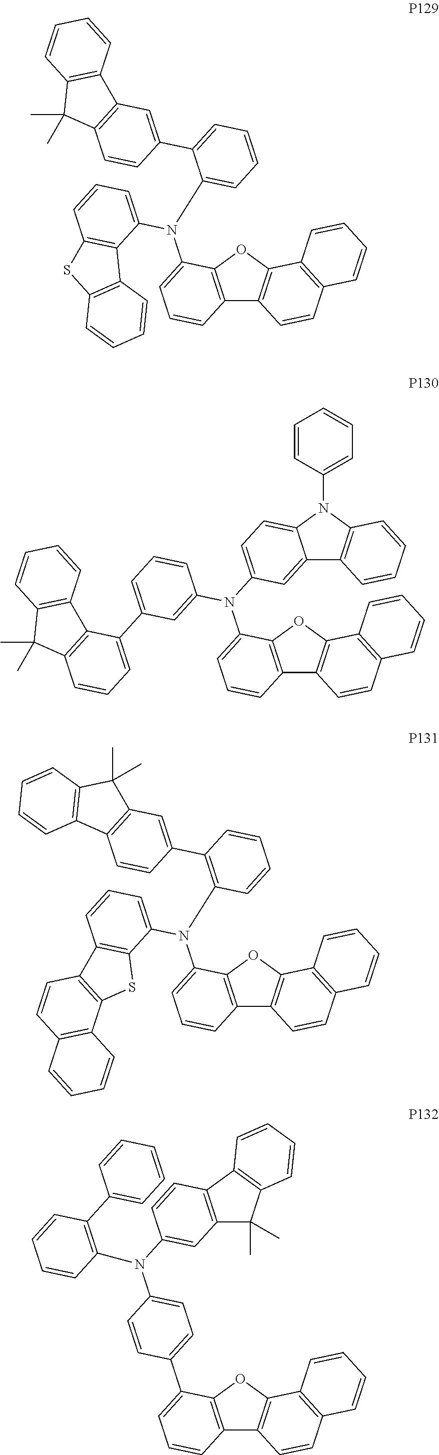

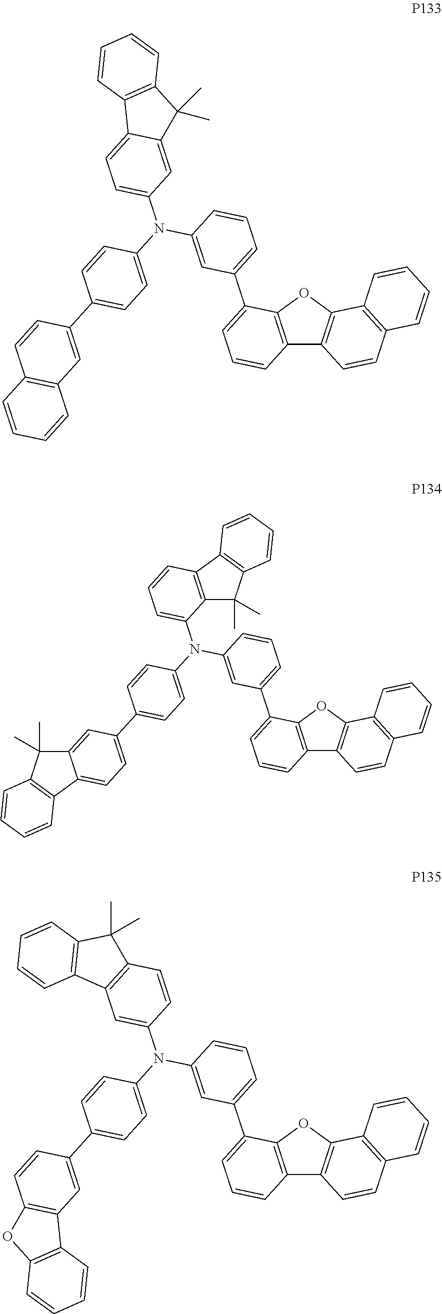 [SMILES: CC1(C)c2ccccc2-c2ccc(N(c3ccc(-c4ccc5ccccc5c4)cc3)c3cccc(-c4cccc5c4oc4c6ccccc6ccc54)c3)cc21 ; CC1(C)c2ccccc2-c2ccc(-c3ccc(N(c4cccc(-c5cccc6c5oc5c7ccccc7ccc65)c4)c4cccc5c4C(C)(C)c4ccccc4-5)cc3)cc21 ; CC1(C)c2ccccc2-c2cc(N(c3ccc(-c4ccc5oc6ccccc6c5c4)cc3)c3cccc(-c4cccc5c4oc4c6ccccc6ccc54)c3)ccc21]

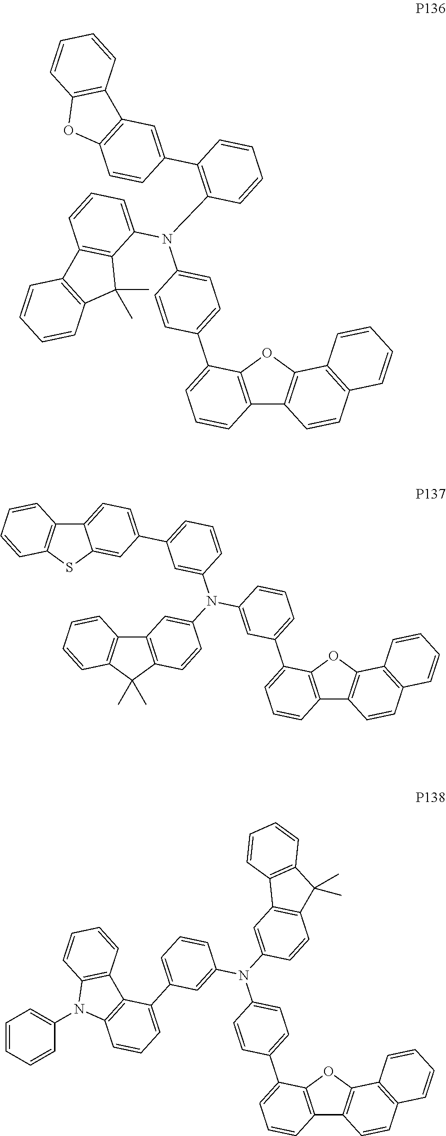 [SMILES: CC1(C)c2ccccc2-c2cccc(N(c3ccc(-c4cccc5c4oc4c6ccccc6ccc54)cc3)c3ccccc3-c3ccc4oc5ccccc5c4c3)c21 ; CC1(C)c2ccccc2-c2cc(N(c3cccc(-c4ccc5c(c4)sc4ccccc45)c3)c3cccc(-c4cccc5c4oc4c6ccccc6ccc54)c3)ccc21 ; CC1(C)c2ccccc2-c2cc(N(c3ccc(-c4cccc5c4oc4c6ccccc6ccc54)cc3)c3cccc(-c4cccc5c4c4ccccc4n5-c4ccccc4)c3)ccc21]

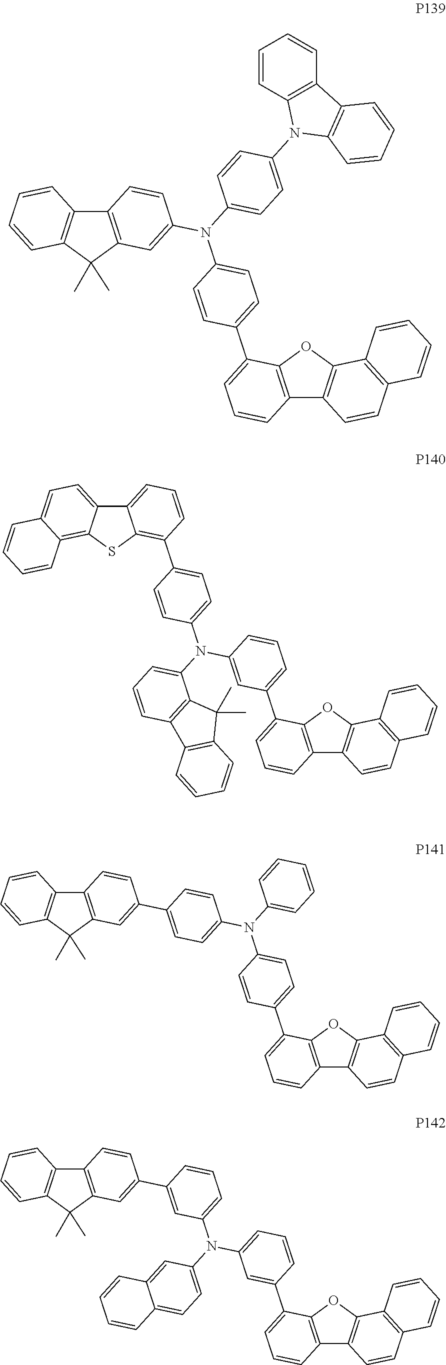 [SMILES: CC1(C)c2ccccc2-c2ccc(N(c3ccc(-c4cccc5c4oc4c6ccccc6ccc54)cc3)c3ccc(-n4c5ccccc5c5ccccc54)cc3)cc21 ; CC1(C)c2ccccc2-c2cccc(N(c3ccc(-c4cccc5c4sc4c6ccccc6ccc54)cc3)c3cccc(-c4cccc5c4oc4c6ccccc6ccc54)c3)c21 ; CC1(C)c2ccccc2-c2ccc(-c3ccc(N(c4ccccc4)c4ccc(-c5cccc6c5oc5c7ccccc7ccc65)cc4)cc3)cc21 ; CC1(C)c2ccccc2-c2ccc(-c3cccc(N(c4cccc(-c5cccc6c5oc5c7ccccc7ccc65)c4)c4ccc5ccccc5c4)c3)cc21]

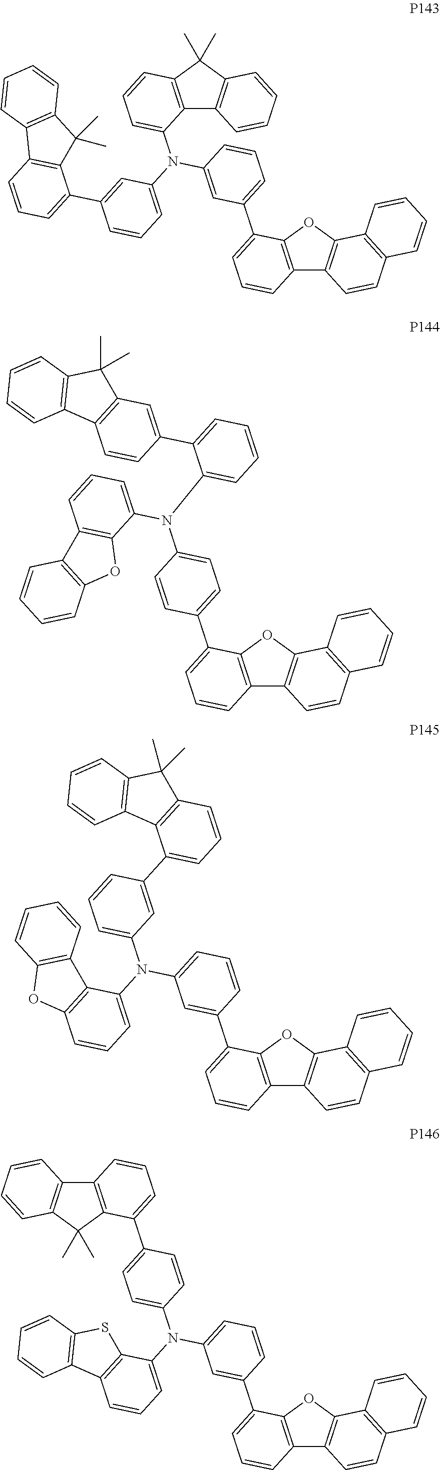 [SMILES: CC1(C)c2ccccc2-c2c(N(c3cccc(-c4cccc5c4C(C)(C)c4ccccc4-5)c3)c3cccc(-c4cccc5c4oc4c6ccccc6ccc54)c3)cccc21 ; CC1(C)c2ccccc2-c2ccc(-c3ccccc3N(c3ccc(-c4cccc5c4oc4c6ccccc6ccc54)cc3)c3cccc4c3oc3ccccc34)cc21 ; CC1(C)c2ccccc2-c2c(-c3cccc(N(c4cccc(-c5cccc6c5oc5c7ccccc7ccc65)c4)c4cccc5oc6ccccc6c45)c3)cccc21 ; CC1(C)c2ccccc2-c2cccc(-c3ccc(N(c4cccc(-c5cccc6c5oc5c7ccccc7ccc65)c4)c4cccc5c4sc4ccccc45)cc3)c21]

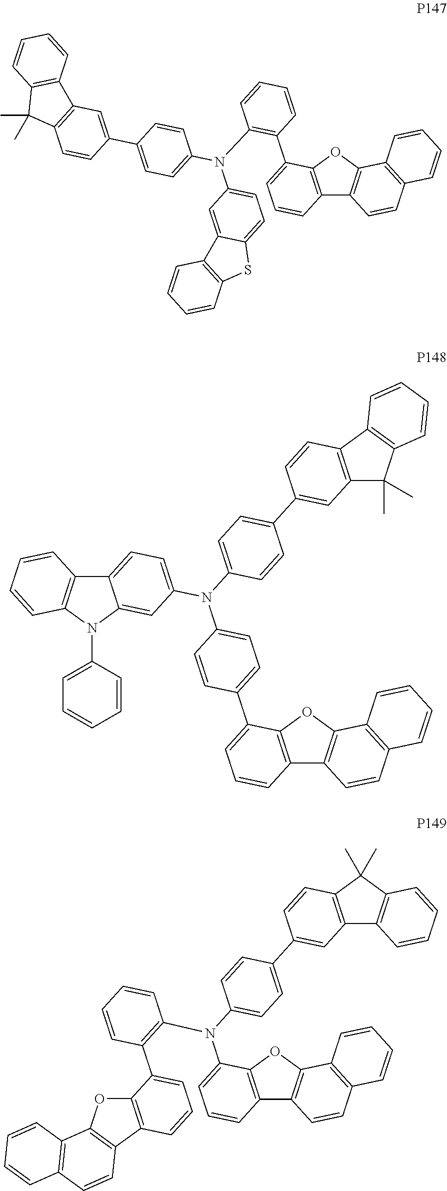 [SMILES: CC1(C)c2ccccc2-c2cc(-c3ccc(N(c4ccc5sc6ccccc6c5c4)c4ccccc4-c4cccc5c4oc4c6ccccc6ccc54)cc3)ccc21 ; CC1(C)c2ccccc2-c2ccc(-c3ccc(N(c4ccc(-c5cccc6c5oc5c7ccccc7ccc65)cc4)c4ccc5c6ccccc6n(-c6ccccc6)c5c4)cc3)cc21 ; CC1(C)c2ccccc2-c2cc(-c3ccc(N(c4ccccc4-c4cccc5c4oc4c6ccccc6ccc54)c4cccc5c4oc4c6ccccc6ccc54)cc3)ccc21]

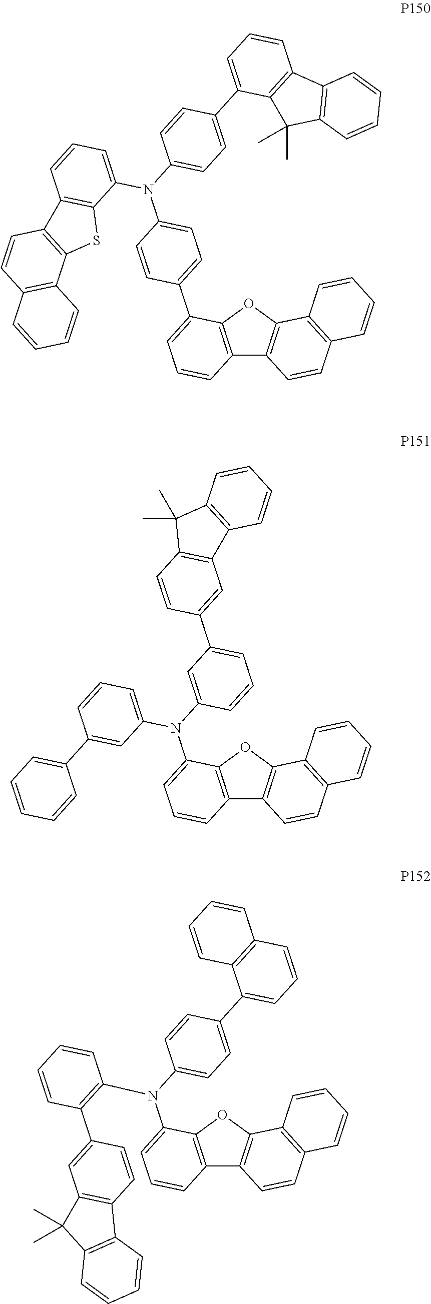 [SMILES: CC1(C)c2ccccc2-c2cccc(-c3ccc(N(c4ccc(-c5cccc6c5oc5c7ccccc7ccc65)cc4)c4cccc5c4sc4c6ccccc6ccc54)cc3)c21 ; CC1(C)c2ccccc2-c2cc(-c3cccc(N(c4cccc(-c5ccccc5)c4)c4cccc5c4oc4c6ccccc6ccc54)c3)ccc21 ; CC1(C)c2ccccc2-c2ccc(-c3ccccc3N(c3ccc(-c4cccc5ccccc45)cc3)c3cccc4c3oc3c5ccccc5ccc43)cc21]

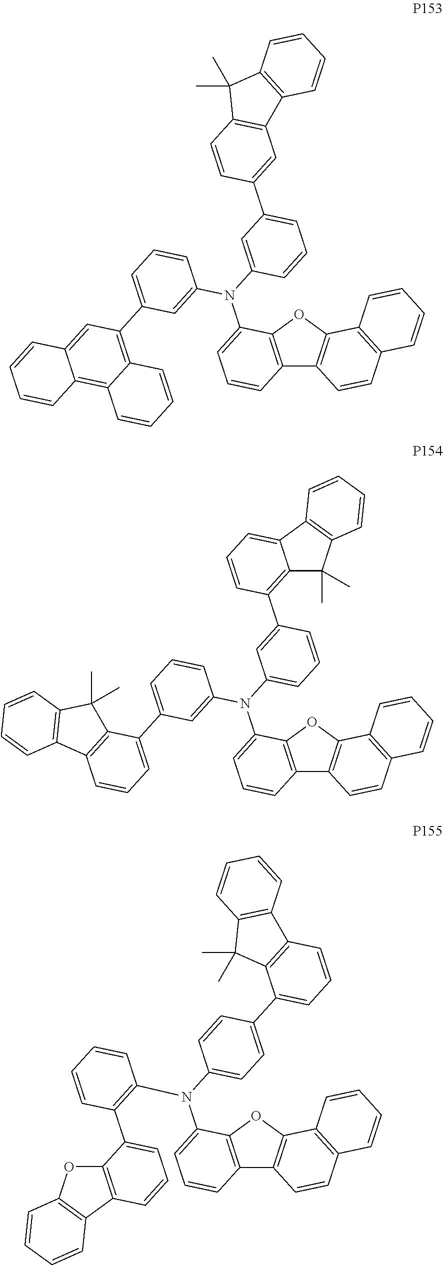 [SMILES: CC1(C)c2ccccc2-c2cc(-c3cccc(N(c4cccc(-c5cc6ccccc6c6ccccc56)c4)c4cccc5c4oc4c6ccccc6ccc54)c3)ccc21 ; CC1(C)c2ccccc2-c2cccc(-c3cccc(N(c4cccc(-c5cccc6c5C(C)(C)c5ccccc5-6)c4)c4cccc5c4oc4c6ccccc6ccc54)c3)c21 ; CC1(C)c2ccccc2-c2cccc(-c3ccc(N(c4ccccc4-c4cccc5c4oc4ccccc45)c4cccc5c4oc4c6ccccc6ccc54)cc3)c21]

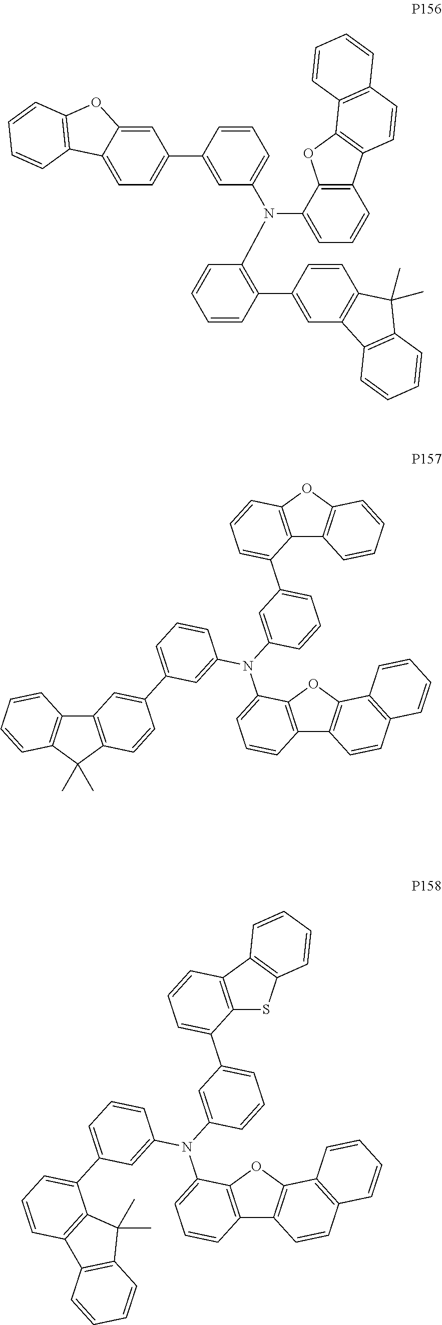 [SMILES: CC1(C)c2ccccc2-c2cc(-c3ccccc3N(c3cccc(-c4ccc5c(c4)oc4ccccc45)c3)c3cccc4c3oc3c5ccccc5ccc43)ccc21 ; CC1(C)c2ccccc2-c2cc(-c3cccc(N(c4cccc(-c5cccc6oc7ccccc7c56)c4)c4cccc5c4oc4c6ccccc6ccc54)c3)ccc21 ; CC1(C)c2ccccc2-c2cccc(-c3cccc(N(c4cccc(-c5cccc6c5sc5ccccc56)c4)c4cccc5c4oc4c6ccccc6ccc54)c3)c21]

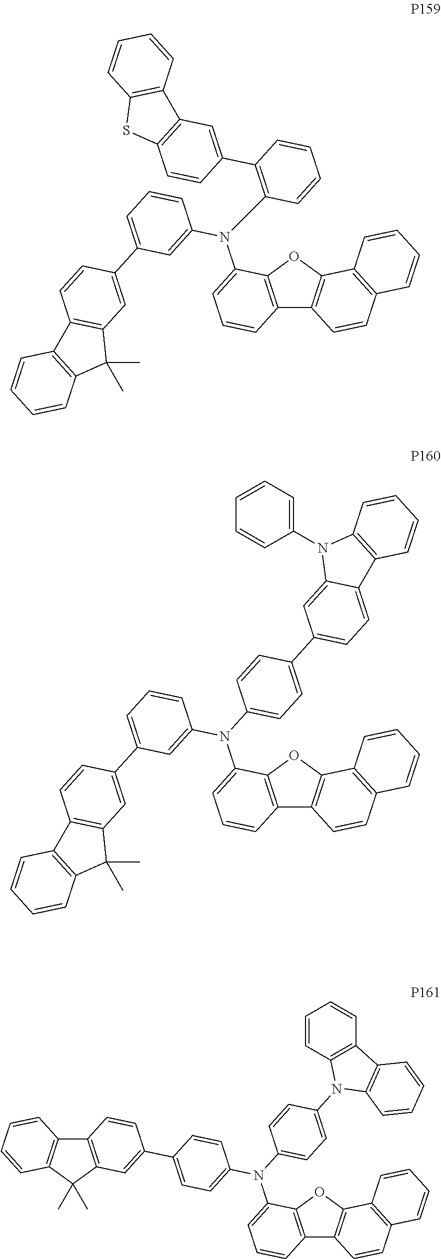 [SMILES: CC1(C)c2ccccc2-c2ccc(-c3cccc(N(c4ccccc4-c4ccc5sc6ccccc6c5c4)c4cccc5c4oc4c6ccccc6ccc54)c3)cc21 ; CC1(C)c2ccccc2-c2ccc(-c3cccc(N(c4ccc(-c5ccc6c7ccccc7n(-c7ccccc7)c6c5)cc4)c4cccc5c4oc4c6ccccc6ccc54)c3)cc21 ; CC1(C)c2ccccc2-c2ccc(-c3ccc(N(c4ccc(-n5c6ccccc6c6ccccc65)cc4)c4cccc5c4oc4c6ccccc6ccc54)cc3)cc21]

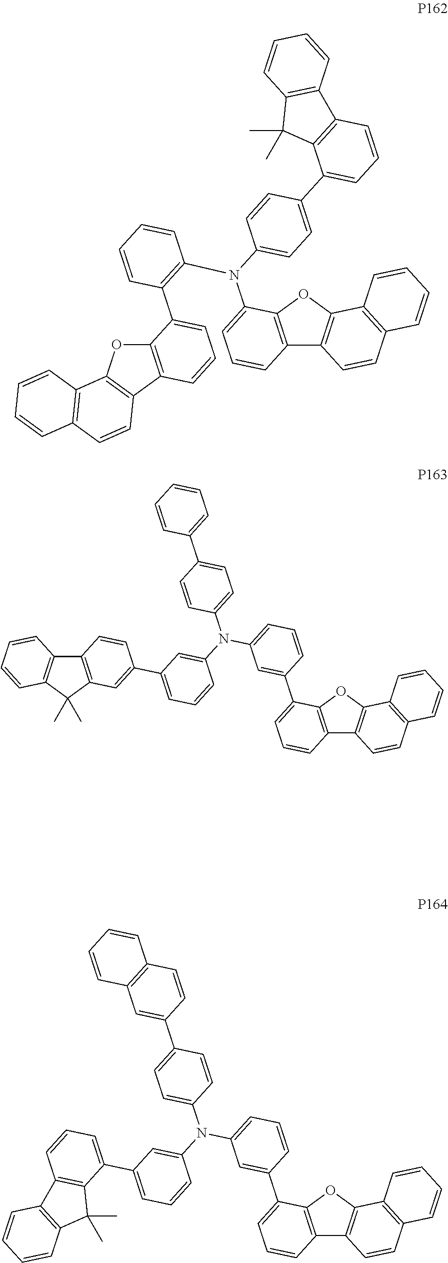 [SMILES: CC1(C)c2ccccc2-c2cccc(-c3ccc(N(c4ccccc4-c4cccc5c4oc4c6ccccc6ccc54)c4cccc5c4oc4c6ccccc6ccc54)cc3)c21 ; CC1(C)c2ccccc2-c2ccc(-c3cccc(N(c4ccc(-c5ccccc5)cc4)c4cccc(-c5cccc6c5oc5c7ccccc7ccc65)c4)c3)cc21 ; CC1(C)c2ccccc2-c2cccc(-c3cccc(N(c4ccc(-c5ccc6ccccc6c5)cc4)c4cccc(-c5cccc6c5oc5c7ccccc7ccc65)c4)c3)c21]

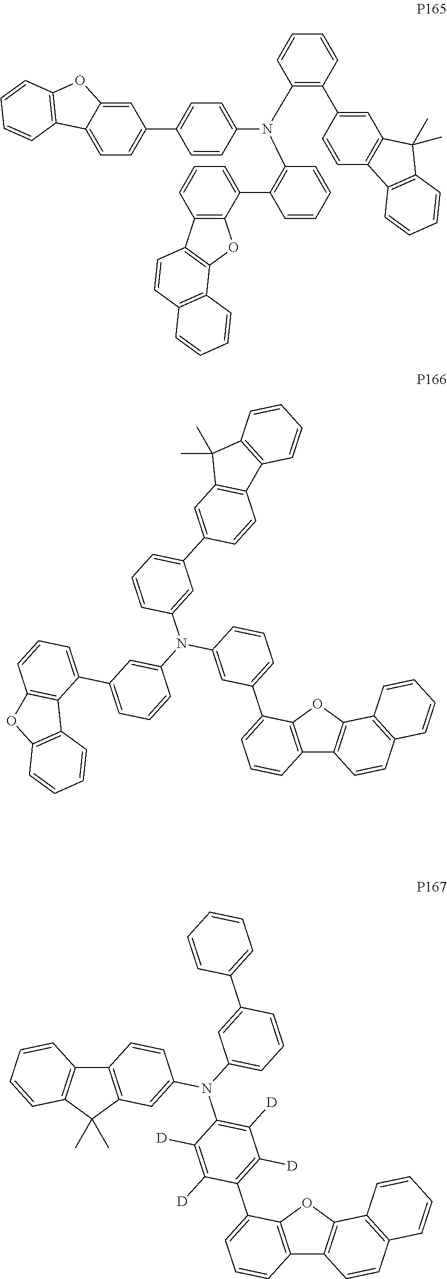 [SMILES: CC1(C)c2ccccc2-c2ccc(-c3ccccc3N(c3ccc(-c4ccc5c(c4)oc4ccccc45)cc3)c3ccccc3-c3cccc4c3oc3c5ccccc5ccc43)cc21 ; CC1(C)c2ccccc2-c2ccc(-c3cccc(N(c4cccc(-c5cccc6c5oc5c7ccccc7ccc65)c4)c4cccc(-c5cccc6oc7ccccc7c56)c4)c3)cc21 ; [2H]c1c([2H])c(N(c2cccc(-c3ccccc3)c2)c2ccc3c(c2)C(C)(C)c2ccccc2-3)c([2H])c([2H])c1-c1cccc2c1oc1c3ccccc3ccc21]

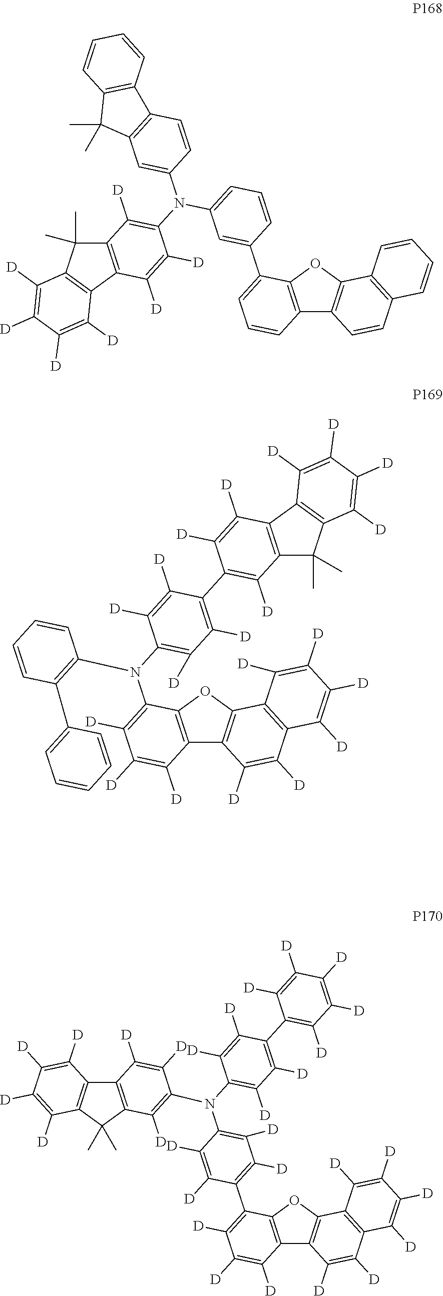

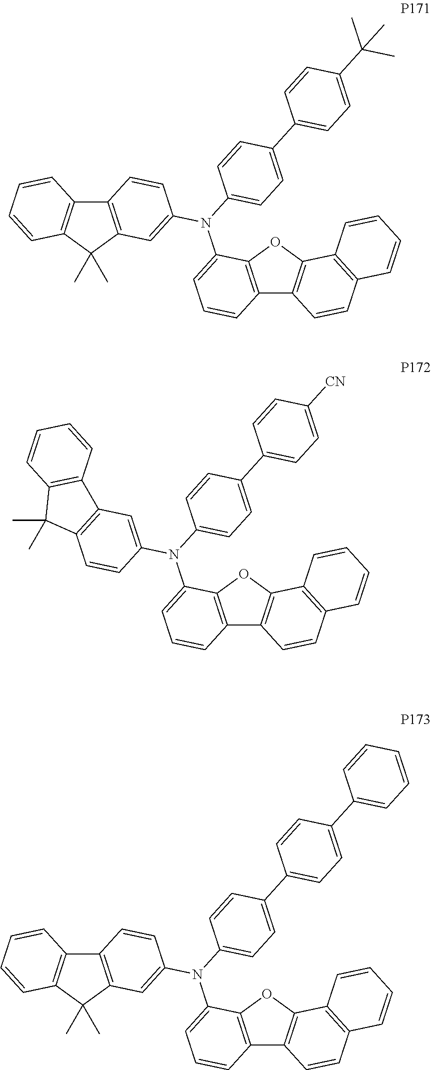 [SMILES: CC(C)(C)c1ccc(-c2ccc(N(c3ccc4c(c3)C(C)(C)c3ccccc3-4)c3cccc4c3oc3c5ccccc5ccc43)cc2)cc1 ; CC1(C)c2ccccc2-c2cc(N(c3ccc(-c4ccc(C#N)cc4)cc3)c3cccc4c3oc3c5ccccc5ccc43)ccc21 ; CC1(C)c2ccccc2-c2ccc(N(c3ccc(-c4ccc(-c5ccccc5)cc4)cc3)c3cccc4c3oc3c5ccccc5ccc43)cc21]

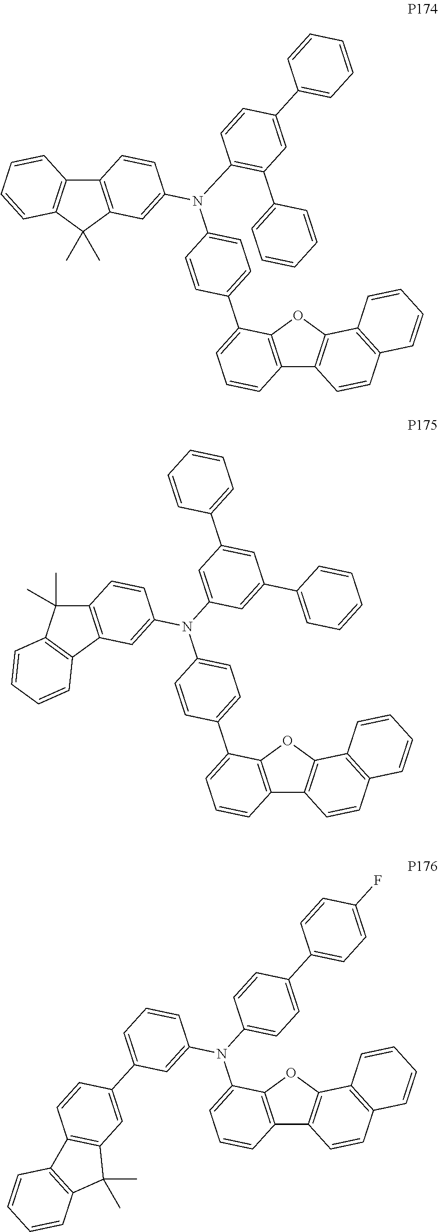 [SMILES: CC1(C)c2ccccc2-c2ccc(N(c3ccc(-c4cccc5c4oc4c6ccccc6ccc54)cc3)c3ccc(-c4ccccc4)cc3-c3ccccc3)cc21 ; CC1(C)c2ccccc2-c2cc(N(c3ccc(-c4cccc5c4oc4c6ccccc6ccc54)cc3)c3cc(-c4ccccc4)cc(-c4ccccc4)c3)ccc21 ; CC1(C)c2ccccc2-c2ccc(-c3cccc(N(c4ccc(-c5ccc(F)cc5)cc4)c4cccc5c4oc4c6ccccc6ccc54)c3)cc21]

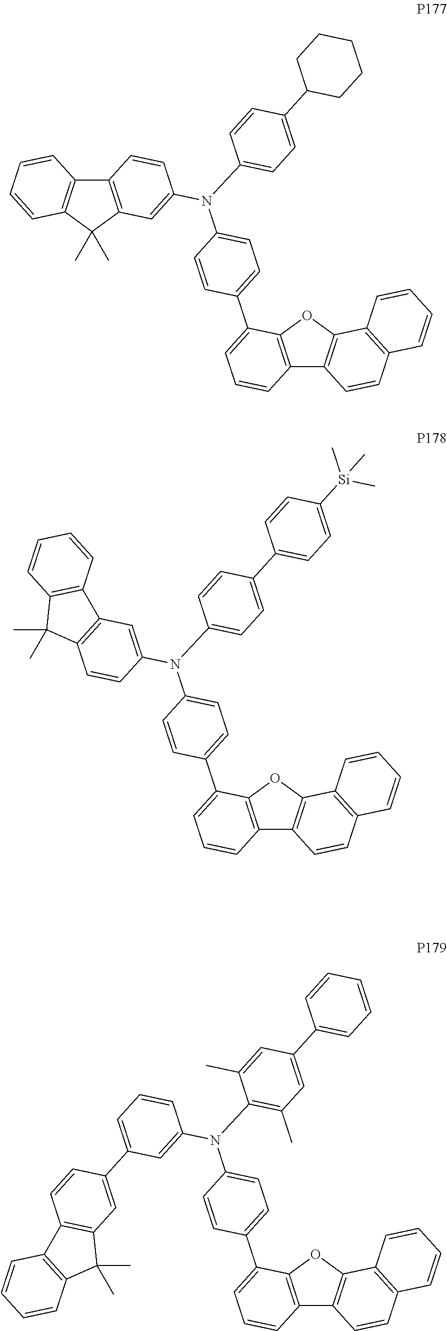 [SMILES: CC1(C)c2ccccc2-c2ccc(N(c3ccc(-c4cccc5c4oc4c6ccccc6ccc54)cc3)c3ccc(C4CCCCC4)cc3)cc21 ; CC1(C)c2ccccc2-c2cc(N(c3ccc(-c4ccc([Si](C)(C)C)cc4)cc3)c3ccc(-c4cccc5c4oc4c6ccccc6ccc54)cc3)ccc21 ; Cc1cc(-c2ccccc2)cc(C)c1N(c1ccc(-c2cccc3c2oc2c4ccccc4ccc32)cc1)c1cccc(-c2ccc3c(c2)C(C)(C)c2ccccc2-3)c1]

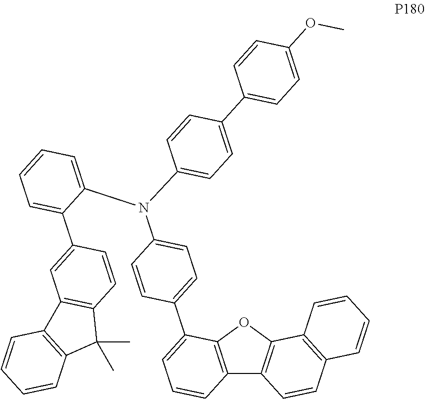 [SMILES: COc1ccc(-c2ccc(N(c3ccc(-c4cccc5c4oc4c6ccccc6ccc54)cc3)c3ccccc3-c3ccc4c(c3)-c3ccccc3C4(C)C)cc2)cc1]

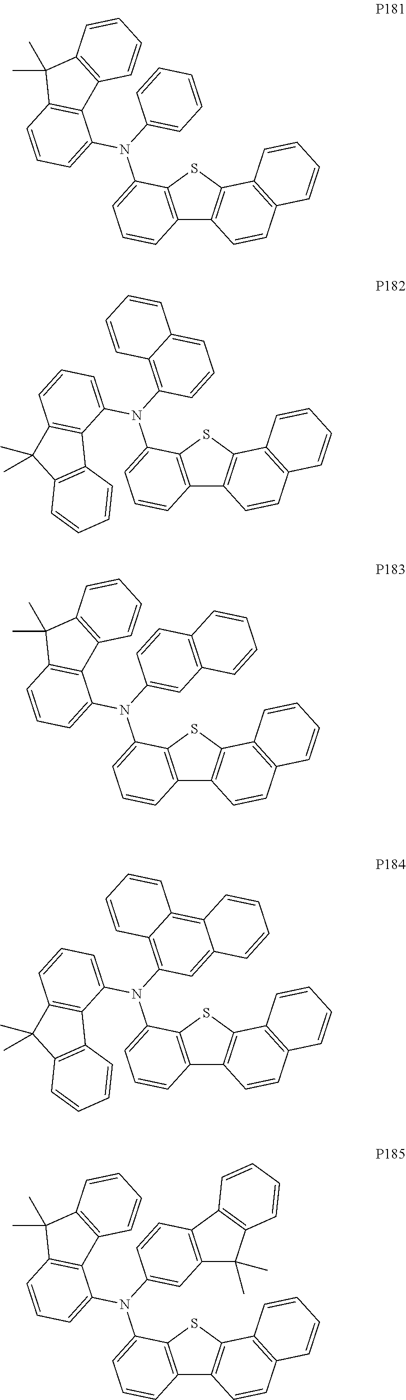 [SMILES: CC1(C)c2ccccc2-c2c(N(c3ccccc3)c3cccc4c3sc3c5ccccc5ccc43)cccc21 ; CC1(C)c2ccccc2-c2c(N(c3cccc4ccccc34)c3cccc4c3sc3c5ccccc5ccc43)cccc21 ; CC1(C)c2ccccc2-c2c(N(c3ccc4ccccc4c3)c3cccc4c3sc3c5ccccc5ccc43)cccc21 ; CC1(C)c2ccccc2-c2c(N(c3cc4ccccc4c4ccccc34)c3cccc4c3sc3c5ccccc5ccc43)cccc21 ; CC1(C)c2ccccc2-c2ccc(N(c3cccc4c3-c3ccccc3C4(C)C)c3cccc4c3sc3c5ccccc5ccc43)cc21]

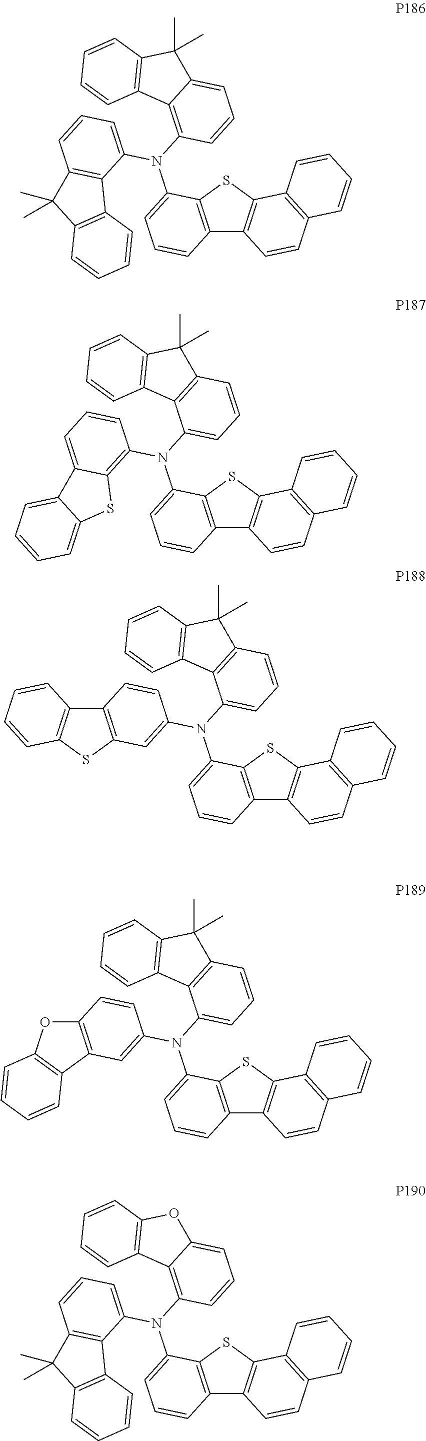

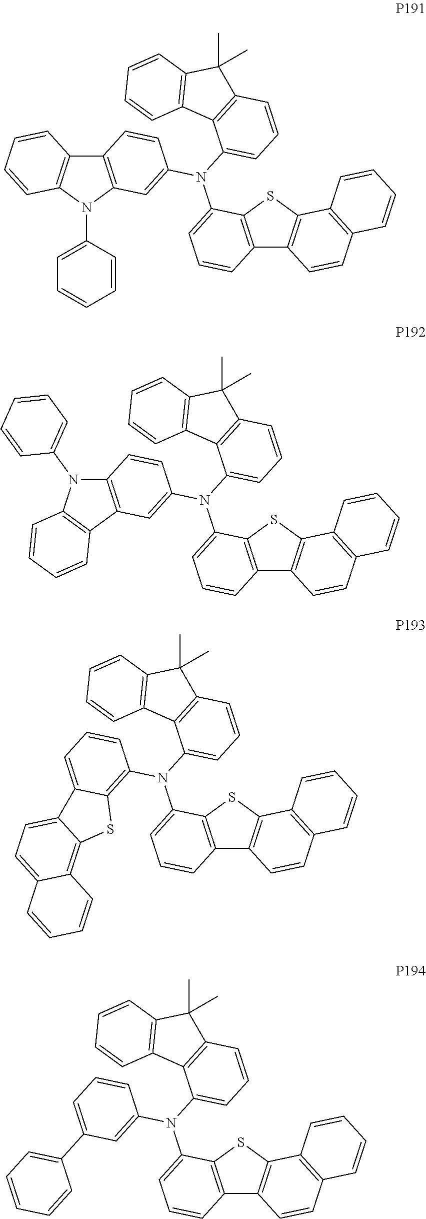 [SMILES: CC1(C)c2ccccc2-c2c(N(c3ccc4c5ccccc5n(-c5ccccc5)c4c3)c3cccc4c3sc3c5ccccc5ccc43)cccc21 ; CC1(C)c2ccccc2-c2c(N(c3ccc4c(c3)c3ccccc3n4-c3ccccc3)c3cccc4c3sc3c5ccccc5ccc43)cccc21 ; CC1(C)c2ccccc2-c2c(N(c3cccc4c3sc3c5ccccc5ccc43)c3cccc4c3sc3c5ccccc5ccc43)cccc21 ; CC1(C)c2ccccc2-c2c(N(c3cccc(-c4ccccc4)c3)c3cccc4c3sc3c5ccccc5ccc43)cccc21]

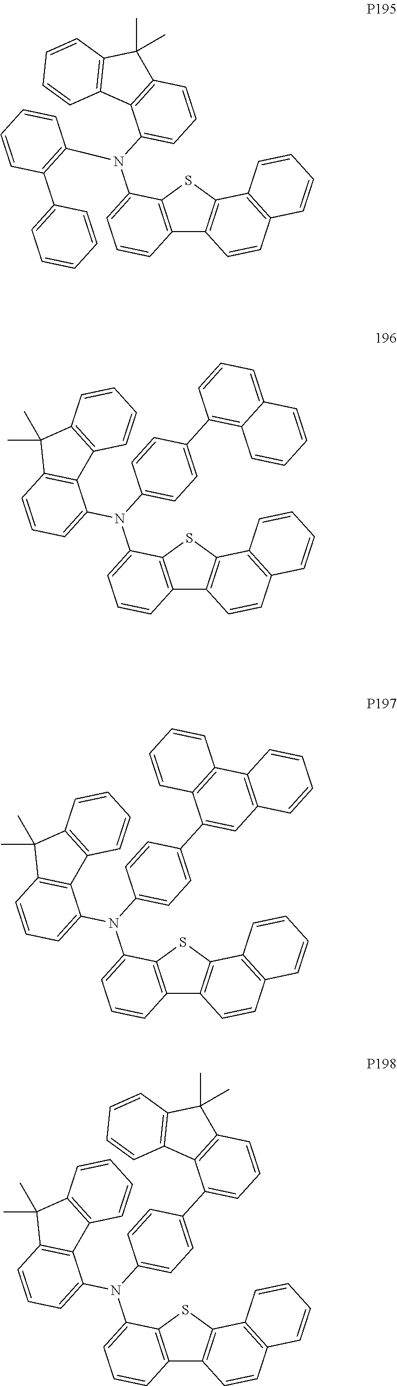 [SMILES: CC1(C)c2ccccc2-c2c(N(c3ccccc3-c3ccccc3)c3cccc4c3sc3c5ccccc5ccc43)cccc21 ; CC1(C)c2ccccc2-c2c(N(c3ccc(-c4cccc5ccccc45)cc3)c3cccc4c3sc3c5ccccc5ccc43)cccc21 ; CC1(C)c2ccccc2-c2c(N(c3ccc(-c4cc5ccccc5c5ccccc45)cc3)c3cccc4c3sc3c5ccccc5ccc43)cccc21 ; CC1(C)c2ccccc2-c2c(-c3ccc(N(c4cccc5c4-c4ccccc4C5(C)C)c4cccc5c4sc4c6ccccc6ccc54)cc3)cccc21]

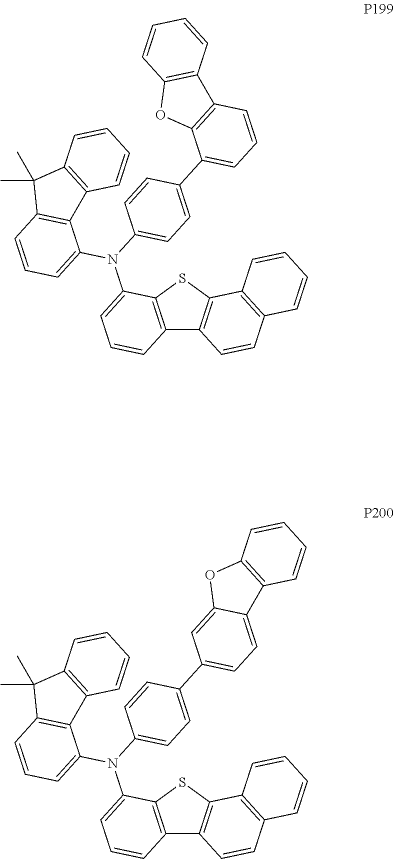 [SMILES: CC1(C)c2ccccc2-c2c(N(c3ccc(-c4cccc5c4oc4ccccc45)cc3)c3cccc4c3sc3c5ccccc5ccc43)cccc21 ; CC1(C)c2ccccc2-c2c(N(c3ccc(-c4ccc5c(c4)oc4ccccc45)cc3)c3cccc4c3sc3c5ccccc5ccc43)cccc21]

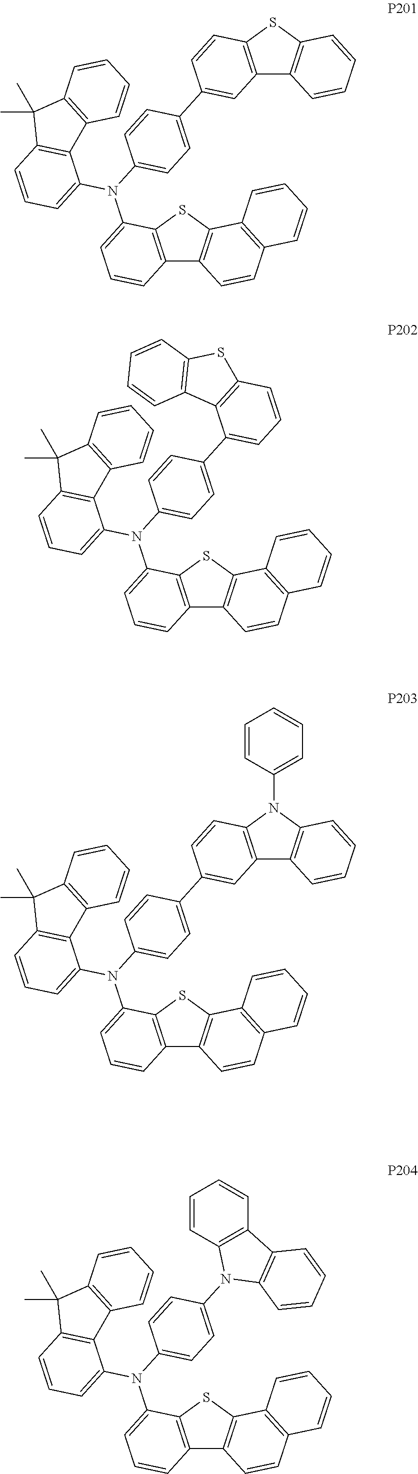 [SMILES: CC1(C)c2ccccc2-c2c(N(c3ccc(-c4ccc5sc6ccccc6c5c4)cc3)c3cccc4c3sc3c5ccccc5ccc43)cccc21 ; CC1(C)c2ccccc2-c2c(N(c3ccc(-c4cccc5sc6ccccc6c45)cc3)c3cccc4c3sc3c5ccccc5ccc43)cccc21 ; CC1(C)c2ccccc2-c2c(N(c3ccc(-c4ccc5c(c4)c4ccccc4n5-c4ccccc4)cc3)c3cccc4c3sc3c5ccccc5ccc43)cccc21 ; CC1(C)c2ccccc2-c2c(N(c3ccc(-n4c5ccccc5c5ccccc54)cc3)c3cccc4c3sc3c5ccccc5ccc43)cccc21]

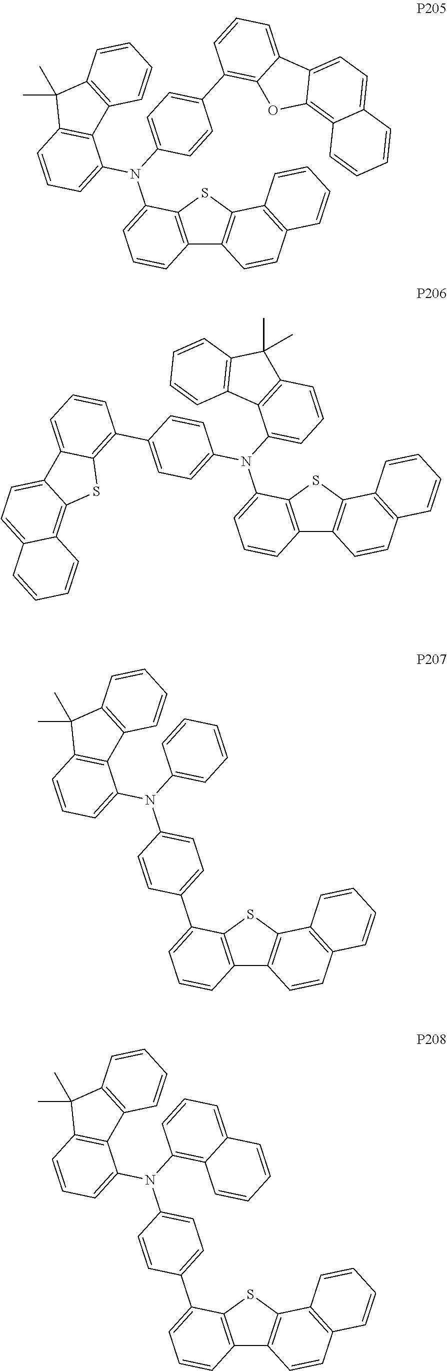 [SMILES: CC1(C)c2ccccc2-c2c(N(c3ccc(-c4cccc5c4oc4c6ccccc6ccc54)cc3)c3cccc4c3sc3c5ccccc5ccc43)cccc21 ; CC1(C)c2ccccc2-c2c(N(c3ccc(-c4cccc5c4sc4c6ccccc6ccc54)cc3)c3cccc4c3sc3c5ccccc5ccc43)cccc21 ; CC1(C)c2ccccc2-c2c(N(c3ccccc3)c3ccc(-c4cccc5c4sc4c6ccccc6ccc54)cc3)cccc21 ; CC1(C)c2ccccc2-c2c(N(c3ccc(-c4cccc5c4sc4c6ccccc6ccc54)cc3)c3cccc4ccccc34)cccc21]

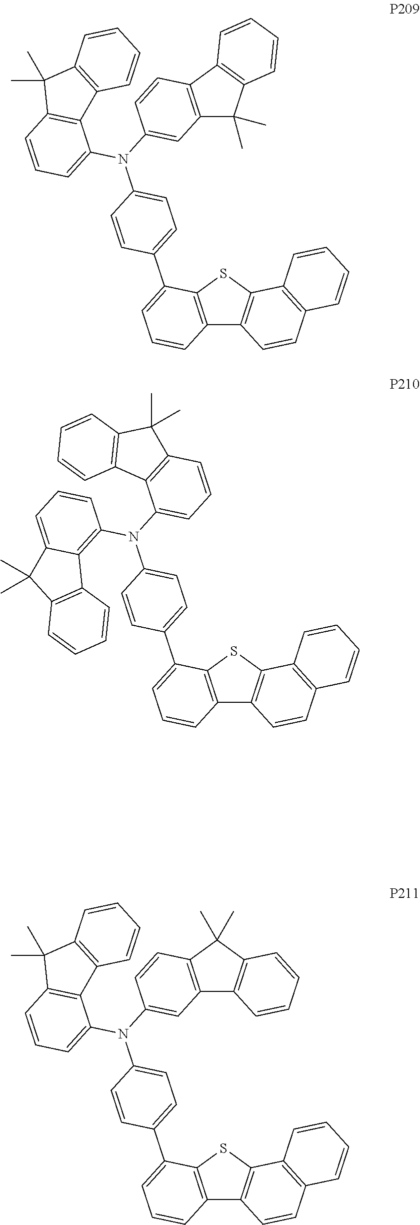 [SMILES: CC1(C)c2ccccc2-c2ccc(N(c3ccc(-c4cccc5c4sc4c6ccccc6ccc54)cc3)c3cccc4c3-c3ccccc3C4(C)C)cc21 ; CC1(C)c2ccccc2-c2c(N(c3ccc(-c4cccc5c4sc4c6ccccc6ccc54)cc3)c3cccc4c3-c3ccccc3C4(C)C)cccc21 ; CC1(C)c2ccccc2-c2cc(N(c3ccc(-c4cccc5c4sc4c6ccccc6ccc54)cc3)c3cccc4c3-c3ccccc3C4(C)C)ccc21]

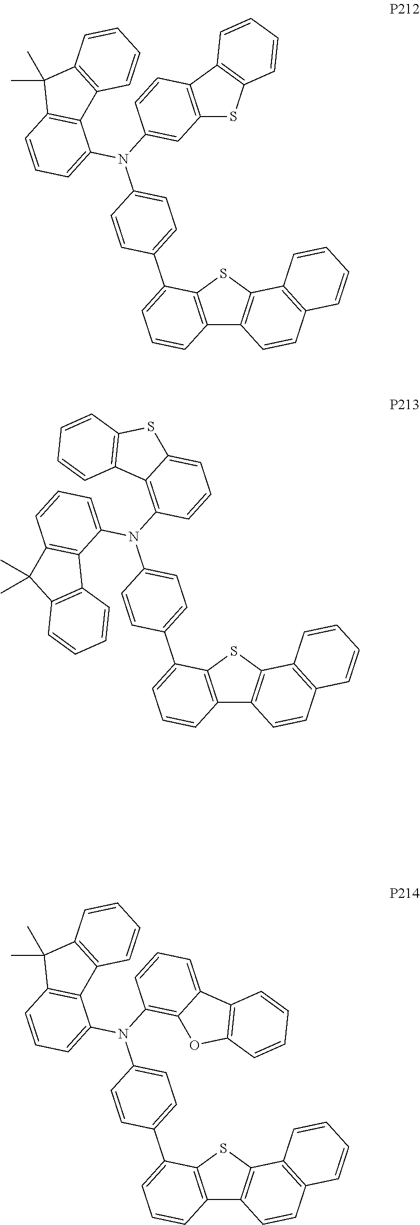 [SMILES: CC1(C)c2ccccc2-c2c(N(c3ccc(-c4cccc5c4sc4c6ccccc6ccc54)cc3)c3ccc4c(c3)sc3ccccc34)cccc21 ; CC1(C)c2ccccc2-c2c(N(c3ccc(-c4cccc5c4sc4c6ccccc6ccc54)cc3)c3cccc4sc5ccccc5c34)cccc21 ; CC1(C)c2ccccc2-c2c(N(c3ccc(-c4cccc5c4sc4c6ccccc6ccc54)cc3)c3cccc4c3oc3ccccc34)cccc21]

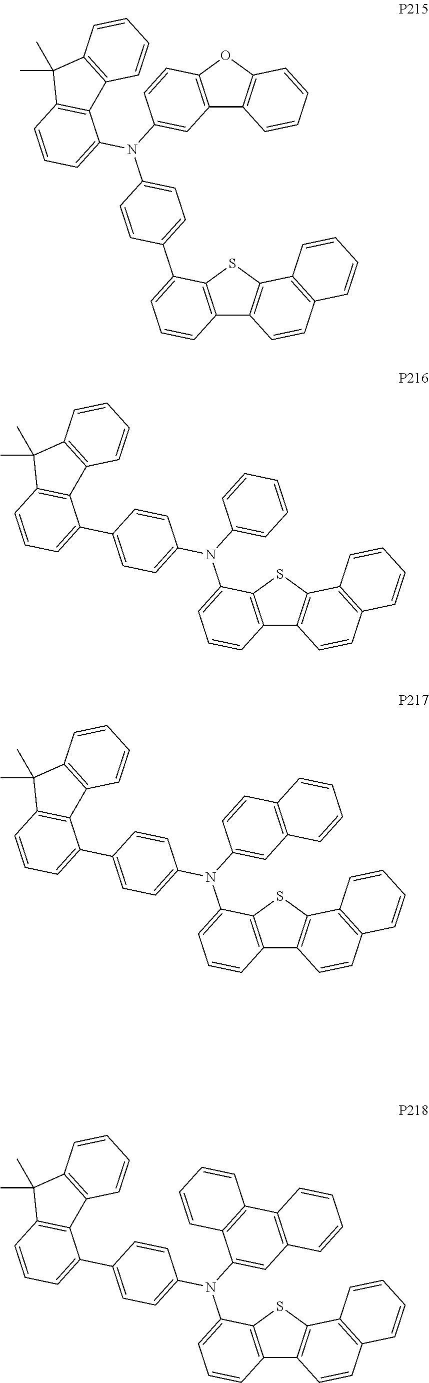 [SMILES: CC1(C)c2ccccc2-c2c(N(c3ccc(-c4cccc5c4sc4c6ccccc6ccc54)cc3)c3ccc4oc5ccccc5c4c3)cccc21 ; CC1(C)c2ccccc2-c2c(-c3ccc(N(c4ccccc4)c4cccc5c4sc4c6ccccc6ccc54)cc3)cccc21 ; CC1(C)c2ccccc2-c2c(-c3ccc(N(c4ccc5ccccc5c4)c4cccc5c4sc4c6ccccc6ccc54)cc3)cccc21 ; CC1(C)c2ccccc2-c2c(-c3ccc(N(c4cc5ccccc5c5ccccc45)c4cccc5c4sc4c6ccccc6ccc54)cc3)cccc21]

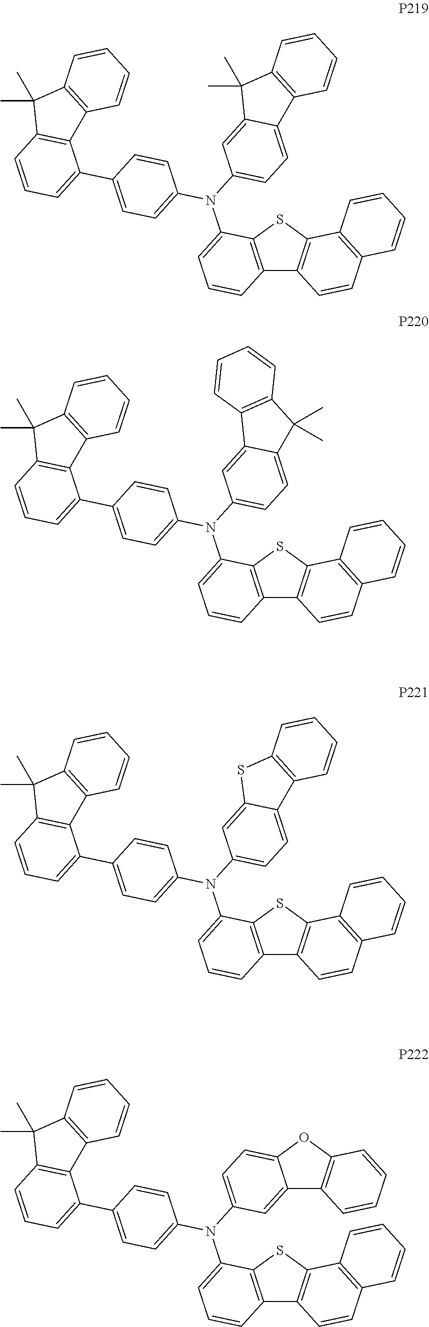 [SMILES: CC1(C)c2ccccc2-c2ccc(N(c3ccc(-c4cccc5c4-c4ccccc4C5(C)C)cc3)c3cccc4c3sc3c5ccccc5ccc43)cc21 ; CC1(C)c2ccccc2-c2cc(N(c3ccc(-c4cccc5c4-c4ccccc4C5(C)C)cc3)c3cccc4c3sc3c5ccccc5ccc43)ccc21 ; CC1(C)c2ccccc2-c2c(-c3ccc(N(c4ccc5c(c4)sc4ccccc45)c4cccc5c4sc4c6ccccc6ccc54)cc3)cccc21 ; CC1(C)c2ccccc2-c2c(-c3ccc(N(c4ccc5oc6ccccc6c5c4)c4cccc5c4sc4c6ccccc6ccc54)cc3)cccc21]

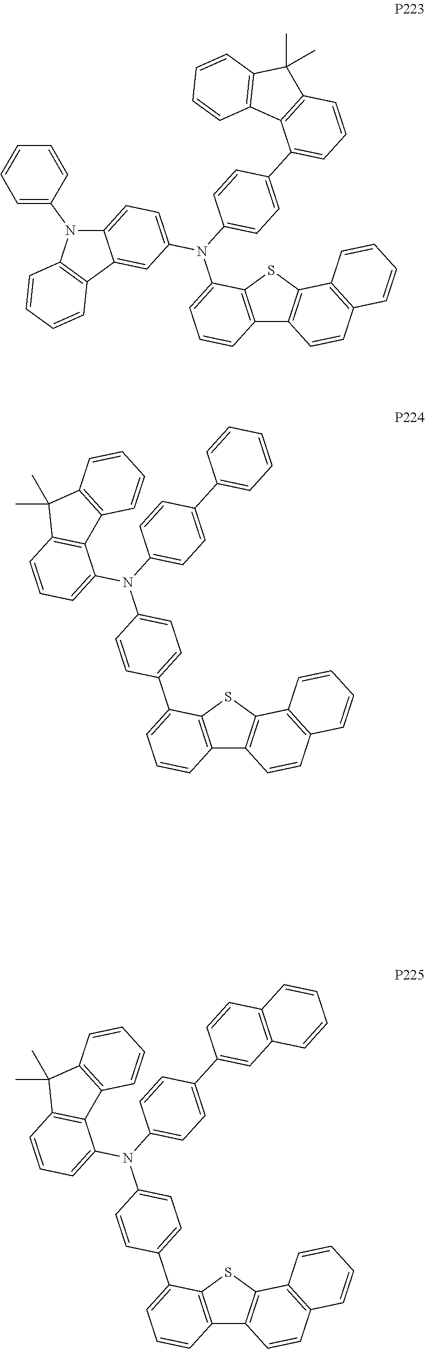 [SMILES: CC1(C)c2ccccc2-c2c(-c3ccc(N(c4ccc5c(c4)c4ccccc4n5-c4ccccc4)c4cccc5c4sc4c6ccccc6ccc54)cc3)cccc21 ; CC1(C)c2ccccc2-c2c(N(c3ccc(-c4ccccc4)cc3)c3ccc(-c4cccc5c4sc4c6ccccc6ccc54)cc3)cccc21 ; CC1(C)c2ccccc2-c2c(N(c3ccc(-c4ccc5ccccc5c4)cc3)c3ccc(-c4cccc5c4sc4c6ccccc6ccc54)cc3)cccc21]

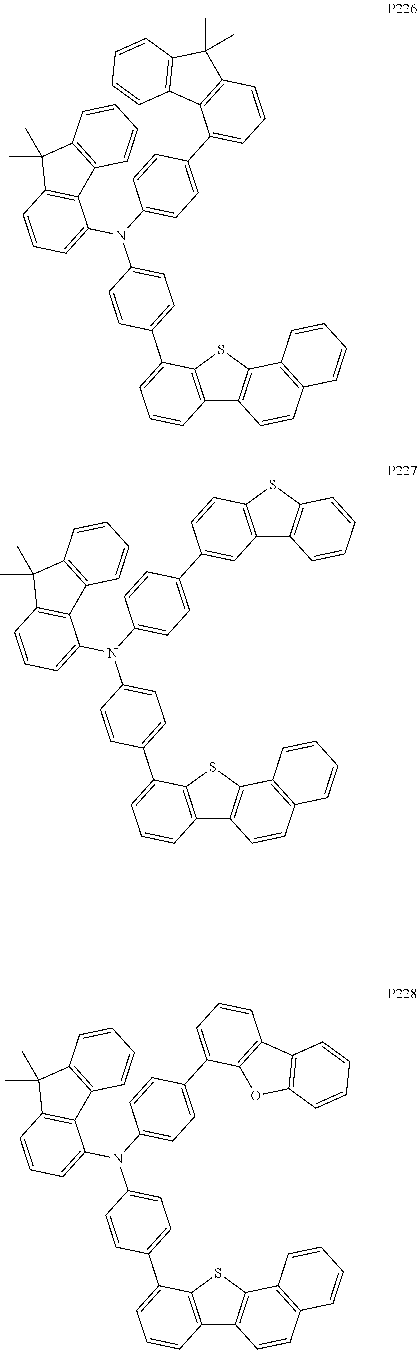 [SMILES: CC1(C)c2ccccc2-c2c(-c3ccc(N(c4ccc(-c5cccc6c5sc5c7ccccc7ccc65)cc4)c4cccc5c4-c4ccccc4C5(C)C)cc3)cccc21 ; CC1(C)c2ccccc2-c2c(N(c3ccc(-c4ccc5sc6ccccc6c5c4)cc3)c3ccc(-c4cccc5c4sc4c6ccccc6ccc54)cc3)cccc21 ; CC1(C)c2ccccc2-c2c(N(c3ccc(-c4cccc5c4oc4ccccc45)cc3)c3ccc(-c4cccc5c4sc4c6ccccc6ccc54)cc3)cccc21]

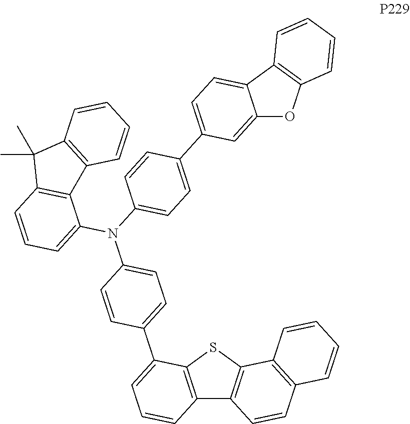 [SMILES: CC1(C)c2ccccc2-c2c(N(c3ccc(-c4ccc5c(c4)oc4ccccc45)cc3)c3ccc(-c4cccc5c4sc4c6ccccc6ccc54)cc3)cccc21]

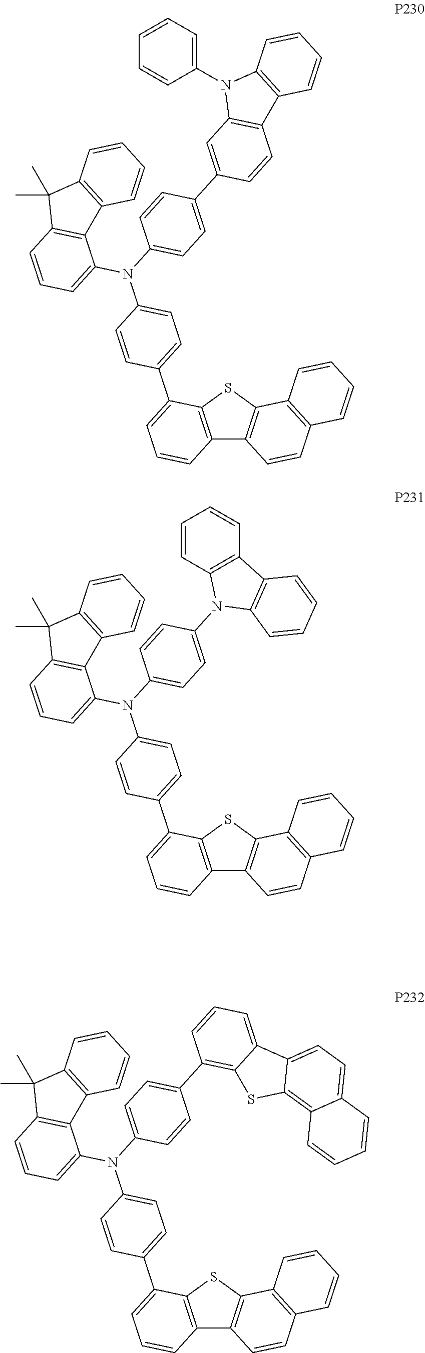 [SMILES: CC1(C)c2ccccc2-c2c(N(c3ccc(-c4ccc5c6ccccc6n(-c6ccccc6)c5c4)cc3)c3ccc(-c4cccc5c4sc4c6ccccc6ccc54)cc3)cccc21 ; CC1(C)c2ccccc2-c2c(N(c3ccc(-c4cccc5c4sc4c6ccccc6ccc54)cc3)c3ccc(-n4c5ccccc5c5ccccc54)cc3)cccc21 ; CC1(C)c2ccccc2-c2c(N(c3ccc(-c4cccc5c4sc4c6ccccc6ccc54)cc3)c3ccc(-c4cccc5c4sc4c6ccccc6ccc54)cc3)cccc21]

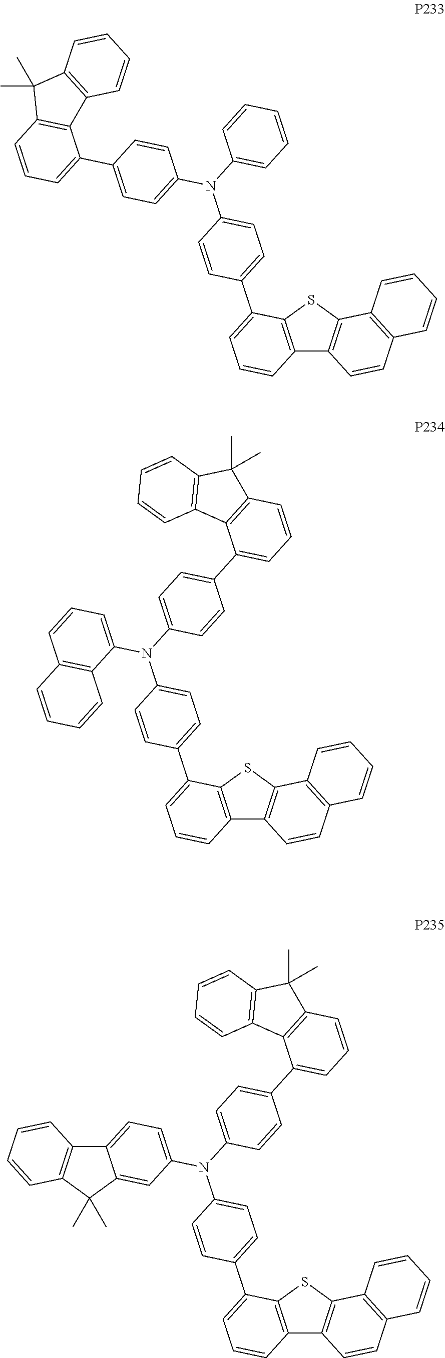 [SMILES: CC1(C)c2ccccc2-c2c(-c3ccc(N(c4ccccc4)c4ccc(-c5cccc6c5sc5c7ccccc7ccc65)cc4)cc3)cccc21 ; CC1(C)c2ccccc2-c2c(-c3ccc(N(c4ccc(-c5cccc6c5sc5c7ccccc7ccc65)cc4)c4cccc5ccccc45)cc3)cccc21 ; CC1(C)c2ccccc2-c2ccc(N(c3ccc(-c4cccc5c4-c4ccccc4C5(C)C)cc3)c3ccc(-c4cccc5c4sc4c6ccccc6ccc54)cc3)cc21]

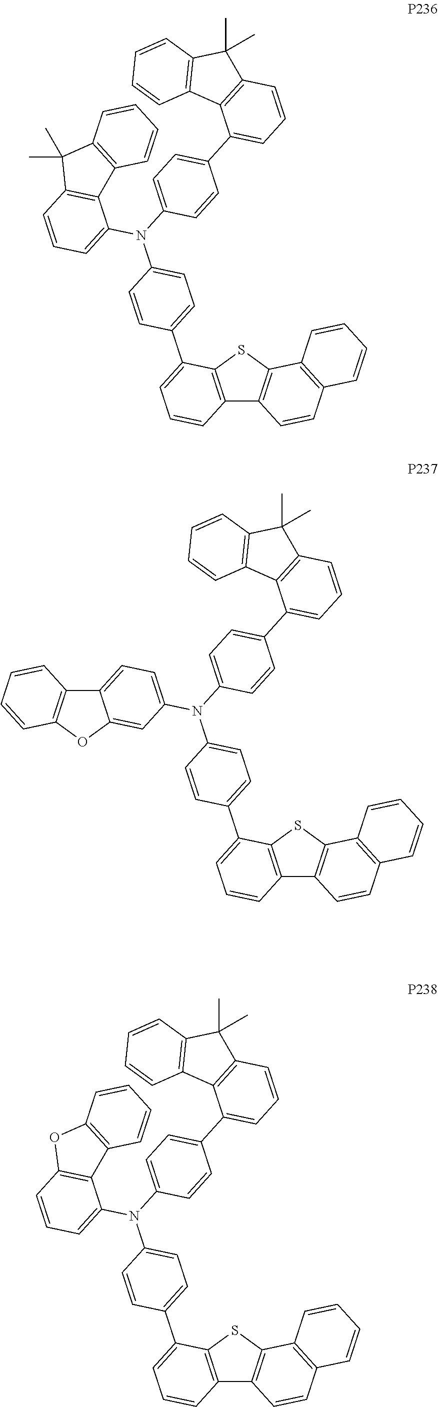 [SMILES: CC1(C)c2ccccc2-c2c(-c3ccc(N(c4ccc(-c5cccc6c5sc5c7ccccc7ccc65)cc4)c4cccc5c4-c4ccccc4C5(C)C)cc3)cccc21 ; CC1(C)c2ccccc2-c2c(-c3ccc(N(c4ccc(-c5cccc6c5sc5c7ccccc7ccc65)cc4)c4ccc5c(c4)oc4ccccc45)cc3)cccc21 ; CC1(C)c2ccccc2-c2c(-c3ccc(N(c4ccc(-c5cccc6c5sc5c7ccccc7ccc65)cc4)c4cccc5oc6ccccc6c45)cc3)cccc21]

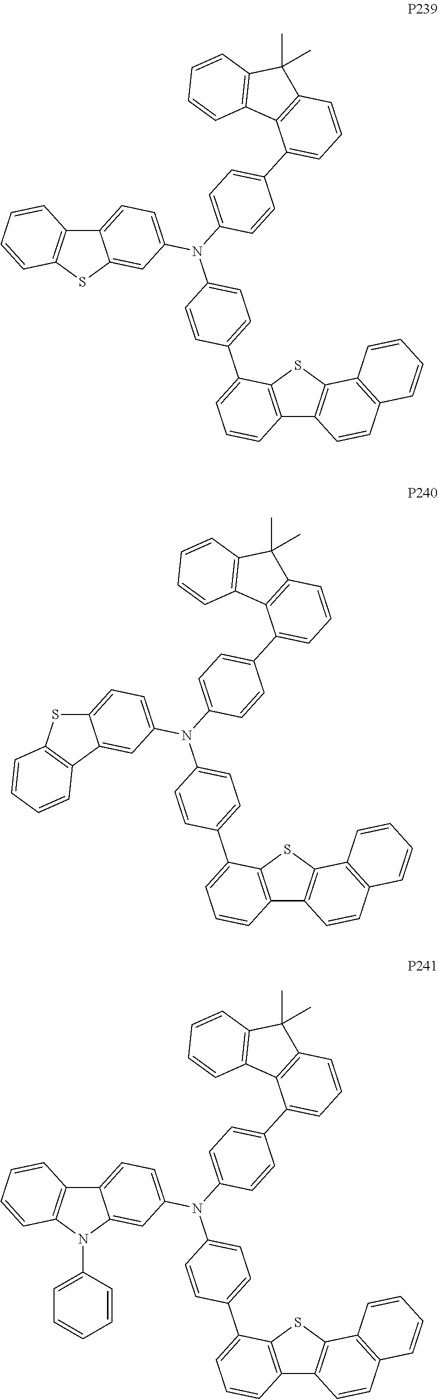 [SMILES: CC1(C)c2ccccc2-c2c(-c3ccc(N(c4ccc(-c5cccc6c5sc5c7ccccc7ccc65)cc4)c4ccc5c(c4)sc4ccccc45)cc3)cccc21 ; CC1(C)c2ccccc2-c2c(-c3ccc(N(c4ccc(-c5cccc6c5sc5c7ccccc7ccc65)cc4)c4ccc5sc6ccccc6c5c4)cc3)cccc21 ; CC1(C)c2ccccc2-c2c(-c3ccc(N(c4ccc(-c5cccc6c5sc5c7ccccc7ccc65)cc4)c4ccc5c6ccccc6n(-c6ccccc6)c5c4)cc3)cccc21]

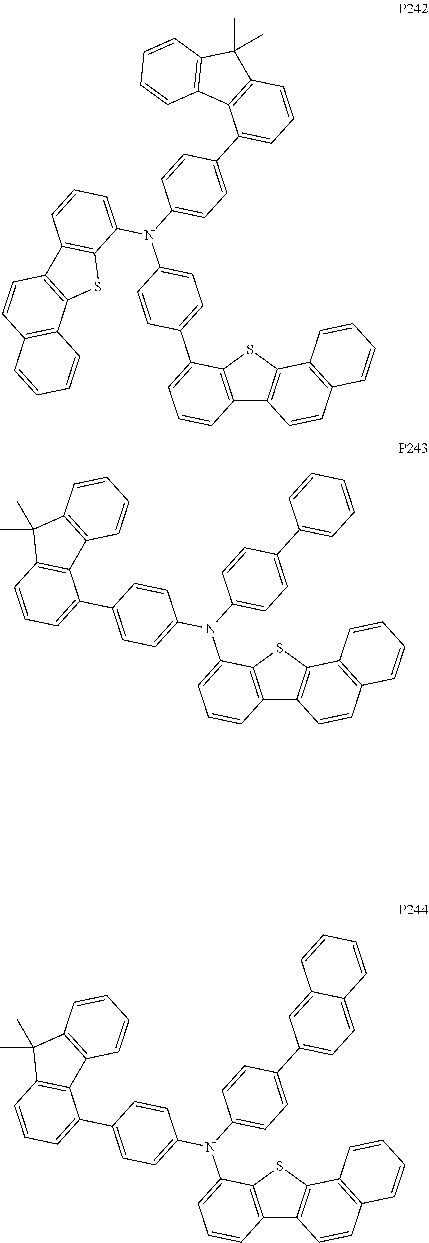 [SMILES: CC1(C)c2ccccc2-c2c(-c3ccc(N(c4ccc(-c5cccc6c5sc5c7ccccc7ccc65)cc4)c4cccc5c4sc4c6ccccc6ccc54)cc3)cccc21 ; CC1(C)c2ccccc2-c2c(-c3ccc(N(c4ccc(-c5ccccc5)cc4)c4cccc5c4sc4c6ccccc6ccc54)cc3)cccc21 ; CC1(C)c2ccccc2-c2c(-c3ccc(N(c4ccc(-c5ccc6ccccc6c5)cc4)c4cccc5c4sc4c6ccccc6ccc54)cc3)cccc21]

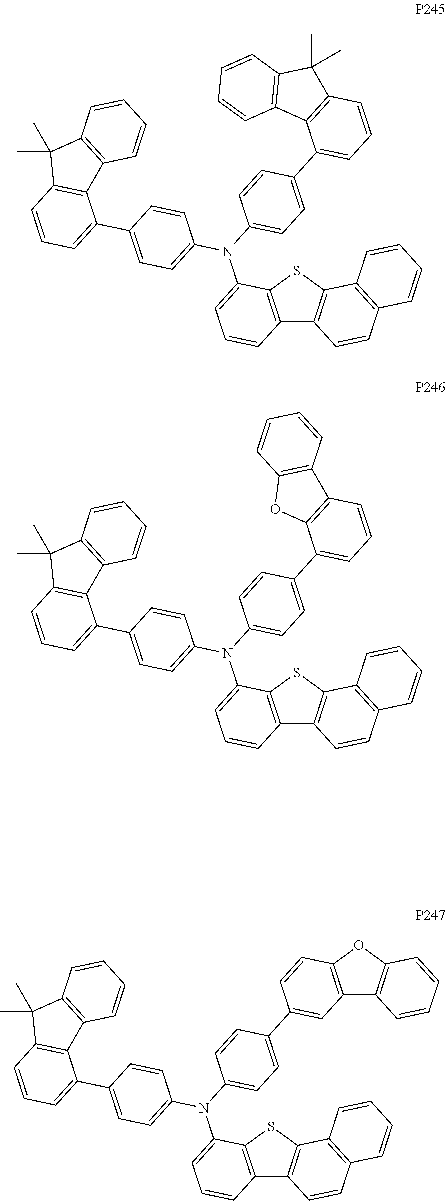 [SMILES: CC1(C)c2ccccc2-c2c(-c3ccc(N(c4ccc(-c5cccc6c5-c5ccccc5C6(C)C)cc4)c4cccc5c4sc4c6ccccc6ccc54)cc3)cccc21 ; CC1(C)c2ccccc2-c2c(-c3ccc(N(c4ccc(-c5cccc6c5oc5ccccc56)cc4)c4cccc5c4sc4c6ccccc6ccc54)cc3)cccc21 ; CC1(C)c2ccccc2-c2c(-c3ccc(N(c4ccc(-c5ccc6oc7ccccc7c6c5)cc4)c4cccc5c4sc4c6ccccc6ccc54)cc3)cccc21]

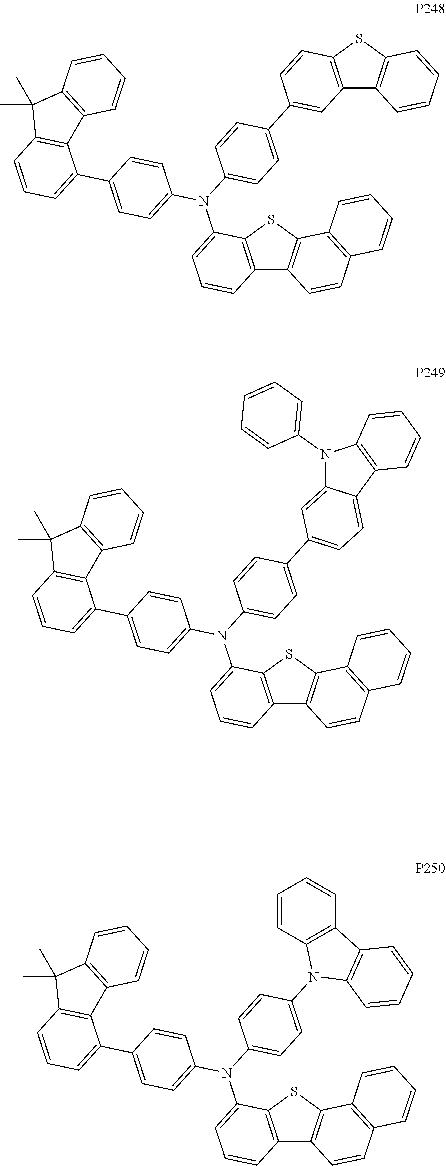 [SMILES: CC1(C)c2ccccc2-c2c(-c3ccc(N(c4ccc(-c5ccc6sc7ccccc7c6c5)cc4)c4cccc5c4sc4c6ccccc6ccc54)cc3)cccc21 ; CC1(C)c2ccccc2-c2c(-c3ccc(N(c4ccc(-c5ccc6c7ccccc7n(-c7ccccc7)c6c5)cc4)c4cccc5c4sc4c6ccccc6ccc54)cc3)cccc21 ; CC1(C)c2ccccc2-c2c(-c3ccc(N(c4ccc(-n5c6ccccc6c6ccccc65)cc4)c4cccc5c4sc4c6ccccc6ccc54)cc3)cccc21]

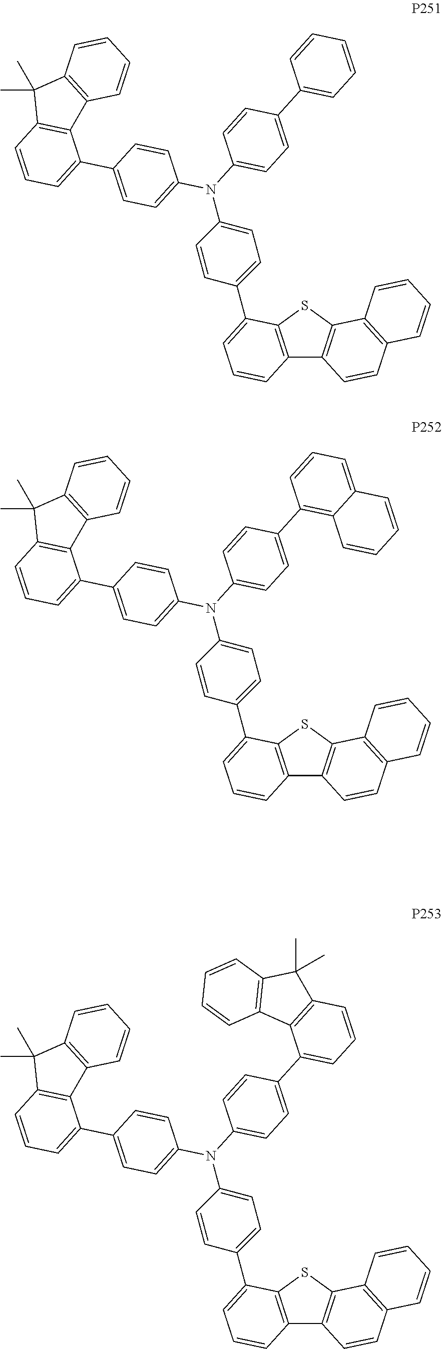 [SMILES: CC1(C)c2ccccc2-c2c(-c3ccc(N(c4ccc(-c5ccccc5)cc4)c4ccc(-c5cccc6c5sc5c7ccccc7ccc65)cc4)cc3)cccc21 ; CC1(C)c2ccccc2-c2c(-c3ccc(N(c4ccc(-c5cccc6ccccc56)cc4)c4ccc(-c5cccc6c5sc5c7ccccc7ccc65)cc4)cc3)cccc21 ; CC1(C)c2ccccc2-c2c(-c3ccc(N(c4ccc(-c5cccc6c5-c5ccccc5C6(C)C)cc4)c4ccc(-c5cccc6c5sc5c7ccccc7ccc65)cc4)cc3)cccc21]

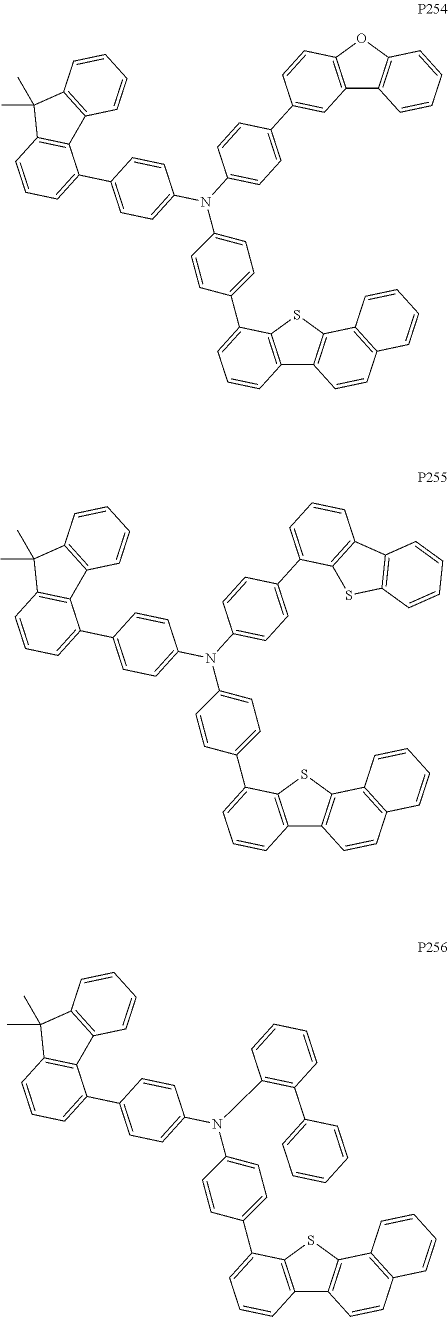 [SMILES: CC1(C)c2ccccc2-c2c(-c3ccc(N(c4ccc(-c5ccc6oc7ccccc7c6c5)cc4)c4ccc(-c5cccc6c5sc5c7ccccc7ccc65)cc4)cc3)cccc21 ; CC1(C)c2ccccc2-c2c(-c3ccc(N(c4ccc(-c5cccc6c5sc5ccccc56)cc4)c4ccc(-c5cccc6c5sc5c7ccccc7ccc65)cc4)cc3)cccc21 ; CC1(C)c2ccccc2-c2c(-c3ccc(N(c4ccc(-c5cccc6c5sc5c7ccccc7ccc65)cc4)c4ccccc4-c4ccccc4)cc3)cccc21]

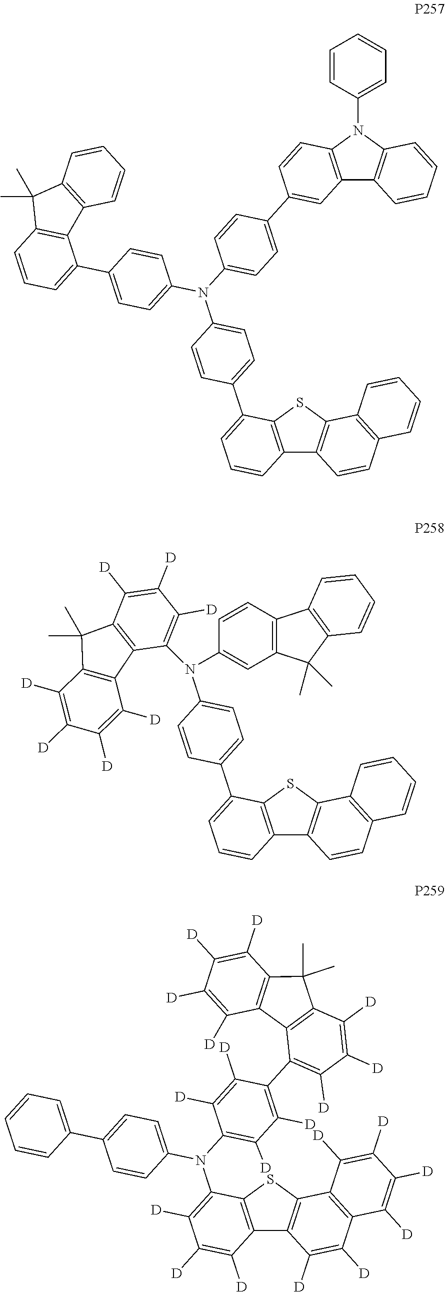 [SMILES: CC1(C)c2ccccc2-c2c(-c3ccc(N(c4ccc(-c5ccc6c(c5)c5ccccc5n6-c5ccccc5)cc4)c4ccc(-c5cccc6c5sc5c7ccccc7ccc65)cc4)cc3)cccc21 ; [2H]c1c([2H])c([2H])c2c(c1[2H])-c1c(N(c3ccc(-c4cccc5c4sc4c6ccccc6ccc54)cc3)c3ccc4c(c3)C(C)(C)c3ccccc3-4)c([2H])c([2H])c([2H])c1C2(C)C ; [2H]c1c([2H])c([2H])c2c(c1[2H])-c1c(-c3c([2H])c([2H])c(N(c4ccc(-c5ccccc5)cc4)c4c([2H])c([2H])c([2H])c5c4sc4c6c([2H])c([2H])c([2H])c([2H])c6c([2H])c([2H])c45)c([2H])c3[2H])c([2H])c([2H])c([2H])c1C2(C)C]

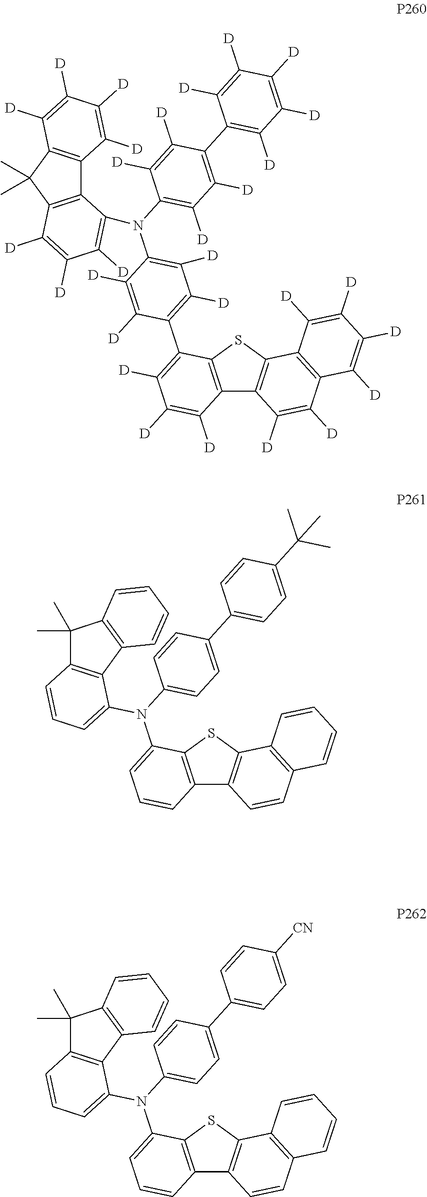 [SMILES: [2H]c1c([2H])c([2H])c(-c2c([2H])c([2H])c(N(c3c([2H])c([2H])c(-c4c([2H])c([2H])c([2H])c5c4sc4c6c([2H])c([2H])c([2H])c([2H])c6c([2H])c([2H])c45)c([2H])c3[2H])c3c([2H])c([2H])c([2H])c4c3-c3c([2H])c([2H])c([2H])c([2H])c3C4(C)C)c([2H])c2[2H])c([2H])c1[2H] ; CC(C)(C)c1ccc(-c2ccc(N(c3cccc4c3-c3ccccc3C4(C)C)c3cccc4c3sc3c5ccccc5ccc43)cc2)cc1 ; CC1(C)c2ccccc2-c2c(N(c3ccc(-c4ccc(C#N)cc4)cc3)c3cccc4c3sc3c5ccccc5ccc43)cccc21]

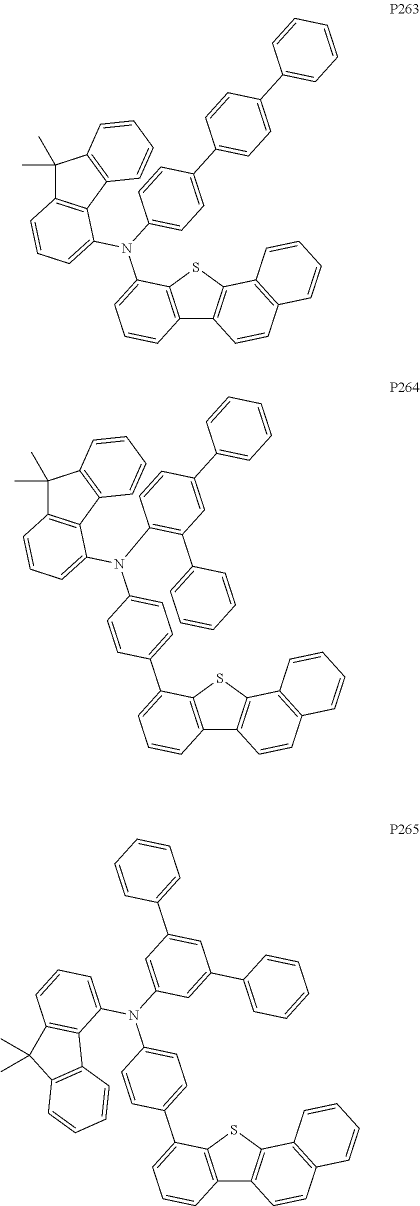 [SMILES: CC1(C)c2ccccc2-c2c(N(c3ccc(-c4ccc(-c5ccccc5)cc4)cc3)c3cccc4c3sc3c5ccccc5ccc43)cccc21 ; CC1(C)c2ccccc2-c2c(N(c3ccc(-c4cccc5c4sc4c6ccccc6ccc54)cc3)c3ccc(-c4ccccc4)cc3-c3ccccc3)cccc21 ; CC1(C)c2ccccc2-c2c(N(c3ccc(-c4cccc5c4sc4c6ccccc6ccc54)cc3)c3cc(-c4ccccc4)cc(-c4ccccc4)c3)cccc21]

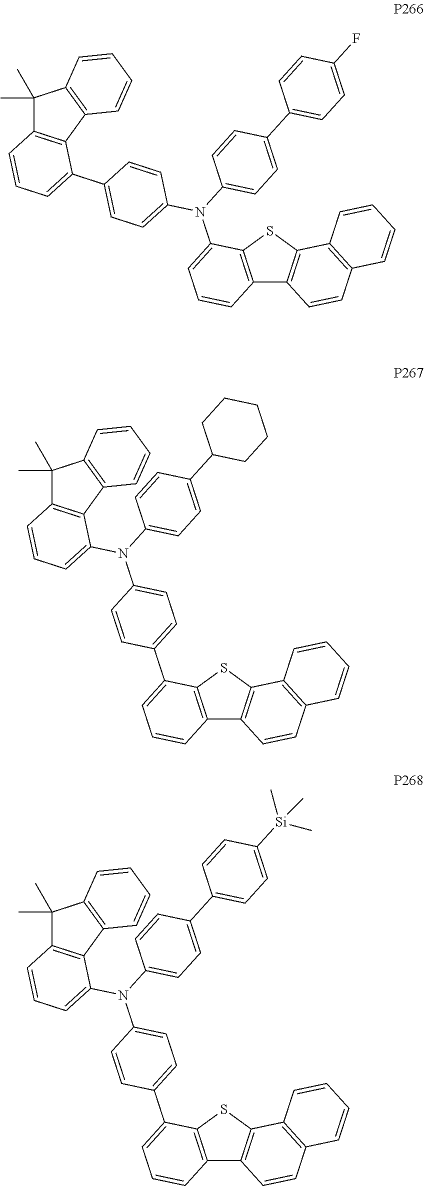

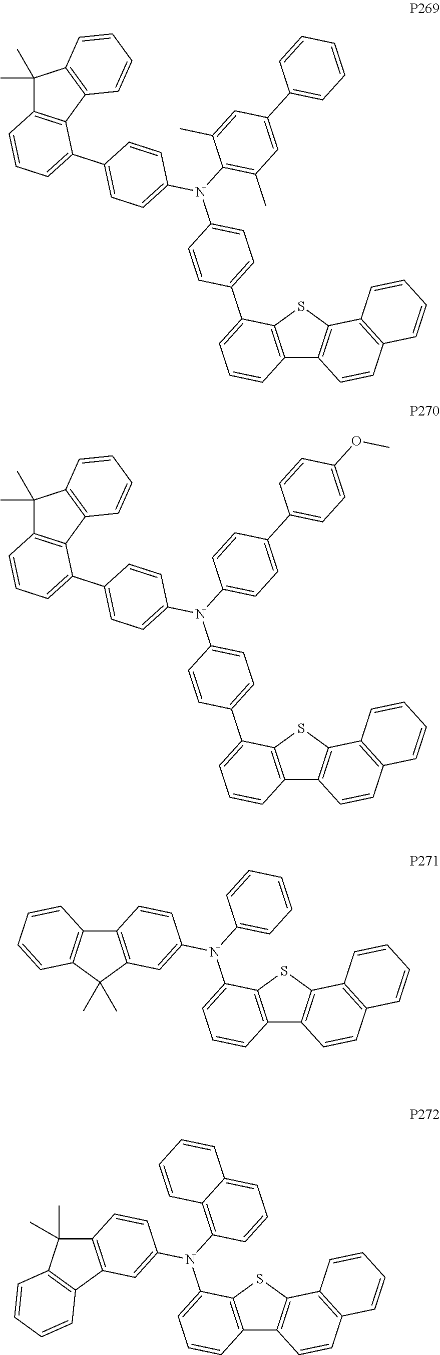 [SMILES: Cc1cc(-c2ccccc2)cc(C)c1N(c1ccc(-c2cccc3c2-c2ccccc2C3(C)C)cc1)c1ccc(-c2cccc3c2sc2c4ccccc4ccc32)cc1 ; COc1ccc(-c2ccc(N(c3ccc(-c4cccc5c4-c4ccccc4C5(C)C)cc3)c3ccc(-c4cccc5c4sc4c6ccccc6ccc54)cc3)cc2)cc1 ; CC1(C)c2ccccc2-c2ccc(N(c3ccccc3)c3cccc4c3sc3c5ccccc5ccc43)cc21 ; CC1(C)c2ccccc2-c2cc(N(c3cccc4ccccc34)c3cccc4c3sc3c5ccccc5ccc43)ccc21]

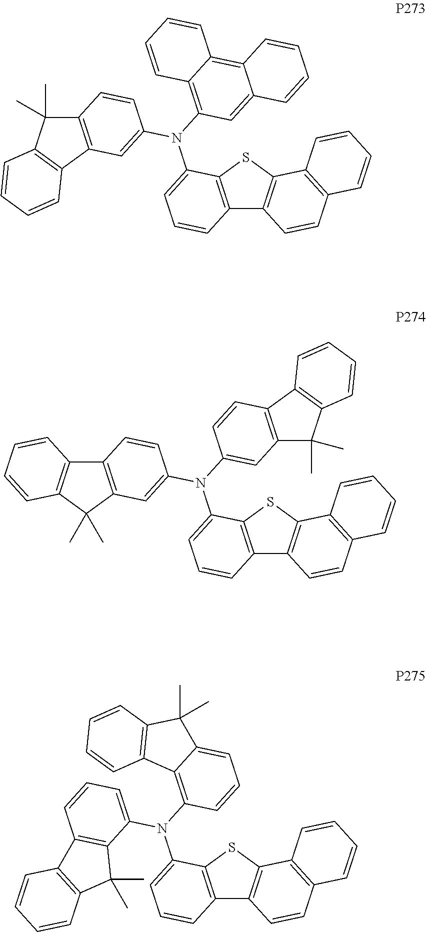 [SMILES: CC1(C)c2ccccc2-c2cc(N(c3cc4ccccc4c4ccccc34)c3cccc4c3sc3c5ccccc5ccc43)ccc21 ; CC1(C)c2ccccc2-c2ccc(N(c3ccc4c(c3)C(C)(C)c3ccccc3-4)c3cccc4c3sc3c5ccccc5ccc43)cc21 ; CC1(C)c2ccccc2-c2c(N(c3cccc4c3C(C)(C)c3ccccc3-4)c3cccc4c3sc3c5ccccc5ccc43)cccc21]

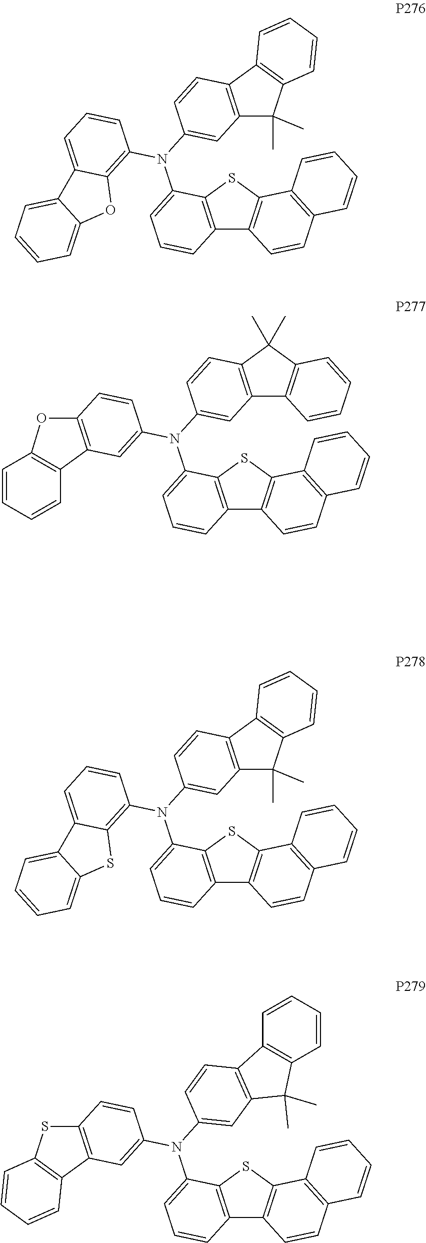 [SMILES: CC1(C)c2ccccc2-c2ccc(N(c3cccc4c3oc3ccccc34)c3cccc4c3sc3c5ccccc5ccc43)cc21 ; CC1(C)c2ccccc2-c2cc(N(c3ccc4oc5ccccc5c4c3)c3cccc4c3sc3c5ccccc5ccc43)ccc21 ; CC1(C)c2ccccc2-c2ccc(N(c3cccc4c3sc3ccccc34)c3cccc4c3sc3c5ccccc5ccc43)cc21 ; CC1(C)c2ccccc2-c2ccc(N(c3ccc4sc5ccccc5c4c3)c3cccc4c3sc3c5ccccc5ccc43)cc21]

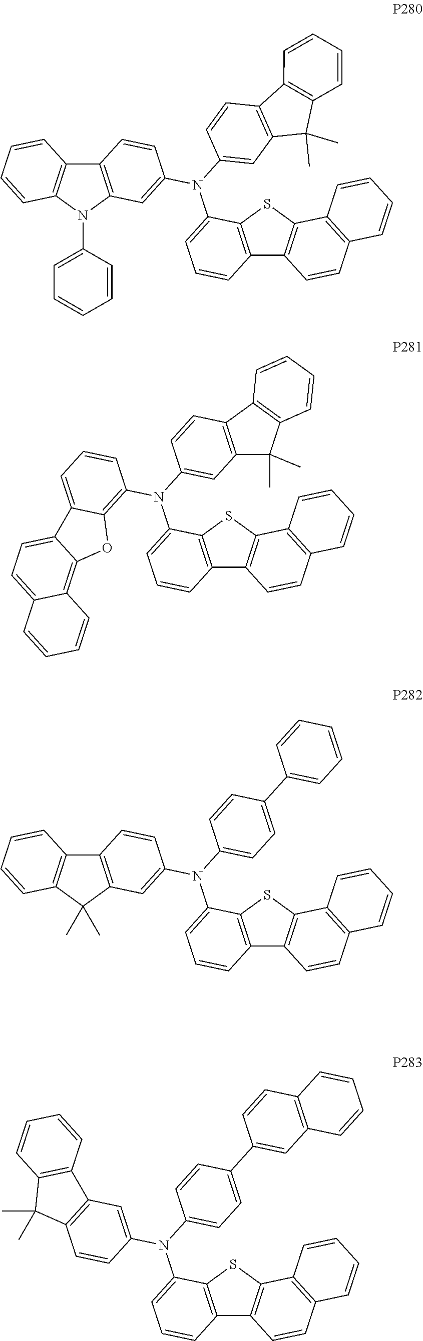 [SMILES: CC1(C)c2ccccc2-c2ccc(N(c3ccc4c5ccccc5n(-c5ccccc5)c4c3)c3cccc4c3sc3c5ccccc5ccc43)cc21 ; CC1(C)c2ccccc2-c2ccc(N(c3cccc4c3oc3c5ccccc5ccc43)c3cccc4c3sc3c5ccccc5ccc43)cc21 ; CC1(C)c2ccccc2-c2ccc(N(c3ccc(-c4ccccc4)cc3)c3cccc4c3sc3c5ccccc5ccc43)cc21 ; CC1(C)c2ccccc2-c2cc(N(c3ccc(-c4ccc5ccccc5c4)cc3)c3cccc4c3sc3c5ccccc5ccc43)ccc21]

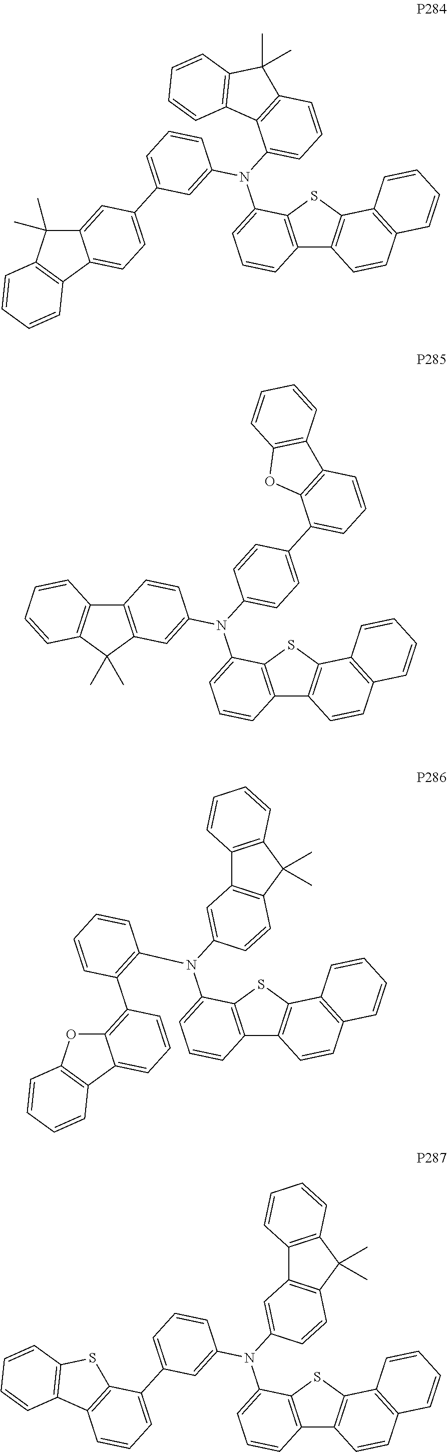 [SMILES: CC1(C)c2ccccc2-c2ccc(-c3cccc(N(c4cccc5c4-c4ccccc4C5(C)C)c4cccc5c4sc4c6ccccc6ccc54)c3)cc21 ; CC1(C)c2ccccc2-c2ccc(N(c3ccc(-c4cccc5c4oc4ccccc45)cc3)c3cccc4c3sc3c5ccccc5ccc43)cc21 ; CC1(C)c2ccccc2-c2cc(N(c3ccccc3-c3cccc4c3oc3ccccc34)c3cccc4c3sc3c5ccccc5ccc43)ccc21 ; CC1(C)c2ccccc2-c2cc(N(c3cccc(-c4cccc5c4sc4ccccc45)c3)c3cccc4c3sc3c5ccccc5ccc43)ccc21]

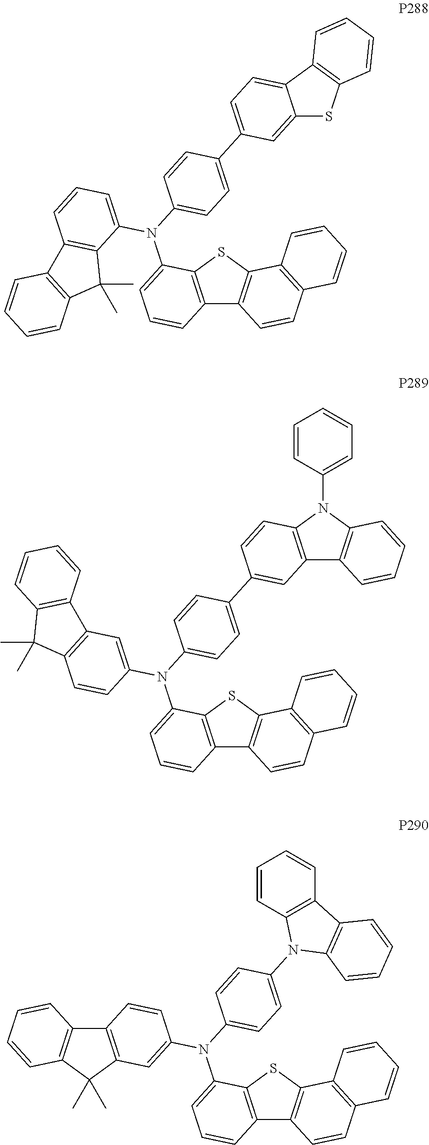 [SMILES: CC1(C)c2ccccc2-c2cccc(N(c3ccc(-c4ccc5c(c4)sc4ccccc45)cc3)c3cccc4c3sc3c5ccccc5ccc43)c21 ; CC1(C)c2ccccc2-c2cc(N(c3ccc(-c4ccc5c(c4)c4ccccc4n5-c4ccccc4)cc3)c3cccc4c3sc3c5ccccc5ccc43)ccc21 ; CC1(C)c2ccccc2-c2ccc(N(c3ccc(-n4c5ccccc5c5ccccc54)cc3)c3cccc4c3sc3c5ccccc5ccc43)cc21]

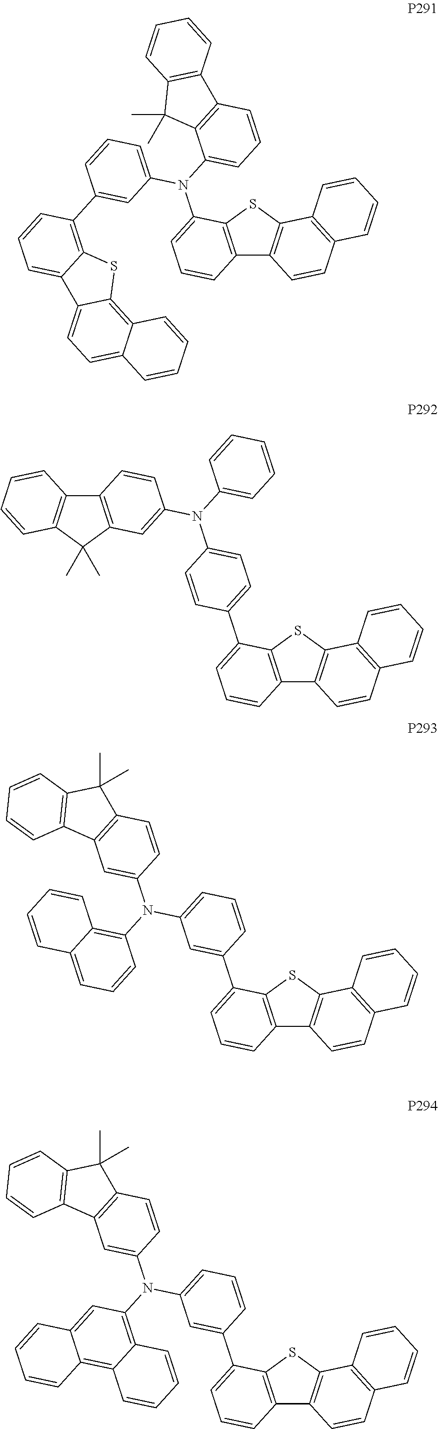 [SMILES: CC1(C)c2ccccc2-c2cccc(N(c3cccc(-c4cccc5c4sc4c6ccccc6ccc54)c3)c3cccc4c3sc3c5ccccc5ccc43)c21 ; CC1(C)c2ccccc2-c2ccc(N(c3ccccc3)c3ccc(-c4cccc5c4sc4c6ccccc6ccc54)cc3)cc21 ; CC1(C)c2ccccc2-c2cc(N(c3cccc(-c4cccc5c4sc4c6ccccc6ccc54)c3)c3cccc4ccccc34)ccc21 ; CC1(C)c2ccccc2-c2cc(N(c3cccc(-c4cccc5c4sc4c6ccccc6ccc54)c3)c3cc4ccccc4c4ccccc34)ccc21]

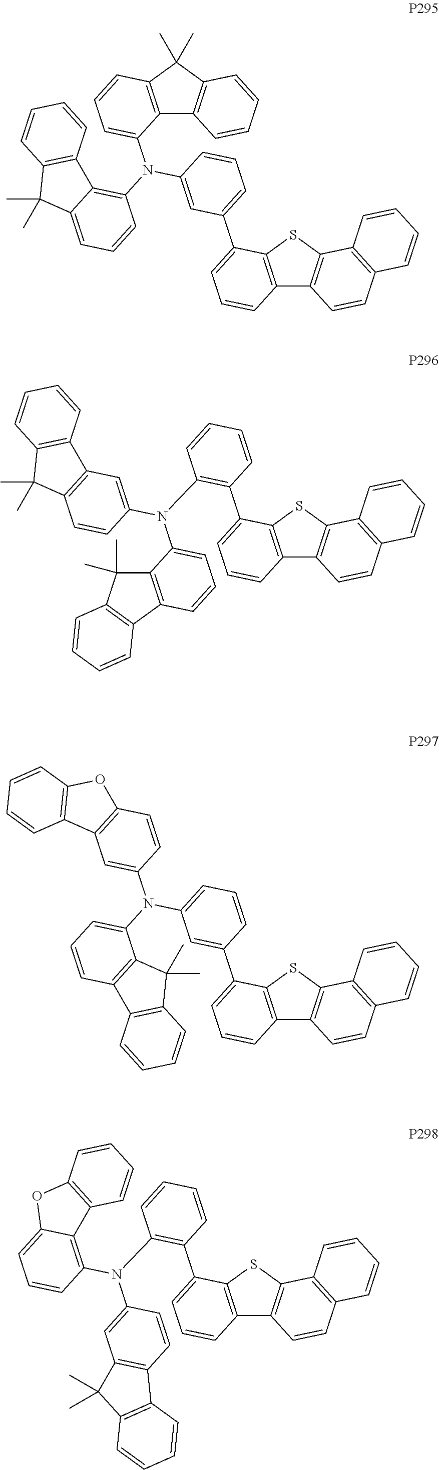 [SMILES: CC1(C)c2ccccc2-c2c(N(c3cccc(-c4cccc5c4sc4c6ccccc6ccc54)c3)c3cccc4c3-c3ccccc3C4(C)C)cccc21 ; CC1(C)c2ccccc2-c2cc(N(c3ccccc3-c3cccc4c3sc3c5ccccc5ccc43)c3cccc4c3C(C)(C)c3ccccc3-4)ccc21 ; CC1(C)c2ccccc2-c2cccc(N(c3cccc(-c4cccc5c4sc4c6ccccc6ccc54)c3)c3ccc4oc5ccccc5c4c3)c21 ; CC1(C)c2ccccc2-c2ccc(N(c3ccccc3-c3cccc4c3sc3c5ccccc5ccc43)c3cccc4oc5ccccc5c34)cc21]

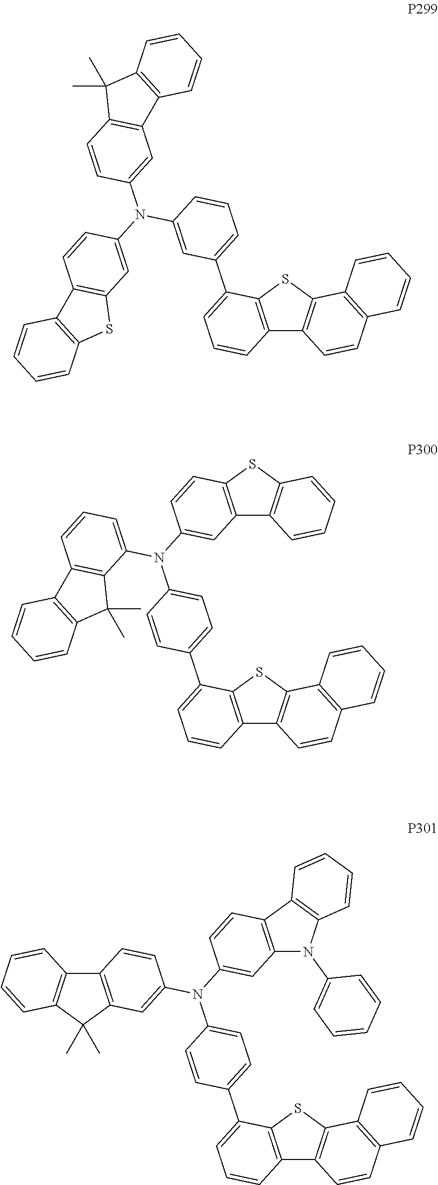 [SMILES: CC1(C)c2ccccc2-c2cc(N(c3cccc(-c4cccc5c4sc4c6ccccc6ccc54)c3)c3ccc4c(c3)sc3ccccc34)ccc21 ; CC1(C)c2ccccc2-c2cccc(N(c3ccc(-c4cccc5c4sc4c6ccccc6ccc54)cc3)c3ccc4sc5ccccc5c4c3)c21 ; CC1(C)c2ccccc2-c2ccc(N(c3ccc(-c4cccc5c4sc4c6ccccc6ccc54)cc3)c3ccc4c5ccccc5n(-c5ccccc5)c4c3)cc21]

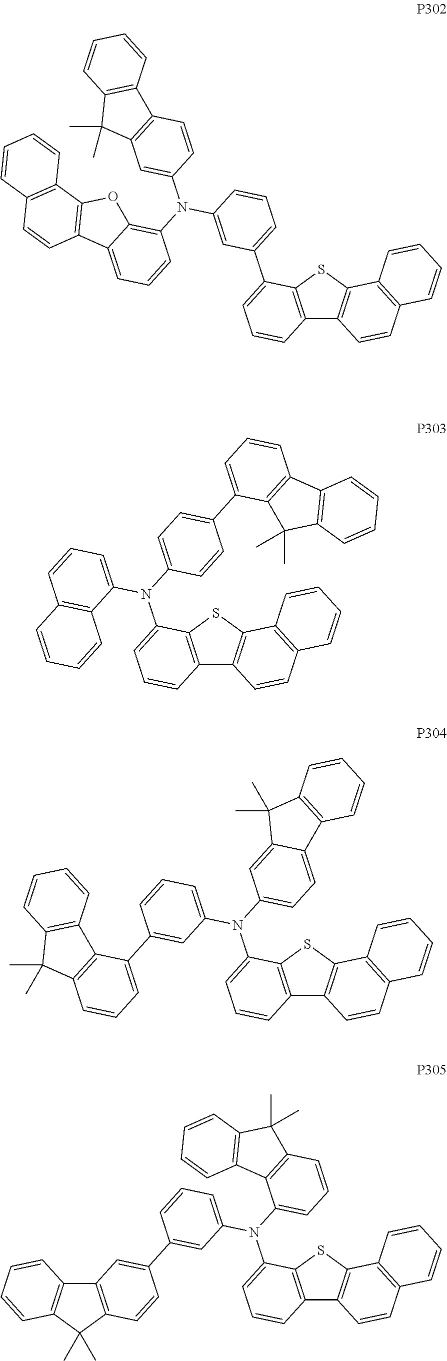 [SMILES: CC1(C)c2ccccc2-c2ccc(N(c3cccc(-c4cccc5c4sc4c6ccccc6ccc54)c3)c3cccc4c3oc3c5ccccc5ccc43)cc21 ; CC1(C)c2ccccc2-c2cccc(-c3ccc(N(c4cccc5ccccc45)c4cccc5c4sc4c6ccccc6ccc54)cc3)c21 ; CC1(C)c2ccccc2-c2ccc(N(c3cccc(-c4cccc5c4-c4ccccc4C5(C)C)c3)c3cccc4c3sc3c5ccccc5ccc43)cc21 ; CC1(C)c2ccccc2-c2cc(-c3cccc(N(c4cccc5c4-c4ccccc4C5(C)C)c4cccc5c4sc4c6ccccc6ccc54)c3)ccc21]

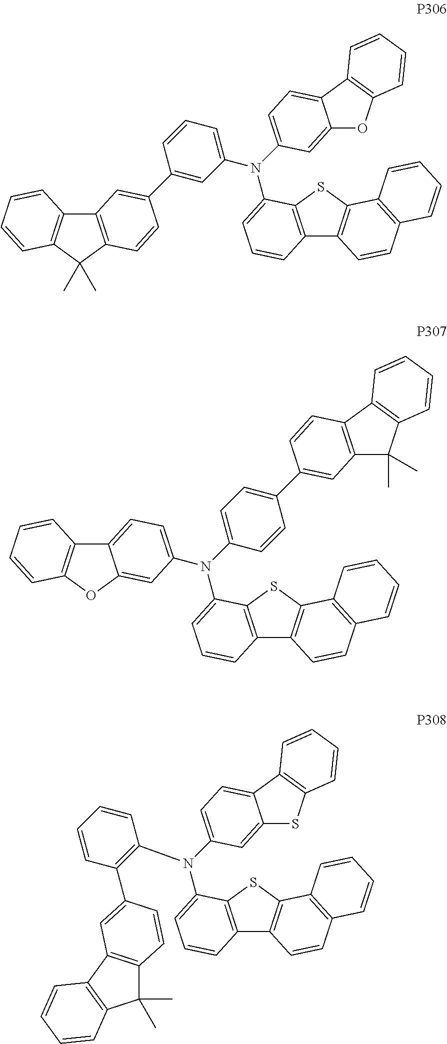 [SMILES: CC1(C)c2ccccc2-c2cc(-c3cccc(N(c4ccc5c(c4)oc4ccccc45)c4cccc5c4sc4c6ccccc6ccc54)c3)ccc21 ; CC1(C)c2ccccc2-c2ccc(-c3ccc(N(c4ccc5c(c4)oc4ccccc45)c4cccc5c4sc4c6ccccc6ccc54)cc3)cc21 ; CC1(C)c2ccccc2-c2cc(-c3ccccc3N(c3ccc4c(c3)sc3ccccc34)c3cccc4c3sc3c5ccccc5ccc43)ccc21]

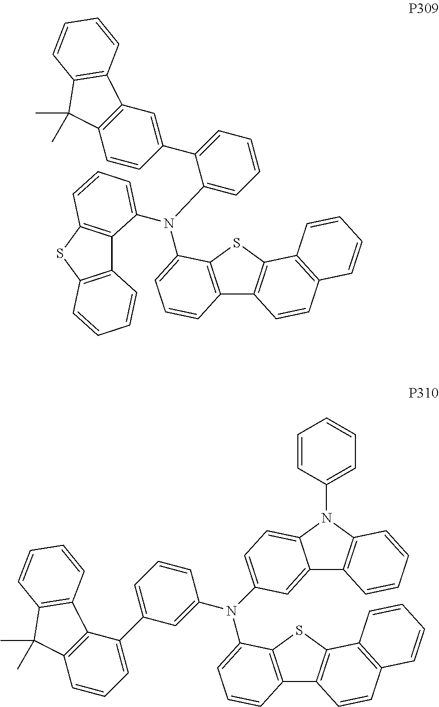 [SMILES: CC1(C)c2ccccc2-c2cc(-c3ccccc3N(c3cccc4c3sc3c5ccccc5ccc43)c3cccc4sc5ccccc5c34)ccc21 ; CC1(C)c2ccccc2-c2c(-c3cccc(N(c4ccc5c(c4)c4ccccc4n5-c4ccccc4)c4cccc5c4sc4c6ccccc6ccc54)c3)cccc21]

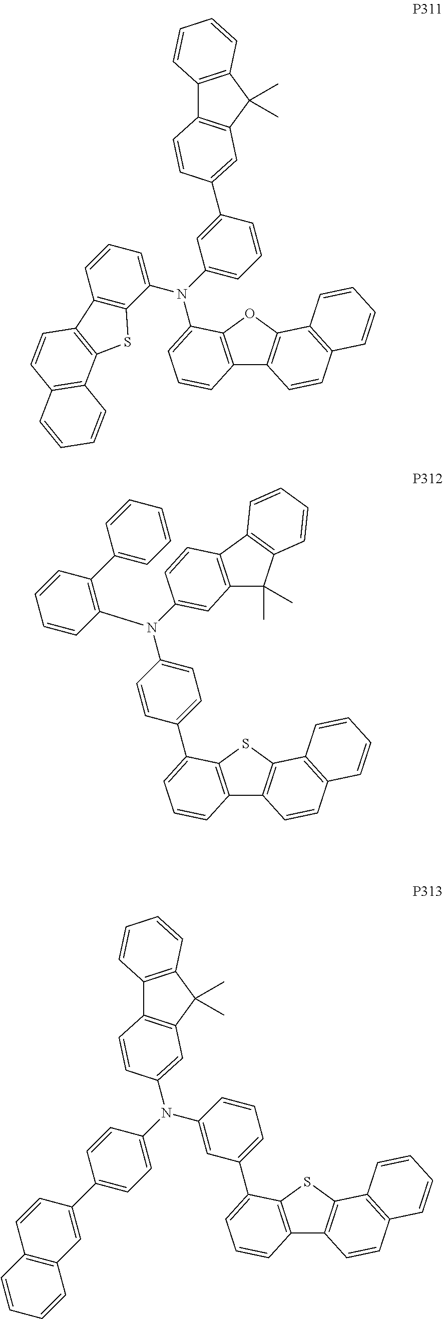 [SMILES: CC1(C)c2ccccc2-c2ccc(-c3cccc(N(c4cccc5c4oc4c6ccccc6ccc54)c4cccc5c4sc4c6ccccc6ccc54)c3)cc21 ; CC1(C)c2ccccc2-c2ccc(N(c3ccc(-c4cccc5c4sc4c6ccccc6ccc54)cc3)c3ccccc3-c3ccccc3)cc21 ; CC1(C)c2ccccc2-c2ccc(N(c3ccc(-c4ccc5ccccc5c4)cc3)c3cccc(-c4cccc5c4sc4c6ccccc6ccc54)c3)cc21]

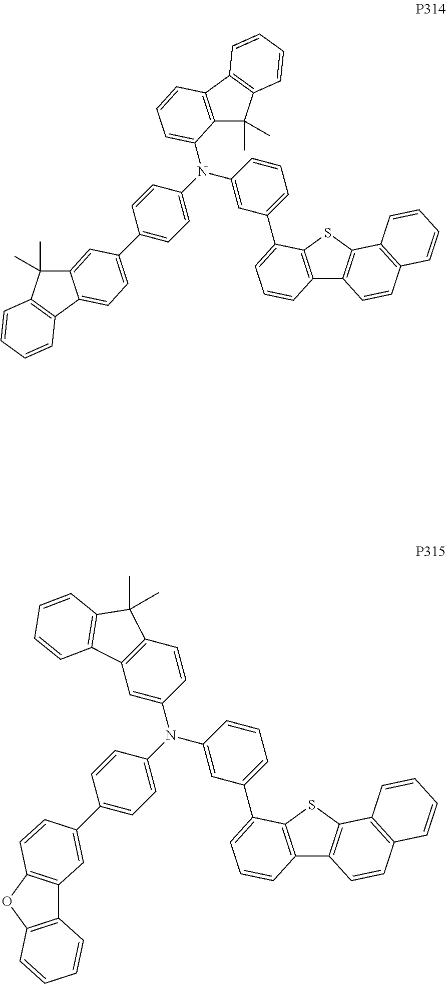 [SMILES: CC1(C)c2ccccc2-c2ccc(-c3ccc(N(c4cccc(-c5cccc6c5sc5c7ccccc7ccc65)c4)c4cccc5c4C(C)(C)c4ccccc4-5)cc3)cc21 ; CC1(C)c2ccccc2-c2cc(N(c3ccc(-c4ccc5oc6ccccc6c5c4)cc3)c3cccc(-c4cccc5c4sc4c6ccccc6ccc54)c3)ccc21]

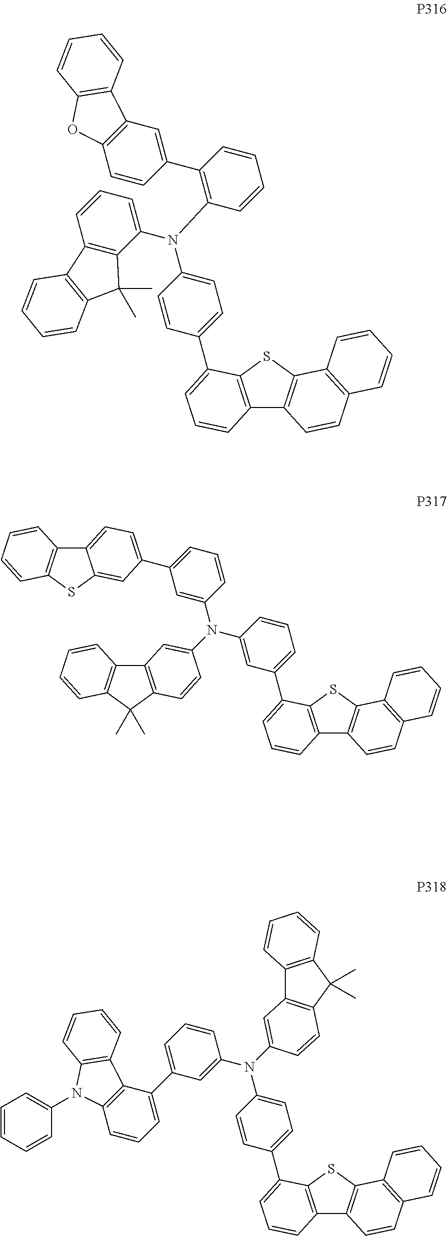 [SMILES: CC1(C)c2ccccc2-c2cccc(N(c3ccc(-c4cccc5c4sc4c6ccccc6ccc54)cc3)c3ccccc3-c3ccc4oc5ccccc5c4c3)c21 ; CC1(C)c2ccccc2-c2cc(N(c3cccc(-c4ccc5c(c4)sc4ccccc45)c3)c3cccc(-c4cccc5c4sc4c6ccccc6ccc54)c3)ccc21 ; CC1(C)c2ccccc2-c2cc(N(c3ccc(-c4cccc5c4sc4c6ccccc6ccc54)cc3)c3cccc(-c4cccc5c4c4ccccc4n5-c4ccccc4)c3)ccc21]

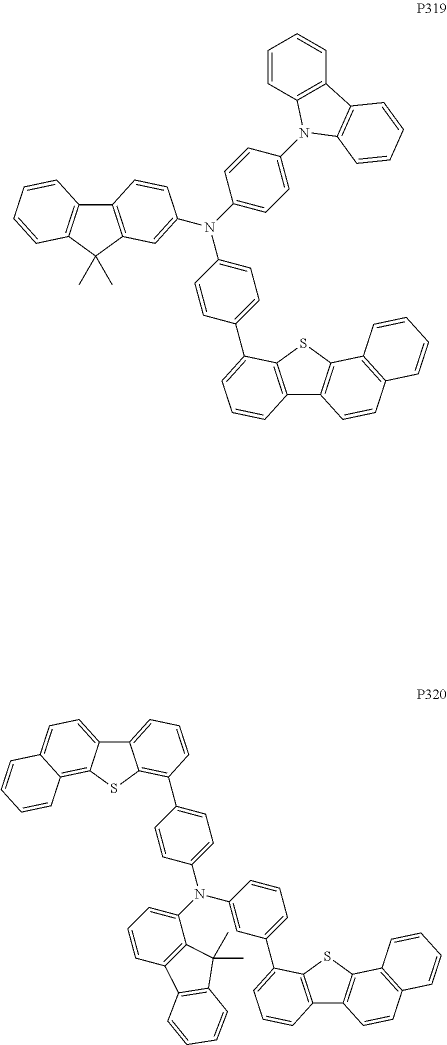 [SMILES: CC1(C)c2ccccc2-c2ccc(N(c3ccc(-c4cccc5c4sc4c6ccccc6ccc54)cc3)c3ccc(-n4c5ccccc5c5ccccc54)cc3)cc21 ; CC1(C)c2ccccc2-c2cccc(N(c3ccc(-c4cccc5c4sc4c6ccccc6ccc54)cc3)c3cccc(-c4cccc5c4sc4c6ccccc6ccc54)c3)c21]

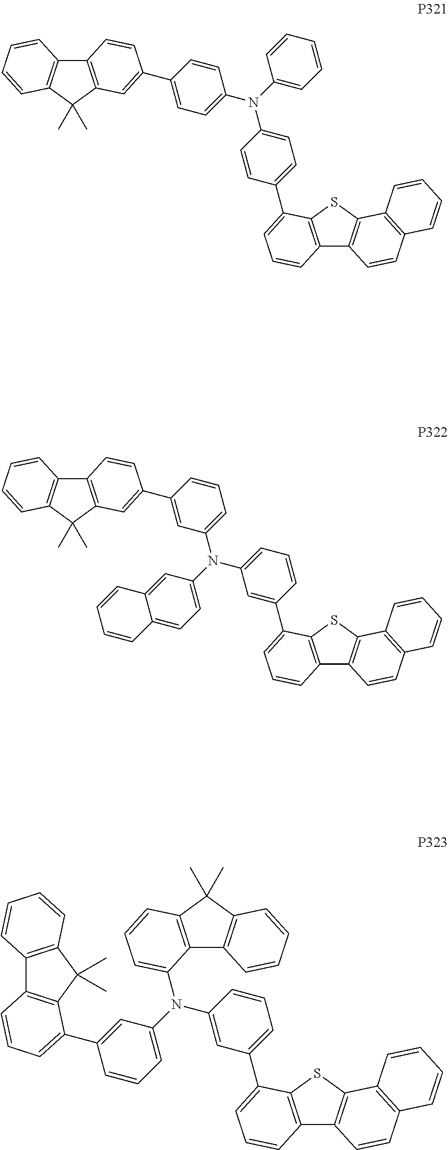 [SMILES: CC1(C)c2ccccc2-c2ccc(-c3ccc(N(c4ccccc4)c4ccc(-c5cccc6c5sc5c7ccccc7ccc65)cc4)cc3)cc21 ; CC1(C)c2ccccc2-c2ccc(-c3cccc(N(c4cccc(-c5cccc6c5sc5c7ccccc7ccc65)c4)c4ccc5ccccc5c4)c3)cc21 ; CC1(C)c2ccccc2-c2c(N(c3cccc(-c4cccc5c4C(C)(C)c4ccccc4-5)c3)c3cccc(-c4cccc5c4sc4c6ccccc6ccc54)c3)cccc21]

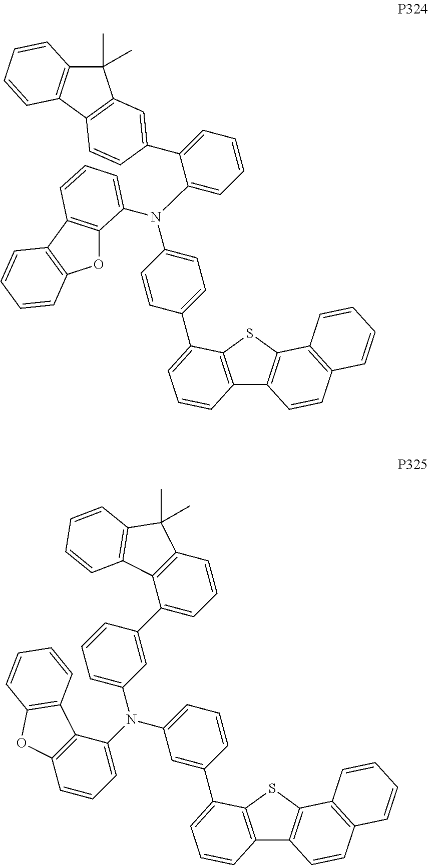 [SMILES: CC1(C)c2ccccc2-c2ccc(-c3ccccc3N(c3ccc(-c4cccc5c4sc4c6ccccc6ccc54)cc3)c3cccc4c3oc3ccccc34)cc21 ; CC1(C)c2ccccc2-c2c(-c3cccc(N(c4cccc(-c5cccc6c5sc5c7ccccc7ccc65)c4)c4cccc5oc6ccccc6c45)c3)cccc21]

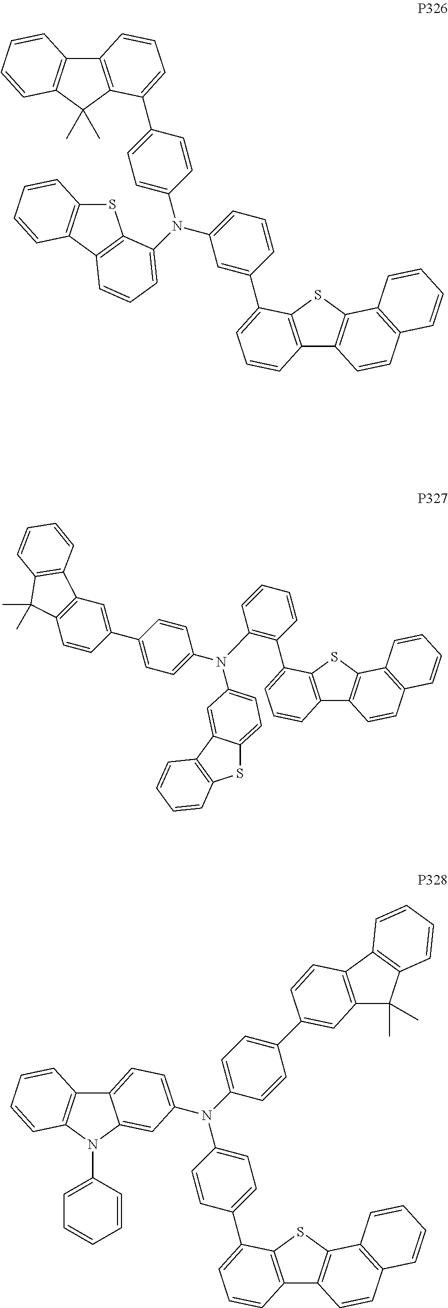 [SMILES: CC1(C)c2ccccc2-c2cccc(-c3ccc(N(c4cccc(-c5cccc6c5sc5c7ccccc7ccc65)c4)c4cccc5c4sc4ccccc45)cc3)c21 ; CC1(C)c2ccccc2-c2cc(-c3ccc(N(c4ccc5sc6ccccc6c5c4)c4ccccc4-c4cccc5c4sc4c6ccccc6ccc54)cc3)ccc21 ; CC1(C)c2ccccc2-c2ccc(-c3ccc(N(c4ccc(-c5cccc6c5sc5c7ccccc7ccc65)cc4)c4ccc5c6ccccc6n(-c6ccccc6)c5c4)cc3)cc21]

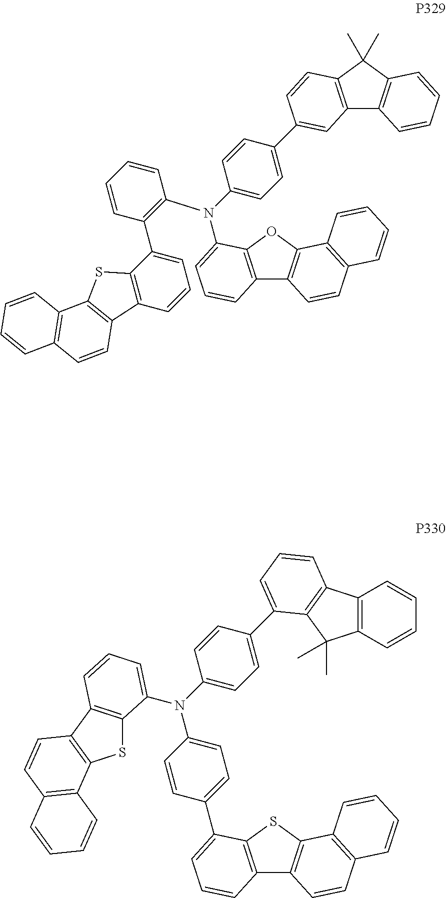 [SMILES: CC1(C)c2ccccc2-c2cc(-c3ccc(N(c4ccccc4-c4cccc5c4sc4c6ccccc6ccc54)c4cccc5c4oc4c6ccccc6ccc54)cc3)ccc21 ; CC1(C)c2ccccc2-c2cccc(-c3ccc(N(c4ccc(-c5cccc6c5sc5c7ccccc7ccc65)cc4)c4cccc5c4sc4c6ccccc6ccc54)cc3)c21]

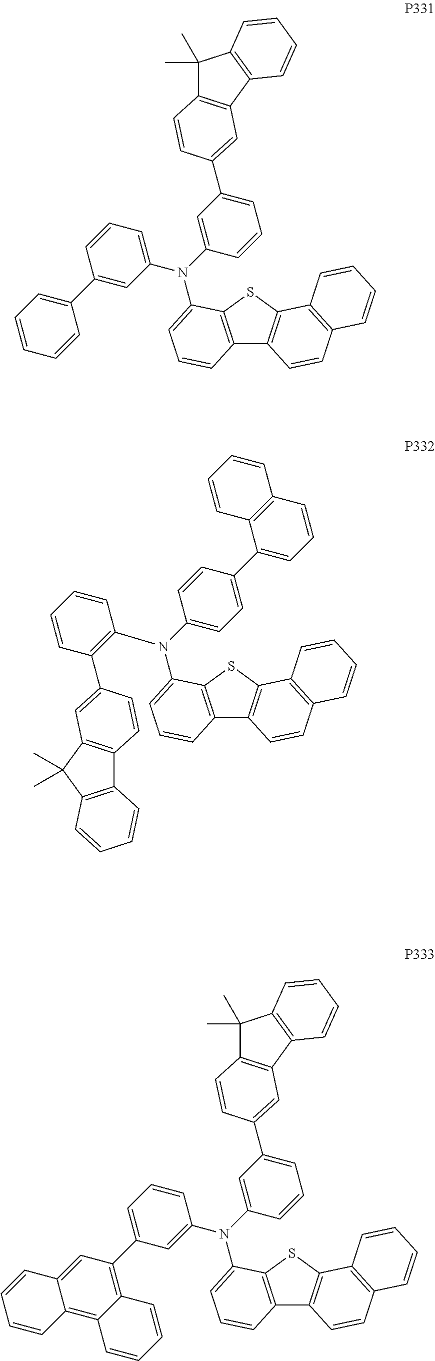 [SMILES: CC1(C)c2ccccc2-c2cc(-c3cccc(N(c4cccc(-c5ccccc5)c4)c4cccc5c4sc4c6ccccc6ccc54)c3)ccc21 ; CC1(C)c2ccccc2-c2ccc(-c3ccccc3N(c3ccc(-c4cccc5ccccc45)cc3)c3cccc4c3sc3c5ccccc5ccc43)cc21 ; CC1(C)c2ccccc2-c2cc(-c3cccc(N(c4cccc(-c5cc6ccccc6c6ccccc56)c4)c4cccc5c4sc4c6ccccc6ccc54)c3)ccc21]

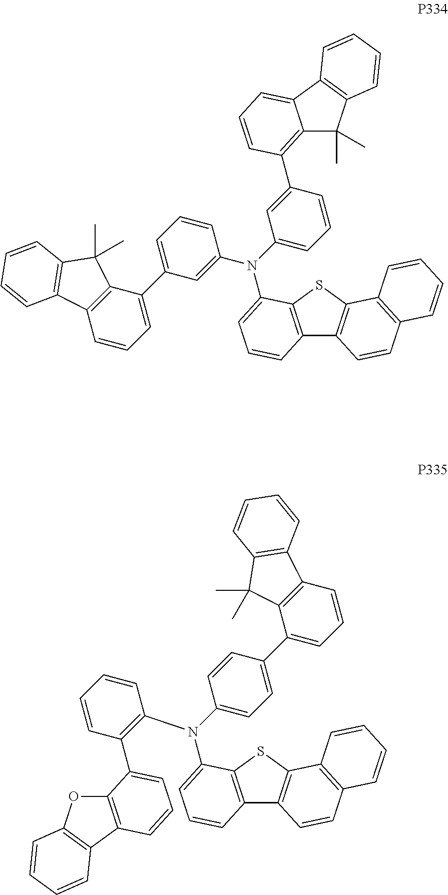 [SMILES: CC1(C)c2ccccc2-c2cccc(-c3cccc(N(c4cccc(-c5cccc6c5C(C)(C)c5ccccc5-6)c4)c4cccc5c4sc4c6ccccc6ccc54)c3)c21 ; CC1(C)c2ccccc2-c2cccc(-c3ccc(N(c4ccccc4-c4cccc5c4oc4ccccc45)c4cccc5c4sc4c6ccccc6ccc54)cc3)c21]

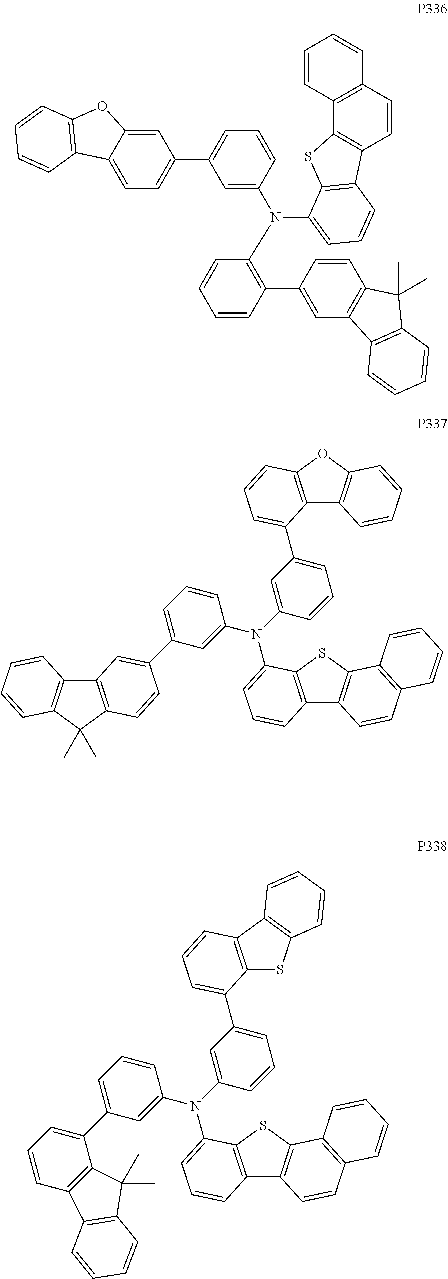 [SMILES: CC1(C)c2ccccc2-c2cc(-c3ccccc3N(c3cccc(-c4ccc5c(c4)oc4ccccc45)c3)c3cccc4c3sc3c5ccccc5ccc43)ccc21 ; CC1(C)c2ccccc2-c2cc(-c3cccc(N(c4cccc(-c5cccc6oc7ccccc7c56)c4)c4cccc5c4sc4c6ccccc6ccc54)c3)ccc21 ; CC1(C)c2ccccc2-c2cccc(-c3cccc(N(c4cccc(-c5cccc6c5sc5ccccc56)c4)c4cccc5c4sc4c6ccccc6ccc54)c3)c21]

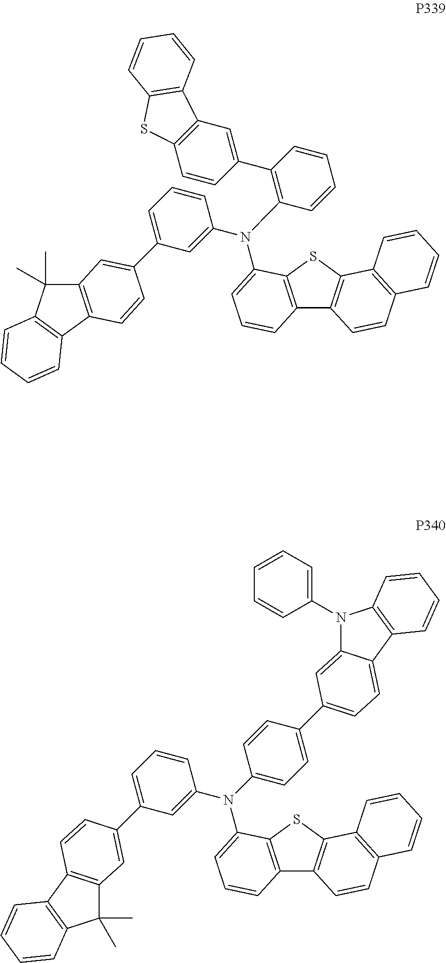 [SMILES: CC1(C)c2ccccc2-c2ccc(-c3cccc(N(c4ccccc4-c4ccc5sc6ccccc6c5c4)c4cccc5c4sc4c6ccccc6ccc54)c3)cc21 ; CC1(C)c2ccccc2-c2ccc(-c3cccc(N(c4ccc(-c5ccc6c7ccccc7n(-c7ccccc7)c6c5)cc4)c4cccc5c4sc4c6ccccc6ccc54)c3)cc21]

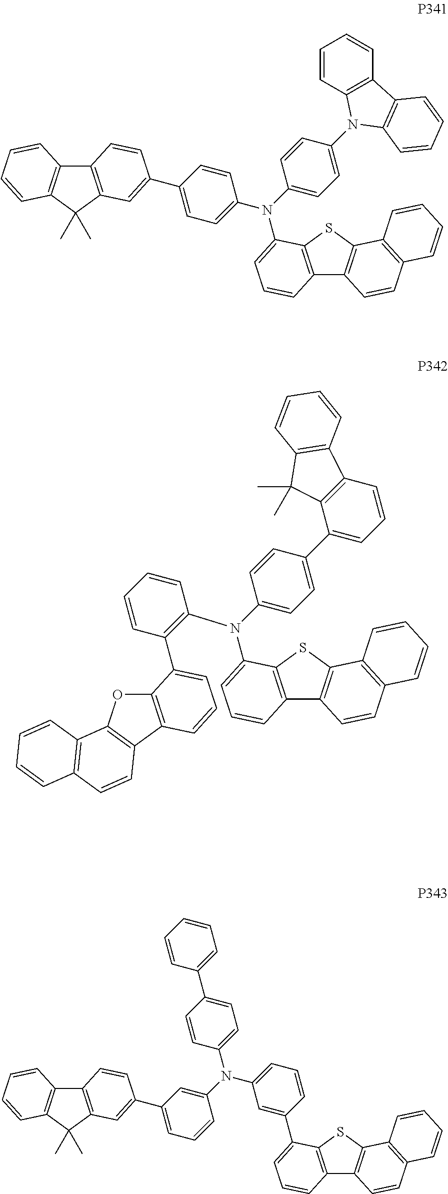 [SMILES: CC1(C)c2ccccc2-c2ccc(-c3ccc(N(c4ccc(-n5c6ccccc6c6ccccc65)cc4)c4cccc5c4sc4c6ccccc6ccc54)cc3)cc21 ; CC1(C)c2ccccc2-c2cccc(-c3ccc(N(c4ccccc4-c4cccc5c4oc4c6ccccc6ccc54)c4cccc5c4sc4c6ccccc6ccc54)cc3)c21 ; CC1(C)c2ccccc2-c2ccc(-c3cccc(N(c4ccc(-c5ccccc5)cc4)c4cccc(-c5cccc6c5sc5c7ccccc7ccc65)c4)c3)cc21]

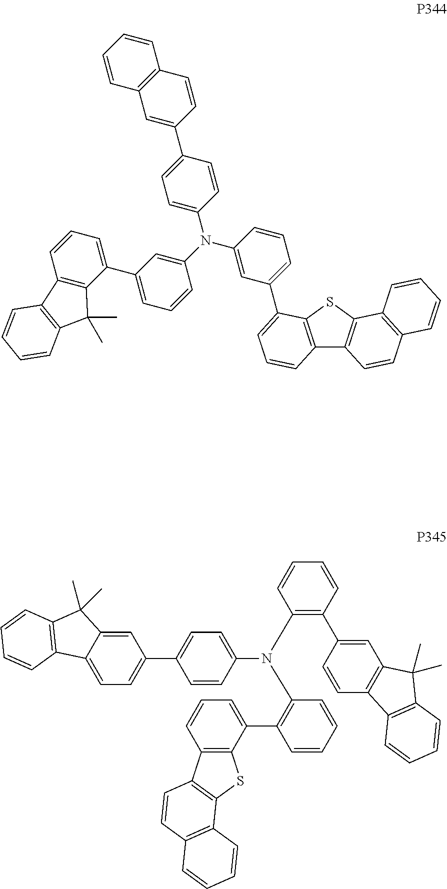 [SMILES: CC1(C)c2ccccc2-c2cccc(-c3cccc(N(c4ccc(-c5ccc6ccccc6c5)cc4)c4cccc(-c5cccc6c5sc5c7ccccc7ccc65)c4)c3)c21 ; CC1(C)c2ccccc2-c2ccc(-c3ccc(N(c4ccccc4-c4ccc5c(c4)C(C)(C)c4ccccc4-5)c4ccccc4-c4cccc5c4sc4c6ccccc6ccc54)cc3)cc21]

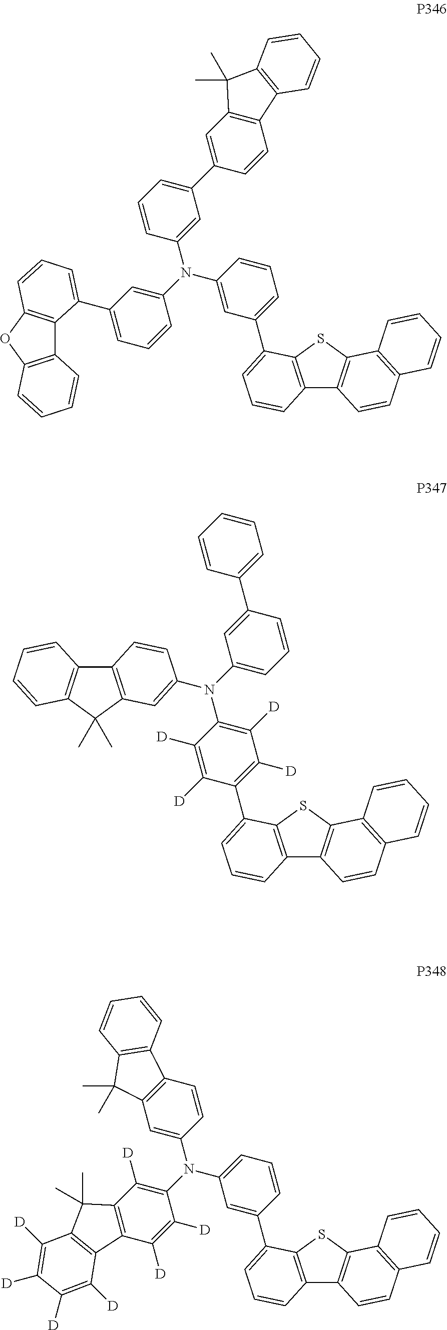 [SMILES: CC1(C)c2ccccc2-c2ccc(-c3cccc(N(c4cccc(-c5cccc6c5sc5c7ccccc7ccc65)c4)c4cccc(-c5cccc6oc7ccccc7c56)c4)c3)cc21 ; [2H]c1c([2H])c(N(c2cccc(-c3ccccc3)c2)c2ccc3c(c2)C(C)(C)c2ccccc2-3)c([2H])c([2H])c1-c1cccc2c1sc1c3ccccc3ccc21 ; [2H]c1c([2H])c([2H])c2c(c1[2H])-c1c([2H])c([2H])c(N(c3cccc(-c4cccc5c4sc4c6ccccc6ccc54)c3)c3ccc4c(c3)C(C)(C)c3ccccc3-4)c([2H])c1C2(C)C]

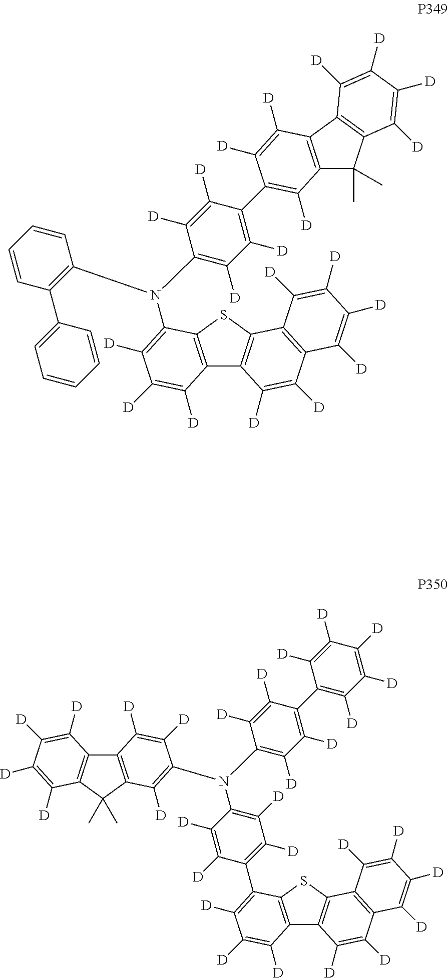 [SMILES: [2H]c1c([2H])c([2H])c2c(c1[2H])-c1c([2H])c([2H])c(-c3c([2H])c([2H])c(N(c4ccccc4-c4ccccc4)c4c([2H])c([2H])c([2H])c5c4sc4c6c([2H])c([2H])c([2H])c([2H])c6c([2H])c([2H])c45)c([2H])c3[2H])c([2H])c1C2(C)C ; [2H]c1c([2H])c([2H])c(-c2c([2H])c([2H])c(N(c3c([2H])c([2H])c(-c4c([2H])c([2H])c([2H])c5c4sc4c6c([2H])c([2H])c([2H])c([2H])c6c([2H])c([2H])c45)c([2H])c3[2H])c3c([2H])c([2H])c4c(c3[2H])C(C)(C)c3c([2H])c([2H])c([2H])c([2H])c3-4)c([2H])c2[2H])c([2H])c1[2H]]

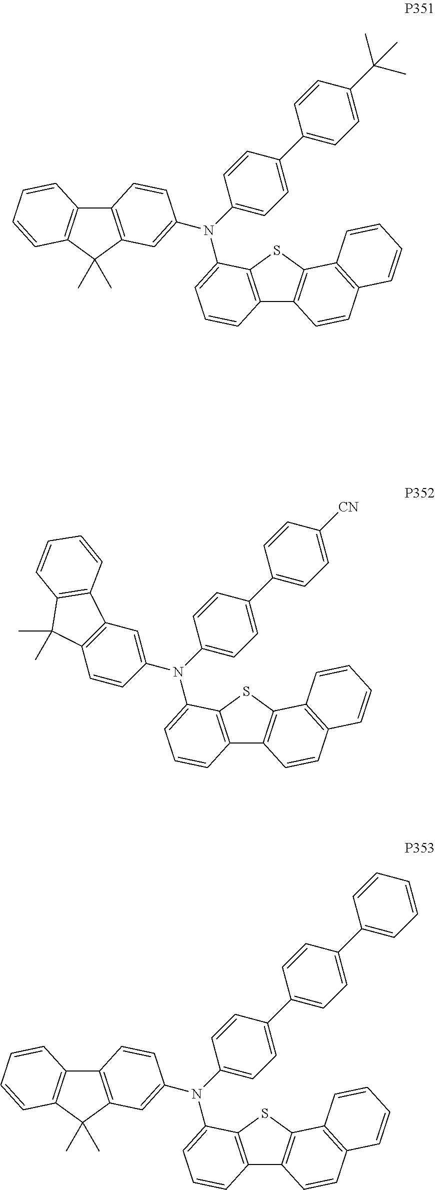

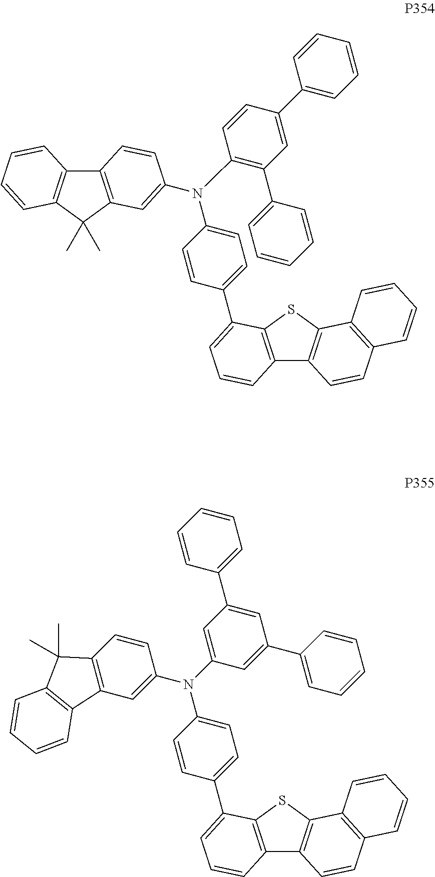 [SMILES: CC1(C)c2ccccc2-c2ccc(N(c3ccc(-c4cccc5c4sc4c6ccccc6ccc54)cc3)c3ccc(-c4ccccc4)cc3-c3ccccc3)cc21 ; CC1(C)c2ccccc2-c2cc(N(c3ccc(-c4cccc5c4sc4c6ccccc6ccc54)cc3)c3cc(-c4ccccc4)cc(-c4ccccc4)c3)ccc21]

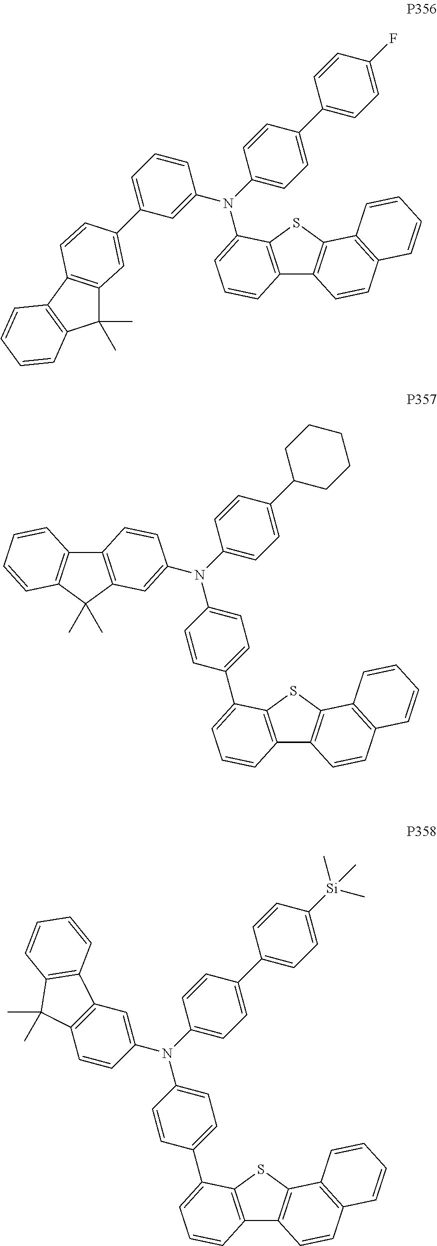 [SMILES: CC1(C)c2ccccc2-c2ccc(-c3cccc(N(c4ccc(-c5ccc(F)cc5)cc4)c4cccc5c4sc4c6ccccc6ccc54)c3)cc21 ; CC1(C)c2ccccc2-c2ccc(N(c3ccc(-c4cccc5c4sc4c6ccccc6ccc54)cc3)c3ccc(C4CCCCC4)cc3)cc21 ; CC1(C)c2ccccc2-c2cc(N(c3ccc(-c4ccc([Si](C)(C)C)cc4)cc3)c3ccc(-c4cccc5c4sc4c6ccccc6ccc54)cc3)ccc21]

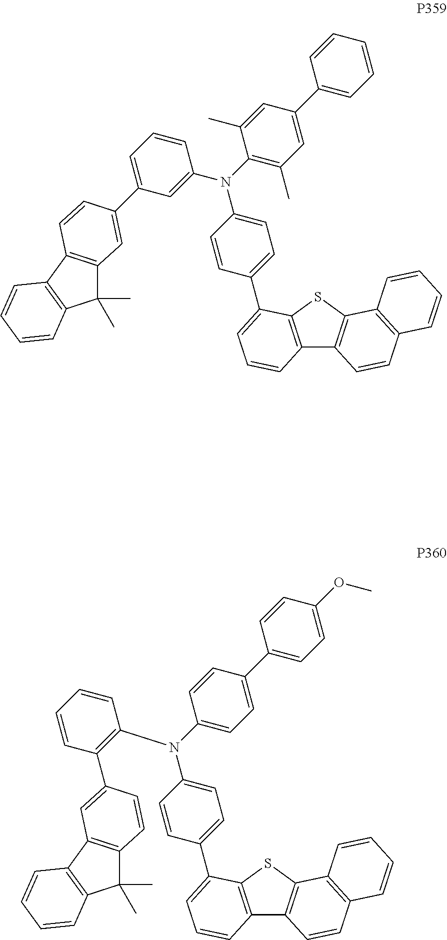 [SMILES: Cc1cc(-c2ccccc2)cc(C)c1N(c1ccc(-c2cccc3c2sc2c4ccccc4ccc32)cc1)c1cccc(-c2ccc3c(c2)C(C)(C)c2ccccc2-3)c1 ; COc1ccc(-c2ccc(N(c3ccc(-c4cccc5c4sc4c6ccccc6ccc54)cc3)c3ccccc3-c3ccc4c(c3)-c3ccccc3C4(C)C)cc2)cc1]

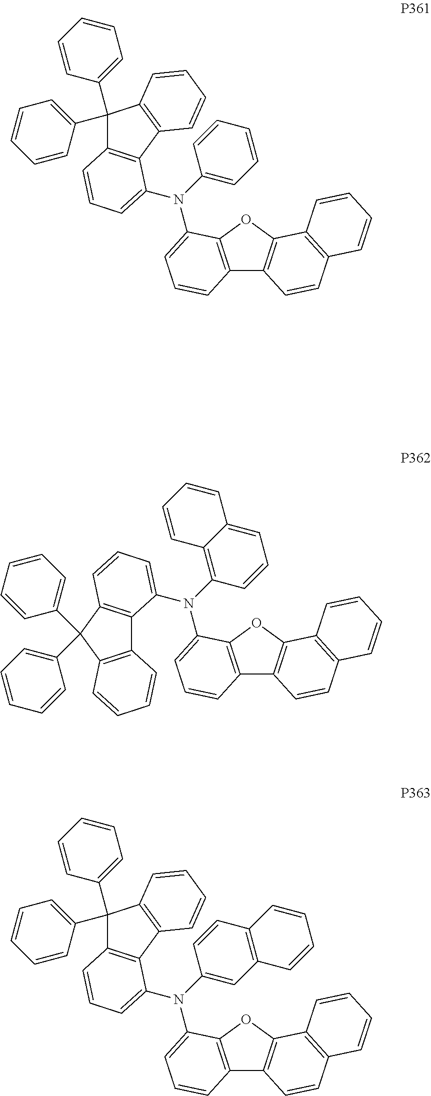 [SMILES: c1ccc(N(c2cccc3c2-c2ccccc2C3(c2ccccc2)c2ccccc2)c2cccc3c2oc2c4ccccc4ccc32)cc1 ; c1ccc(C2(c3ccccc3)c3ccccc3-c3c(N(c4cccc5ccccc45)c4cccc5c4oc4c6ccccc6ccc54)cccc32)cc1 ; c1ccc(C2(c3ccccc3)c3ccccc3-c3c(N(c4ccc5ccccc5c4)c4cccc5c4oc4c6ccccc6ccc54)cccc32)cc1]

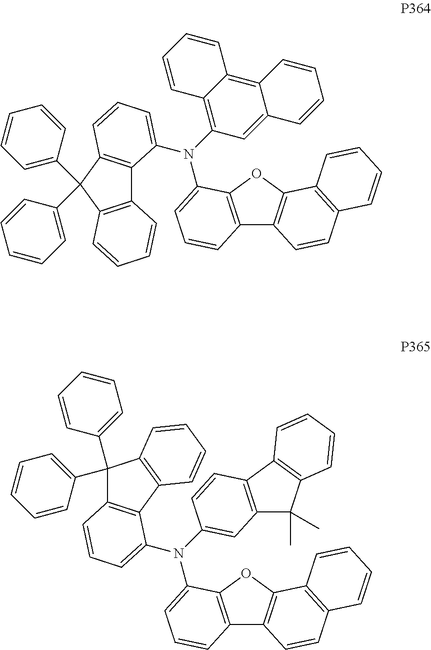 [SMILES: c1ccc(C2(c3ccccc3)c3ccccc3-c3c(N(c4cc5ccccc5c5ccccc45)c4cccc5c4oc4c6ccccc6ccc54)cccc32)cc1 ; CC1(C)c2ccccc2-c2ccc(N(c3cccc4c3-c3ccccc3C4(c3ccccc3)c3ccccc3)c3cccc4c3oc3c5ccccc5ccc43)cc21]

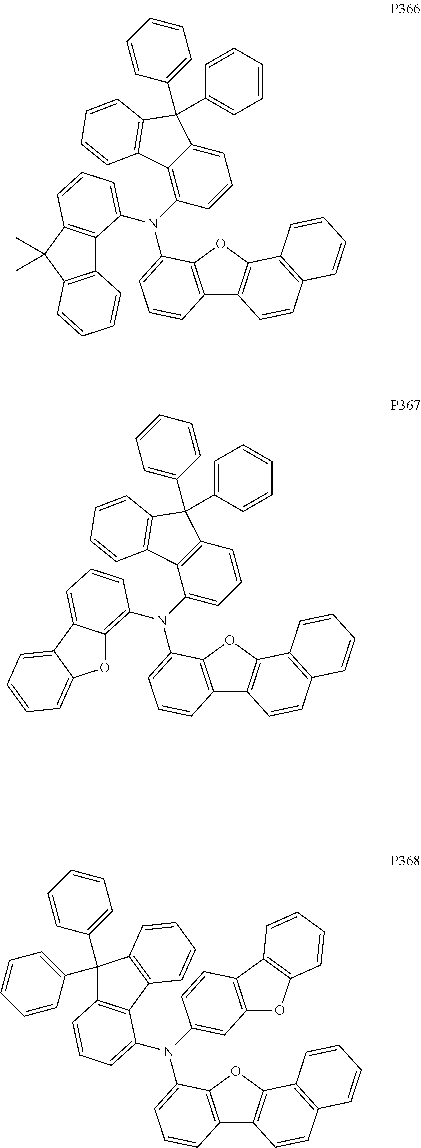 [SMILES: CC1(C)c2ccccc2-c2c(N(c3cccc4c3-c3ccccc3C4(c3ccccc3)c3ccccc3)c3cccc4c3oc3c5ccccc5ccc43)cccc21 ; c1ccc(C2(c3ccccc3)c3ccccc3-c3c(N(c4cccc5c4oc4ccccc45)c4cccc5c4oc4c6ccccc6ccc54)cccc32)cc1 ; c1ccc(C2(c3ccccc3)c3ccccc3-c3c(N(c4ccc5c(c4)oc4ccccc45)c4cccc5c4oc4c6ccccc6ccc54)cccc32)cc1]

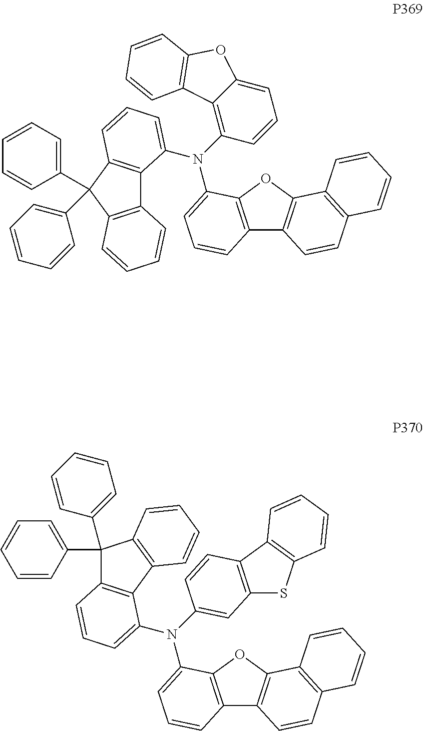 [SMILES: c1ccc(C2(c3ccccc3)c3ccccc3-c3c(N(c4cccc5c4oc4c6ccccc6ccc54)c4cccc5oc6ccccc6c45)cccc32)cc1 ; c1ccc(C2(c3ccccc3)c3ccccc3-c3c(N(c4ccc5c(c4)sc4ccccc45)c4cccc5c4oc4c6ccccc6ccc54)cccc32)cc1]

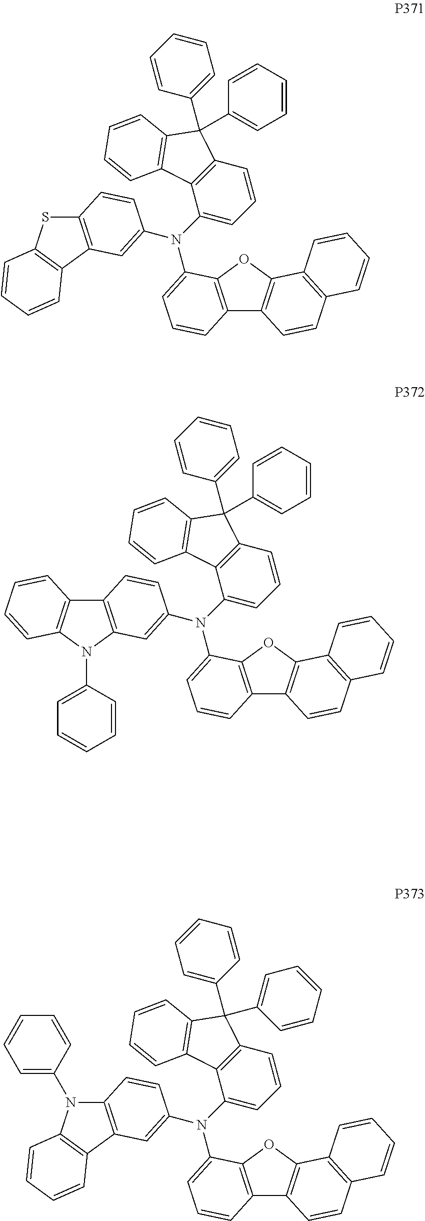 [SMILES: c1ccc(C2(c3ccccc3)c3ccccc3-c3c(N(c4ccc5sc6ccccc6c5c4)c4cccc5c4oc4c6ccccc6ccc54)cccc32)cc1 ; c1ccc(-n2c3ccccc3c3ccc(N(c4cccc5c4-c4ccccc4C5(c4ccccc4)c4ccccc4)c4cccc5c4oc4c6ccccc6ccc54)cc32)cc1 ; c1ccc(-n2c3ccccc3c3cc(N(c4cccc5c4-c4ccccc4C5(c4ccccc4)c4ccccc4)c4cccc5c4oc4c6ccccc6ccc54)ccc32)cc1]

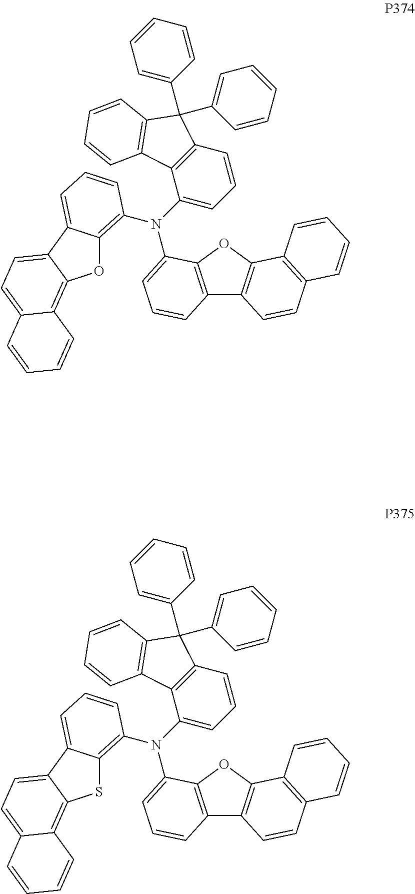 [SMILES: c1ccc(C2(c3ccccc3)c3ccccc3-c3c(N(c4cccc5c4oc4c6ccccc6ccc54)c4cccc5c4oc4c6ccccc6ccc54)cccc32)cc1 ; c1ccc(C2(c3ccccc3)c3ccccc3-c3c(N(c4cccc5c4oc4c6ccccc6ccc54)c4cccc5c4sc4c6ccccc6ccc54)cccc32)cc1]

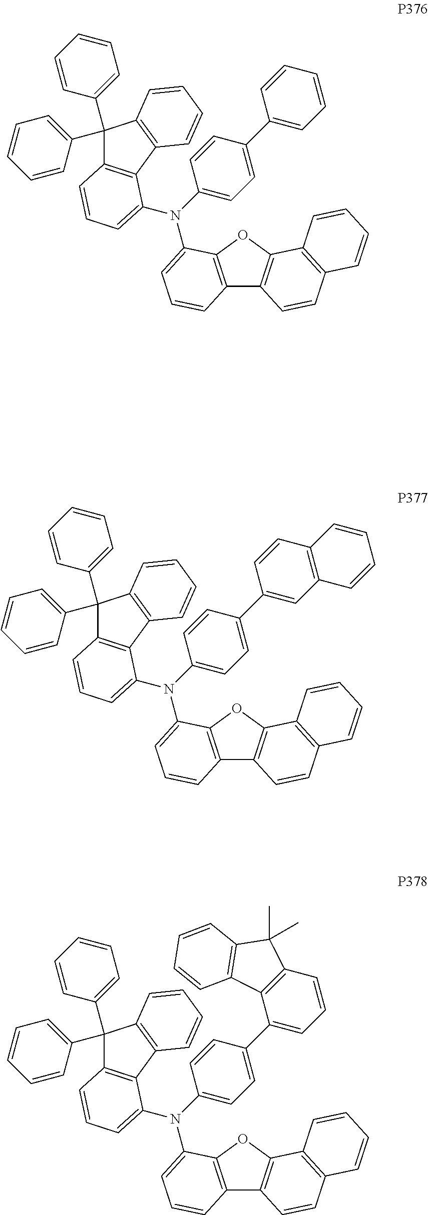 [SMILES: c1ccc(-c2ccc(N(c3cccc4c3-c3ccccc3C4(c3ccccc3)c3ccccc3)c3cccc4c3oc3c5ccccc5ccc43)cc2)cc1 ; c1ccc(C2(c3ccccc3)c3ccccc3-c3c(N(c4ccc(-c5ccc6ccccc6c5)cc4)c4cccc5c4oc4c6ccccc6ccc54)cccc32)cc1 ; CC1(C)c2ccccc2-c2c(-c3ccc(N(c4cccc5c4-c4ccccc4C5(c4ccccc4)c4ccccc4)c4cccc5c4oc4c6ccccc6ccc54)cc3)cccc21]

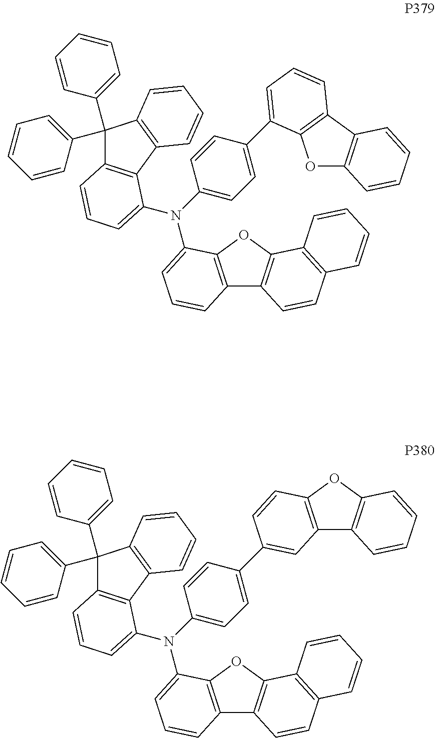 [SMILES: c1ccc(C2(c3ccccc3)c3ccccc3-c3c(N(c4ccc(-c5cccc6c5oc5ccccc56)cc4)c4cccc5c4oc4c6ccccc6ccc54)cccc32)cc1 ; c1ccc(C2(c3ccccc3)c3ccccc3-c3c(N(c4ccc(-c5ccc6oc7ccccc7c6c5)cc4)c4cccc5c4oc4c6ccccc6ccc54)cccc32)cc1]

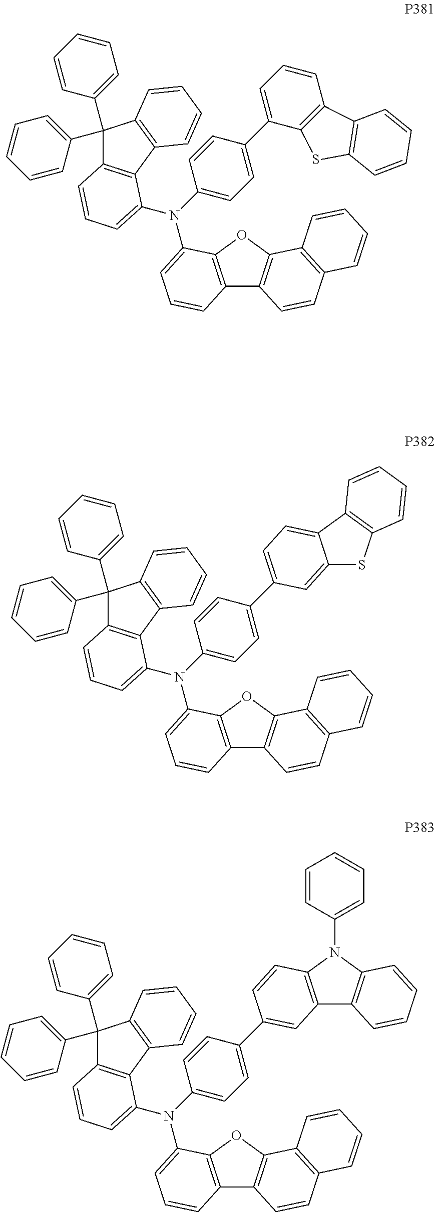 [SMILES: c1ccc(C2(c3ccccc3)c3ccccc3-c3c(N(c4ccc(-c5cccc6c5sc5ccccc56)cc4)c4cccc5c4oc4c6ccccc6ccc54)cccc32)cc1 ; c1ccc(C2(c3ccccc3)c3ccccc3-c3c(N(c4ccc(-c5ccc6c(c5)sc5ccccc56)cc4)c4cccc5c4oc4c6ccccc6ccc54)cccc32)cc1 ; c1ccc(-n2c3ccccc3c3cc(-c4ccc(N(c5cccc6c5-c5ccccc5C6(c5ccccc5)c5ccccc5)c5cccc6c5oc5c7ccccc7ccc65)cc4)ccc32)cc1]

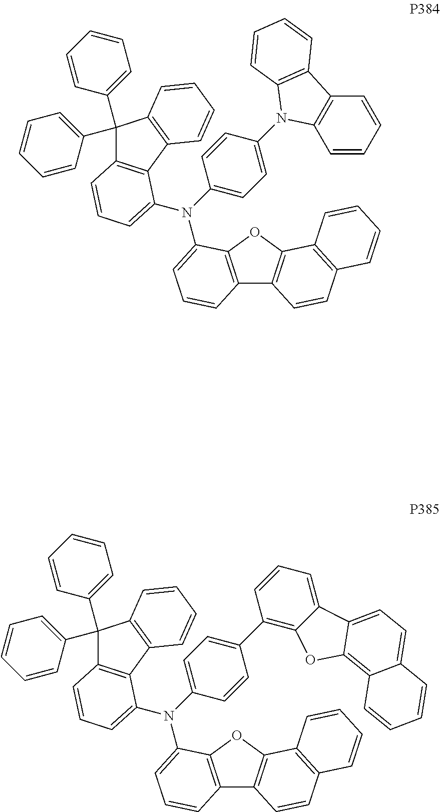 [SMILES: c1ccc(C2(c3ccccc3)c3ccccc3-c3c(N(c4ccc(-n5c6ccccc6c6ccccc65)cc4)c4cccc5c4oc4c6ccccc6ccc54)cccc32)cc1 ; c1ccc(C2(c3ccccc3)c3ccccc3-c3c(N(c4ccc(-c5cccc6c5oc5c7ccccc7ccc65)cc4)c4cccc5c4oc4c6ccccc6ccc54)cccc32)cc1]

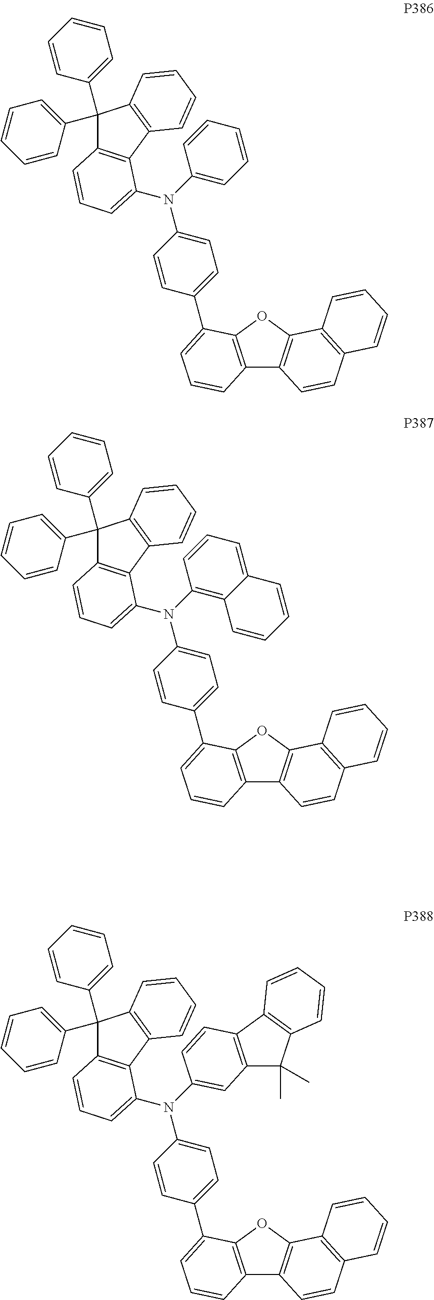 [SMILES: c1ccc(N(c2ccc(-c3cccc4c3oc3c5ccccc5ccc43)cc2)c2cccc3c2-c2ccccc2C3(c2ccccc2)c2ccccc2)cc1 ; c1ccc(C2(c3ccccc3)c3ccccc3-c3c(N(c4ccc(-c5cccc6c5oc5c7ccccc7ccc65)cc4)c4cccc5ccccc45)cccc32)cc1 ; CC1(C)c2ccccc2-c2ccc(N(c3ccc(-c4cccc5c4oc4c6ccccc6ccc54)cc3)c3cccc4c3-c3ccccc3C4(c3ccccc3)c3ccccc3)cc21]

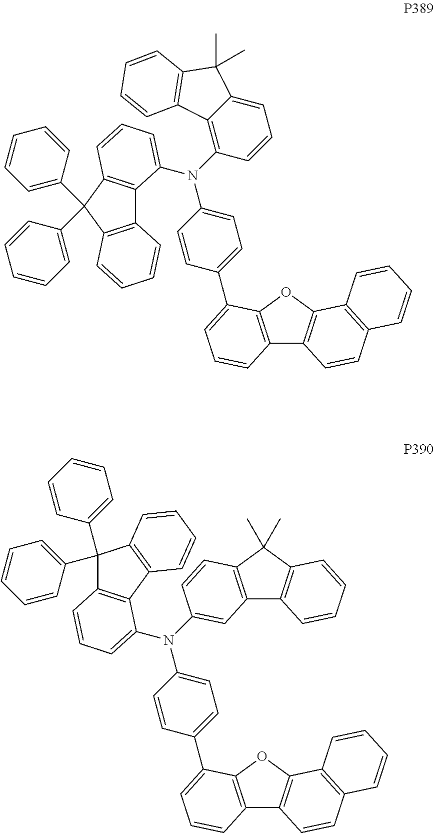 [SMILES: CC1(C)c2ccccc2-c2c(N(c3ccc(-c4cccc5c4oc4c6ccccc6ccc54)cc3)c3cccc4c3-c3ccccc3C4(c3ccccc3)c3ccccc3)cccc21 ; CC1(C)c2ccccc2-c2cc(N(c3ccc(-c4cccc5c4oc4c6ccccc6ccc54)cc3)c3cccc4c3-c3ccccc3C4(c3ccccc3)c3ccccc3)ccc21]

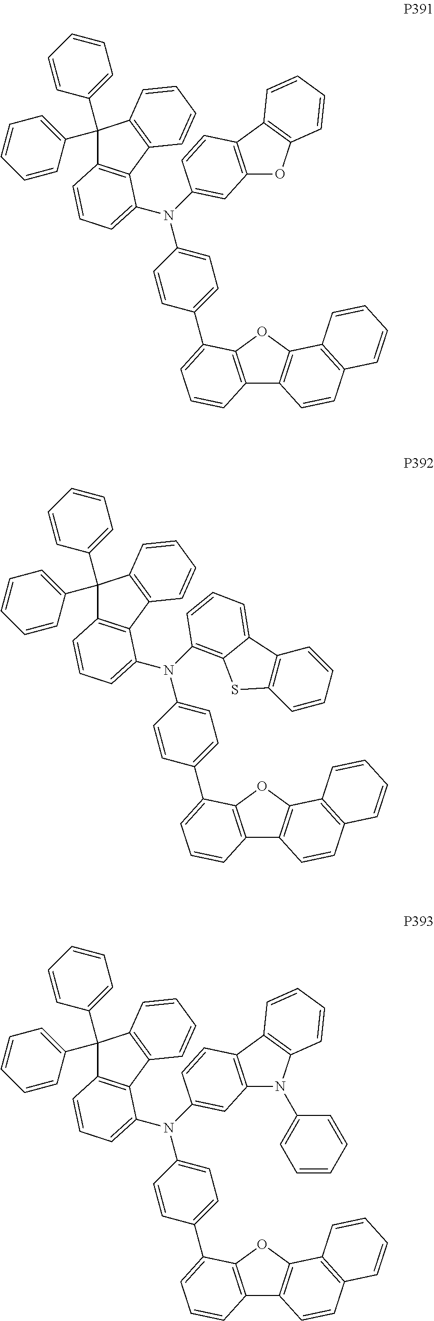 [SMILES: c1ccc(C2(c3ccccc3)c3ccccc3-c3c(N(c4ccc(-c5cccc6c5oc5c7ccccc7ccc65)cc4)c4ccc5c(c4)oc4ccccc45)cccc32)cc1 ; c1ccc(C2(c3ccccc3)c3ccccc3-c3c(N(c4ccc(-c5cccc6c5oc5c7ccccc7ccc65)cc4)c4cccc5c4sc4ccccc45)cccc32)cc1 ; c1ccc(-n2c3ccccc3c3ccc(N(c4ccc(-c5cccc6c5oc5c7ccccc7ccc65)cc4)c4cccc5c4-c4ccccc4C5(c4ccccc4)c4ccccc4)cc32)cc1]

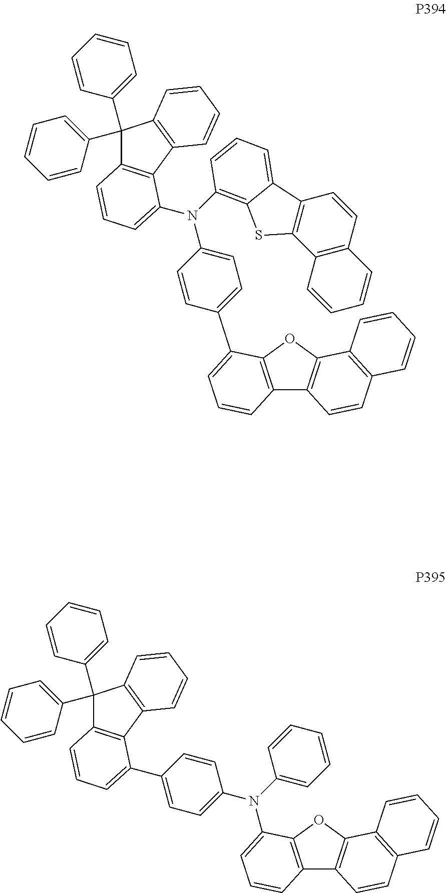 [SMILES: c1ccc(C2(c3ccccc3)c3ccccc3-c3c(N(c4ccc(-c5cccc6c5oc5c7ccccc7ccc65)cc4)c4cccc5c4sc4c6ccccc6ccc54)cccc32)cc1 ; c1ccc(N(c2ccc(-c3cccc4c3-c3ccccc3C4(c3ccccc3)c3ccccc3)cc2)c2cccc3c2oc2c4ccccc4ccc32)cc1]

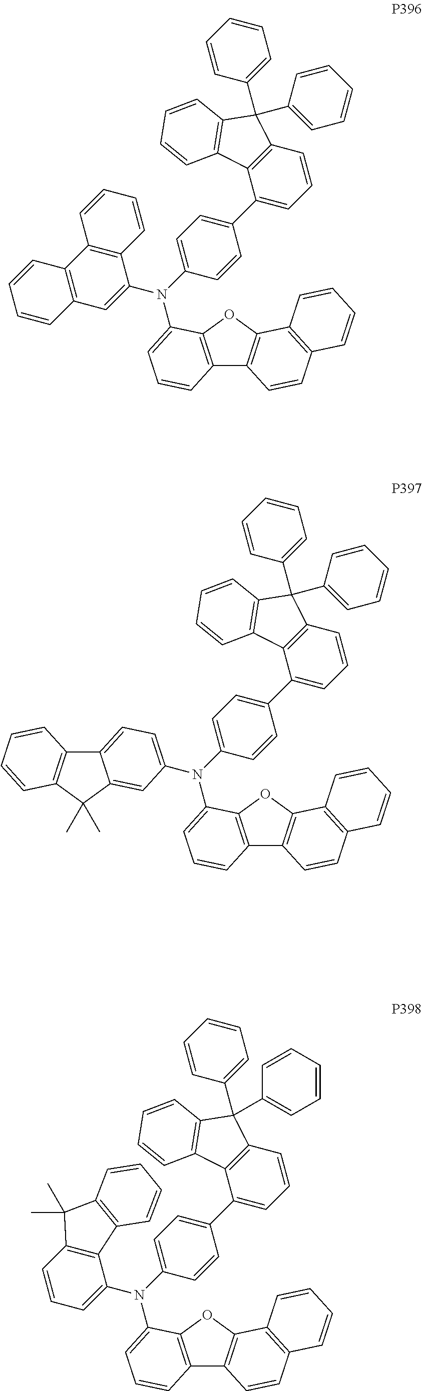 [SMILES: c1ccc(C2(c3ccccc3)c3ccccc3-c3c(-c4ccc(N(c5cc6ccccc6c6ccccc56)c5cccc6c5oc5c7ccccc7ccc65)cc4)cccc32)cc1 ; CC1(C)c2ccccc2-c2ccc(N(c3ccc(-c4cccc5c4-c4ccccc4C5(c4ccccc4)c4ccccc4)cc3)c3cccc4c3oc3c5ccccc5ccc43)cc21 ; CC1(C)c2ccccc2-c2c(N(c3ccc(-c4cccc5c4-c4ccccc4C5(c4ccccc4)c4ccccc4)cc3)c3cccc4c3oc3c5ccccc5ccc43)cccc21]

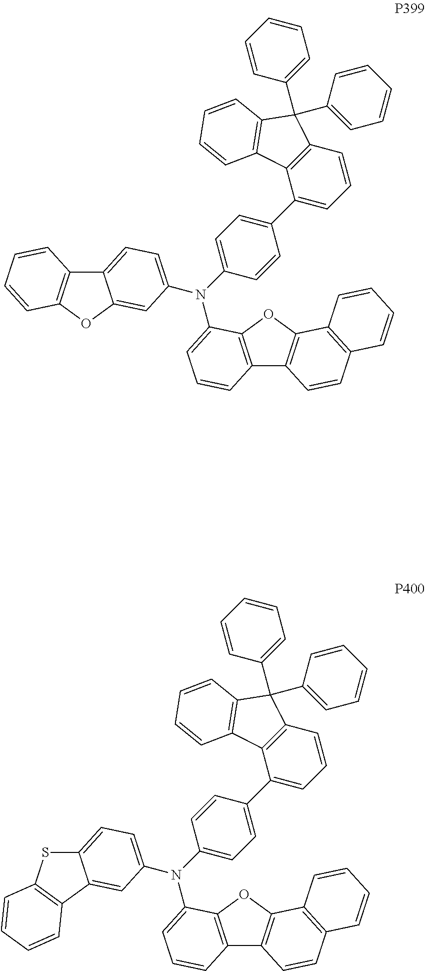 [SMILES: c1ccc(C2(c3ccccc3)c3ccccc3-c3c(-c4ccc(N(c5ccc6c(c5)oc5ccccc56)c5cccc6c5oc5c7ccccc7ccc65)cc4)cccc32)cc1 ; c1ccc(C2(c3ccccc3)c3ccccc3-c3c(-c4ccc(N(c5ccc6sc7ccccc7c6c5)c5cccc6c5oc5c7ccccc7ccc65)cc4)cccc32)cc1]

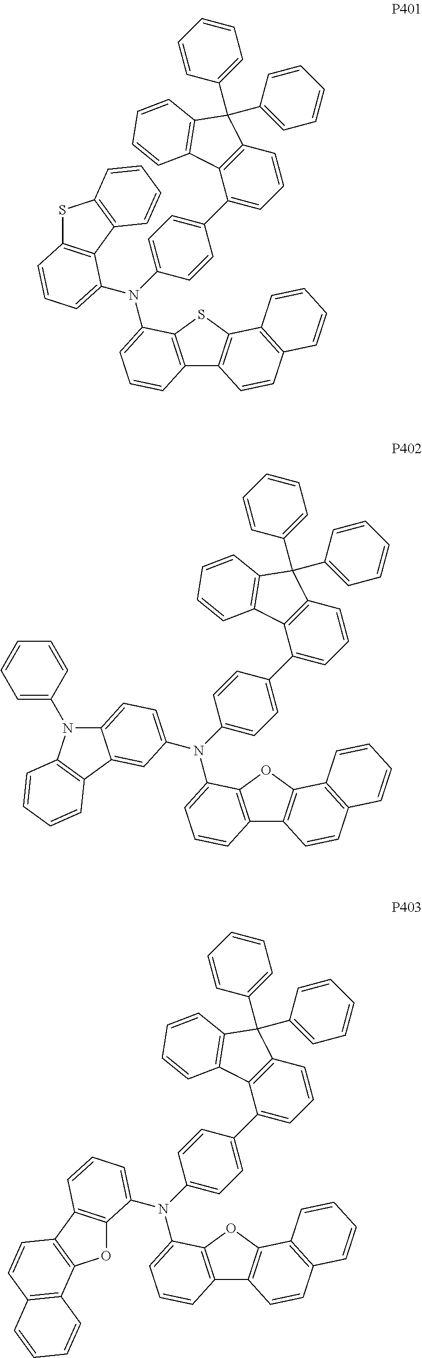 [SMILES: c1ccc(C2(c3ccccc3)c3ccccc3-c3c(-c4ccc(N(c5cccc6c5sc5c7ccccc7ccc65)c5cccc6sc7ccccc7c56)cc4)cccc32)cc1 ; c1ccc(-n2c3ccccc3c3cc(N(c4ccc(-c5cccc6c5-c5ccccc5C6(c5ccccc5)c5ccccc5)cc4)c4cccc5c4oc4c6ccccc6ccc54)ccc32)cc1 ; c1ccc(C2(c3ccccc3)c3ccccc3-c3c(-c4ccc(N(c5cccc6c5oc5c7ccccc7ccc65)c5cccc6c5oc5c7ccccc7ccc65)cc4)cccc32)cc1]

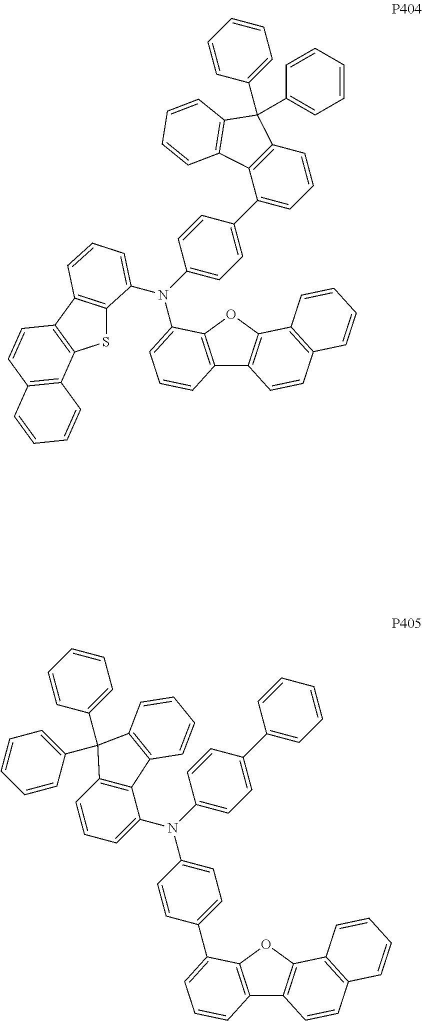 [SMILES: c1ccc(C2(c3ccccc3)c3ccccc3-c3c(-c4ccc(N(c5cccc6c5oc5c7ccccc7ccc65)c5cccc6c5sc5c7ccccc7ccc65)cc4)cccc32)cc1 ; c1ccc(-c2ccc(N(c3ccc(-c4cccc5c4oc4c6ccccc6ccc54)cc3)c3cccc4c3-c3ccccc3C4(c3ccccc3)c3ccccc3)cc2)cc1]

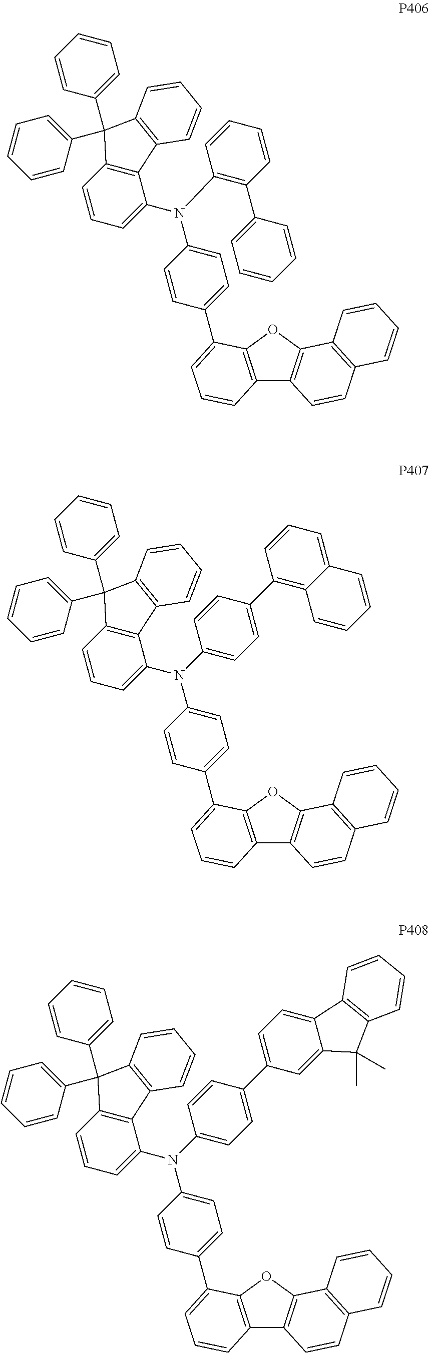 [SMILES: c1ccc(-c2ccccc2N(c2ccc(-c3cccc4c3oc3c5ccccc5ccc43)cc2)c2cccc3c2-c2ccccc2C3(c2ccccc2)c2ccccc2)cc1 ; c1ccc(C2(c3ccccc3)c3ccccc3-c3c(N(c4ccc(-c5cccc6ccccc56)cc4)c4ccc(-c5cccc6c5oc5c7ccccc7ccc65)cc4)cccc32)cc1 ; CC1(C)c2ccccc2-c2ccc(-c3ccc(N(c4ccc(-c5cccc6c5oc5c7ccccc7ccc65)cc4)c4cccc5c4-c4ccccc4C5(c4ccccc4)c4ccccc4)cc3)cc21]

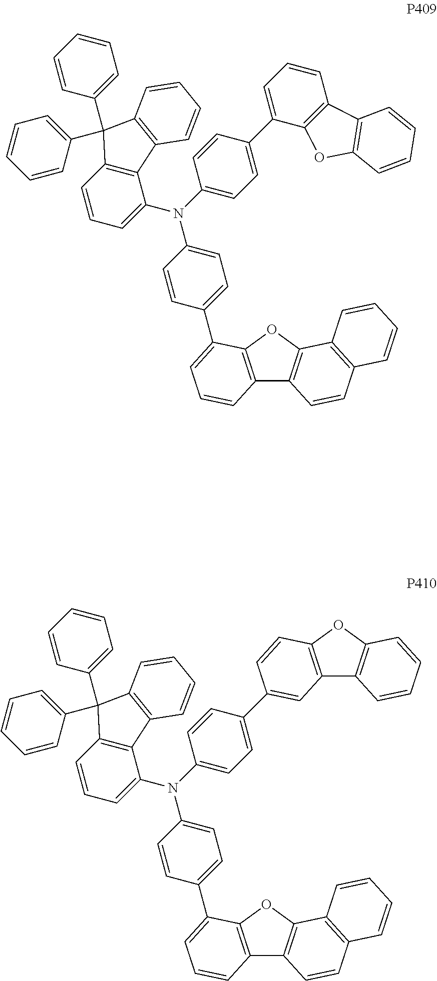 [SMILES: c1ccc(C2(c3ccccc3)c3ccccc3-c3c(N(c4ccc(-c5cccc6c5oc5ccccc56)cc4)c4ccc(-c5cccc6c5oc5c7ccccc7ccc65)cc4)cccc32)cc1 ; c1ccc(C2(c3ccccc3)c3ccccc3-c3c(N(c4ccc(-c5ccc6oc7ccccc7c6c5)cc4)c4ccc(-c5cccc6c5oc5c7ccccc7ccc65)cc4)cccc32)cc1]

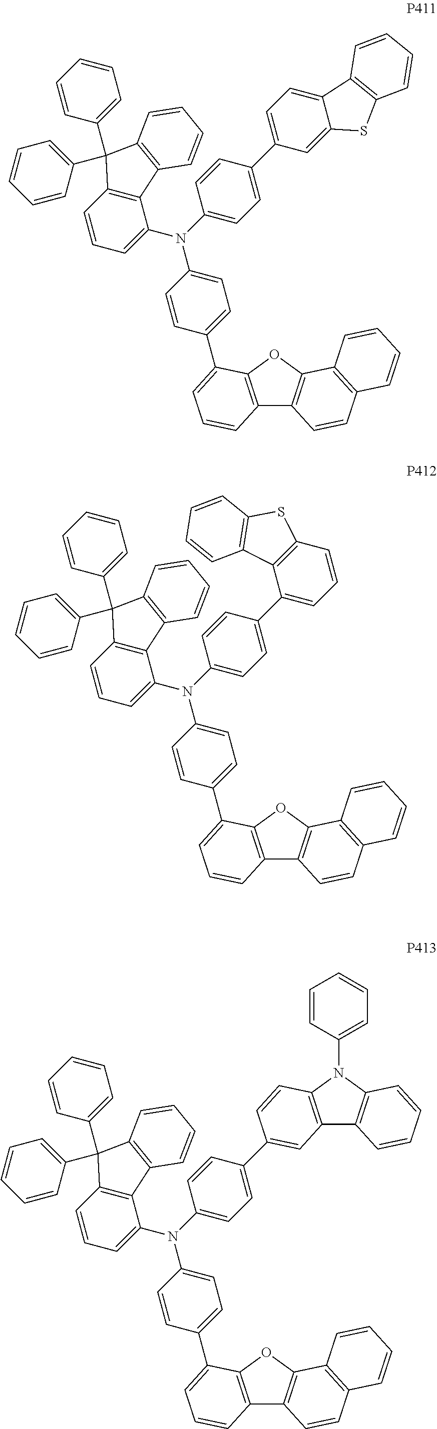 [SMILES: c1ccc(C2(c3ccccc3)c3ccccc3-c3c(N(c4ccc(-c5ccc6c(c5)sc5ccccc56)cc4)c4ccc(-c5cccc6c5oc5c7ccccc7ccc65)cc4)cccc32)cc1 ; c1ccc(C2(c3ccccc3)c3ccccc3-c3c(N(c4ccc(-c5cccc6c5oc5c7ccccc7ccc65)cc4)c4ccc(-c5cccc6sc7ccccc7c56)cc4)cccc32)cc1 ; c1ccc(-n2c3ccccc3c3cc(-c4ccc(N(c5ccc(-c6cccc7c6oc6c8ccccc8ccc76)cc5)c5cccc6c5-c5ccccc5C6(c5ccccc5)c5ccccc5)cc4)ccc32)cc1]

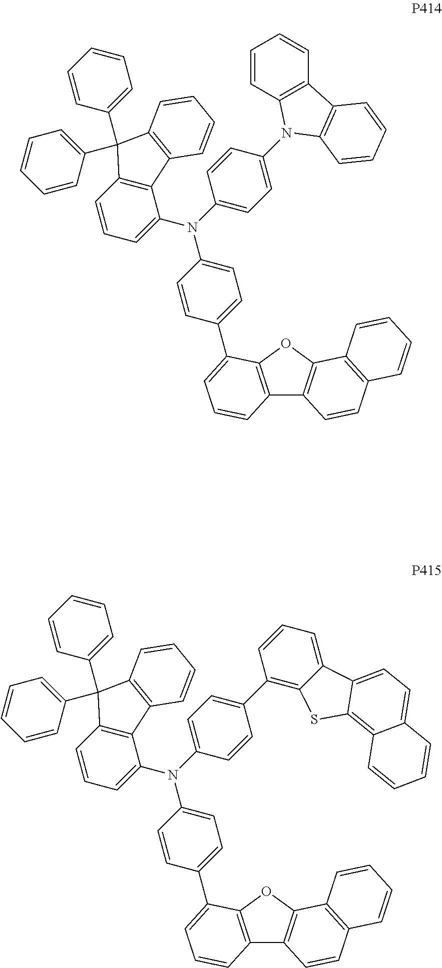 [SMILES: c1ccc(C2(c3ccccc3)c3ccccc3-c3c(N(c4ccc(-c5cccc6c5oc5c7ccccc7ccc65)cc4)c4ccc(-n5c6ccccc6c6ccccc65)cc4)cccc32)cc1 ; c1ccc(C2(c3ccccc3)c3ccccc3-c3c(N(c4ccc(-c5cccc6c5oc5c7ccccc7ccc65)cc4)c4ccc(-c5cccc6c5sc5c7ccccc7ccc65)cc4)cccc32)cc1]

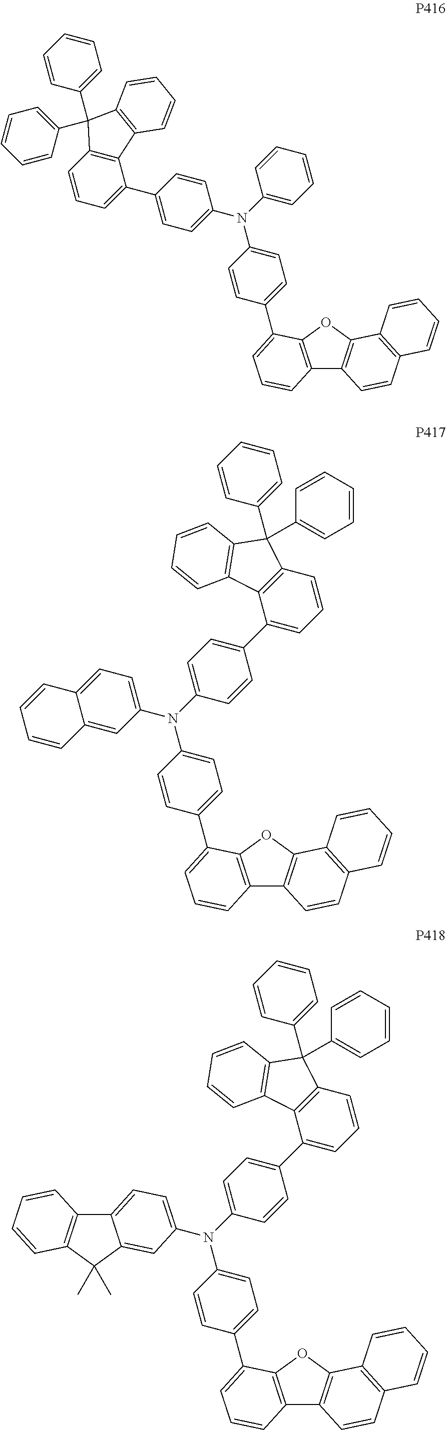 [SMILES: c1ccc(N(c2ccc(-c3cccc4c3-c3ccccc3C4(c3ccccc3)c3ccccc3)cc2)c2ccc(-c3cccc4c3oc3c5ccccc5ccc43)cc2)cc1 ; c1ccc(C2(c3ccccc3)c3ccccc3-c3c(-c4ccc(N(c5ccc(-c6cccc7c6oc6c8ccccc8ccc76)cc5)c5ccc6ccccc6c5)cc4)cccc32)cc1 ; CC1(C)c2ccccc2-c2ccc(N(c3ccc(-c4cccc5c4-c4ccccc4C5(c4ccccc4)c4ccccc4)cc3)c3ccc(-c4cccc5c4oc4c6ccccc6ccc54)cc3)cc21]

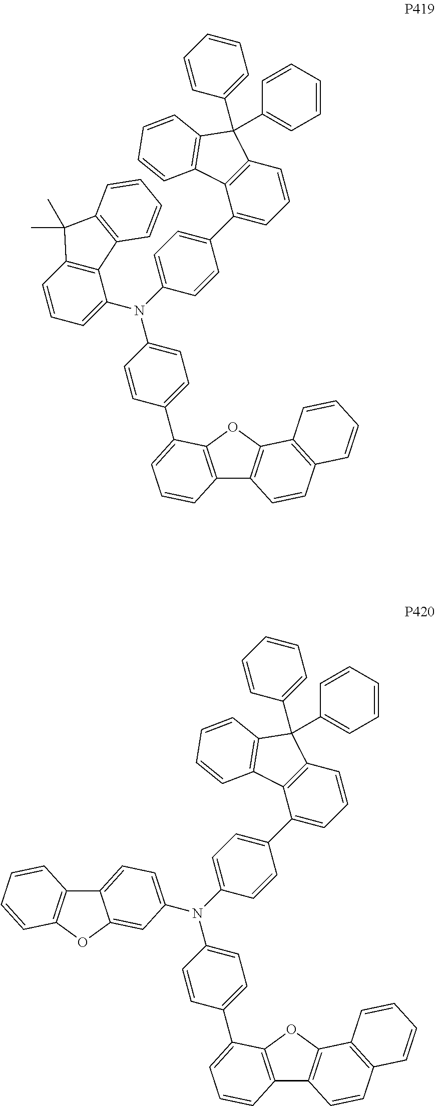 [SMILES: CC1(C)c2ccccc2-c2c(N(c3ccc(-c4cccc5c4-c4ccccc4C5(c4ccccc4)c4ccccc4)cc3)c3ccc(-c4cccc5c4oc4c6ccccc6ccc54)cc3)cccc21 ; c1ccc(C2(c3ccccc3)c3ccccc3-c3c(-c4ccc(N(c5ccc(-c6cccc7c6oc6c8ccccc8ccc76)cc5)c5ccc6c(c5)oc5ccccc56)cc4)cccc32)cc1]

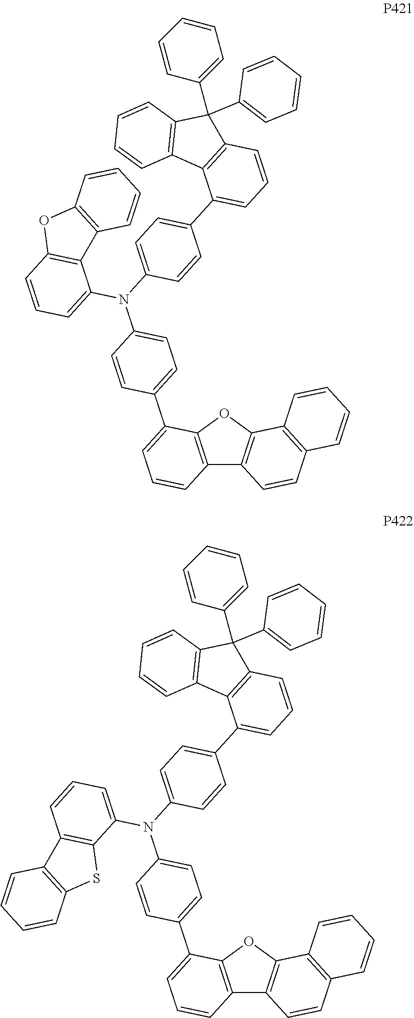 [SMILES: c1ccc(C2(c3ccccc3)c3ccccc3-c3c(-c4ccc(N(c5ccc(-c6cccc7c6oc6c8ccccc8ccc76)cc5)c5cccc6oc7ccccc7c56)cc4)cccc32)cc1 ; c1ccc(C2(c3ccccc3)c3ccccc3-c3c(-c4ccc(N(c5ccc(-c6cccc7c6oc6c8ccccc8ccc76)cc5)c5cccc6c5sc5ccccc56)cc4)cccc32)cc1]

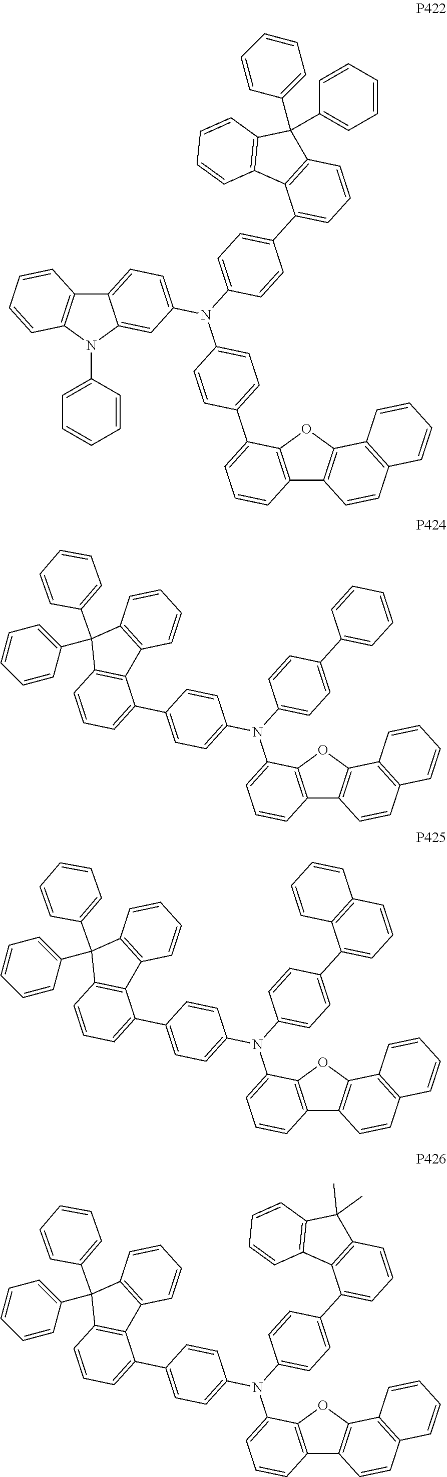 [SMILES: c1ccc(-n2c3ccccc3c3ccc(N(c4ccc(-c5cccc6c5-c5ccccc5C6(c5ccccc5)c5ccccc5)cc4)c4ccc(-c5cccc6c5oc5c7ccccc7ccc65)cc4)cc32)cc1 ; c1ccc(-c2ccc(N(c3ccc(-c4cccc5c4-c4ccccc4C5(c4ccccc4)c4ccccc4)cc3)c3cccc4c3oc3c5ccccc5ccc43)cc2)cc1 ; c1ccc(C2(c3ccccc3)c3ccccc3-c3c(-c4ccc(N(c5ccc(-c6cccc7ccccc67)cc5)c5cccc6c5oc5c7ccccc7ccc65)cc4)cccc32)cc1 ; CC1(C)c2ccccc2-c2c(-c3ccc(N(c4ccc(-c5cccc6c5-c5ccccc5C6(c5ccccc5)c5ccccc5)cc4)c4cccc5c4oc4c6ccccc6ccc54)cc3)cccc21]

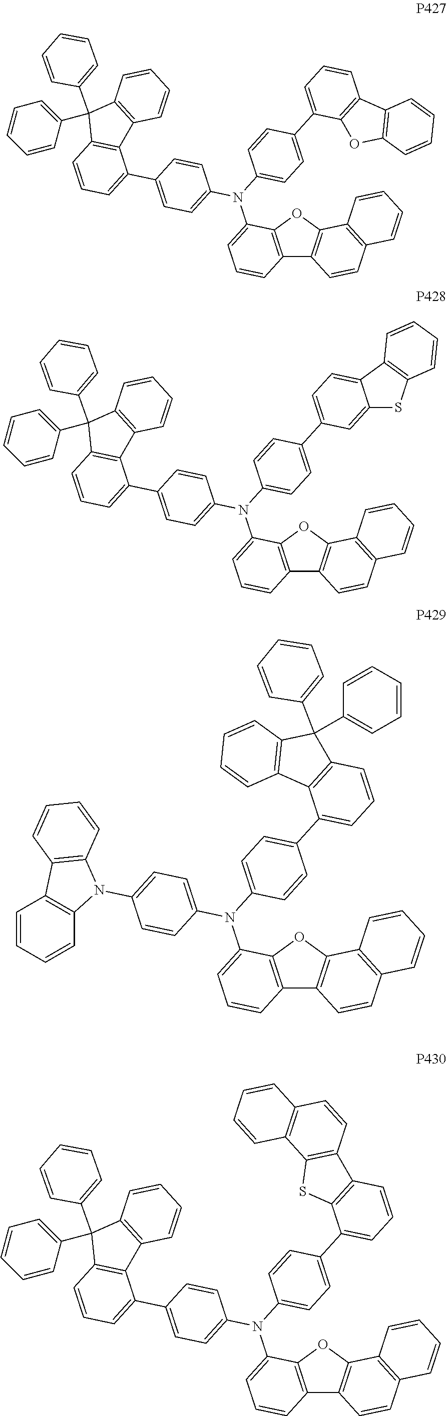 [SMILES: c1ccc(C2(c3ccccc3)c3ccccc3-c3c(-c4ccc(N(c5ccc(-c6cccc7c6oc6ccccc67)cc5)c5cccc6c5oc5c7ccccc7ccc65)cc4)cccc32)cc1 ; c1ccc(C2(c3ccccc3)c3ccccc3-c3c(-c4ccc(N(c5ccc(-c6ccc7c(c6)sc6ccccc67)cc5)c5cccc6c5oc5c7ccccc7ccc65)cc4)cccc32)cc1 ; c1ccc(C2(c3ccccc3)c3ccccc3-c3c(-c4ccc(N(c5ccc(-n6c7ccccc7c7ccccc76)cc5)c5cccc6c5oc5c7ccccc7ccc65)cc4)cccc32)cc1 ; c1ccc(C2(c3ccccc3)c3ccccc3-c3c(-c4ccc(N(c5ccc(-c6cccc7c6sc6c8ccccc8ccc76)cc5)c5cccc6c5oc5c7ccccc7ccc65)cc4)cccc32)cc1]

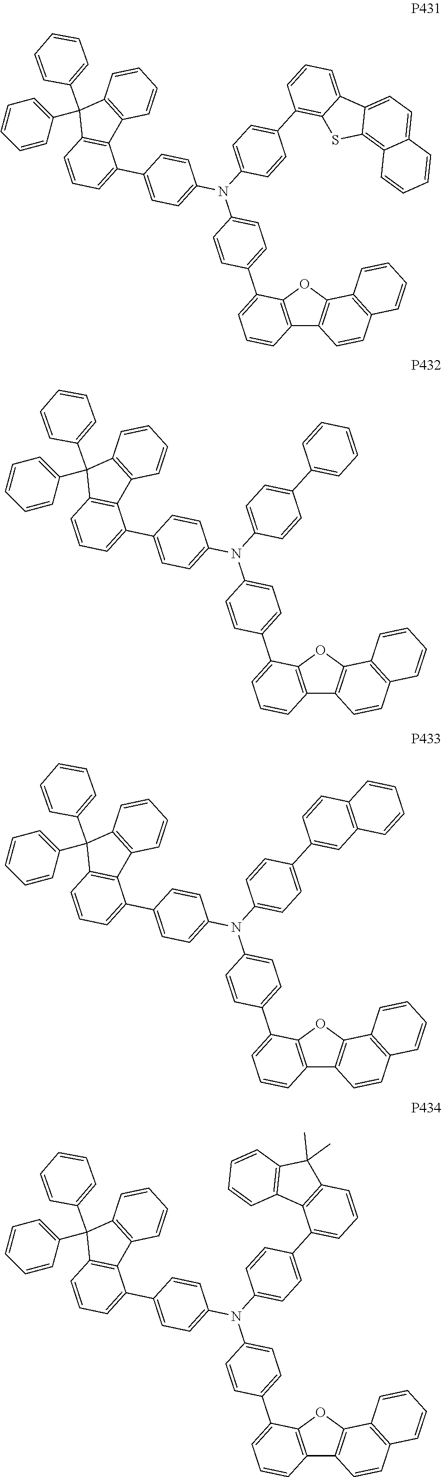 [SMILES: c1ccc(C2(c3ccccc3)c3ccccc3-c3c(-c4ccc(N(c5ccc(-c6cccc7c6oc6c8ccccc8ccc76)cc5)c5ccc(-c6cccc7c6sc6c8ccccc8ccc76)cc5)cc4)cccc32)cc1 ; c1ccc(-c2ccc(N(c3ccc(-c4cccc5c4-c4ccccc4C5(c4ccccc4)c4ccccc4)cc3)c3ccc(-c4cccc5c4oc4c6ccccc6ccc54)cc3)cc2)cc1 ; c1ccc(C2(c3ccccc3)c3ccccc3-c3c(-c4ccc(N(c5ccc(-c6ccc7ccccc7c6)cc5)c5ccc(-c6cccc7c6oc6c8ccccc8ccc76)cc5)cc4)cccc32)cc1 ; CC1(C)c2ccccc2-c2c(-c3ccc(N(c4ccc(-c5cccc6c5-c5ccccc5C6(c5ccccc5)c5ccccc5)cc4)c4ccc(-c5cccc6c5oc5c7ccccc7ccc65)cc4)cc3)cccc21]

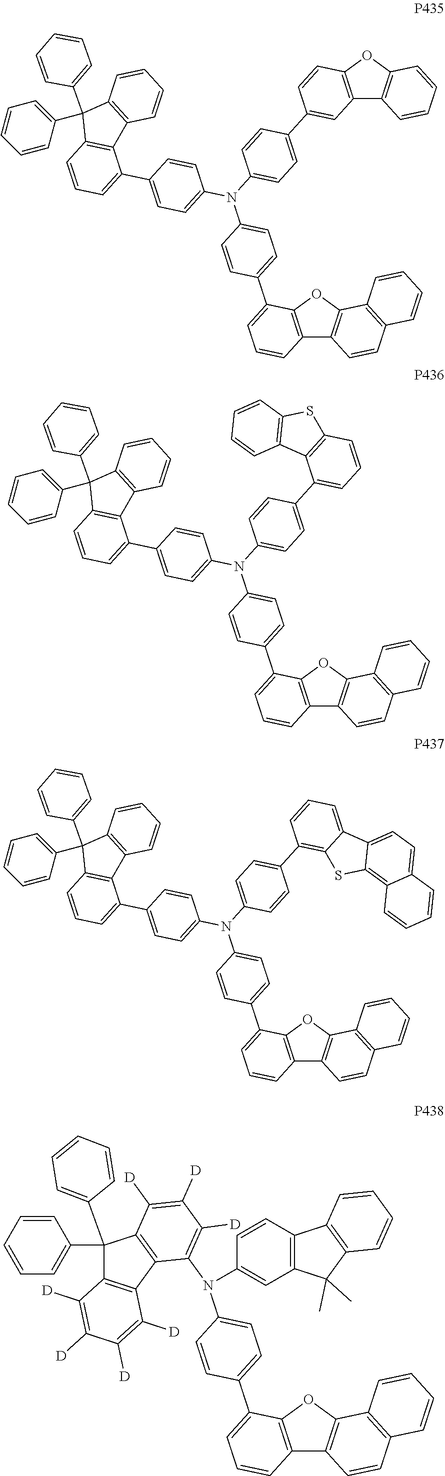

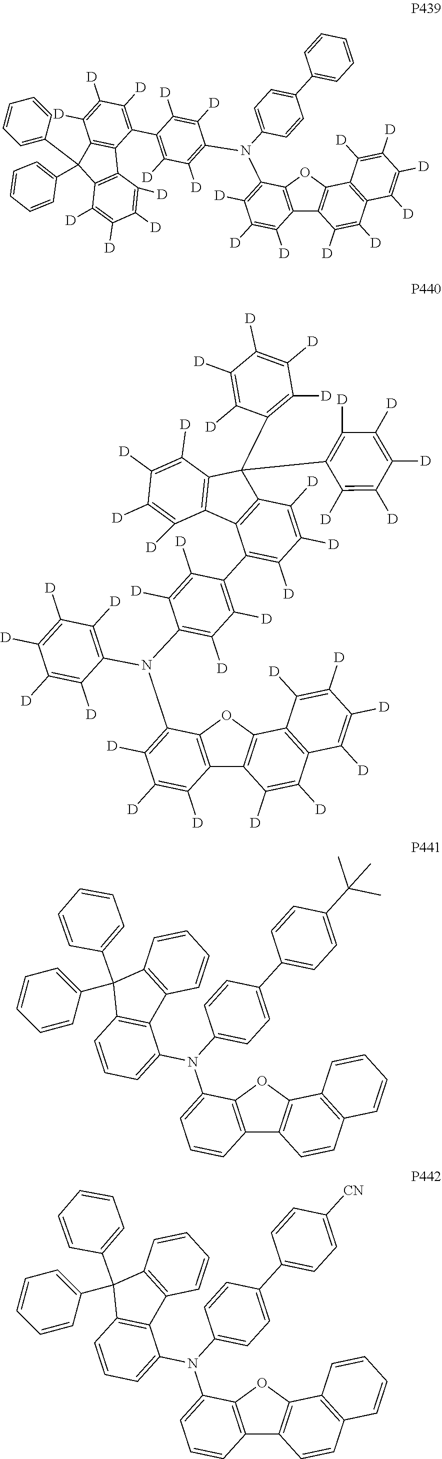

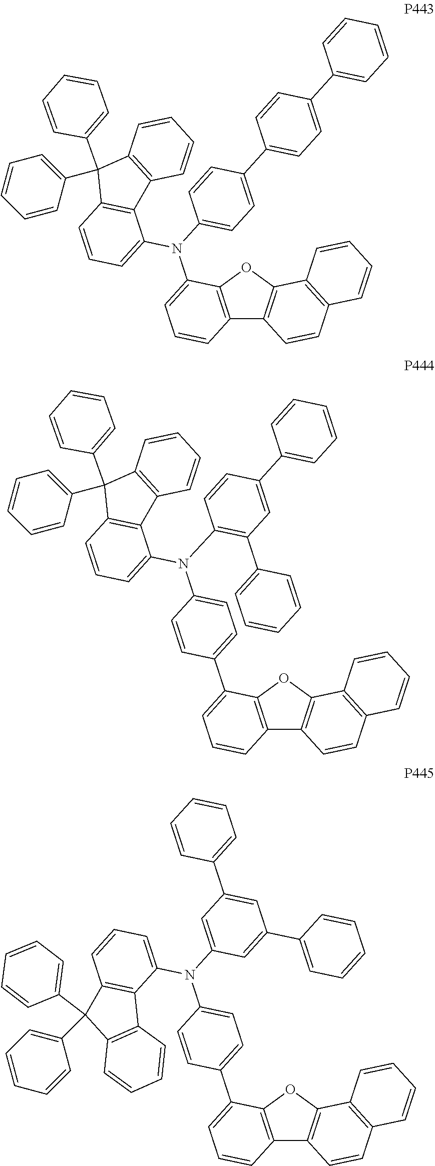 [SMILES: c1ccc(-c2ccc(-c3ccc(N(c4cccc5c4-c4ccccc4C5(c4ccccc4)c4ccccc4)c4cccc5c4oc4c6ccccc6ccc54)cc3)cc2)cc1 ; c1ccc(-c2ccc(N(c3ccc(-c4cccc5c4oc4c6ccccc6ccc54)cc3)c3cccc4c3-c3ccccc3C4(c3ccccc3)c3ccccc3)c(-c3ccccc3)c2)cc1 ; c1ccc(-c2cc(-c3ccccc3)cc(N(c3ccc(-c4cccc5c4oc4c6ccccc6ccc54)cc3)c3cccc4c3-c3ccccc3C4(c3ccccc3)c3ccccc3)c2)cc1]

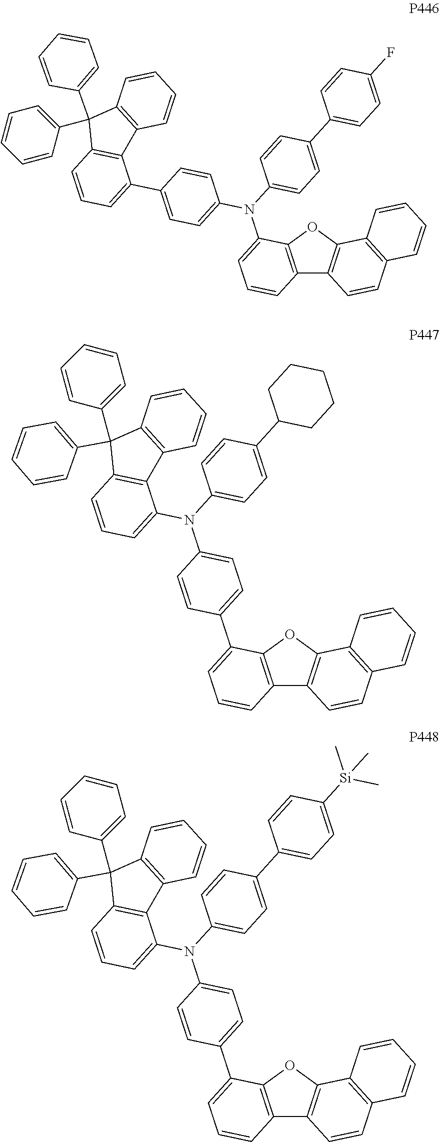

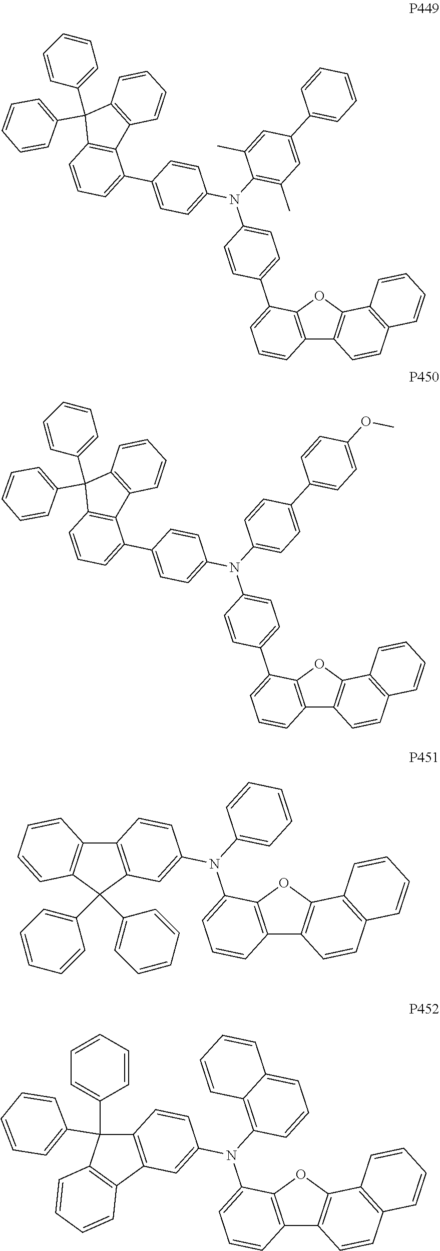 [SMILES: Cc1cc(-c2ccccc2)cc(C)c1N(c1ccc(-c2cccc3c2-c2ccccc2C3(c2ccccc2)c2ccccc2)cc1)c1ccc(-c2cccc3c2oc2c4ccccc4ccc32)cc1 ; COc1ccc(-c2ccc(N(c3ccc(-c4cccc5c4-c4ccccc4C5(c4ccccc4)c4ccccc4)cc3)c3ccc(-c4cccc5c4oc4c6ccccc6ccc54)cc3)cc2)cc1 ; c1ccc(N(c2ccc3c(c2)C(c2ccccc2)(c2ccccc2)c2ccccc2-3)c2cccc3c2oc2c4ccccc4ccc32)cc1 ; c1ccc(C2(c3ccccc3)c3ccccc3-c3cc(N(c4cccc5ccccc45)c4cccc5c4oc4c6ccccc6ccc54)ccc32)cc1]

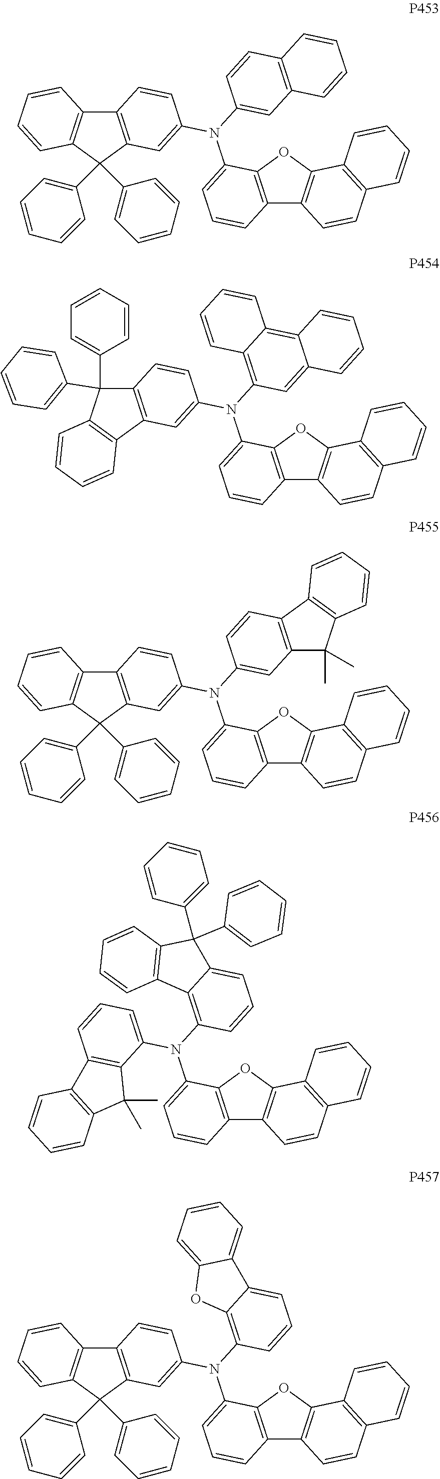 [SMILES: c1ccc(C2(c3ccccc3)c3ccccc3-c3ccc(N(c4ccc5ccccc5c4)c4cccc5c4oc4c6ccccc6ccc54)cc32)cc1 ; c1ccc(C2(c3ccccc3)c3ccccc3-c3cc(N(c4cc5ccccc5c5ccccc45)c4cccc5c4oc4c6ccccc6ccc54)ccc32)cc1 ; CC1(C)c2ccccc2-c2ccc(N(c3ccc4c(c3)C(c3ccccc3)(c3ccccc3)c3ccccc3-4)c3cccc4c3oc3c5ccccc5ccc43)cc21 ; CC1(C)c2ccccc2-c2cccc(N(c3cccc4c3-c3ccccc3C4(c3ccccc3)c3ccccc3)c3cccc4c3oc3c5ccccc5ccc43)c21 ; c1ccc(C2(c3ccccc3)c3ccccc3-c3ccc(N(c4cccc5c4oc4ccccc45)c4cccc5c4oc4c6ccccc6ccc54)cc32)cc1]

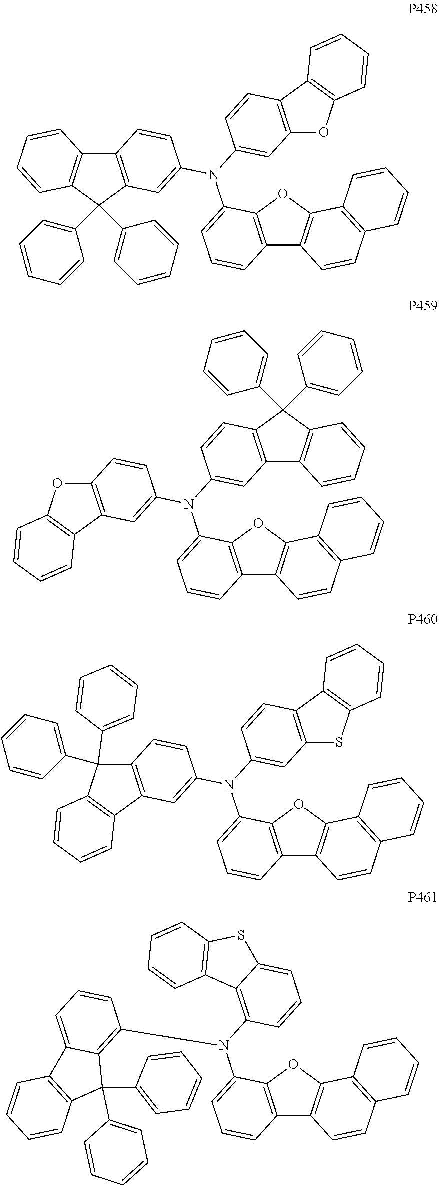 [SMILES: c1ccc(C2(c3ccccc3)c3ccccc3-c3ccc(N(c4ccc5c(c4)oc4ccccc45)c4cccc5c4oc4c6ccccc6ccc54)cc32)cc1 ; c1ccc(C2(c3ccccc3)c3ccccc3-c3cc(N(c4ccc5oc6ccccc6c5c4)c4cccc5c4oc4c6ccccc6ccc54)ccc32)cc1 ; c1ccc(C2(c3ccccc3)c3ccccc3-c3cc(N(c4ccc5c(c4)sc4ccccc45)c4cccc5c4oc4c6ccccc6ccc54)ccc32)cc1 ; c1ccc(C2(c3ccccc3)c3ccccc3-c3cccc(N(c4cccc5c4oc4c6ccccc6ccc54)c4cccc5sc6ccccc6c45)c32)cc1]

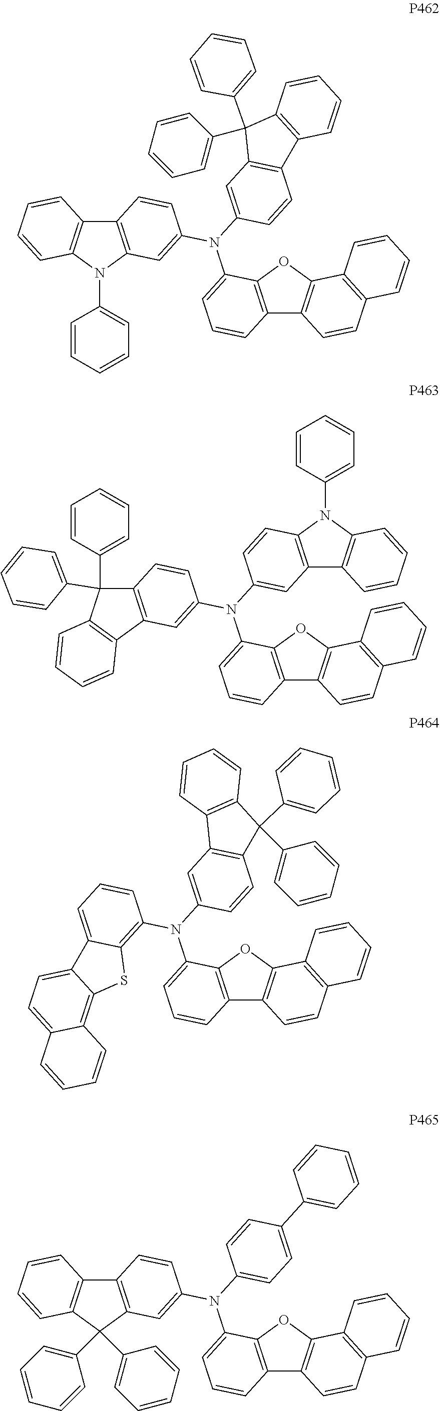 [SMILES: c1ccc(-n2c3ccccc3c3ccc(N(c4ccc5c(c4)C(c4ccccc4)(c4ccccc4)c4ccccc4-5)c4cccc5c4oc4c6ccccc6ccc54)cc32)cc1 ; c1ccc(-n2c3ccccc3c3cc(N(c4ccc5c(c4)-c4ccccc4C5(c4ccccc4)c4ccccc4)c4cccc5c4oc4c6ccccc6ccc54)ccc32)cc1 ; c1ccc(C2(c3ccccc3)c3ccccc3-c3cc(N(c4cccc5c4oc4c6ccccc6ccc54)c4cccc5c4sc4c6ccccc6ccc54)ccc32)cc1 ; c1ccc(-c2ccc(N(c3ccc4c(c3)C(c3ccccc3)(c3ccccc3)c3ccccc3-4)c3cccc4c3oc3c5ccccc5ccc43)cc2)cc1]

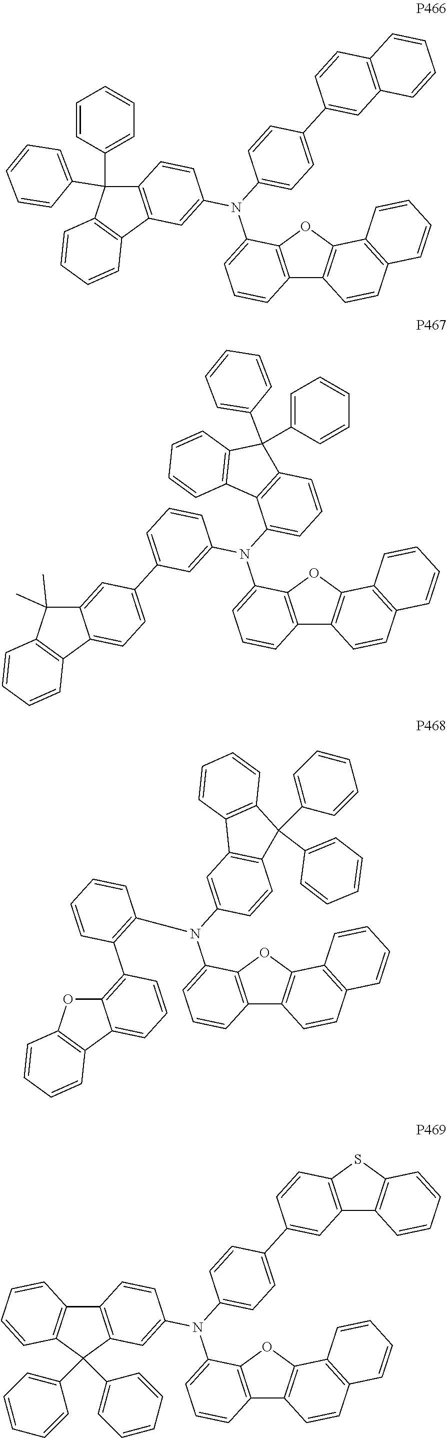 [SMILES: c1ccc(C2(c3ccccc3)c3ccccc3-c3cc(N(c4ccc(-c5ccc6ccccc6c5)cc4)c4cccc5c4oc4c6ccccc6ccc54)ccc32)cc1 ; CC1(C)c2ccccc2-c2ccc(-c3cccc(N(c4cccc5c4-c4ccccc4C5(c4ccccc4)c4ccccc4)c4cccc5c4oc4c6ccccc6ccc54)c3)cc21 ; c1ccc(C2(c3ccccc3)c3ccccc3-c3cc(N(c4ccccc4-c4cccc5c4oc4ccccc45)c4cccc5c4oc4c6ccccc6ccc54)ccc32)cc1 ; c1ccc(C2(c3ccccc3)c3ccccc3-c3ccc(N(c4ccc(-c5ccc6sc7ccccc7c6c5)cc4)c4cccc5c4oc4c6ccccc6ccc54)cc32)cc1]

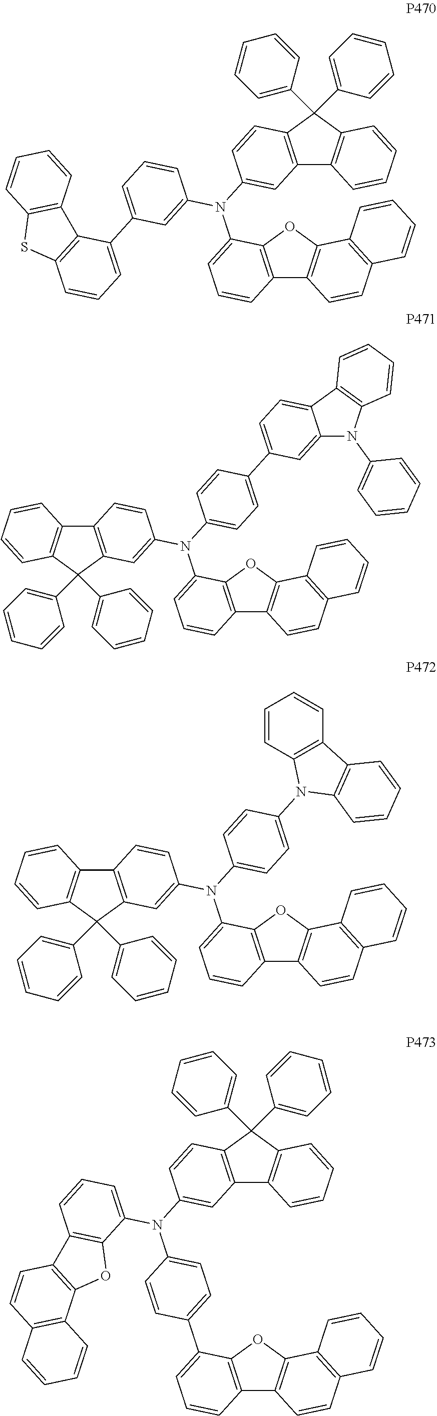 [SMILES: c1ccc(C2(c3ccccc3)c3ccccc3-c3cc(N(c4cccc(-c5cccc6sc7ccccc7c56)c4)c4cccc5c4oc4c6ccccc6ccc54)ccc32)cc1 ; c1ccc(-n2c3ccccc3c3ccc(-c4ccc(N(c5ccc6c(c5)C(c5ccccc5)(c5ccccc5)c5ccccc5-6)c5cccc6c5oc5c7ccccc7ccc65)cc4)cc32)cc1 ; c1ccc(C2(c3ccccc3)c3ccccc3-c3ccc(N(c4ccc(-n5c6ccccc6c6ccccc65)cc4)c4cccc5c4oc4c6ccccc6ccc54)cc32)cc1 ; c1ccc(C2(c3ccccc3)c3ccccc3-c3cc(N(c4ccc(-c5cccc6c5oc5c7ccccc7ccc65)cc4)c4cccc5c4oc4c6ccccc6ccc54)ccc32)cc1]

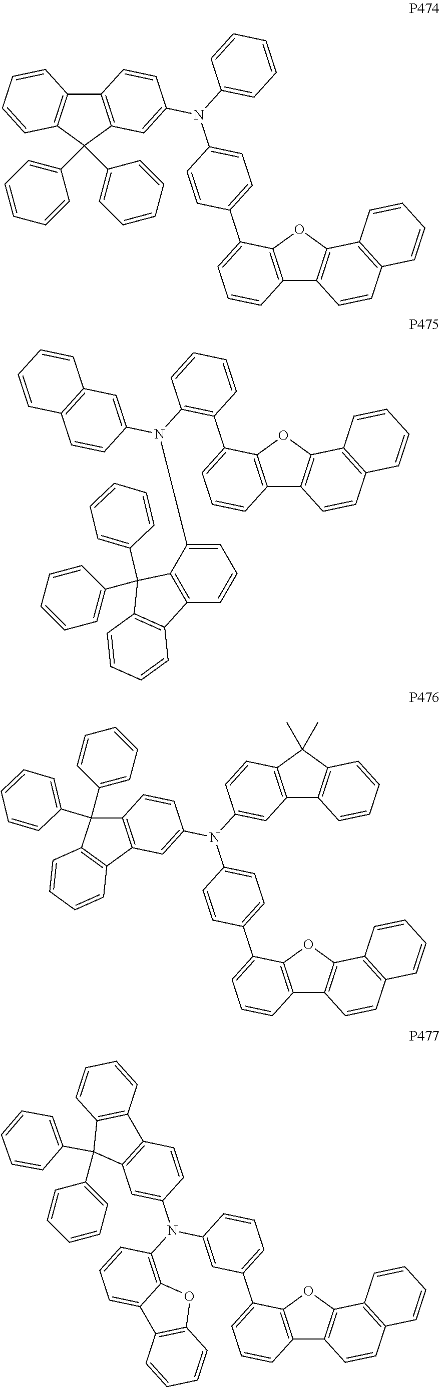 [SMILES: c1ccc(N(c2ccc(-c3cccc4c3oc3c5ccccc5ccc43)cc2)c2ccc3c(c2)C(c2ccccc2)(c2ccccc2)c2ccccc2-3)cc1 ; c1ccc(C2(c3ccccc3)c3ccccc3-c3cccc(N(c4ccc5ccccc5c4)c4ccccc4-c4cccc5c4oc4c6ccccc6ccc54)c32)cc1 ; CC1(C)c2ccccc2-c2cc(N(c3ccc(-c4cccc5c4oc4c6ccccc6ccc54)cc3)c3ccc4c(c3)-c3ccccc3C4(c3ccccc3)c3ccccc3)ccc21 ; c1ccc(C2(c3ccccc3)c3ccccc3-c3ccc(N(c4cccc(-c5cccc6c5oc5c7ccccc7ccc65)c4)c4cccc5c4oc4ccccc45)cc32)cc1]

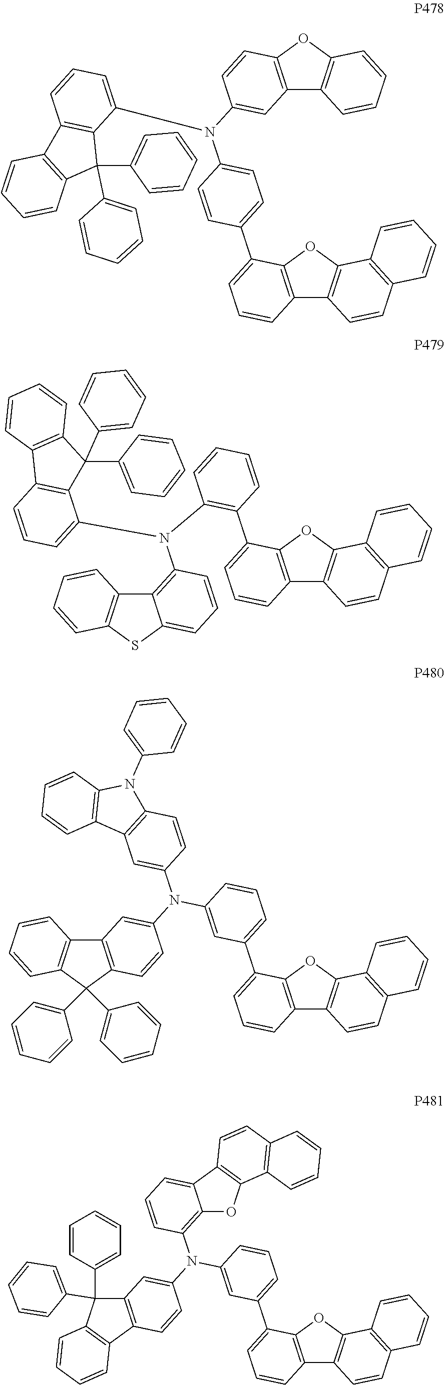 [SMILES: c1ccc(C2(c3ccccc3)c3ccccc3-c3cccc(N(c4ccc(-c5cccc6c5oc5c7ccccc7ccc65)cc4)c4ccc5oc6ccccc6c5c4)c32)cc1 ; c1ccc(C2(c3ccccc3)c3ccccc3-c3cccc(N(c4ccccc4-c4cccc5c4oc4c6ccccc6ccc54)c4cccc5sc6ccccc6c45)c32)cc1 ; c1ccc(-n2c3ccccc3c3cc(N(c4cccc(-c5cccc6c5oc5c7ccccc7ccc65)c4)c4ccc5c(c4)-c4ccccc4C5(c4ccccc4)c4ccccc4)ccc32)cc1 ; c1ccc(C2(c3ccccc3)c3ccccc3-c3ccc(N(c4cccc(-c5cccc6c5oc5c7ccccc7ccc65)c4)c4cccc5c4oc4c6ccccc6ccc54)cc32)cc1]

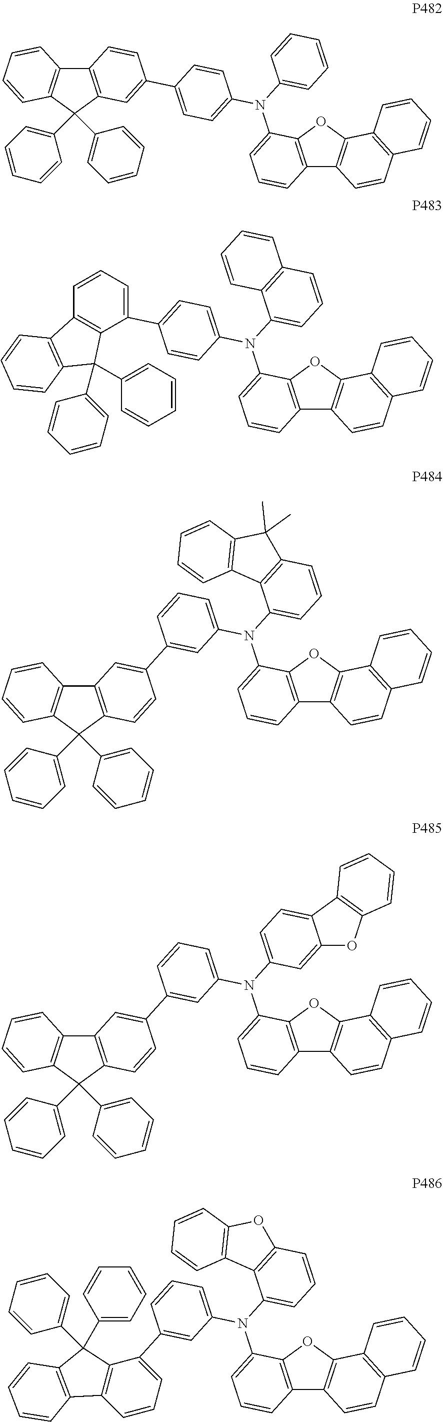

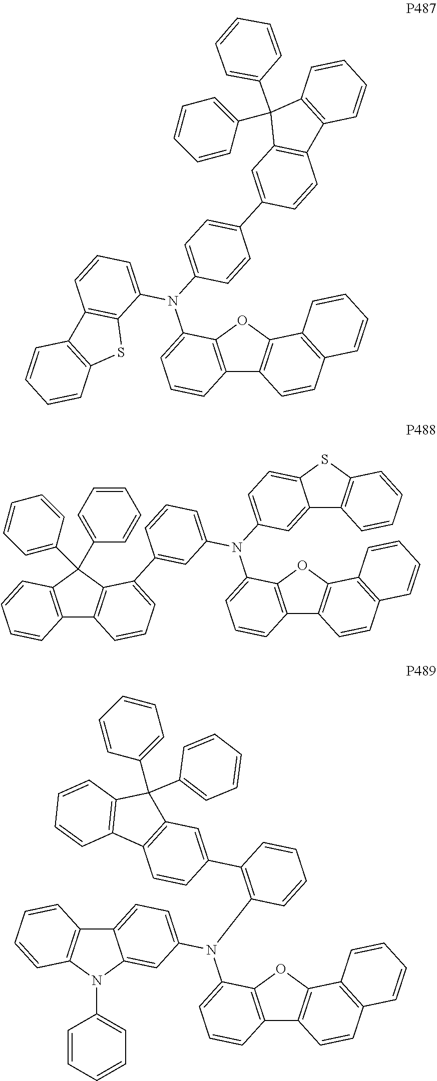 [SMILES: c1ccc(C2(c3ccccc3)c3ccccc3-c3ccc(-c4ccc(N(c5cccc6c5oc5c7ccccc7ccc65)c5cccc6c5sc5ccccc56)cc4)cc32)cc1 ; c1ccc(C2(c3ccccc3)c3ccccc3-c3cccc(-c4cccc(N(c5ccc6sc7ccccc7c6c5)c5cccc6c5oc5c7ccccc7ccc65)c4)c32)cc1 ; c1ccc(-n2c3ccccc3c3ccc(N(c4ccccc4-c4ccc5c(c4)C(c4ccccc4)(c4ccccc4)c4ccccc4-5)c4cccc5c4oc4c6ccccc6ccc54)cc32)cc1]

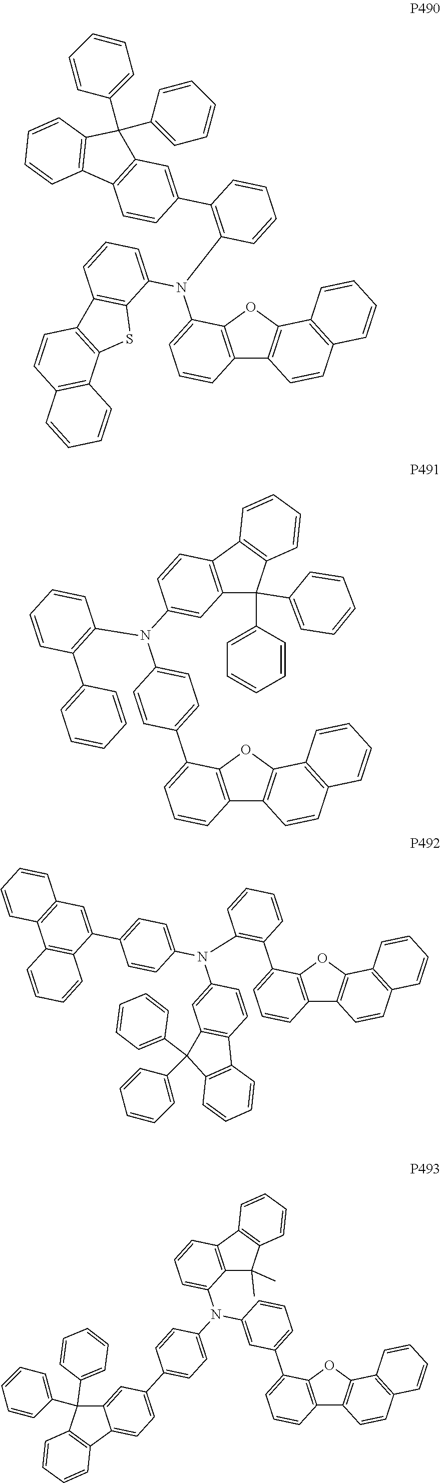 [SMILES: c1ccc(C2(c3ccccc3)c3ccccc3-c3ccc(-c4ccccc4N(c4cccc5c4oc4c6ccccc6ccc54)c4cccc5c4sc4c6ccccc6ccc54)cc32)cc1 ; c1ccc(-c2ccccc2N(c2ccc(-c3cccc4c3oc3c5ccccc5ccc43)cc2)c2ccc3c(c2)C(c2ccccc2)(c2ccccc2)c2ccccc2-3)cc1 ; c1ccc(C2(c3ccccc3)c3ccccc3-c3ccc(N(c4ccc(-c5cc6ccccc6c6ccccc56)cc4)c4ccccc4-c4cccc5c4oc4c6ccccc6ccc54)cc32)cc1 ; CC1(C)c2ccccc2-c2cccc(N(c3ccc(-c4ccc5c(c4)C(c4ccccc4)(c4ccccc4)c4ccccc4-5)cc3)c3cccc(-c4cccc5c4oc4c6ccccc6ccc54)c3)c21]

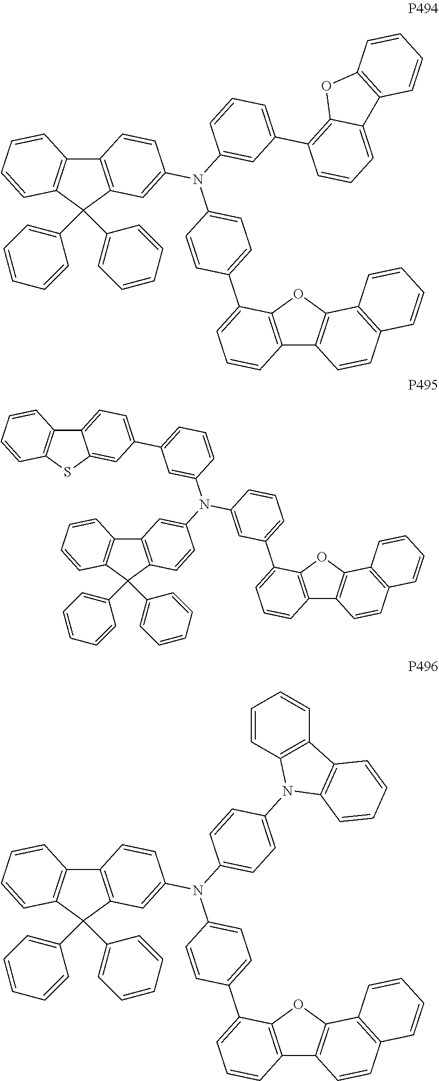 [SMILES: c1ccc(C2(c3ccccc3)c3ccccc3-c3ccc(N(c4ccc(-c5cccc6c5oc5c7ccccc7ccc65)cc4)c4cccc(-c5cccc6c5oc5ccccc56)c4)cc32)cc1 ; c1ccc(C2(c3ccccc3)c3ccccc3-c3cc(N(c4cccc(-c5ccc6c(c5)sc5ccccc56)c4)c4cccc(-c5cccc6c5oc5c7ccccc7ccc65)c4)ccc32)cc1 ; c1ccc(C2(c3ccccc3)c3ccccc3-c3ccc(N(c4ccc(-c5cccc6c5oc5c7ccccc7ccc65)cc4)c4ccc(-n5c6ccccc6c6ccccc65)cc4)cc32)cc1]

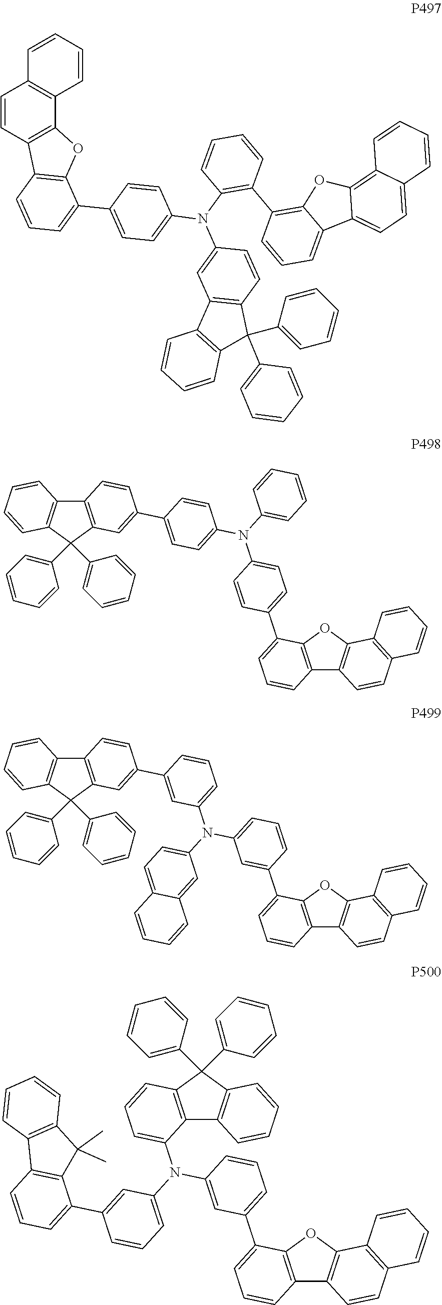 [SMILES: c1ccc(C2(c3ccccc3)c3ccccc3-c3cc(N(c4ccc(-c5cccc6c5oc5c7ccccc7ccc65)cc4)c4ccccc4-c4cccc5c4oc4c6ccccc6ccc54)ccc32)cc1 ; c1ccc(N(c2ccc(-c3ccc4c(c3)C(c3ccccc3)(c3ccccc3)c3ccccc3-4)cc2)c2ccc(-c3cccc4c3oc3c5ccccc5ccc43)cc2)cc1 ; c1ccc(C2(c3ccccc3)c3ccccc3-c3ccc(-c4cccc(N(c5cccc(-c6cccc7c6oc6c8ccccc8ccc76)c5)c5ccc6ccccc6c5)c4)cc32)cc1 ; CC1(C)c2ccccc2-c2cccc(-c3cccc(N(c4cccc(-c5cccc6c5oc5c7ccccc7ccc65)c4)c4cccc5c4-c4ccccc4C5(c4ccccc4)c4ccccc4)c3)c21]

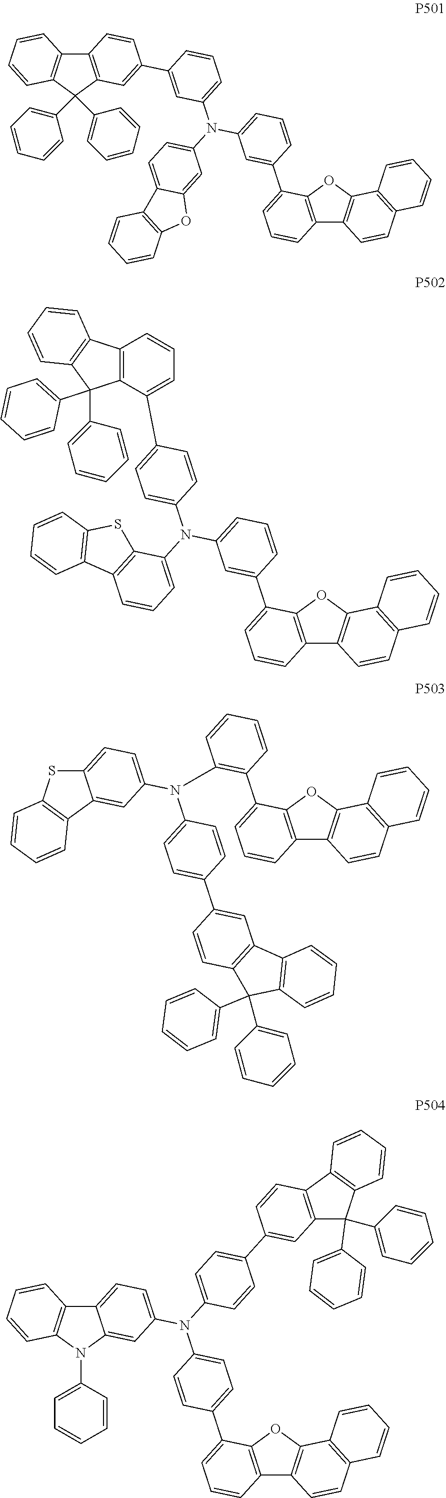 [SMILES: c1ccc(C2(c3ccccc3)c3ccccc3-c3ccc(-c4cccc(N(c5cccc(-c6cccc7c6oc6c8ccccc8ccc76)c5)c5ccc6c(c5)oc5ccccc56)c4)cc32)cc1 ; c1ccc(C2(c3ccccc3)c3ccccc3-c3cccc(-c4ccc(N(c5cccc(-c6cccc7c6oc6c8ccccc8ccc76)c5)c5cccc6c5sc5ccccc56)cc4)c32)cc1 ; c1ccc(C2(c3ccccc3)c3ccccc3-c3cc(-c4ccc(N(c5ccc6sc7ccccc7c6c5)c5ccccc5-c5cccc6c5oc5c7ccccc7ccc65)cc4)ccc32)cc1 ; c1ccc(-n2c3ccccc3c3ccc(N(c4ccc(-c5ccc6c(c5)C(c5ccccc5)(c5ccccc5)c5ccccc5-6)cc4)c4ccc(-c5cccc6c5oc5c7ccccc7ccc65)cc4)cc32)cc1]

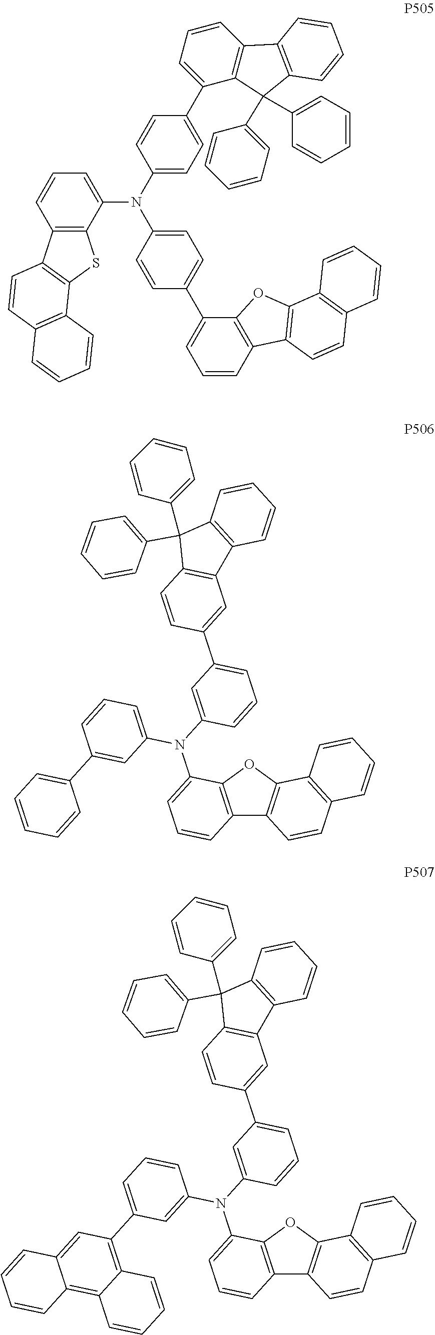 [SMILES: c1ccc(C2(c3ccccc3)c3ccccc3-c3cccc(-c4ccc(N(c5ccc(-c6cccc7c6oc6c8ccccc8ccc76)cc5)c5cccc6c5sc5c7ccccc7ccc65)cc4)c32)cc1 ; c1ccc(-c2cccc(N(c3cccc(-c4ccc5c(c4)-c4ccccc4C5(c4ccccc4)c4ccccc4)c3)c3cccc4c3oc3c5ccccc5ccc43)c2)cc1 ; c1ccc(C2(c3ccccc3)c3ccccc3-c3cc(-c4cccc(N(c5cccc(-c6cc7ccccc7c7ccccc67)c5)c5cccc6c5oc5c7ccccc7ccc65)c4)ccc32)cc1]

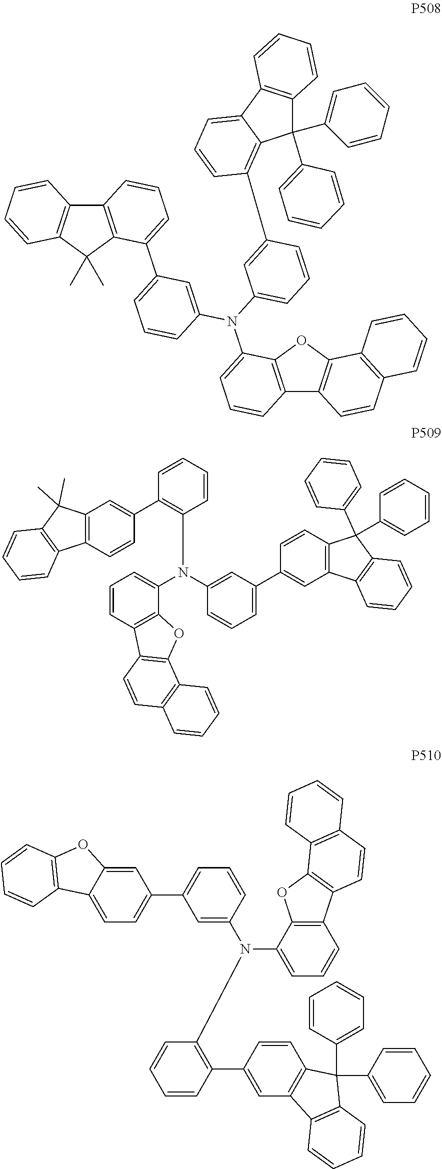 [SMILES: CC1(C)c2ccccc2-c2cccc(-c3cccc(N(c4cccc(-c5cccc6c5C(c5ccccc5)(c5ccccc5)c5ccccc5-6)c4)c4cccc5c4oc4c6ccccc6ccc54)c3)c21 ; CC1(C)c2ccccc2-c2ccc(-c3ccccc3N(c3cccc(-c4ccc5c(c4)-c4ccccc4C5(c4ccccc4)c4ccccc4)c3)c3cccc4c3oc3c5ccccc5ccc43)cc21 ; c1ccc(C2(c3ccccc3)c3ccccc3-c3cc(-c4ccccc4N(c4cccc(-c5ccc6c(c5)oc5ccccc56)c4)c4cccc5c4oc4c6ccccc6ccc54)ccc32)cc1]

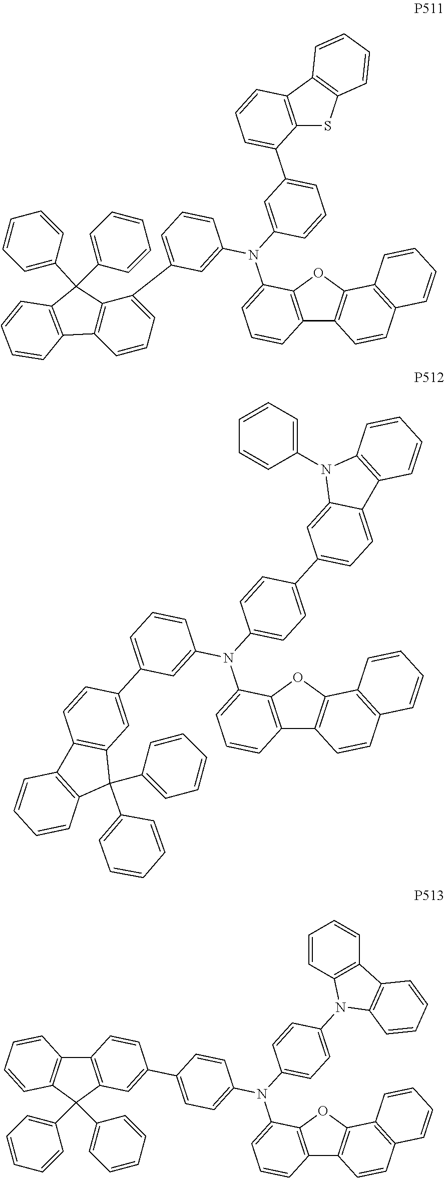 [SMILES: c1ccc(C2(c3ccccc3)c3ccccc3-c3cccc(-c4cccc(N(c5cccc(-c6cccc7c6sc6ccccc67)c5)c5cccc6c5oc5c7ccccc7ccc65)c4)c32)cc1 ; c1ccc(-n2c3ccccc3c3ccc(-c4ccc(N(c5cccc(-c6ccc7c(c6)C(c6ccccc6)(c6ccccc6)c6ccccc6-7)c5)c5cccc6c5oc5c7ccccc7ccc65)cc4)cc32)cc1 ; c1ccc(C2(c3ccccc3)c3ccccc3-c3ccc(-c4ccc(N(c5ccc(-n6c7ccccc7c7ccccc76)cc5)c5cccc6c5oc5c7ccccc7ccc65)cc4)cc32)cc1]

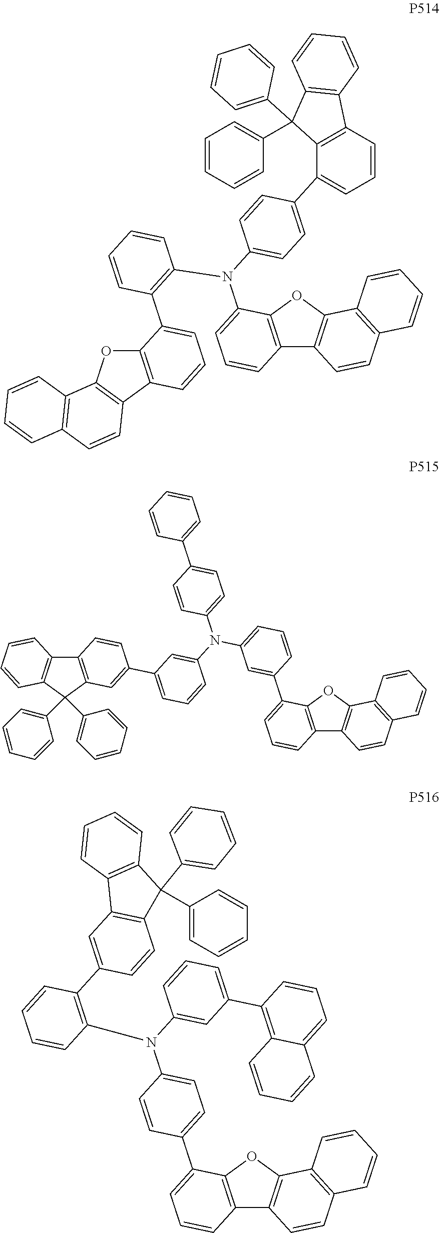 [SMILES: c1ccc(C2(c3ccccc3)c3ccccc3-c3cccc(-c4ccc(N(c5ccccc5-c5cccc6c5oc5c7ccccc7ccc65)c5cccc6c5oc5c7ccccc7ccc65)cc4)c32)cc1 ; c1ccc(-c2ccc(N(c3cccc(-c4ccc5c(c4)C(c4ccccc4)(c4ccccc4)c4ccccc4-5)c3)c3cccc(-c4cccc5c4oc4c6ccccc6ccc54)c3)cc2)cc1 ; c1ccc(C2(c3ccccc3)c3ccccc3-c3cc(-c4ccccc4N(c4ccc(-c5cccc6c5oc5c7ccccc7ccc65)cc4)c4cccc(-c5cccc6ccccc56)c4)ccc32)cc1]

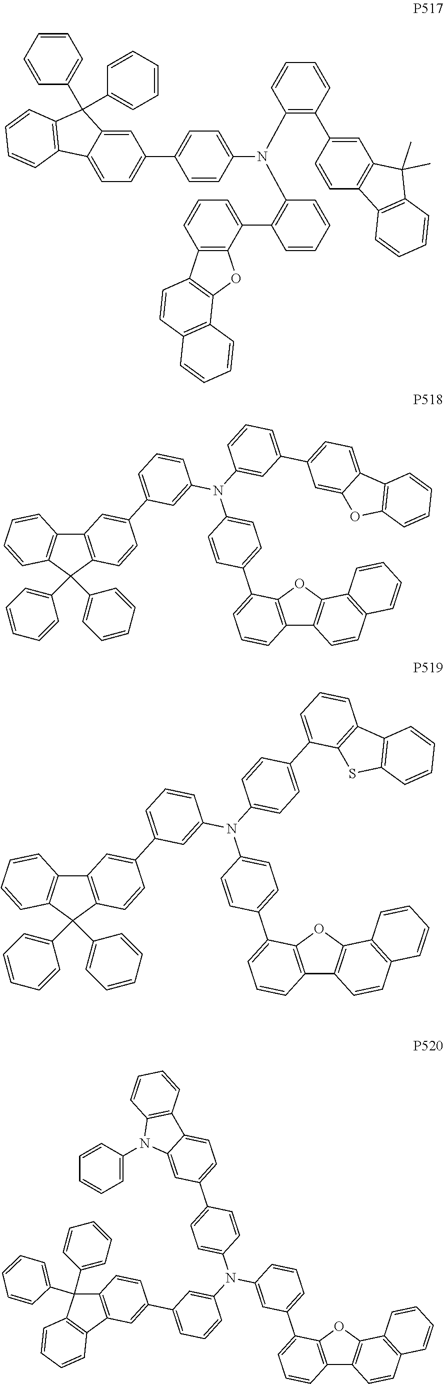

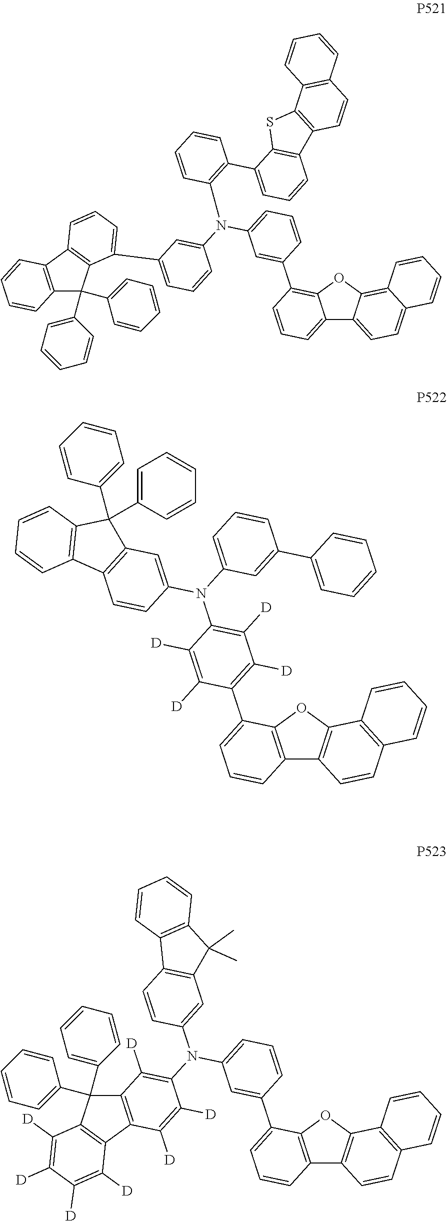 [SMILES: c1ccc(C2(c3ccccc3)c3ccccc3-c3cccc(-c4cccc(N(c5cccc(-c6cccc7c6oc6c8ccccc8ccc76)c5)c5ccccc5-c5cccc6c5sc5c7ccccc7ccc65)c4)c32)cc1 ; [2H]c1c([2H])c(N(c2cccc(-c3ccccc3)c2)c2ccc3c(c2)C(c2ccccc2)(c2ccccc2)c2ccccc2-3)c([2H])c([2H])c1-c1cccc2c1oc1c3ccccc3ccc21 ; [2H]c1c([2H])c([2H])c2c(c1[2H])-c1c([2H])c([2H])c(N(c3cccc(-c4cccc5c4oc4c6ccccc6ccc54)c3)c3ccc4c(c3)C(C)(C)c3ccccc3-4)c([2H])c1C2(c1ccccc1)c1ccccc1]

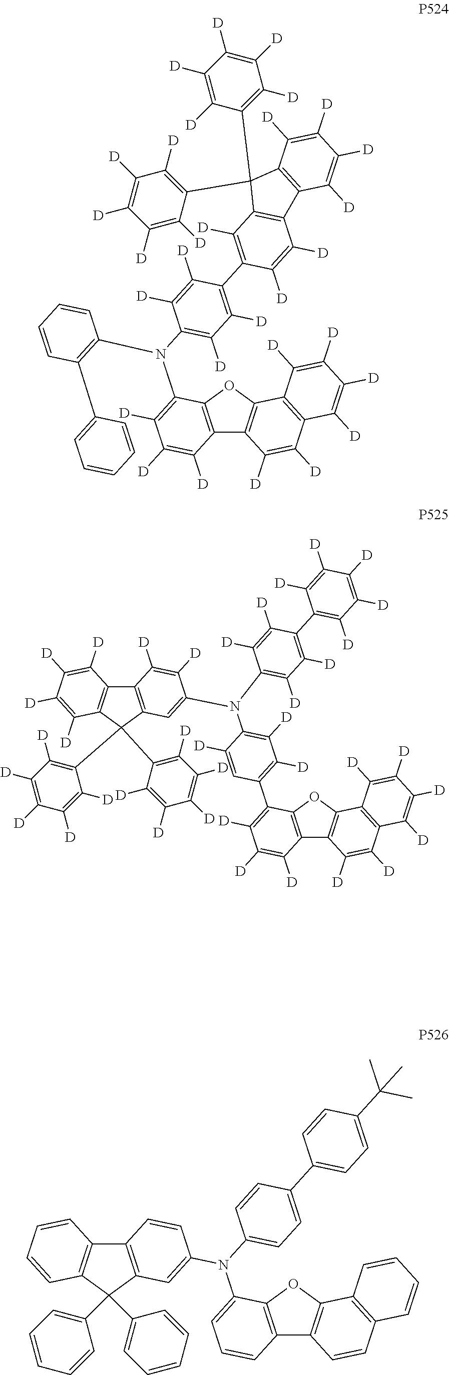

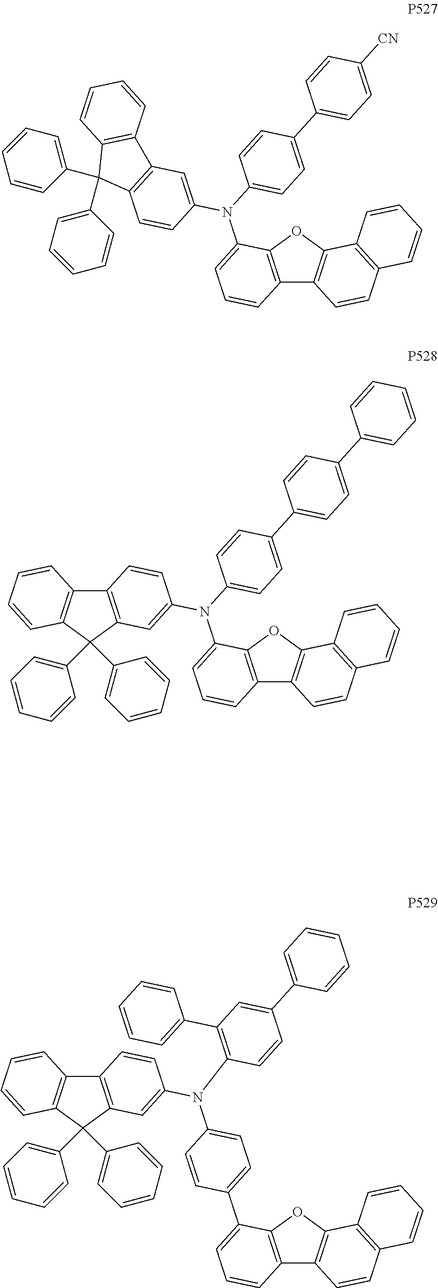 [SMILES: N#Cc1ccc(-c2ccc(N(c3ccc4c(c3)-c3ccccc3C4(c3ccccc3)c3ccccc3)c3cccc4c3oc3c5ccccc5ccc43)cc2)cc1 ; c1ccc(-c2ccc(-c3ccc(N(c4ccc5c(c4)C(c4ccccc4)(c4ccccc4)c4ccccc4-5)c4cccc5c4oc4c6ccccc6ccc54)cc3)cc2)cc1 ; c1ccc(-c2ccc(N(c3ccc(-c4cccc5c4oc4c6ccccc6ccc54)cc3)c3ccc4c(c3)C(c3ccccc3)(c3ccccc3)c3ccccc3-4)c(-c3ccccc3)c2)cc1]

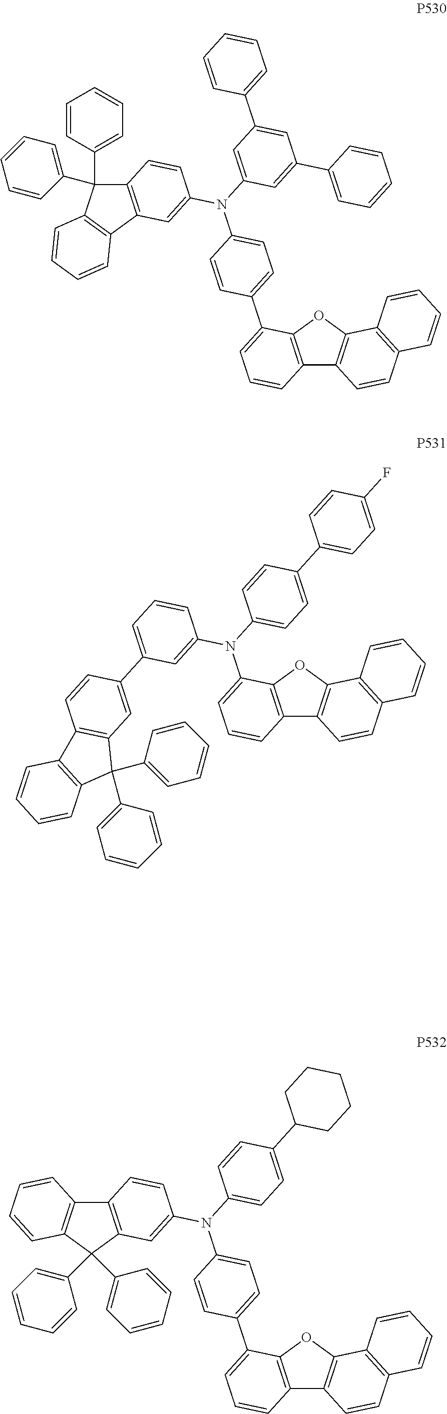 [SMILES: c1ccc(-c2cc(-c3ccccc3)cc(N(c3ccc(-c4cccc5c4oc4c6ccccc6ccc54)cc3)c3ccc4c(c3)-c3ccccc3C4(c3ccccc3)c3ccccc3)c2)cc1 ; Fc1ccc(-c2ccc(N(c3cccc(-c4ccc5c(c4)C(c4ccccc4)(c4ccccc4)c4ccccc4-5)c3)c3cccc4c3oc3c5ccccc5ccc43)cc2)cc1 ; c1ccc(C2(c3ccccc3)c3ccccc3-c3ccc(N(c4ccc(-c5cccc6c5oc5c7ccccc7ccc65)cc4)c4ccc(C5CCCCC5)cc4)cc32)cc1]

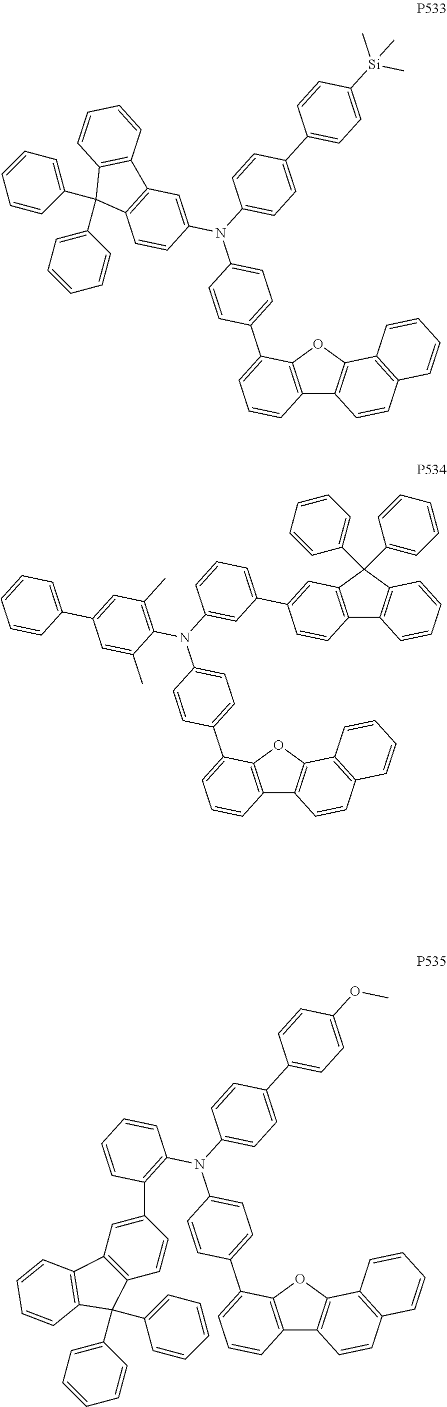 [SMILES: C[Si](C)(C)c1ccc(-c2ccc(N(c3ccc(-c4cccc5c4oc4c6ccccc6ccc54)cc3)c3ccc4c(c3)-c3ccccc3C4(c3ccccc3)c3ccccc3)cc2)cc1 ; Cc1cc(-c2ccccc2)cc(C)c1N(c1ccc(-c2cccc3c2oc2c4ccccc4ccc32)cc1)c1cccc(-c2ccc3c(c2)C(c2ccccc2)(c2ccccc2)c2ccccc2-3)c1 ; COc1ccc(-c2ccc(N(c3ccc(-c4cccc5c4oc4c6ccccc6ccc54)cc3)c3ccccc3-c3ccc4c(c3)-c3ccccc3C4(c3ccccc3)c3ccccc3)cc2)cc1]

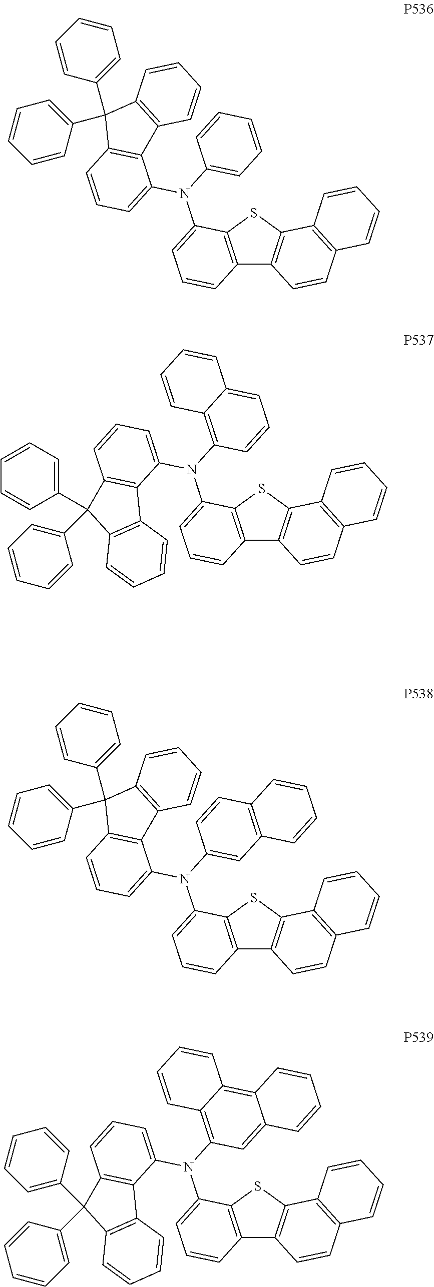 [SMILES: c1ccc(N(c2cccc3c2-c2ccccc2C3(c2ccccc2)c2ccccc2)c2cccc3c2sc2c4ccccc4ccc32)cc1 ; c1ccc(C2(c3ccccc3)c3ccccc3-c3c(N(c4cccc5ccccc45)c4cccc5c4sc4c6ccccc6ccc54)cccc32)cc1 ; c1ccc(C2(c3ccccc3)c3ccccc3-c3c(N(c4ccc5ccccc5c4)c4cccc5c4sc4c6ccccc6ccc54)cccc32)cc1 ; c1ccc(C2(c3ccccc3)c3ccccc3-c3c(N(c4cc5ccccc5c5ccccc45)c4cccc5c4sc4c6ccccc6ccc54)cccc32)cc1]

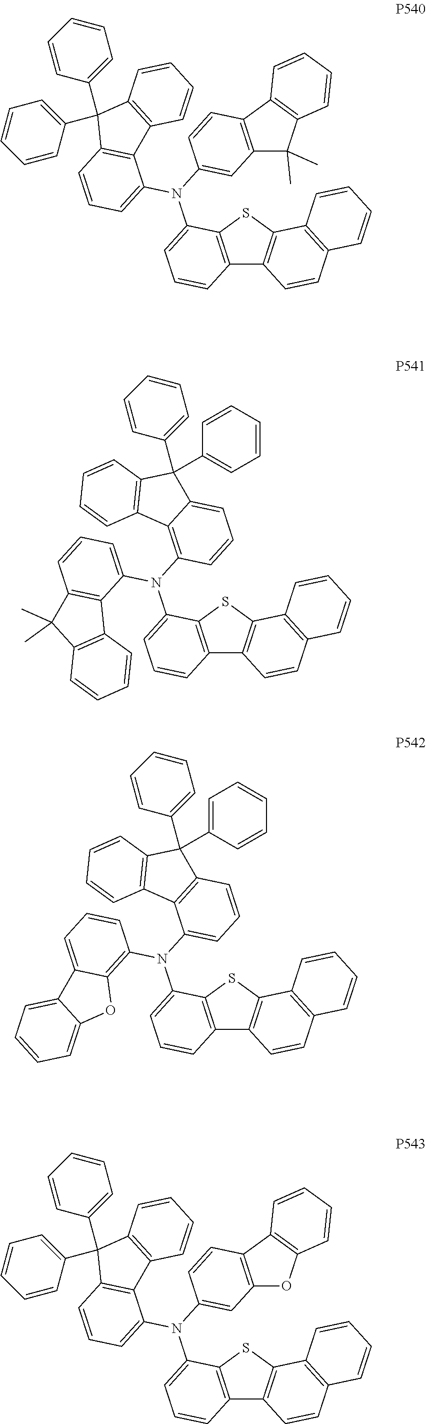 [SMILES: CC1(C)c2ccccc2-c2ccc(N(c3cccc4c3-c3ccccc3C4(c3ccccc3)c3ccccc3)c3cccc4c3sc3c5ccccc5ccc43)cc21 ; CC1(C)c2ccccc2-c2c(N(c3cccc4c3-c3ccccc3C4(c3ccccc3)c3ccccc3)c3cccc4c3sc3c5ccccc5ccc43)cccc21 ; c1ccc(C2(c3ccccc3)c3ccccc3-c3c(N(c4cccc5c4oc4ccccc45)c4cccc5c4sc4c6ccccc6ccc54)cccc32)cc1 ; c1ccc(C2(c3ccccc3)c3ccccc3-c3c(N(c4ccc5c(c4)oc4ccccc45)c4cccc5c4sc4c6ccccc6ccc54)cccc32)cc1]

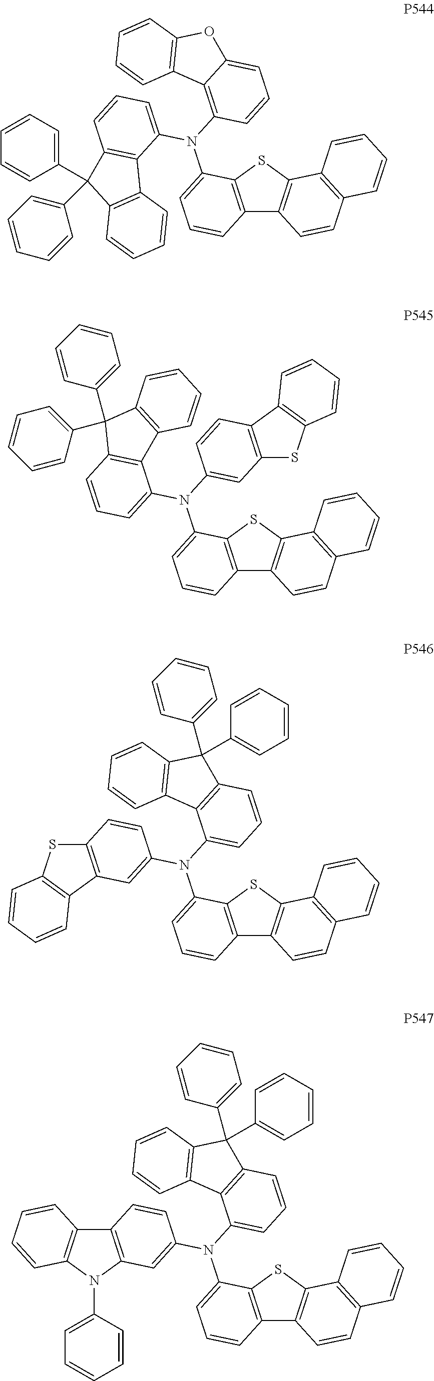 [SMILES: c1ccc(C2(c3ccccc3)c3ccccc3-c3c(N(c4cccc5c4sc4c6ccccc6ccc54)c4cccc5oc6ccccc6c45)cccc32)cc1 ; c1ccc(C2(c3ccccc3)c3ccccc3-c3c(N(c4ccc5c(c4)sc4ccccc45)c4cccc5c4sc4c6ccccc6ccc54)cccc32)cc1 ; c1ccc(C2(c3ccccc3)c3ccccc3-c3c(N(c4ccc5sc6ccccc6c5c4)c4cccc5c4sc4c6ccccc6ccc54)cccc32)cc1 ; c1ccc(-n2c3ccccc3c3ccc(N(c4cccc5c4-c4ccccc4C5(c4ccccc4)c4ccccc4)c4cccc5c4sc4c6ccccc6ccc54)cc32)cc1]

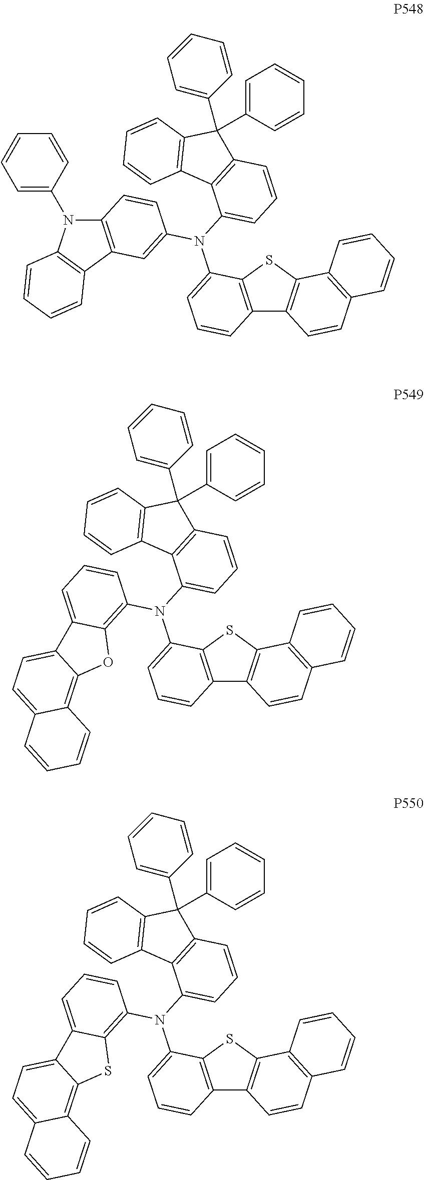 [SMILES: c1ccc(-n2c3ccccc3c3cc(N(c4cccc5c4-c4ccccc4C5(c4ccccc4)c4ccccc4)c4cccc5c4sc4c6ccccc6ccc54)ccc32)cc1 ; c1ccc(C2(c3ccccc3)c3ccccc3-c3c(N(c4cccc5c4oc4c6ccccc6ccc54)c4cccc5c4sc4c6ccccc6ccc54)cccc32)cc1 ; c1ccc(C2(c3ccccc3)c3ccccc3-c3c(N(c4cccc5c4sc4c6ccccc6ccc54)c4cccc5c4sc4c6ccccc6ccc54)cccc32)cc1]

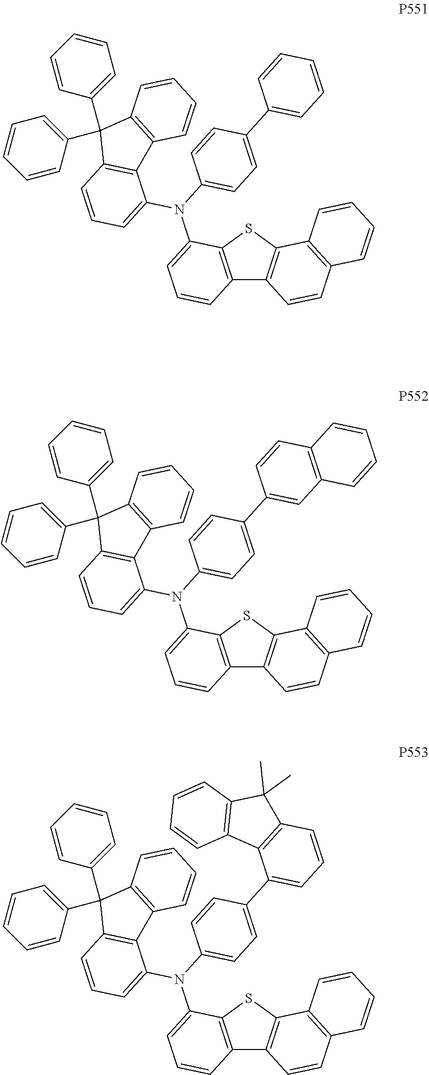 [SMILES: c1ccc(-c2ccc(N(c3cccc4c3-c3ccccc3C4(c3ccccc3)c3ccccc3)c3cccc4c3sc3c5ccccc5ccc43)cc2)cc1 ; c1ccc(C2(c3ccccc3)c3ccccc3-c3c(N(c4ccc(-c5ccc6ccccc6c5)cc4)c4cccc5c4sc4c6ccccc6ccc54)cccc32)cc1 ; CC1(C)c2ccccc2-c2c(-c3ccc(N(c4cccc5c4-c4ccccc4C5(c4ccccc4)c4ccccc4)c4cccc5c4sc4c6ccccc6ccc54)cc3)cccc21]

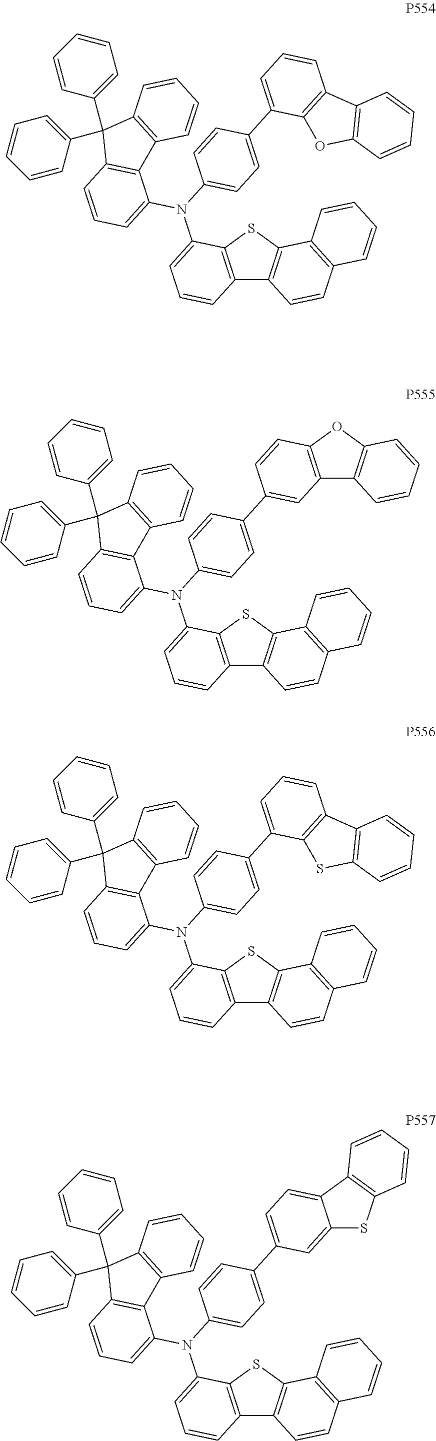 [SMILES: c1ccc(C2(c3ccccc3)c3ccccc3-c3c(N(c4ccc(-c5cccc6c5oc5ccccc56)cc4)c4cccc5c4sc4c6ccccc6ccc54)cccc32)cc1 ; c1ccc(C2(c3ccccc3)c3ccccc3-c3c(N(c4ccc(-c5ccc6oc7ccccc7c6c5)cc4)c4cccc5c4sc4c6ccccc6ccc54)cccc32)cc1 ; c1ccc(C2(c3ccccc3)c3ccccc3-c3c(N(c4ccc(-c5cccc6c5sc5ccccc56)cc4)c4cccc5c4sc4c6ccccc6ccc54)cccc32)cc1 ; c1ccc(C2(c3ccccc3)c3ccccc3-c3c(N(c4ccc(-c5ccc6c(c5)sc5ccccc56)cc4)c4cccc5c4sc4c6ccccc6ccc54)cccc32)cc1]

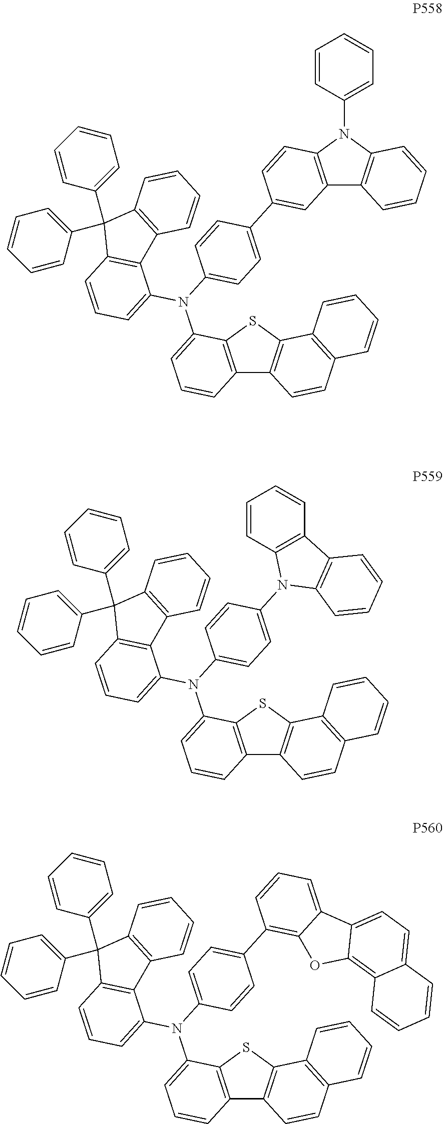 [SMILES: c1ccc(-n2c3ccccc3c3cc(-c4ccc(N(c5cccc6c5-c5ccccc5C6(c5ccccc5)c5ccccc5)c5cccc6c5sc5c7ccccc7ccc65)cc4)ccc32)cc1 ; c1ccc(C2(c3ccccc3)c3ccccc3-c3c(N(c4ccc(-n5c6ccccc6c6ccccc65)cc4)c4cccc5c4sc4c6ccccc6ccc54)cccc32)cc1 ; c1ccc(C2(c3ccccc3)c3ccccc3-c3c(N(c4ccc(-c5cccc6c5oc5c7ccccc7ccc65)cc4)c4cccc5c4sc4c6ccccc6ccc54)cccc32)cc1]

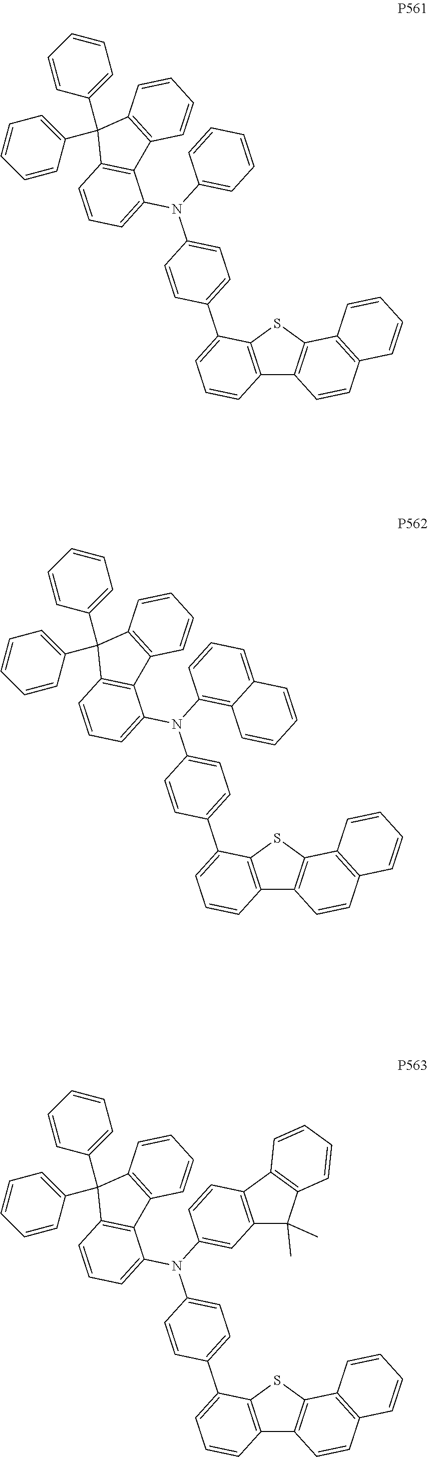 [SMILES: c1ccc(N(c2ccc(-c3cccc4c3sc3c5ccccc5ccc43)cc2)c2cccc3c2-c2ccccc2C3(c2ccccc2)c2ccccc2)cc1 ; c1ccc(C2(c3ccccc3)c3ccccc3-c3c(N(c4ccc(-c5cccc6c5sc5c7ccccc7ccc65)cc4)c4cccc5ccccc45)cccc32)cc1 ; CC1(C)c2ccccc2-c2ccc(N(c3ccc(-c4cccc5c4sc4c6ccccc6ccc54)cc3)c3cccc4c3-c3ccccc3C4(c3ccccc3)c3ccccc3)cc21]

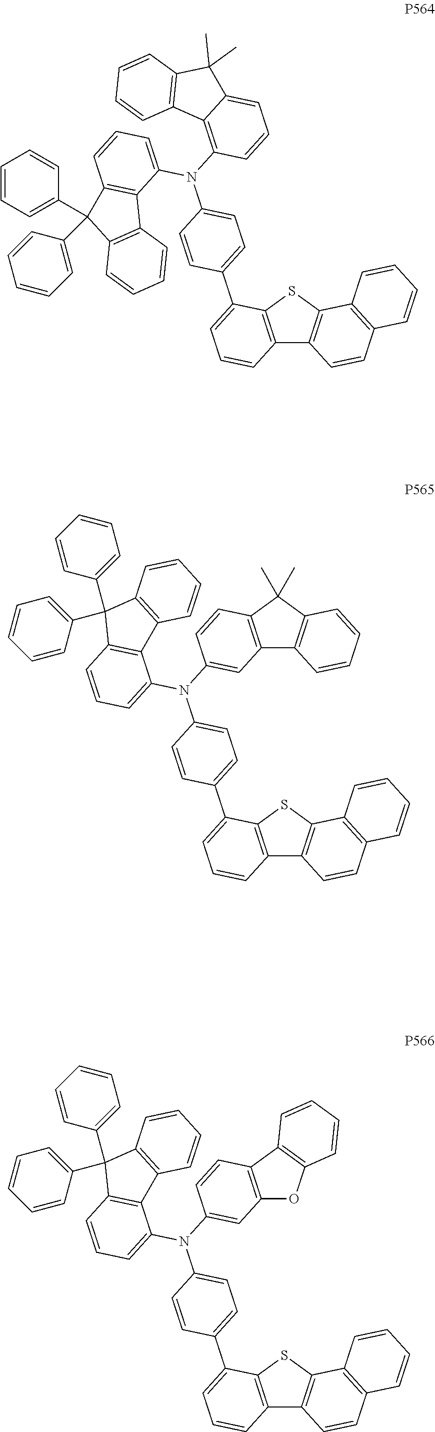 [SMILES: CC1(C)c2ccccc2-c2c(N(c3ccc(-c4cccc5c4sc4c6ccccc6ccc54)cc3)c3cccc4c3-c3ccccc3C4(c3ccccc3)c3ccccc3)cccc21 ; CC1(C)c2ccccc2-c2cc(N(c3ccc(-c4cccc5c4sc4c6ccccc6ccc54)cc3)c3cccc4c3-c3ccccc3C4(c3ccccc3)c3ccccc3)ccc21 ; c1ccc(C2(c3ccccc3)c3ccccc3-c3c(N(c4ccc(-c5cccc6c5sc5c7ccccc7ccc65)cc4)c4ccc5c(c4)oc4ccccc45)cccc32)cc1]

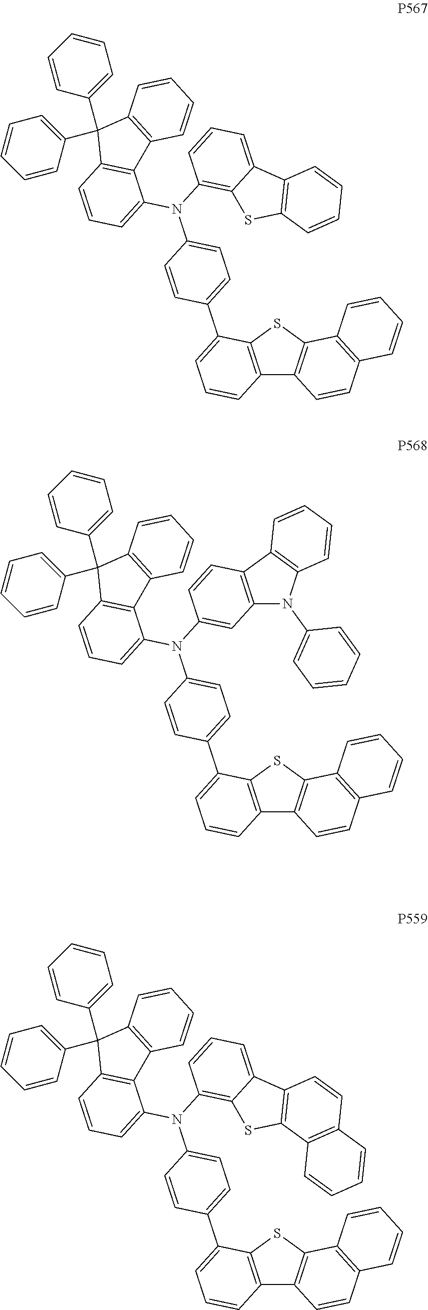 [SMILES: c1ccc(C2(c3ccccc3)c3ccccc3-c3c(N(c4ccc(-c5cccc6c5sc5c7ccccc7ccc65)cc4)c4cccc5c4sc4ccccc45)cccc32)cc1 ; c1ccc(-n2c3ccccc3c3ccc(N(c4ccc(-c5cccc6c5sc5c7ccccc7ccc65)cc4)c4cccc5c4-c4ccccc4C5(c4ccccc4)c4ccccc4)cc32)cc1 ; c1ccc(C2(c3ccccc3)c3ccccc3-c3c(N(c4ccc(-c5cccc6c5sc5c7ccccc7ccc65)cc4)c4cccc5c4sc4c6ccccc6ccc54)cccc32)cc1]

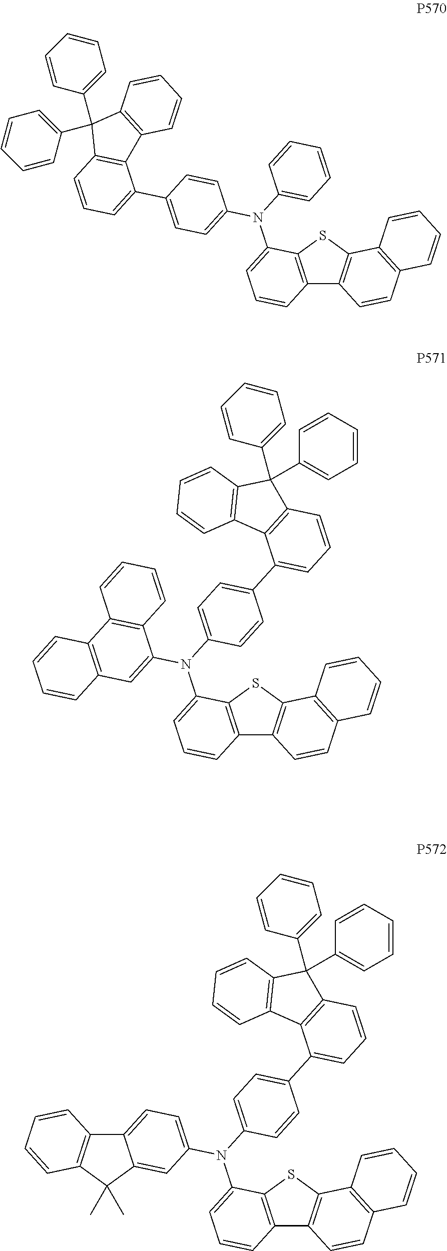 [SMILES: c1ccc(N(c2ccc(-c3cccc4c3-c3ccccc3C4(c3ccccc3)c3ccccc3)cc2)c2cccc3c2sc2c4ccccc4ccc32)cc1 ; c1ccc(C2(c3ccccc3)c3ccccc3-c3c(-c4ccc(N(c5cc6ccccc6c6ccccc56)c5cccc6c5sc5c7ccccc7ccc65)cc4)cccc32)cc1 ; CC1(C)c2ccccc2-c2ccc(N(c3ccc(-c4cccc5c4-c4ccccc4C5(c4ccccc4)c4ccccc4)cc3)c3cccc4c3sc3c5ccccc5ccc43)cc21]

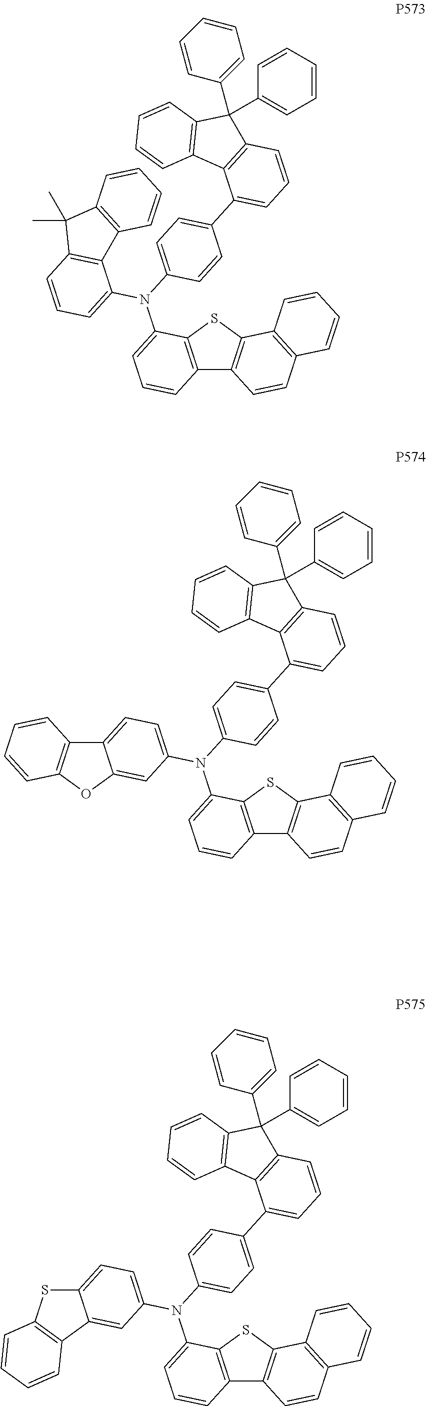 [SMILES: CC1(C)c2ccccc2-c2c(N(c3ccc(-c4cccc5c4-c4ccccc4C5(c4ccccc4)c4ccccc4)cc3)c3cccc4c3sc3c5ccccc5ccc43)cccc21 ; c1ccc(C2(c3ccccc3)c3ccccc3-c3c(-c4ccc(N(c5ccc6c(c5)oc5ccccc56)c5cccc6c5sc5c7ccccc7ccc65)cc4)cccc32)cc1 ; c1ccc(C2(c3ccccc3)c3ccccc3-c3c(-c4ccc(N(c5ccc6sc7ccccc7c6c5)c5cccc6c5sc5c7ccccc7ccc65)cc4)cccc32)cc1]

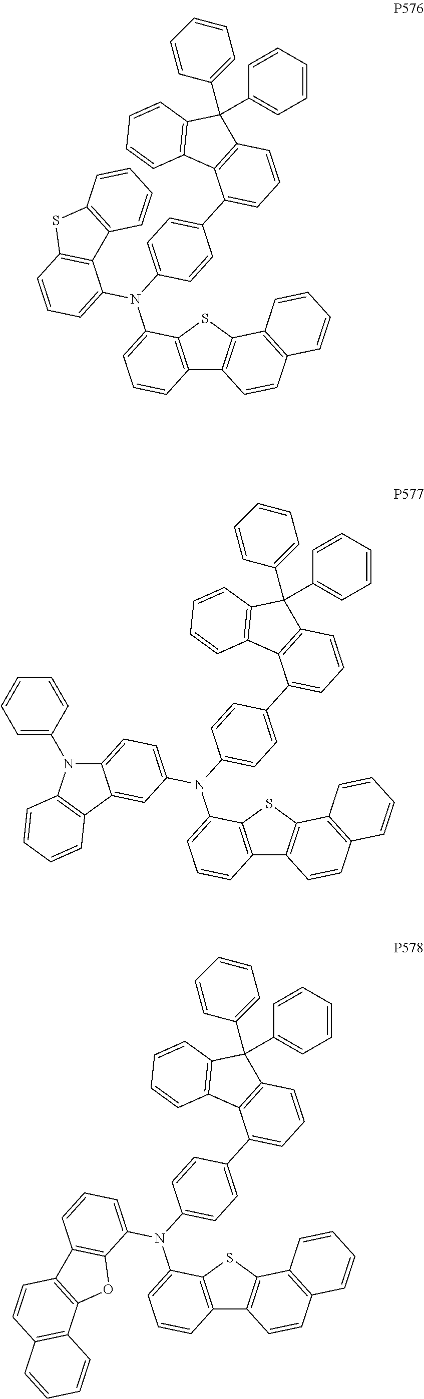 [SMILES: c1ccc(C2(c3ccccc3)c3ccccc3-c3c(-c4ccc(N(c5cccc6c5sc5c7ccccc7ccc65)c5cccc6sc7ccccc7c56)cc4)cccc32)cc1 ; c1ccc(-n2c3ccccc3c3cc(N(c4ccc(-c5cccc6c5-c5ccccc5C6(c5ccccc5)c5ccccc5)cc4)c4cccc5c4sc4c6ccccc6ccc54)ccc32)cc1 ; c1ccc(C2(c3ccccc3)c3ccccc3-c3c(-c4ccc(N(c5cccc6c5oc5c7ccccc7ccc65)c5cccc6c5sc5c7ccccc7ccc65)cc4)cccc32)cc1]

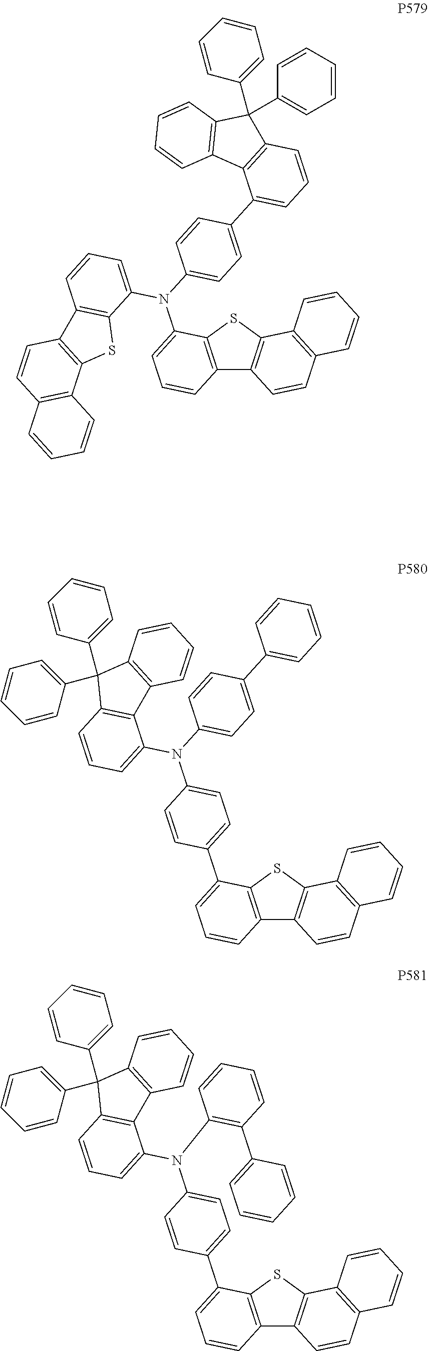 [SMILES: c1ccc(C2(c3ccccc3)c3ccccc3-c3c(-c4ccc(N(c5cccc6c5sc5c7ccccc7ccc65)c5cccc6c5sc5c7ccccc7ccc65)cc4)cccc32)cc1 ; c1ccc(-c2ccc(N(c3ccc(-c4cccc5c4sc4c6ccccc6ccc54)cc3)c3cccc4c3-c3ccccc3C4(c3ccccc3)c3ccccc3)cc2)cc1 ; c1ccc(-c2ccccc2N(c2ccc(-c3cccc4c3sc3c5ccccc5ccc43)cc2)c2cccc3c2-c2ccccc2C3(c2ccccc2)c2ccccc2)cc1]

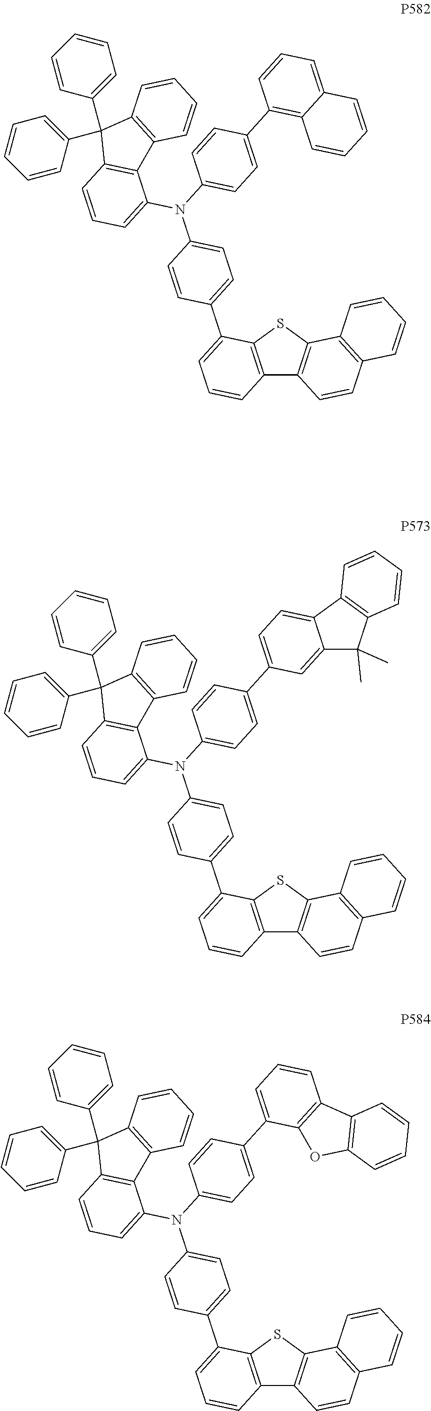 [SMILES: c1ccc(C2(c3ccccc3)c3ccccc3-c3c(N(c4ccc(-c5cccc6ccccc56)cc4)c4ccc(-c5cccc6c5sc5c7ccccc7ccc65)cc4)cccc32)cc1 ; CC1(C)c2ccccc2-c2ccc(-c3ccc(N(c4ccc(-c5cccc6c5sc5c7ccccc7ccc65)cc4)c4cccc5c4-c4ccccc4C5(c4ccccc4)c4ccccc4)cc3)cc21 ; c1ccc(C2(c3ccccc3)c3ccccc3-c3c(N(c4ccc(-c5cccc6c5oc5ccccc56)cc4)c4ccc(-c5cccc6c5sc5c7ccccc7ccc65)cc4)cccc32)cc1]

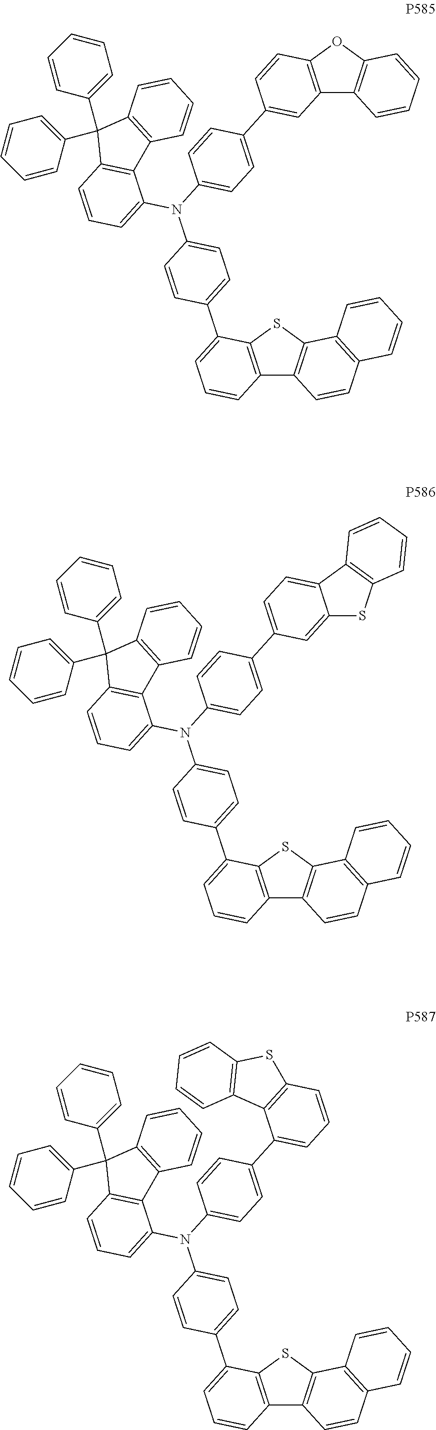 [SMILES: c1ccc(C2(c3ccccc3)c3ccccc3-c3c(N(c4ccc(-c5ccc6oc7ccccc7c6c5)cc4)c4ccc(-c5cccc6c5sc5c7ccccc7ccc65)cc4)cccc32)cc1 ; c1ccc(C2(c3ccccc3)c3ccccc3-c3c(N(c4ccc(-c5ccc6c(c5)sc5ccccc56)cc4)c4ccc(-c5cccc6c5sc5c7ccccc7ccc65)cc4)cccc32)cc1 ; c1ccc(C2(c3ccccc3)c3ccccc3-c3c(N(c4ccc(-c5cccc6c5sc5c7ccccc7ccc65)cc4)c4ccc(-c5cccc6sc7ccccc7c56)cc4)cccc32)cc1]

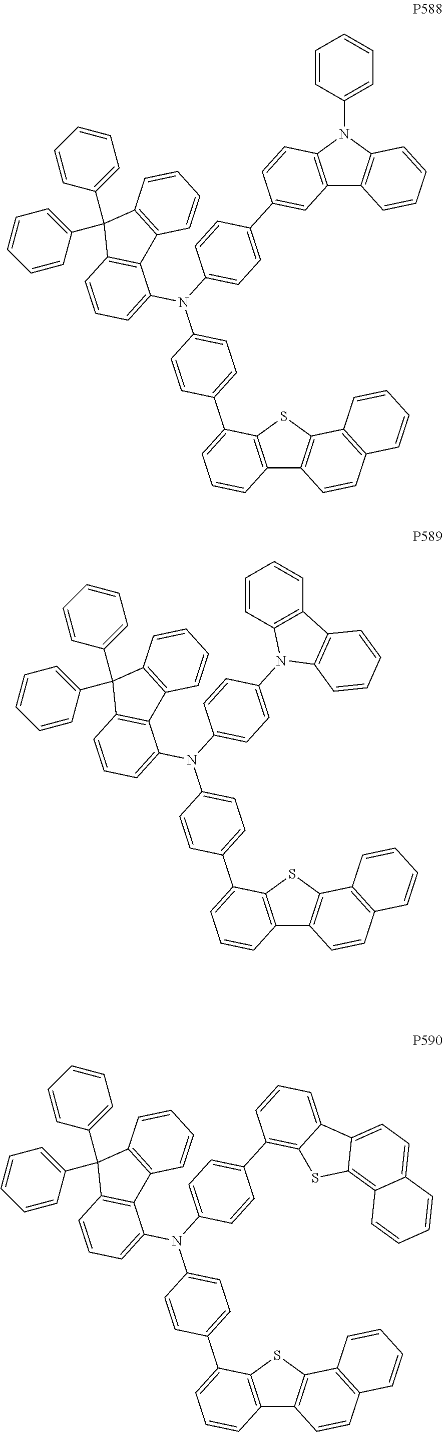 [SMILES: c1ccc(-n2c3ccccc3c3cc(-c4ccc(N(c5ccc(-c6cccc7c6sc6c8ccccc8ccc76)cc5)c5cccc6c5-c5ccccc5C6(c5ccccc5)c5ccccc5)cc4)ccc32)cc1 ; c1ccc(C2(c3ccccc3)c3ccccc3-c3c(N(c4ccc(-c5cccc6c5sc5c7ccccc7ccc65)cc4)c4ccc(-n5c6ccccc6c6ccccc65)cc4)cccc32)cc1 ; c1ccc(C2(c3ccccc3)c3ccccc3-c3c(N(c4ccc(-c5cccc6c5sc5c7ccccc7ccc65)cc4)c4ccc(-c5cccc6c5sc5c7ccccc7ccc65)cc4)cccc32)cc1]

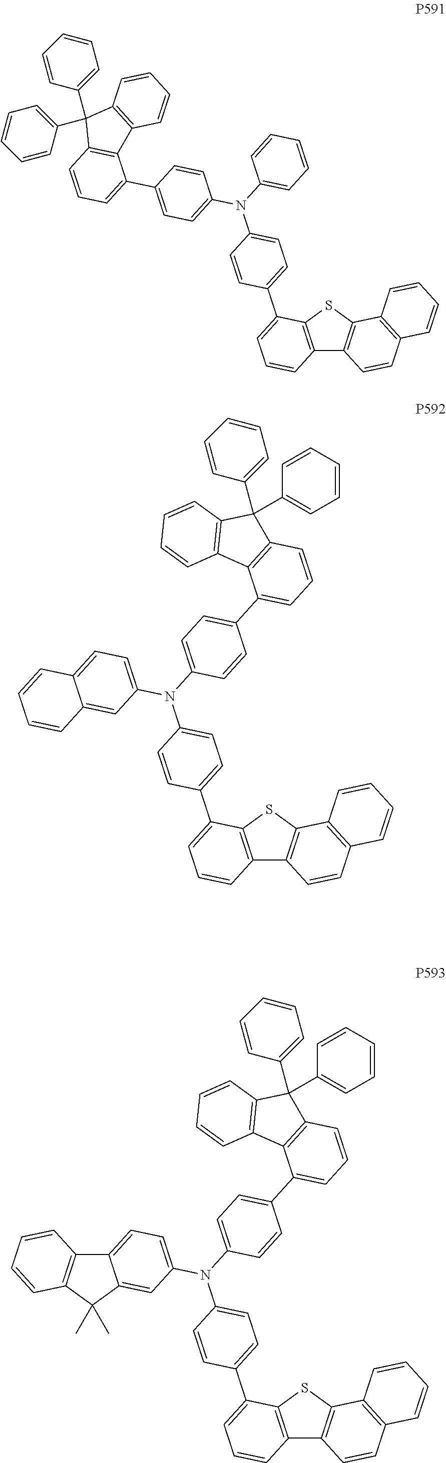 [SMILES: c1ccc(N(c2ccc(-c3cccc4c3-c3ccccc3C4(c3ccccc3)c3ccccc3)cc2)c2ccc(-c3cccc4c3sc3c5ccccc5ccc43)cc2)cc1 ; c1ccc(C2(c3ccccc3)c3ccccc3-c3c(-c4ccc(N(c5ccc(-c6cccc7c6sc6c8ccccc8ccc76)cc5)c5ccc6ccccc6c5)cc4)cccc32)cc1 ; CC1(C)c2ccccc2-c2ccc(N(c3ccc(-c4cccc5c4-c4ccccc4C5(c4ccccc4)c4ccccc4)cc3)c3ccc(-c4cccc5c4sc4c6ccccc6ccc54)cc3)cc21]

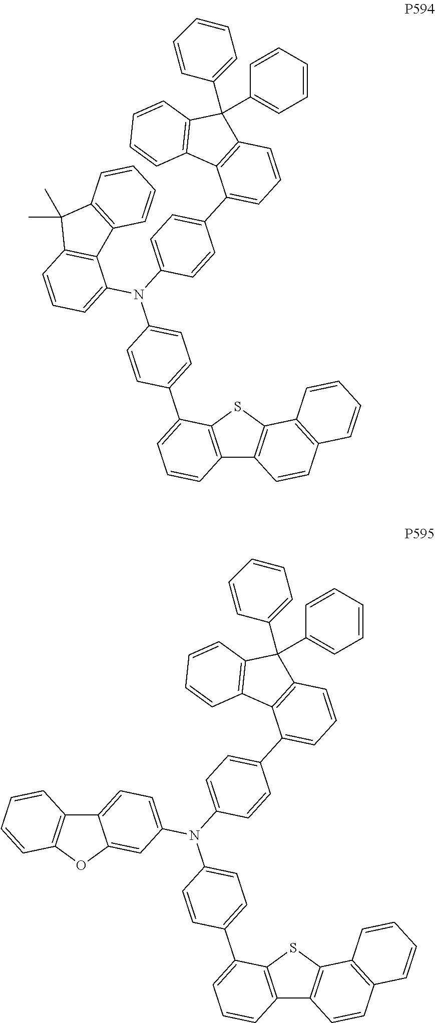 [SMILES: CC1(C)c2ccccc2-c2c(N(c3ccc(-c4cccc5c4-c4ccccc4C5(c4ccccc4)c4ccccc4)cc3)c3ccc(-c4cccc5c4sc4c6ccccc6ccc54)cc3)cccc21 ; c1ccc(C2(c3ccccc3)c3ccccc3-c3c(-c4ccc(N(c5ccc(-c6cccc7c6sc6c8ccccc8ccc76)cc5)c5ccc6c(c5)oc5ccccc56)cc4)cccc32)cc1]

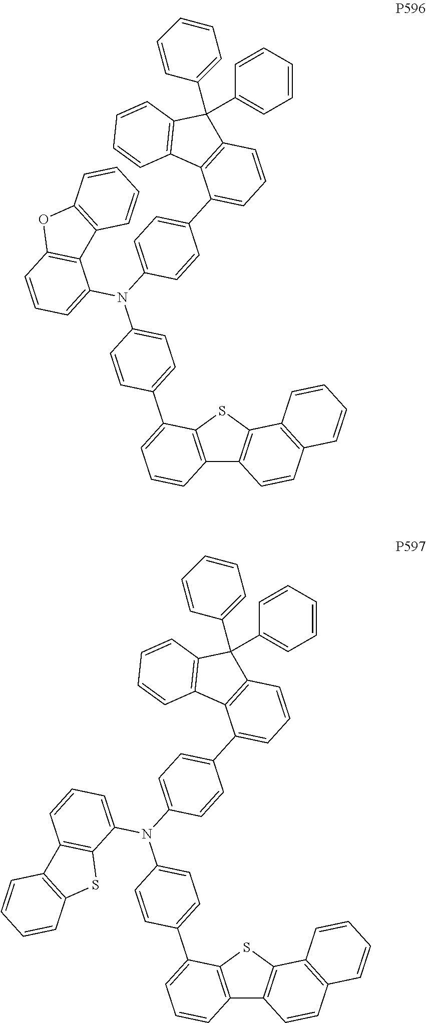 [SMILES: c1ccc(C2(c3ccccc3)c3ccccc3-c3c(-c4ccc(N(c5ccc(-c6cccc7c6sc6c8ccccc8ccc76)cc5)c5cccc6oc7ccccc7c56)cc4)cccc32)cc1 ; c1ccc(C2(c3ccccc3)c3ccccc3-c3c(-c4ccc(N(c5ccc(-c6cccc7c6sc6c8ccccc8ccc76)cc5)c5cccc6c5sc5ccccc56)cc4)cccc32)cc1]

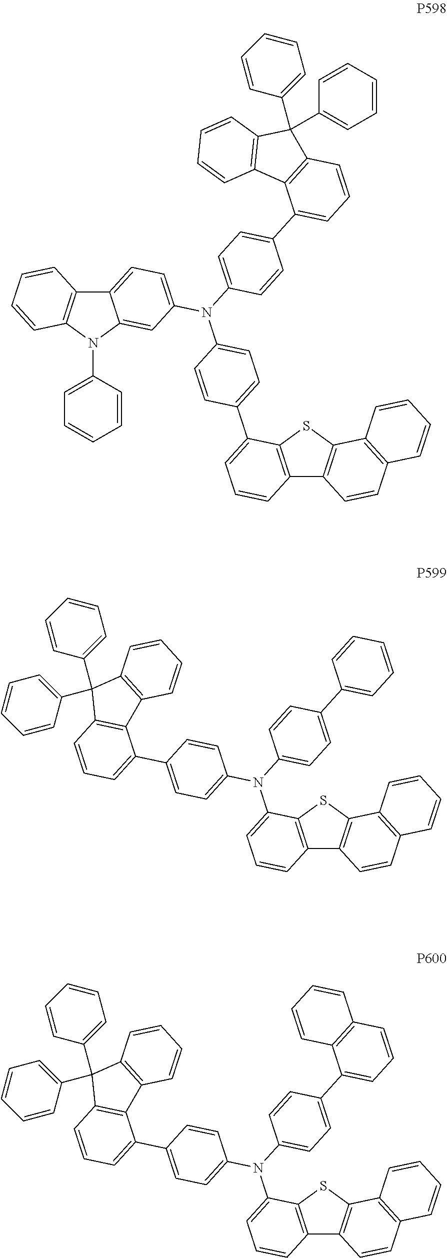 [SMILES: c1ccc(-n2c3ccccc3c3ccc(N(c4ccc(-c5cccc6c5-c5ccccc5C6(c5ccccc5)c5ccccc5)cc4)c4ccc(-c5cccc6c5sc5c7ccccc7ccc65)cc4)cc32)cc1 ; c1ccc(-c2ccc(N(c3ccc(-c4cccc5c4-c4ccccc4C5(c4ccccc4)c4ccccc4)cc3)c3cccc4c3sc3c5ccccc5ccc43)cc2)cc1 ; c1ccc(C2(c3ccccc3)c3ccccc3-c3c(-c4ccc(N(c5ccc(-c6cccc7ccccc67)cc5)c5cccc6c5sc5c7ccccc7ccc65)cc4)cccc32)cc1]

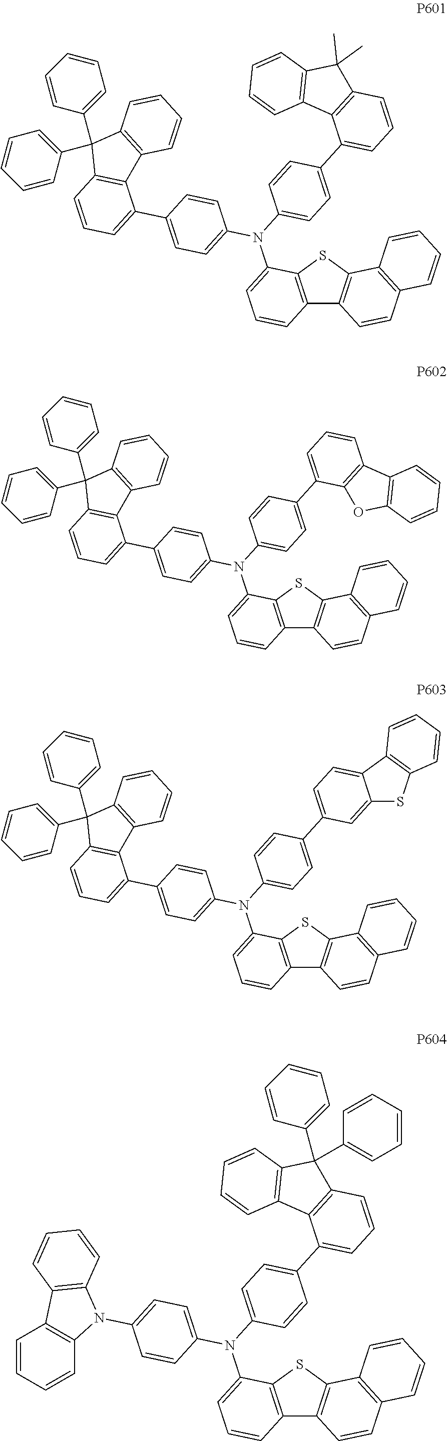 [SMILES: CC1(C)c2ccccc2-c2c(-c3ccc(N(c4ccc(-c5cccc6c5-c5ccccc5C6(c5ccccc5)c5ccccc5)cc4)c4cccc5c4sc4c6ccccc6ccc54)cc3)cccc21 ; c1ccc(C2(c3ccccc3)c3ccccc3-c3c(-c4ccc(N(c5ccc(-c6cccc7c6oc6ccccc67)cc5)c5cccc6c5sc5c7ccccc7ccc65)cc4)cccc32)cc1 ; c1ccc(C2(c3ccccc3)c3ccccc3-c3c(-c4ccc(N(c5ccc(-c6ccc7c(c6)sc6ccccc67)cc5)c5cccc6c5sc5c7ccccc7ccc65)cc4)cccc32)cc1 ; c1ccc(C2(c3ccccc3)c3ccccc3-c3c(-c4ccc(N(c5ccc(-n6c7ccccc7c7ccccc76)cc5)c5cccc6c5sc5c7ccccc7ccc65)cc4)cccc32)cc1]

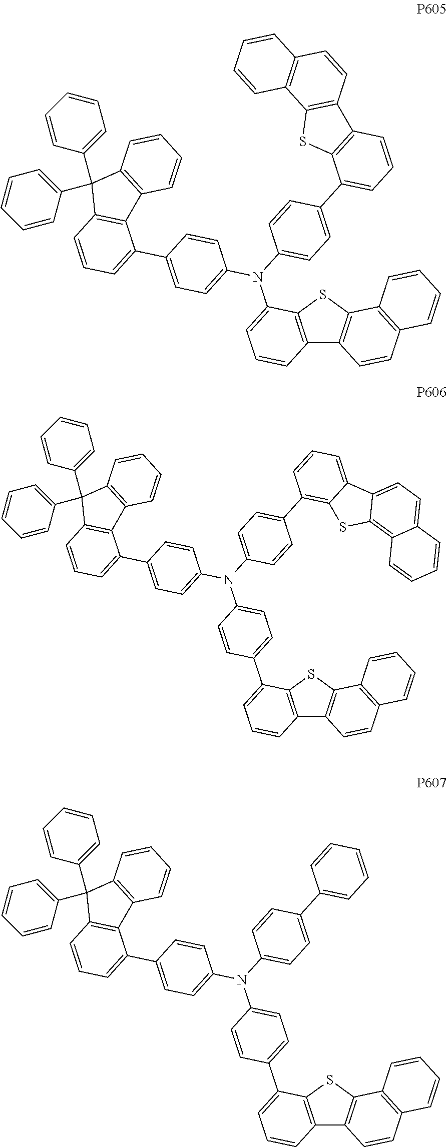 [SMILES: c1ccc(C2(c3ccccc3)c3ccccc3-c3c(-c4ccc(N(c5ccc(-c6cccc7c6sc6c8ccccc8ccc76)cc5)c5cccc6c5sc5c7ccccc7ccc65)cc4)cccc32)cc1 ; c1ccc(C2(c3ccccc3)c3ccccc3-c3c(-c4ccc(N(c5ccc(-c6cccc7c6sc6c8ccccc8ccc76)cc5)c5ccc(-c6cccc7c6sc6c8ccccc8ccc76)cc5)cc4)cccc32)cc1 ; c1ccc(-c2ccc(N(c3ccc(-c4cccc5c4-c4ccccc4C5(c4ccccc4)c4ccccc4)cc3)c3ccc(-c4cccc5c4sc4c6ccccc6ccc54)cc3)cc2)cc1]

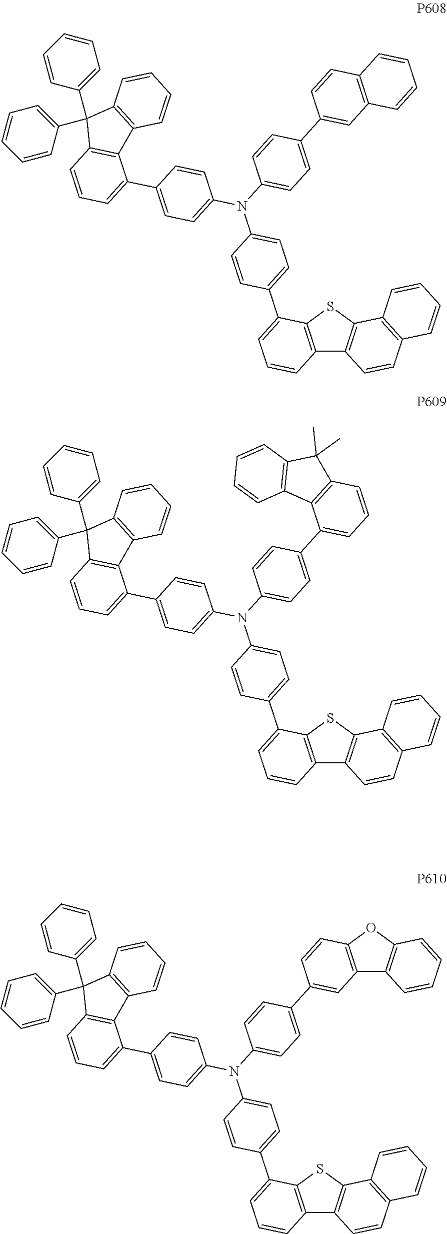 [SMILES: c1ccc(C2(c3ccccc3)c3ccccc3-c3c(-c4ccc(N(c5ccc(-c6ccc7ccccc7c6)cc5)c5ccc(-c6cccc7c6sc6c8ccccc8ccc76)cc5)cc4)cccc32)cc1 ; CC1(C)c2ccccc2-c2c(-c3ccc(N(c4ccc(-c5cccc6c5-c5ccccc5C6(c5ccccc5)c5ccccc5)cc4)c4ccc(-c5cccc6c5sc5c7ccccc7ccc65)cc4)cc3)cccc21 ; c1ccc(C2(c3ccccc3)c3ccccc3-c3c(-c4ccc(N(c5ccc(-c6ccc7oc8ccccc8c7c6)cc5)c5ccc(-c6cccc7c6sc6c8ccccc8ccc76)cc5)cc4)cccc32)cc1]

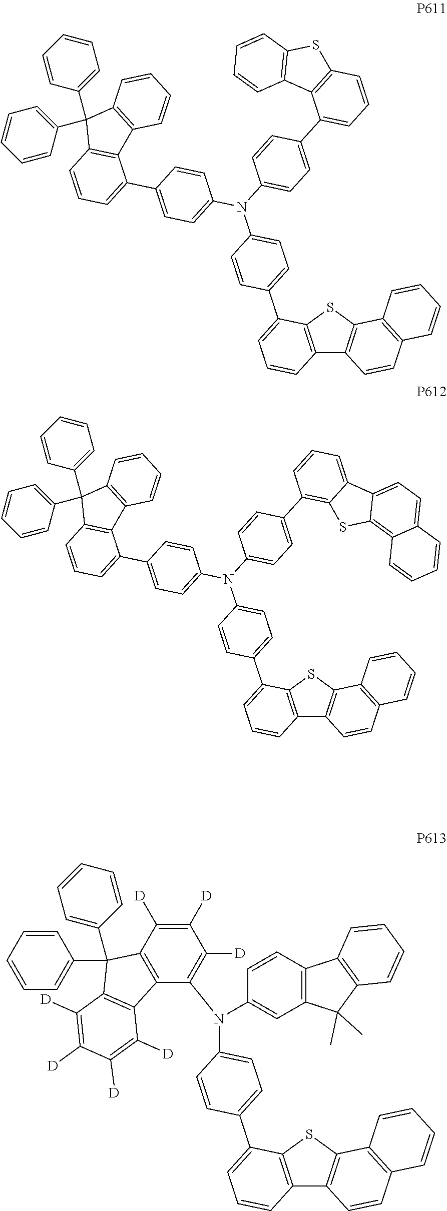 [SMILES: c1ccc(C2(c3ccccc3)c3ccccc3-c3c(-c4ccc(N(c5ccc(-c6cccc7c6sc6c8ccccc8ccc76)cc5)c5ccc(-c6cccc7sc8ccccc8c67)cc5)cc4)cccc32)cc1 ; c1ccc(C2(c3ccccc3)c3ccccc3-c3c(-c4ccc(N(c5ccc(-c6cccc7c6sc6c8ccccc8ccc76)cc5)c5ccc(-c6cccc7c6sc6c8ccccc8ccc76)cc5)cc4)cccc32)cc1 ; [2H]c1c([2H])c([2H])c2c(c1[2H])-c1c(N(c3ccc(-c4cccc5c4sc4c6ccccc6ccc54)cc3)c3ccc4c(c3)C(C)(C)c3ccccc3-4)c([2H])c([2H])c([2H])c1C2(c1ccccc1)c1ccccc1]

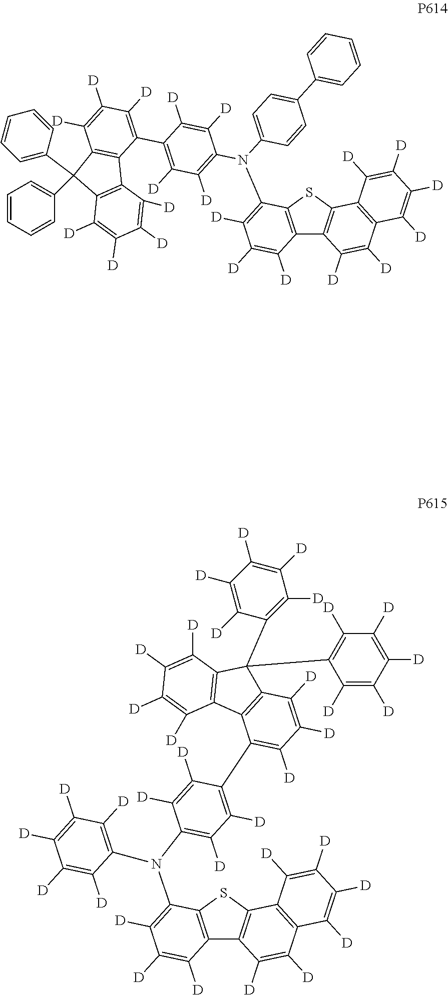

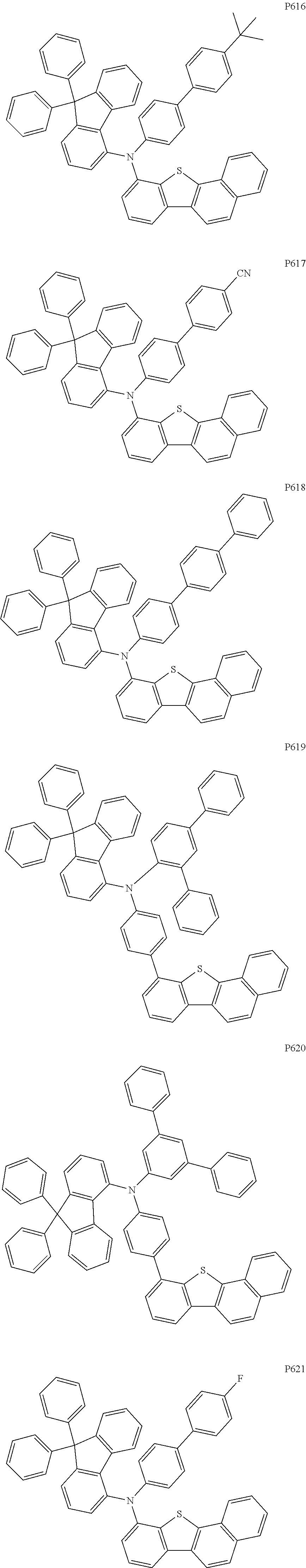

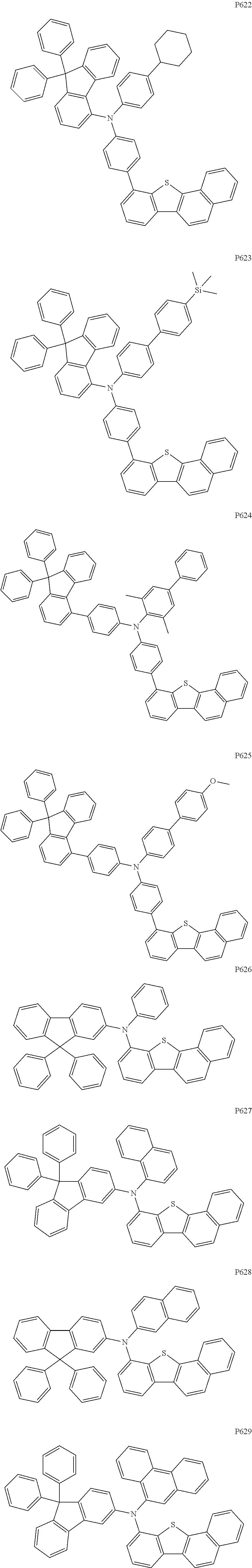

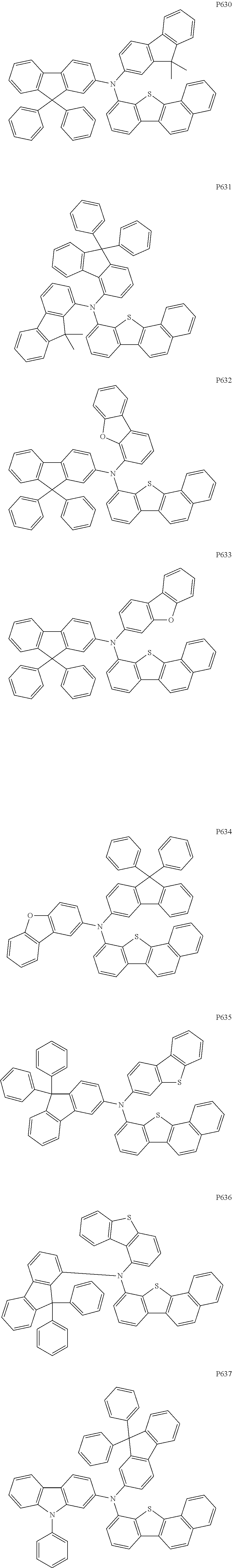

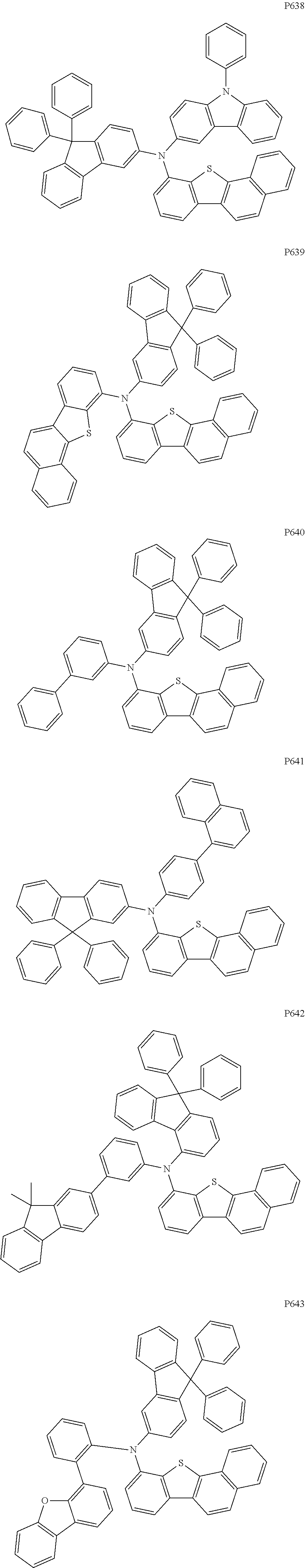

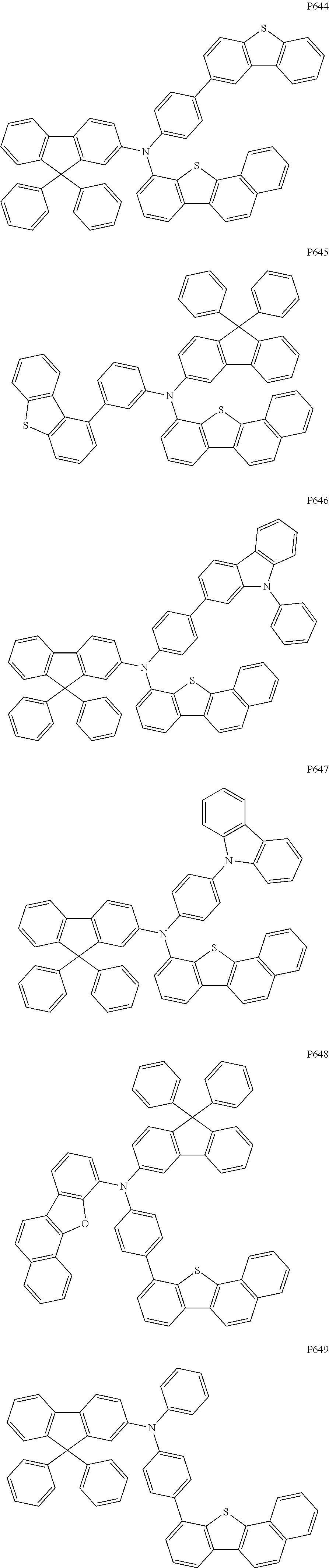

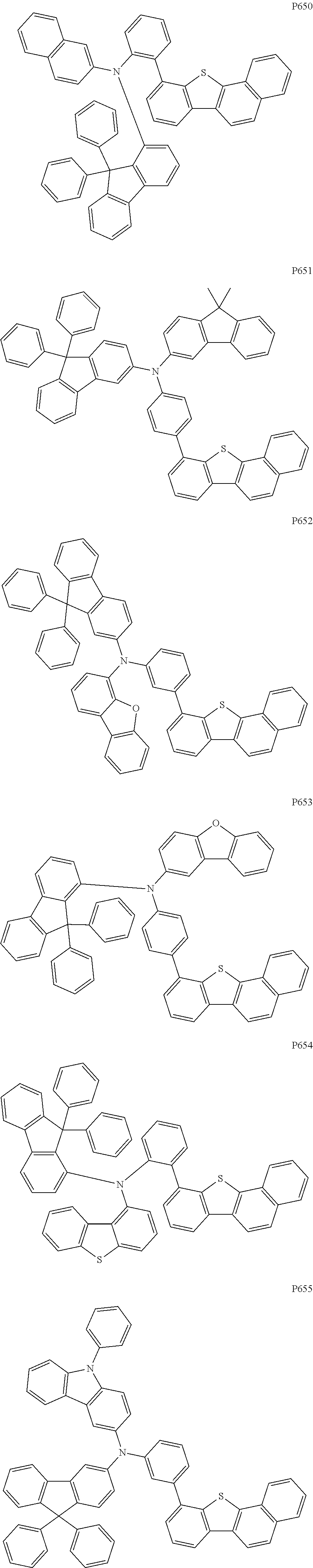

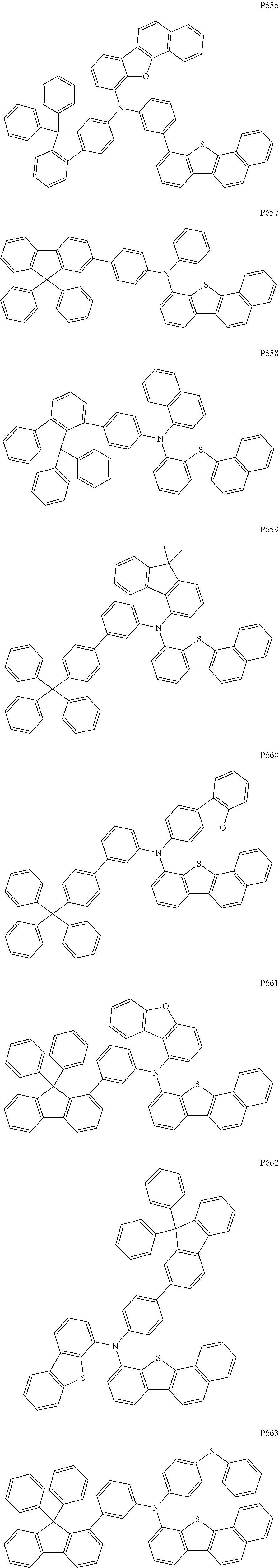

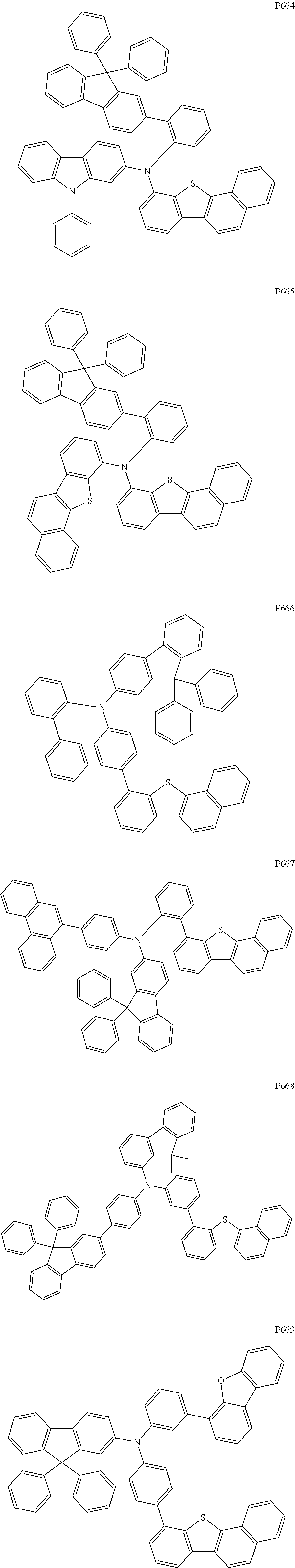

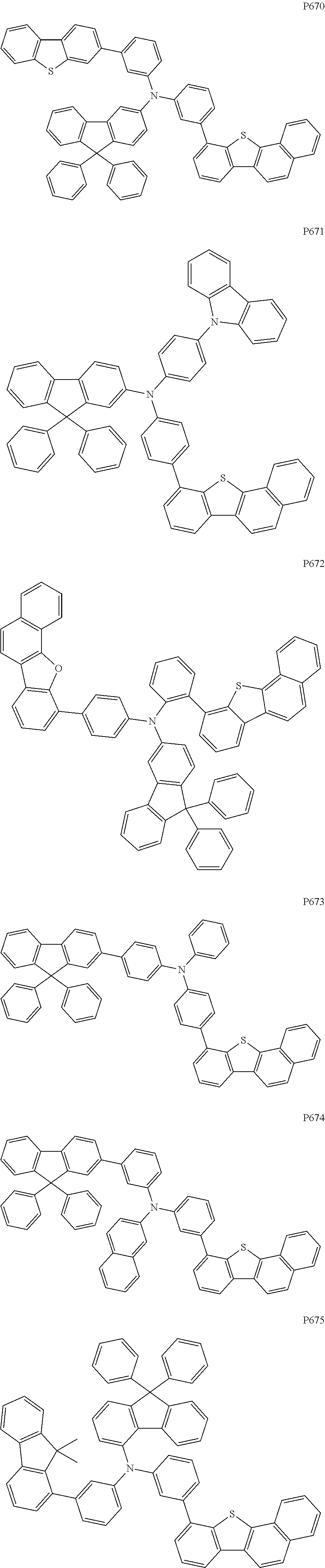

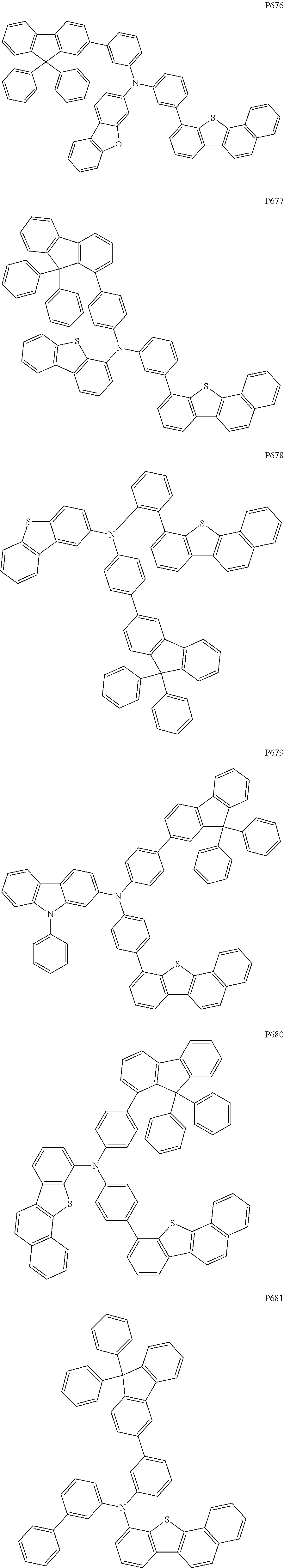

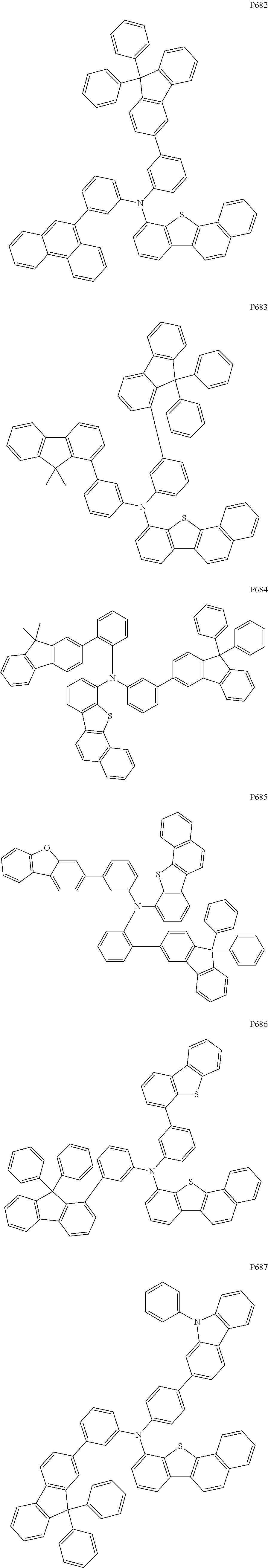

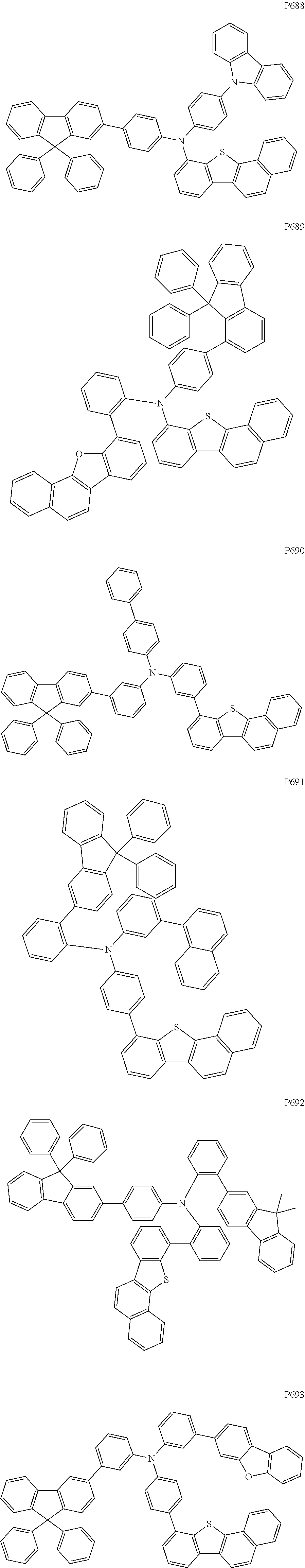

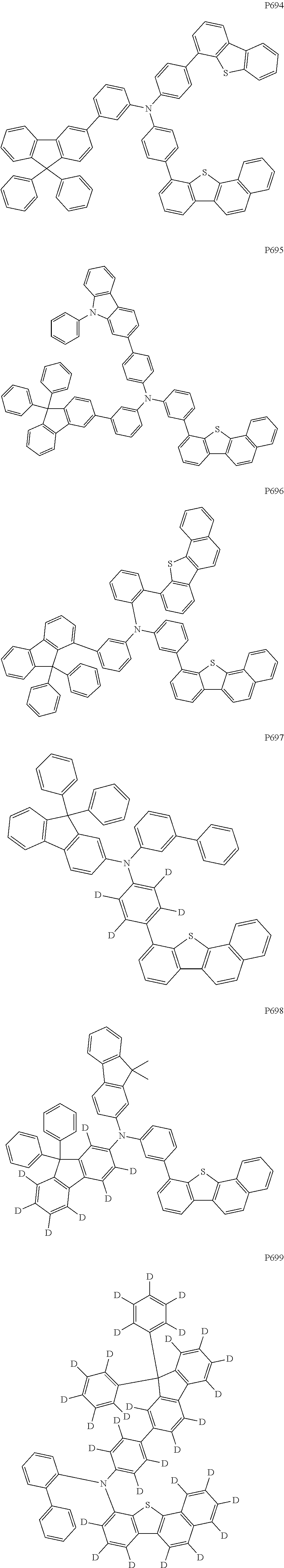

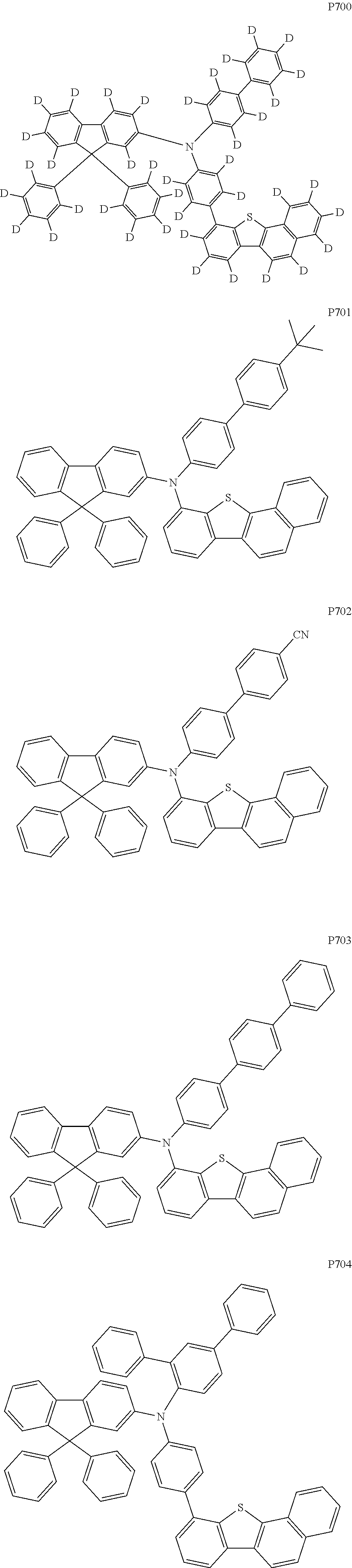

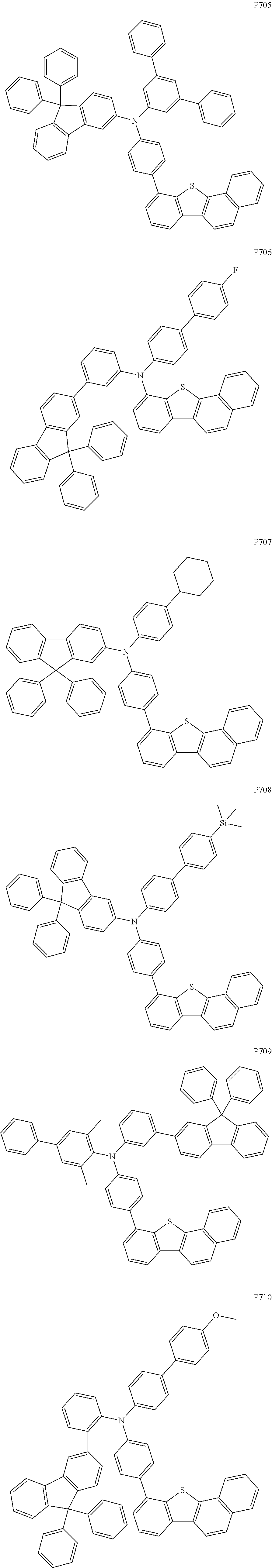

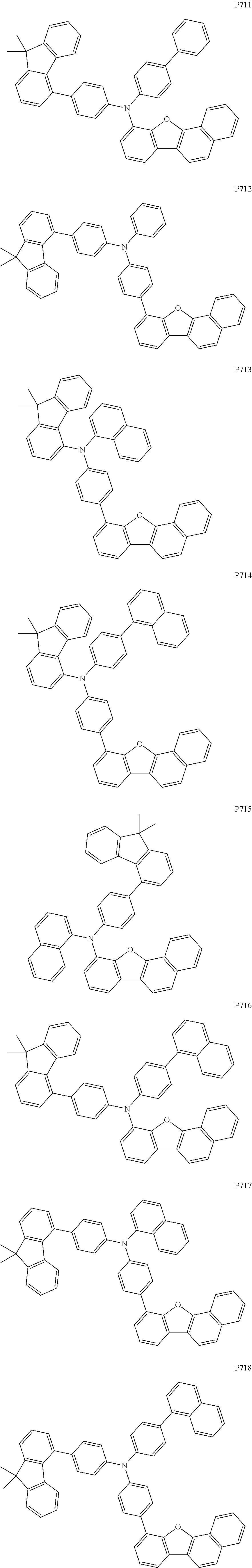

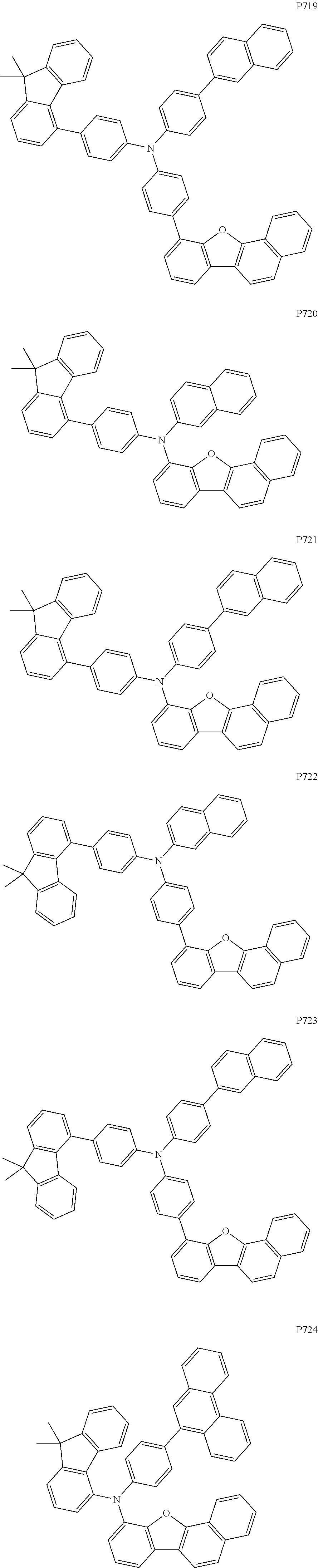

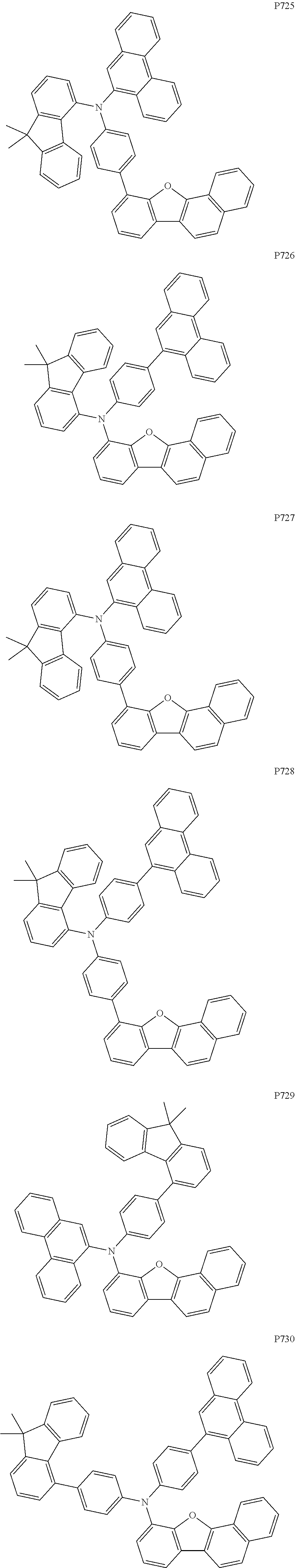

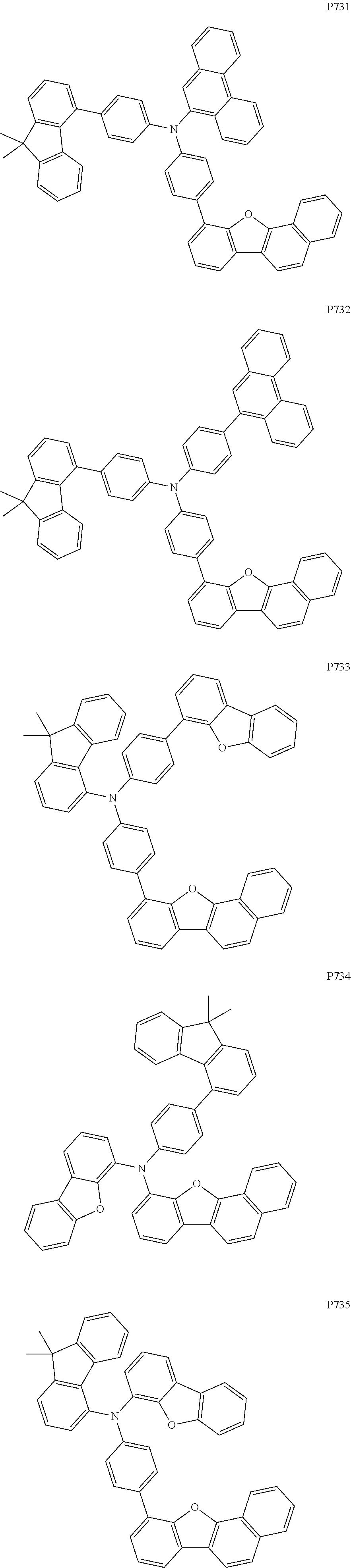 [SMILES: CC1(C)c2ccccc2-c2c(-c3ccc(N(c4ccc(-c5cccc6c5oc5c7ccccc7ccc65)cc4)c4cc5ccccc5c5ccccc45)cc3)cccc21 ; CC1(C)c2ccccc2-c2c(-c3ccc(N(c4ccc(-c5cc6ccccc6c6ccccc56)cc4)c4ccc(-c5cccc6c5oc5c7ccccc7ccc65)cc4)cc3)cccc21 ; CC1(C)c2ccccc2-c2c(N(c3ccc(-c4cccc5c4oc4ccccc45)cc3)c3ccc(-c4cccc5c4oc4c6ccccc6ccc54)cc3)cccc21 ; CC1(C)c2ccccc2-c2c(-c3ccc(N(c4cccc5c4oc4ccccc45)c4cccc5c4oc4c6ccccc6ccc54)cc3)cccc21 ; CC1(C)c2ccccc2-c2c(N(c3ccc(-c4cccc5c4oc4c6ccccc6ccc54)cc3)c3cccc4c3oc3ccccc34)cccc21]

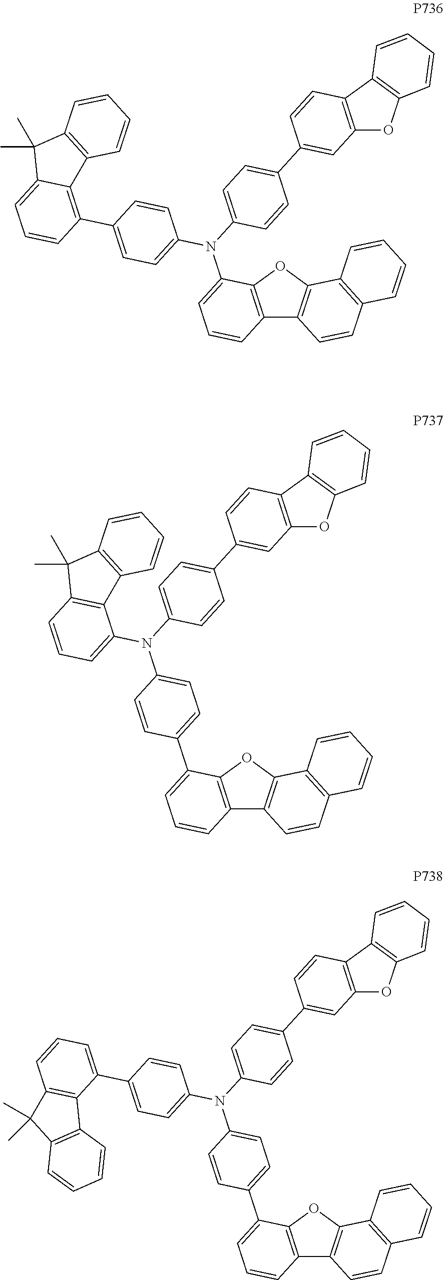 [SMILES: CC1(C)c2ccccc2-c2c(-c3ccc(N(c4ccc(-c5ccc6c(c5)oc5ccccc56)cc4)c4cccc5c4oc4c6ccccc6ccc54)cc3)cccc21 ; CC1(C)c2ccccc2-c2c(N(c3ccc(-c4ccc5c(c4)oc4ccccc45)cc3)c3ccc(-c4cccc5c4oc4c6ccccc6ccc54)cc3)cccc21 ; CC1(C)c2ccccc2-c2c(-c3ccc(N(c4ccc(-c5ccc6c(c5)oc5ccccc56)cc4)c4ccc(-c5cccc6c5oc5c7ccccc7ccc65)cc4)cc3)cccc21]

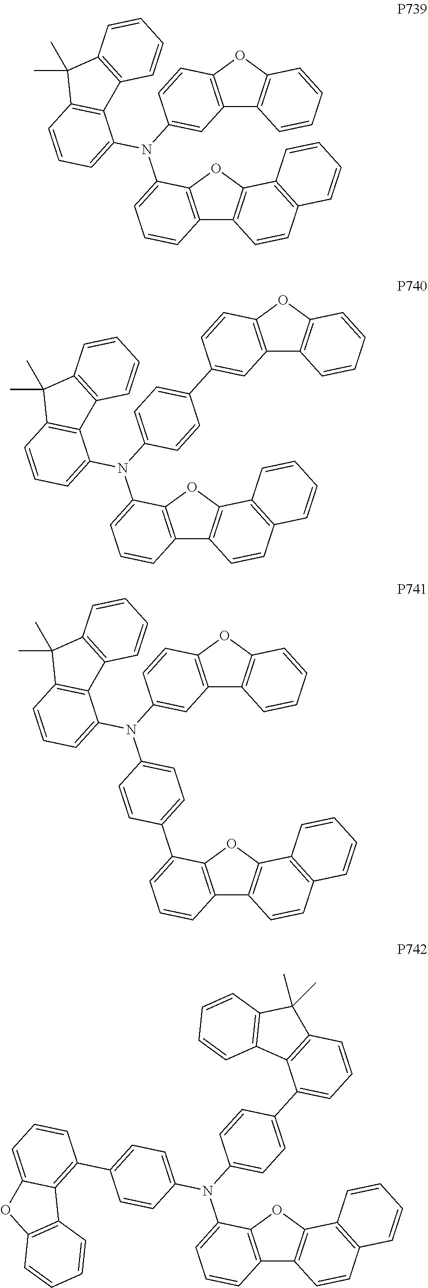

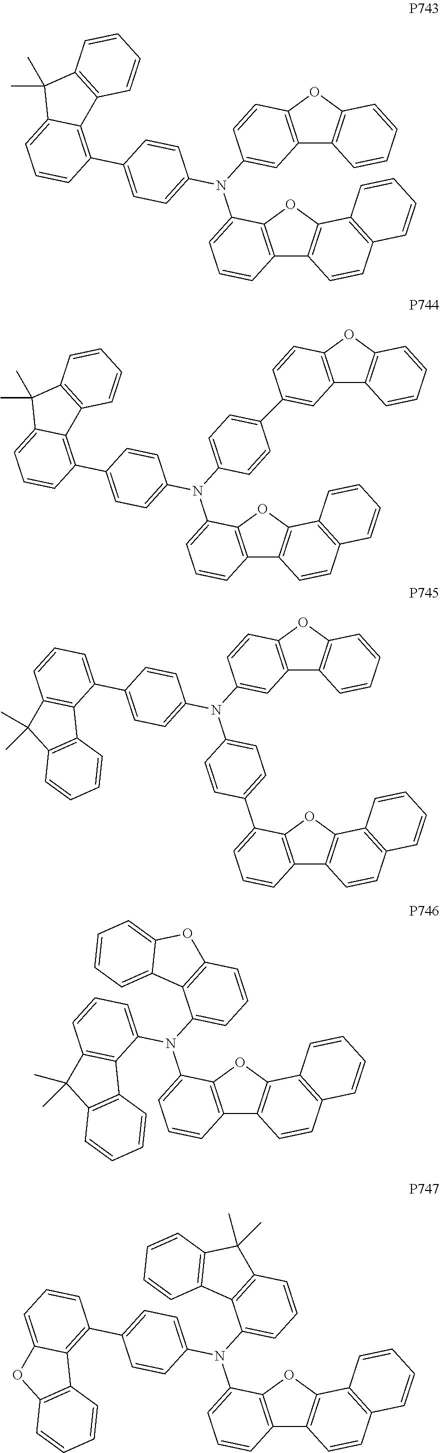

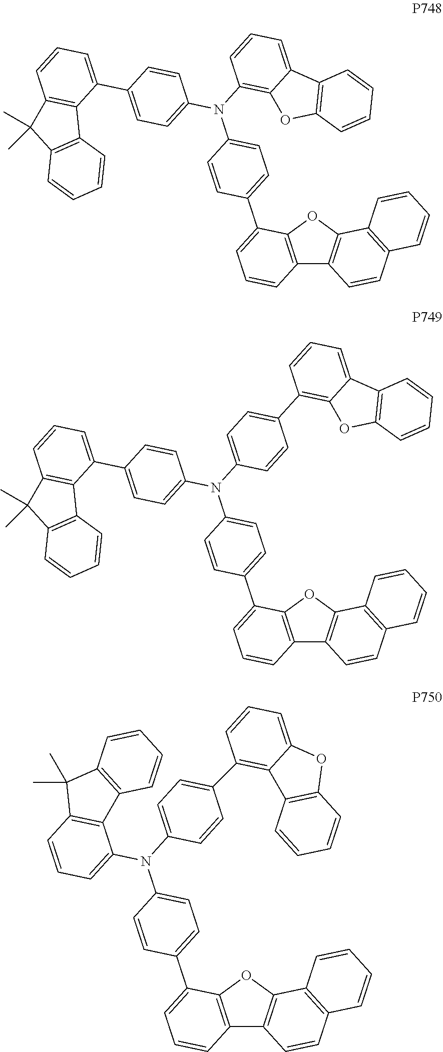 [SMILES: CC1(C)c2ccccc2-c2c(-c3ccc(N(c4ccc(-c5cccc6c5oc5c7ccccc7ccc65)cc4)c4cccc5c4oc4ccccc45)cc3)cccc21 ; CC1(C)c2ccccc2-c2c(-c3ccc(N(c4ccc(-c5cccc6c5oc5ccccc56)cc4)c4ccc(-c5cccc6c5oc5c7ccccc7ccc65)cc4)cc3)cccc21 ; CC1(C)c2ccccc2-c2c(N(c3ccc(-c4cccc5c4oc4c6ccccc6ccc54)cc3)c3ccc(-c4cccc5oc6ccccc6c45)cc3)cccc21]

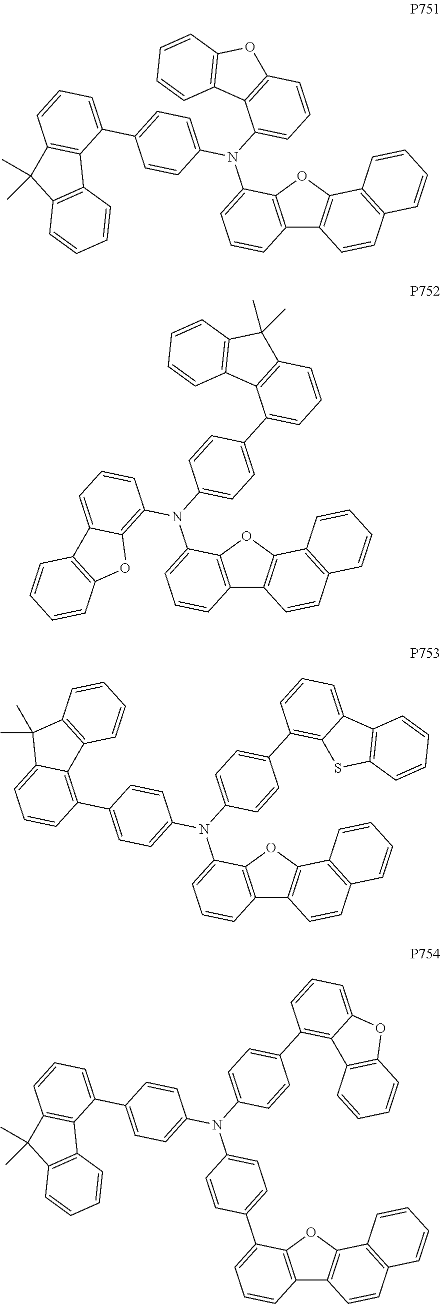 [SMILES: CC1(C)c2ccccc2-c2c(-c3ccc(N(c4cccc5c4oc4c6ccccc6ccc54)c4cccc5oc6ccccc6c45)cc3)cccc21 ; CC1(C)c2ccccc2-c2c(-c3ccc(N(c4cccc5c4oc4ccccc45)c4cccc5c4oc4c6ccccc6ccc54)cc3)cccc21 ; CC1(C)c2ccccc2-c2c(-c3ccc(N(c4ccc(-c5cccc6c5sc5ccccc56)cc4)c4cccc5c4oc4c6ccccc6ccc54)cc3)cccc21 ; CC1(C)c2ccccc2-c2c(-c3ccc(N(c4ccc(-c5cccc6c5oc5c7ccccc7ccc65)cc4)c4ccc(-c5cccc6oc7ccccc7c56)cc4)cc3)cccc21]

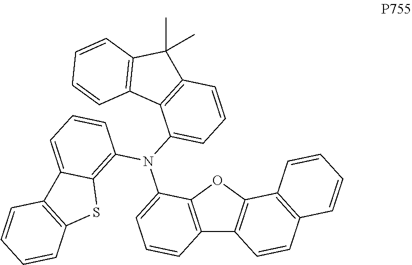 [SMILES: CC1(C)c2ccccc2-c2c(N(c3cccc4c3oc3c5ccccc5ccc43)c3cccc4c3sc3ccccc34)cccc21]

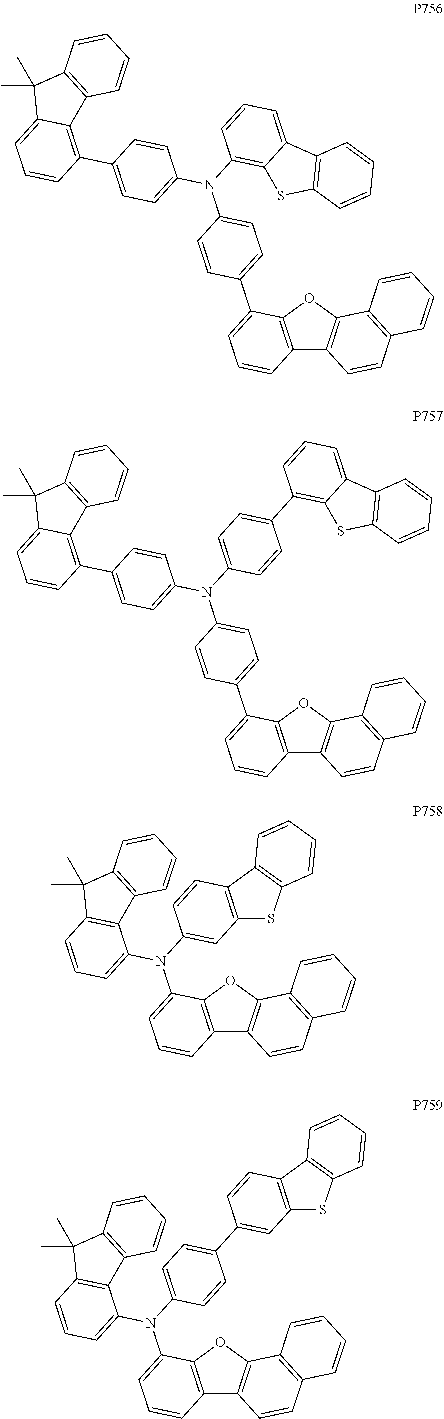 [SMILES: CC1(C)c2ccccc2-c2c(-c3ccc(N(c4ccc(-c5cccc6c5oc5c7ccccc7ccc65)cc4)c4cccc5c4sc4ccccc45)cc3)cccc21 ; CC1(C)c2ccccc2-c2c(-c3ccc(N(c4ccc(-c5cccc6c5oc5c7ccccc7ccc65)cc4)c4ccc(-c5cccc6c5sc5ccccc56)cc4)cc3)cccc21 ; CC1(C)c2ccccc2-c2c(N(c3ccc4c(c3)sc3ccccc34)c3cccc4c3oc3c5ccccc5ccc43)cccc21 ; CC1(C)c2ccccc2-c2c(N(c3ccc(-c4ccc5c(c4)sc4ccccc45)cc3)c3cccc4c3oc3c5ccccc5ccc43)cccc21]

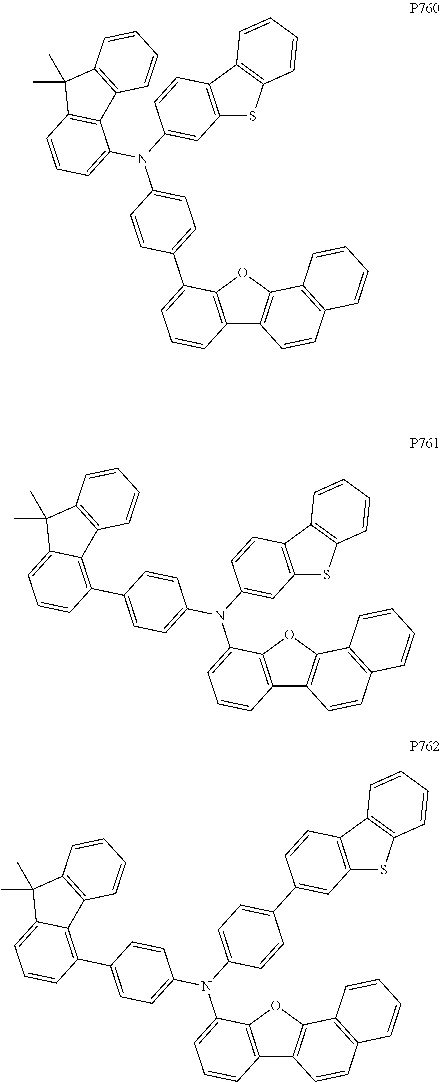 [SMILES: CC1(C)c2ccccc2-c2c(N(c3ccc(-c4cccc5c4oc4c6ccccc6ccc54)cc3)c3ccc4c(c3)sc3ccccc34)cccc21 ; CC1(C)c2ccccc2-c2c(-c3ccc(N(c4ccc5c(c4)sc4ccccc45)c4cccc5c4oc4c6ccccc6ccc54)cc3)cccc21 ; CC1(C)c2ccccc2-c2c(-c3ccc(N(c4ccc(-c5ccc6c(c5)sc5ccccc56)cc4)c4cccc5c4oc4c6ccccc6ccc54)cc3)cccc21]

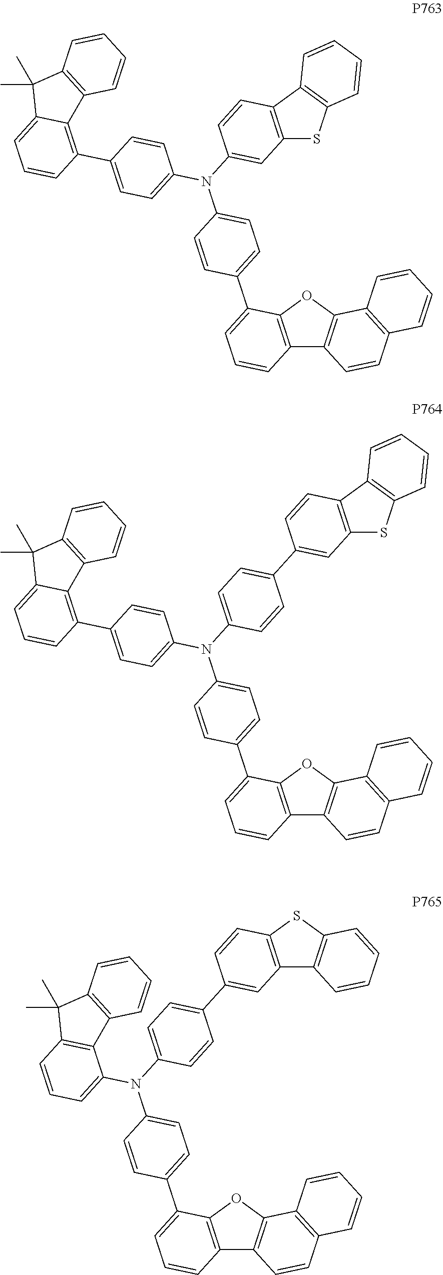 [SMILES: CC1(C)c2ccccc2-c2c(-c3ccc(N(c4ccc(-c5cccc6c5oc5c7ccccc7ccc65)cc4)c4ccc5c(c4)sc4ccccc45)cc3)cccc21 ; CC1(C)c2ccccc2-c2c(-c3ccc(N(c4ccc(-c5ccc6c(c5)sc5ccccc56)cc4)c4ccc(-c5cccc6c5oc5c7ccccc7ccc65)cc4)cc3)cccc21 ; CC1(C)c2ccccc2-c2c(N(c3ccc(-c4ccc5sc6ccccc6c5c4)cc3)c3ccc(-c4cccc5c4oc4c6ccccc6ccc54)cc3)cccc21]

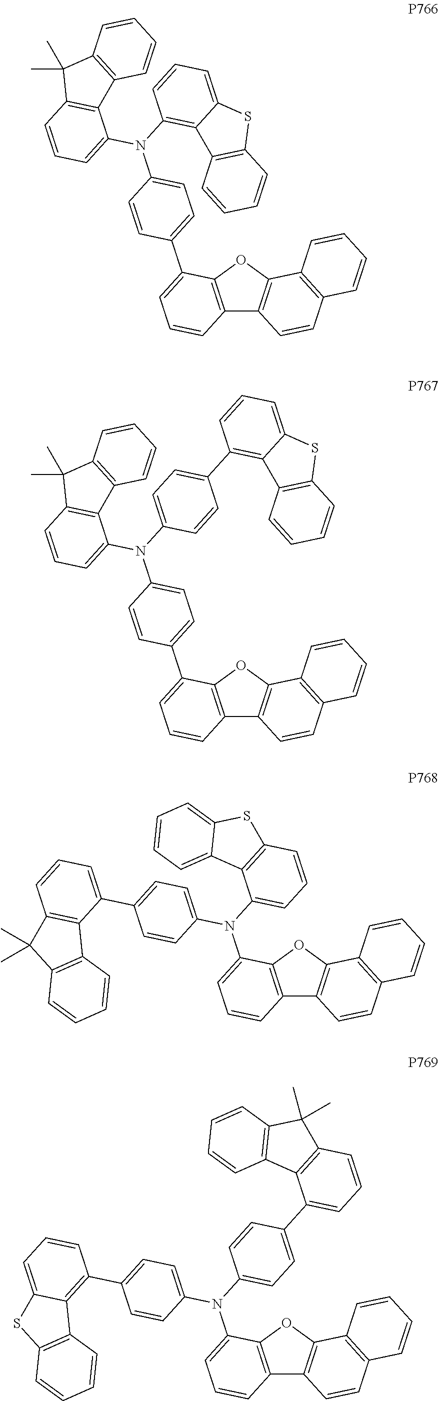 [SMILES: CC1(C)c2ccccc2-c2c(N(c3ccc(-c4cccc5c4oc4c6ccccc6ccc54)cc3)c3cccc4sc5ccccc5c34)cccc21 ; CC1(C)c2ccccc2-c2c(N(c3ccc(-c4cccc5c4oc4c6ccccc6ccc54)cc3)c3ccc(-c4cccc5sc6ccccc6c45)cc3)cccc21 ; CC1(C)c2ccccc2-c2c(-c3ccc(N(c4cccc5c4oc4c6ccccc6ccc54)c4cccc5sc6ccccc6c45)cc3)cccc21 ; CC1(C)c2ccccc2-c2c(-c3ccc(N(c4ccc(-c5cccc6sc7ccccc7c56)cc4)c4cccc5c4oc4c6ccccc6ccc54)cc3)cccc21]

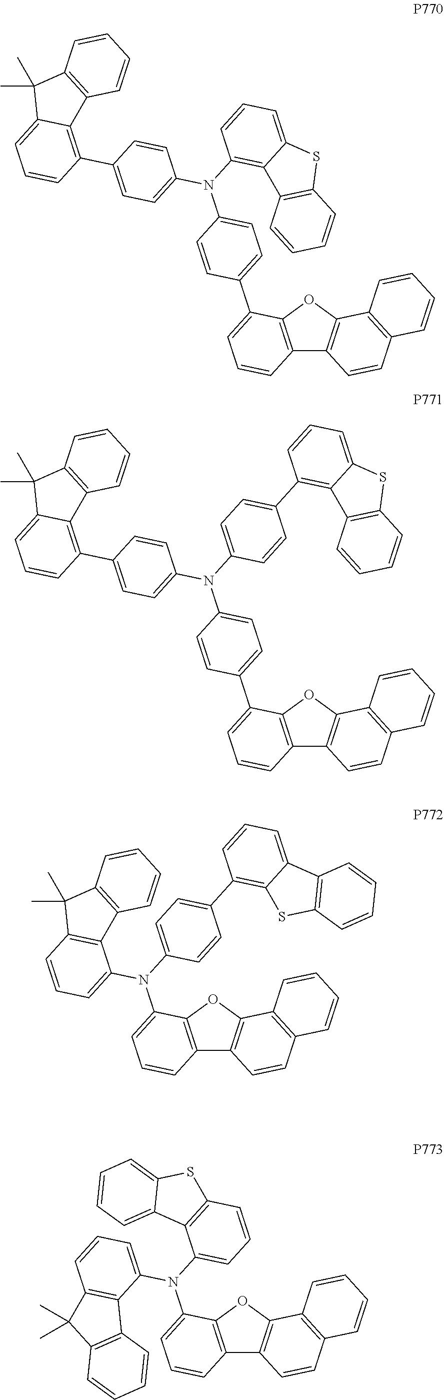 [SMILES: CC1(C)c2ccccc2-c2c(-c3ccc(N(c4ccc(-c5cccc6c5oc5c7ccccc7ccc65)cc4)c4cccc5sc6ccccc6c45)cc3)cccc21 ; CC1(C)c2ccccc2-c2c(-c3ccc(N(c4ccc(-c5cccc6c5oc5c7ccccc7ccc65)cc4)c4ccc(-c5cccc6sc7ccccc7c56)cc4)cc3)cccc21 ; CC1(C)c2ccccc2-c2c(N(c3ccc(-c4cccc5c4sc4ccccc45)cc3)c3cccc4c3oc3c5ccccc5ccc43)cccc21 ; CC1(C)c2ccccc2-c2c(N(c3cccc4c3oc3c5ccccc5ccc43)c3cccc4sc5ccccc5c34)cccc21]

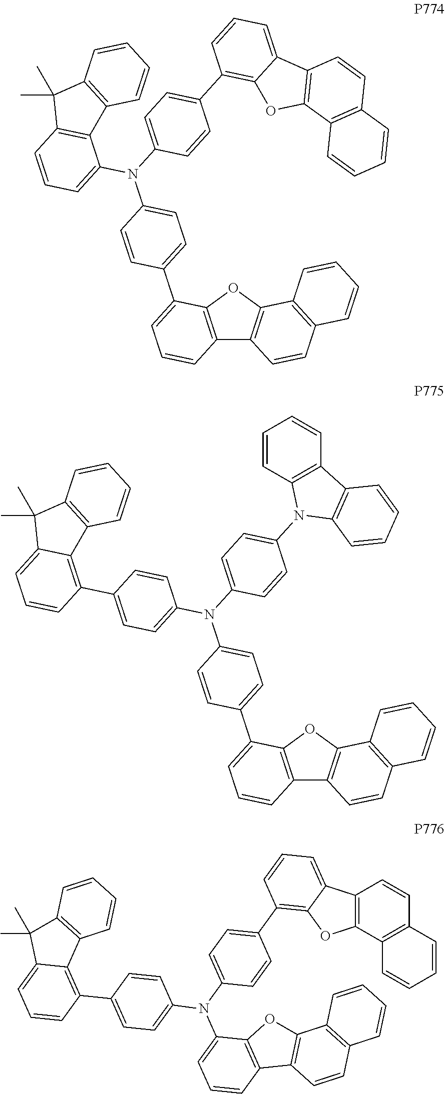 [SMILES: CC1(C)c2ccccc2-c2c(N(c3ccc(-c4cccc5c4oc4c6ccccc6ccc54)cc3)c3ccc(-c4cccc5c4oc4c6ccccc6ccc54)cc3)cccc21 ; CC1(C)c2ccccc2-c2c(-c3ccc(N(c4ccc(-c5cccc6c5oc5c7ccccc7ccc65)cc4)c4ccc(-n5c6ccccc6c6ccccc65)cc4)cc3)cccc21 ; CC1(C)c2ccccc2-c2c(-c3ccc(N(c4ccc(-c5cccc6c5oc5c7ccccc7ccc65)cc4)c4cccc5c4oc4c6ccccc6ccc54)cc3)cccc21]

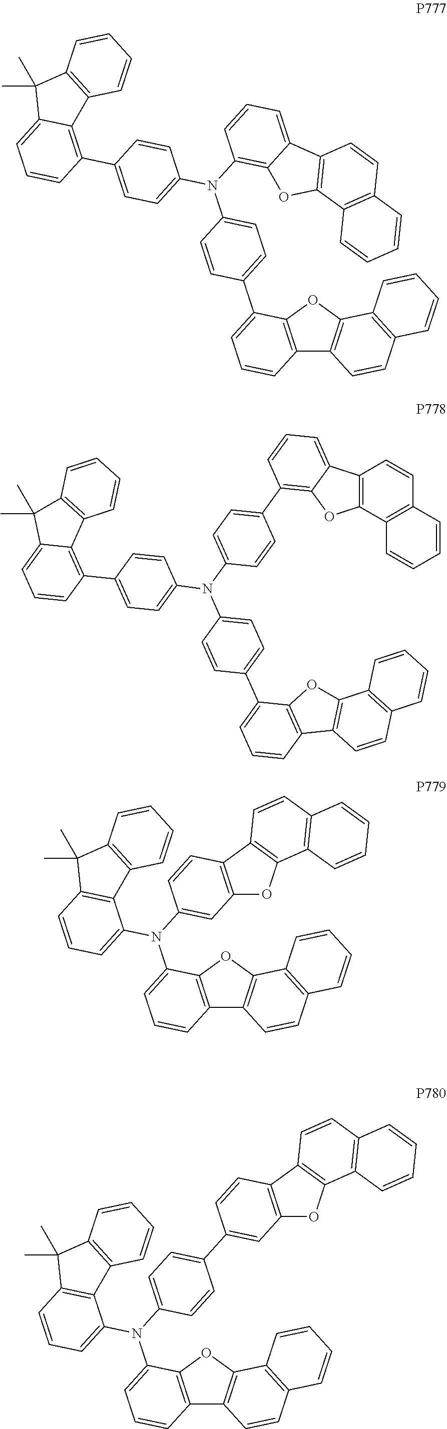

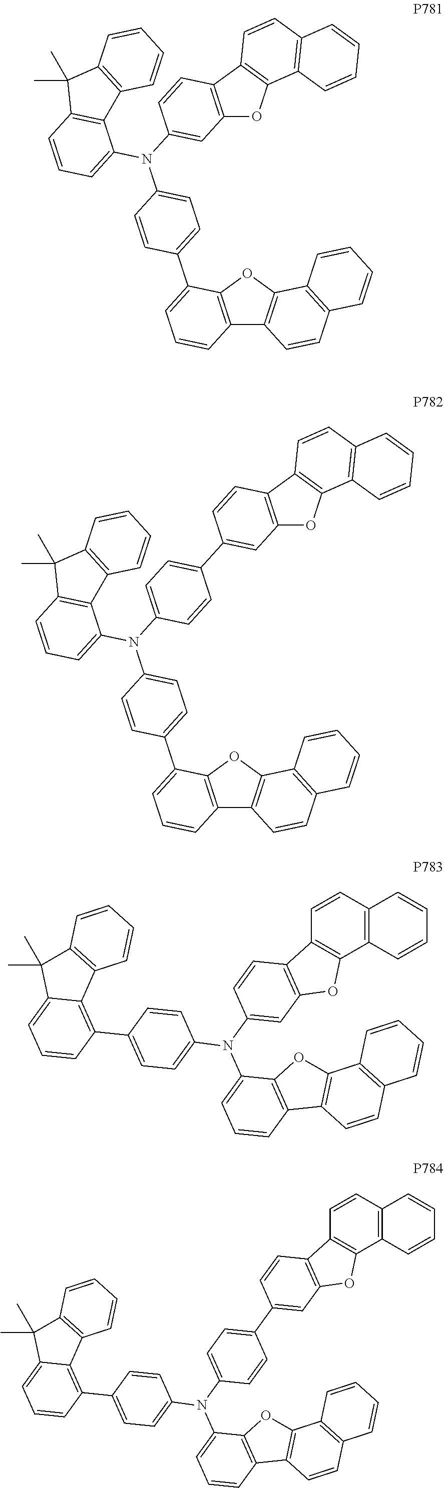 [SMILES: CC1(C)c2ccccc2-c2c(N(c3ccc(-c4cccc5c4oc4c6ccccc6ccc54)cc3)c3ccc4c(c3)oc3c5ccccc5ccc43)cccc21 ; CC1(C)c2ccccc2-c2c(N(c3ccc(-c4ccc5c(c4)oc4c6ccccc6ccc54)cc3)c3ccc(-c4cccc5c4oc4c6ccccc6ccc54)cc3)cccc21 ; CC1(C)c2ccccc2-c2c(-c3ccc(N(c4ccc5c(c4)oc4c6ccccc6ccc54)c4cccc5c4oc4c6ccccc6ccc54)cc3)cccc21 ; CC1(C)c2ccccc2-c2c(-c3ccc(N(c4ccc(-c5ccc6c(c5)oc5c7ccccc7ccc65)cc4)c4cccc5c4oc4c6ccccc6ccc54)cc3)cccc21]

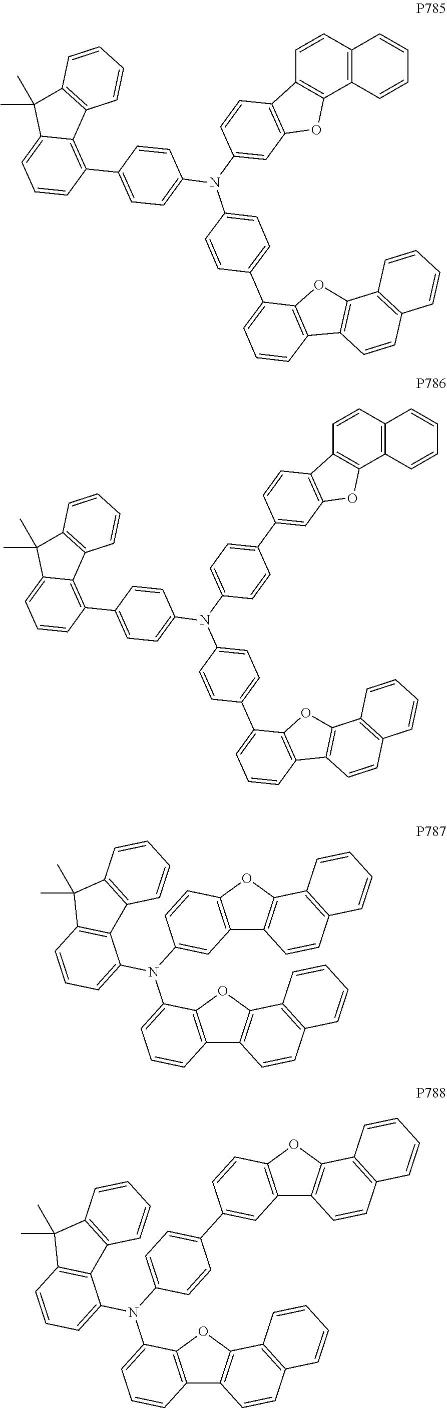 [SMILES: CC1(C)c2ccccc2-c2c(-c3ccc(N(c4ccc(-c5cccc6c5oc5c7ccccc7ccc65)cc4)c4ccc5c(c4)oc4c6ccccc6ccc54)cc3)cccc21 ; CC1(C)c2ccccc2-c2c(-c3ccc(N(c4ccc(-c5ccc6c(c5)oc5c7ccccc7ccc65)cc4)c4ccc(-c5cccc6c5oc5c7ccccc7ccc65)cc4)cc3)cccc21 ; CC1(C)c2ccccc2-c2c(N(c3ccc4oc5c6ccccc6ccc5c4c3)c3cccc4c3oc3c5ccccc5ccc43)cccc21 ; CC1(C)c2ccccc2-c2c(N(c3ccc(-c4ccc5oc6c7ccccc7ccc6c5c4)cc3)c3cccc4c3oc3c5ccccc5ccc43)cccc21]

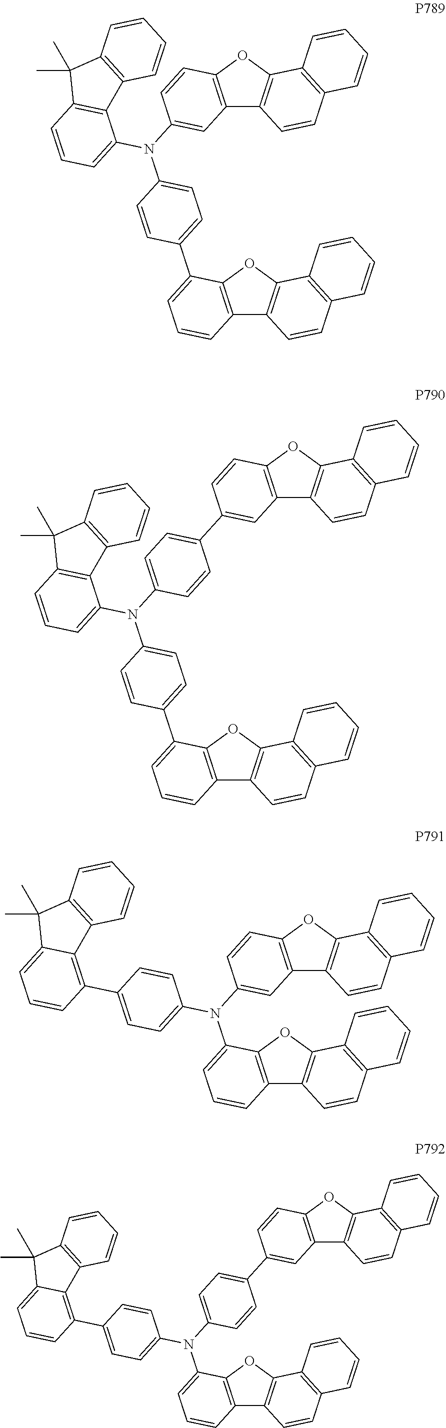 [SMILES: CC1(C)c2ccccc2-c2c(N(c3ccc(-c4cccc5c4oc4c6ccccc6ccc54)cc3)c3ccc4oc5c6ccccc6ccc5c4c3)cccc21 ; CC1(C)c2ccccc2-c2c(N(c3ccc(-c4ccc5oc6c7ccccc7ccc6c5c4)cc3)c3ccc(-c4cccc5c4oc4c6ccccc6ccc54)cc3)cccc21 ; CC1(C)c2ccccc2-c2c(-c3ccc(N(c4ccc5oc6c7ccccc7ccc6c5c4)c4cccc5c4oc4c6ccccc6ccc54)cc3)cccc21 ; CC1(C)c2ccccc2-c2c(-c3ccc(N(c4ccc(-c5ccc6oc7c8ccccc8ccc7c6c5)cc4)c4cccc5c4oc4c6ccccc6ccc54)cc3)cccc21]

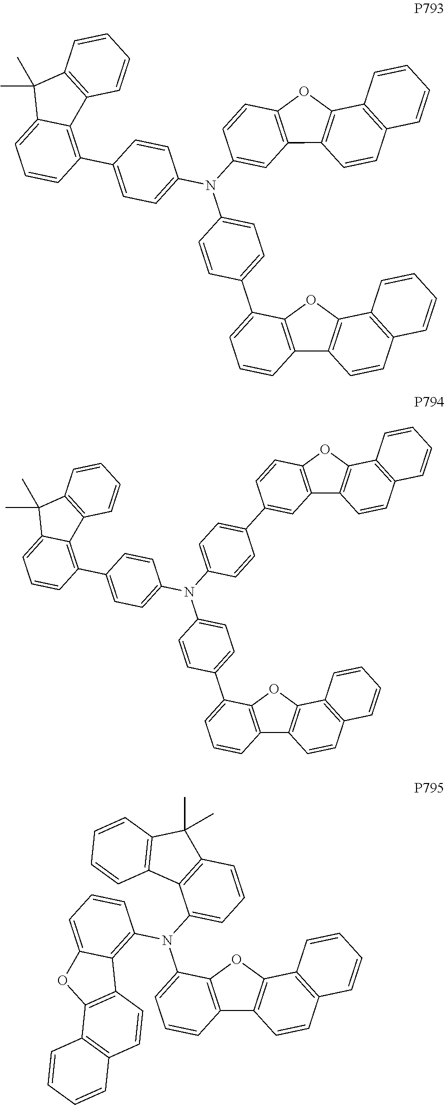 [SMILES: CC1(C)c2ccccc2-c2c(-c3ccc(N(c4ccc(-c5cccc6c5oc5c7ccccc7ccc65)cc4)c4ccc5oc6c7ccccc7ccc6c5c4)cc3)cccc21 ; CC1(C)c2ccccc2-c2c(-c3ccc(N(c4ccc(-c5ccc6oc7c8ccccc8ccc7c6c5)cc4)c4ccc(-c5cccc6c5oc5c7ccccc7ccc65)cc4)cc3)cccc21 ; CC1(C)c2ccccc2-c2c(N(c3cccc4c3oc3c5ccccc5ccc43)c3cccc4oc5c6ccccc6ccc5c34)cccc21]

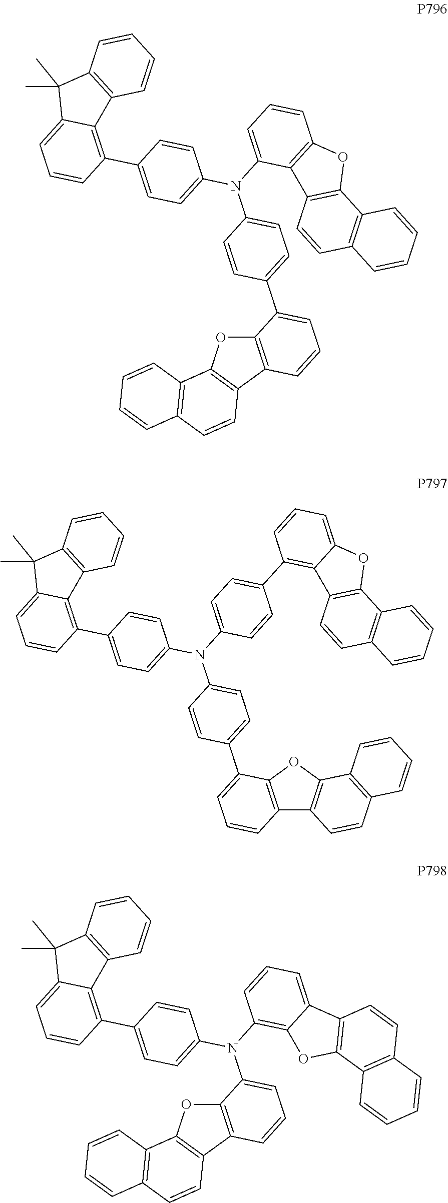 [SMILES: CC1(C)c2ccccc2-c2c(-c3ccc(N(c4ccc(-c5cccc6c5oc5c7ccccc7ccc65)cc4)c4cccc5oc6c7ccccc7ccc6c45)cc3)cccc21 ; CC1(C)c2ccccc2-c2c(-c3ccc(N(c4ccc(-c5cccc6c5oc5c7ccccc7ccc65)cc4)c4ccc(-c5cccc6oc7c8ccccc8ccc7c56)cc4)cc3)cccc21 ; CC1(C)c2ccccc2-c2c(-c3ccc(N(c4cccc5c4oc4c6ccccc6ccc54)c4cccc5c4oc4c6ccccc6ccc54)cc3)cccc21]

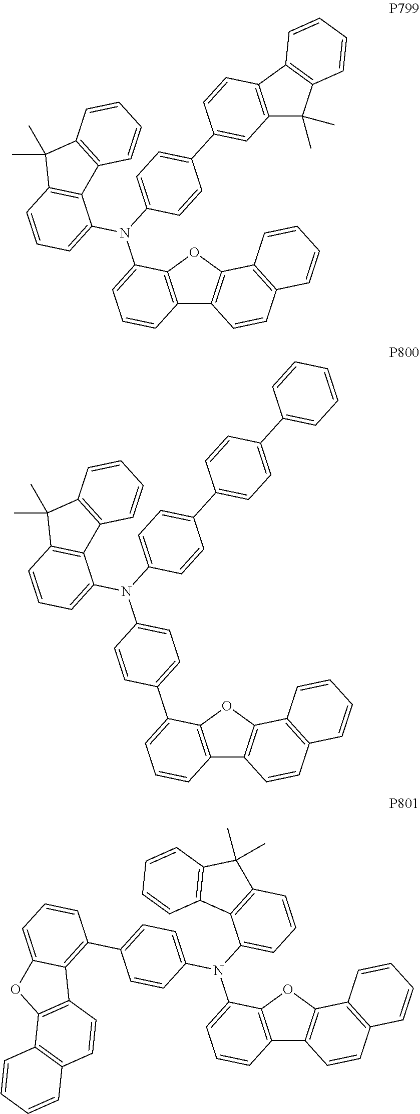 [SMILES: CC1(C)c2ccccc2-c2ccc(-c3ccc(N(c4cccc5c4-c4ccccc4C5(C)C)c4cccc5c4oc4c6ccccc6ccc54)cc3)cc21 ; CC1(C)c2ccccc2-c2c(N(c3ccc(-c4ccc(-c5ccccc5)cc4)cc3)c3ccc(-c4cccc5c4oc4c6ccccc6ccc54)cc3)cccc21 ; CC1(C)c2ccccc2-c2c(N(c3ccc(-c4cccc5oc6c7ccccc7ccc6c45)cc3)c3cccc4c3oc3c5ccccc5ccc43)cccc21]

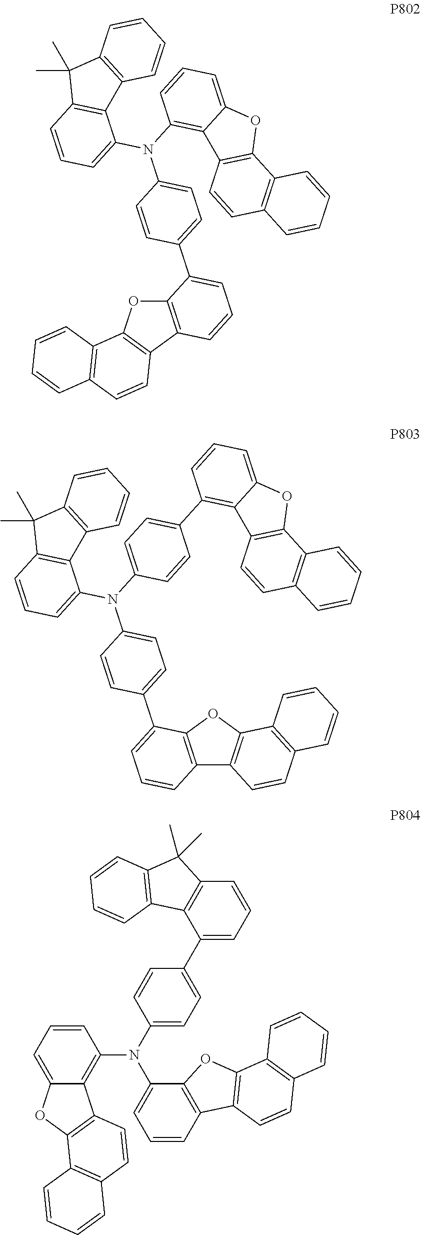

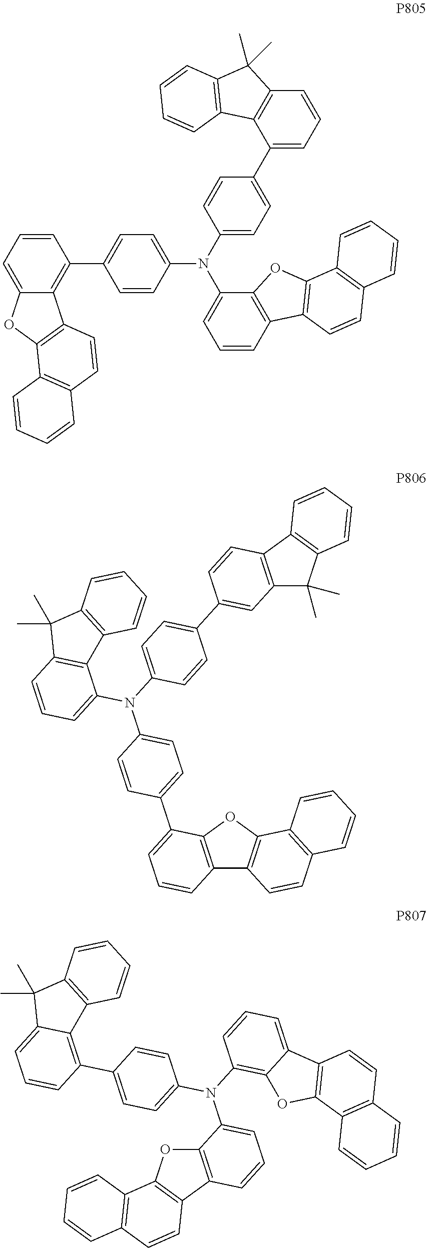 [SMILES: CC1(C)c2ccccc2-c2c(-c3ccc(N(c4ccc(-c5cccc6oc7c8ccccc8ccc7c56)cc4)c4cccc5c4oc4c6ccccc6ccc54)cc3)cccc21 ; CC1(C)c2ccccc2-c2ccc(-c3ccc(N(c4ccc(-c5cccc6c5oc5c7ccccc7ccc65)cc4)c4cccc5c4-c4ccccc4C5(C)C)cc3)cc21 ; CC1(C)c2ccccc2-c2c(-c3ccc(N(c4cccc5c4oc4c6ccccc6ccc54)c4cccc5c4oc4c6ccccc6ccc54)cc3)cccc21]

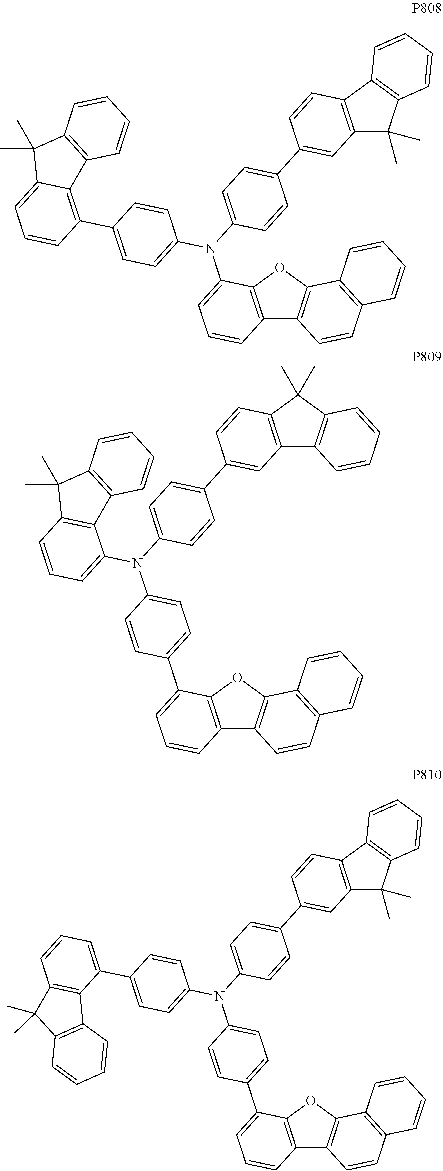

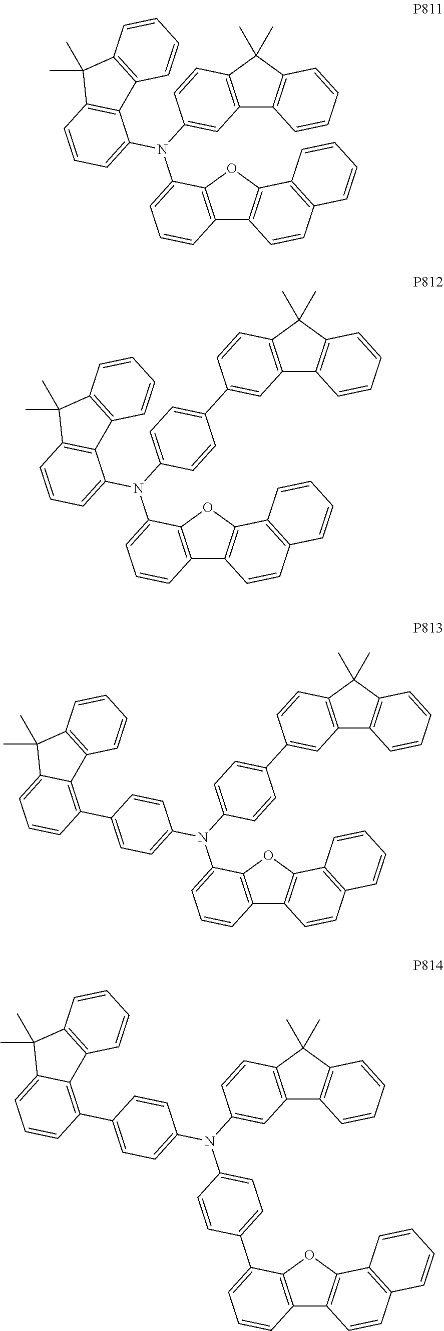 [SMILES: CC1(C)c2ccccc2-c2cc(N(c3cccc4c3-c3ccccc3C4(C)C)c3cccc4c3oc3c5ccccc5ccc43)ccc21 ; CC1(C)c2ccccc2-c2cc(-c3ccc(N(c4cccc5c4-c4ccccc4C5(C)C)c4cccc5c4oc4c6ccccc6ccc54)cc3)ccc21 ; CC1(C)c2ccccc2-c2cc(-c3ccc(N(c4ccc(-c5cccc6c5-c5ccccc5C6(C)C)cc4)c4cccc5c4oc4c6ccccc6ccc54)cc3)ccc21 ; CC1(C)c2ccccc2-c2cc(N(c3ccc(-c4cccc5c4-c4ccccc4C5(C)C)cc3)c3ccc(-c4cccc5c4oc4c6ccccc6ccc54)cc3)ccc21]

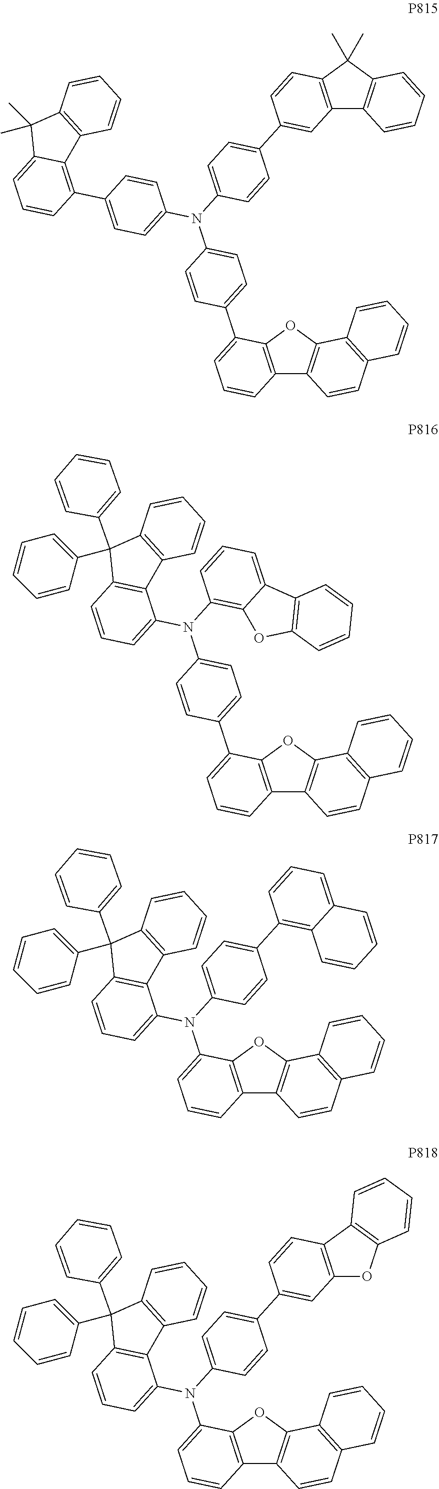 [SMILES: CC1(C)c2ccccc2-c2cc(-c3ccc(N(c4ccc(-c5cccc6c5-c5ccccc5C6(C)C)cc4)c4ccc(-c5cccc6c5oc5c7ccccc7ccc65)cc4)cc3)ccc21 ; c1ccc(C2(c3ccccc3)c3ccccc3-c3c(N(c4ccc(-c5cccc6c5oc5c7ccccc7ccc65)cc4)c4cccc5c4oc4ccccc45)cccc32)cc1 ; c1ccc(C2(c3ccccc3)c3ccccc3-c3c(N(c4ccc(-c5cccc6ccccc56)cc4)c4cccc5c4oc4c6ccccc6ccc54)cccc32)cc1 ; c1ccc(C2(c3ccccc3)c3ccccc3-c3c(N(c4ccc(-c5ccc6c(c5)oc5ccccc56)cc4)c4cccc5c4oc4c6ccccc6ccc54)cccc32)cc1]

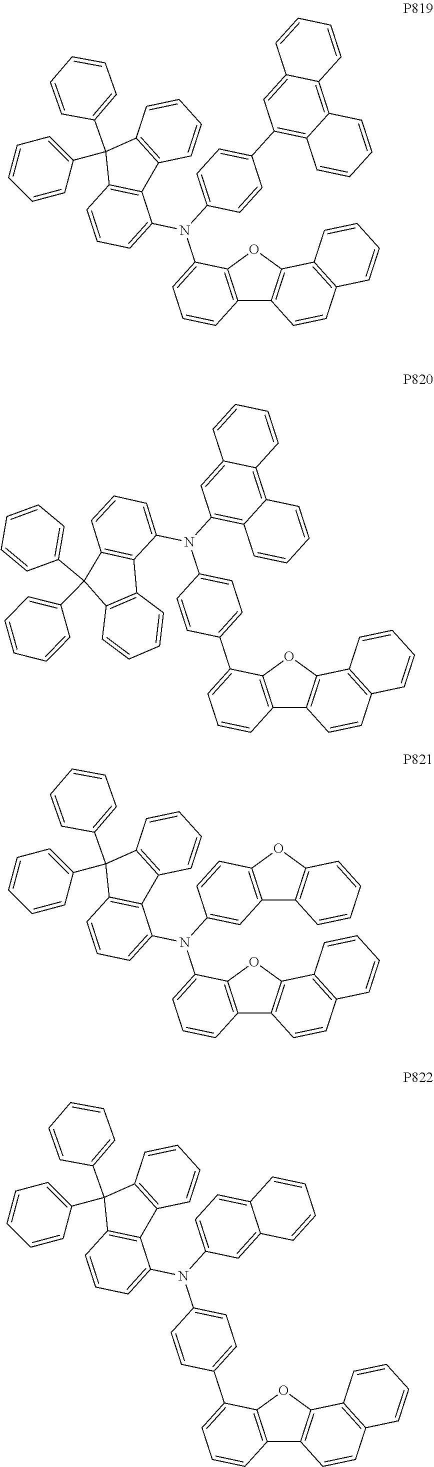 [SMILES: c1ccc(C2(c3ccccc3)c3ccccc3-c3c(N(c4ccc(-c5cc6ccccc6c6ccccc56)cc4)c4cccc5c4oc4c6ccccc6ccc54)cccc32)cc1 ; c1ccc(C2(c3ccccc3)c3ccccc3-c3c(N(c4ccc(-c5cccc6c5oc5c7ccccc7ccc65)cc4)c4cc5ccccc5c5ccccc45)cccc32)cc1 ; c1ccc(C2(c3ccccc3)c3ccccc3-c3c(N(c4ccc5oc6ccccc6c5c4)c4cccc5c4oc4c6ccccc6ccc54)cccc32)cc1 ; c1ccc(C2(c3ccccc3)c3ccccc3-c3c(N(c4ccc(-c5cccc6c5oc5c7ccccc7ccc65)cc4)c4ccc5ccccc5c4)cccc32)cc1]

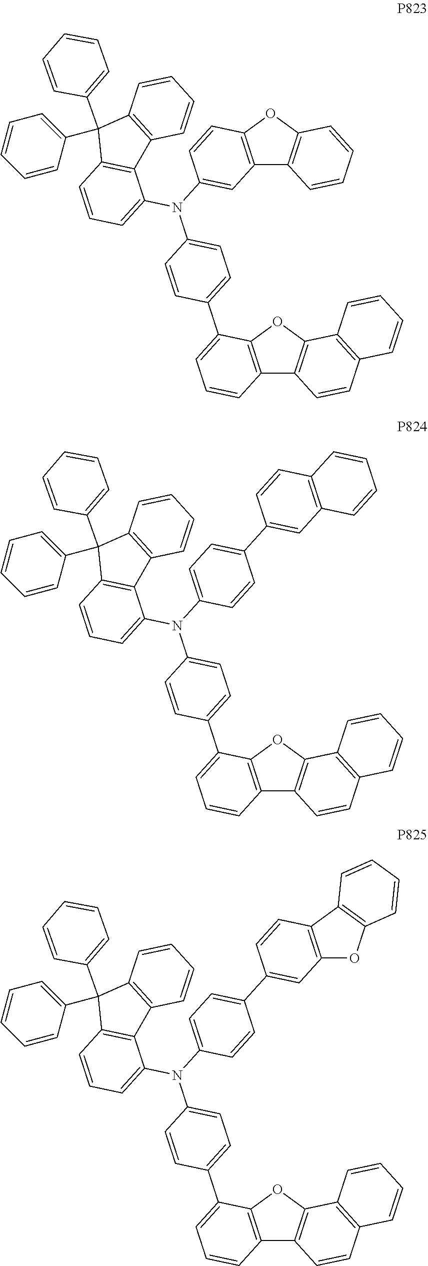 [SMILES: c1ccc(C2(c3ccccc3)c3ccccc3-c3c(N(c4ccc(-c5cccc6c5oc5c7ccccc7ccc65)cc4)c4ccc5oc6ccccc6c5c4)cccc32)cc1 ; c1ccc(C2(c3ccccc3)c3ccccc3-c3c(N(c4ccc(-c5ccc6ccccc6c5)cc4)c4ccc(-c5cccc6c5oc5c7ccccc7ccc65)cc4)cccc32)cc1 ; c1ccc(C2(c3ccccc3)c3ccccc3-c3c(N(c4ccc(-c5ccc6c(c5)oc5ccccc56)cc4)c4ccc(-c5cccc6c5oc5c7ccccc7ccc65)cc4)cccc32)cc1]

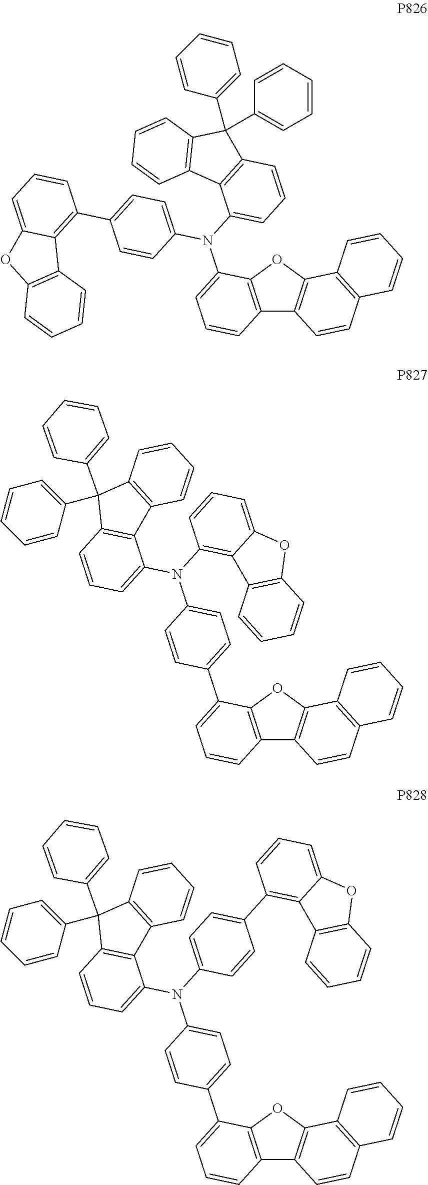 [SMILES: c1ccc(C2(c3ccccc3)c3ccccc3-c3c(N(c4ccc(-c5cccc6oc7ccccc7c56)cc4)c4cccc5c4oc4c6ccccc6ccc54)cccc32)cc1 ; c1ccc(C2(c3ccccc3)c3ccccc3-c3c(N(c4ccc(-c5cccc6c5oc5c7ccccc7ccc65)cc4)c4cccc5oc6ccccc6c45)cccc32)cc1 ; c1ccc(C2(c3ccccc3)c3ccccc3-c3c(N(c4ccc(-c5cccc6c5oc5c7ccccc7ccc65)cc4)c4ccc(-c5cccc6oc7ccccc7c56)cc4)cccc32)cc1]

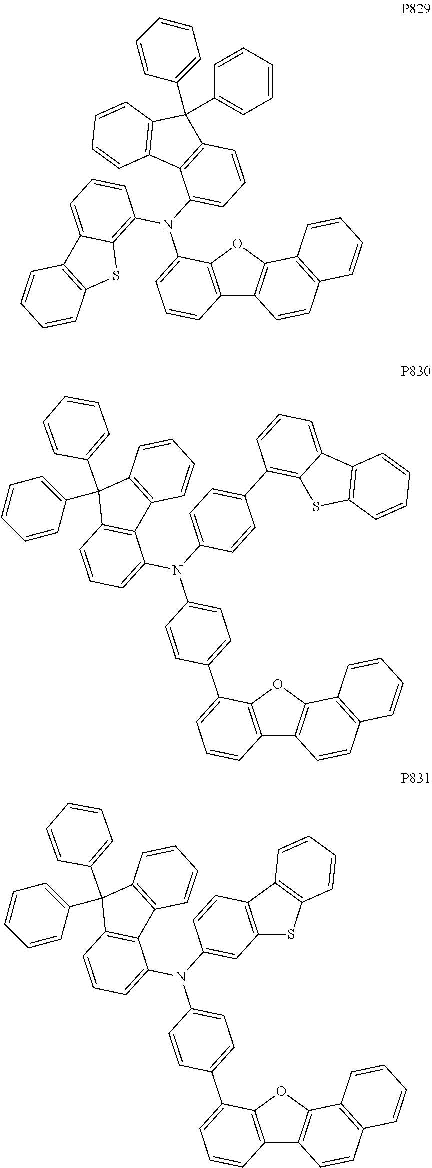 [SMILES: c1ccc(C2(c3ccccc3)c3ccccc3-c3c(N(c4cccc5c4oc4c6ccccc6ccc54)c4cccc5c4sc4ccccc45)cccc32)cc1 ; c1ccc(C2(c3ccccc3)c3ccccc3-c3c(N(c4ccc(-c5cccc6c5oc5c7ccccc7ccc65)cc4)c4ccc(-c5cccc6c5sc5ccccc56)cc4)cccc32)cc1 ; c1ccc(C2(c3ccccc3)c3ccccc3-c3c(N(c4ccc(-c5cccc6c5oc5c7ccccc7ccc65)cc4)c4ccc5c(c4)sc4ccccc45)cccc32)cc1]

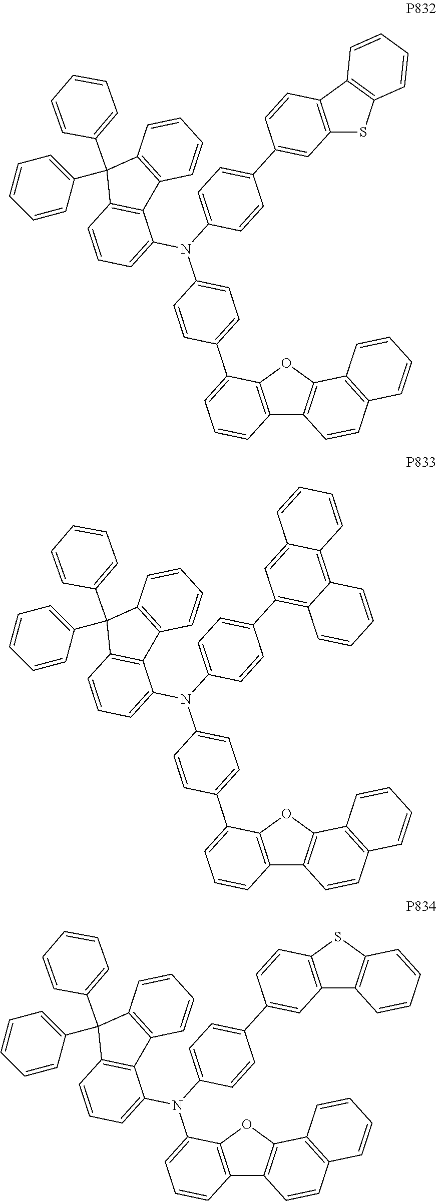 [SMILES: c1ccc(C2(c3ccccc3)c3ccccc3-c3c(N(c4ccc(-c5ccc6c(c5)sc5ccccc56)cc4)c4ccc(-c5cccc6c5oc5c7ccccc7ccc65)cc4)cccc32)cc1 ; c1ccc(C2(c3ccccc3)c3ccccc3-c3c(N(c4ccc(-c5cc6ccccc6c6ccccc56)cc4)c4ccc(-c5cccc6c5oc5c7ccccc7ccc65)cc4)cccc32)cc1 ; c1ccc(C2(c3ccccc3)c3ccccc3-c3c(N(c4ccc(-c5ccc6sc7ccccc7c6c5)cc4)c4cccc5c4oc4c6ccccc6ccc54)cccc32)cc1]

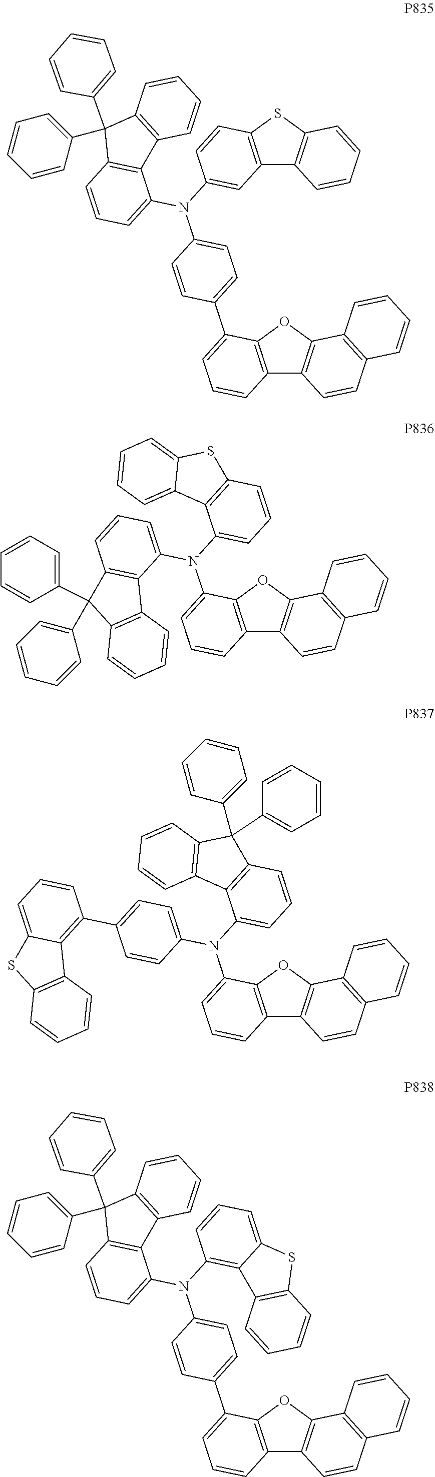 [SMILES: c1ccc(C2(c3ccccc3)c3ccccc3-c3c(N(c4ccc(-c5cccc6c5oc5c7ccccc7ccc65)cc4)c4ccc5sc6ccccc6c5c4)cccc32)cc1 ; c1ccc(C2(c3ccccc3)c3ccccc3-c3c(N(c4cccc5c4oc4c6ccccc6ccc54)c4cccc5sc6ccccc6c45)cccc32)cc1 ; c1ccc(C2(c3ccccc3)c3ccccc3-c3c(N(c4ccc(-c5cccc6sc7ccccc7c56)cc4)c4cccc5c4oc4c6ccccc6ccc54)cccc32)cc1 ; c1ccc(C2(c3ccccc3)c3ccccc3-c3c(N(c4ccc(-c5cccc6c5oc5c7ccccc7ccc65)cc4)c4cccc5sc6ccccc6c45)cccc32)cc1]

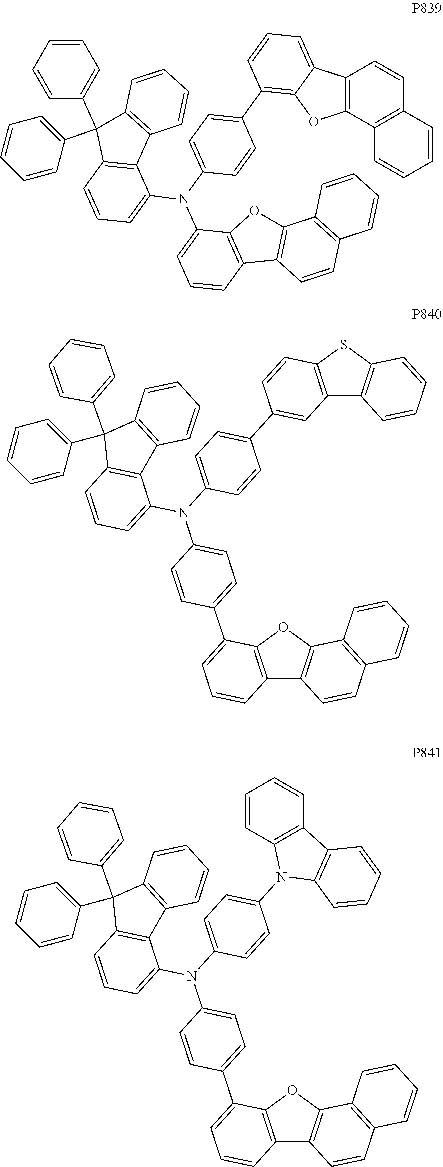 [SMILES: c1ccc(C2(c3ccccc3)c3ccccc3-c3c(N(c4ccc(-c5cccc6c5oc5c7ccccc7ccc65)cc4)c4cccc5c4oc4c6ccccc6ccc54)cccc32)cc1 ; c1ccc(C2(c3ccccc3)c3ccccc3-c3c(N(c4ccc(-c5ccc6sc7ccccc7c6c5)cc4)c4ccc(-c5cccc6c5oc5c7ccccc7ccc65)cc4)cccc32)cc1 ; c1ccc(C2(c3ccccc3)c3ccccc3-c3c(N(c4ccc(-c5cccc6c5oc5c7ccccc7ccc65)cc4)c4ccc(-n5c6ccccc6c6ccccc65)cc4)cccc32)cc1]

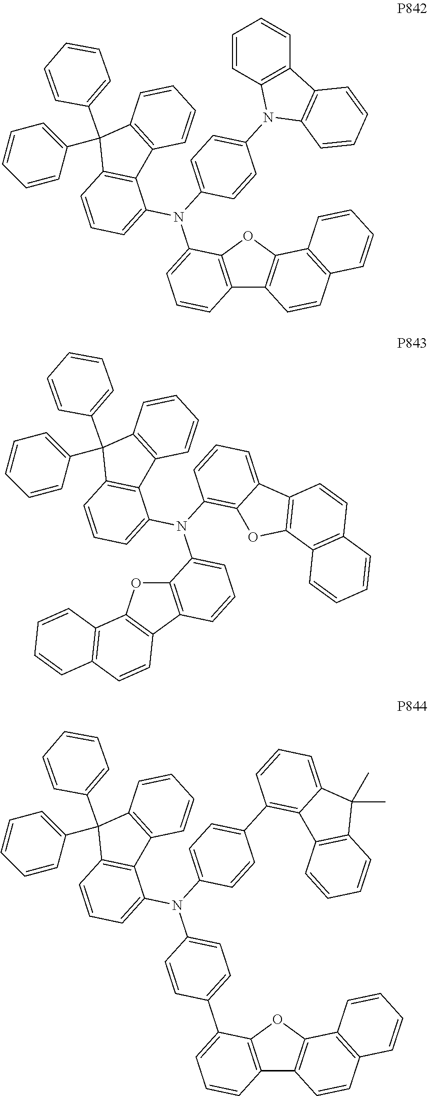 [SMILES: c1ccc(C2(c3ccccc3)c3ccccc3-c3c(N(c4ccc(-n5c6ccccc6c6ccccc65)cc4)c4cccc5c4oc4c6ccccc6ccc54)cccc32)cc1 ; c1ccc(C2(c3ccccc3)c3ccccc3-c3c(N(c4cccc5c4oc4c6ccccc6ccc54)c4cccc5c4oc4c6ccccc6ccc54)cccc32)cc1 ; CC1(C)c2ccccc2-c2c(-c3ccc(N(c4ccc(-c5cccc6c5oc5c7ccccc7ccc65)cc4)c4cccc5c4-c4ccccc4C5(c4ccccc4)c4ccccc4)cc3)cccc21]

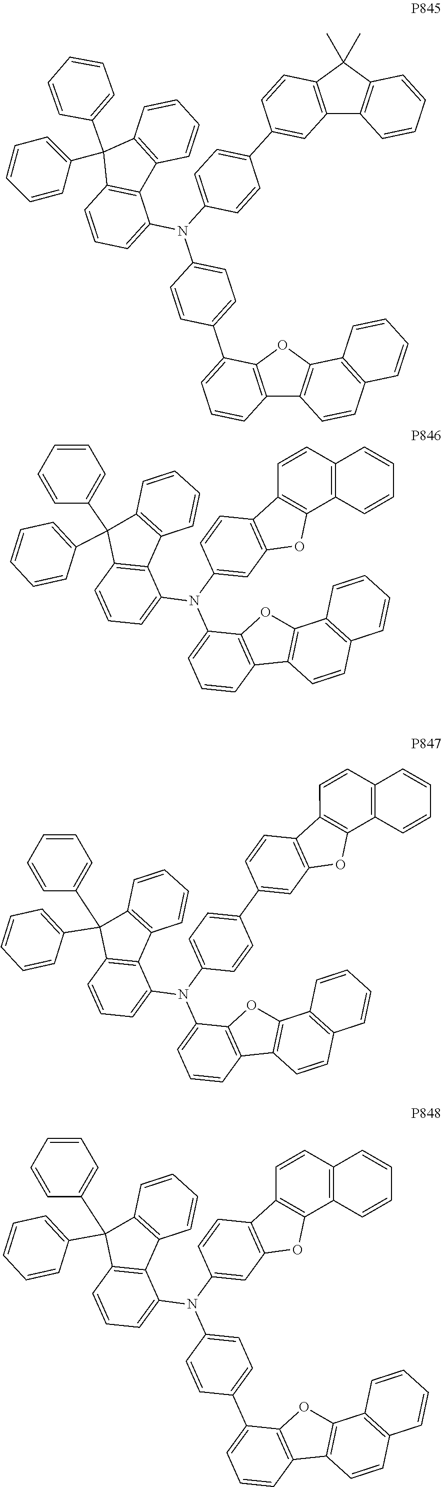

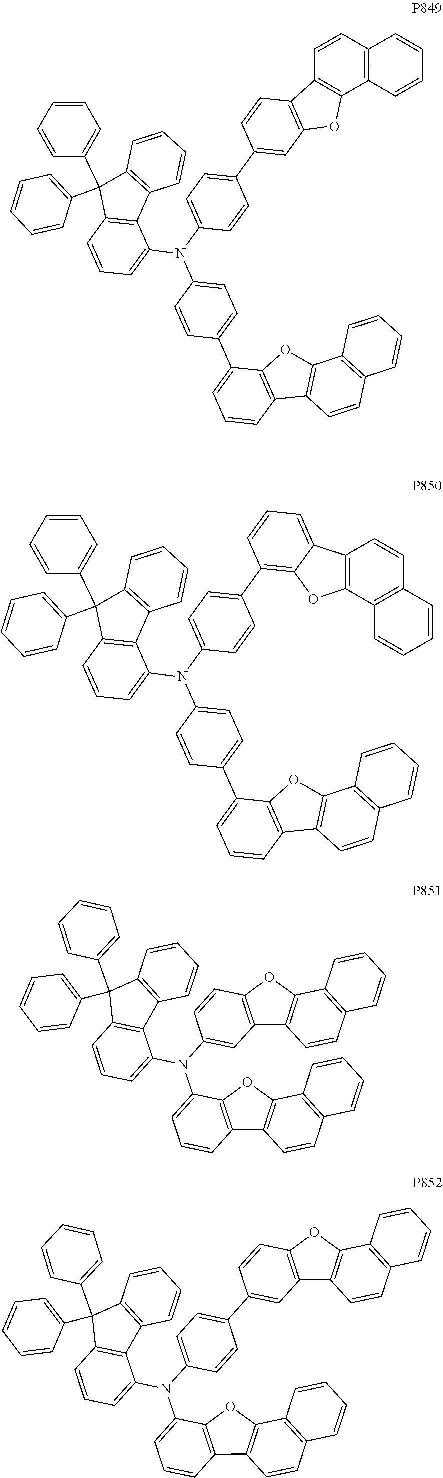 [SMILES: c1ccc(C2(c3ccccc3)c3ccccc3-c3c(N(c4ccc(-c5ccc6c(c5)oc5c7ccccc7ccc65)cc4)c4ccc(-c5cccc6c5oc5c7ccccc7ccc65)cc4)cccc32)cc1 ; c1ccc(C2(c3ccccc3)c3ccccc3-c3c(N(c4ccc(-c5cccc6c5oc5c7ccccc7ccc65)cc4)c4ccc(-c5cccc6c5oc5c7ccccc7ccc65)cc4)cccc32)cc1 ; c1ccc(C2(c3ccccc3)c3ccccc3-c3c(N(c4ccc5oc6c7ccccc7ccc6c5c4)c4cccc5c4oc4c6ccccc6ccc54)cccc32)cc1 ; c1ccc(C2(c3ccccc3)c3ccccc3-c3c(N(c4ccc(-c5ccc6oc7c8ccccc8ccc7c6c5)cc4)c4cccc5c4oc4c6ccccc6ccc54)cccc32)cc1]

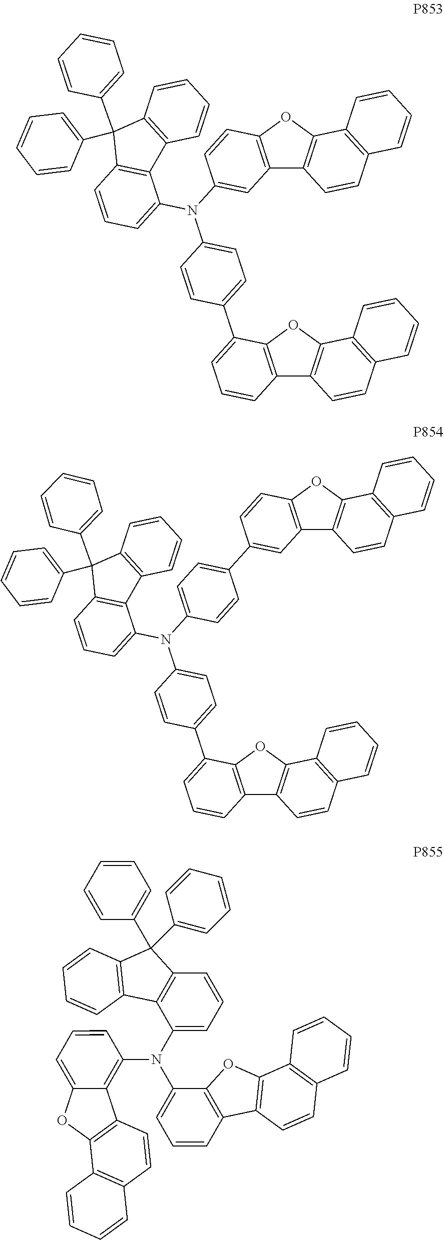 [SMILES: c1ccc(C2(c3ccccc3)c3ccccc3-c3c(N(c4ccc(-c5cccc6c5oc5c7ccccc7ccc65)cc4)c4ccc5oc6c7ccccc7ccc6c5c4)cccc32)cc1 ; c1ccc(C2(c3ccccc3)c3ccccc3-c3c(N(c4ccc(-c5ccc6oc7c8ccccc8ccc7c6c5)cc4)c4ccc(-c5cccc6c5oc5c7ccccc7ccc65)cc4)cccc32)cc1 ; c1ccc(C2(c3ccccc3)c3ccccc3-c3c(N(c4cccc5c4oc4c6ccccc6ccc54)c4cccc5oc6c7ccccc7ccc6c45)cccc32)cc1]

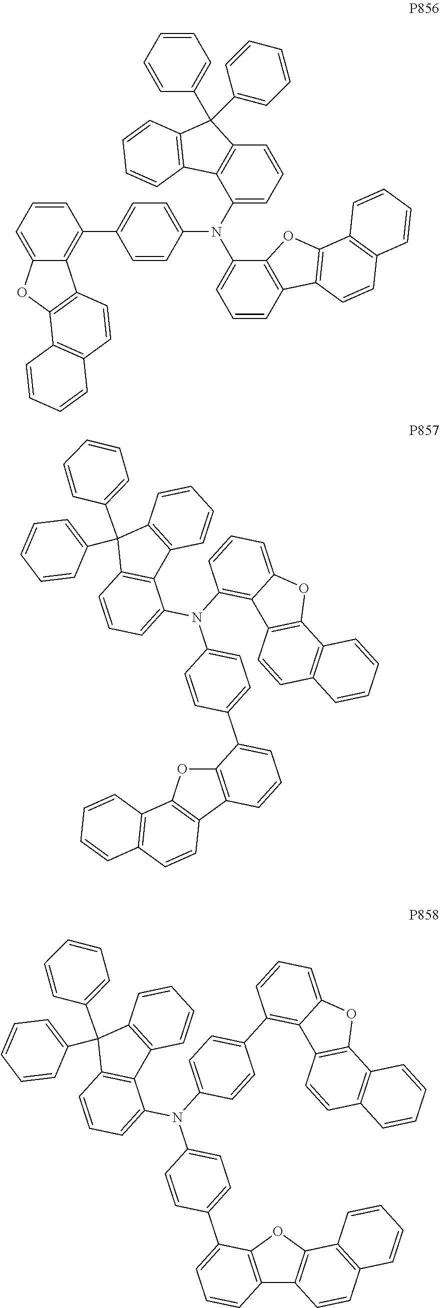

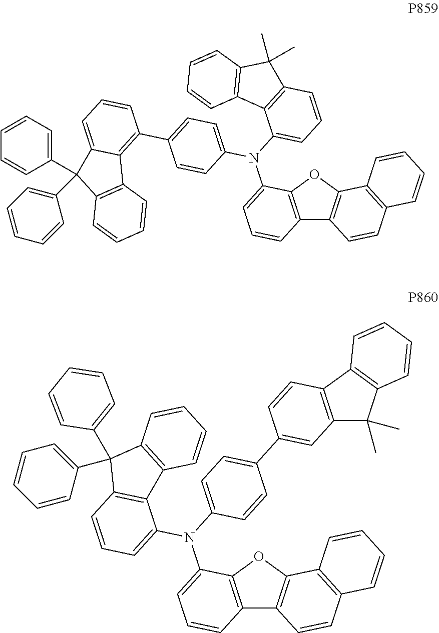 [SMILES: CC1(C)c2ccccc2-c2c(N(c3ccc(-c4cccc5c4-c4ccccc4C5(c4ccccc4)c4ccccc4)cc3)c3cccc4c3oc3c5ccccc5ccc43)cccc21 ; CC1(C)c2ccccc2-c2ccc(-c3ccc(N(c4cccc5c4-c4ccccc4C5(c4ccccc4)c4ccccc4)c4cccc5c4oc4c6ccccc6ccc54)cc3)cc21]

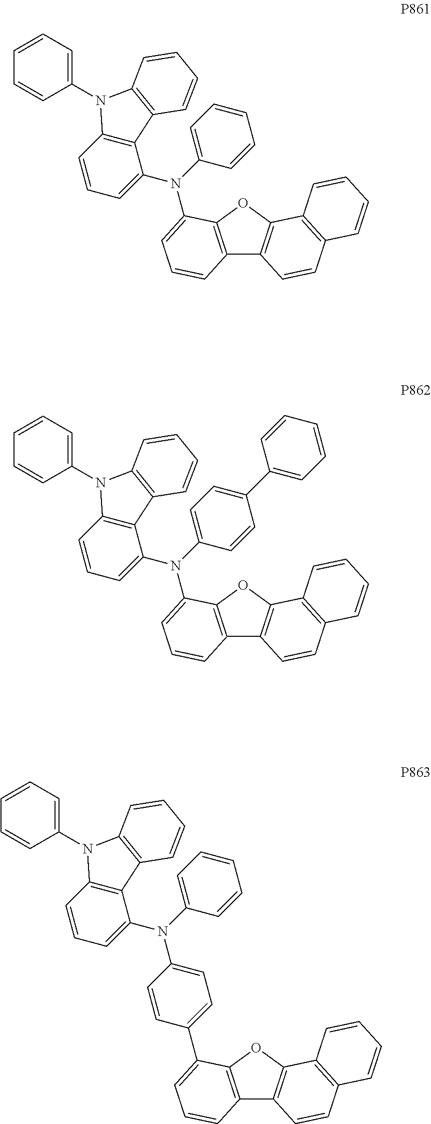 [SMILES: c1ccc(N(c2cccc3c2oc2c4ccccc4ccc32)c2cccc3c2c2ccccc2n3-c2ccccc2)cc1 ; c1ccc(-c2ccc(N(c3cccc4c3oc3c5ccccc5ccc43)c3cccc4c3c3ccccc3n4-c3ccccc3)cc2)cc1 ; c1ccc(N(c2ccc(-c3cccc4c3oc3c5ccccc5ccc43)cc2)c2cccc3c2c2ccccc2n3-c2ccccc2)cc1]

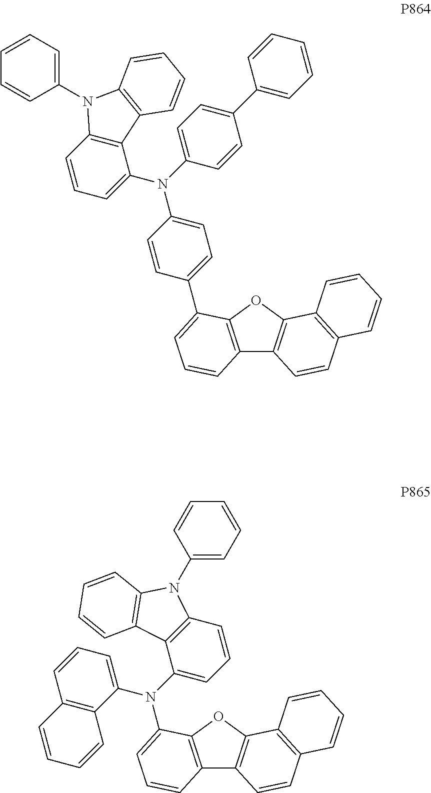 [SMILES: c1ccc(-c2ccc(N(c3ccc(-c4cccc5c4oc4c6ccccc6ccc54)cc3)c3cccc4c3c3ccccc3n4-c3ccccc3)cc2)cc1 ; c1ccc(-n2c3ccccc3c3c(N(c4cccc5ccccc45)c4cccc5c4oc4c6ccccc6ccc54)cccc32)cc1]

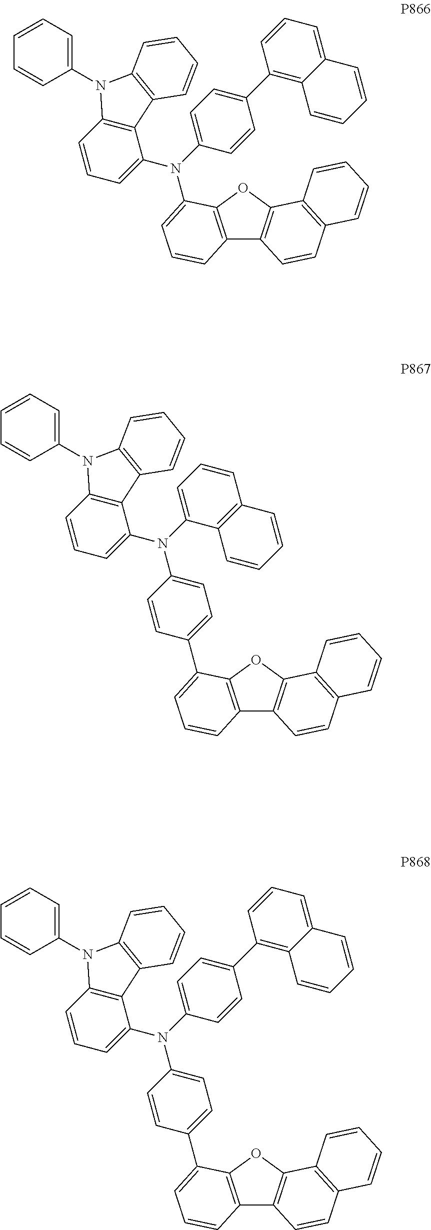 [SMILES: c1ccc(-n2c3ccccc3c3c(N(c4ccc(-c5cccc6ccccc56)cc4)c4cccc5c4oc4c6ccccc6ccc54)cccc32)cc1 ; c1ccc(-n2c3ccccc3c3c(N(c4ccc(-c5cccc6c5oc5c7ccccc7ccc65)cc4)c4cccc5ccccc45)cccc32)cc1 ; c1ccc(-n2c3ccccc3c3c(N(c4ccc(-c5cccc6ccccc56)cc4)c4ccc(-c5cccc6c5oc5c7ccccc7ccc65)cc4)cccc32)cc1]

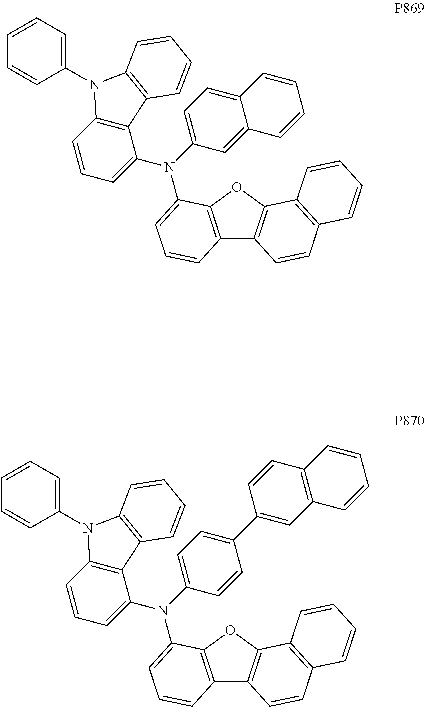 [SMILES: c1ccc(-n2c3ccccc3c3c(N(c4ccc5ccccc5c4)c4cccc5c4oc4c6ccccc6ccc54)cccc32)cc1 ; c1ccc(-n2c3ccccc3c3c(N(c4ccc(-c5ccc6ccccc6c5)cc4)c4cccc5c4oc4c6ccccc6ccc54)cccc32)cc1]

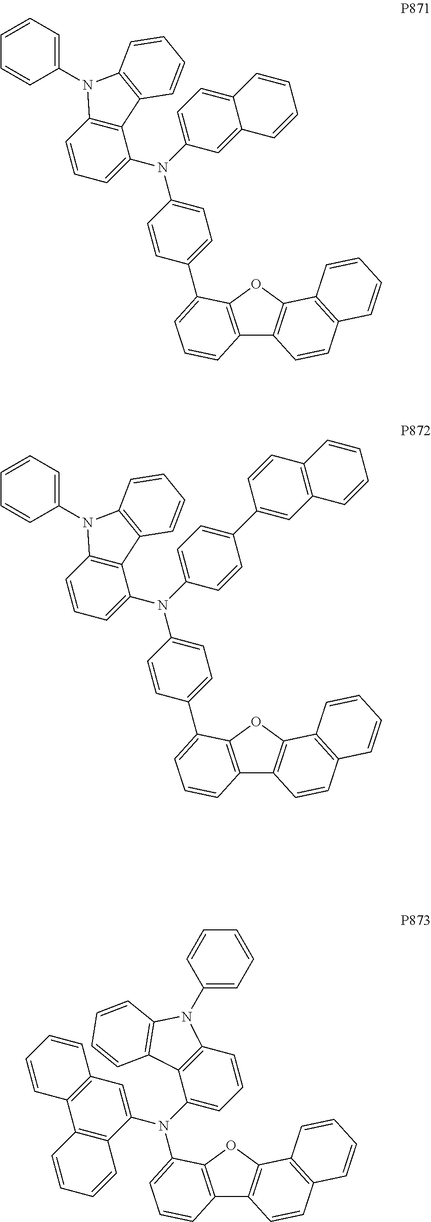 [SMILES: c1ccc(-n2c3ccccc3c3c(N(c4ccc(-c5cccc6c5oc5c7ccccc7ccc65)cc4)c4ccc5ccccc5c4)cccc32)cc1 ; c1ccc(-n2c3ccccc3c3c(N(c4ccc(-c5ccc6ccccc6c5)cc4)c4ccc(-c5cccc6c5oc5c7ccccc7ccc65)cc4)cccc32)cc1 ; c1ccc(-n2c3ccccc3c3c(N(c4cc5ccccc5c5ccccc45)c4cccc5c4oc4c6ccccc6ccc54)cccc32)cc1]

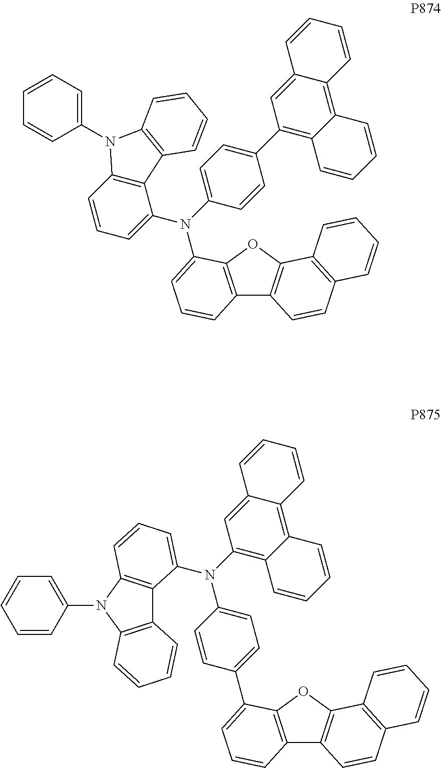 [SMILES: c1ccc(-n2c3ccccc3c3c(N(c4ccc(-c5cc6ccccc6c6ccccc56)cc4)c4cccc5c4oc4c6ccccc6ccc54)cccc32)cc1 ; c1ccc(-n2c3ccccc3c3c(N(c4ccc(-c5cccc6c5oc5c7ccccc7ccc65)cc4)c4cc5ccccc5c5ccccc45)cccc32)cc1]

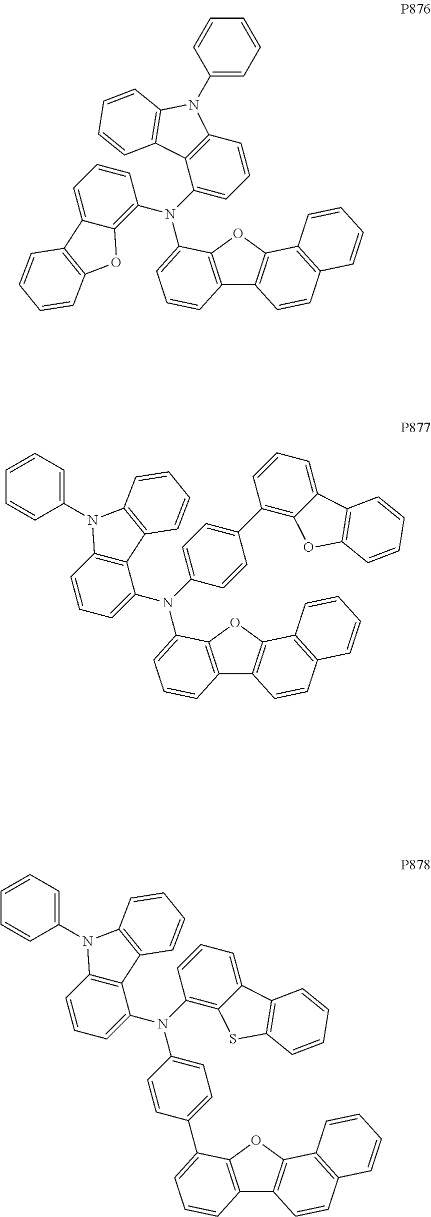 [SMILES: c1ccc(-n2c3ccccc3c3c(N(c4cccc5c4oc4ccccc45)c4cccc5c4oc4c6ccccc6ccc54)cccc32)cc1 ; c1ccc(-n2c3ccccc3c3c(N(c4ccc(-c5cccc6c5oc5ccccc56)cc4)c4cccc5c4oc4c6ccccc6ccc54)cccc32)cc1 ; c1ccc(-n2c3ccccc3c3c(N(c4ccc(-c5cccc6c5oc5c7ccccc7ccc65)cc4)c4cccc5c4sc4ccccc45)cccc32)cc1]

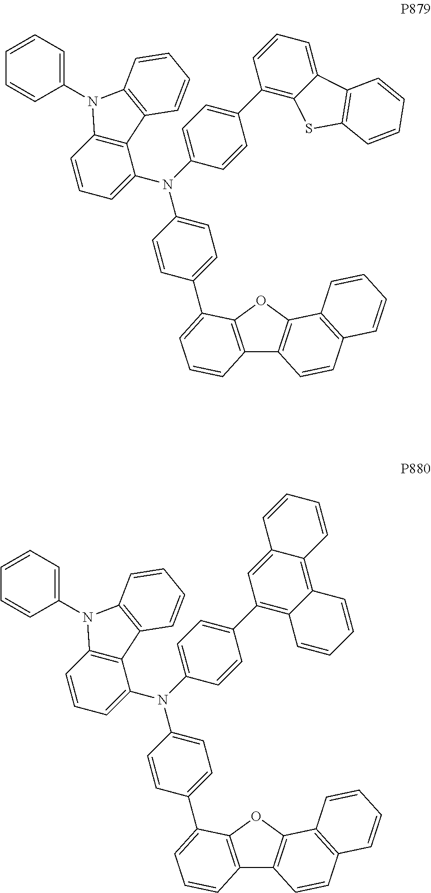 [SMILES: c1ccc(-n2c3ccccc3c3c(N(c4ccc(-c5cccc6c5oc5c7ccccc7ccc65)cc4)c4ccc(-c5cccc6c5sc5ccccc56)cc4)cccc32)cc1 ; c1ccc(-n2c3ccccc3c3c(N(c4ccc(-c5cc6ccccc6c6ccccc56)cc4)c4ccc(-c5cccc6c5oc5c7ccccc7ccc65)cc4)cccc32)cc1]

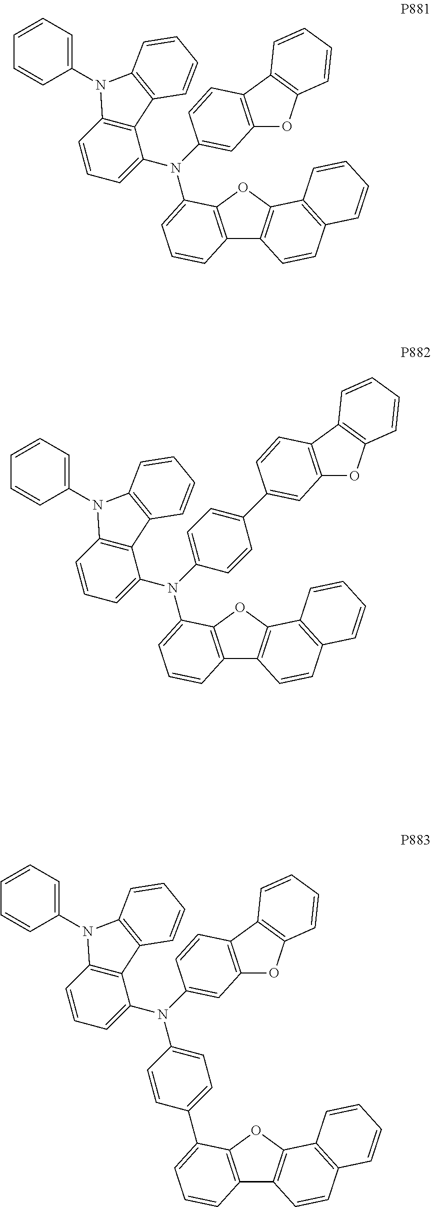 [SMILES: c1ccc(-n2c3ccccc3c3c(N(c4ccc5c(c4)oc4ccccc45)c4cccc5c4oc4c6ccccc6ccc54)cccc32)cc1 ; c1ccc(-n2c3ccccc3c3c(N(c4ccc(-c5ccc6c(c5)oc5ccccc56)cc4)c4cccc5c4oc4c6ccccc6ccc54)cccc32)cc1 ; c1ccc(-n2c3ccccc3c3c(N(c4ccc(-c5cccc6c5oc5c7ccccc7ccc65)cc4)c4ccc5c(c4)oc4ccccc45)cccc32)cc1]

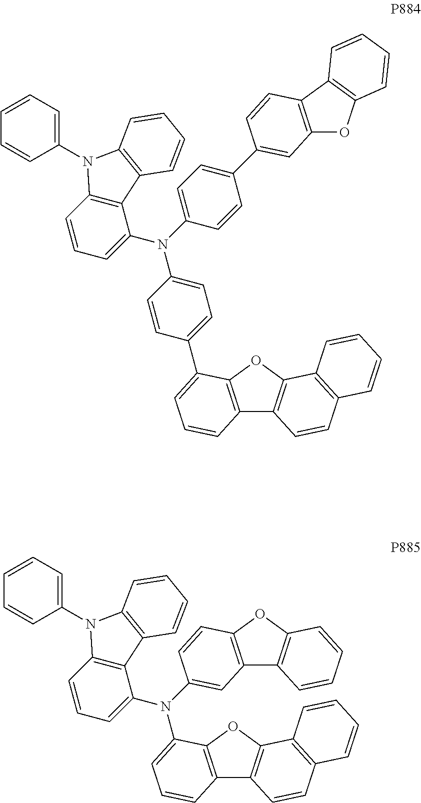 [SMILES: c1ccc(-n2c3ccccc3c3c(N(c4ccc(-c5ccc6c(c5)oc5ccccc56)cc4)c4ccc(-c5cccc6c5oc5c7ccccc7ccc65)cc4)cccc32)cc1 ; c1ccc(-n2c3ccccc3c3c(N(c4ccc5oc6ccccc6c5c4)c4cccc5c4oc4c6ccccc6ccc54)cccc32)cc1]

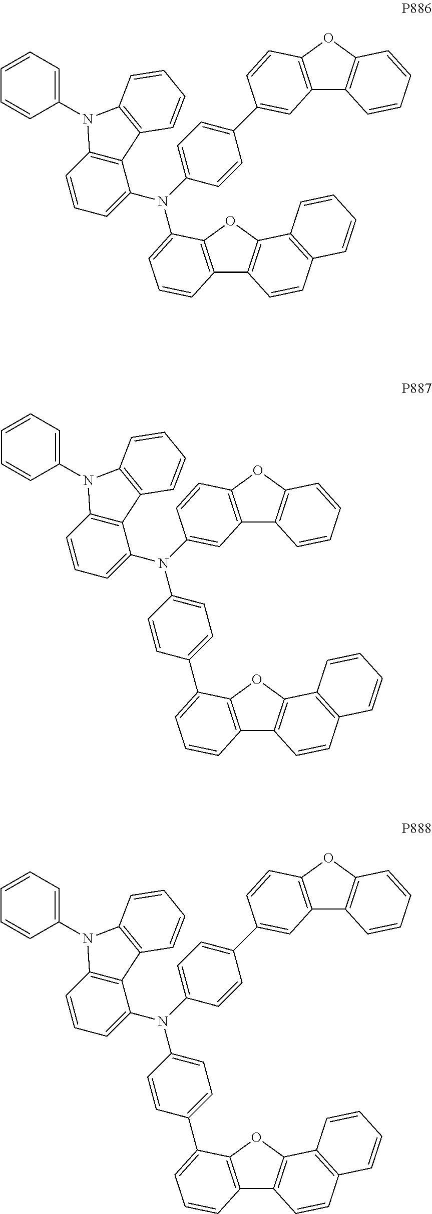 [SMILES: c1ccc(-n2c3ccccc3c3c(N(c4ccc(-c5ccc6oc7ccccc7c6c5)cc4)c4cccc5c4oc4c6ccccc6ccc54)cccc32)cc1 ; c1ccc(-n2c3ccccc3c3c(N(c4ccc(-c5cccc6c5oc5c7ccccc7ccc65)cc4)c4ccc5oc6ccccc6c5c4)cccc32)cc1 ; c1ccc(-n2c3ccccc3c3c(N(c4ccc(-c5ccc6oc7ccccc7c6c5)cc4)c4ccc(-c5cccc6c5oc5c7ccccc7ccc65)cc4)cccc32)cc1]

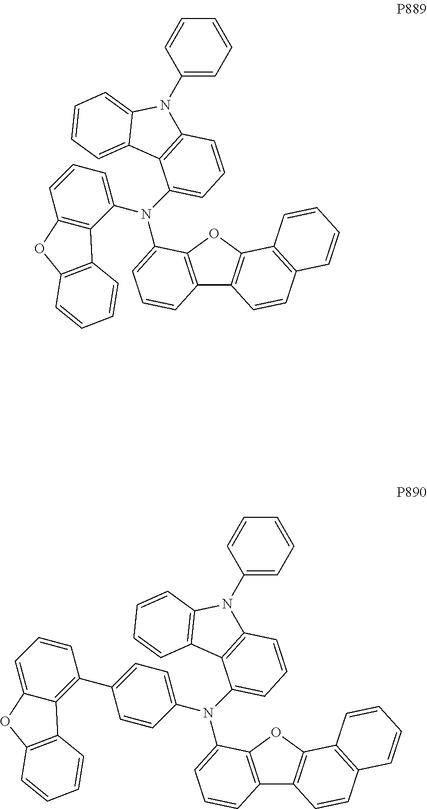 [SMILES: c1ccc(-n2c3ccccc3c3c(N(c4cccc5c4oc4c6ccccc6ccc54)c4cccc5oc6ccccc6c45)cccc32)cc1 ; c1ccc(-n2c3ccccc3c3c(N(c4ccc(-c5cccc6oc7ccccc7c56)cc4)c4cccc5c4oc4c6ccccc6ccc54)cccc32)cc1]

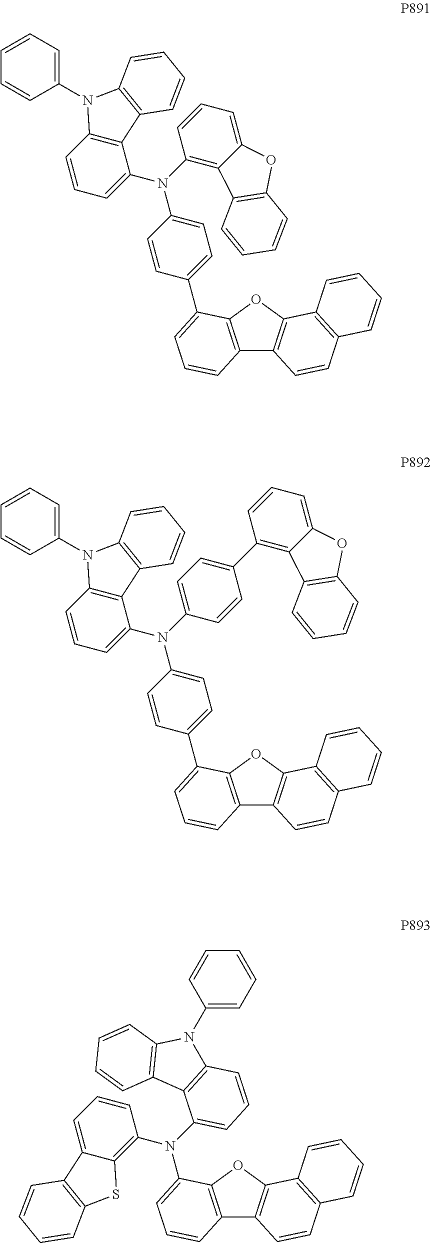 [SMILES: c1ccc(-n2c3ccccc3c3c(N(c4ccc(-c5cccc6c5oc5c7ccccc7ccc65)cc4)c4cccc5oc6ccccc6c45)cccc32)cc1 ; c1ccc(-n2c3ccccc3c3c(N(c4ccc(-c5cccc6c5oc5c7ccccc7ccc65)cc4)c4ccc(-c5cccc6oc7ccccc7c56)cc4)cccc32)cc1 ; c1ccc(-n2c3ccccc3c3c(N(c4cccc5c4oc4c6ccccc6ccc54)c4cccc5c4sc4ccccc45)cccc32)cc1]

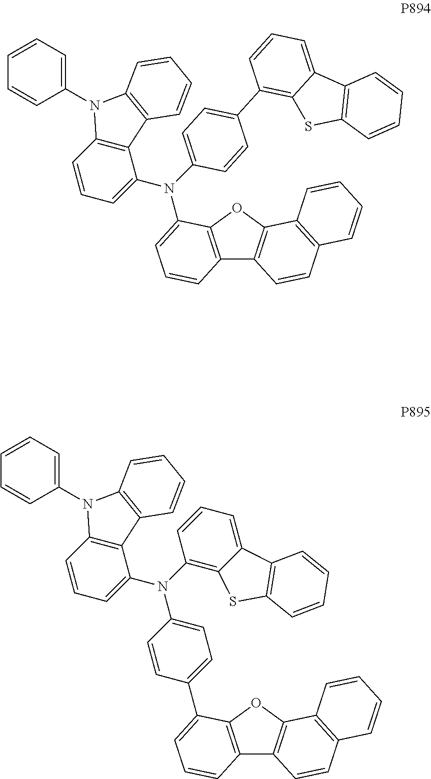 [SMILES: c1ccc(-n2c3ccccc3c3c(N(c4ccc(-c5cccc6c5sc5ccccc56)cc4)c4cccc5c4oc4c6ccccc6ccc54)cccc32)cc1 ; c1ccc(-n2c3ccccc3c3c(N(c4ccc(-c5cccc6c5oc5c7ccccc7ccc65)cc4)c4cccc5c4sc4ccccc45)cccc32)cc1]

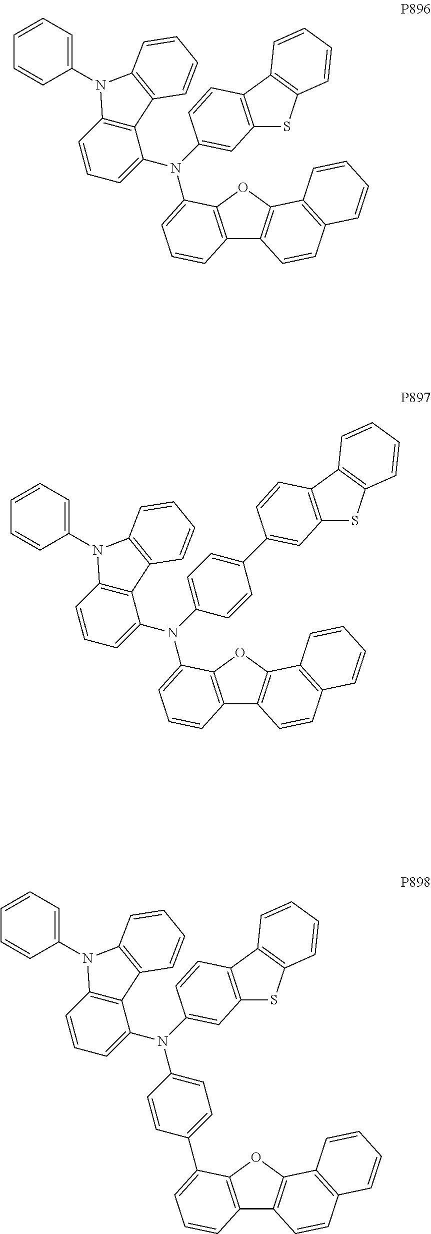 [SMILES: c1ccc(-n2c3ccccc3c3c(N(c4ccc5c(c4)sc4ccccc45)c4cccc5c4oc4c6ccccc6ccc54)cccc32)cc1 ; c1ccc(-n2c3ccccc3c3c(N(c4ccc(-c5ccc6c(c5)sc5ccccc56)cc4)c4cccc5c4oc4c6ccccc6ccc54)cccc32)cc1 ; c1ccc(-n2c3ccccc3c3c(N(c4ccc(-c5cccc6c5oc5c7ccccc7ccc65)cc4)c4ccc5c(c4)sc4ccccc45)cccc32)cc1]

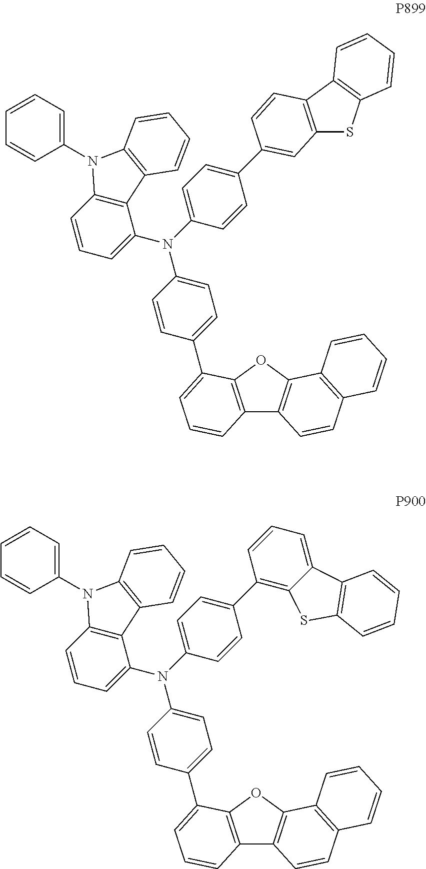 [SMILES: c1ccc(-n2c3ccccc3c3c(N(c4ccc(-c5ccc6c(c5)sc5ccccc56)cc4)c4ccc(-c5cccc6c5oc5c7ccccc7ccc65)cc4)cccc32)cc1 ; c1ccc(-n2c3ccccc3c3c(N(c4ccc(-c5cccc6c5oc5c7ccccc7ccc65)cc4)c4ccc(-c5cccc6c5sc5ccccc56)cc4)cccc32)cc1]

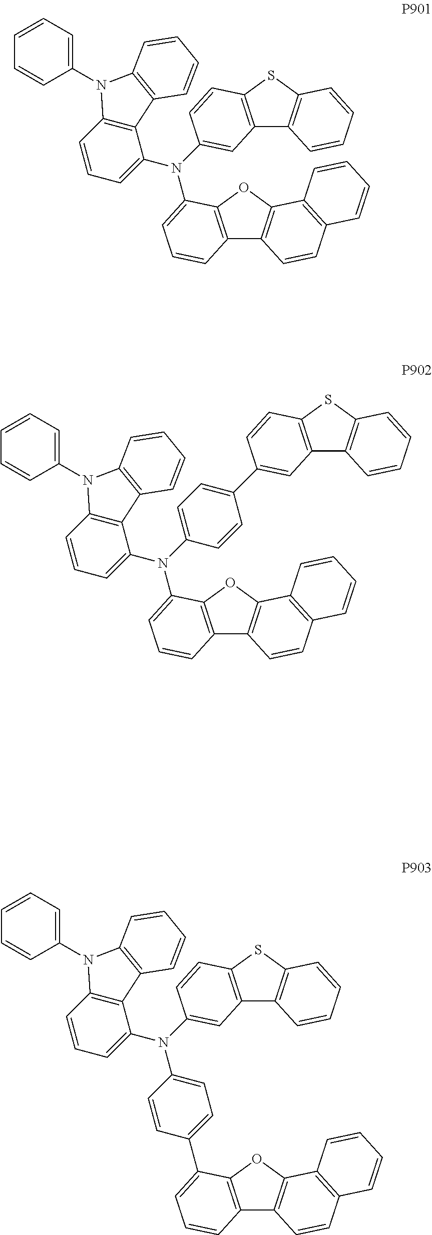 [SMILES: c1ccc(-n2c3ccccc3c3c(N(c4ccc5sc6ccccc6c5c4)c4cccc5c4oc4c6ccccc6ccc54)cccc32)cc1 ; c1ccc(-n2c3ccccc3c3c(N(c4ccc(-c5ccc6sc7ccccc7c6c5)cc4)c4cccc5c4oc4c6ccccc6ccc54)cccc32)cc1 ; c1ccc(-n2c3ccccc3c3c(N(c4ccc(-c5cccc6c5oc5c7ccccc7ccc65)cc4)c4ccc5sc6ccccc6c5c4)cccc32)cc1]

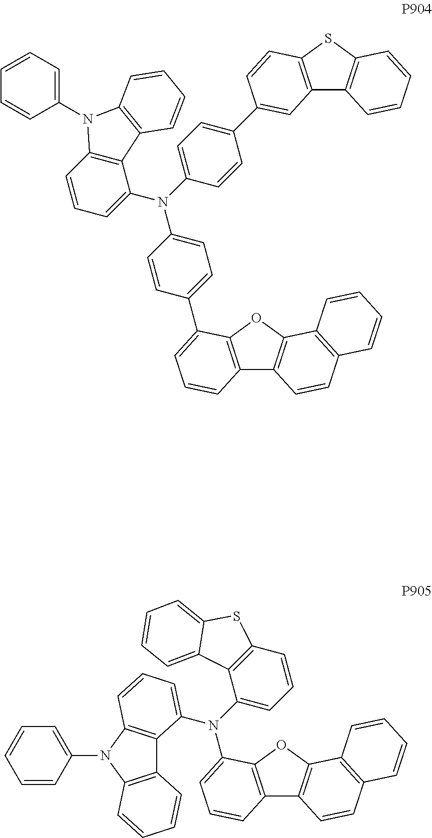 [SMILES: c1ccc(-n2c3ccccc3c3c(N(c4ccc(-c5ccc6sc7ccccc7c6c5)cc4)c4ccc(-c5cccc6c5oc5c7ccccc7ccc65)cc4)cccc32)cc1 ; c1ccc(-n2c3ccccc3c3c(N(c4cccc5c4oc4c6ccccc6ccc54)c4cccc5sc6ccccc6c45)cccc32)cc1]

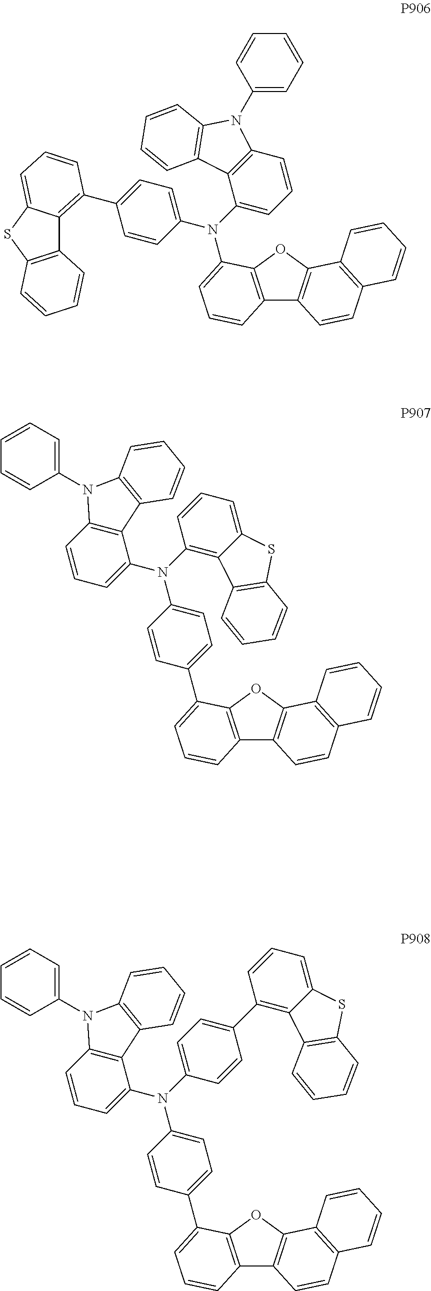 [SMILES: c1ccc(-n2c3ccccc3c3c(N(c4ccc(-c5cccc6sc7ccccc7c56)cc4)c4cccc5c4oc4c6ccccc6ccc54)cccc32)cc1 ; c1ccc(-n2c3ccccc3c3c(N(c4ccc(-c5cccc6c5oc5c7ccccc7ccc65)cc4)c4cccc5sc6ccccc6c45)cccc32)cc1 ; c1ccc(-n2c3ccccc3c3c(N(c4ccc(-c5cccc6c5oc5c7ccccc7ccc65)cc4)c4ccc(-c5cccc6sc7ccccc7c56)cc4)cccc32)cc1]

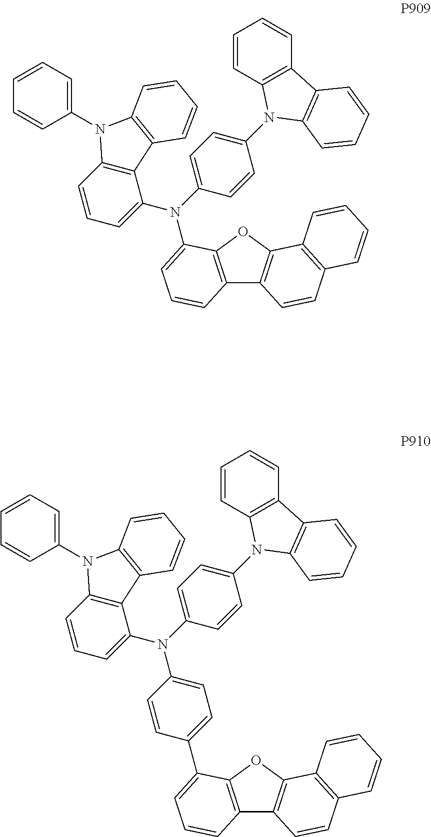 [SMILES: c1ccc(-n2c3ccccc3c3c(N(c4ccc(-n5c6ccccc6c6ccccc65)cc4)c4cccc5c4oc4c6ccccc6ccc54)cccc32)cc1 ; c1ccc(-n2c3ccccc3c3c(N(c4ccc(-c5cccc6c5oc5c7ccccc7ccc65)cc4)c4ccc(-n5c6ccccc6c6ccccc65)cc4)cccc32)cc1]

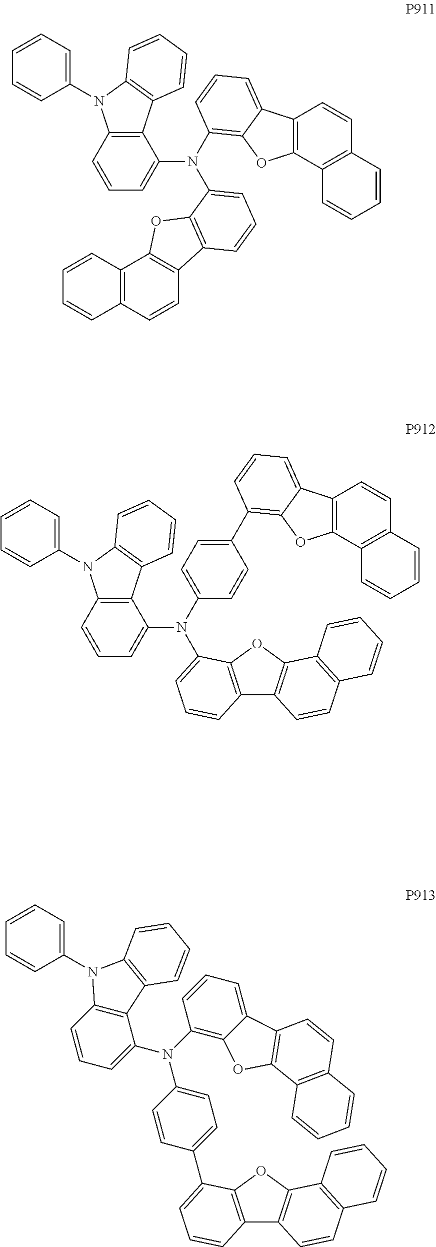 [SMILES: c1ccc(-n2c3ccccc3c3c(N(c4cccc5c4oc4c6ccccc6ccc54)c4cccc5c4oc4c6ccccc6ccc54)cccc32)cc1 ; c1ccc(-n2c3ccccc3c3c(N(c4ccc(-c5cccc6c5oc5c7ccccc7ccc65)cc4)c4cccc5c4oc4c6ccccc6ccc54)cccc32)cc1 ; c1ccc(-n2c3ccccc3c3c(N(c4ccc(-c5cccc6c5oc5c7ccccc7ccc65)cc4)c4cccc5c4oc4c6ccccc6ccc54)cccc32)cc1]

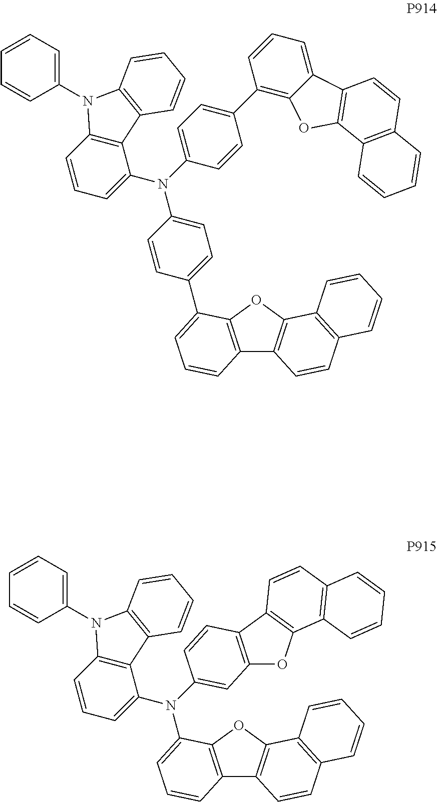 [SMILES: c1ccc(-n2c3ccccc3c3c(N(c4ccc(-c5cccc6c5oc5c7ccccc7ccc65)cc4)c4ccc(-c5cccc6c5oc5c7ccccc7ccc65)cc4)cccc32)cc1 ; c1ccc(-n2c3ccccc3c3c(N(c4ccc5c(c4)oc4c6ccccc6ccc54)c4cccc5c4oc4c6ccccc6ccc54)cccc32)cc1]

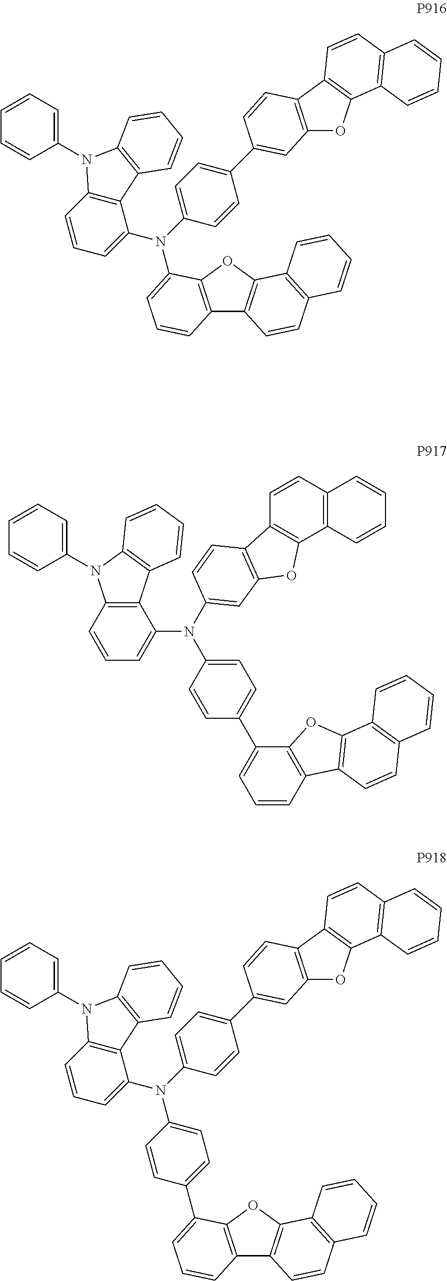 [SMILES: c1ccc(-n2c3ccccc3c3c(N(c4ccc(-c5ccc6c(c5)oc5c7ccccc7ccc65)cc4)c4cccc5c4oc4c6ccccc6ccc54)cccc32)cc1 ; c1ccc(-n2c3ccccc3c3c(N(c4ccc(-c5cccc6c5oc5c7ccccc7ccc65)cc4)c4ccc5c(c4)oc4c6ccccc6ccc54)cccc32)cc1 ; c1ccc(-n2c3ccccc3c3c(N(c4ccc(-c5ccc6c(c5)oc5c7ccccc7ccc65)cc4)c4ccc(-c5cccc6c5oc5c7ccccc7ccc65)cc4)cccc32)cc1]

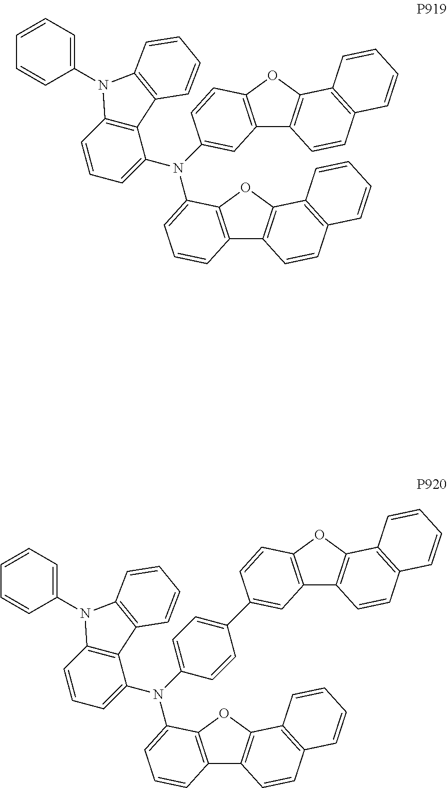 [SMILES: c1ccc(-n2c3ccccc3c3c(N(c4ccc5oc6c7ccccc7ccc6c5c4)c4cccc5c4oc4c6ccccc6ccc54)cccc32)cc1 ; c1ccc(-n2c3ccccc3c3c(N(c4ccc(-c5ccc6oc7c8ccccc8ccc7c6c5)cc4)c4cccc5c4oc4c6ccccc6ccc54)cccc32)cc1]

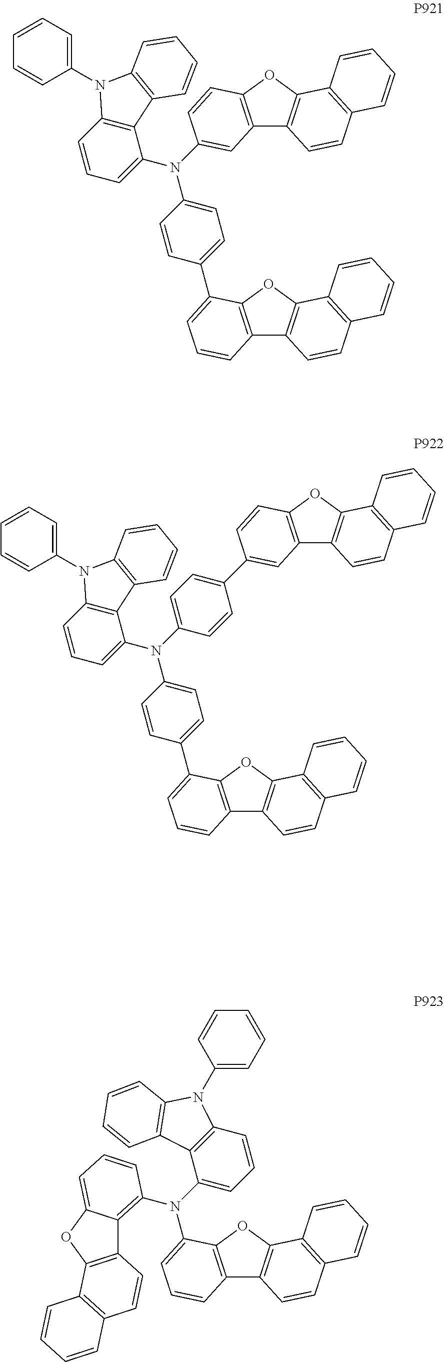 [SMILES: c1ccc(-n2c3ccccc3c3c(N(c4ccc(-c5cccc6c5oc5c7ccccc7ccc65)cc4)c4ccc5oc6c7ccccc7ccc6c5c4)cccc32)cc1 ; c1ccc(-n2c3ccccc3c3c(N(c4ccc(-c5ccc6oc7c8ccccc8ccc7c6c5)cc4)c4ccc(-c5cccc6c5oc5c7ccccc7ccc65)cc4)cccc32)cc1 ; c1ccc(-n2c3ccccc3c3c(N(c4cccc5c4oc4c6ccccc6ccc54)c4cccc5oc6c7ccccc7ccc6c45)cccc32)cc1]

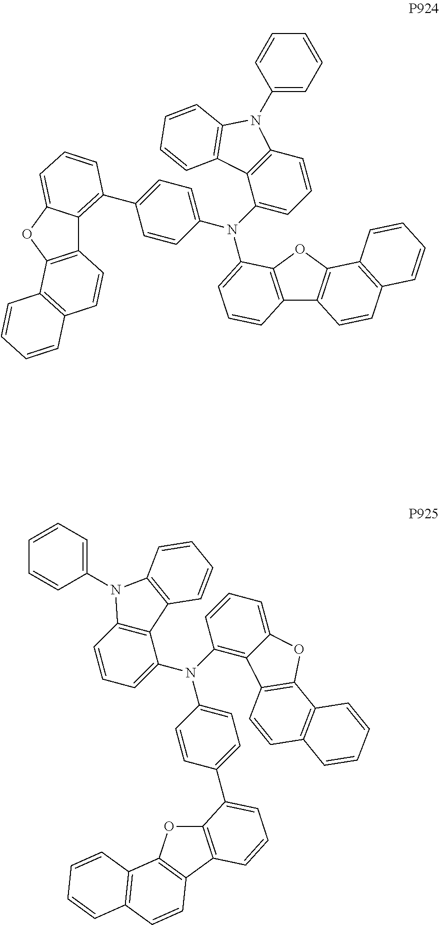 [SMILES: c1ccc(-n2c3ccccc3c3c(N(c4ccc(-c5cccc6oc7c8ccccc8ccc7c56)cc4)c4cccc5c4oc4c6ccccc6ccc54)cccc32)cc1 ; c1ccc(-n2c3ccccc3c3c(N(c4ccc(-c5cccc6c5oc5c7ccccc7ccc65)cc4)c4cccc5oc6c7ccccc7ccc6c45)cccc32)cc1]

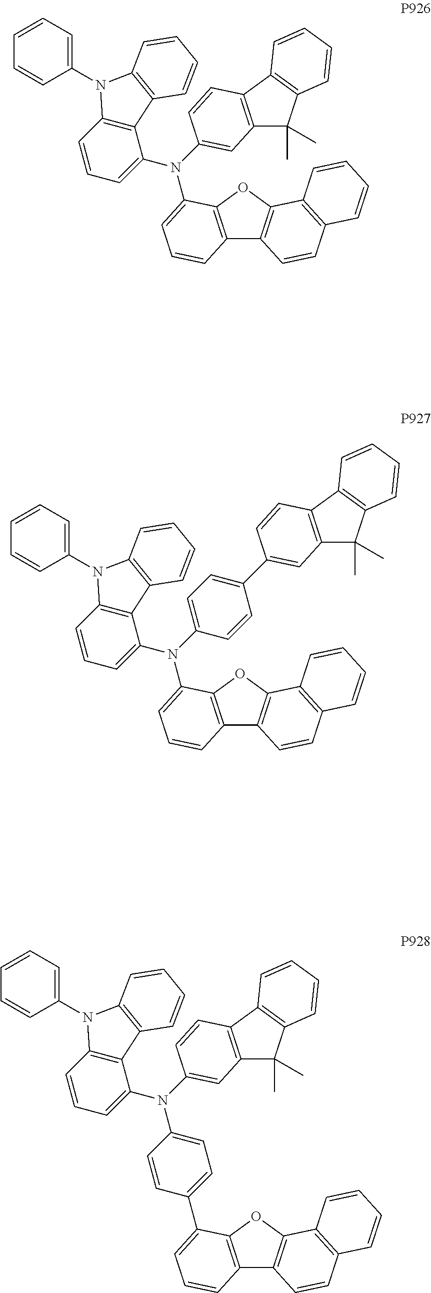 [SMILES: CC1(C)c2ccccc2-c2ccc(N(c3cccc4c3oc3c5ccccc5ccc43)c3cccc4c3c3ccccc3n4-c3ccccc3)cc21 ; CC1(C)c2ccccc2-c2ccc(-c3ccc(N(c4cccc5c4oc4c6ccccc6ccc54)c4cccc5c4c4ccccc4n5-c4ccccc4)cc3)cc21 ; CC1(C)c2ccccc2-c2ccc(N(c3ccc(-c4cccc5c4oc4c6ccccc6ccc54)cc3)c3cccc4c3c3ccccc3n4-c3ccccc3)cc21]

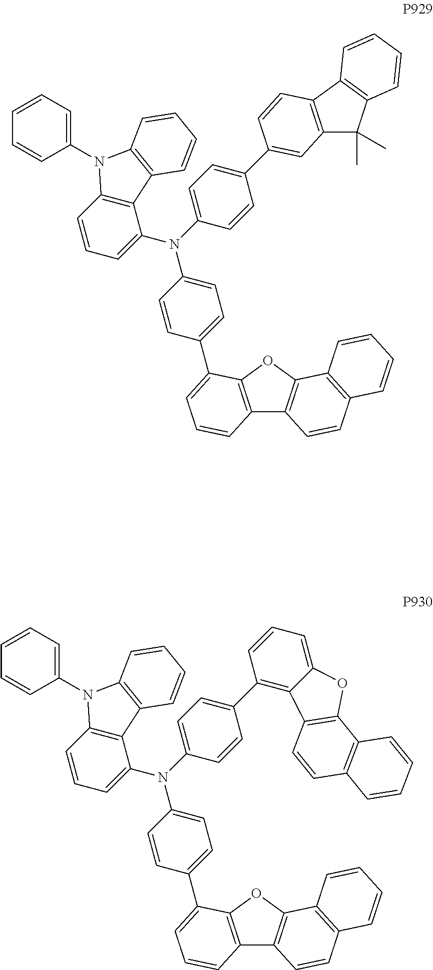 [SMILES: CC1(C)c2ccccc2-c2ccc(-c3ccc(N(c4ccc(-c5cccc6c5oc5c7ccccc7ccc65)cc4)c4cccc5c4c4ccccc4n5-c4ccccc4)cc3)cc21 ; c1ccc(-n2c3ccccc3c3c(N(c4ccc(-c5cccc6c5oc5c7ccccc7ccc65)cc4)c4ccc(-c5cccc6oc7c8ccccc8ccc7c56)cc4)cccc32)cc1]

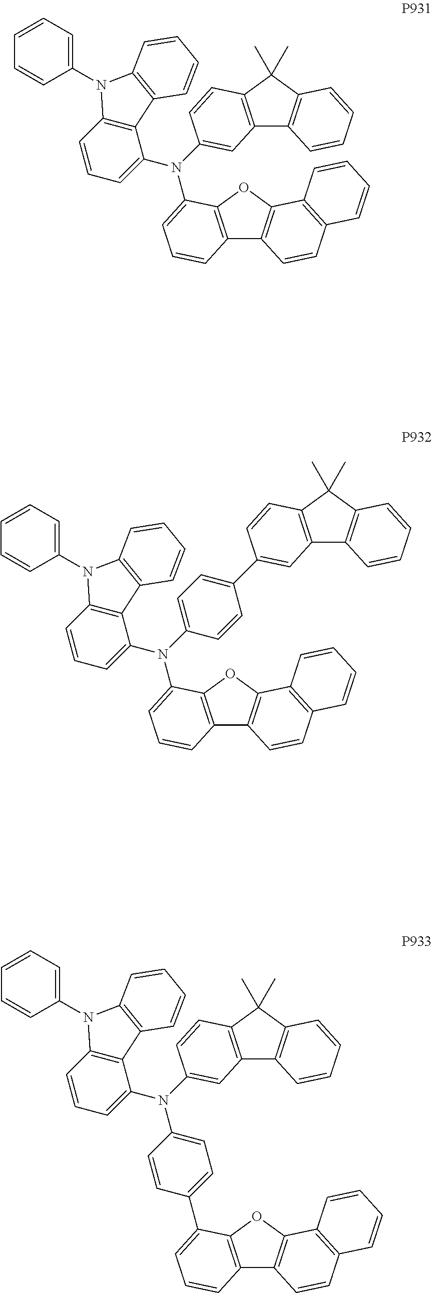 [SMILES: CC1(C)c2ccccc2-c2cc(N(c3cccc4c3oc3c5ccccc5ccc43)c3cccc4c3c3ccccc3n4-c3ccccc3)ccc21 ; CC1(C)c2ccccc2-c2cc(-c3ccc(N(c4cccc5c4oc4c6ccccc6ccc54)c4cccc5c4c4ccccc4n5-c4ccccc4)cc3)ccc21 ; CC1(C)c2ccccc2-c2cc(N(c3ccc(-c4cccc5c4oc4c6ccccc6ccc54)cc3)c3cccc4c3c3ccccc3n4-c3ccccc3)ccc21]

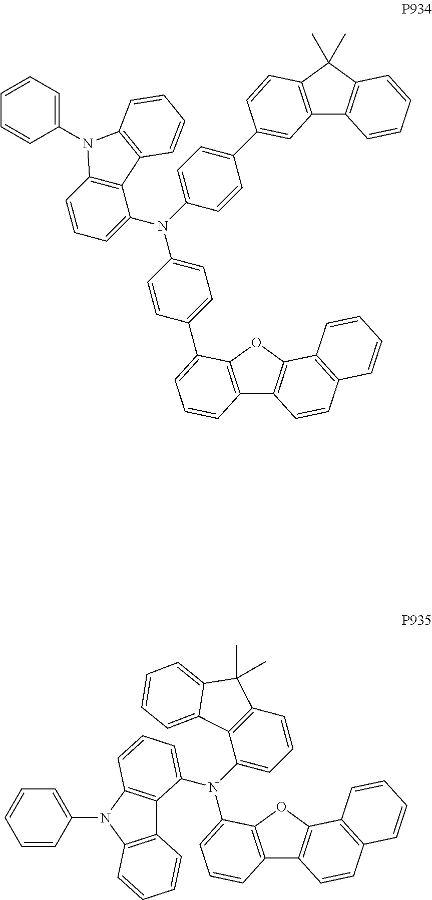 [SMILES: CC1(C)c2ccccc2-c2cc(-c3ccc(N(c4ccc(-c5cccc6c5oc5c7ccccc7ccc65)cc4)c4cccc5c4c4ccccc4n5-c4ccccc4)cc3)ccc21 ; CC1(C)c2ccccc2-c2c(N(c3cccc4c3oc3c5ccccc5ccc43)c3cccc4c3c3ccccc3n4-c3ccccc3)cccc21]

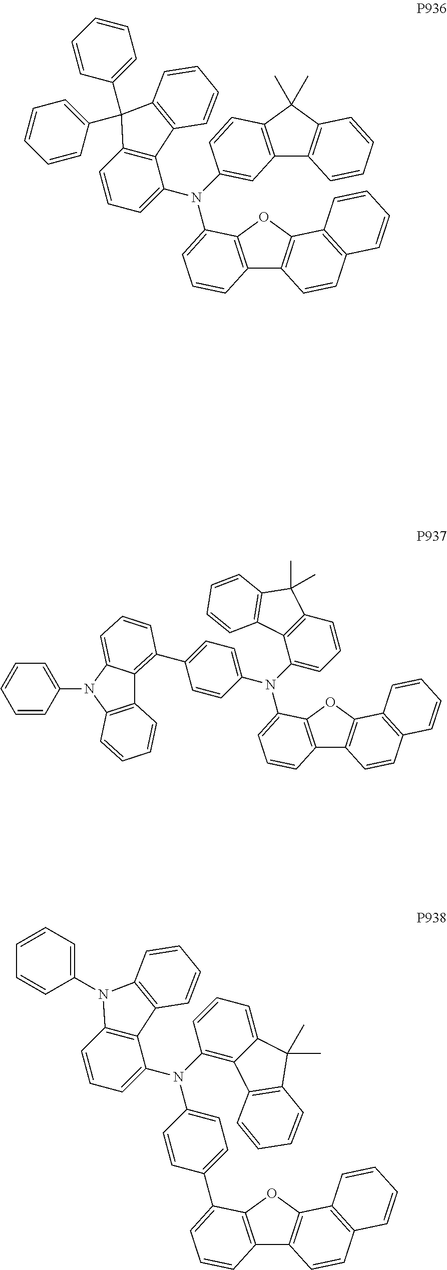 [SMILES: CC1(C)c2ccccc2-c2cc(N(c3cccc4c3-c3ccccc3C4(c3ccccc3)c3ccccc3)c3cccc4c3oc3c5ccccc5ccc43)ccc21 ; CC1(C)c2ccccc2-c2c(N(c3ccc(-c4cccc5c4c4ccccc4n5-c4ccccc4)cc3)c3cccc4c3oc3c5ccccc5ccc43)cccc21 ; CC1(C)c2ccccc2-c2c(N(c3ccc(-c4cccc5c4oc4c6ccccc6ccc54)cc3)c3cccc4c3c3ccccc3n4-c3ccccc3)cccc21]

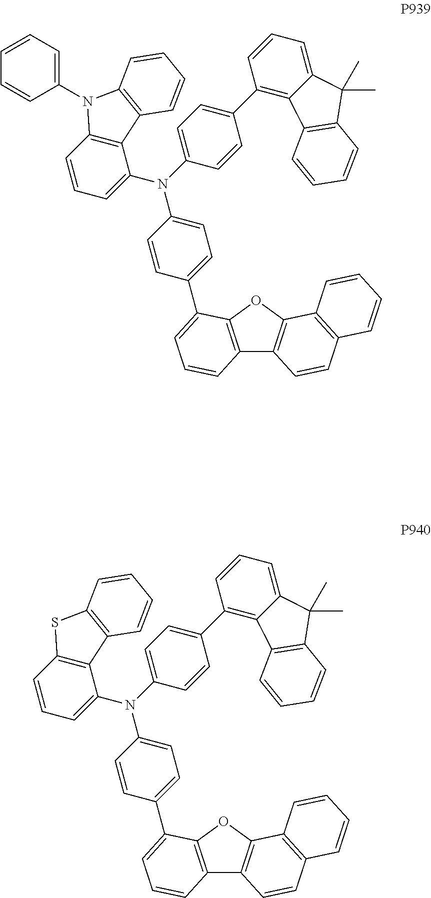 [SMILES: CC1(C)c2ccccc2-c2c(-c3ccc(N(c4ccc(-c5cccc6c5oc5c7ccccc7ccc65)cc4)c4cccc5c4c4ccccc4n5-c4ccccc4)cc3)cccc21 ; CC1(C)c2ccccc2-c2c(-c3ccc(N(c4ccc(-c5cccc6c5oc5c7ccccc7ccc65)cc4)c4cccc5sc6ccccc6c45)cc3)cccc21]

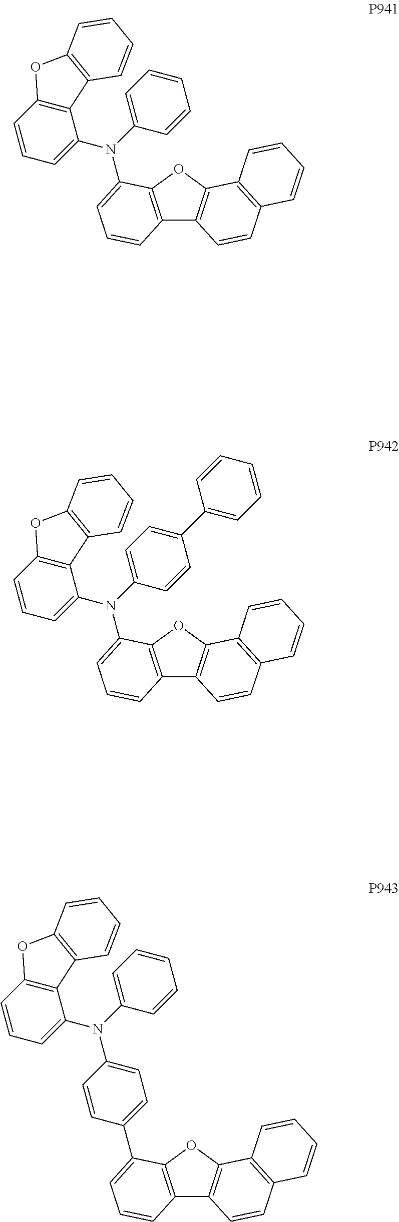 [SMILES: c1ccc(N(c2cccc3c2oc2c4ccccc4ccc32)c2cccc3oc4ccccc4c23)cc1 ; c1ccc(-c2ccc(N(c3cccc4c3oc3c5ccccc5ccc43)c3cccc4oc5ccccc5c34)cc2)cc1 ; c1ccc(N(c2ccc(-c3cccc4c3oc3c5ccccc5ccc43)cc2)c2cccc3oc4ccccc4c23)cc1]

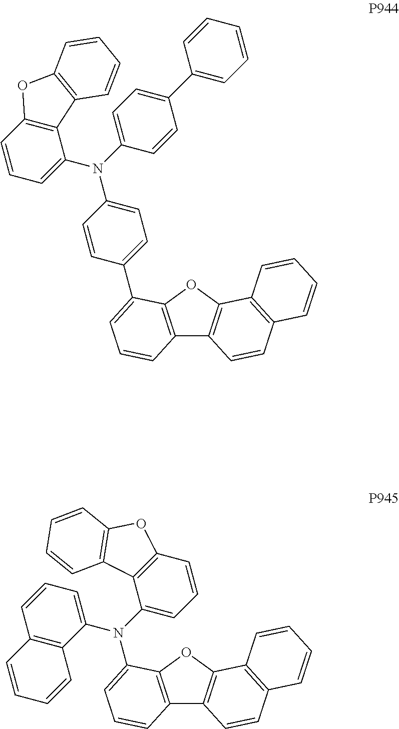 [SMILES: c1ccc(-c2ccc(N(c3ccc(-c4cccc5c4oc4c6ccccc6ccc54)cc3)c3cccc4oc5ccccc5c34)cc2)cc1 ; c1ccc2c(N(c3cccc4c3oc3c5ccccc5ccc43)c3cccc4oc5ccccc5c34)cccc2c1]

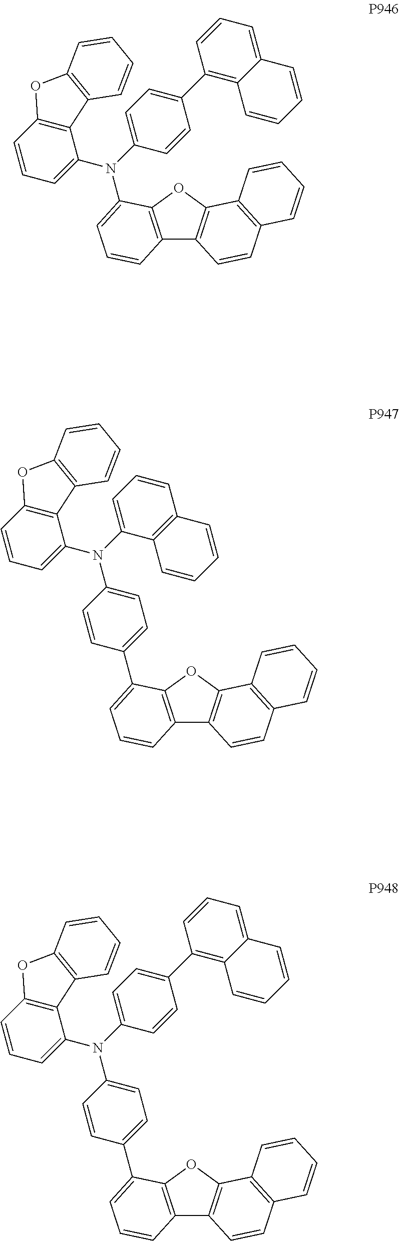 [SMILES: c1ccc2c(-c3ccc(N(c4cccc5c4oc4c6ccccc6ccc54)c4cccc5oc6ccccc6c45)cc3)cccc2c1 ; c1ccc2c(N(c3ccc(-c4cccc5c4oc4c6ccccc6ccc54)cc3)c3cccc4oc5ccccc5c34)cccc2c1 ; c1ccc2c(-c3ccc(N(c4ccc(-c5cccc6c5oc5c7ccccc7ccc65)cc4)c4cccc5oc6ccccc6c45)cc3)cccc2c1]

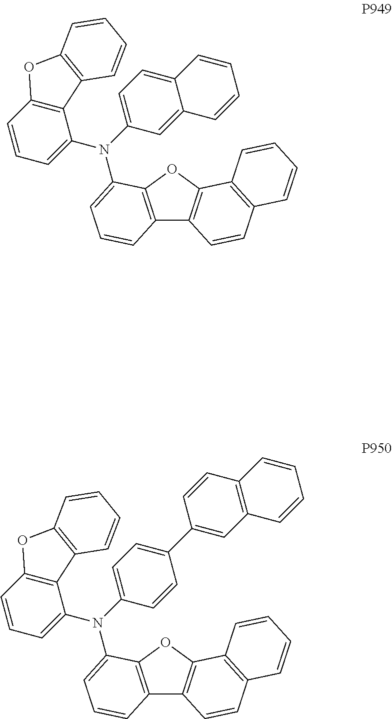 [SMILES: c1ccc2cc(N(c3cccc4c3oc3c5ccccc5ccc43)c3cccc4oc5ccccc5c34)ccc2c1 ; c1ccc2cc(-c3ccc(N(c4cccc5c4oc4c6ccccc6ccc54)c4cccc5oc6ccccc6c45)cc3)ccc2c1]

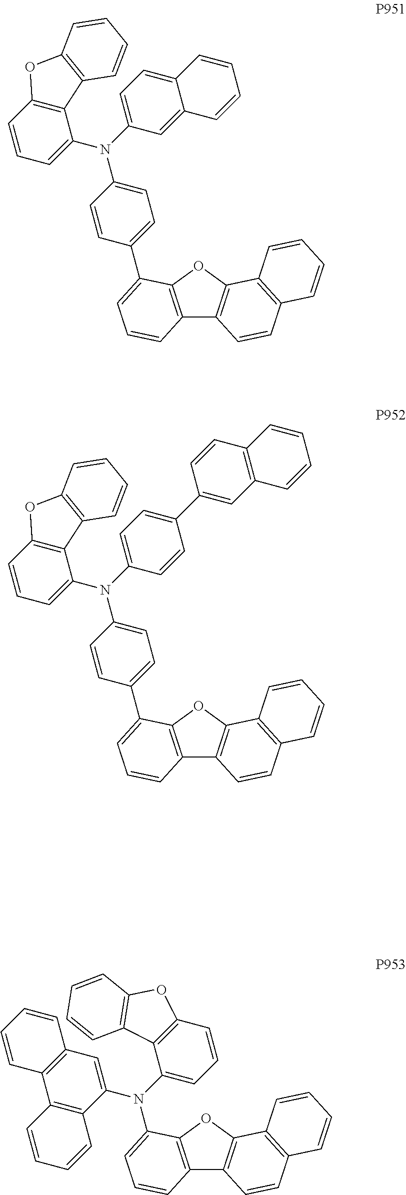 [SMILES: c1ccc2cc(N(c3ccc(-c4cccc5c4oc4c6ccccc6ccc54)cc3)c3cccc4oc5ccccc5c34)ccc2c1 ; c1ccc2cc(-c3ccc(N(c4ccc(-c5cccc6c5oc5c7ccccc7ccc65)cc4)c4cccc5oc6ccccc6c45)cc3)ccc2c1 ; c1ccc2c(c1)cc(N(c1cccc3c1oc1c4ccccc4ccc31)c1cccc3oc4ccccc4c13)c1ccccc12]

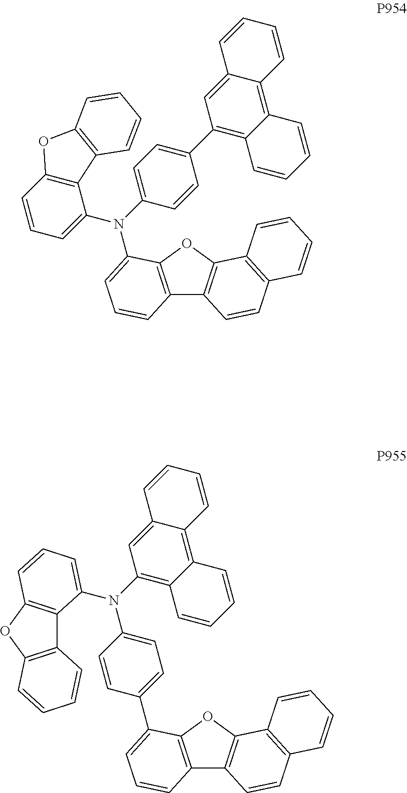 [SMILES: c1ccc2c(c1)cc(-c1ccc(N(c3cccc4c3oc3c5ccccc5ccc43)c3cccc4oc5ccccc5c34)cc1)c1ccccc12 ; c1ccc2c(c1)cc(N(c1ccc(-c3cccc4c3oc3c5ccccc5ccc43)cc1)c1cccc3oc4ccccc4c13)c1ccccc12]

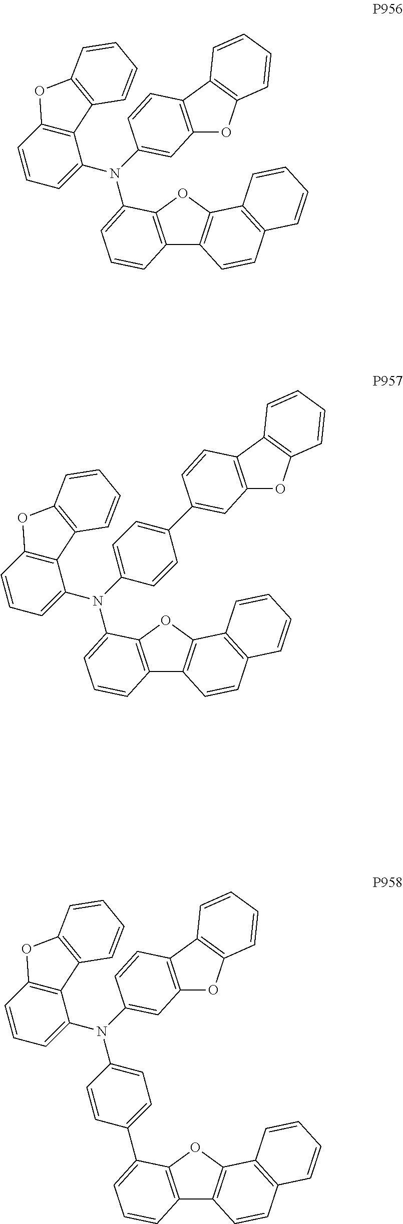 [SMILES: c1ccc2c(c1)ccc1c3cccc(N(c4ccc5c(c4)oc4ccccc45)c4cccc5oc6ccccc6c45)c3oc21 ; c1ccc2c(c1)ccc1c3cccc(N(c4ccc(-c5ccc6c(c5)oc5ccccc56)cc4)c4cccc5oc6ccccc6c45)c3oc21 ; c1ccc2c(c1)ccc1c3cccc(-c4ccc(N(c5ccc6c(c5)oc5ccccc56)c5cccc6oc7ccccc7c56)cc4)c3oc21]

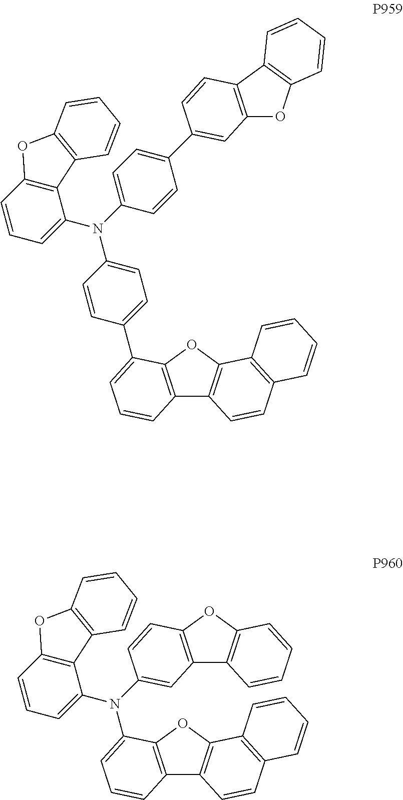 [SMILES: c1ccc2c(c1)ccc1c3cccc(-c4ccc(N(c5ccc(-c6ccc7c(c6)oc6ccccc67)cc5)c5cccc6oc7ccccc7c56)cc4)c3oc21 ; c1ccc2c(c1)ccc1c3cccc(N(c4ccc5oc6ccccc6c5c4)c4cccc5oc6ccccc6c45)c3oc21]

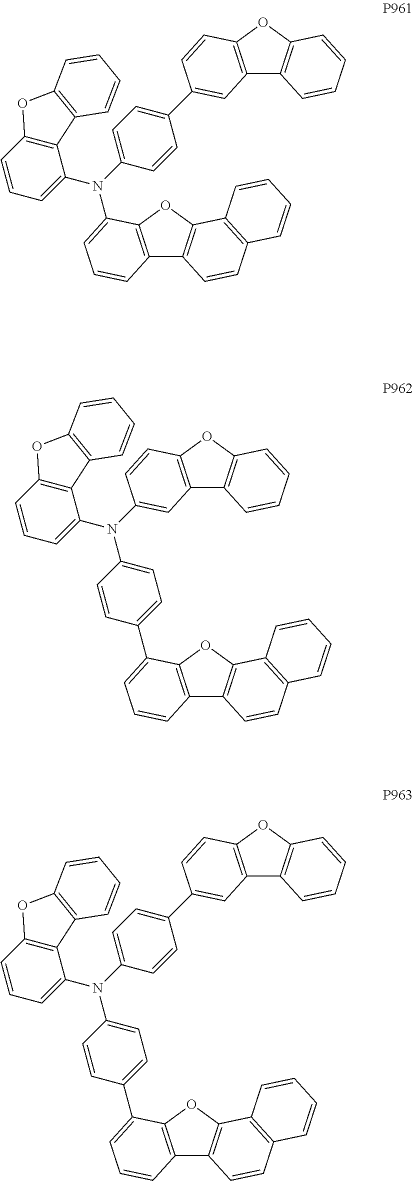 [SMILES: c1ccc2c(c1)ccc1c3cccc(N(c4ccc(-c5ccc6oc7ccccc7c6c5)cc4)c4cccc5oc6ccccc6c45)c3oc21 ; c1ccc2c(c1)ccc1c3cccc(-c4ccc(N(c5ccc6oc7ccccc7c6c5)c5cccc6oc7ccccc7c56)cc4)c3oc21 ; c1ccc2c(c1)ccc1c3cccc(-c4ccc(N(c5ccc(-c6ccc7oc8ccccc8c7c6)cc5)c5cccc6oc7ccccc7c56)cc4)c3oc21]

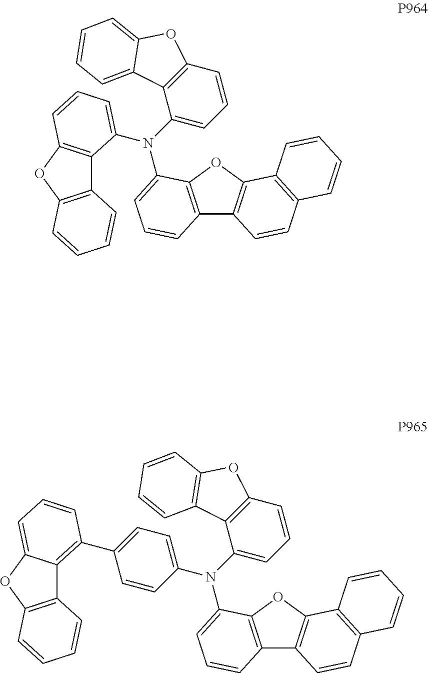 [SMILES: c1ccc2c(c1)ccc1c3cccc(N(c4cccc5oc6ccccc6c45)c4cccc5oc6ccccc6c45)c3oc21 ; c1ccc2c(c1)ccc1c3cccc(N(c4ccc(-c5cccc6oc7ccccc7c56)cc4)c4cccc5oc6ccccc6c45)c3oc21]

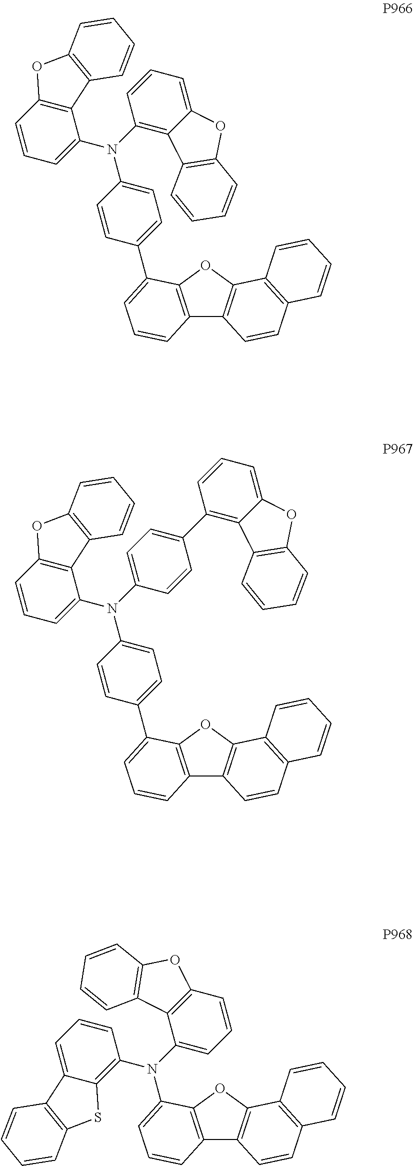 [SMILES: c1ccc2c(c1)ccc1c3cccc(-c4ccc(N(c5cccc6oc7ccccc7c56)c5cccc6oc7ccccc7c56)cc4)c3oc21 ; c1ccc2c(c1)ccc1c3cccc(-c4ccc(N(c5ccc(-c6cccc7oc8ccccc8c67)cc5)c5cccc6oc7ccccc7c56)cc4)c3oc21 ; c1ccc2c(c1)ccc1c3cccc(N(c4cccc5c4sc4ccccc45)c4cccc5oc6ccccc6c45)c3oc21]

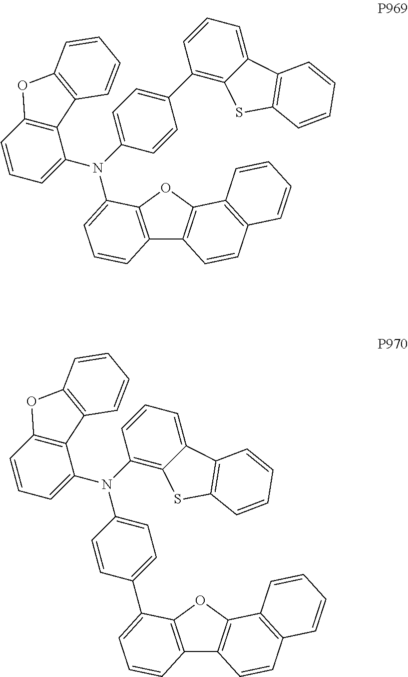 [SMILES: c1ccc2c(c1)ccc1c3cccc(N(c4ccc(-c5cccc6c5sc5ccccc56)cc4)c4cccc5oc6ccccc6c45)c3oc21 ; c1ccc2c(c1)ccc1c3cccc(-c4ccc(N(c5cccc6c5sc5ccccc56)c5cccc6oc7ccccc7c56)cc4)c3oc21]

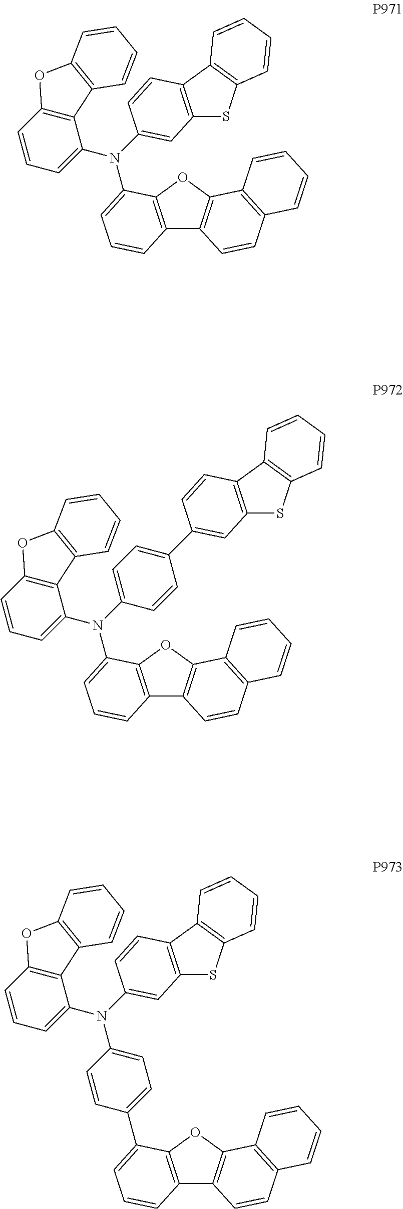 [SMILES: c1ccc2c(c1)ccc1c3cccc(N(c4ccc5c(c4)sc4ccccc45)c4cccc5oc6ccccc6c45)c3oc21 ; c1ccc2c(c1)ccc1c3cccc(N(c4ccc(-c5ccc6c(c5)sc5ccccc56)cc4)c4cccc5oc6ccccc6c45)c3oc21 ; c1ccc2c(c1)ccc1c3cccc(-c4ccc(N(c5ccc6c(c5)sc5ccccc56)c5cccc6oc7ccccc7c56)cc4)c3oc21]

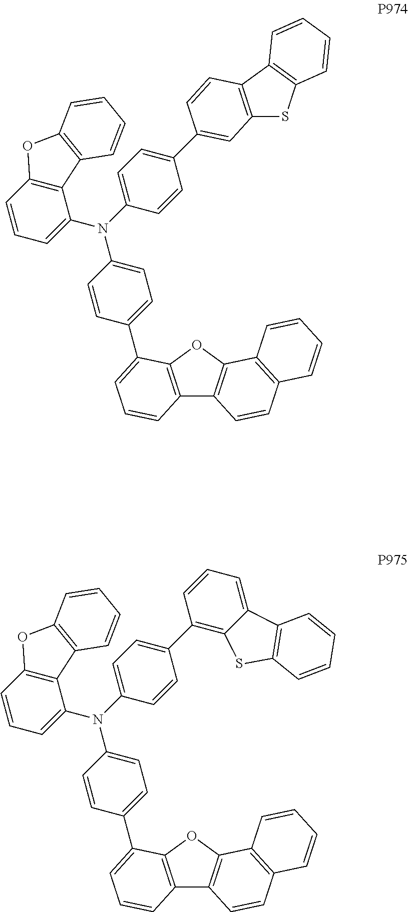 [SMILES: c1ccc2c(c1)ccc1c3cccc(-c4ccc(N(c5ccc(-c6ccc7c(c6)sc6ccccc67)cc5)c5cccc6oc7ccccc7c56)cc4)c3oc21 ; c1ccc2c(c1)ccc1c3cccc(-c4ccc(N(c5ccc(-c6cccc7c6sc6ccccc67)cc5)c5cccc6oc7ccccc7c56)cc4)c3oc21]

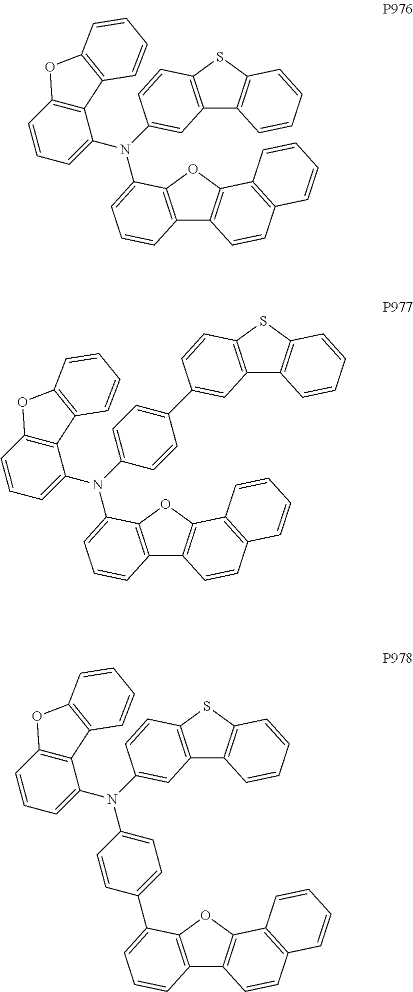 [SMILES: c1ccc2c(c1)ccc1c3cccc(N(c4ccc5sc6ccccc6c5c4)c4cccc5oc6ccccc6c45)c3oc21 ; c1ccc2c(c1)ccc1c3cccc(N(c4ccc(-c5ccc6sc7ccccc7c6c5)cc4)c4cccc5oc6ccccc6c45)c3oc21 ; c1ccc2c(c1)ccc1c3cccc(-c4ccc(N(c5ccc6sc7ccccc7c6c5)c5cccc6oc7ccccc7c56)cc4)c3oc21]

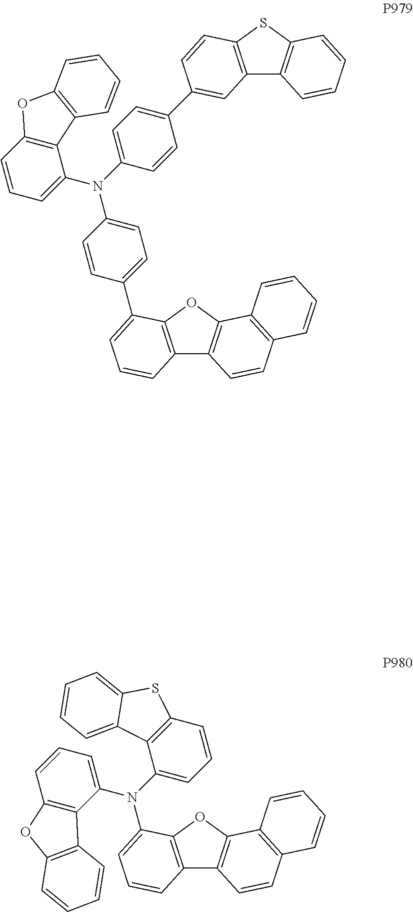 [SMILES: c1ccc2c(c1)ccc1c3cccc(-c4ccc(N(c5ccc(-c6ccc7sc8ccccc8c7c6)cc5)c5cccc6oc7ccccc7c56)cc4)c3oc21 ; c1ccc2c(c1)ccc1c3cccc(N(c4cccc5oc6ccccc6c45)c4cccc5sc6ccccc6c45)c3oc21]

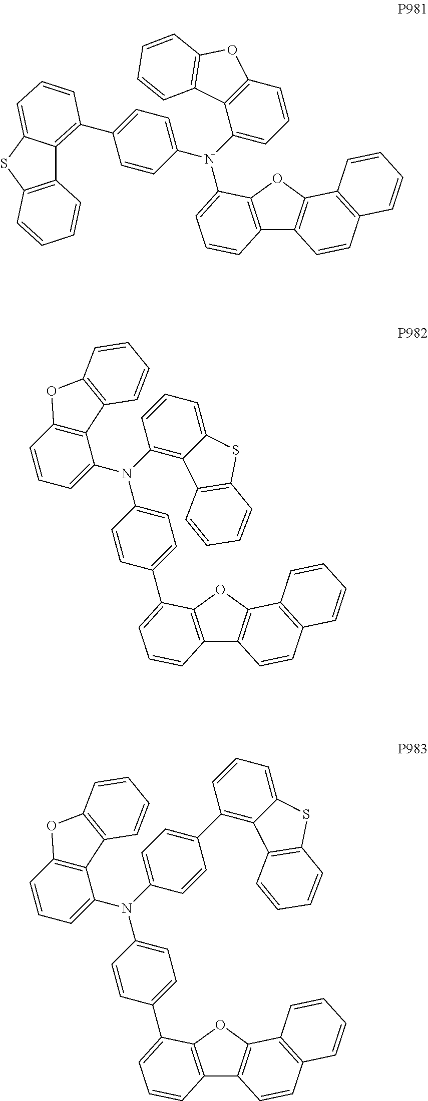 [SMILES: c1ccc2c(c1)ccc1c3cccc(N(c4ccc(-c5cccc6sc7ccccc7c56)cc4)c4cccc5oc6ccccc6c45)c3oc21 ; c1ccc2c(c1)ccc1c3cccc(-c4ccc(N(c5cccc6oc7ccccc7c56)c5cccc6sc7ccccc7c56)cc4)c3oc21 ; c1ccc2c(c1)ccc1c3cccc(-c4ccc(N(c5ccc(-c6cccc7sc8ccccc8c67)cc5)c5cccc6oc7ccccc7c56)cc4)c3oc21]

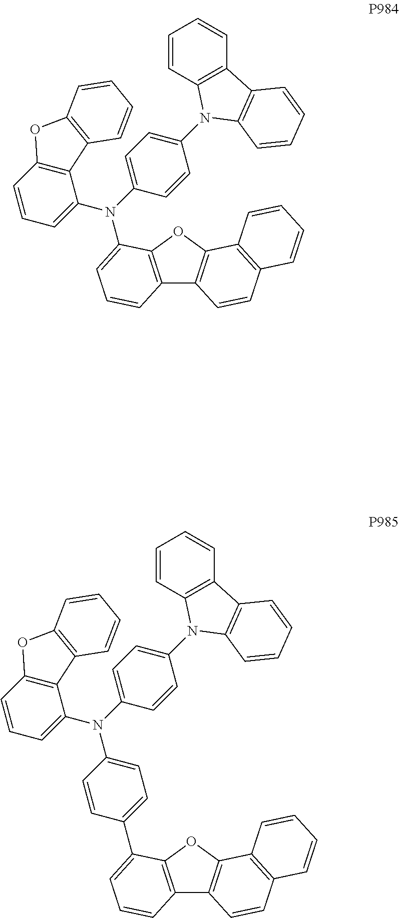 [SMILES: c1ccc2c(c1)ccc1c3cccc(N(c4ccc(-n5c6ccccc6c6ccccc65)cc4)c4cccc5oc6ccccc6c45)c3oc21 ; c1ccc2c(c1)ccc1c3cccc(-c4ccc(N(c5ccc(-n6c7ccccc7c7ccccc76)cc5)c5cccc6oc7ccccc7c56)cc4)c3oc21]

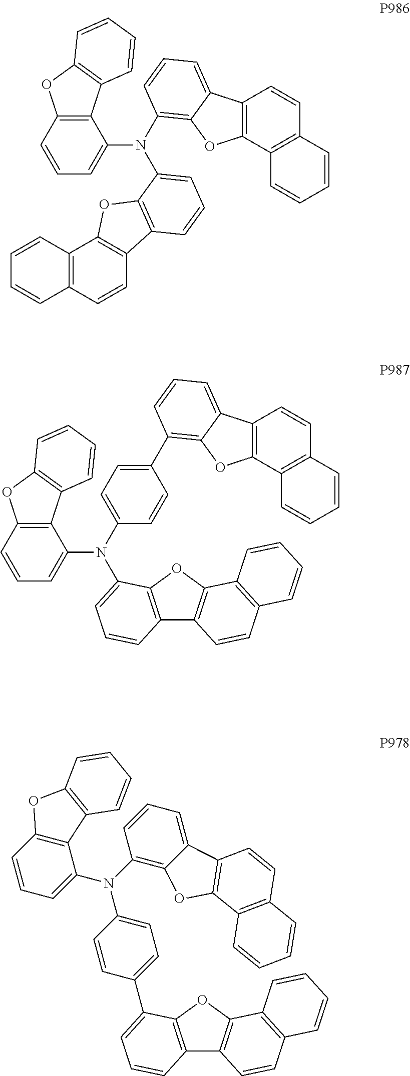 [SMILES: c1ccc2c(c1)ccc1c3cccc(N(c4cccc5c4oc4c6ccccc6ccc54)c4cccc5oc6ccccc6c45)c3oc21 ; c1ccc2c(c1)ccc1c3cccc(-c4ccc(N(c5cccc6c5oc5c7ccccc7ccc65)c5cccc6oc7ccccc7c56)cc4)c3oc21 ; c1ccc2c(c1)ccc1c3cccc(-c4ccc(N(c5cccc6c5oc5c7ccccc7ccc65)c5cccc6oc7ccccc7c56)cc4)c3oc21]

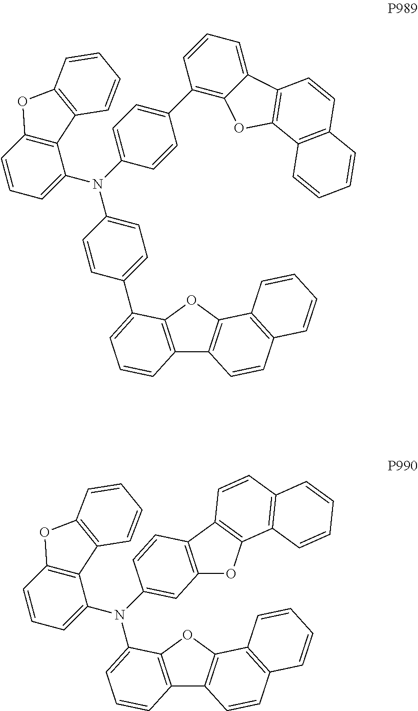 [SMILES: c1ccc2c(c1)ccc1c3cccc(-c4ccc(N(c5ccc(-c6cccc7c6oc6c8ccccc8ccc76)cc5)c5cccc6oc7ccccc7c56)cc4)c3oc21 ; c1ccc2c(c1)ccc1c3ccc(N(c4cccc5c4oc4c6ccccc6ccc54)c4cccc5oc6ccccc6c45)cc3oc21]

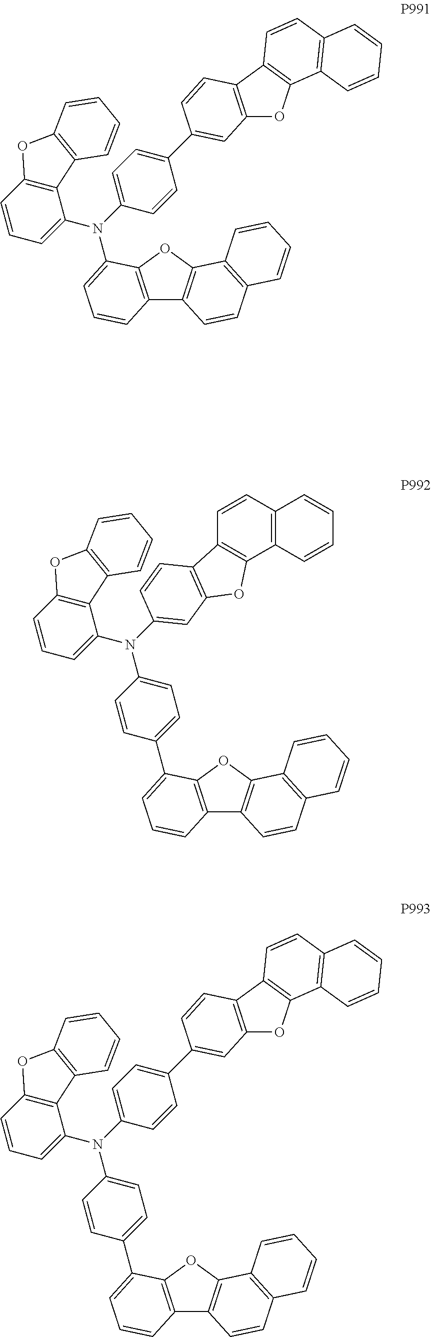 [SMILES: c1ccc2c(c1)ccc1c3ccc(-c4ccc(N(c5cccc6c5oc5c7ccccc7ccc65)c5cccc6oc7ccccc7c56)cc4)cc3oc21 ; c1ccc2c(c1)ccc1c3ccc(N(c4ccc(-c5cccc6c5oc5c7ccccc7ccc65)cc4)c4cccc5oc6ccccc6c45)cc3oc21 ; c1ccc2c(c1)ccc1c3ccc(-c4ccc(N(c5ccc(-c6cccc7c6oc6c8ccccc8ccc76)cc5)c5cccc6oc7ccccc7c56)cc4)cc3oc21]

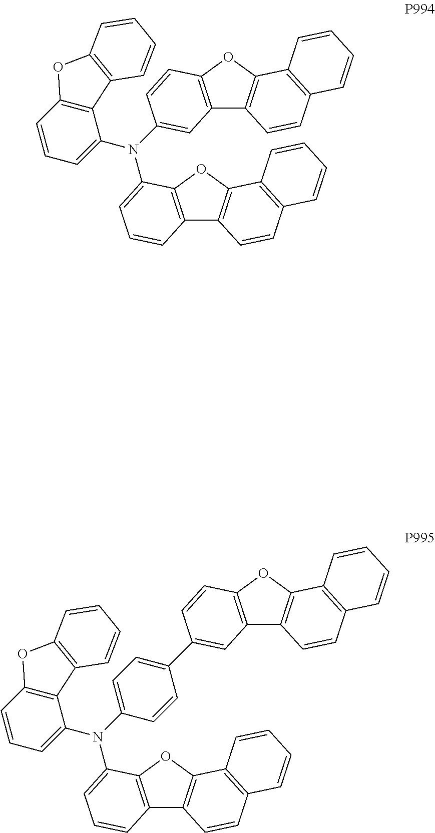 [SMILES: c1ccc2c(c1)ccc1c3cc(N(c4cccc5c4oc4c6ccccc6ccc54)c4cccc5oc6ccccc6c45)ccc3oc21 ; c1ccc2c(c1)ccc1c3cc(-c4ccc(N(c5cccc6c5oc5c7ccccc7ccc65)c5cccc6oc7ccccc7c56)cc4)ccc3oc21]

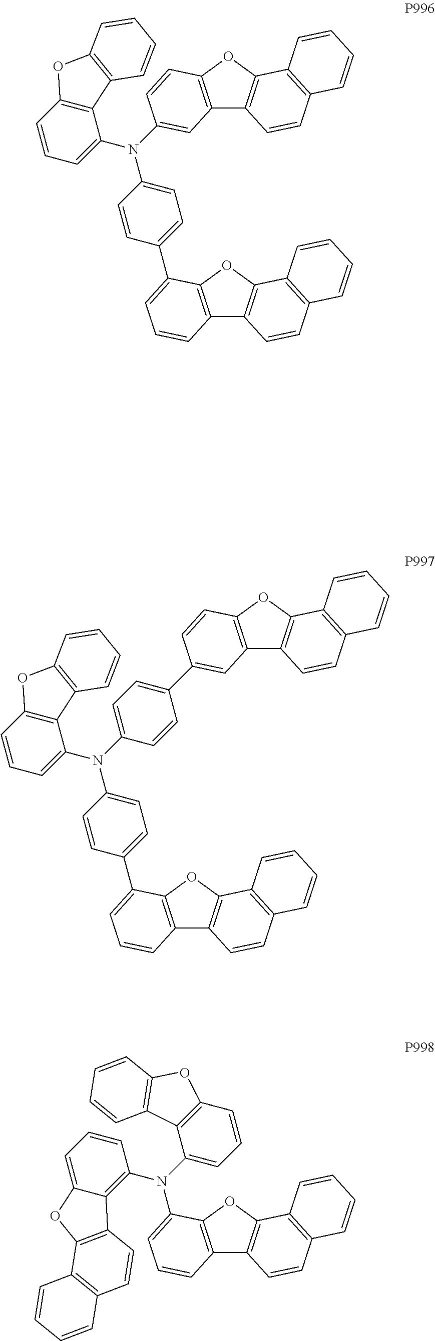 [SMILES: c1ccc2c(c1)ccc1c3cc(N(c4ccc(-c5cccc6c5oc5c7ccccc7ccc65)cc4)c4cccc5oc6ccccc6c45)ccc3oc21 ; c1ccc2c(c1)ccc1c3cc(-c4ccc(N(c5ccc(-c6cccc7c6oc6c8ccccc8ccc76)cc5)c5cccc6oc7ccccc7c56)cc4)ccc3oc21 ; c1ccc2c(c1)ccc1c3cccc(N(c4cccc5oc6ccccc6c45)c4cccc5oc6c7ccccc7ccc6c45)c3oc21]

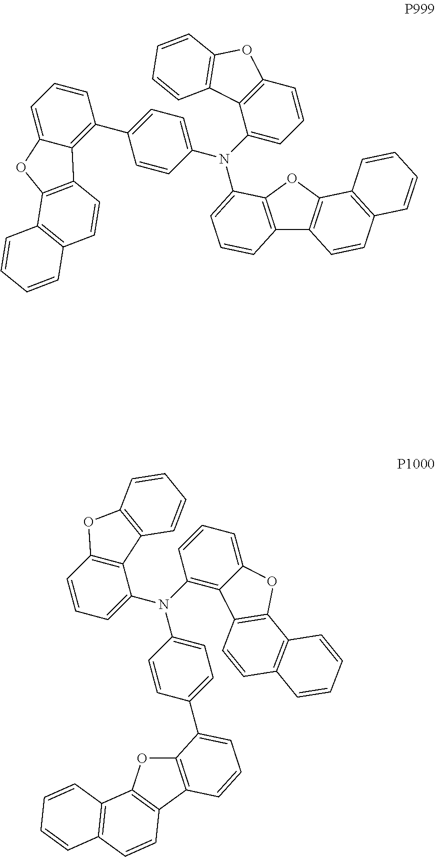 [SMILES: c1ccc2c(c1)ccc1c3cccc(N(c4ccc(-c5cccc6oc7c8ccccc8ccc7c56)cc4)c4cccc5oc6ccccc6c45)c3oc21 ; c1ccc2c(c1)ccc1c3cccc(-c4ccc(N(c5cccc6oc7ccccc7c56)c5cccc6oc7c8ccccc8ccc7c56)cc4)c3oc21]

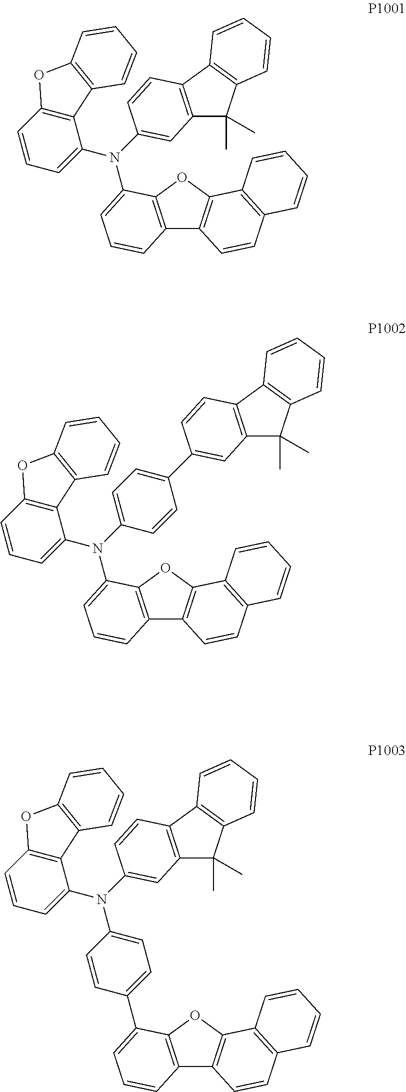 [SMILES: CC1(C)c2ccccc2-c2ccc(N(c3cccc4c3oc3c5ccccc5ccc43)c3cccc4oc5ccccc5c34)cc21 ; CC1(C)c2ccccc2-c2ccc(-c3ccc(N(c4cccc5c4oc4c6ccccc6ccc54)c4cccc5oc6ccccc6c45)cc3)cc21 ; CC1(C)c2ccccc2-c2ccc(N(c3ccc(-c4cccc5c4oc4c6ccccc6ccc54)cc3)c3cccc4oc5ccccc5c34)cc21]

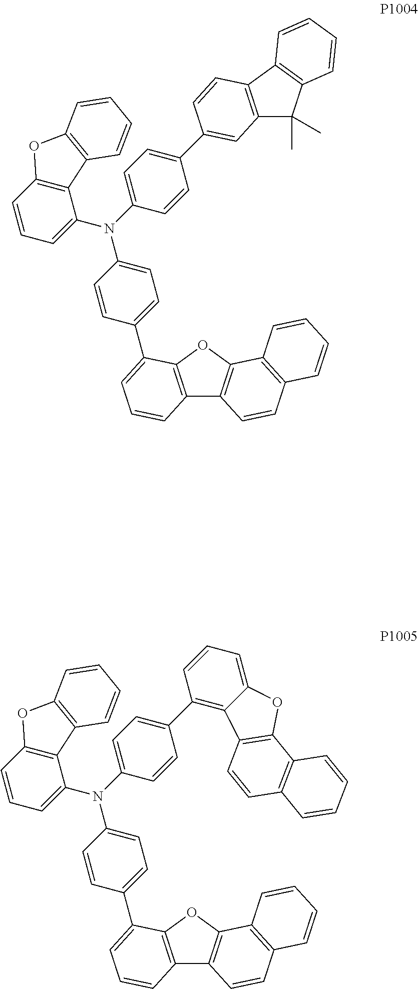 [SMILES: CC1(C)c2ccccc2-c2ccc(-c3ccc(N(c4ccc(-c5cccc6c5oc5c7ccccc7ccc65)cc4)c4cccc5oc6ccccc6c45)cc3)cc21 ; c1ccc2c(c1)ccc1c3cccc(-c4ccc(N(c5ccc(-c6cccc7oc8c9ccccc9ccc8c67)cc5)c5cccc6oc7ccccc7c56)cc4)c3oc21]

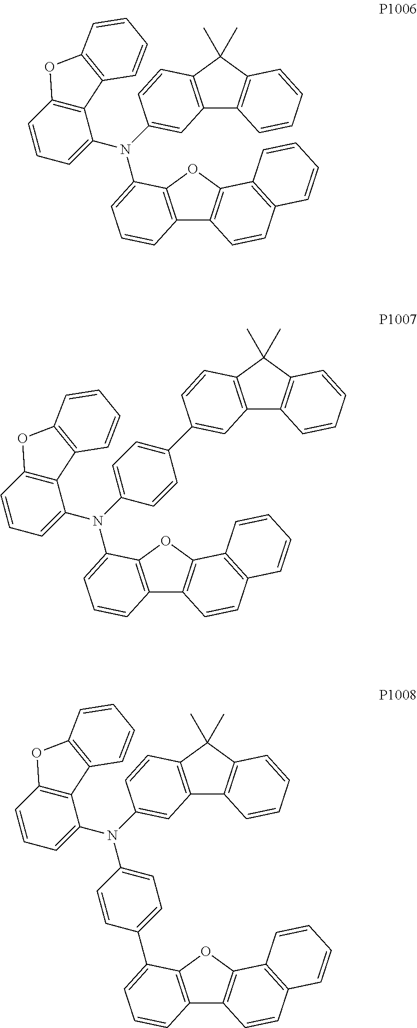 [SMILES: CC1(C)c2ccccc2-c2cc(N(c3cccc4c3oc3c5ccccc5ccc43)c3cccc4oc5ccccc5c34)ccc21 ; CC1(C)c2ccccc2-c2cc(-c3ccc(N(c4cccc5c4oc4c6ccccc6ccc54)c4cccc5oc6ccccc6c45)cc3)ccc21 ; CC1(C)c2ccccc2-c2cc(N(c3ccc(-c4cccc5c4oc4c6ccccc6ccc54)cc3)c3cccc4oc5ccccc5c34)ccc21]

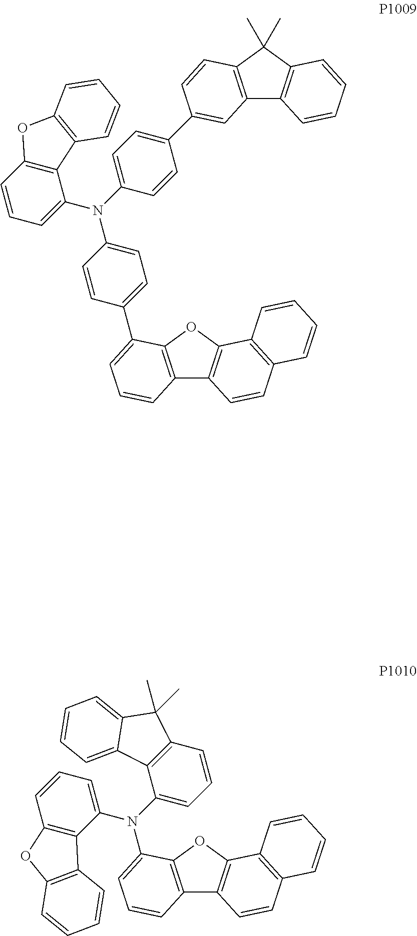 [SMILES: CC1(C)c2ccccc2-c2cc(-c3ccc(N(c4ccc(-c5cccc6c5oc5c7ccccc7ccc65)cc4)c4cccc5oc6ccccc6c45)cc3)ccc21 ; CC1(C)c2ccccc2-c2c(N(c3cccc4c3oc3c5ccccc5ccc43)c3cccc4oc5ccccc5c34)cccc21]

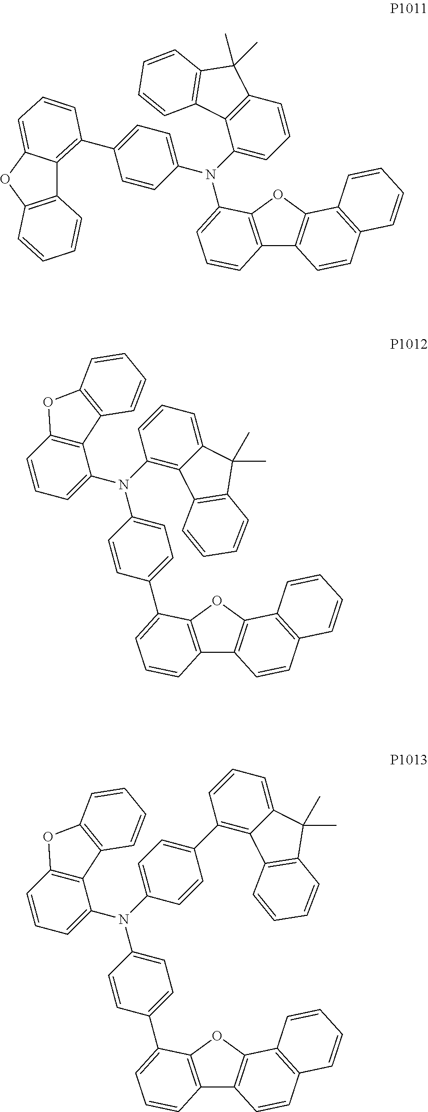 [SMILES: CC1(C)c2ccccc2-c2c(N(c3ccc(-c4cccc5oc6ccccc6c45)cc3)c3cccc4c3oc3c5ccccc5ccc43)cccc21 ; CC1(C)c2ccccc2-c2c(N(c3ccc(-c4cccc5c4oc4c6ccccc6ccc54)cc3)c3cccc4oc5ccccc5c34)cccc21 ; CC1(C)c2ccccc2-c2c(-c3ccc(N(c4ccc(-c5cccc6c5oc5c7ccccc7ccc65)cc4)c4cccc5oc6ccccc6c45)cc3)cccc21]

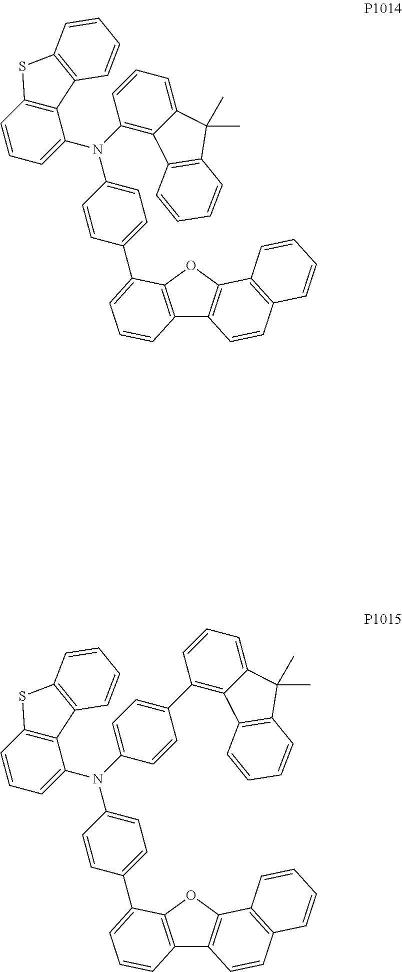 [SMILES: CC1(C)c2ccccc2-c2c(N(c3ccc(-c4cccc5c4oc4c6ccccc6ccc54)cc3)c3cccc4sc5ccccc5c34)cccc21 ; CC1(C)c2ccccc2-c2c(-c3ccc(N(c4ccc(-c5cccc6c5oc5c7ccccc7ccc65)cc4)c4cccc5sc6ccccc6c45)cc3)cccc21]

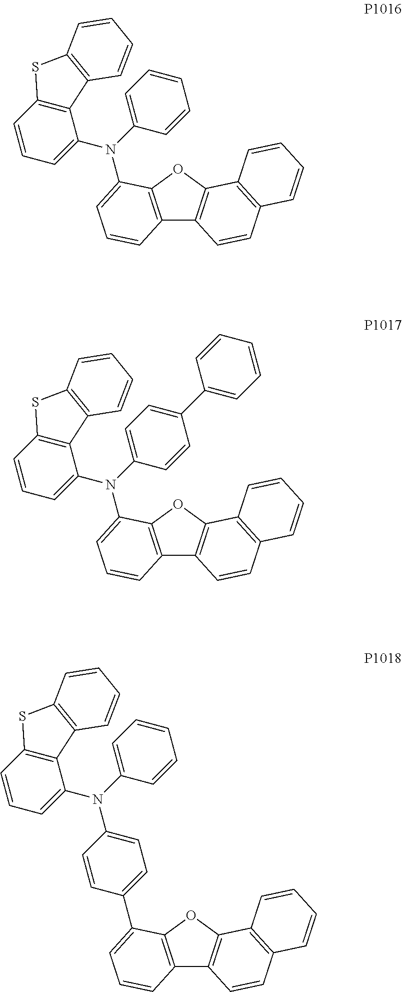 [SMILES: c1ccc(N(c2cccc3c2oc2c4ccccc4ccc32)c2cccc3sc4ccccc4c23)cc1 ; c1ccc(-c2ccc(N(c3cccc4c3oc3c5ccccc5ccc43)c3cccc4sc5ccccc5c34)cc2)cc1 ; c1ccc(N(c2ccc(-c3cccc4c3oc3c5ccccc5ccc43)cc2)c2cccc3sc4ccccc4c23)cc1]

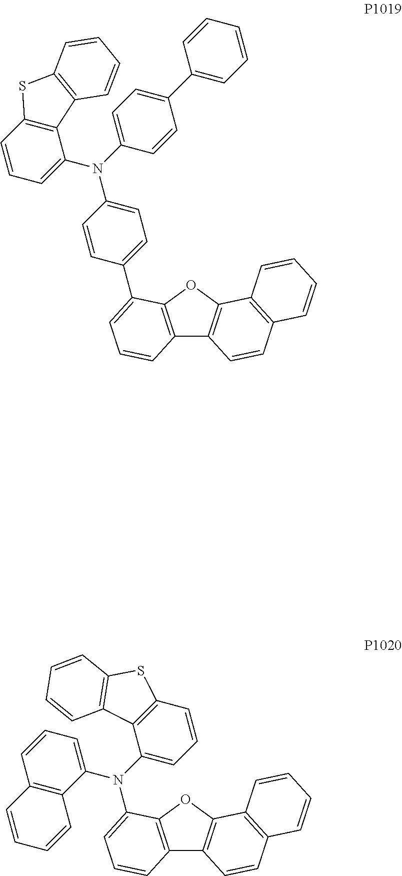 [SMILES: c1ccc(-c2ccc(N(c3ccc(-c4cccc5c4oc4c6ccccc6ccc54)cc3)c3cccc4sc5ccccc5c34)cc2)cc1 ; c1ccc2c(N(c3cccc4c3oc3c5ccccc5ccc43)c3cccc4sc5ccccc5c34)cccc2c1]

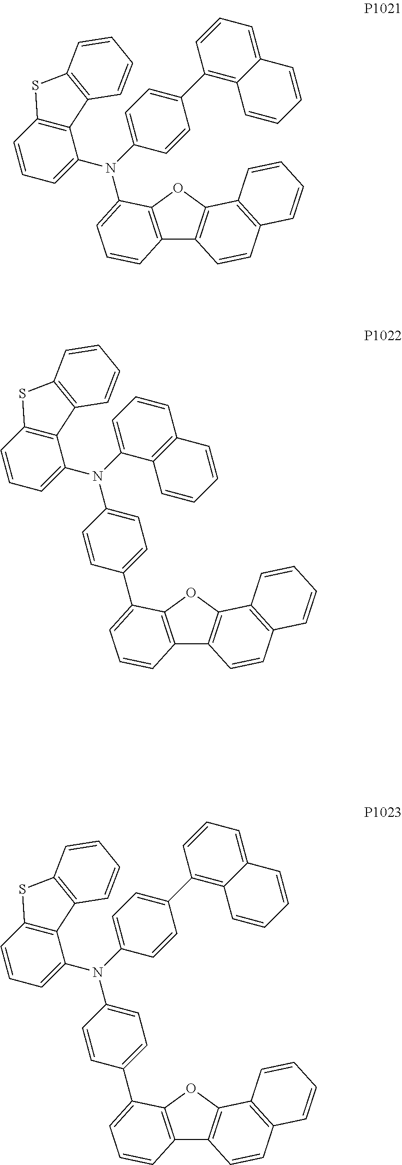 [SMILES: c1ccc2c(-c3ccc(N(c4cccc5c4oc4c6ccccc6ccc54)c4cccc5sc6ccccc6c45)cc3)cccc2c1 ; c1ccc2c(N(c3ccc(-c4cccc5c4oc4c6ccccc6ccc54)cc3)c3cccc4sc5ccccc5c34)cccc2c1 ; c1ccc2c(-c3ccc(N(c4ccc(-c5cccc6c5oc5c7ccccc7ccc65)cc4)c4cccc5sc6ccccc6c45)cc3)cccc2c1]

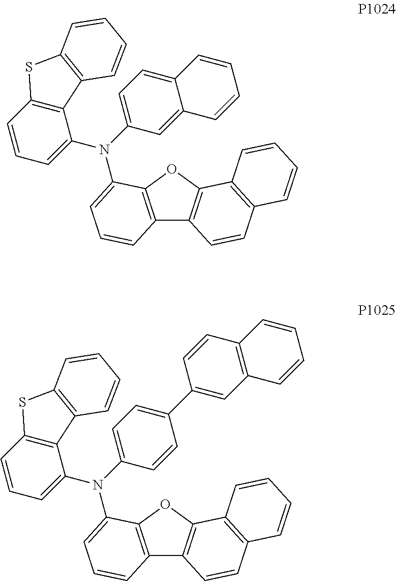 [SMILES: c1ccc2cc(N(c3cccc4c3oc3c5ccccc5ccc43)c3cccc4sc5ccccc5c34)ccc2c1 ; c1ccc2cc(-c3ccc(N(c4cccc5c4oc4c6ccccc6ccc54)c4cccc5sc6ccccc6c45)cc3)ccc2c1]

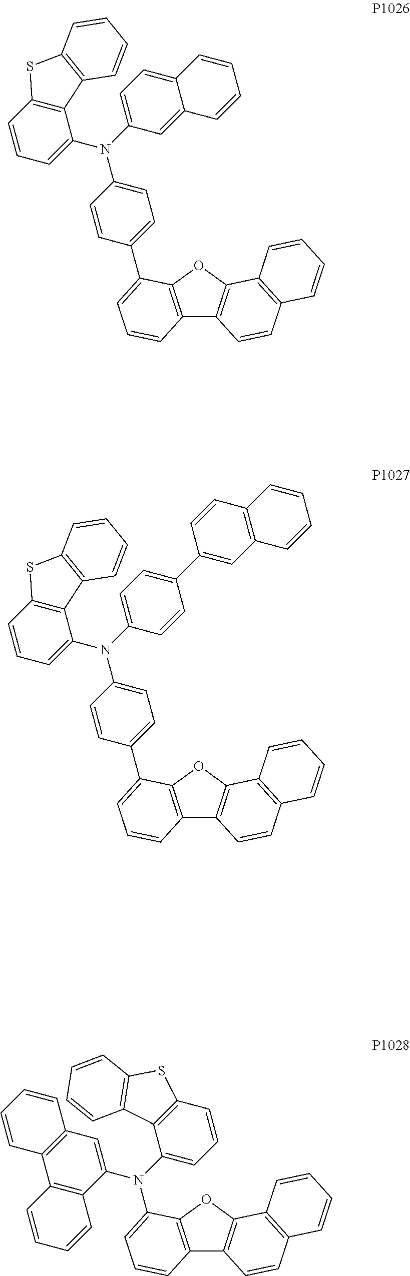 [SMILES: c1ccc2cc(N(c3ccc(-c4cccc5c4oc4c6ccccc6ccc54)cc3)c3cccc4sc5ccccc5c34)ccc2c1 ; c1ccc2cc(-c3ccc(N(c4ccc(-c5cccc6c5oc5c7ccccc7ccc65)cc4)c4cccc5sc6ccccc6c45)cc3)ccc2c1 ; c1ccc2c(c1)cc(N(c1cccc3c1oc1c4ccccc4ccc31)c1cccc3sc4ccccc4c13)c1ccccc12]

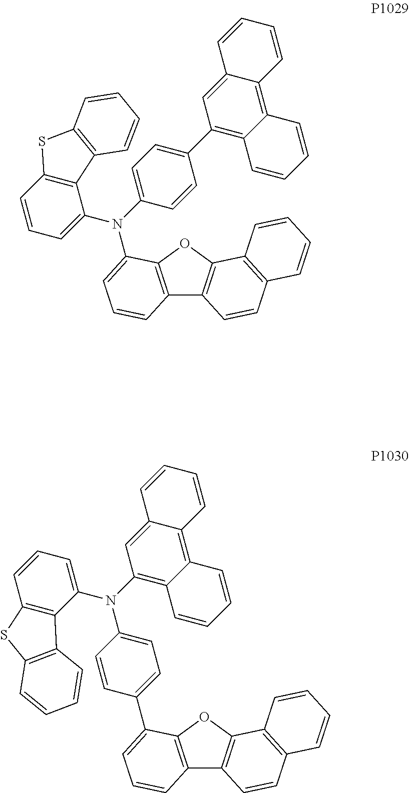 [SMILES: c1ccc2c(c1)cc(-c1ccc(N(c3cccc4c3oc3c5ccccc5ccc43)c3cccc4sc5ccccc5c34)cc1)c1ccccc12 ; c1ccc2c(c1)cc(N(c1ccc(-c3cccc4c3oc3c5ccccc5ccc43)cc1)c1cccc3sc4ccccc4c13)c1ccccc12]

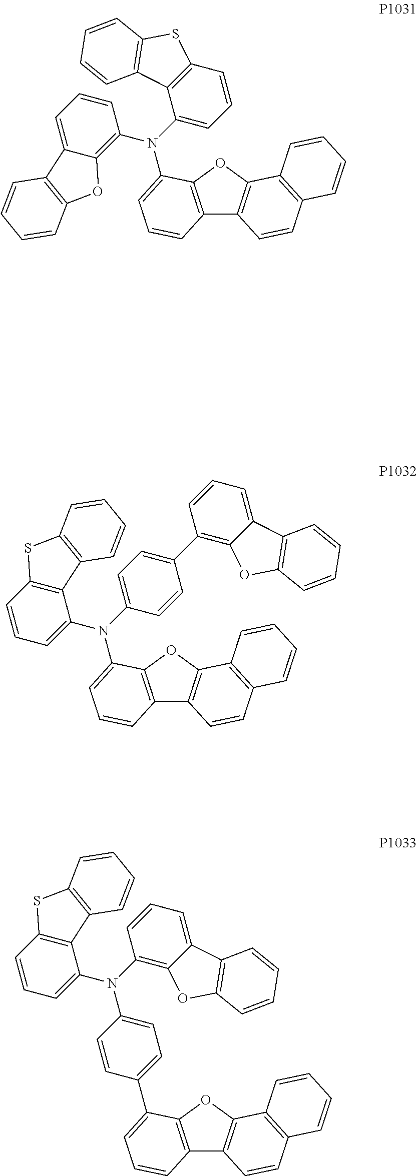 [SMILES: c1ccc2c(c1)ccc1c3cccc(N(c4cccc5c4oc4ccccc45)c4cccc5sc6ccccc6c45)c3oc21 ; c1ccc2c(c1)ccc1c3cccc(N(c4ccc(-c5cccc6c5oc5ccccc56)cc4)c4cccc5sc6ccccc6c45)c3oc21 ; c1ccc2c(c1)ccc1c3cccc(-c4ccc(N(c5cccc6c5oc5ccccc56)c5cccc6sc7ccccc7c56)cc4)c3oc21]

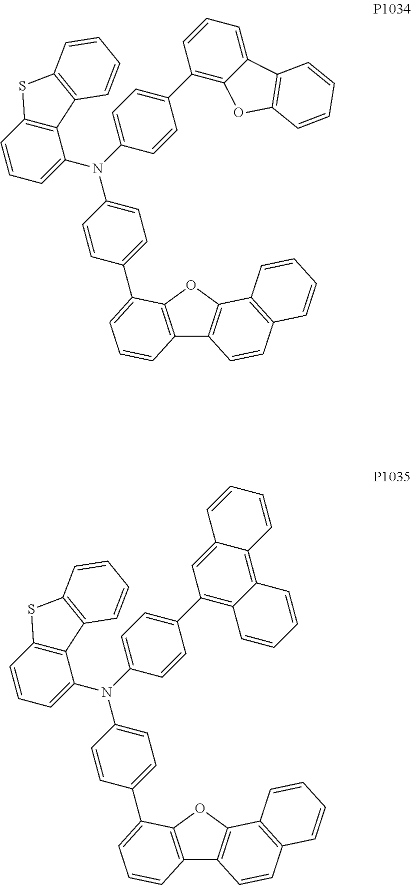 [SMILES: c1ccc2c(c1)ccc1c3cccc(-c4ccc(N(c5ccc(-c6cccc7c6oc6ccccc67)cc5)c5cccc6sc7ccccc7c56)cc4)c3oc21 ; c1ccc2c(c1)cc(-c1ccc(N(c3ccc(-c4cccc5c4oc4c6ccccc6ccc54)cc3)c3cccc4sc5ccccc5c34)cc1)c1ccccc12]

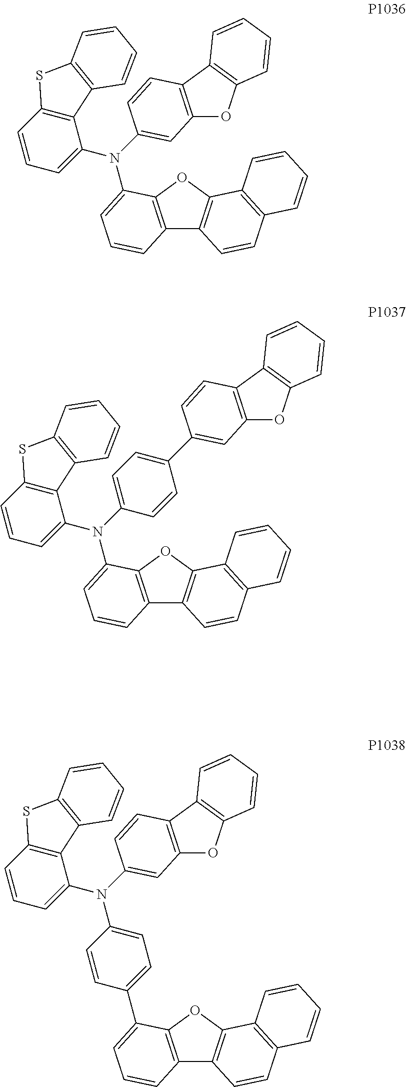 [SMILES: c1ccc2c(c1)ccc1c3cccc(N(c4ccc5c(c4)oc4ccccc45)c4cccc5sc6ccccc6c45)c3oc21 ; c1ccc2c(c1)ccc1c3cccc(N(c4ccc(-c5ccc6c(c5)oc5ccccc56)cc4)c4cccc5sc6ccccc6c45)c3oc21 ; c1ccc2c(c1)ccc1c3cccc(-c4ccc(N(c5ccc6c(c5)oc5ccccc56)c5cccc6sc7ccccc7c56)cc4)c3oc21]

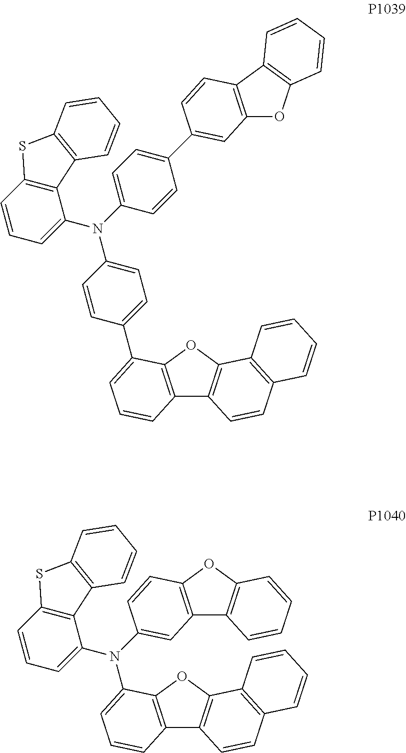 [SMILES: c1ccc2c(c1)ccc1c3cccc(-c4ccc(N(c5ccc(-c6ccc7c(c6)oc6ccccc67)cc5)c5cccc6sc7ccccc7c56)cc4)c3oc21 ; c1ccc2c(c1)ccc1c3cccc(N(c4ccc5oc6ccccc6c5c4)c4cccc5sc6ccccc6c45)c3oc21]

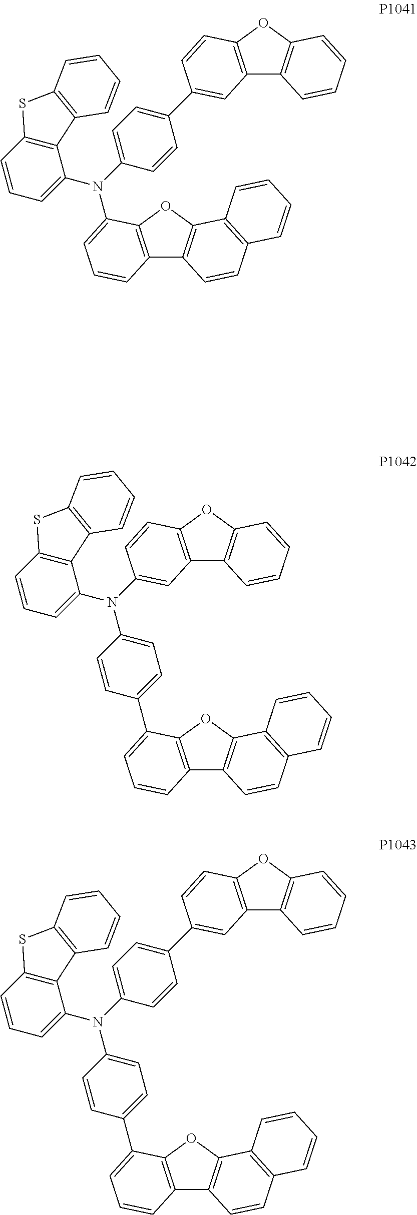 [SMILES: c1ccc2c(c1)ccc1c3cccc(N(c4ccc(-c5ccc6oc7ccccc7c6c5)cc4)c4cccc5sc6ccccc6c45)c3oc21 ; c1ccc2c(c1)ccc1c3cccc(-c4ccc(N(c5ccc6oc7ccccc7c6c5)c5cccc6sc7ccccc7c56)cc4)c3oc21 ; c1ccc2c(c1)ccc1c3cccc(-c4ccc(N(c5ccc(-c6ccc7oc8ccccc8c7c6)cc5)c5cccc6sc7ccccc7c56)cc4)c3oc21]

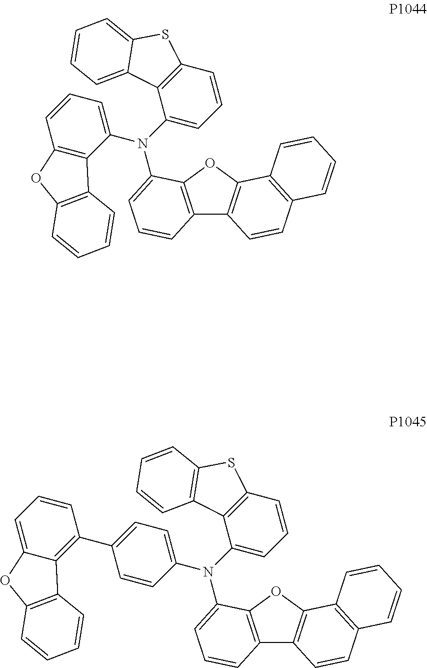 [SMILES: c1ccc2c(c1)ccc1c3cccc(N(c4cccc5oc6ccccc6c45)c4cccc5sc6ccccc6c45)c3oc21 ; c1ccc2c(c1)ccc1c3cccc(N(c4ccc(-c5cccc6oc7ccccc7c56)cc4)c4cccc5sc6ccccc6c45)c3oc21]

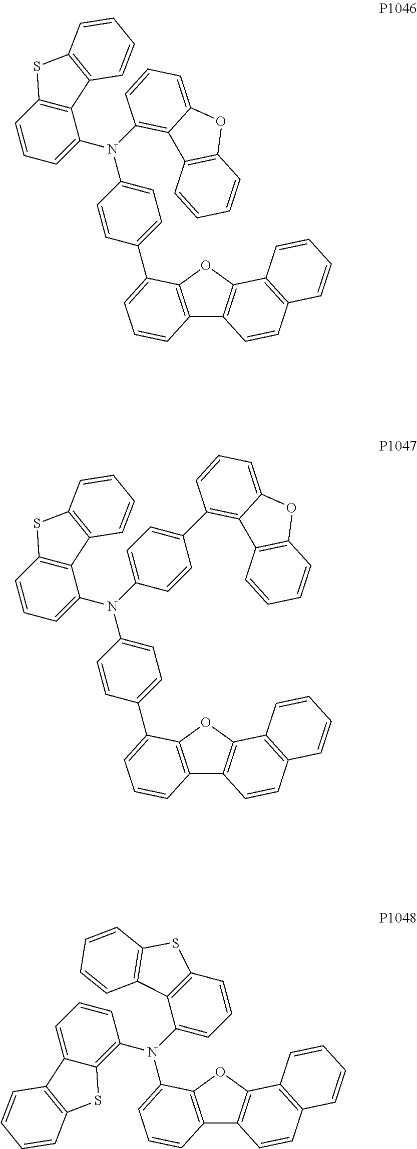 [SMILES: c1ccc2c(c1)ccc1c3cccc(-c4ccc(N(c5cccc6oc7ccccc7c56)c5cccc6sc7ccccc7c56)cc4)c3oc21 ; c1ccc2c(c1)ccc1c3cccc(-c4ccc(N(c5ccc(-c6cccc7oc8ccccc8c67)cc5)c5cccc6sc7ccccc7c56)cc4)c3oc21 ; c1ccc2c(c1)ccc1c3cccc(N(c4cccc5c4sc4ccccc45)c4cccc5sc6ccccc6c45)c3oc21]

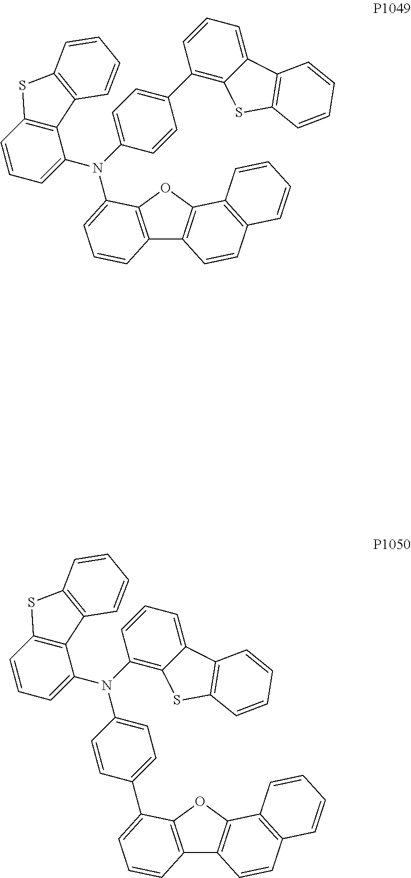 [SMILES: c1ccc2c(c1)ccc1c3cccc(N(c4ccc(-c5cccc6c5sc5ccccc56)cc4)c4cccc5sc6ccccc6c45)c3oc21 ; c1ccc2c(c1)ccc1c3cccc(-c4ccc(N(c5cccc6c5sc5ccccc56)c5cccc6sc7ccccc7c56)cc4)c3oc21]

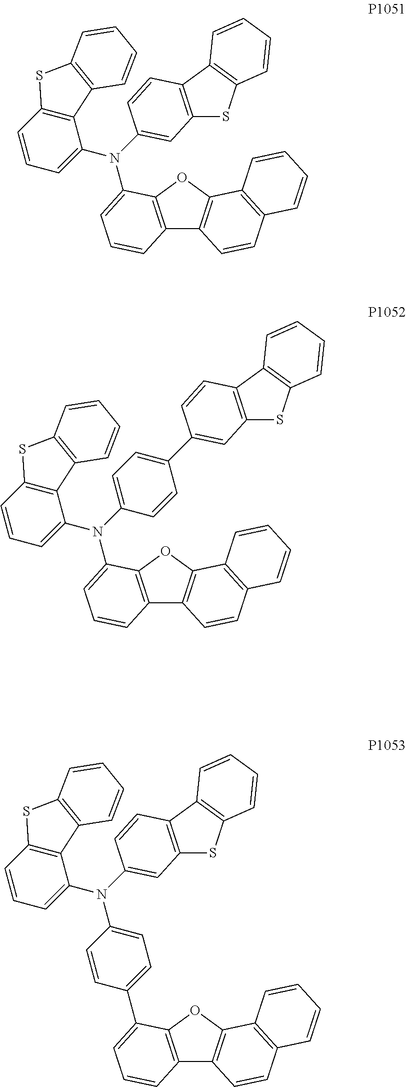 [SMILES: c1ccc2c(c1)ccc1c3cccc(N(c4ccc5c(c4)sc4ccccc45)c4cccc5sc6ccccc6c45)c3oc21 ; c1ccc2c(c1)ccc1c3cccc(N(c4ccc(-c5ccc6c(c5)sc5ccccc56)cc4)c4cccc5sc6ccccc6c45)c3oc21 ; c1ccc2c(c1)ccc1c3cccc(-c4ccc(N(c5ccc6c(c5)sc5ccccc56)c5cccc6sc7ccccc7c56)cc4)c3oc21]

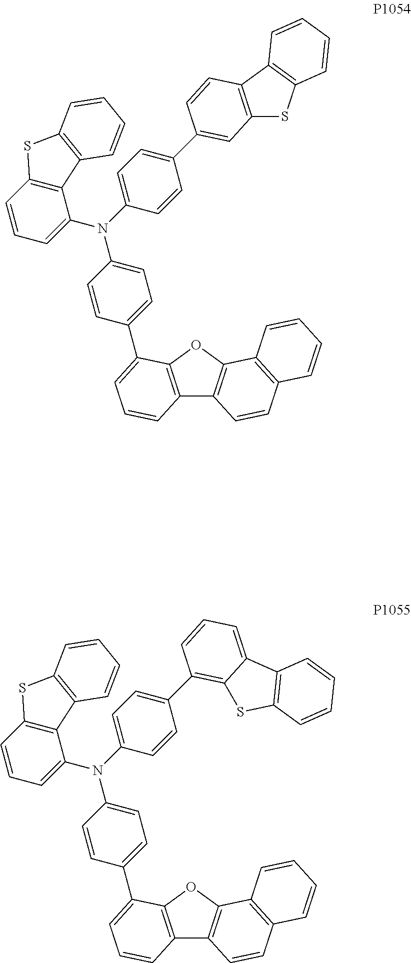 [SMILES: c1ccc2c(c1)ccc1c3cccc(-c4ccc(N(c5ccc(-c6ccc7c(c6)sc6ccccc67)cc5)c5cccc6sc7ccccc7c56)cc4)c3oc21 ; c1ccc2c(c1)ccc1c3cccc(-c4ccc(N(c5ccc(-c6cccc7c6sc6ccccc67)cc5)c5cccc6sc7ccccc7c56)cc4)c3oc21]

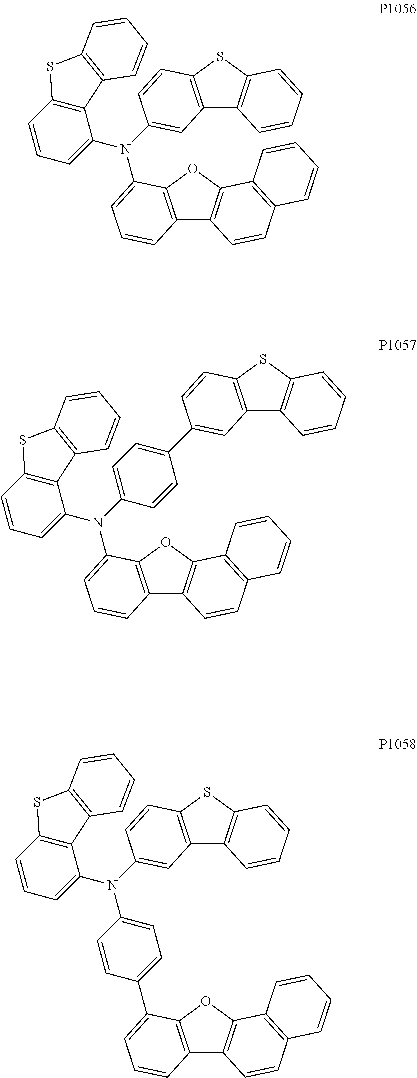 [SMILES: c1ccc2c(c1)ccc1c3cccc(N(c4ccc5sc6ccccc6c5c4)c4cccc5sc6ccccc6c45)c3oc21 ; c1ccc2c(c1)ccc1c3cccc(N(c4ccc(-c5ccc6sc7ccccc7c6c5)cc4)c4cccc5sc6ccccc6c45)c3oc21 ; c1ccc2c(c1)ccc1c3cccc(-c4ccc(N(c5ccc6sc7ccccc7c6c5)c5cccc6sc7ccccc7c56)cc4)c3oc21]

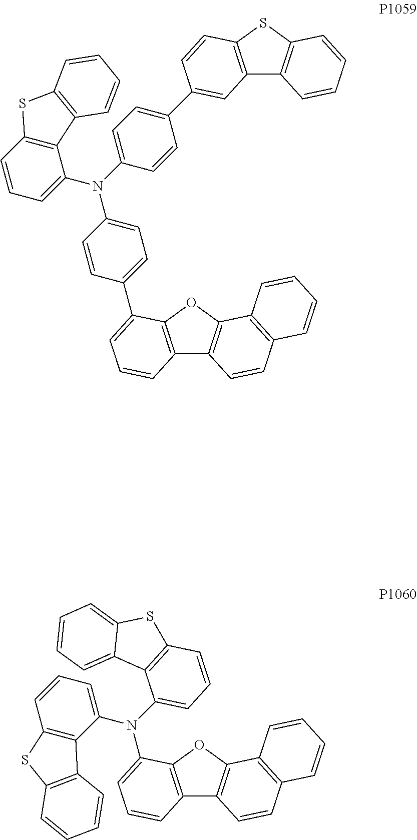 [SMILES: c1ccc2c(c1)ccc1c3cccc(-c4ccc(N(c5ccc(-c6ccc7sc8ccccc8c7c6)cc5)c5cccc6sc7ccccc7c56)cc4)c3oc21 ; c1ccc2c(c1)ccc1c3cccc(N(c4cccc5sc6ccccc6c45)c4cccc5sc6ccccc6c45)c3oc21]

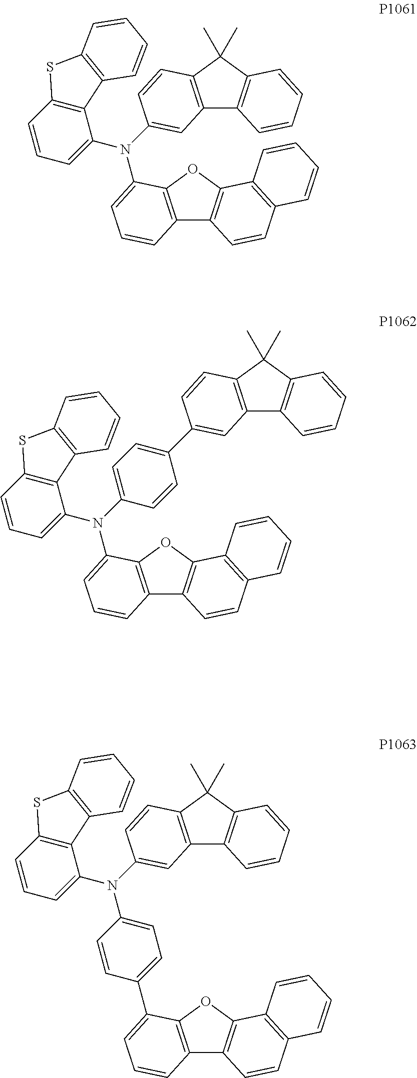 [SMILES: CC1(C)c2ccccc2-c2cc(N(c3cccc4c3oc3c5ccccc5ccc43)c3cccc4sc5ccccc5c34)ccc21 ; CC1(C)c2ccccc2-c2cc(-c3ccc(N(c4cccc5c4oc4c6ccccc6ccc54)c4cccc5sc6ccccc6c45)cc3)ccc21 ; CC1(C)c2ccccc2-c2cc(N(c3ccc(-c4cccc5c4oc4c6ccccc6ccc54)cc3)c3cccc4sc5ccccc5c34)ccc21]

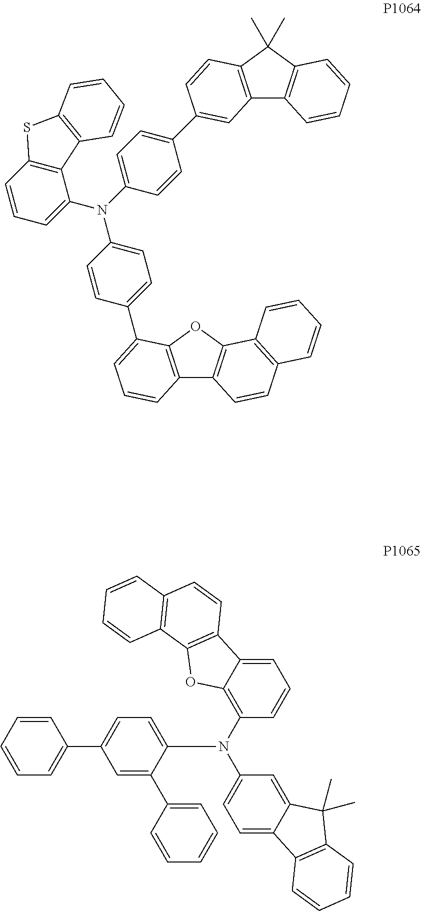 [SMILES: CC1(C)c2ccccc2-c2cc(-c3ccc(N(c4ccc(-c5cccc6c5oc5c7ccccc7ccc65)cc4)c4cccc5sc6ccccc6c45)cc3)ccc21 ; CC1(C)c2ccccc2-c2ccc(N(c3ccc(-c4ccccc4)cc3-c3ccccc3)c3cccc4c3oc3c5ccccc5ccc43)cc21]

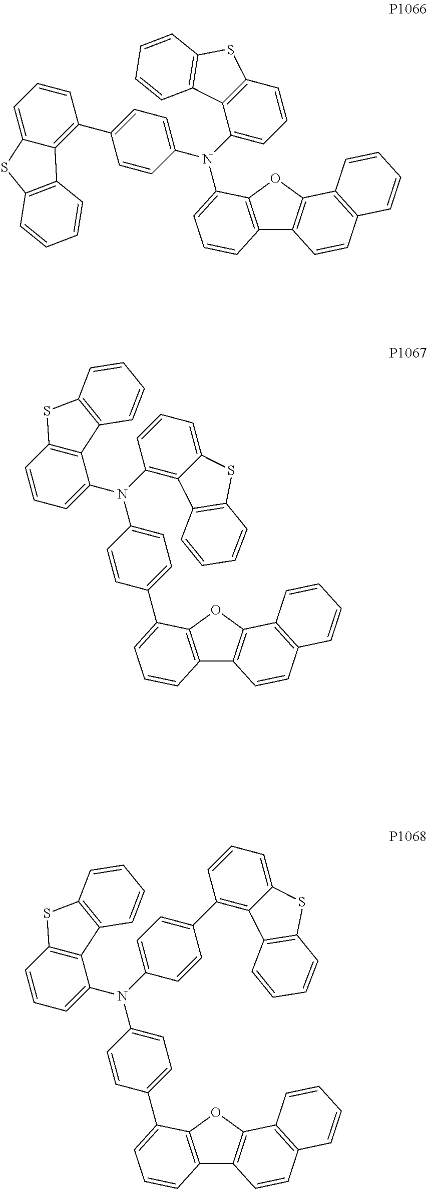 [SMILES: c1ccc2c(c1)ccc1c3cccc(N(c4ccc(-c5cccc6sc7ccccc7c56)cc4)c4cccc5sc6ccccc6c45)c3oc21 ; c1ccc2c(c1)ccc1c3cccc(-c4ccc(N(c5cccc6sc7ccccc7c56)c5cccc6sc7ccccc7c56)cc4)c3oc21 ; c1ccc2c(c1)ccc1c3cccc(-c4ccc(N(c5ccc(-c6cccc7sc8ccccc8c67)cc5)c5cccc6sc7ccccc7c56)cc4)c3oc21]

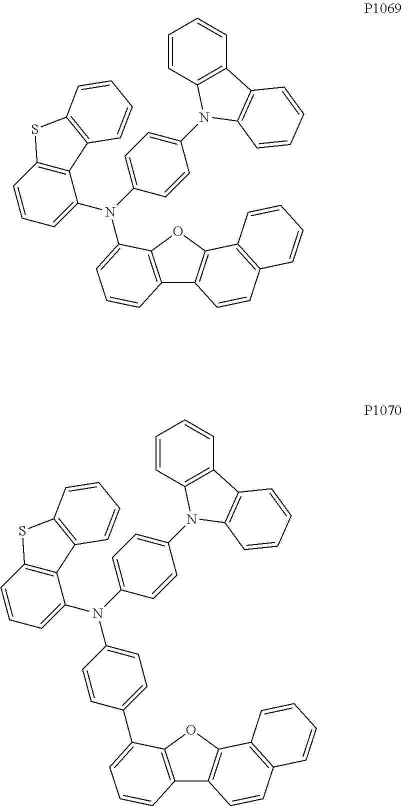 [SMILES: c1ccc2c(c1)ccc1c3cccc(N(c4ccc(-n5c6ccccc6c6ccccc65)cc4)c4cccc5sc6ccccc6c45)c3oc21 ; c1ccc2c(c1)ccc1c3cccc(-c4ccc(N(c5ccc(-n6c7ccccc7c7ccccc76)cc5)c5cccc6sc7ccccc7c56)cc4)c3oc21]

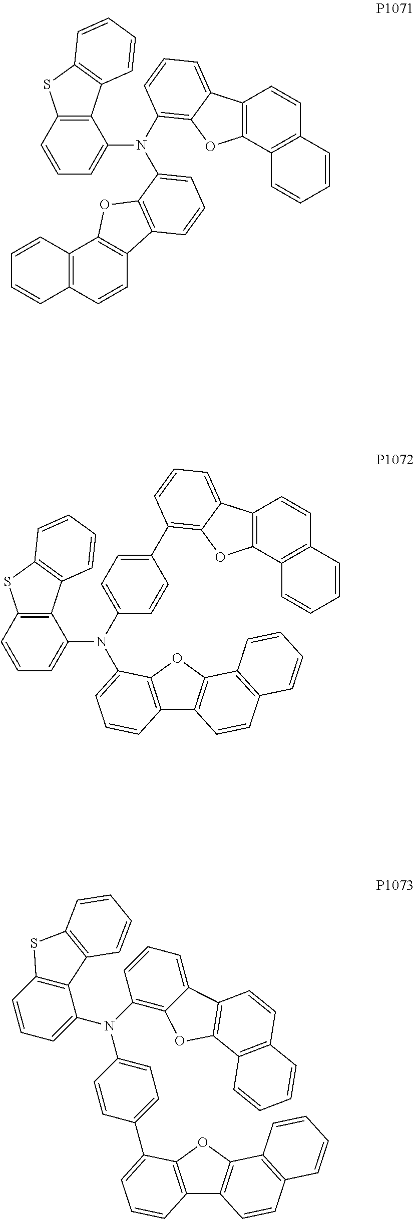 [SMILES: c1ccc2c(c1)ccc1c3cccc(N(c4cccc5c4oc4c6ccccc6ccc54)c4cccc5sc6ccccc6c45)c3oc21 ; c1ccc2c(c1)ccc1c3cccc(-c4ccc(N(c5cccc6c5oc5c7ccccc7ccc65)c5cccc6sc7ccccc7c56)cc4)c3oc21 ; c1ccc2c(c1)ccc1c3cccc(-c4ccc(N(c5cccc6c5oc5c7ccccc7ccc65)c5cccc6sc7ccccc7c56)cc4)c3oc21]

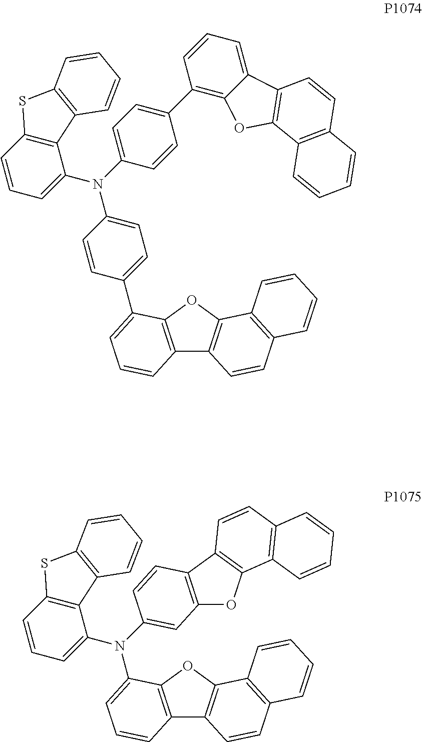 [SMILES: c1ccc2c(c1)ccc1c3cccc(-c4ccc(N(c5ccc(-c6cccc7c6oc6c8ccccc8ccc76)cc5)c5cccc6sc7ccccc7c56)cc4)c3oc21 ; c1ccc2c(c1)ccc1c3ccc(N(c4cccc5c4oc4c6ccccc6ccc54)c4cccc5sc6ccccc6c45)cc3oc21]

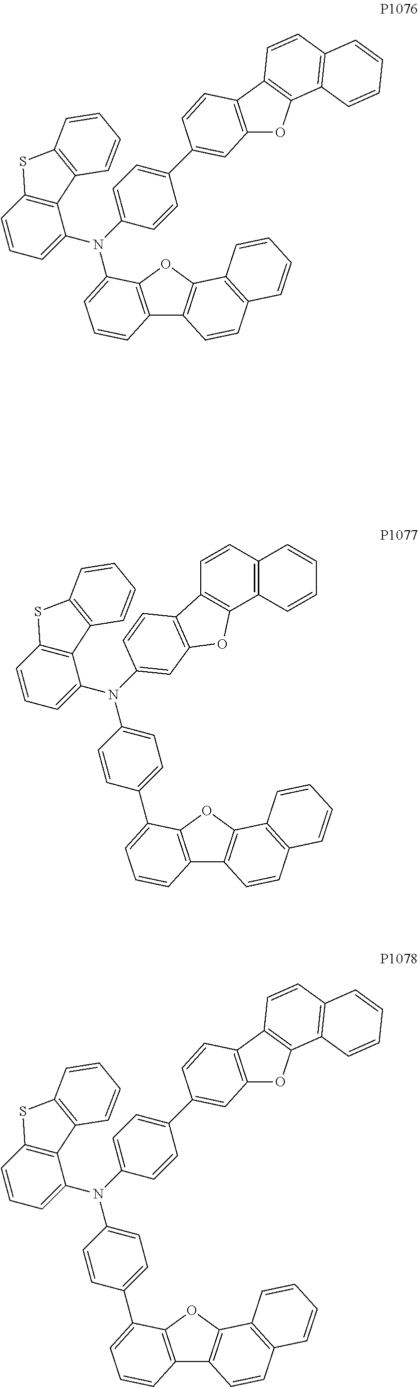 [SMILES: c1ccc2c(c1)ccc1c3ccc(-c4ccc(N(c5cccc6c5oc5c7ccccc7ccc65)c5cccc6sc7ccccc7c56)cc4)cc3oc21 ; c1ccc2c(c1)ccc1c3ccc(N(c4ccc(-c5cccc6c5oc5c7ccccc7ccc65)cc4)c4cccc5sc6ccccc6c45)cc3oc21 ; c1ccc2c(c1)ccc1c3ccc(-c4ccc(N(c5ccc(-c6cccc7c6oc6c8ccccc8ccc76)cc5)c5cccc6sc7ccccc7c56)cc4)cc3oc21]

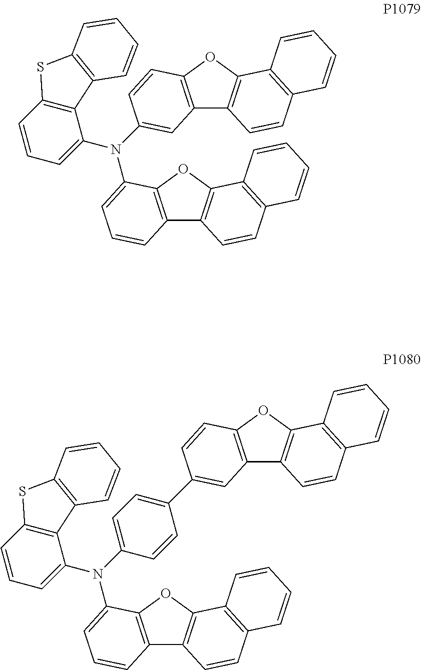 [SMILES: c1ccc2c(c1)ccc1c3cc(N(c4cccc5c4oc4c6ccccc6ccc54)c4cccc5sc6ccccc6c45)ccc3oc21 ; c1ccc2c(c1)ccc1c3cc(-c4ccc(N(c5cccc6c5oc5c7ccccc7ccc65)c5cccc6sc7ccccc7c56)cc4)ccc3oc21]

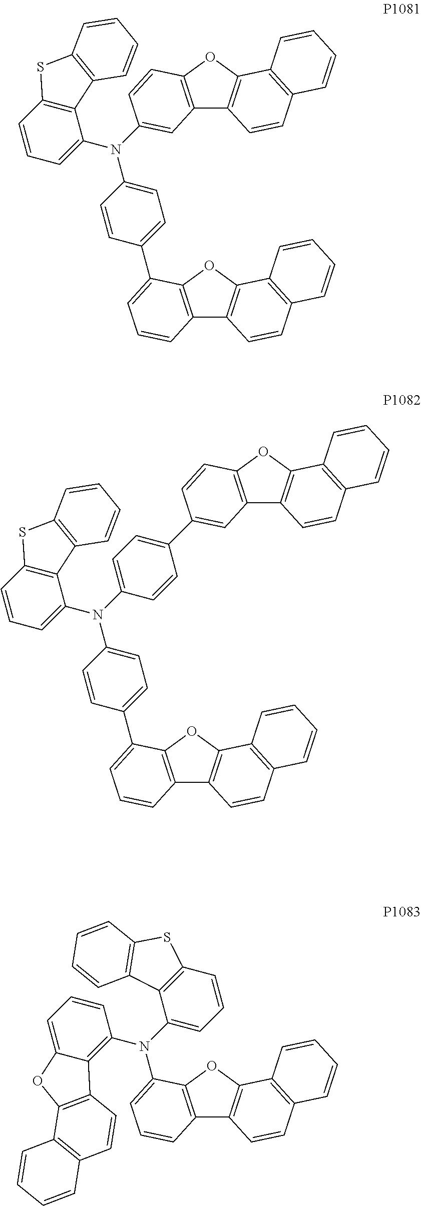 [SMILES: c1ccc2c(c1)ccc1c3cc(N(c4ccc(-c5cccc6c5oc5c7ccccc7ccc65)cc4)c4cccc5sc6ccccc6c45)ccc3oc21 ; c1ccc2c(c1)ccc1c3cc(-c4ccc(N(c5ccc(-c6cccc7c6oc6c8ccccc8ccc76)cc5)c5cccc6sc7ccccc7c56)cc4)ccc3oc21 ; c1ccc2c(c1)ccc1c3cccc(N(c4cccc5oc6c7ccccc7ccc6c45)c4cccc5sc6ccccc6c45)c3oc21]

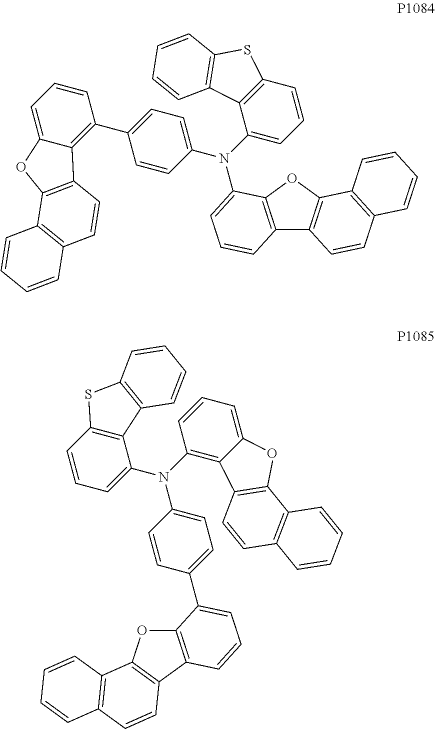 [SMILES: c1ccc2c(c1)ccc1c3cccc(N(c4ccc(-c5cccc6oc7c8ccccc8ccc7c56)cc4)c4cccc5sc6ccccc6c45)c3oc21 ; c1ccc2c(c1)ccc1c3cccc(-c4ccc(N(c5cccc6oc7c8ccccc8ccc7c56)c5cccc6sc7ccccc7c56)cc4)c3oc21]

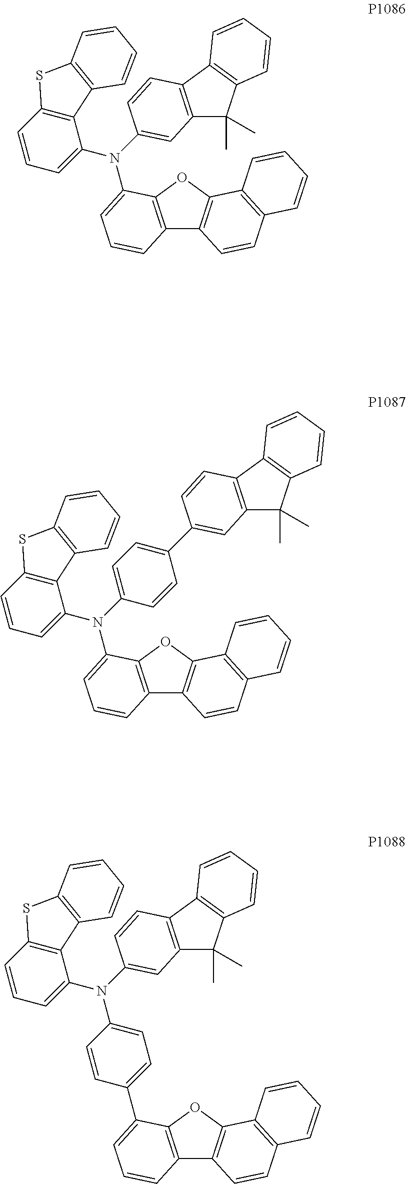 [SMILES: CC1(C)c2ccccc2-c2ccc(N(c3cccc4c3oc3c5ccccc5ccc43)c3cccc4sc5ccccc5c34)cc21 ; CC1(C)c2ccccc2-c2ccc(-c3ccc(N(c4cccc5c4oc4c6ccccc6ccc54)c4cccc5sc6ccccc6c45)cc3)cc21 ; CC1(C)c2ccccc2-c2ccc(N(c3ccc(-c4cccc5c4oc4c6ccccc6ccc54)cc3)c3cccc4sc5ccccc5c34)cc21]

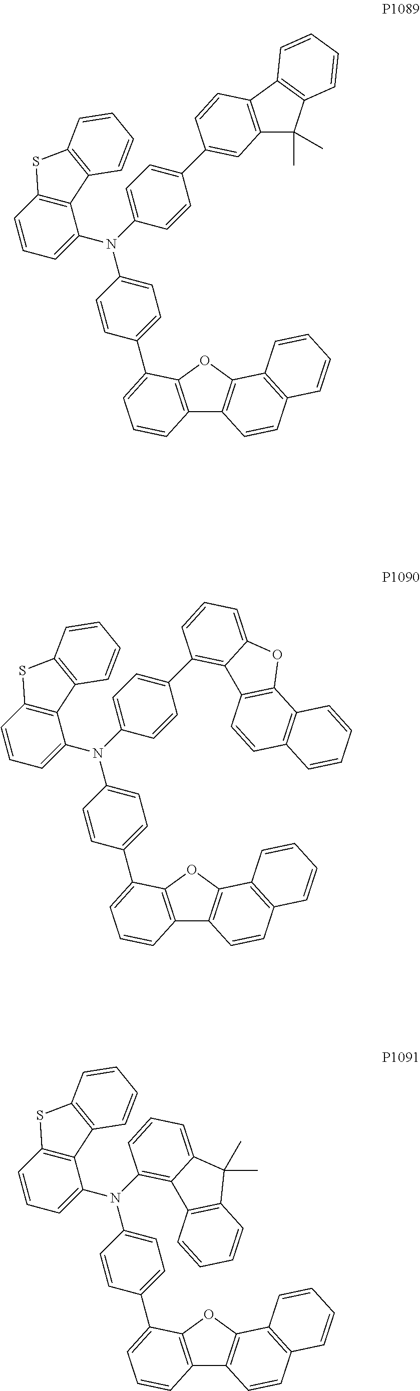 [SMILES: CC1(C)c2ccccc2-c2ccc(-c3ccc(N(c4ccc(-c5cccc6c5oc5c7ccccc7ccc65)cc4)c4cccc5sc6ccccc6c45)cc3)cc21 ; c1ccc2c(c1)ccc1c3cccc(-c4ccc(N(c5ccc(-c6cccc7oc8c9ccccc9ccc8c67)cc5)c5cccc6sc7ccccc7c56)cc4)c3oc21 ; CC1(C)c2ccccc2-c2c(N(c3ccc(-c4cccc5c4oc4c6ccccc6ccc54)cc3)c3cccc4sc5ccccc5c34)cccc21]

According to one embodiment of the present invention, any one of the hole transport layer or the hole transport auxiliary layer of the organic light emitting diode may be made of an organic compound represented by Chemical Formula 1, and in this case, the other not made of the organic compound represented by Chemical Formula 1 among the hole transport layer and the hole transport auxiliary layer may be made of a hole transport material used in the art. Specific hole transport materials may include a compound selected from the group consisting of TPD, NPD, CBP, N4,N4,N4′,N4′-tetra([1,1′-biphenyl]-4-yl)-[1,1′-biphenyl]-4,4′-diamine, N-(biphenyl-4-yl)-9,9-dimethyl-N-(4-(9-phenyl-9H-carbazol-3-yl)phenyl)-9H-fluorene-2-amine, N-(biphenyl-4-yl)-N-(4-(9-phenyl-9H-carbazol-3-yl)phenyl)biphenyl)-4-amine and the like, but is not limited thereto.

According to the present invention, the light emitting layer may be formed by being doped with a dopant to increase the luminous efficiency and the like of the host and the device, and the light emitting layer may emit blue, green, or red light, but is not limited thereto, and may be combined with light emitting layers of various colors and color coordinates used in the organic light emitting diode. For example, CIEx coordinates of the blue light emitting layer may range from 0.01 to 0.15 and CIEy coordinates thereof may range from 0.03 to 0.07, while CIEx coordinates of the green light emitting layer may range from 0.19 to 0.32 and CIEy coordinates thereof may range from 0.65 to 0.76.

A dopant material and a host material may be selected depending on a color selected for the light emitting layer included in the organic light emitting diode of the present invention. For example, a doping concentration of the dopant may be adjusted in the range of 1 to 20 wt % based on the total weight of the host, but is not limited thereto, and may be, for example, 3 to 15 wt %, for example, 5 to 10 wt %, for example, 3 to 8 wt %, and for example, 2 to 7 wt %, but is not limited thereto.

For example, the host of the light emitting layer is a common material used in the art and may include 9,10-Bis(2-naphthyl)anthracene(ADN), CBP(4,4′-N,N′-dicarbazole-biphenyl), and mCP(1,3-bis(carbazol-9-yl), and the like, but is not limited thereto.

For example, the dopant of the light emitting layer is a common material used in the art and may include N1,N1,N6,N6-tetrakis(4-(1-silyl)phenyl)pyrene-1,6-diamine, iridium complex metal compound (e.g. Ir(ppy)₃), and the like, but is not limited thereto.

An electron transport layer and an electron injection layer may be sequentially stacked between the light emitting layer and the second electrode. The material of the electron transport layer requires high electron mobility, and electrons may be stably supplied to the light emitting layer through smooth electron transport.

For example, the material of the electron transport layer may include Alq₃(tris(8-hydroxyquinolinato)aluminum), Liq(8-hydroxyquinolinolatolithium), PBD(2-(4-biphenylyl)-5-(4-tert-butylphenyl)-1,3,4oxadiazole), TAZ(3-(4-biphenyl)4-phenyl-5-tert-butylphenyl-1,2,4-triazole), BAlq(bis(2-methyl-8-quinolinolate)-4-(phenylphenolato)aluminium), TPBi(2,2′,2-(1,3,5-benzinetriyl)-tris(1-phenyl-1-H-benzimidazole), ZADN(2-[4-(9,10-Di-2-naphthalen2-yl-2-anthracen-2-yl)phenyl]-1-phenyl-1H-benzoimidazole), and the like, and may be used in combination with a metal compound. Examples of the metal compounds may include Liq, LiF, NaF, KF, RbF, CsF, FrF, BeF₂, MgF₂, CaF₂, SrF₂, BaF₂, RaF₂, and the like, but are not limited thereto.

The electron injection layer serves to smoothly inject electrons, the material of the electron injection layer may be made of a metal compound, and the metal compound may include any one or more among Liq, LiF, NaF, KF, RbF, CsF, FrF, BeF₂, MgF₂, CaF₂, SrF₂, BaF₂, and RaF₂, for example, but is not limited thereto.

### SYNTHESIS EXAMPLE

### I. Synthesis Example of Intermediate A (or Referred to as “Sub A”)

The intermediate A (Sub A) may be synthesized according to Reaction Formula 1 below, but is not necessarily limited thereto. (In Reaction Formula 1 below, X═O or S, Hal=Br or Cl)

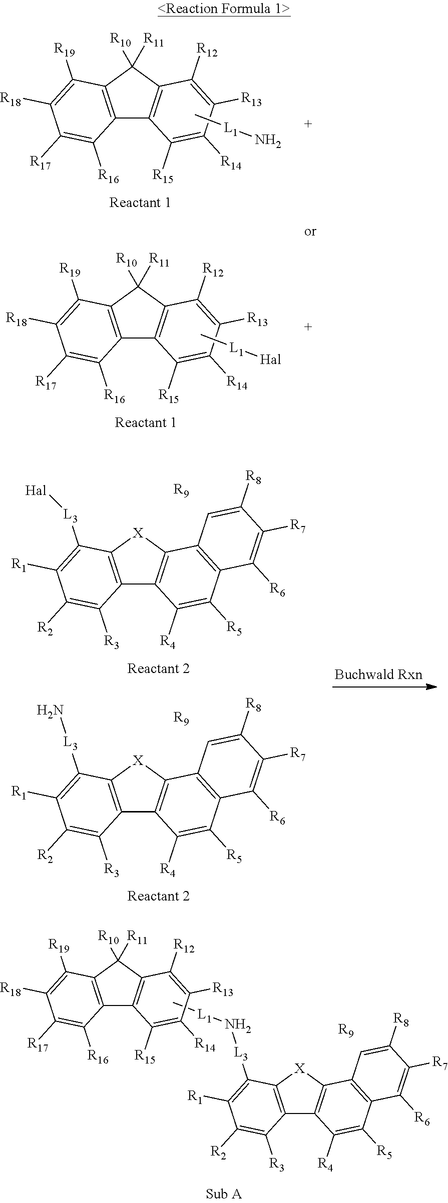

The definition of L₁, L₃, and R₁ to R₁₉ of Sub A are the same as those defined in Chemical Formula 1.

The compound belonging to Sub A may be any one of Products Sub 1 to Sub 120 below, but it is only an example and not limited.

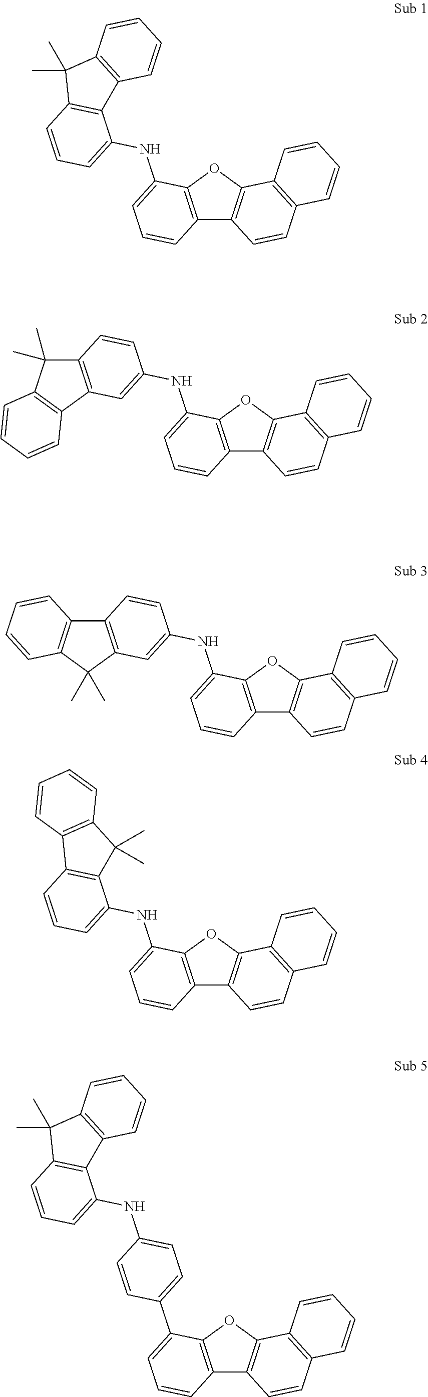 [SMILES: CC1(C)c2ccccc2-c2c(Nc3cccc4c3oc3c5ccccc5ccc43)cccc21 ; CC1(C)c2ccccc2-c2cc(Nc3cccc4c3oc3c5ccccc5ccc43)ccc21 ; CC1(C)c2ccccc2-c2ccc(Nc3cccc4c3oc3c5ccccc5ccc43)cc21 ; CC1(C)c2ccccc2-c2cccc(Nc3cccc4c3oc3c5ccccc5ccc43)c21 ; CC1(C)c2ccccc2-c2c(Nc3ccc(-c4cccc5c4oc4c6ccccc6ccc54)cc3)cccc21]

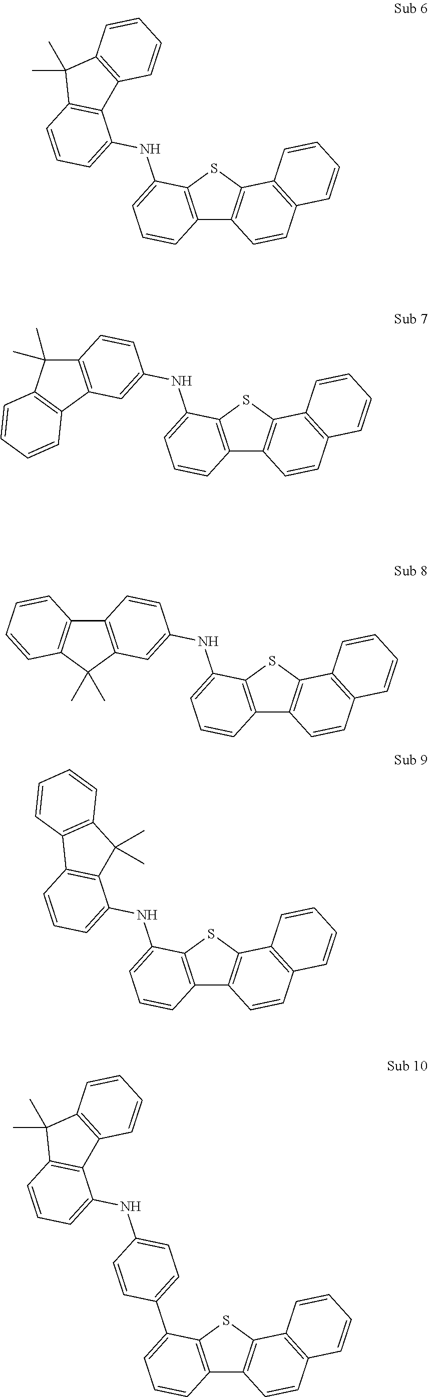 [SMILES: CC1(C)c2ccccc2-c2c(Nc3cccc4c3sc3c5ccccc5ccc43)cccc21 ; CC1(C)c2ccccc2-c2cc(Nc3cccc4c3sc3c5ccccc5ccc43)ccc21 ; CC1(C)c2ccccc2-c2ccc(Nc3cccc4c3sc3c5ccccc5ccc43)cc21 ; CC1(C)c2ccccc2-c2cccc(Nc3cccc4c3sc3c5ccccc5ccc43)c21 ; CC1(C)c2ccccc2-c2c(Nc3ccc(-c4cccc5c4sc4c6ccccc6ccc54)cc3)cccc21]

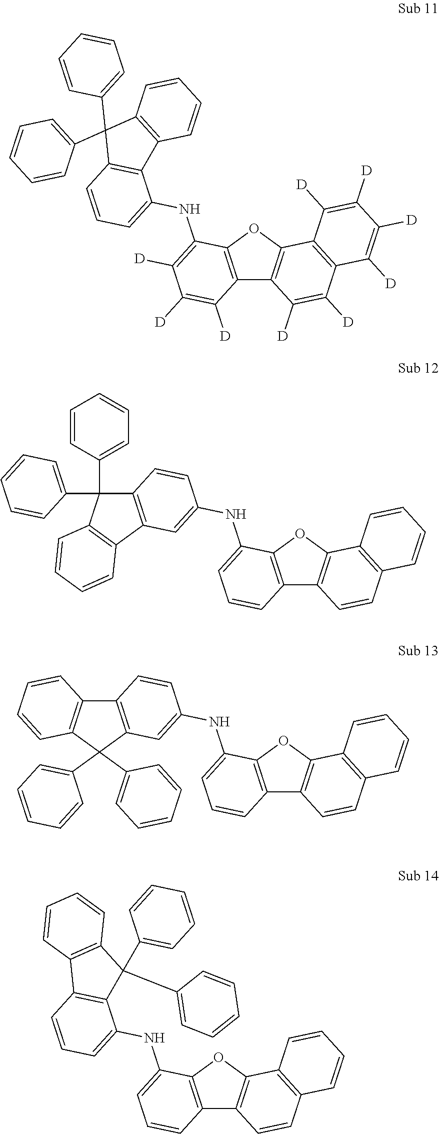 [SMILES: [2H]c1c([2H])c([2H])c2c(oc3c4c([2H])c([2H])c([2H])c([2H])c4c([2H])c([2H])c32)c1Nc1cccc2c1-c1ccccc1C2(c1ccccc1)c1ccccc1 ; c1ccc(C2(c3ccccc3)c3ccccc3-c3cc(Nc4cccc5c4oc4c6ccccc6ccc54)ccc32)cc1 ; c1ccc(C2(c3ccccc3)c3ccccc3-c3ccc(Nc4cccc5c4oc4c6ccccc6ccc54)cc32)cc1 ; c1ccc(C2(c3ccccc3)c3ccccc3-c3cccc(Nc4cccc5c4oc4c6ccccc6ccc54)c32)cc1]

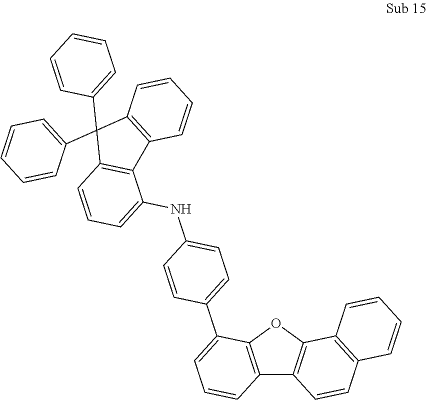 [SMILES: c1ccc(C2(c3ccccc3)c3ccccc3-c3c(Nc4ccc(-c5cccc6c5oc5c7ccccc7ccc65)cc4)cccc32)cc1]

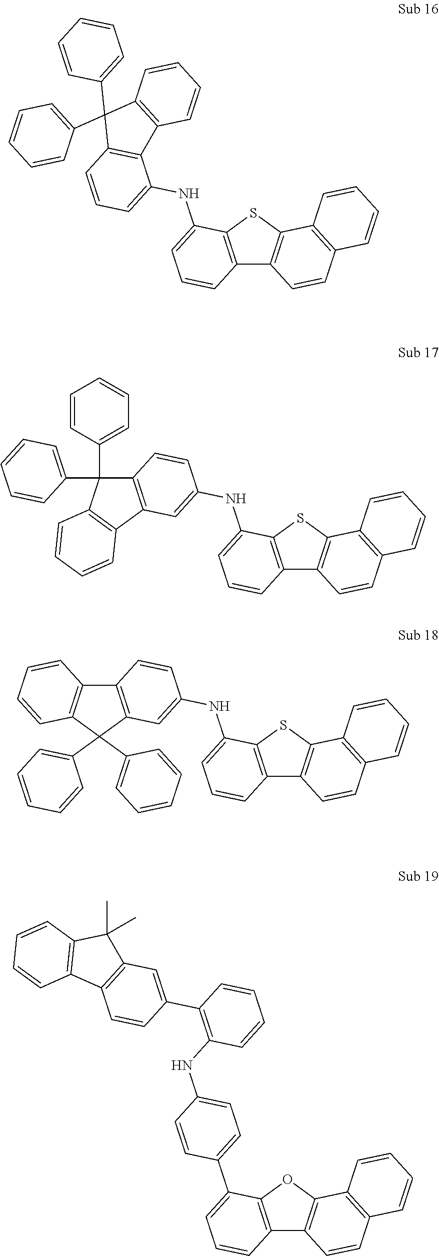 [SMILES: c1ccc(C2(c3ccccc3)c3ccccc3-c3c(Nc4cccc5c4sc4c6ccccc6ccc54)cccc32)cc1 ; c1ccc(C2(c3ccccc3)c3ccccc3-c3cc(Nc4cccc5c4sc4c6ccccc6ccc54)ccc32)cc1 ; c1ccc(C2(c3ccccc3)c3ccccc3-c3ccc(Nc4cccc5c4sc4c6ccccc6ccc54)cc32)cc1 ; CC1(C)c2ccccc2-c2ccc(-c3ccccc3Nc3ccc(-c4cccc5c4oc4c6ccccc6ccc54)cc3)cc21]

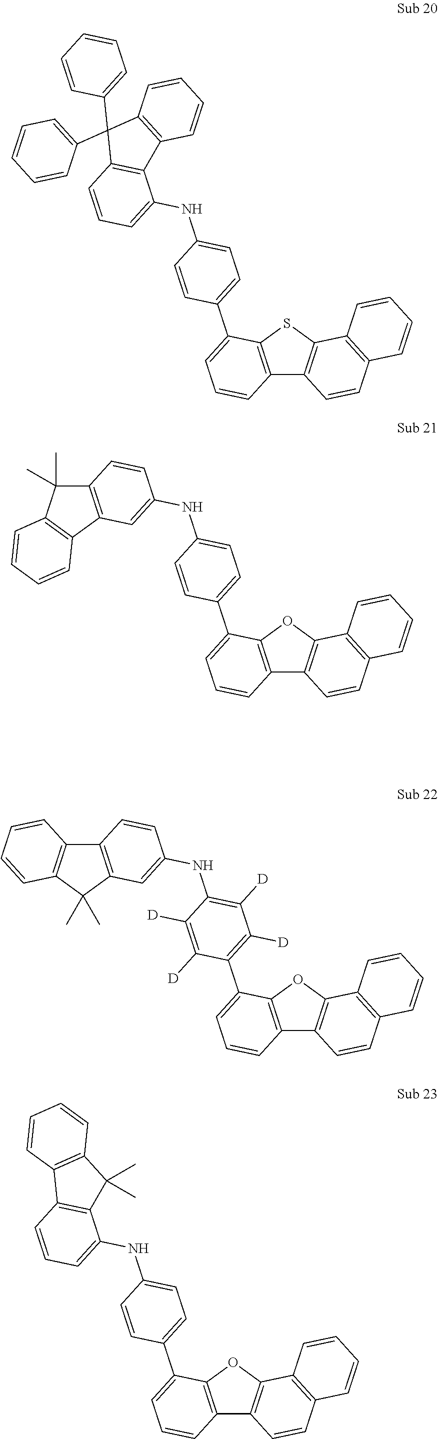 [SMILES: c1ccc(C2(c3ccccc3)c3ccccc3-c3c(Nc4ccc(-c5cccc6c5sc5c7ccccc7ccc65)cc4)cccc32)cc1 ; CC1(C)c2ccccc2-c2cc(Nc3ccc(-c4cccc5c4oc4c6ccccc6ccc54)cc3)ccc21 ; [2H]c1c([2H])c(-c2cccc3c2oc2c4ccccc4ccc32)c([2H])c([2H])c1Nc1ccc2c(c1)C(C)(C)c1ccccc1-2 ; CC1(C)c2ccccc2-c2cccc(Nc3ccc(-c4cccc5c4oc4c6ccccc6ccc54)cc3)c21]

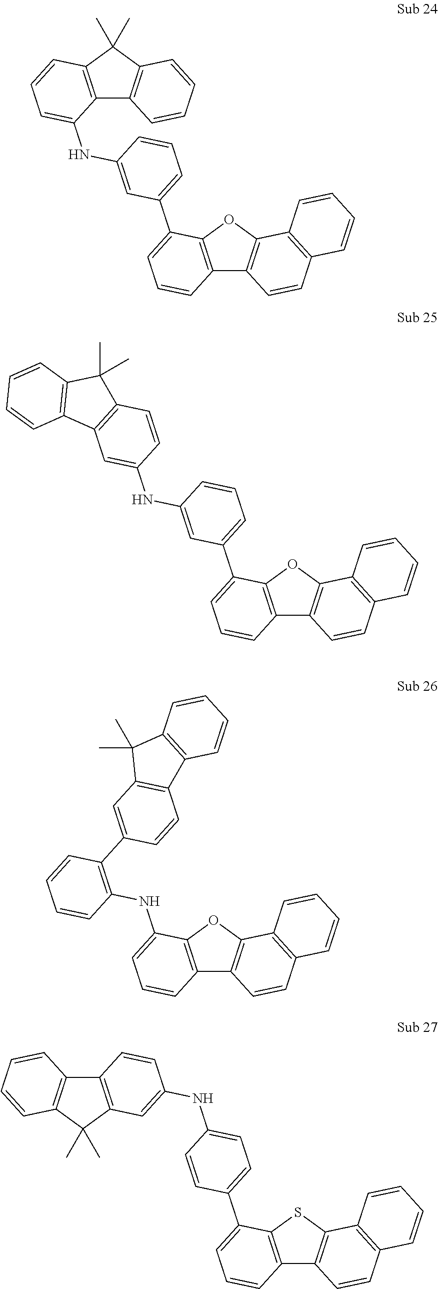 [SMILES: CC1(C)c2ccccc2-c2c(Nc3cccc(-c4cccc5c4oc4c6ccccc6ccc54)c3)cccc21 ; CC1(C)c2ccccc2-c2cc(Nc3cccc(-c4cccc5c4oc4c6ccccc6ccc54)c3)ccc21 ; CC1(C)c2ccccc2-c2ccc(-c3ccccc3Nc3cccc4c3oc3c5ccccc5ccc43)cc21 ; CC1(C)c2ccccc2-c2ccc(Nc3ccc(-c4cccc5c4sc4c6ccccc6ccc54)cc3)cc21]

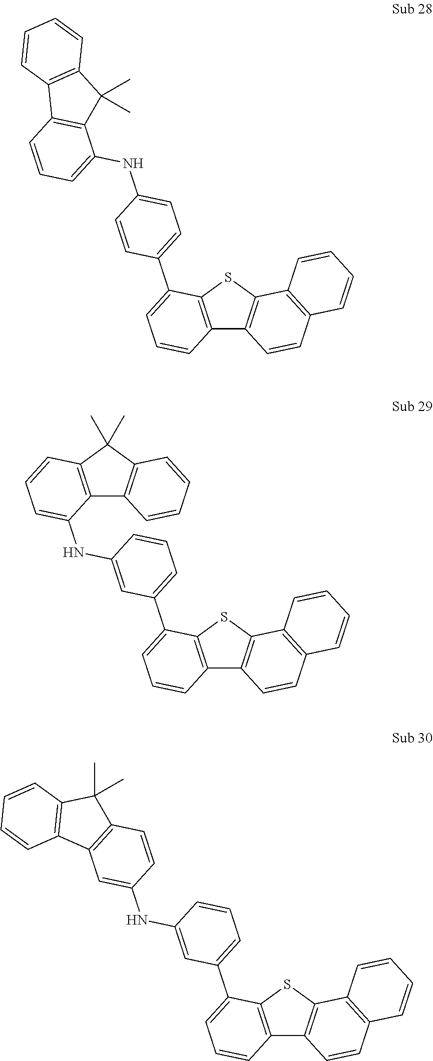 [SMILES: CC1(C)c2ccccc2-c2cccc(Nc3ccc(-c4cccc5c4sc4c6ccccc6ccc54)cc3)c21 ; CC1(C)c2ccccc2-c2c(Nc3cccc(-c4cccc5c4sc4c6ccccc6ccc54)c3)cccc21 ; CC1(C)c2ccccc2-c2cc(Nc3cccc(-c4cccc5c4sc4c6ccccc6ccc54)c3)ccc21]

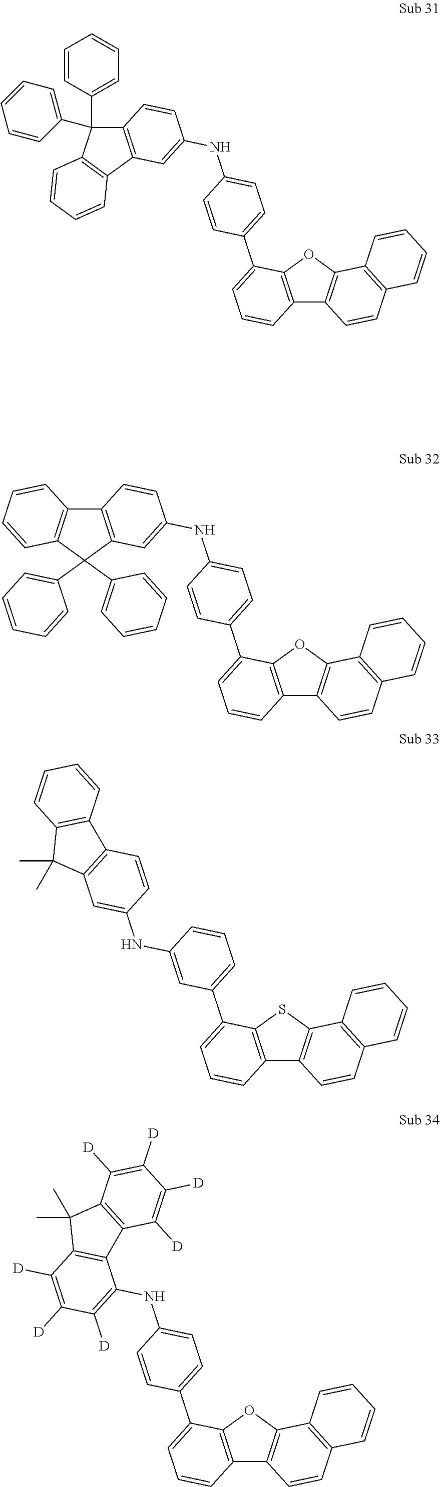 [SMILES: c1ccc(C2(c3ccccc3)c3ccccc3-c3cc(Nc4ccc(-c5cccc6c5oc5c7ccccc7ccc65)cc4)ccc32)cc1 ; c1ccc(C2(c3ccccc3)c3ccccc3-c3ccc(Nc4ccc(-c5cccc6c5oc5c7ccccc7ccc65)cc4)cc32)cc1 ; CC1(C)c2ccccc2-c2ccc(Nc3cccc(-c4cccc5c4sc4c6ccccc6ccc54)c3)cc21 ; [2H]c1c([2H])c([2H])c2c(c1[2H])-c1c(Nc3ccc(-c4cccc5c4oc4c6ccccc6ccc54)cc3)c([2H])c([2H])c([2H])c1C2(C)C]

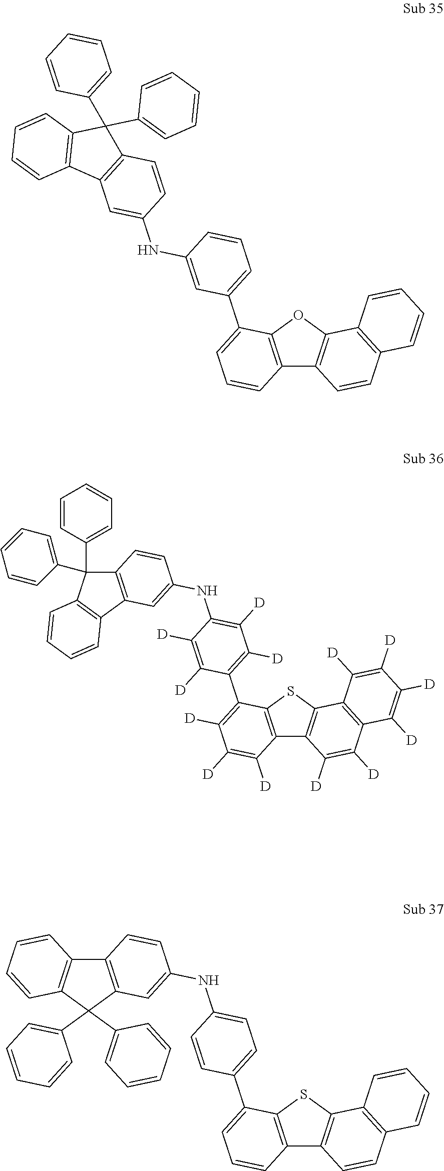 [SMILES: c1ccc(C2(c3ccccc3)c3ccccc3-c3cc(Nc4cccc(-c5cccc6c5oc5c7ccccc7ccc65)c4)ccc32)cc1 ; [2H]c1c([2H])c(-c2c([2H])c([2H])c([2H])c3c2sc2c4c([2H])c([2H])c([2H])c([2H])c4c([2H])c([2H])c23)c([2H])c([2H])c1Nc1ccc2c(c1)-c1ccccc1C2(c1ccccc1)c1ccccc1 ; c1ccc(C2(c3ccccc3)c3ccccc3-c3ccc(Nc4ccc(-c5cccc6c5sc5c7ccccc7ccc65)cc4)cc32)cc1]

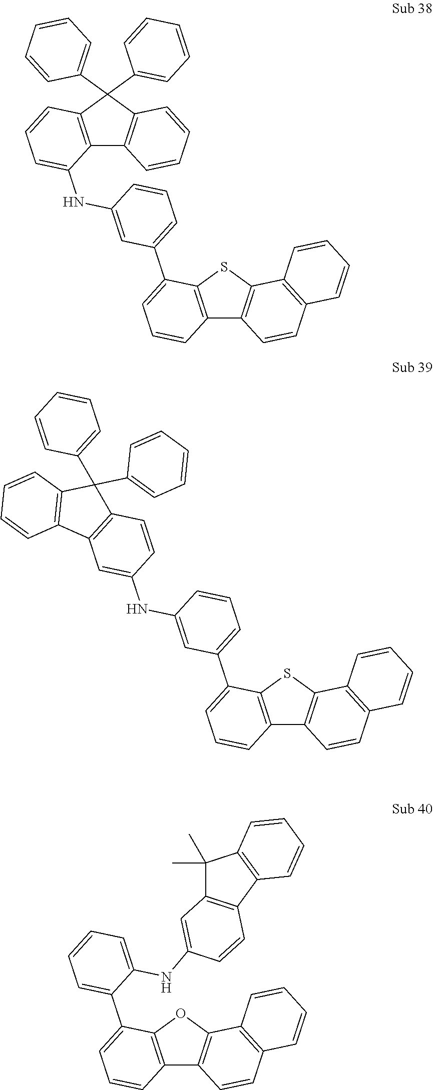 [SMILES: c1ccc(C2(c3ccccc3)c3ccccc3-c3c(Nc4cccc(-c5cccc6c5sc5c7ccccc7ccc65)c4)cccc32)cc1 ; c1ccc(C2(c3ccccc3)c3ccccc3-c3cc(Nc4cccc(-c5cccc6c5sc5c7ccccc7ccc65)c4)ccc32)cc1 ; CC1(C)c2ccccc2-c2ccc(Nc3ccccc3-c3cccc4c3oc3c5ccccc5ccc43)cc21]

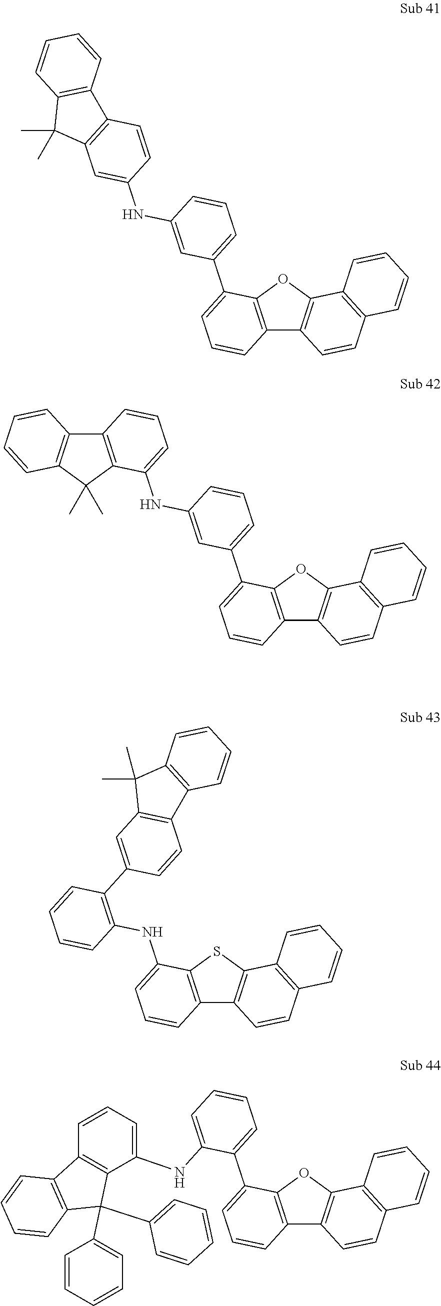 [SMILES: CC1(C)c2ccccc2-c2ccc(Nc3cccc(-c4cccc5c4oc4c6ccccc6ccc54)c3)cc21 ; CC1(C)c2ccccc2-c2cccc(Nc3cccc(-c4cccc5c4oc4c6ccccc6ccc54)c3)c21 ; CC1(C)c2ccccc2-c2ccc(-c3ccccc3Nc3cccc4c3sc3c5ccccc5ccc43)cc21 ; c1ccc(C2(c3ccccc3)c3ccccc3-c3cccc(Nc4ccccc4-c4cccc5c4oc4c6ccccc6ccc54)c32)cc1]

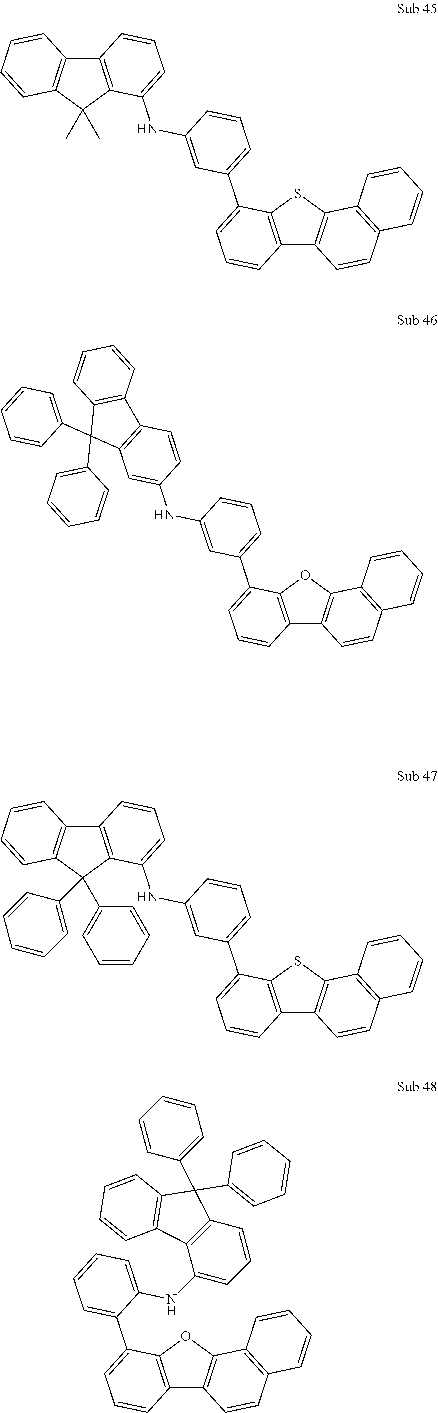 [SMILES: CC1(C)c2ccccc2-c2cccc(Nc3cccc(-c4cccc5c4sc4c6ccccc6ccc54)c3)c21 ; c1ccc(C2(c3ccccc3)c3ccccc3-c3ccc(Nc4cccc(-c5cccc6c5oc5c7ccccc7ccc65)c4)cc32)cc1 ; c1ccc(C2(c3ccccc3)c3ccccc3-c3cccc(Nc4cccc(-c5cccc6c5sc5c7ccccc7ccc65)c4)c32)cc1 ; c1ccc(C2(c3ccccc3)c3ccccc3-c3c(Nc4ccccc4-c4cccc5c4oc4c6ccccc6ccc54)cccc32)cc1]

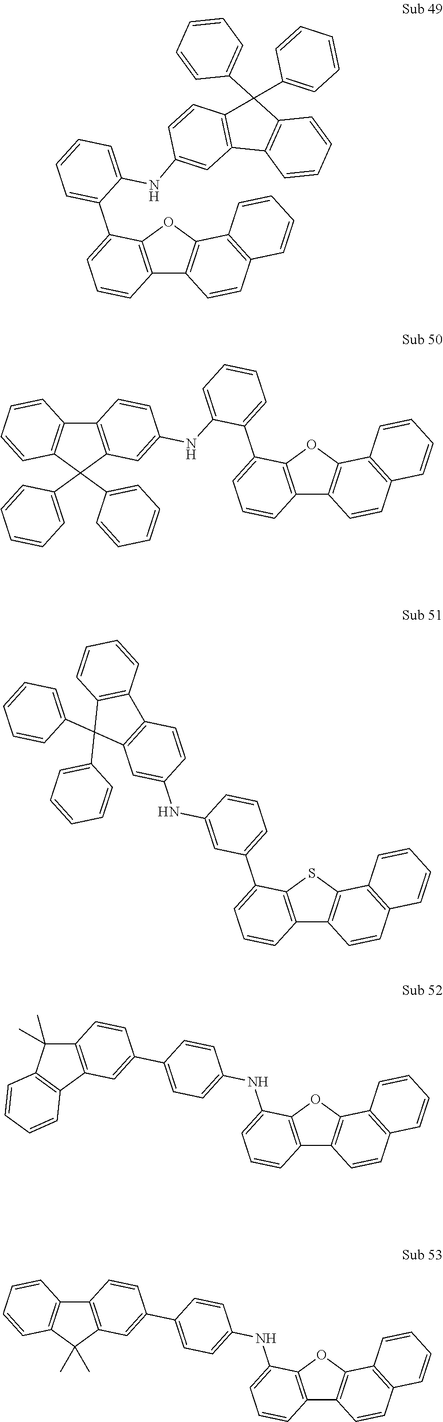 [SMILES: c1ccc(C2(c3ccccc3)c3ccccc3-c3cc(Nc4ccccc4-c4cccc5c4oc4c6ccccc6ccc54)ccc32)cc1 ; c1ccc(C2(c3ccccc3)c3ccccc3-c3ccc(Nc4ccccc4-c4cccc5c4oc4c6ccccc6ccc54)cc32)cc1 ; c1ccc(C2(c3ccccc3)c3ccccc3-c3ccc(Nc4cccc(-c5cccc6c5sc5c7ccccc7ccc65)c4)cc32)cc1 ; CC1(C)c2ccccc2-c2cc(-c3ccc(Nc4cccc5c4oc4c6ccccc6ccc54)cc3)ccc21 ; CC1(C)c2ccccc2-c2ccc(-c3ccc(Nc4cccc5c4oc4c6ccccc6ccc54)cc3)cc21]

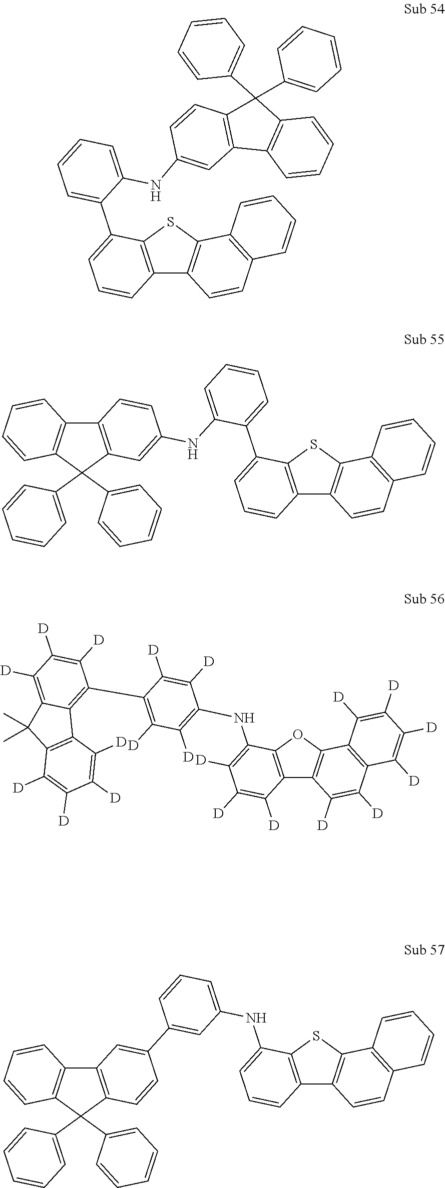 [SMILES: c1ccc(C2(c3ccccc3)c3ccccc3-c3cc(Nc4ccccc4-c4cccc5c4sc4c6ccccc6ccc54)ccc32)cc1 ; c1ccc(C2(c3ccccc3)c3ccccc3-c3ccc(Nc4ccccc4-c4cccc5c4sc4c6ccccc6ccc54)cc32)cc1 ; [2H]c1c([2H])c([2H])c2c(c1[2H])-c1c(-c3c([2H])c([2H])c(Nc4c([2H])c([2H])c([2H])c5c4oc4c6c([2H])c([2H])c([2H])c([2H])c6c([2H])c([2H])c45)c([2H])c3[2H])c([2H])c([2H])c([2H])c1C2(C)C ; c1ccc(C2(c3ccccc3)c3ccccc3-c3cc(-c4cccc(Nc5cccc6c5sc5c7ccccc7ccc65)c4)ccc32)cc1]

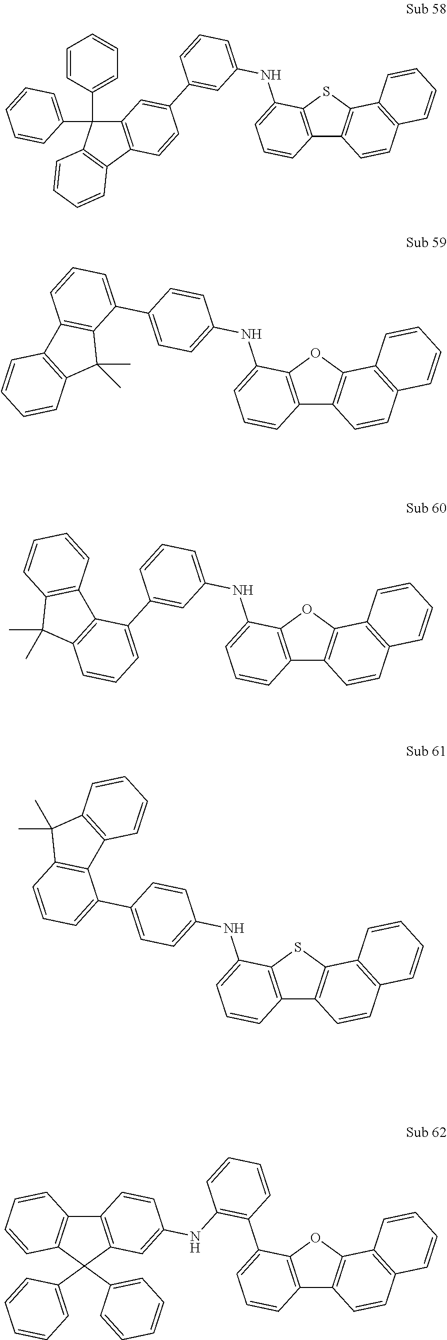 [SMILES: c1ccc(C2(c3ccccc3)c3ccccc3-c3ccc(-c4cccc(Nc5cccc6c5sc5c7ccccc7ccc65)c4)cc32)cc1 ; CC1(C)c2ccccc2-c2cccc(-c3ccc(Nc4cccc5c4oc4c6ccccc6ccc54)cc3)c21 ; CC1(C)c2ccccc2-c2c(-c3cccc(Nc4cccc5c4oc4c6ccccc6ccc54)c3)cccc21 ; CC1(C)c2ccccc2-c2c(-c3ccc(Nc4cccc5c4sc4c6ccccc6ccc54)cc3)cccc21 ; c1ccc(C2(c3ccccc3)c3ccccc3-c3ccc(Nc4ccccc4-c4cccc5c4oc4c6ccccc6ccc54)cc32)cc1]

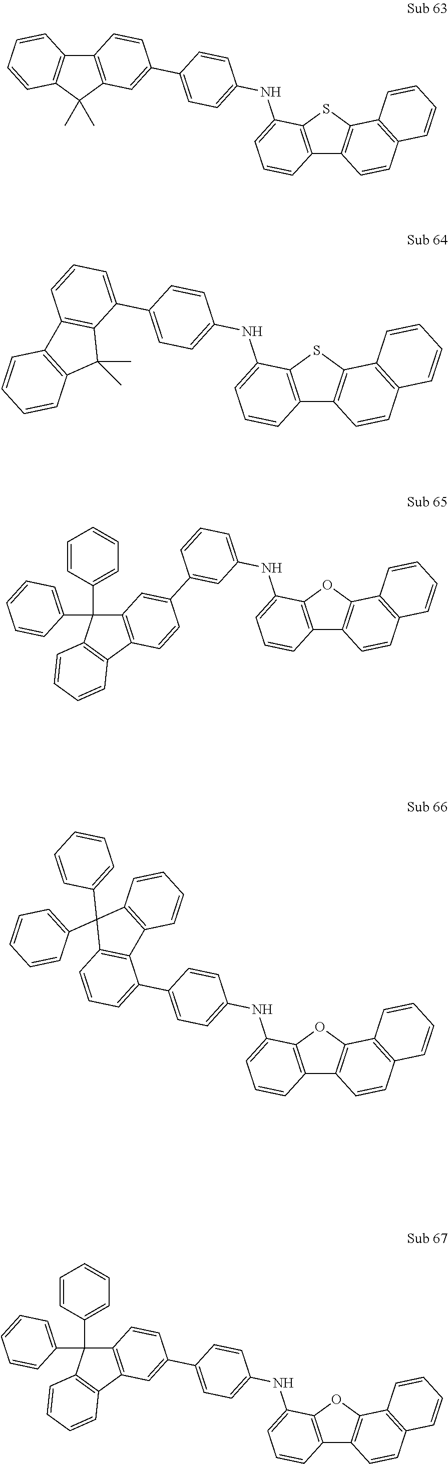 [SMILES: CC1(C)c2ccccc2-c2ccc(-c3ccc(Nc4cccc5c4sc4c6ccccc6ccc54)cc3)cc21 ; CC1(C)c2ccccc2-c2cccc(-c3ccc(Nc4cccc5c4sc4c6ccccc6ccc54)cc3)c21 ; c1ccc(C2(c3ccccc3)c3ccccc3-c3ccc(-c4cccc(Nc5cccc6c5oc5c7ccccc7ccc65)c4)cc32)cc1 ; c1ccc(C2(c3ccccc3)c3ccccc3-c3c(-c4ccc(Nc5cccc6c5oc5c7ccccc7ccc65)cc4)cccc32)cc1 ; c1ccc(C2(c3ccccc3)c3ccccc3-c3cc(-c4ccc(Nc5cccc6c5oc5c7ccccc7ccc65)cc4)ccc32)cc1]

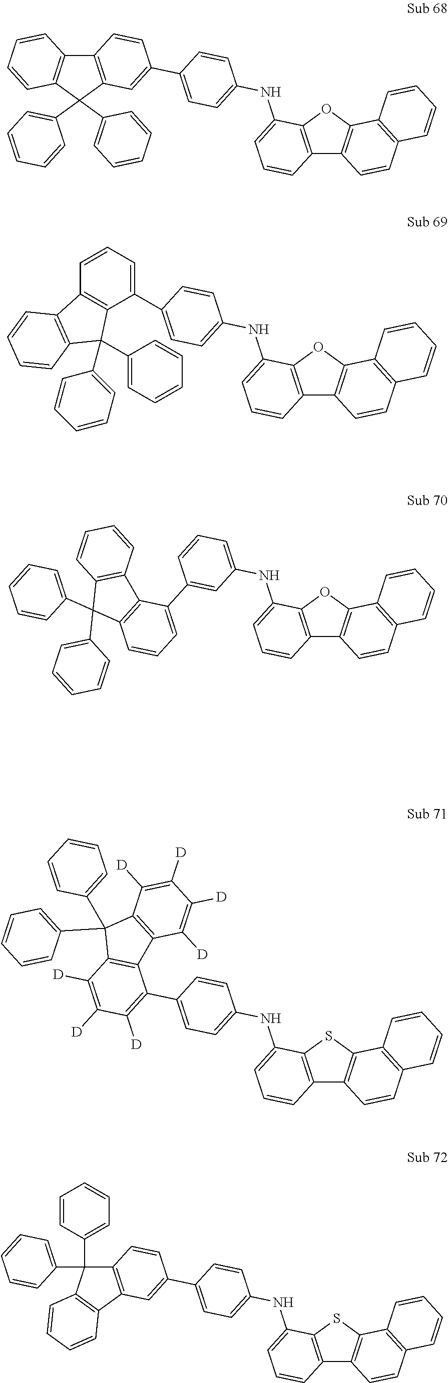 [SMILES: c1ccc(C2(c3ccccc3)c3ccccc3-c3ccc(-c4ccc(Nc5cccc6c5oc5c7ccccc7ccc65)cc4)cc32)cc1 ; c1ccc(C2(c3ccccc3)c3ccccc3-c3cccc(-c4ccc(Nc5cccc6c5oc5c7ccccc7ccc65)cc4)c32)cc1 ; c1ccc(C2(c3ccccc3)c3ccccc3-c3c(-c4cccc(Nc5cccc6c5oc5c7ccccc7ccc65)c4)cccc32)cc1 ; [2H]c1c([2H])c([2H])c2c(c1[2H])-c1c(-c3ccc(Nc4cccc5c4sc4c6ccccc6ccc54)cc3)c([2H])c([2H])c([2H])c1C2(c1ccccc1)c1ccccc1 ; c1ccc(C2(c3ccccc3)c3ccccc3-c3cc(-c4ccc(Nc5cccc6c5sc5c7ccccc7ccc65)cc4)ccc32)cc1]

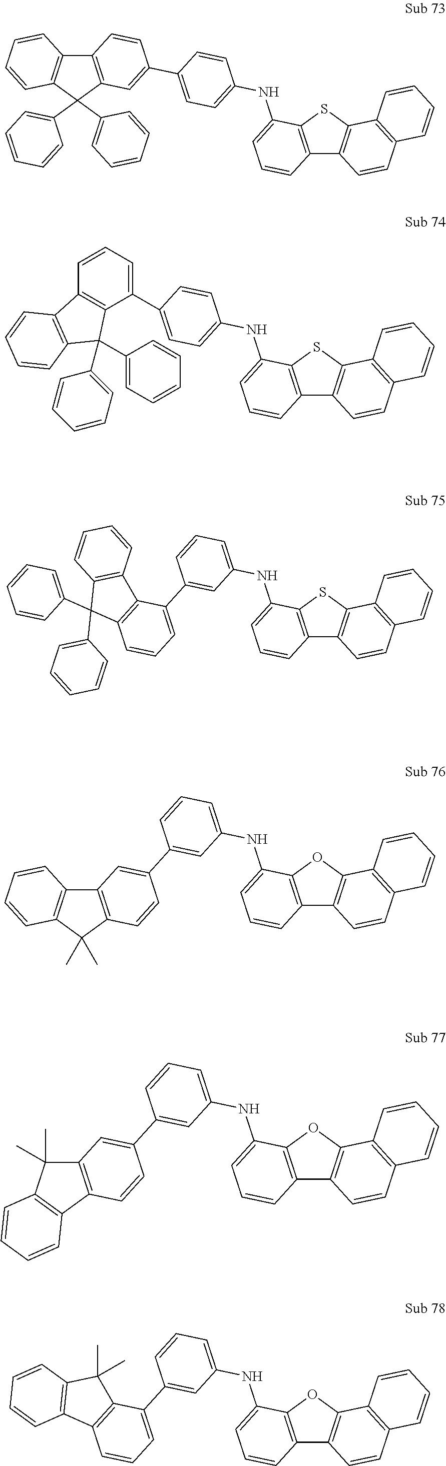 [SMILES: c1ccc(C2(c3ccccc3)c3ccccc3-c3ccc(-c4ccc(Nc5cccc6c5sc5c7ccccc7ccc65)cc4)cc32)cc1 ; c1ccc(C2(c3ccccc3)c3ccccc3-c3cccc(-c4ccc(Nc5cccc6c5sc5c7ccccc7ccc65)cc4)c32)cc1 ; c1ccc(C2(c3ccccc3)c3ccccc3-c3c(-c4cccc(Nc5cccc6c5sc5c7ccccc7ccc65)c4)cccc32)cc1 ; CC1(C)c2ccccc2-c2cc(-c3cccc(Nc4cccc5c4oc4c6ccccc6ccc54)c3)ccc21 ; CC1(C)c2ccccc2-c2ccc(-c3cccc(Nc4cccc5c4oc4c6ccccc6ccc54)c3)cc21 ; CC1(C)c2ccccc2-c2cccc(-c3cccc(Nc4cccc5c4oc4c6ccccc6ccc54)c3)c21]

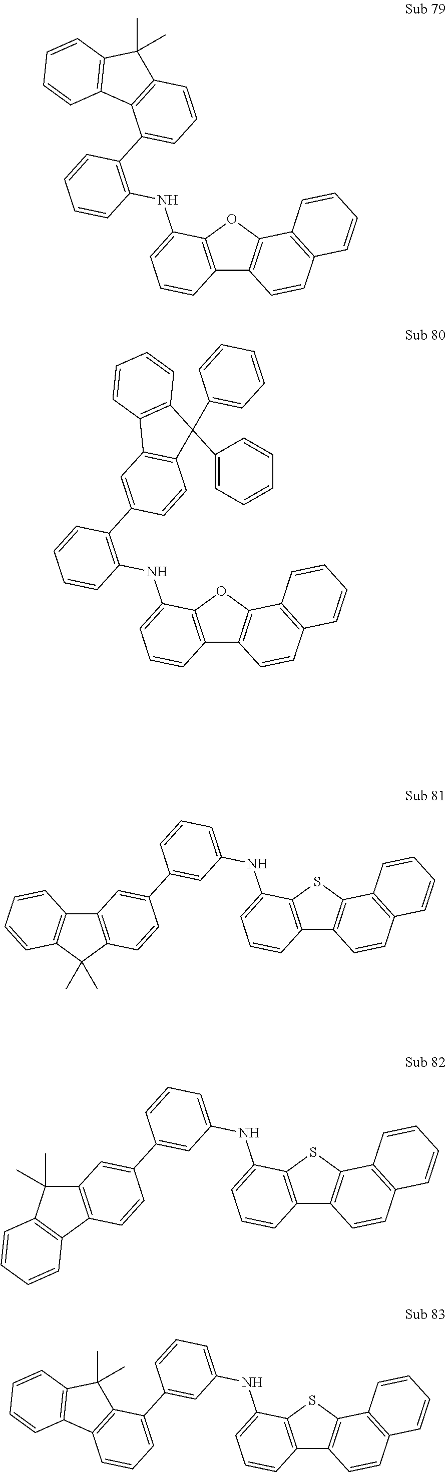 [SMILES: CC1(C)c2ccccc2-c2c(-c3ccccc3Nc3cccc4c3oc3c5ccccc5ccc43)cccc21 ; c1ccc(C2(c3ccccc3)c3ccccc3-c3cc(-c4ccccc4Nc4cccc5c4oc4c6ccccc6ccc54)ccc32)cc1 ; CC1(C)c2ccccc2-c2cc(-c3cccc(Nc4cccc5c4sc4c6ccccc6ccc54)c3)ccc21 ; CC1(C)c2ccccc2-c2ccc(-c3cccc(Nc4cccc5c4sc4c6ccccc6ccc54)c3)cc21 ; CC1(C)c2ccccc2-c2cccc(-c3cccc(Nc4cccc5c4sc4c6ccccc6ccc54)c3)c21]

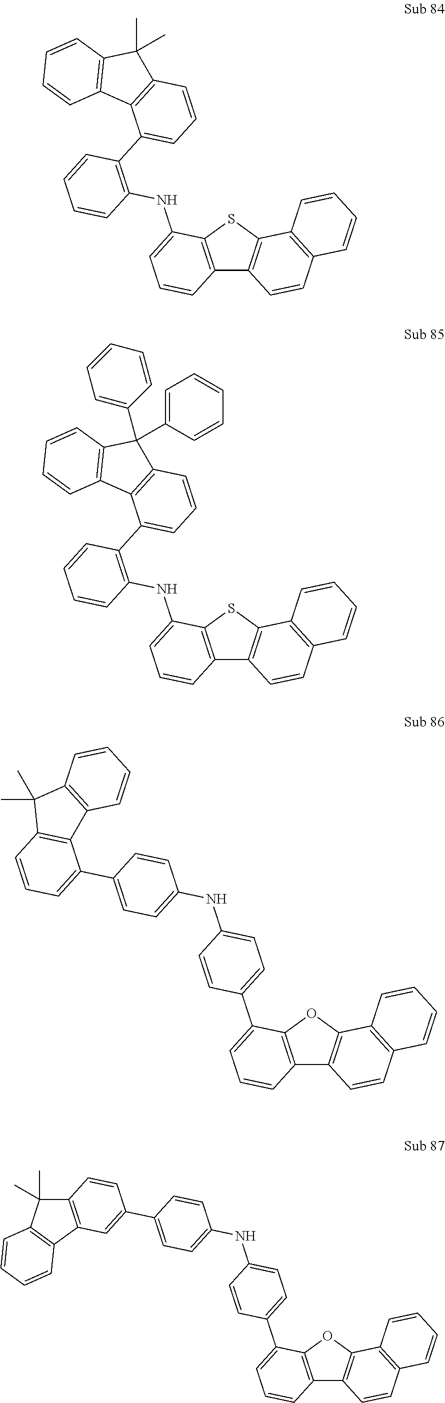 [SMILES: CC1(C)c2ccccc2-c2c(-c3ccccc3Nc3cccc4c3sc3c5ccccc5ccc43)cccc21 ; c1ccc(C2(c3ccccc3)c3ccccc3-c3c(-c4ccccc4Nc4cccc5c4sc4c6ccccc6ccc54)cccc32)cc1 ; CC1(C)c2ccccc2-c2c(-c3ccc(Nc4ccc(-c5cccc6c5oc5c7ccccc7ccc65)cc4)cc3)cccc21 ; CC1(C)c2ccccc2-c2cc(-c3ccc(Nc4ccc(-c5cccc6c5oc5c7ccccc7ccc65)cc4)cc3)ccc21]

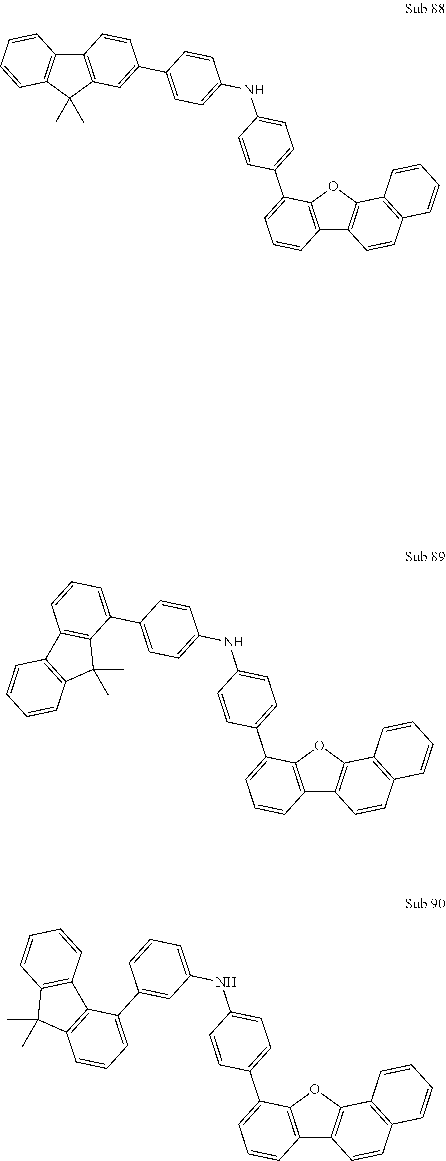 [SMILES: CC1(C)c2ccccc2-c2ccc(-c3ccc(Nc4ccc(-c5cccc6c5oc5c7ccccc7ccc65)cc4)cc3)cc21 ; CC1(C)c2ccccc2-c2cccc(-c3ccc(Nc4ccc(-c5cccc6c5oc5c7ccccc7ccc65)cc4)cc3)c21 ; CC1(C)c2ccccc2-c2c(-c3cccc(Nc4ccc(-c5cccc6c5oc5c7ccccc7ccc65)cc4)c3)cccc21]

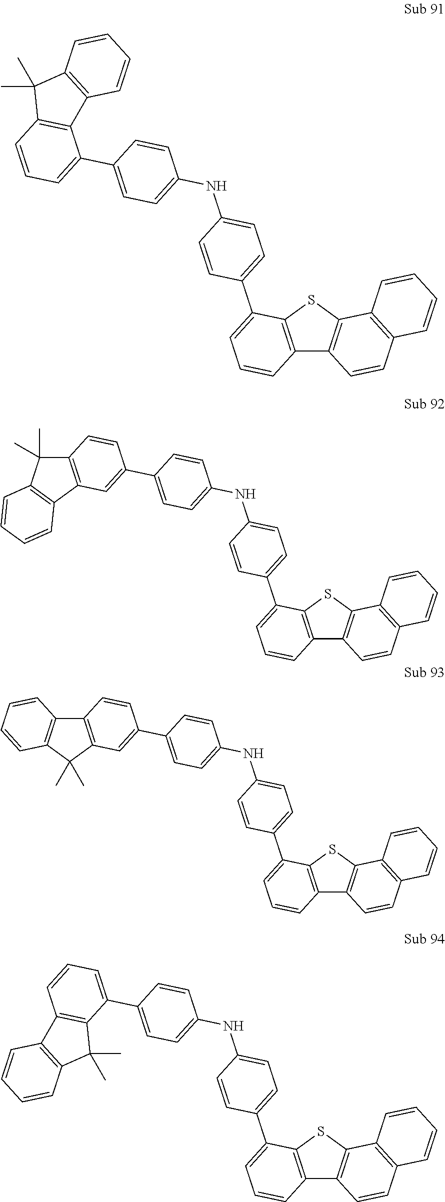 [SMILES: CC1(C)c2ccccc2-c2c(-c3ccc(Nc4ccc(-c5cccc6c5sc5c7ccccc7ccc65)cc4)cc3)cccc21 ; CC1(C)c2ccccc2-c2cc(-c3ccc(Nc4ccc(-c5cccc6c5sc5c7ccccc7ccc65)cc4)cc3)ccc21 ; CC1(C)c2ccccc2-c2ccc(-c3ccc(Nc4ccc(-c5cccc6c5sc5c7ccccc7ccc65)cc4)cc3)cc21 ; CC1(C)c2ccccc2-c2cccc(-c3ccc(Nc4ccc(-c5cccc6c5sc5c7ccccc7ccc65)cc4)cc3)c21]

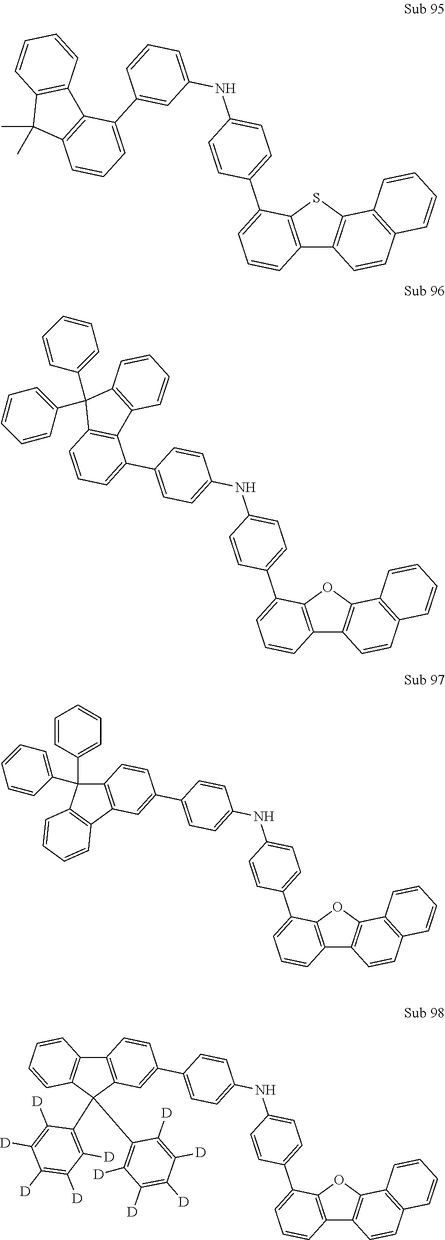 [SMILES: CC1(C)c2ccccc2-c2c(-c3cccc(Nc4ccc(-c5cccc6c5sc5c7ccccc7ccc65)cc4)c3)cccc21 ; c1ccc(C2(c3ccccc3)c3ccccc3-c3c(-c4ccc(Nc5ccc(-c6cccc7c6oc6c8ccccc8ccc76)cc5)cc4)cccc32)cc1 ; c1ccc(C2(c3ccccc3)c3ccccc3-c3cc(-c4ccc(Nc5ccc(-c6cccc7c6oc6c8ccccc8ccc76)cc5)cc4)ccc32)cc1 ; [2H]c1c([2H])c([2H])c(C2(c3c([2H])c([2H])c([2H])c([2H])c3[2H])c3ccccc3-c3ccc(-c4ccc(Nc5ccc(-c6cccc7c6oc6c8ccccc8ccc76)cc5)cc4)cc32)c([2H])c1[2H]]

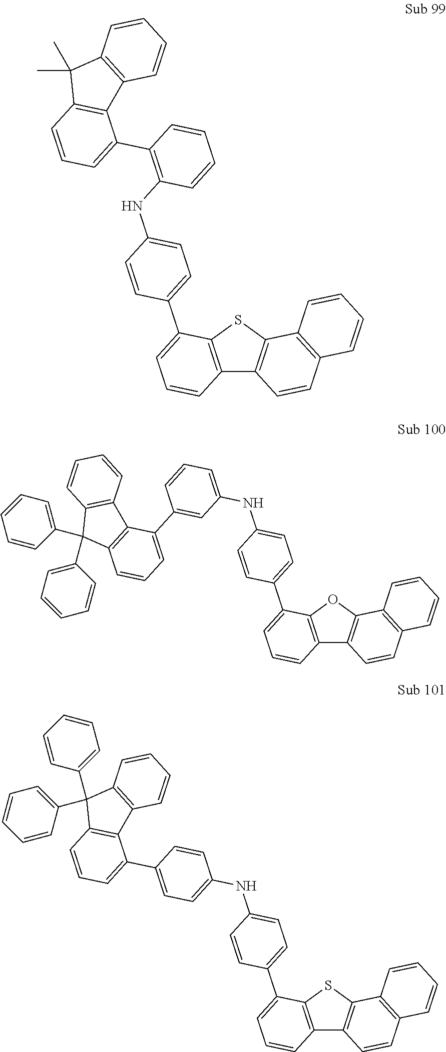 [SMILES: CC1(C)c2ccccc2-c2c(-c3ccccc3Nc3ccc(-c4cccc5c4sc4c6ccccc6ccc54)cc3)cccc21 ; c1ccc(C2(c3ccccc3)c3ccccc3-c3c(-c4cccc(Nc5ccc(-c6cccc7c6oc6c8ccccc8ccc76)cc5)c4)cccc32)cc1 ; c1ccc(C2(c3ccccc3)c3ccccc3-c3c(-c4ccc(Nc5ccc(-c6cccc7c6sc6c8ccccc8ccc76)cc5)cc4)cccc32)cc1]

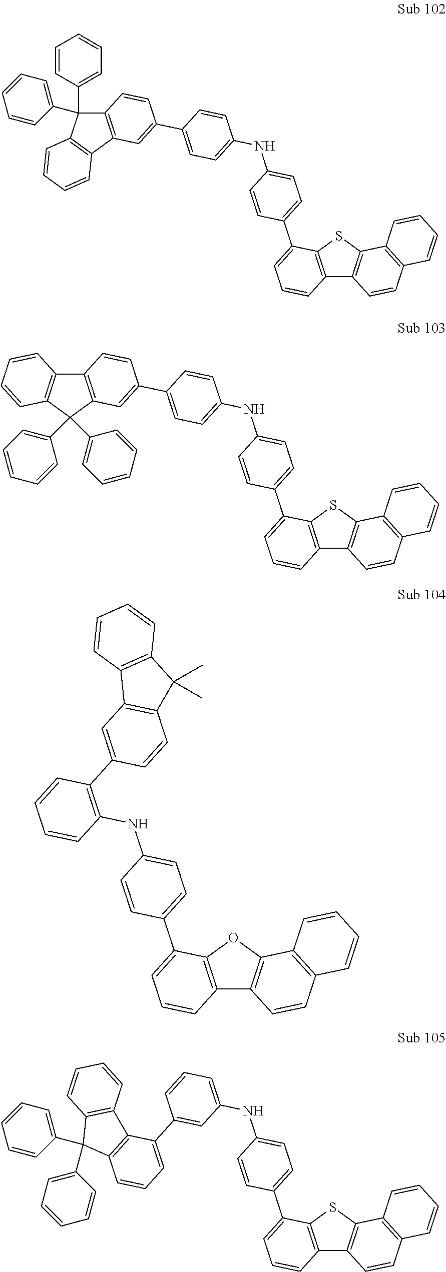 [SMILES: c1ccc(C2(c3ccccc3)c3ccccc3-c3cc(-c4ccc(Nc5ccc(-c6cccc7c6sc6c8ccccc8ccc76)cc5)cc4)ccc32)cc1 ; c1ccc(C2(c3ccccc3)c3ccccc3-c3ccc(-c4ccc(Nc5ccc(-c6cccc7c6sc6c8ccccc8ccc76)cc5)cc4)cc32)cc1 ; CC1(C)c2ccccc2-c2cc(-c3ccccc3Nc3ccc(-c4cccc5c4oc4c6ccccc6ccc54)cc3)ccc21 ; c1ccc(C2(c3ccccc3)c3ccccc3-c3c(-c4cccc(Nc5ccc(-c6cccc7c6sc6c8ccccc8ccc76)cc5)c4)cccc32)cc1]

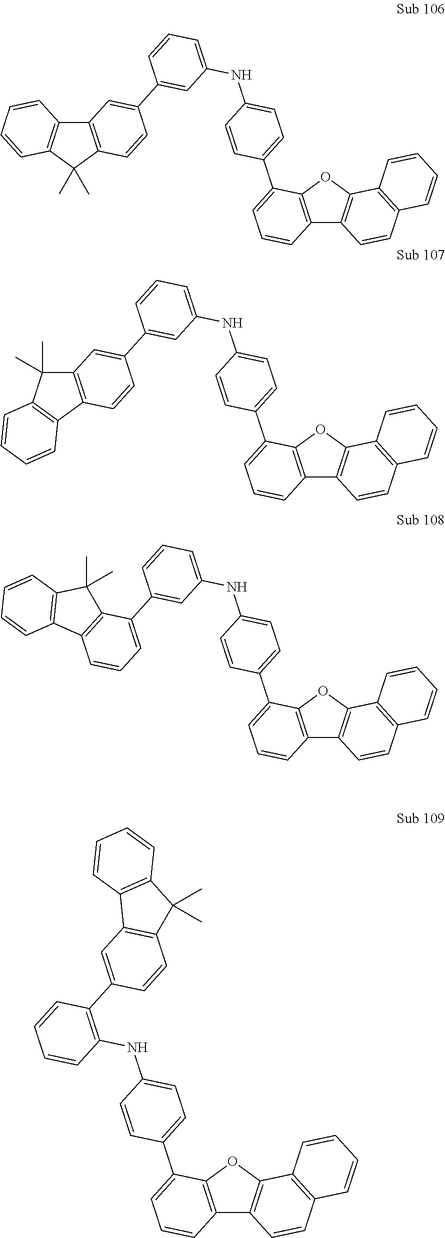 [SMILES: CC1(C)c2ccccc2-c2cc(-c3cccc(Nc4ccc(-c5cccc6c5oc5c7ccccc7ccc65)cc4)c3)ccc21 ; CC1(C)c2ccccc2-c2ccc(-c3cccc(Nc4ccc(-c5cccc6c5oc5c7ccccc7ccc65)cc4)c3)cc21 ; CC1(C)c2ccccc2-c2cccc(-c3cccc(Nc4ccc(-c5cccc6c5oc5c7ccccc7ccc65)cc4)c3)c21 ; CC1(C)c2ccccc2-c2cc(-c3ccccc3Nc3ccc(-c4cccc5c4oc4c6ccccc6ccc54)cc3)ccc21]

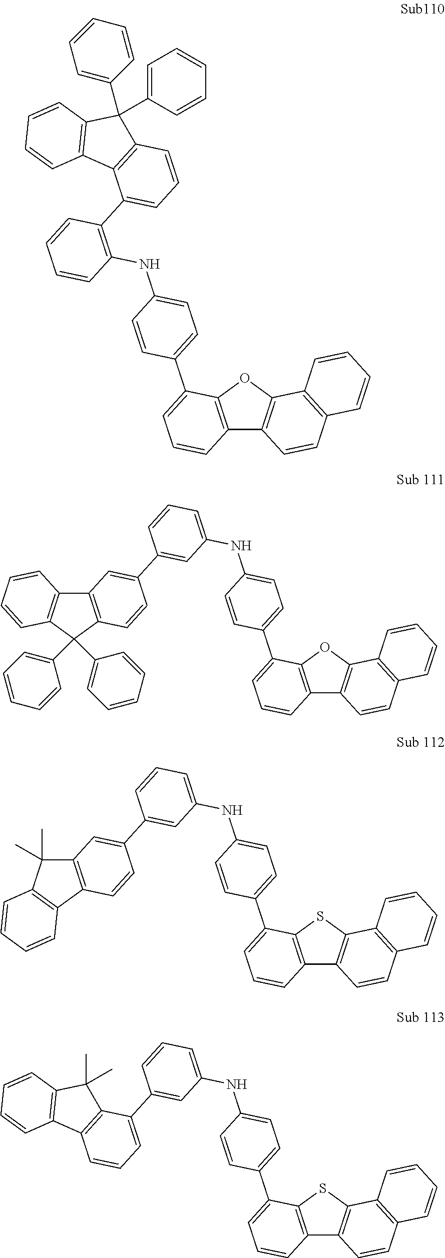 [SMILES: c1ccc(C2(c3ccccc3)c3ccccc3-c3c(-c4ccccc4Nc4ccc(-c5cccc6c5oc5c7ccccc7ccc65)cc4)cccc32)cc1 ; c1ccc(C2(c3ccccc3)c3ccccc3-c3cc(-c4cccc(Nc5ccc(-c6cccc7c6oc6c8ccccc8ccc76)cc5)c4)ccc32)cc1 ; CC1(C)c2ccccc2-c2ccc(-c3cccc(Nc4ccc(-c5cccc6c5sc5c7ccccc7ccc65)cc4)c3)cc21 ; CC1(C)c2ccccc2-c2cccc(-c3cccc(Nc4ccc(-c5cccc6c5sc5c7ccccc7ccc65)cc4)c3)c21]

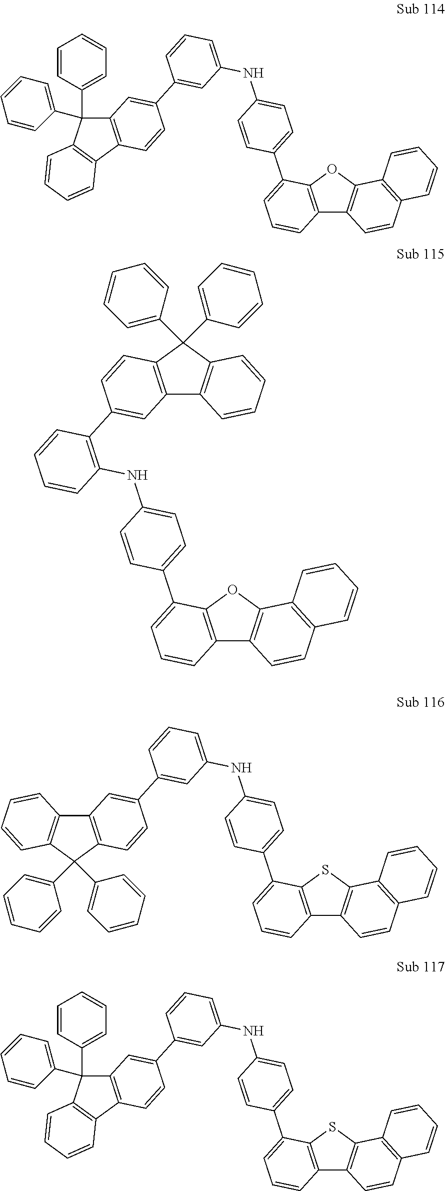 [SMILES: c1ccc(C2(c3ccccc3)c3ccccc3-c3ccc(-c4cccc(Nc5ccc(-c6cccc7c6oc6c8ccccc8ccc76)cc5)c4)cc32)cc1 ; c1ccc(C2(c3ccccc3)c3ccccc3-c3cc(-c4ccccc4Nc4ccc(-c5cccc6c5oc5c7ccccc7ccc65)cc4)ccc32)cc1 ; c1ccc(C2(c3ccccc3)c3ccccc3-c3cc(-c4cccc(Nc5ccc(-c6cccc7c6sc6c8ccccc8ccc76)cc5)c4)ccc32)cc1 ; c1ccc(C2(c3ccccc3)c3ccccc3-c3ccc(-c4cccc(Nc5ccc(-c6cccc7c6sc6c8ccccc8ccc76)cc5)c4)cc32)cc1]

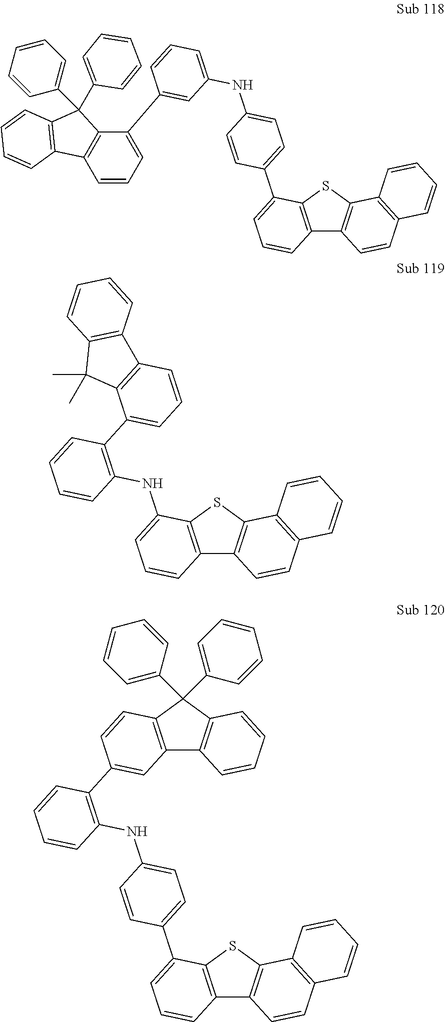 [SMILES: c1ccc(C2(c3ccccc3)c3ccccc3-c3cccc(-c4cccc(Nc5ccc(-c6cccc7c6sc6c8ccccc8ccc76)cc5)c4)c32)cc1 ; CC1(C)c2ccccc2-c2cccc(-c3ccccc3Nc3cccc4c3sc3c5ccccc5ccc43)c21 ; c1ccc(C2(c3ccccc3)c3ccccc3-c3cc(-c4ccccc4Nc4ccc(-c5cccc6c5sc5c7ccccc7ccc65)cc4)ccc32)cc1]

Table 1 below shows FD-MS values of Products Sub 1 to Sub 120.

TABLE 1

| Compound | FD-MS | Compound | FD-MS |
|---|---|---|---|
| Sub 1 | m/z = 424.38 (C₃₁H₂₃NO = 425.18) | Sub 2 | m/z = 424.38 (C₃₁H₂₃NO = 425.18) |
| Sub 3 | m/z = 424.38 (C₃₁H₂₃NO = 425.18) | Sub 4 | m/z = 424.38 (C₃₁H₂₃NO = 425.18) |
| Sub 5 | m/z = 500.44 (C₃₇H₂₇NO = 501.21) | Sub 6 | m/z = 440.36 (C₃₁H₂₃NS = 441.16) |
| Sub 7 | m/z = 440.36 (C₃₁H₂₃NS = 441.16) | Sub 8 | m/z = 440.36 (C₃₁H₂₃NS = 441.16) |
| Sub 9 | m/z = 440.36 (C₃₁H₂₃NS = 441.16) | Sub 10 | m/z = 516.38 (C₃₇H₂₇NS = 517.19) |
| Sub 11 | m/z = 557.47 (C₄₁H₁₈D₉NO = 558.27) | Sub 12 | m/z = 548.51 (C₄₁H₂₇NO = 549.21) |
| Sub 13 | m/z = 548.51 (C₄₁H₂₇NO = 549.21) | Sub 14 | m/z = 548.71 (C₄₁H₂₇NO = 549.21) |
| Sub 15 | m/z = 624.44 (C₄₇H₃₁NO = 625.24) | Sub 16 | m/z = 564.59 (C₄₁H₂₇NS = 565.19) |
| Sub 17 | m/z = 564.49 (C₄₁H₂₇NS = 565.19) | Sub 18 | m/z = 564.69 (C₄₁H₂₇NS = 565.19) |
| Sub 19 | m/z = 576.64 (C₄₃H₃₁NO = 577.24) | Sub 20 | m/z = 640.62 (C₄₇H₃₁NS = 641.22) |
| Sub 21 | m/z = 500.61 (C₃₇H₂₇NO = 501.21) | Sub 22 | m/z = 504.63 (C₃₇H₂₃D₄NO = 505.23) |
| Sub 23 | m/z = 500.71 (C₃₇H₂₇NO = 501.21) | Sub 24 | m/z = 500.61 (C₃₇H₂₇NO = 501.21) |
| Sub 25 | m/z = 500.61 (C₃₇H₂₇NO = 501.21) | Sub 26 | m/z = 500.61 (C₃₇H₂₇NO = 501.21) |
| Sub 27 | m/z = 516.39 (C₃₇H₂₇NS = 517.19) | Sub 28 | m/z = 516.49 (C₃₇H₂₇NS = 517.19) |
| Sub 29 | m/z = 516.39 (C₃₇H₂₇NS = 517.19) | Sub 30 | m/z = 516.39 (C₃₇H₂₇NS = 517.19) |
| Sub 31 | m/z = 624.44 (C₄₇H₃₁NO = 625.24) | Sub 32 | m/z = 624.74 (C₄₇H₃₁NO = 625.24) |
| Sub 33 | m/z = 516.69 (C₃₇H₂₇NS = 517.19) | Sub 34 | m/z = 507.55 (C₃₇H₂₀D₇NO = 508.25) |
| Sub 35 | m/z = 624.74 (C₄₇H₃₁NO = 625.24) | Sub 36 | m/z = 653.5 (C₄₇H₁₈D₁₃NS = 654.30) |
| Sub 37 | m/z = 640.52 (C₄₇H₃₁NS = 641.22) | Sub 38 | m/z = 640.52 (C₄₇H₃₁NS = 641.22) |
| Sub 39 | m/z = 640.52 (C₄₇H₃₁NS = 641.22) | Sub 40 | m/z = 500.51 (C₃₇H₂₇NO = 501.21) |
| Sub 41 | m/z = 500.71 (C₃₇H₂₇NO = 501.21) | Sub 42 | m/z = 500.51 (C₃₇H₂₇NO = 501.21) |
| Sub 43 | m/z = 516.49 (C₃₇H₂₇NS = 517.19) | Sub 44 | m/z = 624.54 (C₄₇H₃₁NO = 625.24) |
| Sub 45 | m/z = 516.49 (C₃₇H₂₇NS = 517.19) | Sub 46 | m/z = 624.74 (C₄₇H₃₁NO = 625.24) |
| Sub 47 | m/z = 640.52 (C₄₇H₃₁NS = 641.22) | Sub 48 | m/z = 624.54 (C₄₇H₃₁NO = 625.24) |
| Sub 49 | m/z = 624.54 (C₄₇H₃₁NO = 625.24) | Sub 50 | m/z = 624.54 (C₄₇H₃₁NO = 625.24) |
| Sub 51 | m/z = 640.52 (C₄₇H₃₁NS = 641.22) | Sub 52 | m/z = 500.51 (C₃₇H₂₇NO = 501.21) |
| Sub 53 | m/z = 500.51 (C₃₇H₂₇NO = 501.21) | Sub 54 | m/z = 640.52 (C₄₇H₃₁NS = 641.22) |
| Sub 55 | m/z = 640.52 (C₄₇H₃₁NS = 641.22) | Sub 56 | m/z = 520.63 (C₃₇H₇D₂₀NO = 521.33) |
| Sub 57 | m/z = 640.52 (C₄₇H₃₁NS = 641.22) | Sub 58 | m/z = 640.42 (C₄₇H₃₁NS = 641.22) |
| Sub 59 | m/z = 500.51 (C₃₇H₂₇NO = 501.21) | Sub 60 | m/z = 500.51 (C₃₇H₂₇NO = 501.21) |
| Sub 61 | m/z = 516.49 (C₃₇H₂₇NS = 517.19) | Sub 62 | m/z = 624.54 (C₄₇H₃₁NO = 625.24) |
| Sub 63 | m/z = 516.49 (C₃₇H₂₇NS = 517.19) | Sub 64 | m/z = 516.69 (C₃₇H₂₇NS = 517.19) |
| Sub 65 | m/z = 624.44 (C₄₇H₃₁NO = 625.24) | Sub 66 | m/z = 647.56 (C₄₇H₂₄D₇NS = 648.26) |
| Sub 67 | m/z = 624.54 (C₄₇H₃₁NO = 625.24) | Sub 68 | m/z = 624.54 (C₄₇H₃₁NO = 625.24) |
| Sub 69 | m/z = 624.54 (C₄₇H₃₁NO = 625.24) | Sub 70 | m/z = 624.54 (C₄₇H₃₁NO = 625.24) |
| Sub 71 | m/z = 647.56 (C₄₇H₂₄D₇NS = 648.26) | Sub 72 | m/z = 640.52 (C₄₇H₃₁NS = 641.22) |
| Sub 73 | m/z = 640.52 (C₄₇H₃₁NS = 641.22) | Sub 74 | m/z = 640.52 (C₄₇H₃₁NS = 641.22) |
| Sub 75 | m/z = 640.52 (C₄₇H₃₁NS = 641.22) | Sub 76 | m/z = 500.51 (C₃₇H₂₇NO = 501.21) |
| Sub 77 | m/z = 500.51 (C₃₇H₂₇NO = 501.21) | Sub 78 | m/z = 500.51 (C₃₇H₂₇NO = 501.21) |
| Sub 79 | m/z = 500.51 (C₃₇H₂₇NO = 501.21) | Sub 80 | m/z = 640.52 (C₄₇H₃₁NS = 641.22) |
| Sub 81 | m/z = 516.49 (C₃₇H₂₇NS = 517.19) | Sub 82 | m/z = 516.49 (C₃₇H₂₇NS = 517.19) |
| Sub 83 | m/z = 516.39 (C₃₇H₂₇NS = 517.19) | Sub 84 | m/z = 516.49 (C₃₇H₂₇NS = 517.19) |
| Sub 85 | m/z = 640.52 (C₄₇H₃₁NS = 641.22) | Sub 86 | m/z = 576.54 (C₄₃H₃₁NO = 577.24) |
| Sub 87 | m/z = 576.54 (C₄₃H₃₁NO = 577.24) | Sub 88 | m/z = 576.54 (C₄₃H₃₁NO = 577.24) |
| Sub 89 | m/z = 576.54 (C₄₃H₃₁NO = 577.24) | Sub 90 | m/z = 576.54 (C₄₃H₃₁NO = 577.24) |
| Sub 91 | m/z = 592.52 (C₄₃H₃₁NS = 593.22) | Sub 92 | m/z = 592.52 (C₄₃H₃₁NS = 593.22) |
| Sub 93 | m/z = 592.52 (C₄₃H₃₁NS = 593.22) | Sub 94 | m/z = 592.52 (C₄₃H₃₁NS = 593.22) |
| Sub 95 | m/z = 592.72 (C₄₃H₃₁NS = 593.22) | Sub 96 | m/z = 700.57 (C₅₃H₃₅NO = 701.27) |
| Sub 97 | m/z = 700.57 (C₅₃H₃₅NO = 701.27) | Sub 98 | m/z = 710.63 (C₅₃H₂₅D₁₀NO = 711.33) |
| Sub 99 | m/z = 592.52 (C₄₃H₃₁NS = 593.22) | Sub 100 | m/z = 700.57 (C₅₃H₃₅NO = 701.27) |
| Sub 101 | m/z = 716.55 (C₅₃H₃₅NS = 717.25) | Sub 102 | m/z = 716.55 (C₅₃H₃₅NS = 717.25) |
| Sub 103 | m/z = 716.55 (C₅₃H₃₅NS = 717.25) | Sub 104 | m/z = 576.54 (C₄₃H₃₁NO = 577.24) |
| Sub 105 | m/z = 716.55 (C₅₃H₃₅NS = 717.25) | Sub 106 | m/z = 576.54 (C₄₃H₃₁NO = 577.24) |
| Sub 107 | m/z = 576.54 (C₄₃H₃₁NO = 577.24) | Sub 108 | m/z = 576.54 (C₄₃H₃₁NO = 577.24) |
| Sub 109 | m/z = 592.52 (C₄₃H₃₁NS = 593.22) | Sub 110 | m/z = 700.57 (C₅₃H₃₅NO = 701.27) |
| Sub 111 | m/z = 700.57 (C₅₃H₃₅NO = 701.27) | Sub 112 | m/z = 592.72 (C₄₃H₃₁NS = 593.22) |
| Sub 113 | m/z = 592.52 (C₄₃H₃₁NS = 593.22) | Sub 114 | m/z = 700.57 (C₅₃H₃₅NO = 701.27) |
| Sub 115 | m/z = 700.57 (C₅₃H₃₅NO = 701.27) | Sub 116 | m/z = 716.55 (C₅₃H₃₅NS = 717.25) |
| Sub 117 | m/z = 716.55 (C₅₃H₃₅NS = 717.25) | Sub 118 | m/z = 716.55 (C₅₃H₃₅NS = 717.25) |
| Sub 119 | m/z = 516.49 (C₃₇H₂₇NS = 517.19) | Sub 120 | m/z = 716.55 (C₅₃H₃₅NS = 717.25) |

Hereinafter, synthesis examples of some of the compounds belonging to Sub A will be described.

### I-1 Synthesis Example of Sub 5

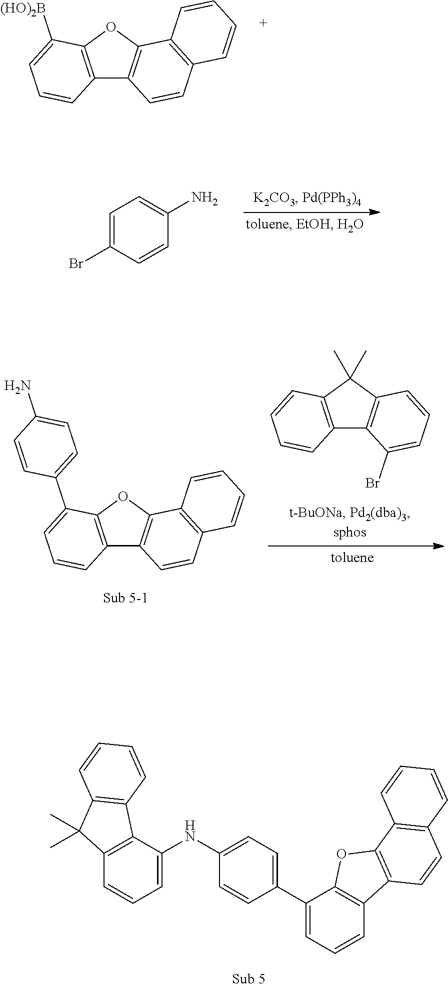 [SMILES: OB(O)c1cccc2c1oc1c3ccccc3ccc21 ; Nc1ccc(Br)cc1 ; Nc1ccc(-c2cccc3c2oc2c4ccccc4ccc32)cc1 ; CC1(C)c2ccccc2-c2c(Br)cccc21 ; CC1(C)c2ccccc2-c2c(Nc3ccc(-c4cccc5c4oc4c6ccccc6ccc54)cc3)cccc21]

### 1-1) Synthesis of Sub 5-1

Naphtho[1,2-b]benzofuran-10-ylboronic acid (100.0 g, 381.6 mmol), 4-bromoaniline (65.64 g, 381.6 mmol), K₂CO₃ (105.5 g, 763.2 mmol), Pd(PPh₃)₄(8.82 g, 7.63 mmol), toluene (1000 mL), ethanol (350 mL), and water (350 mL) were added, stirred, and refluxed in a 5 L flask under nitrogen atmosphere. After the reaction was finished, an organic layer was extracted by using toluene and water. The extracted solution was treated with MgSO₄ to remove remaining moisture, concentrated under reduced pressure, then purified by using a column chromatography method, and then recrystallized to obtain Sub 5-1 of 90.9 g (yield 77%).

### 1-2) Synthesis of Sub 5

4-bromo-9,9-dimethyl-9H-fluorene (40.0 g, 146.4 mmol), Sub 5-1 (45.3 g, 146.4 mmol), t-BuONa (28.1 g, 292.9 mmol), Pd₂(dba)₃ (2.68 g, 2.93 mmol), sphos (2.41 g, 5.86 mmol), and toluene (800 mL) were added, stirred, and refluxed in a 3 L flask under nitrogen atmosphere. After the reaction was finished, an organic layer was extracted by using toluene and water. The extracted solution was treated with MgSO₄ to remove remaining moisture, concentrated under reduced pressure, then purified by using a column chromatography method, and then recrystallized to obtain Sub 5 of 120.5 g (yield 82%).

### I-2 Synthesis Example of Sub 15

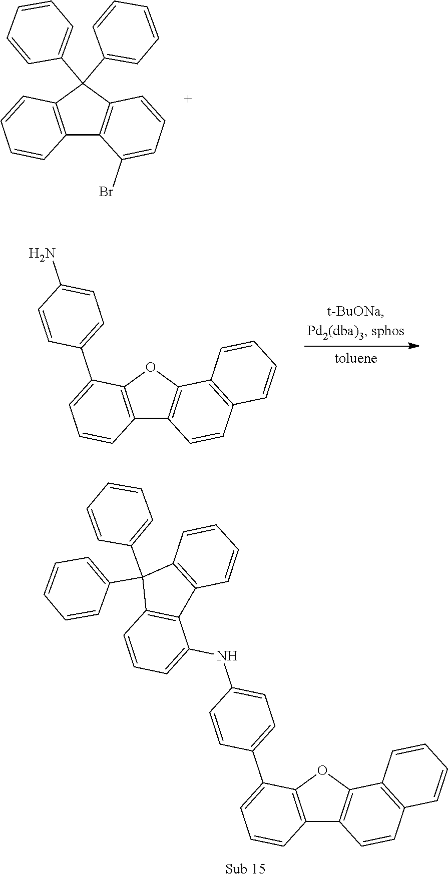

### 2-1) Synthesis of Sub 15

Sub 15 of 53.5 g (yield 85%) was obtained by the synthesis and purification using the manufacturing method of Sub 5 except that 4-bromo-9,9-diphenyl-9H-fluorene (40.0 g, 100.7 mmol) was used instead of 4-bromo-9,9-dimethyl-9H-fluorene.

### I-3 Synthesis Example of Sub 86

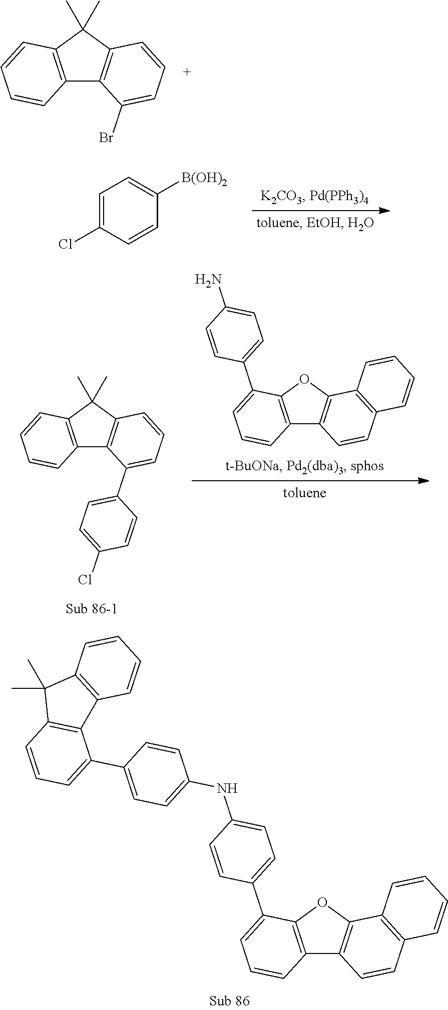

### 3-1) Synthesis of Sub 86-1

Sub 86-1 of 85.9 g (77% yield) was obtained by the synthesis and purification using the manufacturing method of Sub 5-1 except that 4-bromo-9,9-dimethyl-9H-fluorene (100.0 g, 366.1 mmol) and (4-chlorophenyl)boronic acid (21.5 g, 107.5 mmol) were used.

### 3-2) Synthesis of Sub 86

Sub 86 of 64.1 g (78% yield) was obtained by the synthesis and purification using the manufacturing method of Sub 5 except that Sub 86-1 (40.0 g, 131.2 mmol) and Sub 5-1 (40.6 g, 131.2 mmol) were used.

### I-4 Synthesis Example of Sub 42

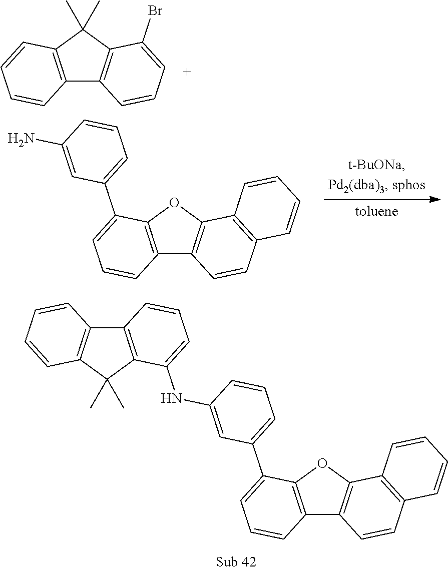

### 4-1) Synthesis of Sub 42

Sub 42 of 60.7 g (yield 75%) was obtained by the synthesis and purification using the manufacturing method of Sub 5 except that 1-bromo-9,9-dimethyl-9H-fluorene (35.3 g, 129.3 mmol) and 3-(naphtho[1,2-b]benzofuran-10-yl)aniline (40.0 g, 129.3 mmol) were used.

### I-5 Synthesis Example of Sub 18

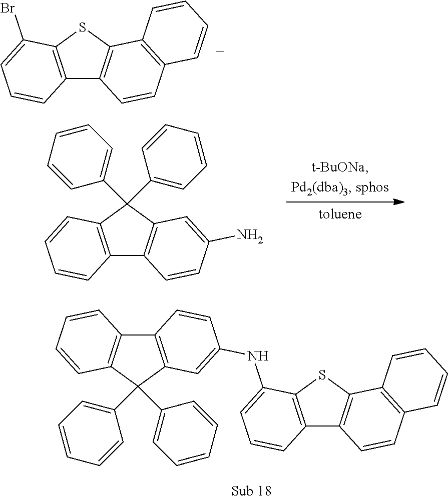

### 5-1) Synthesis of Sub 18

Sub 18 of 51.6 g (yield 76%) was obtained by the synthesis and purification using the manufacturing method of Sub 5 except that 10-bromobenzo[b]naphtho[2,1-d]thiophene (37.6 g, 120.0 mmol) and 9,9-diphenyl-9H-fluoren-2-amine (40.0 g, 120.0 mmol) were used.

### I-6 Synthesis Example of Sub 10

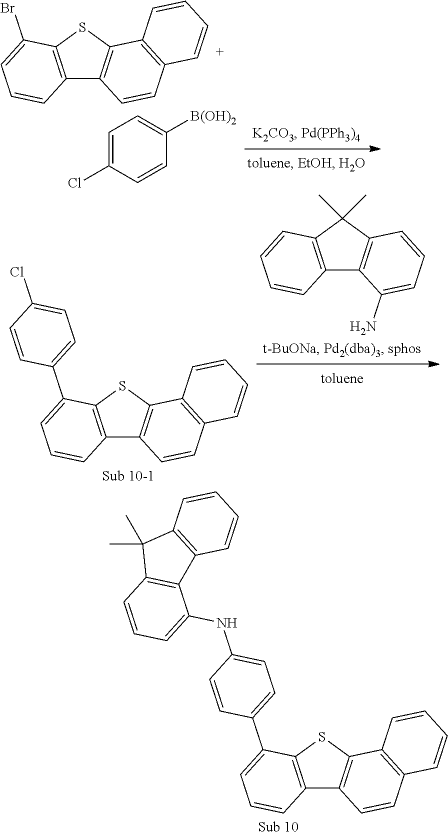

### 6-1) Synthesis of Sub 10-1

Sub 10-1 of 82.6 g (75% yield) was obtained by the synthesis and purification using the manufacturing method of Sub 5-1 except that 10-bromobenzo[b]naphtho[2,1-d]thiophene (100.0 g, 319.3 mmol) and (4-chlorophenyl)boronic acid (49.9 g, 319.3 mmol) were used.

### 6-2) Synthesis of Sub 10

Sub 10 of 52.5 g (yield 80%) was obtained by the synthesis and purification using the manufacturing method of Sub 5 except that Sub 10-1 (40.0 g, 116.0 mmol) and 9,9-dimethyl-9H-fluoren-4-amine (24.3 g, 116.0 mmol) were used.

### I-7 Synthesis Example of Sub 82

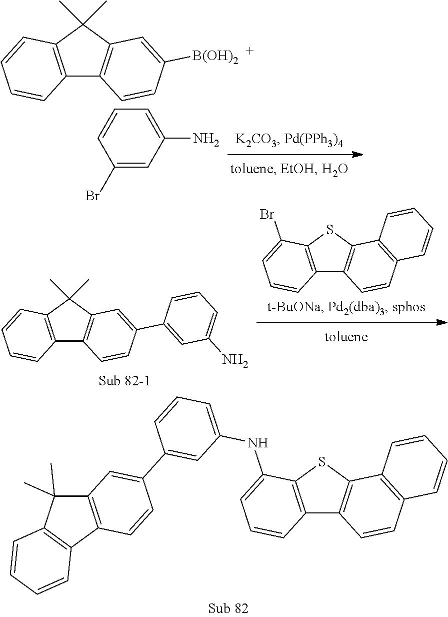

### 7-1) Synthesis of Sub 82-1

Sub 82-1 of 83.9 g (70% yield) was obtained by the synthesis and purification using the manufacturing method of Sub 5-1 except that (9,9-dimethyl-9H-fluoren-2-yl)boronic acid (100.0 g, 420.0 mmol) and 3-bromoaniline (72.3 g, 420.0 mmol) were used.

### 7-2) Synthesis of Sub 82

Sub 82 of 48.9 g (yield 74%) was obtained by the synthesis and purification using the manufacturing method of Sub 5 except that Sub 82-1 (36.4 g, 127.7 mmol) and 10-bromobenzo[b]naphtho[2,1-d]thiophene (40.0 g, 127.7 mmol) were used.

### I-8 Synthesis Example of Sub 17

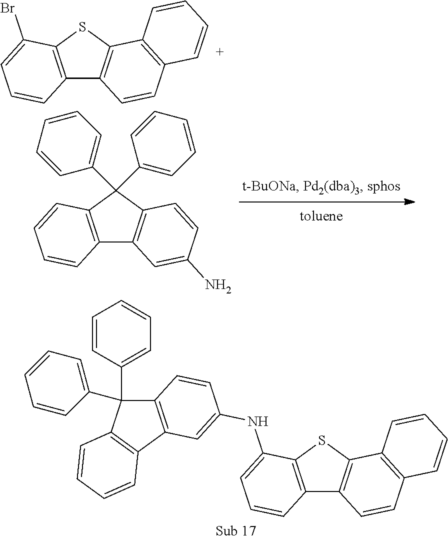

### 8-1) Synthesis of Sub 17

Sub 17 of 46.0 g (yield 74%) was obtained by the synthesis and purification using the manufacturing method of Sub 5 except that 10-bromobenzo[b]naphtho[2,1-d]thiophene (37.6 g, 120.0 mmol) and 9,9-diphenyl-9H-fluoren-3-amine (40.0 g, 120.0 mmol) were used.

### II. Synthesis Example of Final Compound

The final compound (is referred to as “product” and is the same as Formula 1-1), which is a compound belonging to Formula 1-1 in which Ar₁ in Chemical Formula 1 of the present invention is selected as Chemical Formula 2, may be synthesized as represented by Reaction Formula 2 below, but is not limited thereto. (In Reaction Formula 2 below, X═O or S, Hal=Br or Cl)

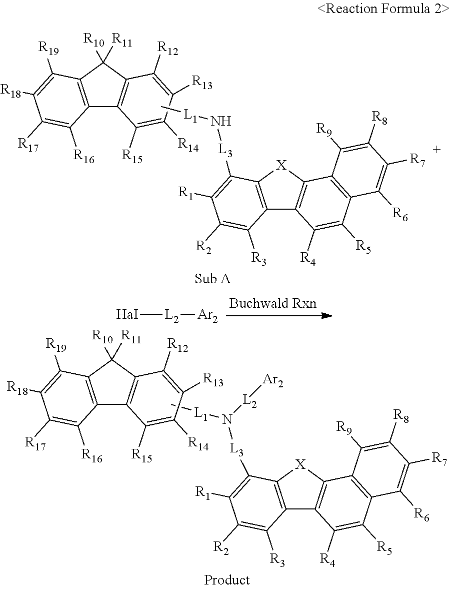

### II-1 Synthesis of Compound P 44

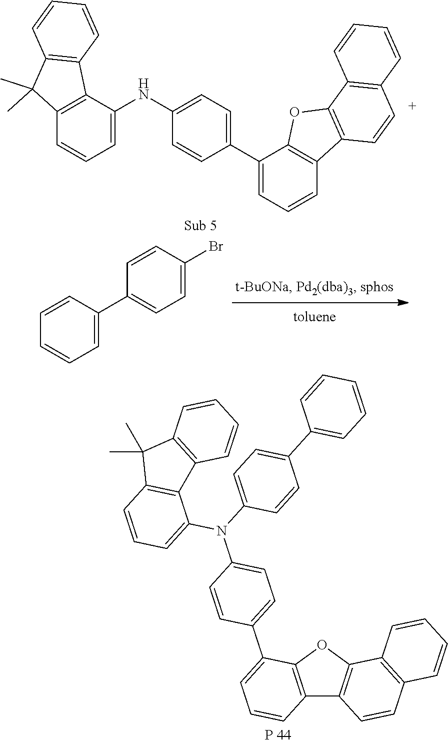 [SMILES: CC1(C)c2ccccc2-c2c(Nc3ccc(-c4cccc5c4oc4c6ccccc6ccc54)cc3)cccc21 ; Brc1ccc(-c2ccccc2)cc1 ; CC1(C)c2ccccc2-c2c(N(c3ccc(-c4ccccc4)cc3)c3ccc(-c4cccc5c4oc4c6ccccc6ccc54)cc3)cccc21]

Sub 5 (8.0 g, 15.95 mmol), 4-bromo-1,1′-biphenyl (4.09 g, 17.54 mmol), t-BuONa (3.07 g, 31.90 mmol), Pd₂(dba)₃ (0.29 g, 0.32 mmol), sphos (0.26 g, 0.64 mmol), and toluene (100 mL) were added, stirred, and refluxed in a 500 mL flask under nitrogen atmosphere. After the reaction was finished, an organic layer was extracted by using toluene and water. The extracted solution was treated with MgSO₄ to remove remaining moisture, concentrated under reduced pressure, then purified by using a column chromatography method, and then recrystallized to obtain Compound P 44 of 7.30 g (yield 70%).

### II-2 Synthesis of Compound P 45

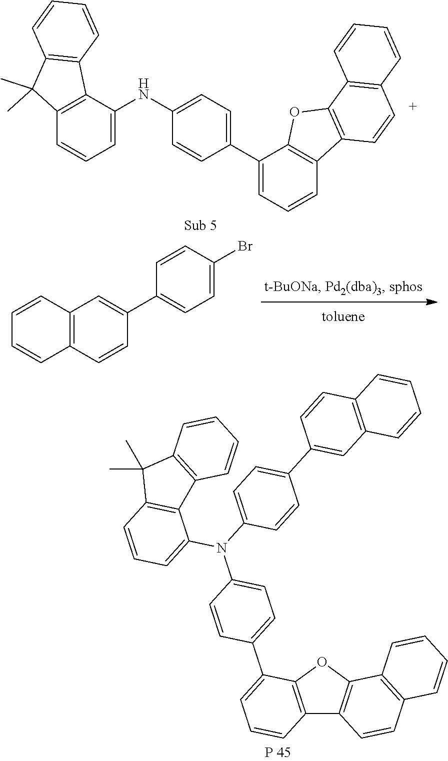 [SMILES: CC1(C)c2ccccc2-c2c(Nc3ccc(-c4cccc5c4oc4c6ccccc6ccc54)cc3)cccc21 ; Brc1ccc(-c2ccc3ccccc3c2)cc1 ; CC1(C)c2ccccc2-c2c(N(c3ccc(-c4ccc5ccccc5c4)cc3)c3ccc(-c4cccc5c4oc4c6ccccc6ccc54)cc3)cccc21]

Compound P 45 of 7.52 g (yield 67%) was obtained by synthesis and purification in the same manner as the manufacturing method of Compound P 44 except that 2-(4-bromophenyl)naphthalene (4.97 g, 17.54 mmol) was used instead of 4-bromo-1,1′-biphenyl.

### II-3 Synthesis of Compound P29

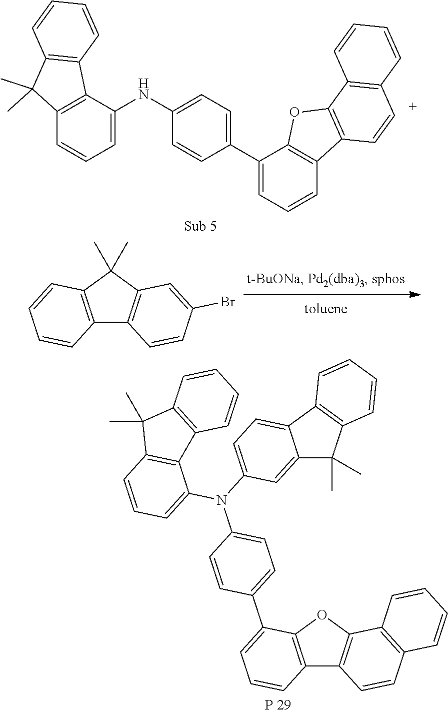

Compound P 29 of 7.97 g (yield 72%) was obtained by synthesis and purification using the manufacturing method of Compound P 44 except that 2-bromo-9,9-dimethyl-9H-fluorene (4.79 g, 17.54 mmol) was used instead of 4-bromo-1,1′-biphenyl.

### II-4 Synthesis of Compound P 31

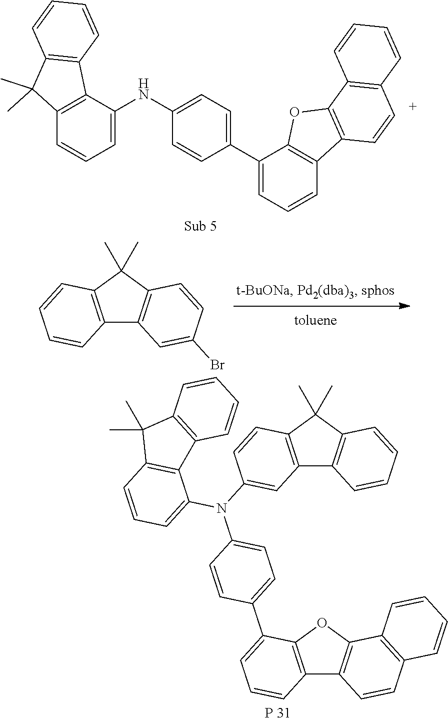

Compound P 31 of 7.19 g (yield 65%) was obtained by synthesis and purification using the manufacturing method of Compound P 44 except that 3-bromo-9,9-dimethyl-9H-fluorene (4.79 g, 17.54 mmol) was used instead of 4-bromo-1,1′-biphenyl.

### II-5 Synthesis of Compound 387

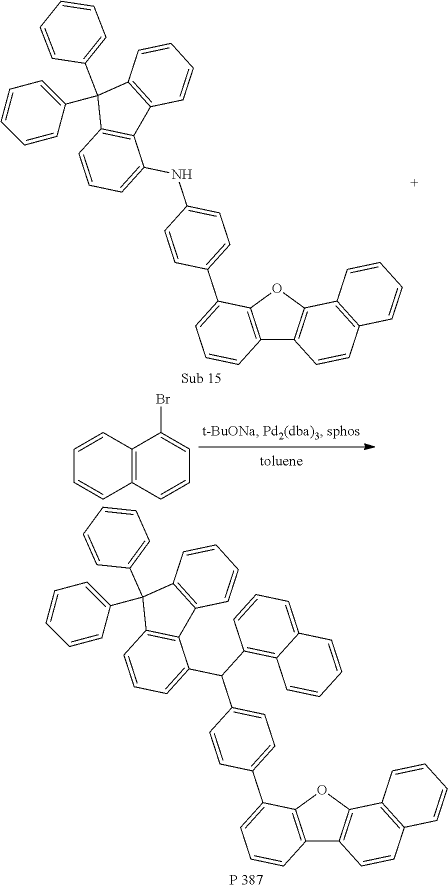

Compound P 387 of 7.11 g (yield 74%) was obtained by synthesis and purification in the same manner as the manufacturing method of Compound P 44 except that Sub 15 (8.0 g, 12.78 mmol) and 1-bromonaphthalene (2.91 g, 14.06 mmol) were used.

### II-6 Synthesis of Compound P 71

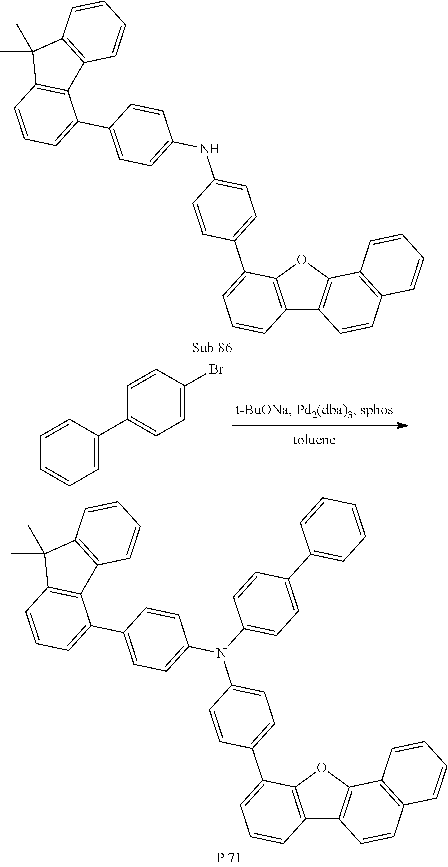

Compound P 71 of 7.88 g (yield 78%) was obtained by synthesis and purification in the same manner as the manufacturing method of Compound P 44 except that Sub 86 (8.00 g, 13.85 mmol) and 4-bromo-1,1′-biphenyl (3.55 g, 15.23 mmol) were used.

### II-7 Synthesis of Compound P 117

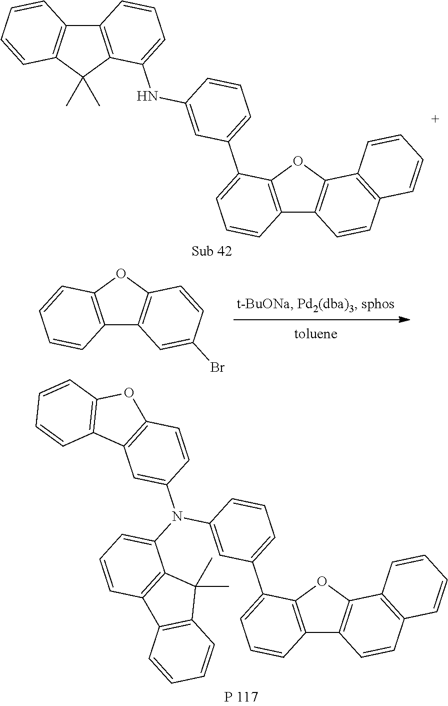

Compound P 117 of 7.24 g (yield 68%) was obtained by synthesis and purification in the same manner as the manufacturing method of Compound P 44 except that Sub 42 (8.00 g, 15.95 mmol) and 2-bromodibenzo[b,d]furan (4.34 g, 17.54 mmol) were used.

### II-8 Synthesis of Compound P 641

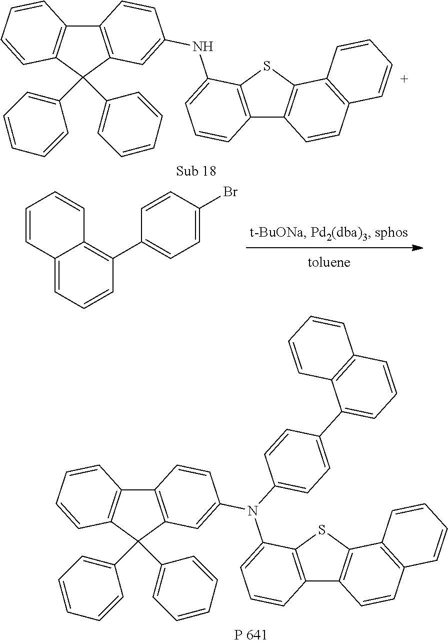

Compound P 641 of 6.14 g (yield 65%) was obtained by synthesis and purification in the same manner as the manufacturing method of Compound P 44 except that Sub 18 (8.00 g, 14.14 mmol) and 1-(4-bromophenyl)naphthalene (4.41 g, 15.56 mmol) were used.

### II-9 Synthesis of Compound P 228

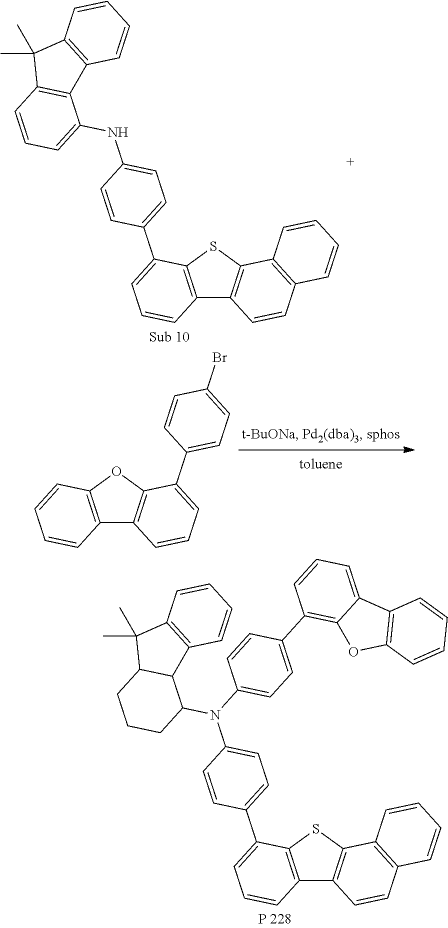 [SMILES: CC1(C)c2ccccc2-c2c(Nc3ccc(-c4cccc5c4sc4c6ccccc6ccc54)cc3)cccc21 ; Brc1ccc(-c2cccc3c2oc2ccccc23)cc1 ; CC1(C)c2ccccc2C2C(N(c3ccc(-c4cccc5c4oc4ccccc45)cc3)c3ccc(-c4cccc5c4sc4c6ccccc6ccc54)cc3)CCCC21]

Compound P 228 of 7.99 g (yield 68%) was obtained by synthesis and purification in the same manner as the manufacturing method of Compound P 44 except that Sub 10 (8.00 g, 15.45 mmol) and 4-(4-bromophenyl)dibenzo[b,d]furan (5.50 g, 17.00 mmol) were used.

### II-10 Synthesis of Compound P311

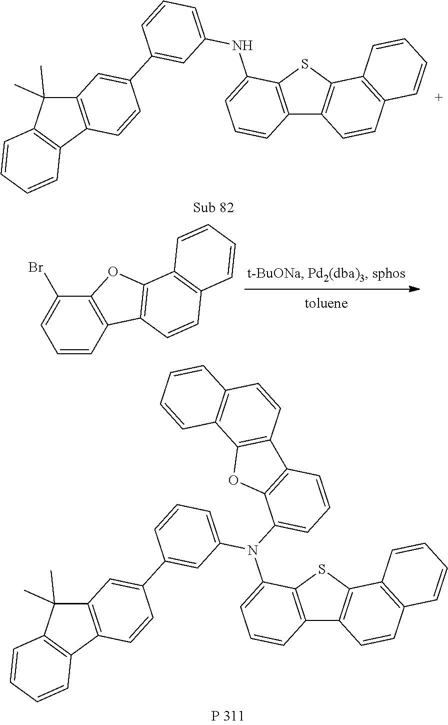

Compound P 311 of 7.49 g (yield 66%) was obtained by synthesis and purification in the same manner as the manufacturing method of Compound P 44 except that Sub 82 (4.79 g, 17.54 mmol) and 10-bromonaphtho[1,2-b]benzofuran (4.79 g, 17.54 mmol) were used.

### II-11 Synthesis of Compound P 640

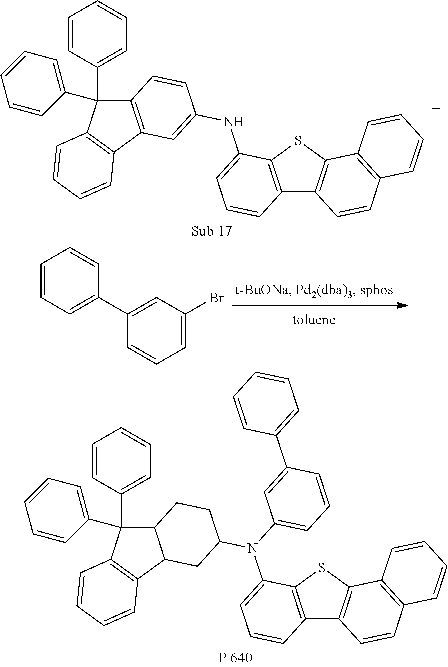 [SMILES: c1ccc(C2(c3ccccc3)c3ccccc3-c3cc(Nc4cccc5c4sc4c6ccccc6ccc54)ccc32)cc1 ; Brc1cccc(-c2ccccc2)c1 ; c1ccc(-c2cccc(N(c3cccc4c3sc3c5ccccc5ccc43)C3CCC4C(C3)c3ccccc3C4(c3ccccc3)c3ccccc3)c2)cc1]

Compound P 640 of 6.90 g (yield 68%) was obtained by synthesis and purification in the same manner as the manufacturing method of Compound P 44 except that Sub 17 (8.00 g, 14.14 mmol) and 3-bromo-1,1′-biphenyl (3.63 g, 15.56 mmol) were used.

Table 2 below shows FD-MS values of some of the final compounds

TABLE 2

| Compound | FD-MS | Compound | FD-MS |
|---|---|---|---|
| P 1 | m/z = 500.71 (C₃₇H₂₇NO = 501.21) | P 2 | m/z = 550.52 (C₄₁H₂₉NO = 551.22) |
| P 5 | m/z = 616.57 (C₄₆H₃₅NO = 617.27) | P 8 | m/z = 590.52 (C₄₃H₂₉NO₂ = 591.22) |
| P 15 | m/z = 576.54 (C₄₃H₃₁NO = 577.24) | P 21 | m/z = 682.53 (C₄₉H₃₃NOS = 683.23) |
| P 29 | m/z = 692.6 (C₅₂H₃₉NO = 693.30) | P 31 | m/z = 692.6 (C₅₂H₃₉NO = 693.30) |
| P 44 | m/z = 652.57 (C₄₉H₃₅NO = 653.27) | P 45 | m/z = 702.59 (C₅₃H₃₇NO = 703.29) |
| P 48 | m/z = 758.56 (C₅₅H₃₇NOS = 759.26) | P 51 | m/z = 741.6 (C₅₅H₃₈N₂O = 742.30) |
| P 59 | m/z = 758.46 (C₅₅H₃₇NOS = 759.26) | P 62 | m/z = 808.58 (C₅₉H₃₉NOS = 809.28) |
| P 71 | m/z = 728.6 (C₅₅H₃₉NO = 729.30) | P 78 | m/z = 699.65 (C₅₂H₃₂D₇NO = 700.35) |
| P 80 | m/z = 681.75 (C₄₉H₆D₂₉NO = 682.45) | P 83 | m/z = 652.57 (C₄₉H₃₅NO = 653.27) |
| P 84 | m/z = 728.6 (C₅₅H₃₉NO = 729.30) | P 89 | m/z = 756.63 (C₅₇H₄₃NO = 757.33) |
| P 93 | m/z = 600.64 (C₄₅H₃₁NO = 601.24) | P 95 | m/z = 616.57 (C₄₆H₃₅NO = 617.27) |
| P 97 | m/z = 590.52 (C₄₃H₂₉NO₂ = 591.22) | P 103 | m/z = 626.66 (C₄₇H₃₃NO = 627.26) |
| P 107 | m/z = 682.63 (C₄₉H₃₃NOS = 683.23) | P 115 | m/z = 692.6 (C₅₂H₃₉NO = 693.30) |
| P 117 | m/z = 666.65 (C₄₉H₃₃NO₂ = 667.25) | P 121 | m/z = 741.7 (C₅₅H₃₈N₂O = 742.30) |
| P 129 | m/z = 698.51 (C₄₉H₃₃NS₂ = 699.21) | P 132 | m/z = 652.57 (C₄₉H₃₅NO = 653.27) |
| P 139 | m/z = 741.6 (C₅₅H₃₈N₂O = 742.30) | P 141 | m/z = 652.67 (C₄₉H₃₅NO = 653.27) |
| P 146 | m/z = 758.66 (C₅₅H₃₇NOS = 759.26) | P 151 | m/z = 652.67 (C₄₉H₃₅NO = 653.27) |
| P 159 | m/z = 758.66 (C₅₅H₃₇NOS = 759.26) | P 162 | m/z = 792.7 (C₅₉H₃₉NO₂ = 793.30) |
| P 169 | m/z = 672.8 (C₄₉H₁₅D₂₀NO = 673.40) | P 170 | m/z = 681.85 (C₄₉H₆D₂₉NO = 682.45) |
| P 175 | m/z = 728.7 (C₅₅H₃₉NO = 729.30) | P 177 | m/z = 658.72 (C₄₉H₄₁NO = 659.32) |
| P 181 | m/z = 516.59 (C₃₇H₂₇NS = 517.19) | P 183 | m/z = 566.6 (C₄₁H₂₉NS = 567.20) |
| P 186 | m/z = 632.65 (C₄₆H₃₅NS = 633.25) | P 189 | m/z = 606.6 (C₄₃H₂₉NOS 607.20) |
| P 195 | m/z = 592.62 (C₄₃H₃₁NS = 593.22) | P 197 | m/z = 692.65 (C₅₁H₃₅NS = 693.25) |
| P 199 | m/z = 682.63 (C₄₉H₃₃NOS = 683.23) | P 203 | m/z = 757.68 (C₅₅H₃₈N₂S = 758.28) |
| P 204 | m/z = 681.64 (C₄₉H₃₄N₂S = 682.24) | P 215 | m/z = 682.63 (C₄₉H₃₃NOS = 683.23) |
| P 221 | m/z = 698.61 (C₄₉H₃₃NS₂ = 699.21) | P 228 | m/z = 758.66 (C₅₅H₃₇NOS = 759.26) |
| P 234 | m/z = 718.66 (C₅₃H₃₇NS = 719.26) | P 237 | m/z = 758.66 (C₅₅H₃₇NOS = 759.26) |
| P 243 | m/z = 668.65 (C₄₉H₃₅NS = 669.25) | P 253 | m/z = 860.74 (C₆₄H₄₇NS = 861.34) |
| P 259 | m/z = 688.77 (C₄₉H₁₅D₂₀NS = 689.37) | P 260 | m/z = 697.83 (C₄₉H₆D₂₉NS = 698.43) |
| P 261 | m/z = 648.68 (C₄₇H₃₉NS = 649.28) | P 268 | m/z = 740.69 (C₅₂H₄₃NSSi = 741.29) |
| P 271 | m/z = 516.59 (C₃₇H₂₇NS = 517.19) | P 272 | m/z = 566.6 (C₄₁H₂₉NS = 567.20) |
| P 278 | m/z = 622.57 (C₄₃H₂₉NS₂ = 623.17) | P 282 | m/z = 592.62 (C₄₃H₃₁NS = 593.22) |
| P 287 | m/z = 698.61 (C₄₉H₃₃NS₂ = 699.21) | P 295 | m/z = 708.58 (C₅₂H₃₉NS = 709.28) |
| P 299 | m/z = 698.61 (C₄₉H₃₃NS₂ = 699.21) | P 302 | m/z = 732.64 (C₅₃H₃₅NOS = 733.24) |
| P 303 | m/z = 642.63 (C₄₇H₃₃NS = 643.23) | P 307 | m/z = 682.63 (C₄₉H₃₃NOS = 683.23) |
| P 311 | m/z = 732.64 (C₅₃H₃₅NOS = 733.24) | P 320 | m/z = 824.65 (C₅₉H₃₉NS₂ = 825.25) |
| P 323 | m/z = 784.71 (C₅₈H₄₃NS = 785.31) | P 328 | m/z = 833.71 (C₆₁H₄₂N₂S = 834.31) |
| P 339 | m/z = 774.64 (C₅₅H₃₇NS₂ = 775.24) | P 343 | m/z = 744.68 (C₅₅H₃₉NS = 745.28) |
| P 348 | m/z = 715.72 (C₅₂H₃₂D₇NS = 716.32) | P 350 | m/z = 697.83 (C₄₉H₆D₂₉NS = 698.43) |
| P 353 | m/z = 668.65 (C₄₉H₃₅NS = 669.25) | P 356 | m/z = 686.64 (C₄₉H₃₄FNS = 687.24) |
| P 361 | m/z = 624.64 (C₄₇H₃₁NO = 625.24) | P 362 | m/z = 674.66 (C₅₁H₃₃NO = 675.26) |
| P 365 | m/z = 740.70 (C₅₆H₃₉NO = 741.30) | P 366 | m/z = 740.70 (C₅₆H₃₉NO = 741.30) |
| P 368 | m/z = 714.65 (C₅₃H₃₃NO₂ = 715.25) | P 371 | m/z = 730.63 (C₅₃H₃₃NOS = 731.23) |
| P 376 | m/z = 700.67 (C₅₃H₃₅NO = 701.27) | P 379 | m/z = 790.68 (C₅₉H₃₇NO₂ = 791.28) |
| P 382 | m/z = 806.66 (C₅₉H₃₇NOS = 807.26) | P 384 | m/z = 789.70 (C₅₉H₃₈N₂O = 790.30) |
| P 387 | m/z = 750.59 (C₅₇H₃₇NO = 751.29) | P 388 | m/z = 816.63 (C₆₂H₄₃NO = 817.33) |
| P 391 | m/z = 790.68 (C₅₉H₃₇NO₂ = 791.28) | P 398 | m/z = 816.63 (C₆₂H₄₃NO = 817.33) |
| P 406 | m/z = 776.60 (C₅₉H₃₉NO = 777.30) | P 416 | m/z = 776.60 (C₅₉H₃₉NO = 777.30) |
| P 424 | m/z = 776.60 (C₅₉H₃₉NO = 777.30) | P 432 | m/z = 852.63 (C₆₅H₄₃NO = 853.33) |
| P 439 | m/z = 796.73 (C₅₉H₁₉D₂₀NO = 797.43) | P 440 | m/z = 735.79 (C₅₃D₃₅NO = 736.49) |
| P 443 | m/z = 776.60 (C₅₉H₃₉NO = 777.30) | P 451 | m/z = 624.64 (C₄₇H₃₁NO = 625.24) |
| P 459 | m/z = 714.65 (C₅₃H₃₃NO₂ = 715.25) | P 461 | m/z = 730.63 (C₅₃H₃₃NOS = 731.23) |
| P 465 | m/z = 700.67 (C₅₃H₃₅NO = 701.27) | P 467 | m/z = 816.63 (C₆₂H₄₃NO = 817.33) |
| P 473 | m/z = 840.60 (C₆₃H₃₉NO₂ = 841.30) | P 479 | m/z = 806.66 (C₅₉H₃₇NOS = 807.26) |
| P 482 | m/z = 700.67 (C₅₃H₃₅NO = 701.27) | P 487 | m/z = 806.66 (C₅₉H₃₇NOS = 807.26) |
| P 494 | m/z = 866.61 (C₆₅H₄₁NO₂ = 867.31) | P 498 | m/z = 776.60 (C₅₉H₃₉NO = 777.30) |
| P 502 | m/z = 882.59 (C₆₆H₄₁NOS = 883.29) | P 506 | m/z = 776.60 (C₅₉H₃₉NO = 777.30) |
| P 515 | m/z = 852.63 (C₆₅H₄₃NO = 853.33) | P 519 | m/z = 958.72 (C₇₁H₄₅NOS = 959.32) |
| P 522 | m/z = 780.73 (C₅₉H₃₅D₄NO = 781.33) | P 523 | m/z = 823.78 (C₆₂H₃₆D₇NO = 824.38) |
| P 529 | m/z = 852.63 (C₆₅H₄₃NO = 853.33) | P 532 | m/z = 782.75 (C₅₉H₄₅NO = 783.35) |
| P 536 | m/z = 640.52 (C₄₇H₃₁NS = 641.22) | P 538 | m/z = 690.53 (C₅₁H₃₃NS = 691.23) |
| P 540 | m/z = 756.58 (C₅₆H₃₉NS = 757.28) | P 543 | m/z = 730.53 (C₅₃H₃₃NOS = 731.23) |
| P 549 | m/z = 780.54 (C₅₇H₃₅NOS = 781.24) | P 551 | m/z = 714.55 (C₅₃H₃₅NS = 717.25) |
| P 554 | m/z = 806.56 (C₅₉H₃₇NOS = 807.26) | P 559 | m/z = 805.58 (C₅₉H₃₈N₂S = 806.28) |
| P 561 | m/z = 716.55 (C₅₃H₃₅NS = 717.25) | P 568 | m/z = 881.61 (C₆₅H₄₂N₂S = 882.31) |
| P 573 | m/z = 832.71 (C₆₂H₄₃NS = 833.31) | P 580 | m/z = 792.68 (C₅₉H₃₉NS = 793.28) |
| P 584 | m/z = 882.69 (C₆₅H₄₁NOS = 883.29) | P 591 | m/z = 792.68 (C₅₉H₃₉NS = 793.28) |
| P 599 | m/z = 792.68 (C₅₉H₃₉NS = 793.28) | P 601 | m/z = 908.74 (C₆₈H₄₇NS = 909.34) |
| P 613 | m/z = 839.76 (C₆₂H₃₆D₇NS = 840.36) | P 615 | m/z = 751.87 (C₅₃D₃₅NS = 752.47) |
| P 616 | m/z = 772.71 (C₅₇H₄₃NS = 773.31) | P 622 | m/z = 798.73 (C₅₉H₄₅NS = 799.33) |
| P 626 | m/z = 640.52 (C₄₇H₃₁NS = 641.22) | P 629 | m/z = 716.55 (C₅₅H₃₅NS = 741.25) |
| P 631 | m/z = 756.58 (C₅₆H₃₉NS = 757.28) | P 640 | m/z = 716.55 (C₅₃H₃₅NS = 717.25) |
| P 641 | m/z = 766.66 (C₅₇H₃₇NS = 767.26) | P 644 | m/z = 822.54 (C₅₉H₃₇NS₂ = 823.24) |
| P 649 | m/z = 716.55 (C₅₃H₃₅NS = 717.25) | P 651 | m/z = 832.71 (C₆₂H₄₃NS = 833.31) |
| P 658 | m/z = 766.66 (C₅₇H₃₇NS = 767.26) | P 662 | m/z = 822.54 (C₅₉H₃₇NS₂ = 823.24) |
| P 668 | m/z = 908.74 (C₆₈H₄₇NS = 909.34) | P 671 | m/z = 881.61 (C₆₅H₄₂N₂S = 882.31) |
| P 676 | m/z = 882.69 (C₆₅H₄₁NOS = 883.29) | P 681 | m/z = 792.68 (C₅₉H₃₉NS = 793.28) |
| P 688 | m/z = 881.61 (C₆₅H₄₂N₂S = 882.31) | P 691 | m/z = 918.63 (C₆₉H₄₅NS = 919.33) |
| P 697 | m/z = 796.61 (C₅₉H₃₅D₄NS = 797.31) | P 698 | m/z = 839.66 (C₆₂H₃₆D₇NS = 840.36) |
| P 703 | m/z = 792.68 (C₅₉H₃₉NS = 793.28) | P 709 | m/z = 896.74 (C₆₇H₄₇NS = 897.34) |

### Example 1: Manufacturing of Organic Light Emitting Diode (Blue Light Emitting Layer)

A positive electrode was formed with ITO on a substrate on which a reflective layer was formed and surface-treated with N₂ plasma or UV-ozone. HAT-CN was deposited above the positive electrode in a thickness of 10 nm as a hole injection layer (HIL). Subsequently, the hole transport layer (HTL) was formed by depositing Compound P 5 in a thickness of 110 nm. A hole transport auxiliary layer was formed above the hole transport layer by forming N4,N4,N4′,N4′-tetra([1,1′-biphenyl]-4-yl)-[1,1′-biphenyl]-4,4′-diamine in a thickness of 15 nm through vacuum deposition, and to form a blue light emitting layer as the light emitting layer (EML) above the hole transport auxiliary layer, N1,N1,N6,N6-tetrakis(4-(1-silyl)phenyl)pyrene-1,6-diamine as a dopant was doped at about 3 wt % while 9,10-Bis(2-naphthyl)anthracene (ADN) as a host was deposited in a thickness of 25 nm. An electron transport layer (ETL) was deposited above the blue light emitting layer in a thickness of 30 nm by mixing anthracene derivative and Liq at a mass ratio of 1:1, and Liq as an electron injection layer (EIL) was deposited above the electron transport layer (ETL) in a thickness of 1 nm. Then, a mixture mixing magnesium and silver (Ag) at 9:1 was deposited in a thickness of 15 nm as a negative electrode, and N4,N4′-bis[4-[bis(3-Methylphenyl)amino]phenyl]-N4,N4′-diphenyl-[1,1′-biphenyl]-4,4′-diamine (DNTPD) as a capping layer was deposited above the negative electrode in a thickness of 60 nm. An organic light emitting diode was manufactured by bonding a seal cap containing a moisture absorbent on the capping layer using a UV curable adhesive and forming a protecting film (encapsulation layer or protecting layer) to protect the organic light emitting diode from oxygen or moisture in the atmosphere.

### Examples 2 to 106

Organic light emitting diodes of Examples 2 to 106 and Comparative Example 1 were manufactured in the same manner as Example 1 except that the material of Compound P 5 used as the material for a hole transport layer in Example 1 was changed to one expressed in Table 3 below. The material for a hole transport layer used in Comparative Example 1 is as follows.

### Material for Hole Transport Layer in Comparative Example 1

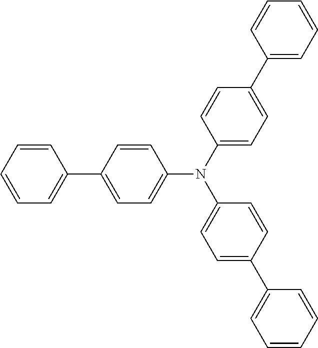 [SMILES: c1ccc(-c2ccc(N(c3ccc(-c4ccccc4)cc3)c3ccc(-c4ccccc4)cc3)cc2)cc1]

### Experimental Example 1: Device Performance Analysis

For the organic light emitting diodes manufactured in Examples 1 to 106 and Comparative Example 1, operation voltage (unit: V) and efficiency, which are light-emitting characteristics when driven at a current of 10 mA/cm², and lifetime (T95, unit: hrs) reduced by 95% when driven at a constant current of 20 mA/cm² were measured, and thus a result of measurement is expressed in Table 3 below.

TABLE 3

| | Material | | Efficiency | | | | | |
|---|---|---|---|---|---|---|---|---|
| | for Hole | Operation | | | EQE | Color | | Lifetime |
| Example | Transport Layer | Voltage | Cd/A | Im/A | (%) | CIEx | CIEy | T95 |
| 1 | P 5 | 3.50 | 8.2 | 7.4 | 17.9 | 0.139 | 0.044 | 340 |
| 2 | P 7 | 3.54 | 7.8 | 6.9 | 17.0 | 0.139 | 0.043 | 325 |
| 3 | P 10 | 3.59 | 8.7 | 7.6 | 19.5 | 0.140 | 0.041 | 313 |
| 4 | P 26 | 3.67 | 9.2 | 7.8 | 18.3 | 0.136 | 0.049 | 305 |
| 5 | P 29 | 3.62 | 9.0 | 7.8 | 17.9 | 0.136 | 0.050 | 346 |
| 6 | P 31 | 3.59 | 8.7 | 7.6 | 17.8 | 0.137 | 0.048 | 360 |
| 7 | P 36 | 3.65 | 8.6 | 7.4 | 17.5 | 0.137 | 0.048 | 340 |
| 8 | P 38 | 3.53 | 8.8 | 7.9 | 17.9 | 0.137 | 0.048 | 350 |
| 9 | P 1065 | 3.52 | 9.0 | 8.0 | 18.4 | 0.137 | 0.047 | 320 |
| 10 | P 44 | 3.54 | 8.8 | 7.8 | 17.8 | 0.137 | 0.048 | 360 |
| 11 | P 45 | 3.51 | 9.7 | 8.7 | 18.4 | 0.134 | 0.054 | 312 |
| 12 | P 50 | 3.59 | 10.0 | 8.8 | 18.5 | 0.134 | 0.055 | 305 |
| 13 | P 51 | 3.53 | 8.0 | 7.1 | 17.1 | 0.138 | 0.045 | 390 |
| 14 | P 61 | 3.60 | 8.0 | 7.0 | 16.9 | 0.138 | 0.046 | 380 |
| 15 | P 79 | 3.47 | 7.7 | 7.0 | 16.4 | 0.138 | 0.045 | 415 |
| 16 | P 80 | 3.51 | 8.1 | 7.3 | 17.5 | 0.139 | 0.044 | 430 |
| 17 | P 81 | 3.45 | 8.0 | 7.3 | 17.2 | 0.139 | 0.044 | 346 |
| 18 | P 86 | 3.44 | 8.0 | 7.3 | 17.3 | 0.139 | 0.044 | 333 |
| 19 | P 87 | 3.46 | 8.2 | 7.4 | 18.0 | 0.140 | 0.043 | 366 |
| 20 | P 88 | 3.46 | 8.0 | 7.2 | 17.6 | 0.140 | 0.043 | 359 |
| 21 | P 97 | 3.48 | 7.3 | 6.6 | 15.5 | 0.138 | 0.045 | 327 |
| 22 | P 103 | 3.54 | 8.3 | 7.3 | 16.8 | 0.137 | 0.048 | 338 |
| 23 | P 121 | 3.48 | 8.9 | 8.0 | 18.0 | 0.137 | 0.048 | 325 |
| 24 | P 126 | 3.53 | 8.9 | 7.9 | 18.2 | 0.137 | 0.048 | 377 |
| 25 | P 128 | 3.54 | 8.8 | 7.8 | 18.2 | 0.137 | 0.047 | 352 |
| 26 | P 129 | 3.72 | 9.0 | 7.6 | 18.4 | 0.137 | 0.047 | 360 |
| 27 | P 132 | 3.59 | 8.5 | 7.4 | 17.6 | 0.138 | 0.047 | 365 |
| 28 | P 138 | 3.59 | 9.3 | 8.2 | 18.7 | 0.137 | 0.049 | 343 |
| 29 | P 140 | 3.58 | 9.2 | 8.1 | 18.2 | 0.136 | 0.050 | 321 |
| 30 | P 141 | 3.50 | 9.1 | 8.1 | 17.9 | 0.136 | 0.050 | 340 |
| 31 | P 145 | 3.48 | 9.0 | 8.2 | 17.8 | 0.136 | 0.051 | 350 |
| 32 | P 147 | 3.46 | 7.4 | 6.8 | 16.0 | 0.139 | 0.045 | 393 |
| 33 | P 150 | 3.56 | 8.9 | 7.9 | 17.9 | 0.136 | 0.049 | 385 |
| 34 | P 156 | 3.55 | 9.1 | 8.1 | 18.0 | 0.136 | 0.050 | 371 |
| 35 | P 163 | 3.49 | 8.8 | 7.9 | 17.9 | 0.137 | 0.048 | 330 |
| 36 | P 168 | 3.46 | 8.2 | 7.5 | 17.2 | 0.138 | 0.046 | 440 |
| 37 | P 169 | 3.56 | 9.3 | 8.2 | 18.5 | 0.136 | 0.050 | 490 |
| 38 | P 170 | 3.61 | 9.1 | 7.9 | 18.1 | 0.137 | 0.049 | 455 |
| 39 | P 171 | 3.55 | 9.2 | 8.1 | 18.0 | 0.136 | 0.051 | 345 |
| 40 | P 174 | 3.46 | 8.0 | 7.2 | 16.8 | 0.138 | 0.045 | 385 |
| 41 | P 177 | 3.56 | 8.2 | 7.3 | 17.2 | 0.138 | 0.046 | 383 |
| 42 | P 181 | 3.60 | 8.7 | 7.6 | 18.3 | 0.138 | 0.046 | 356 |
| 43 | P 185 | 3.57 | 8.5 | 7.4 | 17.8 | 0.138 | 0.046 | 325 |
| 44 | P 191 | 3.59 | 8.6 | 7.6 | 17.9 | 0.137 | 0.047 | 395 |
| 45 | P 195 | 3.60 | 7.3 | 6.4 | 14.9 | 0.137 | 0.048 | 350 |
| 46 | P 202 | 3.64 | 9.0 | 7.8 | 18.3 | 0.137 | 0.048 | 360 |
| 47 | P 204 | 3.60 | 9.1 | 7.9 | 18.4 | 0.136 | 0.049 | 375 |
| 48 | P 206 | 3.59 | 9.3 | 8.1 | 18.8 | 0.136 | 0.049 | 380 |
| 49 | P 215 | 3.49 | 9.1 | 8.2 | 18.4 | 0.136 | 0.049 | 355 |
| 50 | P 219 | 3.65 | 9.1 | 7.8 | 18.5 | 0.136 | 0.048 | 335 |
| 51 | P 222 | 3.67 | 8.4 | 7.2 | 17.4 | 0.138 | 0.047 | 350 |
| 52 | P 223 | 3.61 | 9.1 | 7.9 | 18.3 | 0.136 | 0.049 | 390 |
| 53 | P 227 | 3.49 | 9.1 | 8.2 | 18.3 | 0.136 | 0.049 | 350 |
| 54 | P 229 | 3.52 | 9.2 | 8.2 | 18.6 | 0.137 | 0.048 | 350 |
| 55 | P 240 | 3.51 | 9.1 | 8.1 | 18.2 | 0.136 | 0.049 | 361 |
| 56 | P 241 | 3.56 | 8.4 | 7.4 | 17.6 | 0.138 | 0.046 | 320 |
| 57 | P 242 | 3.67 | 8.3 | 7.1 | 17.5 | 0.138 | 0.046 | 320 |
| 58 | P 253 | 3.56 | 8.7 | 7.6 | 17.8 | 0.137 | 0.047 | 287 |
| 59 | P 258 | 3.56 | 8.8 | 7.8 | 18.1 | 0.137 | 0.047 | 405 |
| 60 | P 259 | 3.54 | 9.0 | 8.0 | 18.1 | 0.137 | 0.049 | 425 |
| 61 | P 260 | 3.50 | 8.1 | 7.3 | 17.0 | 0.138 | 0.046 | 415 |
| 62 | P 270 | 3.52 | 9.2 | 8.2 | 17.7 | 0.135 | 0.052 | 300 |
| 63 | P 282 | 3.58 | 9.5 | 8.3 | 18.3 | 0.135 | 0.052 | 318 |
| 64 | P 301 | 3.61 | 9.6 | 8.3 | 18.2 | 0.134 | 0.053 | 305 |
| 65 | P 302 | 3.56 | 8.4 | 7.4 | 17.8 | 0.138 | 0.046 | 315 |
| 66 | P 307 | 3.56 | 8.5 | 7.5 | 17.9 | 0.138 | 0.046 | 312 |
| 67 | P 320 | 3.61 | 7.7 | 6.7 | 16.8 | 0.139 | 0.043 | 336 |
| 68 | P 328 | 3.58 | 9.6 | 8.4 | 18.1 | 0.135 | 0.053 | 440 |
| 69 | P 330 | 3.57 | 9.7 | 8.5 | 17.9 | 0.134 | 0.055 | 410 |
| 70 | P 345 | 3.54 | 7.9 | 7.0 | 16.5 | 0.138 | 0.046 | 370 |
| 71 | P 353 | 3.48 | 8.1 | 7.3 | 17.4 | 0.139 | 0.044 | 320 |
| 72 | P 349 | 3.47 | 7.9 | 7.2 | 16.9 | 0.138 | 0.045 | 435 |
| 73 | P 350 | 3.47 | 8.2 | 7.4 | 17.2 | 0.138 | 0.046 | 410 |
| 74 | P 365 | 3.42 | 7.7 | 7.0 | 18.8 | 0.143 | 0.037 | 325 |
| 75 | P 370 | 3.44 | 7.1 | 6.5 | 15.6 | 0.140 | 0.043 | 380 |
| 76 | P 374 | 3.56 | 8.0 | 7.1 | 17.2 | 0.139 | 0.044 | 298 |
| 77 | P 375 | 3.49 | 7.9 | 7.1 | 17.1 | 0.139 | 0.044 | 285 |
| 78 | P 388 | 3.55 | 8.9 | 7.9 | 17.9 | 0.136 | 0.049 | 360 |
| 79 | P 394 | 3.65 | 9.1 | 7.8 | 18.1 | 0.137 | 0.049 | 380 |
| 80 | P 401 | 3.55 | 9.3 | 8.2 | 17.6 | 0.134 | 0.053 | 296 |
| 81 | P 402 | 3.54 | 8.8 | 7.8 | 17.7 | 0.137 | 0.049 | 310 |
| 82 | P 423 | 3.53 | 8.5 | 7.5 | 17.4 | 0.137 | 0.047 | 340 |
| 83 | P 432 | 3.55 | 10.5 | 9.3 | 17.8 | 0.129 | 0.064 | 354 |
| 84 | P 440 | 3.49 | 8.1 | 7.3 | 17.2 | 0.139 | 0.045 | 415 |
| 85 | P 449 | 3.53 | 9.9 | 8.8 | 19.0 | 0.134 | 0.053 | 341 |
| 86 | P 465 | 3.53 | 9.3 | 8.3 | 18.8 | 0.136 | 0.049 | 344 |
| 87 | P 480 | 3.53 | 9.9 | 8.8 | 17.8 | 0.133 | 0.057 | 356 |
| 88 | P 497 | 3.72 | 10.1 | 8.5 | 17.8 | 0.132 | 0.059 | 442 |
| 89 | P 523 | 3.50 | 9.7 | 8.7 | 17.8 | 0.134 | 0.056 | 368 |
| 90 | P 540 | 3.53 | 9.9 | 8.8 | 17.8 | 0.133 | 0.057 | 356 |
| 91 | P 568 | 3.65 | 8.0 | 6.9 | 17.1 | 0.139 | 0.045 | 315 |
| 92 | P 626 | 3.54 | 8.1 | 7.2 | 17.2 | 0.139 | 0.045 | 340 |
| 93 | P 614 | 3.87 | 8.9 | 7.2 | 17.5 | 0.135 | 0.051 | 447 |
| 94 | P 615 | 3.88 | 9.0 | 7.3 | 17.6 | 0.136 | 0.051 | 413 |
| 95 | P 644 | 3.77 | 9.6 | 8.0 | 18.0 | 0.134 | 0.054 | 285 |
| 96 | P 651 | 3.75 | 9.8 | 8.2 | 18.6 | 0.134 | 0.053 | 325 |
| 97 | P 700 | 3.70 | 8.8 | 7.5 | 17.8 | 0.136 | 0.048 | 330 |
| 98 | P 703 | 3.70 | 9.0 | 7.6 | 17.9 | 0.136 | 0.050 | 289 |
| 99 | P 741 | 3.65 | 7.7 | 6.6 | 15.8 | 0.139 | 0.046 | 325 |
| 100 | P 33 | 3.68 | 7.6 | 6.5 | 16.1 | 0.139 | 0.046 | 320 |
| 101 | P 34 | 3.61 | 8.3 | 7.2 | 17.0 | 0.137 | 0.048 | 310 |
| 102 | P 800 | 3.60 | 8.3 | 7.2 | 17.0 | 0.137 | 0.047 | 315 |
| 103 | P 84 | 3.48 | 8.4 | 7.5 | 16.8 | 0.137 | 0.049 | 305 |
| 104 | P 47 | 3.61 | 6.8 | 5.9 | 14.7 | 0.140 | 0.043 | 330 |
| 105 | P 733 | 3.63 | 7.4 | 6.4 | 16.0 | 0.139 | 0.044 | 350 |
| 106 | P 1091 | 3.48 | 8.8 | 7.9 | 17.1 | 0.136 | 0.051 | 350 |
| Comparative | Comparative | 3.84 | 4.3 | 3.5 | 9.2 | 0.143 | 0.044 | 140 |
| Example 1 | Example | | | | | | | |
| | Compound | | | | | | | |

### Example 107: Manufacturing of Organic Light Emitting Diode (Green Light Emitting Layer)

A positive electrode was formed with ITO on a substrate on which a reflective layer was formed and surface-treated with N₂ plasma or UV-ozone. HAT-CN was deposited above the positive electrode in a thickness of 10 nm as a hole injection layer (HIL). Subsequently, a hole transport layer (HTL) was formed by depositing N4,N4,N4′,N4′-tetra([1,1′-biphenyl]-4-yl)-[1,1′-biphenyl]-4,4′-diamine in a thickness of 110 nm. A hole transport auxiliary layer was formed by forming Compound P 5 the present invention above the hole transport layer by vacuum deposition in a thickness of 40 nm, and to form a green light emitting layer (EML) above the hole transport auxiliary layer, Ir(ppy)₃[tris(2-phenylpyridine)-iridium] as a dopant was doped at about 5 wt % while 4,4′-N,N′-dicarbazole-biphenyl(CBP) as a host was deposited in a thickness of 35 nm. An electron transport layer (ETL) was deposited above the green light emitting layer in a thickness of 30 nm by mixing anthracene derivative and Liq at a mass ratio of 1:1, and Liq as an electron injection layer (EIL) was deposited above the electron transport layer (ETL) in a thickness of 1 nm. Then, a mixture mixing magnesium and silver (Ag) at 1:4 was deposited in a thickness of 16 nm as a negative electrode, and N4,N4′-bis[4-[bis(3-Methylphenyl)amino]phenyl]-N4,N4′-diphenyl-[1,1′-biphenyl]-4,4′-diamine (DNTPD) as a capping layer was deposited above the negative electrode in a thickness of 60 nm. An organic light emitting diode was manufactured by bonding a seal cap containing a moisture absorbent on the capping layer using a UV curable adhesive and forming a protecting film (encapsulation layer or protecting layer) to protect the organic light emitting diode from oxygen or moisture in the atmosphere.

### Examples 108 to 212

Organic light emitting diodes of Examples 108 to 212 and Comparative Example 2 were each manufactured in the same manner as Example 107 except that the material of Compound P 5 used as the material for a hole transport auxiliary layer in Example 107 was changed to one expressed in Table 4 below. The material for a hole transport auxiliary layer used in Comparative Example 2 is as follows.

### Material for Hole Transport Auxiliary Layer in Comparative Example 2

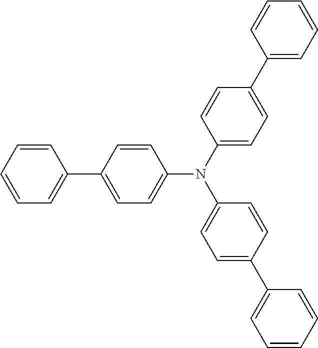 [SMILES: c1ccc(-c2ccc(N(c3ccc(-c4ccccc4)cc3)c3ccc(-c4ccccc4)cc3)cc2)cc1]

### Experimental Example 2: Device Performance Analysis

For the organic light emitting diodes manufactured in Examples 107 to 212 and Comparative Example 2, operation voltage (unit: V) and efficiency, which are light-emitting characteristics when driven at a current of 10 mA/cm² and lifetime (T95, unit: hrs) reduced by 95% when driven at a constant current of 20 mA/cm² were measured, and thus a result of measurement is expressed in Table 4 below.

TABLE 4

| | Material | | | | | | | |
|---|---|---|---|---|---|---|---|---|
| | for Hole | | Efficiency | | | | | |
| | Transport | Operation | | | EQE | Color | | Lifetime |
| Example | Auxiliary Layer | Voltage | Cd/A | Im/A | (%) | CIEx | CIEy | T95 |
| 107 | P 5 | 3.77 | 125.9 | 104.9 | 30.4 | 0.232 | 0.722 | 280 |
| 108 | P 7 | 3.76 | 128.0 | 106.7 | 31.0 | 0.233 | 0.722 | 235 |
| 109 | P 10 | 3.88 | 130.2 | 105.3 | 31.6 | 0.231 | 0.723 | 250 |
| 110 | P 26 | 3.73 | 136.8 | 115.2 | 31.6 | 0.267 | 0.703 | 245 |
| 111 | P 29 | 3.91 | 152.7 | 122.7 | 34.9 | 0.267 | 0.706 | 255 |
| 112 | P 31 | 3.92 | 159.6 | 127.9 | 36.6 | 0.260 | 0.711 | 260 |
| 113 | P 36 | 3.94 | 157.6 | 125.5 | 36.2 | 0.249 | 0.720 | 230 |
| 114 | P 38 | 3.94 | 158.1 | 126.2 | 36.3 | 0.249 | 0.720 | 255 |
| 115 | P 1065 | 3.77 | 124.4 | 103.6 | 30.2 | 0.224 | 0.728 | 275 |
| 116 | P 44 | 3.80 | 129.0 | 106.6 | 31.3 | 0.227 | 0.726 | 240 |
| 117 | P 45 | 4.06 | 151.8 | 117.5 | 34.5 | 0.274 | 0.701 | 220 |
| 118 | P 50 | 3.76 | 157.1 | 131.1 | 35.8 | 0.270 | 0.703 | 240 |
| 119 | P 51 | 3.99 | 159.8 | 126.0 | 36.7 | 0.249 | 0.720 | 285 |
| 120 | P 61 | 3.76 | 126.5 | 101.6 | 30.8 | 0.222 | 0.729 | 270 |
| 121 | P 79 | 3.86 | 144.2 | 117.4 | 33.7 | 0.272 | 0.697 | 330 |
| 122 | P 80 | 3.83 | 145.6 | 119.5 | 33.8 | 0.286 | 0.687 | 340 |
| 123 | P 81 | 3.59 | 117.0 | 102.4 | 28.6 | 0.227 | 0.723 | 265 |
| 124 | P 86 | 3.87 | 126.9 | 102.9 | 29.6 | 0.275 | 0.695 | 260 |
| 125 | P 87 | 3.77 | 131.2 | 109.5 | 30.6 | 0.274 | 0.696 | 255 |
| 126 | P 88 | 3.83 | 120.0 | 103.6 | 28.1 | 0.268 | 0.700 | 260 |
| 127 | P 97 | 3.90 | 147.1 | 118.5 | 34.8 | 0.250 | 0.713 | 255 |
| 128 | P 103 | 3.90 | 147.1 | 118.5 | 34.8 | 0.250 | 0.713 | 245 |
| 129 | P 121 | 3.92 | 144.9 | 116.1 | 34.3 | 0.245 | 0.717 | 235 |
| 130 | P 126 | 3.88 | 137.5 | 111.4 | 32.5 | 0.246 | 0.716 | 255 |
| 131 | P 128 | 3.82 | 137.8 | 113.2 | 32.6 | 0.244 | 0.717 | 260 |
| 132 | P 129 | 3.86 | 139.5 | 113.5 | 32.7 | 0.254 | 0.712 | 245 |
| 133 | P 132 | 3.84 | 136.8 | 112.0 | 32.2 | 0.251 | 0.713 | 250 |
| 134 | P 138 | 3.86 | 133.5 | 108.8 | 31.9 | 0.247 | 0.713 | 270 |
| 135 | P 140 | 3.93 | 134.1 | 107.3 | 32.1 | 0.238 | 0.720 | 235 |
| 136 | P 141 | 3.85 | 138.6 | 113.1 | 32.5 | 0.254 | 0.711 | 265 |
| 137 | P 145 | 3.92 | 121.2 | 104.2 | 29.5 | 0.238 | 0.715 | 250 |
| 138 | P 147 | 3.89 | 112.3 | 90.8 | 27.8 | 0.226 | 0.722 | 238 |
| 139 | P 150 | 3.90 | 143.7 | 115.8 | 33.7 | 0.260 | 0.707 | 280 |
| 140 | P 156 | 3.99 | 137.6 | 108.2 | 32.5 | 0.263 | 0.703 | 225 |
| 141 | P 163 | 3.86 | 133.5 | 108.8 | 31.9 | 0.247 | 0.713 | 250 |
| 142 | P 168 | 3.86 | 130.8 | 106.5 | 31.4 | 0.242 | 0.716 | 340 |
| 143 | P 169 | 3.84 | 141.3 | 115.6 | 33.3 | 0.259 | 0.707 | 330 |
| 144 | P 170 | 3.91 | 135.5 | 108.8 | 32.2 | 0.242 | 0.718 | 350 |
| 145 | P 171 | 3.82 | 134.1 | 110.3 | 32.1 | 0.243 | 0.716 | 277 |
| 146 | P 174 | 3.84 | 141.3 | 115.6 | 33.3 | 0.259 | 0.707 | 220 |
| 147 | P 177 | 3.99 | 145.2 | 114.2 | 34.3 | 0.256 | 0.709 | 220 |
| 148 | P 181 | 3.89 | 131.6 | 106.4 | 31.8 | 0.228 | 0.726 | 265 |
| 149 | P 185 | 3.85 | 124.3 | 101.4 | 30.5 | 0.224 | 0.726 | 255 |
| 150 | P 191 | 3.95 | 122.3 | 97.2 | 29.9 | 0.227 | 0.723 | 233 |
| 151 | P 195 | 3.85 | 123.3 | 100.7 | 30.2 | 0.225 | 0.725 | 247 |
| 152 | P 202 | 3.80 | 133.6 | 110.3 | 31.7 | 0.242 | 0.719 | 285 |
| 153 | P 204 | 3.89 | 120.5 | 97.3 | 29.3 | 0.247 | 0.709 | 236 |
| 154 | P 206 | 3.72 | 108.2 | 91.4 | 26.8 | 0.235 | 0.714 | 247 |
| 155 | P 215 | 3.68 | 111.2 | 95.0 | 27.4 | 0.237 | 0.713 | 260 |
| 156 | P 219 | 3.59 | 117.0 | 102.4 | 28.6 | 0.227 | 0.723 | 225 |
| 157 | P 222 | 3.62 | 115.6 | 100.4 | 28.3 | 0.227 | 0.724 | 230 |
| 158 | P 223 | 3.64 | 114.3 | 98.5 | 28.1 | 0.227 | 0.722 | 240 |
| 159 | P 227 | 3.88 | 114.5 | 92.8 | 28.0 | 0.234 | 0.718 | 234 |
| 160 | P 229 | 3.98 | 112.4 | 88.8 | 27.6 | 0.231 | 0.719 | 220 |
| 161 | P 240 | 3.42 | 118.8 | 109.1 | 28.1 | 0.256 | 0.707 | 250 |
| 162 | P 241 | 3.42 | 114.4 | 105.0 | 27.5 | 0.237 | 0.719 | 250 |
| 163 | P 242 | 3.45 | 118.0 | 107.6 | 28.4 | 0.239 | 0.718 | 230 |
| 164 | P 253 | 3.49 | 130.9 | 117.9 | 30.9 | 0.258 | 0.706 | 250 |
| 165 | P 258 | 3.80 | 132.0 | 109.1 | 31.4 | 0.283 | 0.685 | 327 |
| 166 | P 259 | 3.78 | 128.9 | 107.1 | 31.2 | 0.242 | 0.713 | 314 |
| 167 | P 260 | 3.84 | 135.4 | 110.7 | 32.2 | 0.285 | 0.683 | 338 |
| 168 | P 270 | 4.06 | 146.9 | 113.8 | 34.1 | 0.280 | 0.692 | 260 |
| 169 | P 282 | 4.00 | 137.2 | 107.7 | 32.5 | 0.271 | 0.696 | 223 |
| 170 | P 301 | 3.81 | 122.5 | 100.9 | 29.9 | 0.235 | 0.718 | 234 |
| 171 | P 302 | 3.78 | 128.9 | 107.1 | 31.2 | 0.242 | 0.713 | 241 |
| 172 | P 307 | 3.69 | 120.9 | 102.8 | 29.4 | 0.242 | 0.713 | 229 |
| 173 | P 320 | 3.70 | 134.7 | 114.5 | 32.2 | 0.262 | 0.701 | 253 |
| 174 | P 328 | 3.62 | 135.3 | 117.3 | 32.3 | 0.263 | 0.700 | 241 |
| 175 | P 330 | 3.79 | 127.5 | 105.6 | 30.4 | 0.257 | 0.705 | 237 |
| 176 | P 345 | 3.78 | 120.5 | 100.2 | 29.2 | 0.252 | 0.706 | 249 |
| 177 | P 353 | 3.77 | 125.8 | 105.0 | 30.6 | 0.236 | 0.718 | 280 |
| 178 | P 349 | 3.63 | 117.1 | 101.2 | 28.7 | 0.237 | 0.715 | 327 |
| 179 | P 350 | 3.79 | 139.5 | 115.7 | 33.4 | 0.251 | 0.709 | 340 |
| 180 | P 365 | 3.84 | 139.0 | 113.6 | 33.2 | 0.245 | 0.714 | 252 |
| 181 | P 370 | 3.87 | 135.2 | 109.8 | 32.7 | 0.244 | 0.712 | 240 |
| 182 | P 374 | 3.93 | 143.3 | 114.5 | 34.1 | 0.249 | 0.712 | 225 |
| 183 | P 375 | 3.91 | 135.7 | 109.1 | 32.7 | 0.245 | 0.713 | 220 |
| 184 | P 388 | 3.75 | 136.2 | 114.0 | 32.6 | 0.239 | 0.719 | 220 |
| 185 | P 394 | 3.78 | 141.9 | 117.9 | 33.5 | 0.253 | 0.711 | 245 |
| 186 | P 401 | 3.74 | 136.3 | 112.5 | 32.1 | 0.263 | 0.703 | 242 |
| 187 | P 402 | 3.56 | 136.5 | 120.4 | 32.2 | 0.252 | 0.712 | 210 |
| 188 | P 423 | 3.68 | 143.1 | 122.1 | 33.5 | 0.265 | 0.702 | 231 |
| 189 | P 432 | 3.79 | 131.0 | 110.0 | 31.8 | 0.257 | 0.705 | 265 |
| 190 | P 440 | 3.86 | 126.9 | 103.3 | 30.8 | 0.225 | 0.728 | 230 |
| 191 | P 449 | 3.85 | 126.8 | 103.4 | 30.7 | 0.226 | 0.728 | 220 |
| 192 | P 465 | 3.95 | 128.6 | 102.3 | 31.1 | 0.226 | 0.728 | 220 |
| 193 | P 480 | 3.87 | 132.6 | 107.9 | 32.3 | 0.233 | 0.721 | 215 |
| 194 | P 497 | 3.82 | 137.1 | 108.2 | 33.1 | 0.229 | 0.725 | 230 |
| 195 | P 523 | 3.78 | 131.1 | 108.9 | 31.7 | 0.224 | 0.729 | 317 |
| 196 | P 540 | 3.89 | 141.4 | 114.2 | 33.6 | 0.246 | 0.716 | 246 |
| 197 | P 568 | 3.78 | 128.9 | 91.6 | 31.2 | 0.212 | 0.716 | 238 |
| 198 | P 626 | 3.77 | 131.8 | 109.8 | 31.8 | 0.229 | 0.726 | 250 |
| 199 | P 614 | 3.79 | 131.0 | 108.6 | 31.6 | 0.30 | 0.726 | 325 |
| 200 | P 615 | 3.84 | 139.0 | 113.6 | 33.2 | 0.245 | 0.714 | 335 |
| 201 | P 644 | 3.86 | 123.0 | 100.0 | 30.2 | 0.219 | 0.730 | 225 |
| 202 | P 651 | 3.88 | 131.3 | 106.3 | 31.5 | 0.238 | 0.720 | 246 |
| 203 | P 700 | 3.89 | 121.3 | 98.1 | 29.8 | 0.219 | 0.730 | 332 |
| 204 | P 703 | 3.86 | 127.5 | 103.4 | 30.8 | 0.232 | 0.723 | 234 |
| 205 | P 741 | 3.83 | 148.1 | 121.4 | 34.7 | 0.261 | 0.706 | 246 |
| 206 | P 33 | 3.85 | 129.8 | 105.8 | 31.3 | 0.233 | 0.723 | 257 |
| 207 | P 34 | 3.87 | 142.7 | 115.8 | 33.4 | 0.264 | 0.704 | 246 |
| 208 | P 800 | 3.86 | 124.2 | 101.0 | 30.1 | 0.232 | 0.723 | 265 |
| 209 | P 84 | 3.88 | 141.3 | 114.5 | 33.6 | 0.248 | 0.714 | 280 |
| 210 | P 47 | 3.77 | 117.6 | 97.9 | 28.5 | 0.232 | 0.722 | 240 |
| 211 | P 733 | 3.87 | 142.1 | 115.4 | 33.7 | 0.248 | 0.714 | 222 |
| 212 | P 1091 | 3.85 | 144.3 | 117.7 | 34.2 | 0.248 | 0.714 | 235 |
| Comparative | Comparative | 4.25 | 93.7 | 69.2 | 23.5 | 0.217 | 0.726 | 165 |
| Example 2 | Example | | | | | | | |
| | Compound | | | | | | | |

Although the embodiments of the specification have been described in more detail, the specification is not necessarily limited to these embodiments, and various modifications can be made without departing from the technical spirit of the specification. Therefore, the embodiments disclosed in the present specification are not intended to limit the technical spirit of the present specification, but for describing it, and the scope of the technical spirit of the present specification is not limited by these embodiments. Therefore, it should be understood that the above-described embodiments are illustrative and not restrictive in all respects.

1. An organic compound represented by Chemical Formula 1 below: 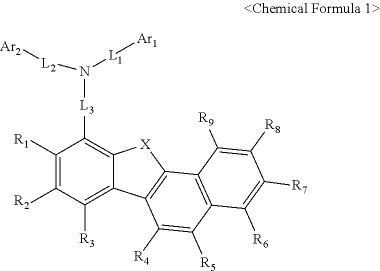
in Chemical Formula 1,
Ar₁ is any one of Chemical Formulas 2, 3, 4, and 5, and
X is O or S, 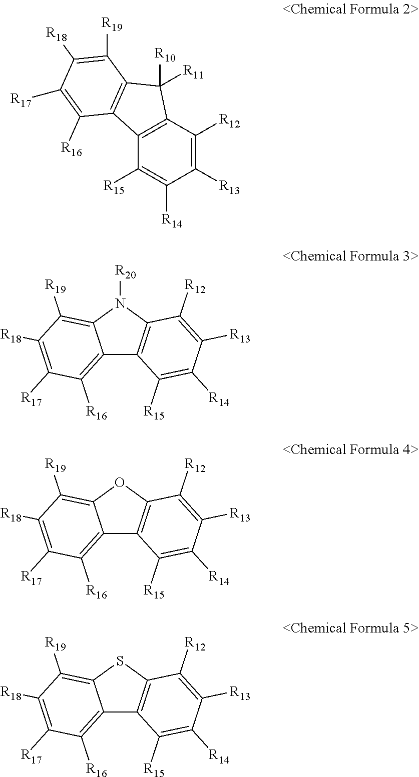
any one of R₁₅ to R₁₉ of the above Chemical Formulas 2 to 5 is bonded to L₁,
L₁ to L₃ are the same as or different from each other and are each independently one selected from the group consisting of a single bond, a substituted or unsubstituted aryl group having 6 to 30 carbon atoms, a substituted or unsubstituted aralkyl group having 7 to 30 carbon atoms, a substituted or unsubstituted heteroaryl group having 5 to 60 carbon atoms, and a substituted or unsubstituted heteroarylalkyl group having 6 to 60 carbon,
R₁ to R₂₀ and Ar₂ are the same as or different from each other and are each independently one selected from the group consisting of hydrogen, deuterium, a cyano group, a nitro group, a halogen, a hydroxy group, a substituted or unsubstituted alkyl group having 1 to 30 carbon atoms, a substituted or unsubstituted alkenyl group having 2 to 30 carbon atoms, a substituted or unsubstituted alkynyl group having 2 to 24 carbon atoms, a substituted or unsubstituted heteroalkyl group having 2 to 30 carbon atoms, a substituted or unsubstituted aryl group having 6 to 30 carbon atoms, a substituted or unsubstituted heteroaryl group having 2 to 30 carbon atoms, a substituted or unsubstituted heteroarylalkyl group having 3 to 30 carbon atoms, a substituted or unsubstituted aralkyl group having 6 to 30 carbon atoms, a substituted or unsubstituted cycloalkyl group having 3 to 20 carbon atoms, a substituted or unsubstituted heterocycloalkyl group having 3 to 20 carbon atoms, a substituted or unsubstituted alkoxy group having 1 to 30 carbon atoms, a substituted or unsubstituted alkylsilyl group having 1 to 30 carbon atoms, a substituted or unsubstituted arylsilyl group having 6 to 30 carbon atoms, and a substituted or unsubstituted aryloxy group having 6 to 30 carbon atoms, two adjacent groups among R₁ to R₂₀ are bonded to form a substituted or unsubstituted carbon ring structure, and
optionally, when L₁ to L₃, R₁ to R₂₀, and Ar₂ are substituted, substituent groups are each independently one or more selected from the group consisting of deuterium, a cyano group, a nitro group, halogen, a hydroxy group, an alkyl group having 1 to 30 carbon atoms, an alkenyl group having 2 to 30 carbon atoms, an alkynyl group having 2 to 24 carbon atoms, a heteroalkyl group having 2 to 30 carbon atoms, an aralkyl group having 6 to 30 carbon atoms, a cycloalkyl group having 3 to 20 carbon atoms, a heterocycloalkyl group having 3 to 20 carbon atoms, an aryl group having 6 to 30 carbon atoms, a heteroaryl group having 2 to 30 carbon atoms, a heteroarylalkyl group having 3 to 30 carbon atoms, an alkoxy group having 1 to 30 carbon atoms, an alkylsilyl group having 1 to 30 carbon atoms, an arylsilyl group having 6 to 30 carbon atoms, and an aryloxy group having 6 to 30 carbon atoms.

2. The organic compound of claim 1, wherein Chemical Formula 1 is one represented by any one of Chemical Formulas 6 to 12 below:

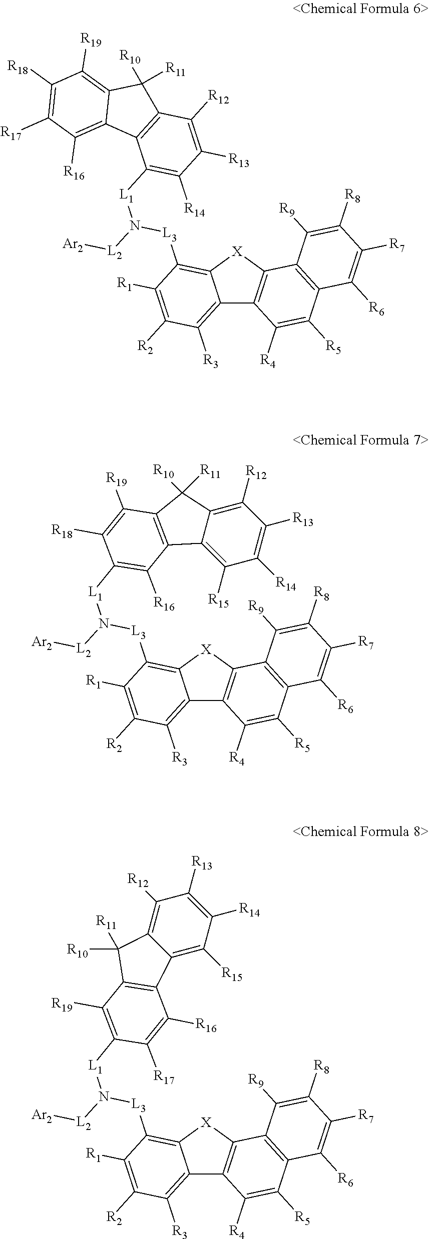

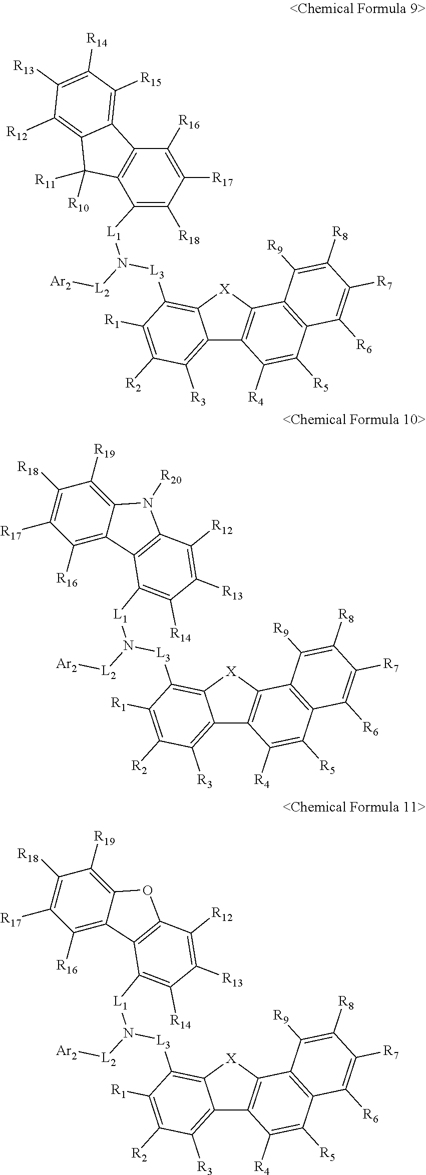

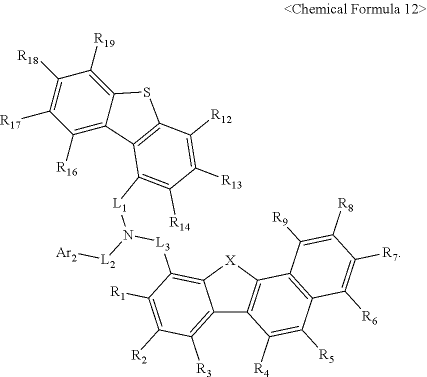

3. The organic compound of claim 1,
wherein Ar₂ is one selected from the group consisting of a substituted or unsubstituted aryl group having 6 to 30 carbon atoms, a substituted or unsubstituted heteroaryl group having 2 to 30 carbon atoms, a substituted or unsubstituted heteroarylalkyl group having 3 to 30 carbon atoms, a substituted or unsubstituted alkyl group having 1 to 30 carbon atoms, and a substituted or unsubstituted cycloalkyl group having 3 to 20 carbon atoms.

4. The organic compound of claim 1, wherein Ar₂ is one selected from the group consisting of a substituted or unsubstituted phenyl group, a substituted or unsubstituted biphenyl group, a substituted or unsubstituted terphenyl group, a substituted or unsubstituted naphthyl group, a substituted or unsubstituted phenanthrenyl group, a substituted or unsubstituted fluorenyl group, a substituted or unsubstituted spirofluorenyl group, a substituted or unsubstituted dibenzofuranyl group, a substituted or unsubstituted dibenzothiophenyl group, a substituted or unsubstituted carbazolyl group, a substituted or unsubstituted cyclohexyl group, a substituted or unsubstituted napthobenzofuranyl group, and a substituted or unsubstituted napthobenzothiophenyl group.

5. The organic compound of claim 1, wherein L₁ to L₃ are each independently one of a single bond or a substituted or unsubstituted phenylene group.

6. The organic compound of claim 1, wherein a substituted or unsubstituted carbon ring structure formed by bonding two adjacent groups among R₁ to R₂₀ has a monocyclic or polycyclic aryl group structure.

7. An organic light emitting diode comprising:
a first electrode;
a second electrode facing the first electrode; and
an organic layer disposed between the first electrode and the second electrode, wherein the organic layer includes one or more of a hole transport layer and a hole transport auxiliary layer, and
wherein the one or more of the hole transport layer and the hole transport auxiliary layer include the organic compound of claim 1.

8. The organic light emitting diode of claim 7, wherein the hole transport auxiliary layer includes the organic compound of claim 1.

9. The organic light emitting diode of claim 7, wherein the hole transport layer includes the organic compound of claim 1.

10. The organic light emitting diode of claim 7, wherein the organic layer further includes one or more among a hole injection layer, a light emitting layer, an electron transport layer, and an electron injection layer.

11. The organic light emitting diode of claim 7, wherein a capping layer is formed on a surface of a cathode.

12. The organic light emitting diode of claim 11, wherein a protecting film is formed on the capping layer.

13. The organic light emitting diode of claim 7, wherein the light emitting layer is any one of a blue light emitting layer, a green light emitting layer, and a red light emitting layer.

14. The organic light emitting diode of claim 7, wherein the organic light emitting diode is used in any one selected from a flat panel display device, a flexible display device, a monochromatic or white flat panel lighting device, a monochromatic or white flexible lighting device, a vehicle display device, and a display device for virtual or augmented reality.
